(19) United States
(12) Patent Application Publication
(10) Pub. No.: US 2025/0313631 A1
(43) Pub. Date: Oct. 9, 2025

(54) TREATMENT AND PREVENTION OF CANCER USING VISTA ANTIGEN-BINDING MOLECULES

(71) Applicant: Hummingbird Bioscience Pte. Ltd., Singapore (SG)

(72) Inventors: Jerome Boyd-Kirkup, Singapore (SG); Siyu Guan, Singapore (SG); Konrad Paszkiewicz, Singapore (SG); Eric Rowinsky, Houston, TX (US); Dipti Thakkar, Singapore (SG); Bhushan Dharmadhikari, Singapore (SG); Piers Ingram, Singapore (SG)

(73) Assignee: Hummingbird Bioscience Pte. Ltd., Singapore (SG)

(21) Appl. No.: 18/692,314

(22) PCT Filed: Sep. 16, 2022
(86) PCT No.: PCT/EP2022/075849

§ 371 Date: Mar. 14, 2024

Related U.S. Application Data
(60) Provisional application No. 63/244,986, filed on Sep. 16, 2021.

(51) Int. Cl. C07K 16/28; A61K 9/08; A61K 39/00; A61K 47/12; A61K 47/22; A61K 47/26; A61P 35/00

(52) U.S. Cl. CPC C07K 16/2827; A61K 9/08; A61K 47/12; A61K 47/22; A61K 47/26; A61P 35/00; A61K 2039/505; A61K 2039/545

(57) ABSTRACT

The present disclosure provides antigen-binding molecules that bind to VISTA for the treatment or prevention of cancers, compositions comprising said molecules, and therapeutic and prophylactic methods using said molecules.

### RELATED APPLICATIONS

This application is a national stage filing under 35 U.S.C. § 371 of international PCT application PCT/EP2022/075849, filed Sep. 16, 2022, which claims priority from U.S. 63/244,986 filed Sep. 16, 2021, the entire contents and elements of each of which are herein incorporated by reference for all purposes.

### REFERENCE TO AN ELECTRONIC SEQUENCE LISTING

The contents of the electronic sequence listing (H096970008US01-SEQ-AZW.xml; Size: 317,513 bytes; and Date of Creation: Mar. 4, 2024) is herein incorporated by reference in its entirety.

This application claims priority from U.S. 63/244,986 filed 16 Sep. 2021, the contents and elements of which are herein incorporated by reference for all purposes.

### FIELD OF THE INVENTION

The present invention relates to the fields of molecular biology, more specifically antibody technology. The present invention also relates to methods of medical treatment and prophylaxis.

### BACKGROUND TO THE INVENTION

Myeloid Derived Suppressor Cell (MDSC)-mediated suppression of immune response has been identified in multiple solid tumors and lymphomas. MDSCs are elevated in advanced colorectal cancer (Toor et al, Front Immunol. 2016; 7:560). MDSCs are also observed in breast cancer, and the percentage of MDSCs in the peripheral blood is increased in patients with later stage breast cancer (Markowitz et al, Breast Cancer Res Treat. 2013 July; 140(1): 13-21). MDSC abundance is also correlated with poor prognosis in solid tumors (Charoentong et al, Cell Rep. 2017 Jan. 3; 18(1): 248-262).

MDSCs exert suppression over T cells through multiple mechanisms, including the production of reactive oxygen species, nitric oxide, and arginase. These ultimately lead to suppression of DC, NK and T cell activity and increased tumor burden (Umansky et al., Vaccines (Basel) (2016) 4(4): 36). MDSCs also contribute to the tumor development and metastasis through the production of soluble factors such as matrix metalloproteinases, VEGF, bFGF, TGF-β and S100A8/A9 which promote neovascularisation, invasion, proliferation and metastasis.

Targeting V-type immunoglobulin domain-containing suppressor of T-cell activation (VISTA), an immune checkpoint molecule expressed primarily on MDSCs, is an attractive therapeutic strategy for removing MDSC-mediated suppression of effector immune cell function.

WO 2017/137830 A1 discloses anti-VISTA antibody VSTB174, which is disclosed at e.g. paragraph to comprise the variable regions of anti-VISTA antibody VSTB112. Paragraph discloses that VSTB123 comprises the variable regions of VSTB174. Example 25 of WO 2017/137830 A1 at paragraph and FIG. 42A disclose that mIgG2a antibody VSTB123 was able to inhibit tumor growth in a MB49 tumor model. Paragraph and FIG. 42A disclose that by contrast VSTB124-which is the same antibody provided in IgG2a LALA format; see paragraph-did not inhibit tumor growth. Based on these results Example 25 concludes at paragraph that efficacy with anti-VISTA antibody treatment might require active Fc. Accordingly, the proposed mechanism of action for the anti-VISTA antibody represented schematically at FIG. 47 (see the legend to FIG. 47 at paragraph [0053]) involves Fc-mediated engagement of FcγRIII expressed by NK cells.

Hamster monoclonal anti-VISTA antibody mAb13F3 is disclosed in Le Mercier et al. Cancer Res. (2014) 74(7): 1933-44 to inhibit tumor growth in B16OVA and B16-BL6 melanoma models. Page 1942, paragraph spanning left and right columns teaches that immunogenicity and the FcR binding activity of the VISTA mAb might be critical limiting factors for achieving optimal target neutralization and therapeutic efficacy.

### SUMMARY OF THE INVENTION

In a first aspect the present invention provides an antigen-binding molecule, optionally isolated, which is capable of binding to VISTA and inhibiting VISTA-mediated signalling, independently of Fc-mediated function.

In some embodiments, the antigen-binding molecule comprises:
(i) a heavy chain variable (VH) region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278; and
(ii) a light chain variable (VL) region incorporating the following CDRs:
   LC-CDR1 having the amino acid sequence of SEQ ID NO:41
   LC-CDR2 having the amino acid sequence of SEQ ID NO:309
   LC-CDR3 having the amino acid sequence of SEQ ID NO:43.

In some embodiments, the antigen-binding molecule comprises:
(i) a heavy chain variable (VH) region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278; and
(ii) a light chain variable (VL) region incorporating the following CDRs:
   LC-CDR1 having the amino acid sequence of SEQ ID NO:41
   LC-CDR2 having the amino acid sequence of SEQ ID NO:295
   LC-CDR3 having the amino acid sequence of SEQ ID NO:43.

In some embodiments, the antigen-binding molecule comprises:
a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:310.

In some embodiments, the antigen-binding molecule comprises:
a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:297.

In some embodiments, the antigen-binding molecule comprises:
a VH region incorporating the following framework regions (FRs):
HC-FR1 having the amino acid sequence of SEQ ID NO:63
HC-FR2 having the amino acid sequence of SEQ ID NO:292
HC-FR3 having the amino acid sequence of SEQ ID NO:293
HC-FR4 having the amino acid sequence of SEQ ID NO:281.

In some embodiments, the antigen-binding molecule comprises:
a VL region incorporating the following framework regions (FRs):
LC-FR1 having the amino acid sequence of SEQ ID NO:288
LC-FR2 having the amino acid sequence of SEQ ID NO:298
LC-FR3 having the amino acid sequence of SEQ ID NO:284
LC-FR4 having the amino acid sequence of SEQ ID NO:47.

In some embodiments, the antigen-binding molecule comprises a heavy chain comprising the amino acid sequence of SEQ ID NO:331. In some embodiments, the antigen-binding molecule comprises a light chain comprising the amino acid sequence of SEQ ID NO:317.

In another aspect the present invention provides a composition comprising an antigen-binding molecule according to the present disclosure.

In some embodiments, the composition comprises:
(i) 2 mM to 200 mM histidine, 2% to 20% (w/v) sucrose, 0.001% to 0.1% (w/v) polysorbate-80, and has a pH 4.0 to 7.0; or
(ii) 2 mM to 200 mM histidine, 2% to 20% (w/v) sucrose, 0.001% to 0.1% (w/v) polysorbate-20, and has a pH 4.0 to 7.0; or
(iii) 2 mM to 200 mM histidine, 1 mM to 250 mM sodium chloride, and has a pH 4.0 to 7.0, optionally comprising 0.001% to 0.1% (w/v) polysorbate-20 or polysorbate-80; or
(iv) 2 mM to 200 mM histidine, 0.001% to 0.1% (w/v) polysorbate-20 or polysorbate-80, and has a pH 4.0 to 7.0; or
(v) 2 mM to 200 mM acetate, 2% to 20% (w/v) sucrose, 0.001% to 0.1% (w/v) polysorbate-80, and has a pH 4.0 to 7.0; or
(vi) 2 mM to 200 mM acetate, 2% to 20% (w/v) sucrose, 0.001% to 0.1% (w/v) polysorbate-20, and has a pH 4.0 to 7.0; or
(vii) 2 mM to 200 mM succinate, 2% to 20% (w/v) sucrose, 0.001% to 0.1% (w/v) polysorbate-80, and has a pH 4.0 to 7.0; or
(viii) 2 mM to 200 mM succinate, 2% to 20% (w/v) sucrose, 0.001% to 0.1% (w/v) polysorbate-20, and has a pH 4.0 to 7.0.

In some embodiments, the composition comprises:
(i) 20 mM histidine, 8% (w/v) sucrose; 0.02% (w/v) polysorbate-80, and has a pH 5.5; or
(ii) 20 mM histidine, 8% (w/v) sucrose; 0.02% (w/v) polysorbate-80, and has a pH 5.8; or
(iii) 20 mM histidine, 4% (w/v) sucrose; 0.02% (w/v) polysorbate-80, and has a pH 5.8; or
(iv) 20 mM histidine, 2% (w/v) sucrose; 0.02% (w/v) polysorbate-80, and has a pH 5.8; or
(v) 20 mM histidine, 8% (w/v) sucrose; 0.02% (w/v) polysorbate-80, and has a pH 6.3; or
(vi) 20 mM histidine, 8% (w/v) sucrose; 0.02% (w/v) polysorbate-20, and has a pH 5.8; or
(vii) 20 mM acetate, 8% (w/v) sucrose; 0.02% (w/v) polysorbate-80, and has a pH 5.5; or
(viii) 20 mM succinate, 8% (w/v) sucrose; 0.02% (w/v) polysorbate-80, and has a pH 5.5; or
(ix) 20 mM histidine, 0.02% (w/v) polysorbate-80, and has a pH 5.8; or
(x) 20 mM histidine, 150 mM sodium chloride, and has a pH 5.8.

In some embodiments, the composition comprises 20 mM histidine, 8% (w/v) sucrose; 0.02% (w/v) polysorbate-80, and has a pH 5.5.

In some embodiments, the composition comprises about 50 mg/ml (e.g. 50 mg/m) of the antigen-binding molecule.

In some aspects, an antigen-binding molecule or composition according to the present disclosure is provided for use as a medicament.

In some aspects, an antigen-binding molecule or composition according to the present disclosure is provided for use in a method of treating or preventing a cancer in a subject.

In some aspects, provided is the use of an antigen-binding molecule or composition according to the present disclosure in the manufacture of a medicament for treating or preventing a cancer in a subject.

In some aspects, provided is a method of treating or preventing a cancer in a subject, the method comprising administering a therapeutically- or prophylactically-effective amount of the antigen-binding molecule or composition according to the present disclosure.

In some embodiments, the cancer is characterised by the presence of cells expressing VISTA and/or by signalling mediated by a complex comprising VISTA.

In some embodiments, the cancer is selected from: a hematological cancer, leukemia (e.g. T cell leukemia), acute myeloid leukemia, lymphoma, B cell lymphoma, T cell lymphoma, multiple myeloma, mesothelioma, a solid tumour, lung cancer, non-small cell lung carcinoma (NSCLC), gastric cancer, gastric carcinoma, colorectal cancer, colorectal carcinoma, colorectal adenocarcinoma, uterine cancer, uterine corpus endometrial carcinoma, breast cancer, triple negative breast cancer (TBNC), triple negative breast invasive carcinoma, invasive ductal carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, bladder cancer, prostate cancer and/or prostate adenocarcinoma. In some embodiments, the cancer is triple negative breast cancer (TBNC), non-small cell lung carcinoma (NSCLC) and/or a solid tumour.

In some embodiments, the treatment or prevention of cancer additionally comprises administering an agent capable of inhibiting signalling mediated by an immune checkpoint molecule other than VISTA, e.g. wherein the immune checkpoint molecule other than VISTA is PD-1 and/or PD-L1. The agent may be an anti-PD-1 or anti-PD-L1 antibody.

In some embodiments, the treatment or prevention, or method thereof, comprises a step of detecting the presence of cells expressing VISTA and/or by signalling mediated by a complex comprising VISTA. In some embodiments, the subject is selected for treatment with the antigen-binding molecule or composition when the presence of cells expressing VISTA and/or signalling mediated by a complex comprising VISTA is detected.

In some embodiments, the antigen-binding molecule is administered weekly, e.g. in a composition according to the present disclosure. In some embodiments, the antigen-binding molecule is administered one, two or three times within an administration cycle of 21 days, optionally wherein the treatment comprises up to 35 administration cycles. In some embodiments, the antigen-binding molecule is administered on days 1, 8 and/or 15 within an administration cycle of 21 days, optionally wherein the treatment comprises up to 35 administration cycles. In some embodiments, the antigen-binding molecule is administered on days 1, 8, 15 and/or 22 within an administration cycle of 28 days, optionally wherein the treatment comprises up to 35 administration cycles.

In some embodiments, the treatment or prevention, or method thereof, comprises administering 3.5 mg to 2200 mg of antigen-binding molecule per administration.

In some embodiments, the treatment or prevention, or method thereof, comprises administering (up to or at least) 3.5 mg, 7 mg, 10.5 mg, 17.5 mg, 20 mg, 21 mg, 40 mg, 60 mg, 72 mg, 120 mg, 180 mg, 240 mg, 360 mg, 400 mg, 800 mg, 1200 mg, 1600 mg, 1900 mg or 2200 mg of antigen-binding molecule (e.g. in a composition according to the present disclosure) per administration, e.g. according to an administration schedule of the present disclosure.

In some embodiments, the treatment or prevention, or method thereof, comprises administering up to 10.5 mg, up to 21 mg, up to 31.5 mg, up to 52.5 mg, up to 60 mg, up to 63 mg, up to 120 mg, up to 180 mg, up to 216 mg, up to 360 mg, up to 540 mg, up to 720 mg, up to 1080 mg, up to 1200 mg, up to 2400 mg, up to 3600 mg, up to 4800 mg, up to 5700 mg, or up to 6600 mg of antigen-binding molecule (e.g. in a composition according to the present disclosure) per administration cycle of 21 days.

### DESCRIPTION

The present invention relates to novel VISTA-binding molecules having novel and/or improved properties as compared to known anti-VISTA antibodies.

The inventors generated antigen-binding molecules which bind to particular regions of interest in the extracellular region of VISTA. The VISTA-binding molecules of the present invention are provided with combinations of desirable biophysical and functional properties as compared to VISTA-binding antigen-binding molecules disclosed in the prior art.

In particular, VISTA-binding molecules described herein are demonstrated to be capable of antagonising VISTA-mediated signalling through a mechanism that does not require Fc-mediated functions. The inventors demonstrate that VISTA-binding molecules described herein comprising Fc which lack the ability to bind to Fcγ receptors and/or C1q are able to provide therapeutic anti-cancer effects in vivo.

The inventors establish for the first time that it is possible to antagonise VISTA-mediated signalling directly through a mechanism that does not require Fc-mediated effector function (e.g. ADCC/ADCP/CDC directed against VISTA-expressing cells).

The VISTA-binding molecules of the present disclosure target a region of VISTA that is different from the region targeted by known anti-VISTA antibodies. Antigen-binding molecules targeting the particular region of VISTA are able to antagonise VISTA-mediated signalling without the requirement for Fc-mediated effector functions.

VISTA-binding molecules disclosed herein are therefore useful for inhibiting VISTA-mediated signalling without depleting VISTA expressing cells. This is important, because VISTA is expressed on cells which it is not desirable to deplete. VISTA-binding molecules disclosed herein are thus able to inhibit VISTA-mediated signalling whilst minimising undesirable side effects.

VISTA-binding molecules disclosed herein are also advantageously shown to be capable of releasing T cells from VISTA-mediated suppression. Specifically, the VISTA-binding molecules disclosed herein are shown to be able to increase T cell proliferation, and production of e.g. IFNγ and TNFa from T cells cultured in the presence of VISTA or VISTA-expressing cells.

### VISTA, Binding Partners and VISTA-Mediated Signalling

V-type immunoglobulin domain-containing suppressor of T-cell activation (VISTA; also known e.g. as B7-H5, SISP1, PD-1H) is the protein identified by UniProt Q9H7M9, having the amino acid sequence shown in SEQ ID NO:1 (Q9H7M9-1, v3). The structure and function of VISTA is described e.g. in Lines et al., Cancer Res. (2014) 74(7): 1924-1932, which is hereby incorporated by reference in its entirety. VISTA is a ˜50 kDa single-pass type I transmembrane that functions as an immune checkpoint and is encoded by the C10orf54 gene. The extracellular domain of VISTA is homologous to PD-L1.

The N-terminal 32 amino acids of SEQ ID NO:1 constitutes a signal peptide, and so the mature form of VISTA (i.e. after processing to remove the signal peptide) has the amino acid sequence shown in SEQ ID NO: 2. Positions 33 to 194 of SEQ ID NO:1 form the extracellular domain (SEQ ID NO:3), positions 195 to 215 form a transmembrane domain (SEQ ID NO:4), and positions 216 to 311 form the cytoplasmic domain (SEQ ID NO:5). The extracellular domain comprises an Ig-like V-type domain (positions 33 to 168 of SEQ ID NO:1, shown in SEQ ID NO:6).

In this specification “VISTA” refers to VISTA from any species and includes VISTA isoforms, fragments, variants (including mutants) or homologues from any species.

As used herein, a “fragment”, “variant” or “homologue” of a protein may optionally be characterised as having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference protein (e.g. a reference isoform). In some embodiments fragments, variants, isoforms and homologues of a reference protein may be characterised by ability to perform a function performed by the reference protein.

A “fragment” generally refers to a fraction of the reference protein. A “variant” generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An “isoform” generally refers to a variant of the reference protein expressed by the same species as the species of the reference protein. A “homologue” generally refers to a variant of the reference protein produced by a different species as compared to the species of the reference protein. Homologues include orthologues.

A “fragment” may be of any length (by number of amino acids), although may optionally be at least 20% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein. A fragment of VISTA may have a minimum length of one of 10, 20, 30, 40, 50, 100, 150, 200, 250 or 300 amino acids, and may have a maximum length of one of 20, 30, 40, 50, 100, 150, 200, 250 or 300 amino acids.

In some embodiments, the VISTA is VISTA from a mammal (e.g. a primate (rhesus, cynomolgous, non-human primate or human) and/or a rodent (e.g. rat or murine) VISTA). Isoforms, fragments, variants or homologues of VISTA may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature VISTA isoform from a given species, e.g. human.

Isoforms, fragments, variants or homologues may optionally be functional isoforms, fragments, variants or homologues, e.g. having a functional property/activity of the reference VISTA, as determined by analysis by a suitable assay for the functional property/activity. For example, an isoform, fragment, variant or homologue of VISTA may e.g. display association with VSIG-3, LRIG1, VSIG8 and/or PSGL-1.

In some embodiments, the VISTA comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:1 or 2. In some embodiments, a fragment of VISTA comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs: 2, 3 or 6.

VISTA is a member of the B7 family of proteins, and is primarily expressed by leukocytes, and in particular CD14+ monocytes (including monocyte-derived suppressor cells (MDSCs)) and CD33+ myeloid cells. VISTA is also expressed by CD56+NK cells, dendritic cells, and to a lesser extent on CD4+ and CD8+ T cells. VISTA is highly expressed on MDSCs, in particular tumor-infiltrating MDSCs, and also on tumor-infiltrating myeloid DCs (Le Mercier et al, Cancer Res. (2014) 74(7): 1933-44), as well as on tumor-associated macrophages (TAMs) and neutrophils.

There is evidence that VISTA can act as both a ligand and a receptor on T cells to inhibit T cell effector function and maintain peripheral tolerance; tumors engineered to overexpress VISTA evade immune control and grow faster than tumors which do not overexpress VISTA (Wang et al., Journal of Experimental Medicine. (2011) 208(3): 577-92; Lines et al., Cancer Res. (2014) 74(7): 1924-1932). VISTA has been shown to be a co-inhibitory receptor on CD4+ T cells or a co-inhibitory ligand for T cells. VISTA^{-/-} CD4+ T cells have been reported to display stronger antigen-specific proliferation and cytokine production than wildtype CD4+ T cells, suggesting that VISTA functions as an inhibitory receptor on CD4+ T cells. Blocking VISTA function using monoclonal anti-VISTA antibody has been shown to enhance infiltration, proliferation and effector function of tumor-reactive T cells within the tumor microenvironment (Le Mercier et al, Cancer Res. (2014) 74(7): 1933-4).

VISTA has been proposed to interact with VSIG-3 (IGSF11)—see e.g. Wang et al., J Immunol (2017), 198 (1 Supplement) 154.1, which is hereby incorporated by reference in its entirety. Engagement of VSIG-3 through VISTA on activated T cells inhibits T cell proliferation, and reduces production of cytokines and chemokines such as IFN-γ, IL-2, IL-17, CCL5/RANTES, CCL3/MIP-1a, and CXCL11/I-TAC.

VSIG-3 is the protein identified by UniProt Q5DX21. Alternative splicing of mRNA encoded by the human IGSF11 gene yields three different isoforms: isoform 1 (UniProt: Q5DX21-1, v3; SEQ ID NO:7); isoform 2 (UniProt: Q5DX21-2; SEQ ID NO:8), which comprises a different sequence to SEQ ID NO:7 at positions 1 to 17; and isoform 3 (UniProt: Q5DX21-3; SEQ ID NO:9), which comprises a different sequence to SEQ ID NO: 7 at positions 1 to 17, and which also comprises a different sequence to SEQ ID NO:7 at positions 211-235.

The N-terminal 22 amino acids of SEQ ID NOs: 7, 8 and 9 constitute a signal peptide, and so the mature form of VSIG-3 isoforms 1, 2 and 3 (i.e. after processing to remove the signal peptide) have the amino acid sequences shown in SEQ ID NOs: 10, 11 and 12, respectively. Positions 23 to 241 of SEQ ID NOs: 7, and 8 form the extracellular domain of VSIG-3 isoforms 1 and 2 (SEQ ID NO:13), and positions 23 to 216 of SEQ ID NO:9 form the extracellular domain of VSIG-3 isoform 3 (SEQ ID NO:14). The transmembrane domain of VSIG-3 is shown in SEQ ID NO:15, and the cytoplasmic domain is shown in SEQ ID NO:16. The extracellular domain comprises an Ig-like V-type domain (shown in SEQ ID NO:17), and the extracellular domains of VSIG-3 isoforms 1 and 2 additionally comprise an Ig-like C2-type domain (shown in SEQ ID NO:18).

In this specification “VSIG-3” refers to VSIG-3 from any species and includes VSIG-3 isoforms, fragments, variants (including mutants) or homologues from any species.

A fragment of VSIG-3 may have a minimum length of one of 10, 20, 30, 40, 50, 100, 150, 200, 250, 300, 350 or 400 amino acids, and may have a maximum length of one of 20, 30, 40, 50, 100, 150, 200, 250, 300, 350 or 400 amino acids.

In some embodiments, the VSIG-3 is VSIG-3 from a mammal (e.g. a primate (rhesus, cynomolgous, non-human primate or human) and/or a rodent (e.g. rat or murine) VSIG-3). Isoforms, fragments, variants or homologues of VSIG-3 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature VSIG-3 isoform from a given species, e.g. human.

Isoforms, fragments, variants or homologues may optionally be functional isoforms, fragments, variants or homologues, e.g. having a functional property/activity of the reference VSIG-3, as determined by analysis by a suitable assay for the functional property/activity. For example, an isoform, fragment, variant or homologue of VSIG-3 may e.g. display association with VISTA.

In some embodiments, the VSIG-3 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs: 7 to 12. In some embodiments, a fragment of VSIG-3 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs: 10 to 14, 17 or 18.

VISTA has also been proposed to interact with VSIG-8-see e.g. WO/2016/090347 A1. VSIG-8 is the protein identified by UniProt P0DPA2 (SEQ ID NO:19). The N-terminal 21 amino acids of SEQ ID NO:19 constitutes a signal peptide, and so the mature form of VSIG-8 (i.e. after processing to remove the signal peptide) has the amino acid sequence shown in SEQ ID NO:20. Positions 22 to 263 of SEQ ID NO:19 form the extracellular domain of VSIG-8 (SEQ ID NO:21). The transmembrane domain of VSIG-8 is shown in SEQ ID NO:22, and the cytoplasmic domain is shown in SEQ ID NO:23. The extracellular domain comprises an Ig-like V-type domain 1 (shown in SEQ ID NO:24), and an Ig-like V-type domain 2 (shown in SEQ ID NO:25).

In this specification “VSIG-8” refers to VSIG-8 from any species and includes VSIG-8 isoforms, fragments, variants (including mutants) or homologues from any species.

A fragment of VSIG-8 may have a minimum length of one of 10, 20, 30, 40, 50, 100, 150, 200, 250, 300, 350 or 400 amino acids, and may have a maximum length of one of 20, 30, 40, 50, 100, 150, 200, 250, 300, 350 or 400 amino acids.

In some embodiments, the VSIG-8 is VSIG-8 from a mammal (e.g. a primate (rhesus, cynomolgous, non-human primate or human) and/or a rodent (e.g. rat or murine) VSIG-8). Isoforms, fragments, variants or homologues of VSIG-8 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature VSIG-8 isoform from a given species, e.g. human.

Isoforms, fragments, variants or homologues may optionally be functional isoforms, fragments, variants or homologues, e.g. having a functional property/activity of the reference VSIG-8, as determined by analysis by a suitable assay for the functional property/activity. For example, an isoform, fragment, variant or homologue of VSIG-8 may e.g. display association with VISTA.

In some embodiments, the VSIG-8 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:19 or 20. In some embodiments, a fragment of VSIG-8 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs: 20, 21, 24 or 25.

VISTA has also been proposed to interact with PSGL-1-see e.g. WO 2018/132476 A1. PSGL-1 isoform 1 is the protein identified by UniProt Q14242-1 (SEQ ID NO:323). PSGL-1 isoform 2 is the protein identified by UniProt Q14242-2 (SEQ ID NO:324), and differs from PSGL-1 isoform 1 in that it comprises an additional 16 amino acids after position 1 of SEQ ID NO:323.

The N-terminal 17 amino acids of SEQ ID NO:323 constitutes a signal peptide, and so the mature form of PSGL-1 (i.e. after processing to remove the signal peptide) has the amino acid sequence shown in SEQ ID NO: 325. Positions 18 to 320 of SEQ ID NO:323 form the extracellular domain of PSGL-1 (SEQ ID NO: 326). The transmembrane domain of PSGL-1 is shown in SEQ ID NO:327, and the cytoplasmic domain is shown in SEQ ID NO:328. The extracellular domain comprises 12, 10 amino acid tandem repeats; the repeat region is shown in SEQ ID NO:329.

In this specification “PSGL-1” refers to PSGL-1 from any species and includes PSGL-1 isoforms, fragments, variants (including mutants) or homologues from any species.

A fragment of PSGL-1 may have a minimum length of one of 10, 20, 30, 40, 50, 100, 150, 200, 250, 300, 350 or 400 amino acids, and may have a maximum length of one of 20, 30, 40, 50, 100, 150, 200, 250, 300, 350 or 400 amino acids.

In some embodiments, the PSGL-1 is PSGL-1 from a mammal (e.g. a primate (rhesus, cynomolgous, non-human primate or human) and/or a rodent (e.g. rat or murine) PSGL-1). Isoforms, fragments, variants or homologues of PSGL-1 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature PSGL-1 isoform from a given species, e.g. human.

Isoforms, fragments, variants or homologues may optionally be functional isoforms, fragments, variants or homologues, e.g. having a functional property/activity of the reference PSGL-1, as determined by analysis by a suitable assay for the functional property/activity. For example, an isoform, fragment, variant or homologue of PSGL-1 may e.g. display association with VISTA.

In some embodiments, the PSGL-1 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:323 or 324. In some embodiments, a fragment of PSGL-1 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs: 325, 326 or 329.

### Regions of Particular Interest on the Target Molecule

The antigen-binding molecules of the present invention were specifically designed to target regions of VISTA of particular interest. In a two-step approach, VISTA regions to be targeted were selected following analysis for predicted antigenicity, function and safety. Antibodies specific for the target regions of VISTA were then prepared using peptides corresponding to the target regions as immunogens to raise specific monoclonal antibodies, and subsequent screening to identify antibodies capable of binding to VISTA in the native state. This approach provides exquisite control over the antibody epitope.

The antigen-binding molecules of the present invention may be defined by reference to the region of VISTA which they bind to. The antigen-binding molecules of the present invention may bind to a particular region of interest of VISTA. In some embodiments the antigen-binding molecule may bind to a linear epitope of VISTA, consisting of a contiguous sequence of amino acids (i.e. an amino acid primary sequence). In some embodiments, the antigen-binding molecule may bind to a conformational epitope of VISTA, consisting of a discontinuous sequence of amino acids of the amino acid sequence.

In some embodiments, the antigen-binding molecule of the present invention binds to VISTA. In some embodiments, the antigen-binding molecule binds to the extracellular region of VISTA (e.g. the region shown in SEQ ID NO:3). In some embodiments, the antigen-binding molecule binds to the Ig-like V-type domain of VISTA (e.g. the region shown in SEQ ID NO:6). In some embodiments, the antigen-binding molecule binds to VISTA in the region corresponding to positions 61 to 162 of SEQ ID NO:1 (shown in SEQ ID NO:31).

In some embodiments, the antigen-binding molecule binds to the region of VISTA shown in SEQ ID NO: 322. In some embodiments, the antigen-binding molecule binds to the region of VISTA shown in SEQ ID NO: 26. In some embodiments, the antigen-binding molecule binds to the region of VISTA shown in SEQ ID NO:27. In some embodiments, the antigen-binding molecule binds to the region of VISTA shown in SEQ ID NO:28. In some embodiments, the antigen-binding molecule binds to the region of VISTA shown in SEQ ID NO:29. In some embodiments, the antigen-binding molecule binds to the region of VISTA shown in SEQ ID NO:30.

In some embodiments, the antigen-binding molecule does not bind to the region of VISTA shown in SEQ ID NO: 271. In some embodiments, the antigen-binding molecule does not bind to the region of VISTA shown in SEQ ID NO:272. In some embodiments, the antigen-binding molecule does not bind to the region of VISTA shown in SEQ ID NO:273. In some embodiments, the antigen-binding molecule does not bind to the region of VISTA shown in SEQ ID NO:274. In some embodiments, the antigen-binding molecule does not bind to the region of VISTA shown in SEQ ID NO:275.

The region of a peptide/polypeptide to which an antibody binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3): 145-156, which is hereby incorporated by reference in its entirety.

In some embodiments the antigen-binding molecule is capable of binding the same region of VISTA, or an overlapping region of VISTA, to the region of VISTA which is bound by an antibody comprising the VH and VL sequences of one of antibody clones 4M2-C12, 4M2-B4, 4M2-C9, 4M2-D9, 4M2-D5, 4M2-A8, V4H1, V4H2, V4-C1, V4-C9, V4-C24, V4-C26, V4-C27, V4-C28, V4-C30, V4-C31, 2M1-B12, 2M1-D2, 1M2-D2, 13D5p, 13D5-1, 13D5-13, 5M1-A11 or 9M2-C12 described herein.

As used herein, a “peptide” refers to a chain of two or more amino acid monomers linked by peptide bonds. A peptide typically has a length in the region of about 2 to 50 amino acids. A “polypeptide” is a polymer chain of two or more peptides. Polypeptides typically have a length greater than about 50 amino acids.

In some embodiments, the antigen-binding molecule of the present invention is capable of binding to a polypeptide comprising, or consisting of, the amino acid sequence of one of SEQ ID NOs: 1, 2, 3, 6 or 31.

In some embodiments, the antigen-binding molecule is capable of binding to a peptide/polypeptide comprising, or consisting of, the amino acid sequence of SEQ ID NO:322. In some embodiments, the antigen-binding molecule is capable of binding to a peptide/polypeptide comprising, or consisting of, the amino acid sequence of SEQ ID NO:26. In some embodiments, the antigen-binding molecule is capable of binding to a peptide/polypeptide comprising, or consisting of, the amino acid sequence of SEQ ID NO: 27. In some embodiments, the antigen-binding molecule is capable of binding to a peptide/polypeptide comprising, or consisting of, the amino acid sequence of SEQ ID NO:28. In some embodiments, the antigen-binding molecule is capable of binding to a peptide/polypeptide comprising, or consisting of, the amino acid sequence of SEQ ID NO:29. In some embodiments, the antigen-binding molecule is capable of binding to a peptide/polypeptide comprising, or consisting of, the amino acid sequence of SEQ ID NO:30.

In some embodiments, the antigen-binding molecule is not capable of binding to a peptide consisting of the amino acid sequence of SEQ ID NO:271. In some embodiments, the antigen-binding molecule is not capable of binding to a peptide consisting of the amino acid sequence of SEQ ID NO:272. In some embodiments, the antigen-binding molecule is not capable of binding to a peptide consisting of the amino acid sequence of SEQ ID NO:273. In some embodiments, the antigen-binding molecule is not capable of binding to a peptide consisting of the amino acid sequence of SEQ ID NO:274. In some embodiments, the antigen-binding molecule is not capable of binding to a peptide consisting of the amino acid sequence of SEQ ID NO:275.

The ability of an antigen-binding molecule to bind to a given peptide/polypeptide can be analysed by methods well known to the skilled person, including analysis by ELISA, immunoblot (e.g. western blot), immunoprecipitation, Surface Plasmon Resonance (SPR; see e.g. Hearty et al., Methods Mol Biol (2012) 907:411-442) or Bio-Layer Interferometry (see e.g. Lad et al., (2015) J Biomol Screen 20(4): 498-507).

In embodiments where the antigen binding molecule is capable of binding to a peptide/polypeptide comprising a reference amino acid sequence, the peptide/polypeptide may comprise one or more additional amino acids at one or both ends of the reference amino acid sequence. In some embodiments the peptide/polypeptide comprises e.g. 1-5, 1-10, 1-20, 1-30, 1-40, 1-50, 5-10, 5-20, 5-30, 5-40, 5-50, 10-20, 10-30, 10-40, 10-50, 20-30, 20-40 or 20-50 additional amino acids at one or both ends of the reference amino acid sequence.

In some embodiments the additional amino acid(s) provided at one or both ends (i.e. the N-terminal and C-terminal ends) of the reference sequence correspond to the positions at the ends of the reference sequence in the context of the amino acid sequence of VISTA. By way of example, where the antigen-binding molecule is capable of binding to a peptide/polypeptide comprising the sequence of SEQ ID NO: 26, and an additional two amino acids at the C-terminal end of SEQ ID NO:26, the additional two amino acids may be arginine and asparagine, corresponding to positions 90 and 91 of SEQ ID NO:1.

In some embodiments the antigen-binding molecule is capable of binding to a peptide/polypeptide which is bound by an antibody comprising the VH and VL sequences of one of antibody clones 4M2-C12, 4M2-B4, 4M2-C9, 4M2-D9, 4M2-D5, 4M2-A8, V4H1, V4H2, V4-C1, V4-C9, V4-C24, V4-C26, V4-C27, V4-C28, V4-C30, V4-C31, 2M1-B12, 2M1-D2, 1M2-D2, 13D5p, 13D5-1, 13D5-13, 5M1-A11 or 9M2-C12 described herein.

### Myeloid-Derived Suppressor Cells (MDSCs)

Myeloid-Derived Suppressor Cells (MDSCs) are a heterogeneous group of immune cells of the myeloid lineage of cells, characterised by an immunosuppressive phenotype. MDSC biology is reviewed in Kumar et al., Trends Immunol. (2016); 37(3): 208-220, which is hereby incorporated by reference in its entirety.

MDSC are characterised by a number of biochemical and genomic features that distinguish these cells from mature myeloid cells (i.e. macrophages, dendritic cells and neutrophils) such as: increased expression of NADPH oxidase (Nox2), increased production of reactive oxygen species (ROS) (such as superoxide anion (O²⁻), hydrogen peroxide (H₂O₂), and peroxynitrite (PNT; ONOO⁻)); increased expression of arginase 1 and nitric oxide synthase 2 (nos2), and increased production of nitric oxide (NO); increased expression of c/EBPB and STAT3; decreased expression of IRF8; and increased production of S100A8/9 proteins.

There are two different types of MDSC; polymorphonuclear MDSCs (PMN-MDSCs), which are morphologically and phenotypically similar to neutrophils, and monocytic MDSCs (M-MDSCs) which are more similar to monocytes. The morphologic and phenotypic characteristics of MDSCs are described e.g. in Marvel and Gabrilovich J Clin Invest. 2015 Sep. 1; 125(9): 3356-3364, which is hereby incorporated by reference in its entirety. In mice, MDSCs are broadly identified as CD11b⁺Gr1⁺ cells. Gr-1^{hi} cells are mostly PMN-MDSCs, and Gr-1^{lo} cells are mostly M-MDSCs. These subsets can be more accurately identified based on Ly6C and Ly6G markers; M-MDSCs are CD11b⁺Ly6C^{hi}Ly6G⁻, and PMN-MDSCs are CD11b⁺Ly6C^{lo}Ly6G⁺). In humans, MDSCs are identified in the mononuclear fraction. PMN-MDSCs are CD14⁻CD11b⁺CD33⁺CD15⁺ or CD66b⁺ cells, and M-MDSCs are CD14⁺HLA-DR⁻/^{lo} cells. Populations of Lin⁻HLA⁻DR⁻CD33⁺MDSCs represent a mixed group of cells enriched for myeloid progenitors.

Factors implicated in MDSC-mediated immune suppression include expression of arginase (ARG1), inducible NOS (iNOS), TGF-β, IL-10, and COX2, sequestration of cysteine, decreased expression of I-selectin by T cells, and induction of Tregs. M-MDSCs and PMN-MDSCs employ different mechanisms of immune suppression. M-MDSCs suppress both antigen-specific and non-specific T cell responses through production of NO and cytokines, and are more strongly immunosuppressive than PMN-MDSCs. PMN-MDSCs suppress immune responses in an antigen-specific manner through production of ROS. MDSCs are pathologically implicated in the development and progression of cancer and infectious disease. The role of MDSCs in human disease is reviewed e.g. in Kumar et al., Trends Immunol. (2016); 37(3): 208-220 (incorporated by reference herein) and Greten et al., Int Immunopharmacol. (2011) 11(7): 802-807, which is hereby incorporated by reference in its entirety.

MDSCs are abundant in tumor tissues, and contribute to the development and progression of cancer through multiple mechanisms, reviewed e.g. in Umansky et al., Vaccines (Basel) (2016) 4(4): 36. MDSCs are recruited to the tumor site through chemokine expression, and proinflammatory factors in the tumor microenvironment result in significant upregulation of immunosuppressive function by MDSCs. MDSCs contribute to tumor development, neovascularization and metastasis through suppression of effector immune cell function (e.g. effector T cell and NK cell function), promotion of regulatory T cell production/activity, production of growth factors such as VEGF and bFGF, and production of ECM-modifying factors such as matrix metalloproteinases.

MDSCs may be characterised by reference to expression of VISTA. In embodiments of the various aspects of the present invention, the MDSCs may be “VISTA-expressing MDSCs” or “VISTA+MDSCs”. The MDSCs may express VISTA at the cell surface (i.e. VISTA may be expressed in or at the cell membrane).

### Antigen-Binding Molecules

The present invention provides antigen-binding molecules capable of binding to VISTA.

An “antigen-binding molecule” refers to a molecule which is capable of binding to a target antigen, and encompasses monoclonal antibodies, polyclonal antibodies, monospecific and multispecific antibodies (e.g., bispecific antibodies), and antibody fragments (e.g. Fv, scFv, Fab, scFab, F(ab′)₂, Fab₂, diabodies, triabodies, scFv-Fc, minibodies, single domain antibodies (e.g. VhH), etc.), as long as they display binding to the relevant target molecule(s).

The antigen-binding molecule of the present invention comprises a moiety capable of binding to a target antigen(s). In some embodiments, the moiety capable of binding to a target antigen comprises an antibody heavy chain variable region (VH) and an antibody light chain variable region (VL) of an antibody capable of specific binding to the target antigen. In some embodiments, the moiety capable of binding to a target antigen comprises or consists of an aptamer capable of binding to the target antigen, e.g. a nucleic acid aptamer (reviewed, for example, in Zhou and Rossi Nat Rev Drug Discov. 2017 16(3): 181-202). In some embodiments, the moiety capable of binding to a target antigen comprises or consists of a antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)) affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

The antigen-binding molecules of the present invention generally comprise an antigen-binding domain comprising a VH and a VL of an antibody capable of specific binding to the target antigen. The antigen-binding domain formed by a VH and a VL may also be referred to herein as an Fv region.

An antigen-binding molecule may be, or may comprise, an antigen-binding polypeptide, or an antigen-binding polypeptide complex. An antigen-binding molecule may comprise more than one polypeptide which together form an antigen-binding domain. The polypeptides may associate covalently or non-covalently. In some embodiments the polypeptides form part of a larger polypeptide comprising the polypeptides (e.g. in the case of scFv comprising VH and VL, or in the case of scFab comprising VH-CH1 and VL-CL).

An antigen-binding molecule may refer to a non-covalent or covalent complex of more than one polypeptide (e.g. 2, 3, 4, 6, or 8 polypeptides), e.g. an IgG-like antigen-binding molecule comprising two heavy chain polypeptides and two light chain polypeptides.

The antigen-binding molecules of the present invention may be designed and prepared using the sequences of monoclonal antibodies (mAbs) capable of binding to VISTA. Antigen-binding regions of antibodies, such as single chain variable fragment (scFv), Fab and F(ab′)₂ fragments may also be used/provided. An “antigen-binding region” is any fragment of an antibody which is capable of binding to the target for which the given antibody is specific.

Antibodies generally comprise six complementarity-determining regions CDRs; three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody which binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibody clones described herein were defined according to the international IMGT (ImMunoGeneTics) information system (LeFranc et al., Nucleic Acids Res. (2015) 43 (Database issue): D413-22), which uses the IMGT V-DOMAIN numbering rules as described in Lefranc et al., Dev. Comp. Immunol. (2003) 27:55-77.

In some embodiments, the antigen-binding molecule comprises the CDRs of an antigen-binding molecule which is capable of binding to VISTA. In some embodiments, the antigen-binding molecule comprises the FRs of an antigen-binding molecule which is capable of binding to VISTA. In some embodiments, the antigen-binding molecule comprises the CDRs and the FRs of an antigen-binding molecule which is capable of binding to VISTA. That is, in some embodiments the antigen-binding molecule comprises the VH region and the VL region of an antigen-binding molecule which is capable of binding to VISTA.

In some embodiments the antigen-binding molecule comprises a VH region and a VL region which is, or which is derived from, the VH/VL region of a VISTA-binding antibody clone described herein (i.e. anti-VISTA antibody clones 4M2-C12, 4M2-B4, 4M2-C9, 4M2-D9, 4M2-D5, 4M2-A8, V4H1, V4H2, V4-C1, V4-C9, V4-C24, V4-C26, V4-C27, V4-C28, V4-C30, V4-C31, 2M1-B12, 2M1-D2, 1M2-D2, 13D5p, 13D5-1, 13D5-13, 5M1-A11 or 9M2-C12).

In some embodiments the antigen-binding molecule comprises a VH region according to one of (1) to (18) below:
(1) (4M2-C12 derived consensus) a VH region incorporating the following CDRs:
   HC-CDR1 having the amino acid sequence of SEQ ID NO:305
   HC-CDR2 having the amino acid sequence of SEQ ID NO:306
   HC-CDR3 having the amino acid sequence of SEQ ID NO:307,
or a variant thereof in which one or two or three amino acids in one or more of HC-CDR1, HC-CDR2, or HC-CDR3 are substituted with another amino acid.
(2) (V4-C24, V4-C26, V4-C27, V4-C28, V4-C30, V4-C31) a VH region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278,
or a variant thereof in which one or two or three amino acids in one or more of HC-CDR1, HC-CDR2, or HC-CDR3 are substituted with another amino acid.
(3) (V4-C1) a VH region incorporating the following CDRs:
   HC-CDR1 having the amino acid sequence of SEQ ID NO:33
   HC-CDR2 having the amino acid sequence of SEQ ID NO:277
   HC-CDR3 having the amino acid sequence of SEQ ID NO:278,
or a variant thereof in which one or two or three amino acids in one or more of HC-CDR1, HC-CDR2, or HC-CDR3 are substituted with another amino acid.
(4) (V4-C9) a VH region incorporating the following CDRs:
   HC-CDR1 having the amino acid sequence of SEQ ID NO:33
   HC-CDR2 having the amino acid sequence of SEQ ID NO:286
   HC-CDR3 having the amino acid sequence of SEQ ID NO:278,
or a variant thereof in which one or two or three amino acids in one or more of HC-CDR1, HC-CDR2, or HC-CDR3 are substituted with another amino acid.
(5) (4M2-C12/V4H1/V4H2 consensus) a VH region incorporating the following CDRs:
   HC-CDR1 having the amino acid sequence of SEQ ID NO:244
   HC-CDR2 having the amino acid sequence of SEQ ID NO:34
   HC-CDR3 having the amino acid sequence of SEQ ID NO:35,
   or a variant thereof in which one or two or three amino acids in one or more of HC-CDR1, HC-CDR2, or HC-CDR3 are substituted with another amino acid.
(6) (4M2-C12, 4M2-B4, V4H2) a VH region incorporating the following CDRs:
   HC-CDR1 having the amino acid sequence of SEQ ID NO:33
   HC-CDR2 having the amino acid sequence of SEQ ID NO:34
   HC-CDR3 having the amino acid sequence of SEQ ID NO:35,
   or a variant thereof in which one or two or three amino acids in one or more of HC-CDR1, HC-CDR2, or HC-CDR3 are substituted with another amino acid.
(7) (V4H1) a VH region incorporating the following CDRs:
   HC-CDR1 having the amino acid sequence of SEQ ID NO:53
   HC-CDR2 having the amino acid sequence of SEQ ID NO:34
   HC-CDR3 having the amino acid sequence of SEQ ID NO:35,
   or a variant thereof in which one or two or three amino acids in one or more of HC-CDR1, HC-CDR2, or HC-CDR3 are substituted with another amino acid.
(8) (2M1-B12, 2M1-D2) a VH region incorporating the following CDRs:
   HC-CDR1 having the amino acid sequence of SEQ ID NO:72
   HC-CDR2 having the amino acid sequence of SEQ ID NO:73
   HC-CDR3 having the amino acid sequence of SEQ ID NO:74,
   or a variant thereof in which one or two or three amino acids in one or more of HC-CDR1, HC-CDR2, or HC-CDR3 are substituted with another amino acid.
(9) (4M2-C9, 5M1-A11) a VH region incorporating the following CDRs:
   HC-CDR1 having the amino acid sequence of SEQ ID NO:88
   HC-CDR2 having the amino acid sequence of SEQ ID NO:89
   HC-CDR3 having the amino acid sequence of SEQ ID NO:90,
   or a variant thereof in which one or two or three amino acids in one or more of HC-CDR1, HC-CDR2, or HC-CDR3 are substituted with another amino acid.
(10) (4M2-D9) a VH region incorporating the following CDRs:
   HC-CDR1 having the amino acid sequence of SEQ ID NO:33
   HC-CDR2 having the amino acid sequence of SEQ ID NO:107
   HC-CDR3 having the amino acid sequence of SEQ ID NO:108,
   or a variant thereof in which one or two or three amino acids in one or more of HC-CDR1, HC-CDR2, or HC-CDR3 are substituted with another amino acid.
(11) (1M2-D2) a VH region incorporating the following CDRs:
   HC-CDR1 having the amino acid sequence of SEQ ID NO:120
   HC-CDR2 having the amino acid sequence of SEQ ID NO:121
   HC-CDR3 having the amino acid sequence of SEQ ID NO:122,
   or a variant thereof in which one or two or three amino acids in one or more of HC-CDR1, HC-CDR2, or HC-CDR3 are substituted with another amino acid.
(12) (4M2-D5) a VH region incorporating the following CDRs:
   HC-CDR1 having the amino acid sequence of SEQ ID NO:144
   HC-CDR2 having the amino acid sequence of SEQ ID NO:145
   HC-CDR3 having the amino acid sequence of SEQ ID NO:146,
   or a variant thereof in which one or two or three amino acids in one or more of HC-CDR1, HC-CDR2, or HC-CDR3 are substituted with another amino acid.
(13) (4M2-A8) a VH region incorporating the following CDRs:
   HC-CDR1 having the amino acid sequence of SEQ ID NO:158
   HC-CDR2 having the amino acid sequence of SEQ ID NO:159
   HC-CDR3 having the amino acid sequence of SEQ ID NO:160,
   or a variant thereof in which one or two or three amino acids in one or more of HC-CDR1, HC-CDR2, or HC-CDR3 are substituted with another amino acid.
(14) (9M2-C12) a VH region incorporating the following CDRs:
   HC-CDR1 having the amino acid sequence of SEQ ID NO:169
   HC-CDR2 having the amino acid sequence of SEQ ID NO:170
   HC-CDR3 having the amino acid sequence of SEQ ID NO:171,
   or a variant thereof in which one or two or three amino acids in one or more of HC-CDR1, HC-CDR2, or HC-CDR3 are substituted with another amino acid.
(15) (13D5 derived) a VH region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:246,
   or a variant thereof in which one or two or three amino acids in one or more of HC-CDR1, HC-CDR2, or HC-CDR3 are substituted with another amino acid.
(16) (13D5p) a VH region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:185,
   or a variant thereof in which one or two or three amino acids in one or more of HC-CDR1, HC-CDR2, or HC-CDR3 are substituted with another amino acid.
(17) (13D5-1) a VH region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:195,
   or a variant thereof in which one or two or three amino acids in one or more of HC-CDR1, HC-CDR2, or HC-CDR3 are substituted with another amino acid.
(18) (13D5-13) a VH region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:200,
   or a variant thereof in which one or two or three amino acids in one or more of HC-CDR1, HC-CDR2, or HC-CDR3 are substituted with another amino acid.

In some embodiments the antigen-binding molecule comprises a VH region according to one of (19) to (35) below:
(19) (V4-C24, V4-C26, V4-C27, V4-C28, V4-C30, V4-C31) a VH region incorporating the following FRs:
   HC-FR1 having the amino acid sequence of SEQ ID NO:63
   HC-FR2 having the amino acid sequence of SEQ ID NO:292
   HC-FR3 having the amino acid sequence of SEQ ID NO:293
   HC-FR4 having the amino acid sequence of SEQ ID NO:281,
   or a variant thereof in which one or two or three amino acids in one or more of HC-FR1, HC-FR2, HC-FR3, or HC-FR4 are substituted with another amino acid.
(20) (V4-C1, V4-C9) a VH region incorporating the following FRs:
   HC-FR1 having the amino acid sequence of SEQ ID NO:63
   HC-FR2 having the amino acid sequence of SEQ ID NO:279
   HC-FR3 having the amino acid sequence of SEQ ID NO:280
   HC-FR4 having the amino acid sequence of SEQ ID NO:281,
   or a variant thereof in which one or two or three amino acids in one or more of HC-FR1, HC-FR2, HC-FR3, or HC-FR4 are substituted with another amino acid.
(21) (4M2-C12) a VH region incorporating the following FRs:
   HC-FR1 having the amino acid sequence of SEQ ID NO:36
   HC-FR2 having the amino acid sequence of SEQ ID NO:37
   HC-FR3 having the amino acid sequence of SEQ ID NO:38
   HC-FR4 having the amino acid sequence of SEQ ID NO:39,
   or a variant thereof in which one or two or three amino acids in one or more of HC-FR1, HC-FR2, HC-FR3, or HC-FR4 are substituted with another amino acid.
(22) (4M2-B4) a VH region incorporating the following FRs:
   HC-FR1 having the amino acid sequence of SEQ ID NO:49
   HC-FR2 having the amino acid sequence of SEQ ID NO:37
   HC-FR3 having the amino acid sequence of SEQ ID NO:38
   HC-FR4 having the amino acid sequence of SEQ ID NO:39,
   or a variant thereof in which one or two or three amino acids in one or more of HC-FR1, HC-FR2, HC-FR3, or HC-FR4 are substituted with another amino acid.
(23) (V4H1) a VH region incorporating the following FRs:
   HC-FR1 having the amino acid sequence of SEQ ID NO:54
   HC-FR2 having the amino acid sequence of SEQ ID NO:55
   HC-FR3 having the amino acid sequence of SEQ ID NO:56
   HC-FR4 having the amino acid sequence of SEQ ID NO:39,
   or a variant thereof in which one or two or three amino acids in one or more of HC-FR1, HC-FR2, HC-FR3, or HC-FR4 are substituted with another amino acid.
(24) (V4H2) a VH region incorporating the following FRs:
   HC-FR1 having the amino acid sequence of SEQ ID NO:63
   HC-FR2 having the amino acid sequence of SEQ ID NO:64
   HC-FR3 having the amino acid sequence of SEQ ID NO:65
   HC-FR4 having the amino acid sequence of SEQ ID NO:39,
   or a variant thereof in which one or two or three amino acids in one or more of HC-FR1, HC-FR2, HC-FR3, or HC-FR4 are substituted with another amino acid.
(25) (2M1-B12) a VH region incorporating the following FRs:
   HC-FR1 having the amino acid sequence of SEQ ID NO:75
   HC-FR2 having the amino acid sequence of SEQ ID NO:76
   HC-FR3 having the amino acid sequence of SEQ ID NO:77
   HC-FR4 having the amino acid sequence of SEQ ID NO:78,
   or a variant thereof in which one or two or three amino acids in one or more of HC-FR1, HC-FR2, HC-FR3, or HC-FR4 are substituted with another amino acid.
(26) (4M2-C9) a VH region incorporating the following FRs:
   HC-FR1 having the amino acid sequence of SEQ ID NO:91
   HC-FR2 having the amino acid sequence of SEQ ID NO:92
   HC-FR3 having the amino acid sequence of SEQ ID NO:93
   HC-FR4 having the amino acid sequence of SEQ ID NO:94,
   or a variant thereof in which one or two or three amino acids in one or more of HC-FR1, HC-FR2, HC-FR3, or HC-FR4 are substituted with another amino acid.
(27) (2M1-D2) a VH region incorporating the following FRs:
   HC-FR1 having the amino acid sequence of SEQ ID NO:103
   HC-FR2 having the amino acid sequence of SEQ ID NO:76
   HC-FR3 having the amino acid sequence of SEQ ID NO:77
   HC-FR4 having the amino acid sequence of SEQ ID NO:78,
   or a variant thereof in which one or two or three amino acids in one or more of HC-FR1, HC-FR2, HC-FR3, or HC-FR4 are substituted with another amino acid.
(28) (4M2-D9) a VH region incorporating the following FRs:
   HC-FR1 having the amino acid sequence of SEQ ID NO:109
   HC-FR2 having the amino acid sequence of SEQ ID NO:110
   HC-FR3 having the amino acid sequence of SEQ ID NO:111
   HC-FR4 having the amino acid sequence of SEQ ID NO:112,
   or a variant thereof in which one or two or three amino acids in one or more of HC-FR1, HC-FR2, HC-FR3, or HC-FR4 are substituted with another amino acid.
(29) (1M2-D2) a VH region incorporating the following FRs:
   HC-FR1 having the amino acid sequence of SEQ ID NO:123
   HC-FR2 having the amino acid sequence of SEQ ID NO:124
   HC-FR3 having the amino acid sequence of SEQ ID NO:125
   HC-FR4 having the amino acid sequence of SEQ ID NO:78,
   or a variant thereof in which one or two or three amino acids in one or more of HC-FR1, HC-FR2, HC-FR3, or HC-FR4 are substituted with another amino acid.
(30) (5M1-A11) a VH region incorporating the following FRs:
   HC-FR1 having the amino acid sequence of SEQ ID NO:134
   HC-FR2 having the amino acid sequence of SEQ ID NO:92
   HC-FR3 having the amino acid sequence of SEQ ID NO:93
   HC-FR4 having the amino acid sequence of SEQ ID NO:135,
   or a variant thereof in which one or two or three amino acids in one or more of HC-FR1, HC-FR2, HC-FR3, or HC-FR4 are substituted with another amino acid.
(31) (4M2-D5) a VH region incorporating the following FRs:
   HC-FR1 having the amino acid sequence of SEQ ID NO:147
   HC-FR2 having the amino acid sequence of SEQ ID NO:148
   HC-FR3 having the amino acid sequence of SEQ ID NO:149
   HC-FR4 having the amino acid sequence of SEQ ID NO:135,
   or a variant thereof in which one or two or three amino acids in one or more of HC-FR1, HC-FR2, HC-FR3, or HC-FR4 are substituted with another amino acid.
(32) (4M2-A8) a VH region incorporating the following FRs:
   HC-FR1 having the amino acid sequence of SEQ ID NO:161
   HC-FR2 having the amino acid sequence of SEQ ID NO:162
   HC-FR3 having the amino acid sequence of SEQ ID NO:163
   HC-FR4 having the amino acid sequence of SEQ ID NO:135,
   or a variant thereof in which one or two or three amino acids in one or more of HC-FR1, HC-FR2, HC-FR3, or HC-FR4 are substituted with another amino acid.
(33) (9M2-C12) a VH region incorporating the following FRs:
   HC-FR1 having the amino acid sequence of SEQ ID NO:172
   HC-FR2 having the amino acid sequence of SEQ ID NO:173
   HC-FR3 having the amino acid sequence of SEQ ID NO:174
   HC-FR4 having the amino acid sequence of SEQ ID NO:175,
   or a variant thereof in which one or two or three amino acids in one or more of HC-FR1, HC-FR2, HC-FR3, or HC-FR4 are substituted with another amino acid.
(34) (13D5p, 13D5-1) a VH region incorporating the following FRs:
   HC-FR1 having the amino acid sequence of SEQ ID NO:103
   HC-FR2 having the amino acid sequence of SEQ ID NO:186
   HC-FR3 having the amino acid sequence of SEQ ID NO:187
   HC-FR4 having the amino acid sequence of SEQ ID NO:86,
   or a variant thereof in which one or two or three amino acids in one or more of HC-FR1, HC-FR2, HC-FR3, or HC-FR4 are substituted with another amino acid.
(35) (13D5-13) a VH region incorporating the following FRs:
   HC-FR1 having the amino acid sequence of SEQ ID NO:103
   HC-FR2 having the amino acid sequence of SEQ ID NO:186
   HC-FR3 having the amino acid sequence of SEQ ID NO:201
   HC-FR4 having the amino acid sequence of SEQ ID NO:86,
   or a variant thereof in which one or two or three amino acids in one or more of HC-FR1, HC-FR2, HC-FR3, or HC-FR4 are substituted with another amino acid.

In some embodiments the antigen-binding molecule comprises a VH region comprising the CDRs according to one of (1) to (18) above, and the FRs according to one of (19) to (35) above.

In some embodiments the antigen-binding molecule comprises a VH region according to one of (36) to (57) below:
(36) a VH region comprising the CDRs according to (1) and the FRs according to (19), (20), (21), (22), (23) or (24).
(37) a VH region comprising the CDRs according to (2) and the FRs according to (19).
(38) a VH region comprising the CDRs according to (3) and the FRs according to (20).
(39) a VH region comprising the CDRs according to (4) and the FRs according to (20).
(40) a VH region comprising the CDRs according to (5) and the FRs according to (21), (22), (23) or (24).
(41) a VH region comprising the CDRs according to (6) and the FRs according to (21).
(42) a VH region comprising the CDRs according to (6) and the FRs according to (22).
(43) a VH region comprising the CDRs according to (6) and the FRs according to (24).
(44) a VH region comprising the CDRs according to (7) and the FRs according to (23).
(45) a VH region comprising the CDRs according to (8) and the FRs according to (25).
(46) a VH region comprising the CDRs according to (8) and the FRs according to (27).
(47) a VH region comprising the CDRs according to (9) and the FRs according to (26).
(48) a VH region comprising the CDRs according to (9) and the FRs according to (30).
(49) a VH region comprising the CDRs according to (10) and the FRs according to (28).
(50) a VH region comprising the CDRs according to (11) and the FRs according to (29).
(51) a VH region comprising the CDRs according to (12) and the FRs according to (31).
(52) a VH region comprising the CDRs according to (13) and the FRs according to (32).
(53) a VH region comprising the CDRs according to (14) and the FRs according to (33).
(54) a VH region comprising the CDRs according to (15) and the FRs according to (34) or (35).
(55) a VH region comprising the CDRs according to (16) and the FRs according to (34).
(56) a VH region comprising the CDRs according to (17) and the FRs according to (34).
(57) a VH region comprising the CDRs according to (18) and the FRs according to (35).

In some embodiments the antigen-binding molecule comprises a VH region according to one of (58) to (76) below:
(58) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:276.
(59) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:285.
(60) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:289.
(61) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:32.
(62) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:48.
(63) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:52.
(64) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:62.
(65) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:71.
(66) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:87.
(67) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:102.
(68) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:106.
(69) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:119.
(70) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:133.
(71) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:143.
(72) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:157.
(73) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:168.
(74) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:183.
(75) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:194.
(76) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:199.

In some embodiments the antigen-binding molecule comprises a VL region according to one of (77) to (96) below:
(77) (4M2-C12 derived consensus) a VL region incorporating the following CDRs:
   LC-CDR1 having the amino acid sequence of SEQ ID NO:41
   LC-CDR2 having the amino acid sequence of SEQ ID NO:308
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(78) (C24/C26/C27 consensus) a VL region incorporating the following CDRs:
   LC-CDR1 having the amino acid sequence of SEQ ID NO:41
   LC-CDR2 having the amino acid sequence of SEQ ID NO:309
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(79) (V4-C24, V4-C26) a VL region incorporating the following CDRs:
   LC-CDR1 having the amino acid sequence of SEQ ID NO:41
   LC-CDR2 having the amino acid sequence of SEQ ID NO:295
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(80) (V4-C27, V4-C30, V4-C31) a VL region incorporating the following CDRs:
   LC-CDR1 having the amino acid sequence of SEQ ID NO:41
   LC-CDR2 having the amino acid sequence of SEQ ID NO:300
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(81) (4M2-C12/V4H1/V4H2 consensus) a VL region incorporating the following CDRs:
   LC-CDR1 having the amino acid sequence of SEQ ID NO:41
   LC-CDR2 having the amino acid sequence of SEQ ID NO:245
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(82) (4M2-C12, 4M2-B4, V4-C1, V4-C9, V4-C28) a VL region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(83) (V4H1) a VL region incorporating the following CDRs:
   LC-CDR1 having the amino acid sequence of SEQ ID NO:41
   LC-CDR2 having the amino acid sequence of SEQ ID NO:58
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(84) (V4H2) a VL region incorporating the following CDRs:
   LC-CDR1 having the amino acid sequence of SEQ ID NO:41
   LC-CDR2 having the amino acid sequence of SEQ ID NO:67
   LC-CDR3 having the amino acid sequence of SEQ ID NO:43;
   or a variant thereof in which one or two or three amino acids in one or more of LC-CDR1, LC-CDR2 or LC-CDR3 are substituted with another amino acid.
(85) (2M1-B12, 2M1-D2) a VL region incorporating the following CDRs:
   LC-CDR1 having the amino acid sequence of SEQ ID NO:80
   LC-CDR2 having the amino acid sequence of SEQ ID NO:81
   LC-CDR3 having the amino acid sequence of SEQ ID NO:82;
   or a variant thereof in which one or two or three amino acids in one or more of LC-CDR1, LC-CDR2 or LC-CDR3 are substituted with another amino acid.
(86) (4M2-C9) a VL region incorporating the following CDRs:
   LC-CDR1 having the amino acid sequence of SEQ ID NO:96
   LC-CDR2 having the amino acid sequence of SEQ ID NO:97
   LC-CDR3 having the amino acid sequence of SEQ ID NO:98;
   or a variant thereof in which one or two or three amino acids in one or more of LC-CDR1, LC-CDR2 or LC-CDR3 are substituted with another amino acid.
(87) (4M2-D9) a VH region incorporating the following CDRs:
   LC-CDR1 having the amino acid sequence of SEQ ID NO:114
   LC-CDR2 having the amino acid sequence of SEQ ID NO:67
   LC-CDR3 having the amino acid sequence of SEQ ID NO:115,
   or a variant thereof in which one or two or three amino acids in one or more of LC-CDR1, LC-CDR2, or LC-CDR3 are substituted with another amino acid.
(88) (1M2-D2) a VL region incorporating the following CDRs:
   LC-CDR1 having the amino acid sequence of SEQ ID NO:127
   LC-CDR2 having the amino acid sequence of SEQ ID NO:128
   LC-CDR3 having the amino acid sequence of SEQ ID NO:129;
   or a variant thereof in which one or two or three amino acids in one or more of LC-CDR1, LC-CDR2 or LC-CDR3 are substituted with another amino acid.
(89) (5M1-A11) a VL region incorporating the following CDRs:
   LC-CDR1 having the amino acid sequence of SEQ ID NO:137
   LC-CDR2 having the amino acid sequence of SEQ ID NO:138
   LC-CDR3 having the amino acid sequence of SEQ ID NO:139;
   or a variant thereof in which one or two or three amino acids in one or more of LC-CDR1, LC-CDR2 or LC-CDR3 are substituted with another amino acid.
(90) (4M2-D5) a VL region incorporating the following CDRs:
   LC-CDR1 having the amino acid sequence of SEQ ID NO:151
   LC-CDR2 having the amino acid sequence of SEQ ID NO:152
   LC-CDR3 having the amino acid sequence of SEQ ID NO:153;
   or a variant thereof in which one or two or three amino acids in one or more of LC-CDR1, LC-CDR2 or LC-CDR3 are substituted with another amino acid.
(91) (4M2-A8) a VL region incorporating the following CDRs:
   LC-CDR1 having the amino acid sequence of SEQ ID NO:165
   LC-CDR2 having the amino acid sequence of SEQ ID NO:152
   LC-CDR3 having the amino acid sequence of SEQ ID NO:153;
   or a variant thereof in which one or two or three amino acids in one or more of LC-CDR1, LC-CDR2 or LC-CDR3 are substituted with another amino acid.
(92) (9M2-C12) a VL region incorporating the following CDRs:
   LC-CDR1 having the amino acid sequence of SEQ ID NO:177
   LC-CDR2 having the amino acid sequence of SEQ ID NO:178
   LC-CDR3 having the amino acid sequence of SEQ ID NO:179;
   or a variant thereof in which one or two or three amino acids in one or more of LC-CDR1, LC-CDR2 or LC-CDR3 are substituted with another amino acid.
(93) (13D5p derived) a VL region incorporating the following CDRs:
   LC-CDR1 having the amino acid sequence of SEQ ID NO:247
   LC-CDR2 having the amino acid sequence of SEQ ID NO:178
   LC-CDR3 having the amino acid sequence of SEQ ID NO:190;
   or a variant thereof in which one or two or three amino acids in one or more of LC-CDR1, LC-CDR2 or LC-CDR3 are substituted with another amino acid.
(94) (13D5p) a VL region incorporating the following CDRs:
   LC-CDR1 having the amino acid sequence of SEQ ID NO:189
   LC-CDR2 having the amino acid sequence of SEQ ID NO:178
   LC-CDR3 having the amino acid sequence of SEQ ID NO:190;
   or a variant thereof in which one or two or three amino acids in one or more of LC-CDR1, LC-CDR2 or LC-CDR3 are substituted with another amino acid.
(95) (13D5-1) a VL region incorporating the following CDRs:
   LC-CDR1 having the amino acid sequence of SEQ ID NO:197
   LC-CDR2 having the amino acid sequence of SEQ ID NO:178
   LC-CDR3 having the amino acid sequence of SEQ ID NO:190;
   or a variant thereof in which one or two or three amino acids in one or more of LC-CDR1, LC-CDR2 or LC-CDR3 are substituted with another amino acid.
(96) (13D5-13) a VL region incorporating the following CDRs:
   LC-CDR1 having the amino acid sequence of SEQ ID NO:203
   LC-CDR2 having the amino acid sequence of SEQ ID NO:178
   LC-CDR3 having the amino acid sequence of SEQ ID NO:190;
   or a variant thereof in which one or two or three amino acids in one or more of LC-CDR1, LC-CDR2 or LC-CDR3 are substituted with another amino acid.

In some embodiments the antigen-binding molecule comprises a VL region according to one of (97) to (120) below:
(97) (V4-C1) a VL region incorporating the following FRs:
   LC-FR1 having the amino acid sequence of SEQ ID NO:59
   LC-FR2 having the amino acid sequence of SEQ ID NO:283
   LC-FR3 having the amino acid sequence of SEQ ID NO:284
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(98) (V4-C9) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:284
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(99) (V4-C24) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:296
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(100) (V4-C26) a VL region incorporating the following FRs:
   LC-FR1 having the amino acid sequence of SEQ ID NO:288
   LC-FR2 having the amino acid sequence of SEQ ID NO:298
   LC-FR3 having the amino acid sequence of SEQ ID NO:284
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(101) (V4-C27) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:284
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(102) (V4-C28) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:296
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(103) (V4-C30) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:296
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(104) (V4-C31) a VL region incorporating the following FRs:
   LC-FR1 having the amino acid sequence of SEQ ID NO:288
   LC-FR2 having the amino acid sequence of SEQ ID NO:283
   LC-FR3 having the amino acid sequence of SEQ ID NO:304
   LC-FR4 having the amino acid sequence of SEQ ID NO:47,
or a variant thereof in which one or two or three amino acids in one or more of LC-FR1, LC-FR2, LC-FR3, or LC-FR4 are substituted with another amino acid.
(105) (4M2-C12) a VL region incorporating the following FRs:
   LC-FR1 having the amino acid sequence of SEQ ID NO:44
   LC-FR2 having the amino acid sequence of SEQ ID NO:45
   LC-FR3 having the amino acid sequence of SEQ ID NO:46
   LC-FR4 having the amino acid sequence of SEQ ID NO:47,
   or a variant thereof in which one or two or three amino acids in one or more of LC-FR1, LC-FR2, LC-FR3, or LC-FR4 are substituted with another amino acid.
(106) (4M2-B4) a VL region incorporating the following FRs:
   LC-FR1 having the amino acid sequence of SEQ ID NO:51
   LC-FR2 having the amino acid sequence of SEQ ID NO:45
   LC-FR3 having the amino acid sequence of SEQ ID NO:46
   LC-FR4 having the amino acid sequence of SEQ ID NO:47,
   or a variant thereof in which one or two or three amino acids in one or more of LC-FR1, LC-FR2, LC-FR3, or LC-FR4 are substituted with another amino acid.
(107) (V4H1) a VL region incorporating the following FRs:
   LC-FR1 having the amino acid sequence of SEQ ID NO:59
   LC-FR2 having the amino acid sequence of SEQ ID NO:60
   LC-FR3 having the amino acid sequence of SEQ ID NO:61
   LC-FR4 having the amino acid sequence of SEQ ID NO:47,
   or a variant thereof in which one or two or three amino acids in one or more of LC-FR1, LC-FR2, LC-FR3, or LC-FR4 are substituted with another amino acid.
(108) (V4H2) a VL region incorporating the following FRs:
   LC-FR1 having the amino acid sequence of SEQ ID NO:68
   LC-FR2 having the amino acid sequence of SEQ ID NO:69
   LC-FR3 having the amino acid sequence of SEQ ID NO:70
   LC-FR4 having the amino acid sequence of SEQ ID NO:47,
   or a variant thereof in which one or two or three amino acids in one or more of LC-FR1, LC-FR2, LC-FR3, or LC-FR4 are substituted with another amino acid.
(109) (2M1-B12) a VL region incorporating the following FRs:
   LC-FR1 having the amino acid sequence of SEQ ID NO:83
   LC-FR2 having the amino acid sequence of SEQ ID NO:84
   LC-FR3 having the amino acid sequence of SEQ ID NO:85
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(110) (4M2-C9) a VL region incorporating the following FRs:
   LC-FR1 having the amino acid sequence of SEQ ID NO:99
   LC-FR2 having the amino acid sequence of SEQ ID NO:100
   LC-FR3 having the amino acid sequence of SEQ ID NO:101
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(111) (2M1-D2) a VL region incorporating the following FRs:
   LC-FR1 having the amino acid sequence of SEQ ID NO:105
   LC-FR2 having the amino acid sequence of SEQ ID NO:84
   LC-FR3 having the amino acid sequence of SEQ ID NO:85
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(112) (4M2-D9) a VL region incorporating the following FRs:
   LC-FR1 having the amino acid sequence of SEQ ID NO:116
   LC-FR2 having the amino acid sequence of SEQ ID NO:117
   LC-FR3 having the amino acid sequence of SEQ ID NO:118
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(113) (1M2-D2) a VL region incorporating the following FRs:
   LC-FR1 having the amino acid sequence of SEQ ID NO:130
   LC-FR2 having the amino acid sequence of SEQ ID NO:131
   LC-FR3 having the amino acid sequence of SEQ ID NO:132
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(114) (5M1-A11) a VL region incorporating the following FRs:
   LC-FR1 having the amino acid sequence of SEQ ID NO:140
   LC-FR2 having the amino acid sequence of SEQ ID NO:141
   LC-FR3 having the amino acid sequence of SEQ ID NO:142
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(115) (4M2-D5) a VL region incorporating the following FRs:
   LC-FR1 having the amino acid sequence of SEQ ID NO:154
   LC-FR2 having the amino acid sequence of SEQ ID NO:155
   LC-FR3 having the amino acid sequence of SEQ ID NO:156
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(116) (4M2-A8) a VL region incorporating the following FRs:
   LC-FR1 having the amino acid sequence of SEQ ID NO:166
   LC-FR2 having the amino acid sequence of SEQ ID NO:155
   LC-FR3 having the amino acid sequence of SEQ ID NO:167
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(117) (9M2-C12) a VL region incorporating the following FRs:
   LC-FR1 having the amino acid sequence of SEQ ID NO:180
   LC-FR2 having the amino acid sequence of SEQ ID NO:181
   LC-FR3 having the amino acid sequence of SEQ ID NO:182
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(118) (13D5p) a VL region incorporating the following FRs:
   LC-FR1 having the amino acid sequence of SEQ ID NO:191
   LC-FR2 having the amino acid sequence of SEQ ID NO:192
   LC-FR3 having the amino acid sequence of SEQ ID NO:193
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(119) (13D5-1) a VL region incorporating the following FRs:
   LC-FR1 having the amino acid sequence of SEQ ID NO:191
   LC-FR2 having the amino acid sequence of SEQ ID NO:198
   LC-FR3 having the amino acid sequence of SEQ ID NO:193
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.
(120) (13D5-13) a VL region incorporating the following FRs:
   LC-FR1 having the amino acid sequence of SEQ ID NO:191
   LC-FR2 having the amino acid sequence of SEQ ID NO:192
   LC-FR3 having the amino acid sequence of SEQ ID NO:204
   LC-FR4 having the amino acid sequence of SEQ ID NO:86,
   or a variant thereof in which one or two or three amino acids in one or more of LC-FR1, LC-FR2, LC-FR3, or LC-FR4 are substituted with another amino acid.

In some embodiments the antigen-binding molecule comprises a VL region comprising the CDRs according to one of (77) to (96) above, and the FRs according to one of (97) to (120) above.

In some embodiments the antigen-binding molecule comprises a VL region according to one of (121) to (148) below:
(121) a VL region comprising the CDRs according to (77) and the FRs according to (97), (98), (99), (100), (101), (102), (103), (104), (105), (106), (107) or (108).
(122) a VL region comprising the CDRs according to (78) and the FRs according to (99), (100) or (101).
(123) a VL region comprising the CDRs according to (79) and the FRs according to (99).
(124) a VL region comprising the CDRs according to (79) and the FRs according to (100).
(125) a VL region comprising the CDRs according to (80) and the FRs according to (101).
(126) a VL region comprising the CDRs according to (82) and the FRs according to (97).
(127) a VL region comprising the CDRs according to (82) and the FRs according to (98).
(128) a VL region comprising the CDRs according to (82) and the FRs according to (102).
(129) a VL region comprising the CDRs according to (80) and the FRs according to (103).
(130) a VL region comprising the CDRs according to (80) and the FRs according to (104).
(131) a VL region comprising the CDRs according to (81) and the FRs according to (105), (106), (107) or (108).
(132) a VL region comprising the CDRs according to (82) and the FRs according to (105).
(133) a VL region comprising the CDRs according to (82) and the FRs according to (106).
(134) a VL region comprising the CDRs according to (83) and the FRs according to (107).
(135) a VL region comprising the CDRs according to (84) and the FRs according to (108).
(136) a VL region comprising the CDRs according to (85) and the FRs according to (109).
(137) a VL region comprising the CDRs according to (85) and the FRs according to (111).
(138) a VL region comprising the CDRs according to (86) and the FRs according to (110).
(139) a VL region comprising the CDRs according to (87) and the FRs according to (112).
(140) a VL region comprising the CDRs according to (88) and the FRs according to (113).
(141) a VL region comprising the CDRs according to (89) and the FRs according to (114).
(142) a VL region comprising the CDRs according to (90) and the FRs according to (115).
(143) a VL region comprising the CDRs according to (91) and the FRs according to (116).
(144) a VL region comprising the CDRs according to (92) and the FRs according to (117).
(145) a VL region comprising the CDRs according to (93) and the FRs according to (118), (119) or (120).
(146) a VL region comprising the CDRs according to (94) and the FRs according to (118).
(147) a VL region comprising the CDRs according to (95) and the FRs according to (119).
(148) a VL region comprising the CDRs according to (96) and the FRs according to (120).

In some embodiments the antigen-binding molecule comprises a VL region according to one of (149) to (173) below:
(149) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:310.
(150) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:282.
(151) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:287.
(152) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:294.
(153) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:297.
(154) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:299.
(155) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:301.
(156) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:302.
(157) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:303.
(158) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:40.
(159) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:50.
(160) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:57.
(161) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:66.
(162) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:79.
(163) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:95.
(164) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:104.
(165) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:113.
(166) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:126.
(167) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:136.
(168) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:150.
(169) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:164.
(170) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:176.
(171) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:188.
(172) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:196.
(173) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:202.

In some embodiments the antigen-binding molecule comprises a VH region according to any one of (1) to (76) above, and a VL region according to any one of (77) to (173) above.

In some embodiments, the antigen-binding molecule comprises:
a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:289; and
a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:297.

In some embodiments, the antigen-binding molecule comprises, or consists of:
(i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:315; and
(ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:317.

In some embodiments, the antigen-binding molecule comprises, or consists of:
(i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:331; and
(ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:317.

In embodiments in accordance with the present invention in which one or more amino acids are substituted with another amino acid, the substitutions may be conservative substitutions, for example according to the following Table. In some embodiments, amino acids in the same block in the middle column are substituted. In some embodiments, amino acids in the same line in the rightmost column are substituted:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In some embodiments, substitution(s) may be functionally conservative. That is, in some embodiments the substitution may not affect (or may not substantially affect) one or more functional properties (e.g. target binding) of the antigen-binding molecule comprising the substitution as compared to the equivalent unsubstituted molecule.

The VH and VL region of an antigen-binding region of an antibody together constitute the Fv region. In some embodiments, the antigen-binding molecule according to the present invention comprises, or consists of, an Fv region which binds to VISTA. In some embodiments the VH and VL regions of the Fv are provided as single polypeptide joined by a linker region, i.e. a single chain Fv (scFv).

In some embodiments the antigen-binding molecule of the present invention comprises one or more regions of an immunoglobulin heavy chain constant sequence. In some embodiments the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM.

In some embodiments the immunoglobulin heavy chain constant sequence is human immunoglobulin G 1 constant (IGHG1; UniProt: P01857-1, v1; SEQ ID NO:205). Positions 1 to 98 of SEQ ID NO:205 form the CH1 region (SEQ ID NO:206). Positions 99 to 110 of SEQ ID NO:205 form a hinge region between CH1 and CH2 regions (SEQ ID NO:207). Positions 111 to 223 of SEQ ID NO:205 form the CH2 region (SEQ ID NO: 208). Positions 224 to 330 of SEQ ID NO:205 form the CH3 region (SEQ ID NO:209).

The exemplified antigen-binding molecules may be prepared using pFUSE-CHIg-hG1, which comprises the substitutions D356E, L358M (positions numbered according to EU numbering) in the CH3 region. The amino acid sequence of the CH3 region encoded by pFUSE-CHIg-hG1 is shown in SEQ ID NO:210. It will be appreciated that CH3 regions may be provided with further substitutions in accordance with modification to an Fc region of the antigen-binding molecule as described herein.

In some embodiments a CH1 region comprises or consists of the sequence of SEQ ID NO:206, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO: 206. In some embodiments a CH1-CH2 hinge region comprises or consists of the sequence of SEQ ID NO: 207, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:207. In some embodiments a CH2 region comprises or consists of the sequence of SEQ ID NO:208, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:208. In some embodiments a CH3 region comprises or consists of the sequence of SEQ ID NO:209 or 210, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO:209 or 210.

In some embodiments the antigen-binding molecule of the present invention comprises one or more regions of an immunoglobulin light chain constant sequence. In some embodiments the immunoglobulin light chain constant sequence is human immunoglobulin kappa constant (IGKC; Cκ; UniProt: P01834-1, v2; SEQ ID NO:211). In some embodiments the immunoglobulin light chain constant sequence is a human immunoglobulin lambda constant (IGLC; Cλ), e.g. IGLC1, IGLC2, IGLC3, IGLC6 or IGLC7. In some embodiments a CL region comprises or consists of the sequence of SEQ ID NO:211, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO: 211.

The VL and light chain constant (CL) region, and the VH region and heavy chain constant 1 (CH1) region of an antigen-binding region of an antibody together constitute the Fab region. In some embodiments the antigen-binding molecule comprises a Fab region comprising a VH, a CH1, a VL and a CL (e.g. Cκ or Cλ). In some embodiments the Fab region comprises a polypeptide comprising a VH and a CH1 (e.g. a VH-CH1 fusion polypeptide), and a polypeptide comprising a VL and a CL (e.g. a VL-CL fusion polypeptide). In some embodiments the Fab region comprises a polypeptide comprising a VH and a CL (e.g. a VH-CL fusion polypeptide) and a polypeptide comprising a VL and a CH (e.g. a VL-CH1 fusion polypeptide); that is, in some embodiments the Fab region is a CrossFab region. In some embodiments the VH, CH1, VL and CL regions of the Fab or CrossFab are provided as single polypeptide joined by linker regions, i.e. as a single chain Fab (scFab) or a single chain CrossFab (scCrossFab).

In some embodiments, the antigen-binding molecule of the present invention comprises, or consists of, a Fab region which binds to VISTA.

In some embodiments, the antigen-binding molecule described herein comprises, or consists of, a whole antibody which binds to VISTA. As used herein, “whole antibody” refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety.

Immunoglobulins of type G (i.e. IgG) are ˜150 kDa glycoproteins comprising two heavy chains and two light chains. From N- to C-terminus, the heavy chains comprise a VH followed by a heavy chain constant region comprising three constant domains (CH1, CH2, and CH3), and similarly the light chain comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments, the antigen-binding molecule described herein comprises, or consists of, an IgG (e.g. IgG1, IgG2, IgG3, IgG4), IgA (e.g. IgA1, IgA2), IgD, IgE, or IgM which binds to VISTA.

In some embodiments, the antigen-binding molecule of the present invention is at least monovalent binding for VISTA. Binding valency refers to the number of binding sites in an antigen-binding molecule for a given antigenic determinant. Accordingly, in some embodiments the antigen-binding molecule comprises at least one binding site for VISTA.

In some embodiments the antigen-binding molecule comprises more than one binding site for VISTA, e.g. 2, 3 or 4 binding sites. The binding sites may be the same or different. In some embodiments the antigen-binding molecule is e.g. bivalent, trivalent or tetravalent for VISTA.

Aspects of the present invention relate to multispecific antigen-binding molecules. By “multispecific” it is meant that the antigen-binding molecule displays specific binding to more than one target. In some embodiments the antigen-binding molecule is a bispecific antigen-binding molecule. In some embodiments the antigen-binding molecule comprises at least two different antigen-binding domains (i.e. at least two antigen-binding domains, e.g. comprising non-identical VHs and VLs).

In some embodiments the antigen-binding molecule binds to VISTA and another target (e.g. an antigen other than VISTA), and so is at least bispecific. The term “bispecific” means that the antigen-binding molecule is able to bind specifically to at least two distinct antigenic determinants.

It will be appreciated that an antigen-binding molecule according to the present invention (e.g. a multispecific antigen-binding molecule) may comprise antigen-binding molecules capable of binding to the targets for which the antigen-binding molecule is specific. For example, an antigen-binding molecule which is capable of binding to VISTA and an antigen other than VISTA may comprise: (i) an antigen-binding molecule which is capable of binding to VISTA, and (ii) an antigen-binding molecule which is capable of binding to an antigen other than VISTA.

It will also be appreciated that an antigen-binding molecule according to the present invention (e.g. a multispecific antigen-binding molecule) may comprise antigen-binding polypeptides or antigen-binding polypeptide complexes capable of binding to the targets for which the antigen-binding molecule is specific. For example, an antigen-binding molecule according to the invention may comprise e.g. (i) an antigen-binding polypeptide complex capable of binding to VISTA, comprising a light chain polypeptide (comprising the structure VL-CL) and a heavy chain polypeptide (comprising the structure VH-CH1-CH2-CH3), and (ii) an antigen-binding polypeptide complex capable of binding to an antigen other than VISTA, comprising a light chain polypeptide (comprising the structure VL-CL) and a heavy chain polypeptide (comprising the structure VH-CH1-CH2-CH3).

In some embodiments, a component antigen-binding molecule of a larger antigen-binding molecule (e.g. a multispecific antigen-biding molecule) may be referred to e.g. as an “antigen-binding domain” or “antigen-binding region” of the larger antigen-binding molecule.

In some embodiments the antigen-binding molecule comprises an antigen-binding molecule capable of binding to VISTA, and an antigen-binding molecule capable of binding to an antigen other than VISTA. In some embodiments, the antigen other than VISTA is an immune cell surface molecule. In some embodiments, the antigen other than VISTA is a cancer cell antigen. In some embodiments the antigen other than VISTA is a receptor molecule, e.g. a cell surface receptor. In some embodiments the antigen other than VISTA is a cell signalling molecule, e.g. a cytokine, chemokine, interferon, interleukin or lymphokine. In some embodiments the antigen other than VISTA is a growth factor or a hormone.

A cancer cell antigen is an antigen which is expressed or over-expressed by a cancer cell. A cancer cell antigen may be any peptide/polypeptide, glycoprotein, lipoprotein, glycan, glycolipid, lipid, or fragment thereof. A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by the antigen-binding molecule described herein is displayed on the external surface of the cancer cell (i.e. is extracellular). The cancer cell antigen may be a cancer-associated antigen. In some embodiments the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression of by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

An immune cell surface molecule may be any peptide/polypeptide, glycoprotein, lipoprotein, glycan, glycolipid, lipid, or fragment thereof expressed at or on the cell surface of an immune cell. In some embodiments, the part of the immune cell surface molecule which is bound by the antigen-binding molecule of the present invention is on the external surface of the immune cell (i.e. is extracellular). The immune cell surface molecule may be expressed at the cell surface of any immune cell. In some embodiments, the immune cell may be a cell of hematopoietic origin, e.g. a neutrophil, eosinophil, basophil, dendritic cell, lymphocyte, or monocyte. The lymphocyte may be e.g. a T cell, B cell, natural killer (NK) cell, NKT cell or innate lymphoid cell (ILC), or a precursor thereof (e.g. a thymocyte or pre-B cell). In some embodiments the immune cell surface molecule may be a costimulatory molecule (e.g. CD28, OX40, 4-1BB, ICOS or CD27) or a ligand thereof. In some embodiments the immune cell surface molecule may be a checkpoint molecule (e.g. PD-1, CTLA-4, LAG-3, TIM-3, TIGIT or BTLA) or a ligand thereof.

Multispecific antigen-binding molecules according to the invention may be provided in any suitable format, such as those formats described in described in Brinkmann and Kontermann MAbs (2017) 9(2): 182-212, which is hereby incorporated by reference in its entirety. Suitable formats include those shown in FIG. 2 of Brinkmann and Kontermann MAbs (2017) 9(2): 182-212: antibody conjugates, e.g. IgG₂, F(ab′)₂ or CovX-Body; IgG or IgG-like molecules, e.g. IgG, chimeric IgG, κλ-body common HC; CH1/CL fusion proteins, e.g. scFv2-CH1/CL, VHH2-CH1/CL; ‘variable domain only’ bispecific antigen-binding molecules, e.g. tandem scFv (taFV), triplebodies, diabodies (Db), dsDb, Db(kih), DART, scDB, dsFv-dsFv, tandAbs, triple heads, tandem dAb/VHH, tertravalent dAb. VHH; Non-Ig fusion proteins, e.g. scFv₂-albumin, scDb-albumin, taFv-albumin, taFv-toxin, miniantibody, DNL-Fab₂, DNL-Fab₂-scFv, DNL-Fab₂-IgG-cytokine₂, ImmTAC (TCR-scFv); modified Fc and CH3 fusion proteins, e.g. scFv-Fc(kih), scFv-Fc(CH3 charge pairs), scFv-Fc(EW-RVT), scFv-fc (HA-TF), scFv-Fc(SEEDbody), taFv-Fc(kih), scFv-Fc(kih)-Fv, Fab-Fc(kih)-scFv, Fab-scFv-Fc(kih), Fab-scFv-Fc(BEAT), Fab-scFv-Fc(SEEDbody), DART-Fc, scFv-CH3 (kih), TriFabs; Fc fusions, e.g. Di-diabody, scDb-Fc, taFv-Fc, scFv-Fc-scFv, HCAb-VHH, Fab-scFv-Fc, scFv₄-Ig, scFv₂-Fcab; CH3 fusions, e.g. Dia-diabody, scDb-CH3; IgE/IgM CH2 fusions, e.g. scFv-EHD2-scFv, scFvMHD2-scFv; Fab fusion proteins, e.g. Fab-scFv (bibody), Fab-scFv₂ (tribody), Fab-Fv, Fab-dsFv, Fab-VHH, orthogonal Fab-Fab; non-Ig fusion proteins, e.g. DNL-Fab₃, DNL-Fab₂-scFv, DNL-Fab₂-IgG-cytokine₂; asymmetric IgG or IgG-like molecules, e.g. IgG(kih), IgG(kih) common LC, ZW1 IgG common LC, Biclonics common LC, CrossMab, CrossMab(kih), scFab-IgG(kih), Fab-scFab-IgG(kih), orthogonal Fab IgG(kih), DuetMab, CH3 charge pairs+CH1/CL charge pairs, hinge/CH3 charge pairs, SEED-body, Duobody, four-in-one-CrossMab(kih), LUZ-Y common LC; LUZ-Y scFab-IgG, FcFc *; appended and Fc-modified IgGs, e.g. IgG(kih)-Fv, IgG HA-TF-Fv, IgG(kih) scFab, scFab-Fc(kih)-scFv2, scFab-Fc(kih)-scFv, half DVD-Ig, DVI-Ig (four-in-one), CrossMab-Fab; modified Fc and CH3 fusion proteins, e.g. Fab-Fc(kih)-scFv, Fab-scFv-Fc(kih), Fab-scFv-Fc(BEAT), Fab-scFv-Fc-SEEDbody, TriFab; appended IgGs—HC fusions, e.g. IgG-HC, scFv, IgG-dAb, IgG-taFV, IgG-CrossFab, IgG-orthogonal Fab, IgG-(CαCβ) Fab, scFv-HC-IgG, tandem Fab-IgG(orthogonal Fab) Fab-IgG(CαCβ Fab), Fab-IgG(CR3), Fab-hinge-IgG(CR3); appended IgGs-LC fusions, e.g. IgG-scFv (LC), scFv (LC)-IgG, dAb-IgG; appended IgGs—HC and LC fusions, e.g. DVD-Ig, TVD-Ig, CODV-Ig, scFv₄-IgG, Zybody; Fc fusions, e.g. Fab-scFv-Fc, scFv4-Ig; F(ab′)₂ fusions, e.g. F(ab′)₂-scFv2; CH1/CL fusion proteins e.g. scFv₂-CH1-hinge/CL; modified IgGs, e.g. DAF (two-in one-IgG), DutaMab, Mab²; and non-Ig fusions, e.g. DNL-Fab₄-IgG.

The skilled person is able to design and prepare bispecific antigen-binding molecules. Methods for producing bispecific antigen-binding molecules include chemically crosslinking of antigen-binding molecules or antibody fragments, e.g. with reducible disulphide or non-reducible thioether bonds, for example as described in Segal and Bast, 2001. Production of Bispecific Antigen-binding molecules. Current Protocols in Immunology. 14: IV: 2.13:2.13.1-2.13.16, which is hereby incorporated by reference in its entirety. For example, N-succinimidyl-3-(−2-pyridyldithio)-propionate (SPDP) can be used to chemically crosslink e.g. Fab fragments via hinge region SH-groups, to create disulfide-linked bispecific F(ab)₂ heterodimers.

Other methods for producing bispecific antigen-binding molecules include fusing antibody-producing hybridomas e.g. with polyethylene glycol, to produce a quadroma cell capable of secreting bispecific antibody, for example as described in D. M. and Bast, B. J. 2001. Production of Bispecific Antigen-binding molecules. Current Protocols in Immunology. 14: IV: 2.13:2.13.1-2.13.16.

Bispecific antigen-binding molecules according to the present invention can also be produced recombinantly, by expression from e.g. a nucleic acid construct encoding polypeptides for the antigen-binding molecules, for example as described in Antibody Engineering: Methods and Protocols, Second Edition (Humana Press, 2012), at Chapter 40: Production of Bispecific Antigen-binding molecules: Diabodies and Tandem scFv (Hornig and Färber-Schwarz), or French, How to make bispecific antigen-binding molecules, Methods Mol. Med. 2000; 40:333-339, the entire contents of both of which are hereby incorporated by reference. For example, a DNA construct encoding the light and heavy chain variable domains for the two antigen-binding fragments (i.e. the light and heavy chain variable domains for the antigen-binding fragment capable of binding VISTA, and the light and heavy chain variable domains for the antigen-binding fragment capable of binding to another target protein), and including sequences encoding a suitable linker or dimerization domain between the antigen-binding fragments can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

### Fc Regions

In some embodiments the antigen-binding molecules of the present invention comprise an Fc region.

In IgG IgA and IgD isotype Fc regions are composed of CH2 and CH3 regions from one polypeptide, and CH2 and CH3 regions from another polypeptide. The CH2 and CH3 regions from the two polypeptides together form the Fc region. In IgM and IgE isotypes the Fc regions contain three constant domains (CH2, CH3 and CH4), and CH2 to CH4 from the two polypeptides together form the Fc region.

Fc regions provide for interaction with Fc receptors and other molecules of the immune system to bring about functional effects. IgG Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, NK cells and T cells) through interaction between the Fc region and Fc receptors expressed by the immune cells, recruitment of complement pathway components through binding of the Fc region to complement protein C1q, and consequent activation of the complement cascade.

Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

Modifications to antibody Fc regions that influence Fc-mediated functions are known in the art, such as those described e.g. in Wang et al., Protein Cell (2018) 9(1): 63-73, which is hereby incorporated by reference in its entirety. In particular, exemplary Fc region modifications known to influence antibody effector function are summarised in Table 1 of Wang et al., Protein Cell (2018) 9(1): 63-73. Modifications to Fc regions which influence antibody effector activity are described hereinbelow.

Where an Fc region/CH2/CH3 is described as comprising modification(s) “corresponding to” reference substitution(s), equivalent substitution(s) in the homologous Fc/CH2/CH3 are contemplated. By way of illustration, L234A/L235A substitutions in human IgG1 (numbered according to the EU numbering system as described in Kabat et al., Sequences of Proteins of Immunological Interest, 5th Ed. Public Health Service, National Institutes of Health, Bethesda, MD, 1991) correspond to L to A substitutions at positions 117 and 118 of the mouse Ig gamma-2A chain C region, A allele, numbered according to SEQ ID NO: 256.

Where an Fc region is described as comprising a modification, the modification may be present in one or both of the polypeptide chains which together form the Fc region.

In some embodiments, the antigen-binding molecule of the present invention comprises an Fc region comprising modification. In some embodiments, the antigen-binding molecule of the present invention comprises an Fc region comprising modification in one or more of the CH2 and/or CH3 regions.

In some embodiments the Fc region comprises modification to increase an Fc-mediated function. In some embodiments the Fc region comprises modification to increase ADCC. In some embodiments the Fc region comprises modification to increase ADCP. In some embodiments the Fc region comprises modification to increase CDC. An antigen-binding molecule comprising an Fc region comprising modification to increase an Fc-mediated function (e.g. ADCC, ADCP, CDC) induces an increased level of the relevant effector function as compared to an antigen-binding molecule comprising the corresponding unmodified Fc region.

In some embodiments the Fc region comprises modification to increase binding to an Fc receptor. In some embodiments the Fc region comprises modification to increase binding to an Fcγ receptor. In some embodiments the Fc region comprises modification to increase binding to one or more of FcγRI, FcγRIIa, FcγRIIb, FcγRIIc, FcγRIIIa and FcγRIIIb. In some embodiments the Fc region comprises modification to increase binding to FcγRIIIa. In some embodiments the Fc region comprises modification to increase binding to FcγRIIa. In some embodiments the Fc region comprises modification to increase binding to FcγRIIb. In some embodiments the Fc region comprises modification to increase binding to FcRn. In some embodiments the Fc region comprises modification to increase binding to a complement protein. In some embodiments the Fc region comprises modification to increase binding to C1q. In some embodiments the Fc region comprises modification to promote hexamerisation of the antigen-binding molecule. In some embodiments the Fc region comprises modification to increase antigen-binding molecule half-life. In some embodiments the Fc region comprises modification to increase co-engagement.

In some embodiments the Fc region comprises modification corresponding to the combination of substitutions F243L/R292P/Y300L/V305l/P396L as described in Stavenhagen et al. Cancer Res. (2007) 67:8882-8890. In some embodiments the Fc region comprises modification corresponding to the combination of substitutions S239D/1332E or S239D/1332E/A330L as described in Lazar et al., Proc Natl Acad Sci USA. (2006) 103:4005-4010. In some embodiments the Fc region comprises modification corresponding to the combination of substitutions S298A/E333A/K334A as described in Shields et al., J Biol Chem. (2001) 276:6591-6604. In some embodiments the Fc region comprises modification to one of heavy chain polypeptides corresponding to the combination of substitutions L234Y/L235Q/G236W/S239M/H268D/D270E/S298A, and modification to the other heavy chain polypeptide corresponding to the combination of substitutions D270E/K326D/A330M/K334E, as described in Mimoto et al., MAbs. (2013): 5:229-236. In some embodiments the Fc region comprises modification corresponding to the combination of substitutions G236A/S239D/1332E as described in Richards et al., Mol Cancer Ther. (2008) 7:2517-2527.

In some embodiments the Fc region comprises modification corresponding to the combination of substitutions K326W/E333S as described in Idusogie et al. J Immunol. (2001) 166(4): 2571-5. In some embodiments the Fc region comprises modification corresponding to the combination of substitutions S267E/H268F/S324T as described in Moore et al. MAbs. (2010) 2(2): 181-9. In some embodiments the Fc region comprises modification corresponding to the combination of substitutions described in Natsume et al., Cancer Res. (2008) 68(10): 3863-72. In some embodiments the Fc region comprises modification corresponding to the combination of substitutions E345R/E430G/S440Y as described in Diebolder et al. Science (2014) 343(6176): 1260-3.

In some embodiments the Fc region comprises modification corresponding to the combination of substitutions M252Y/S254T/T256E as described in Dall'Acqua et al. J Immunol. (2002) 169:5171-5180.

In some embodiments the Fc region comprises modification corresponding to the combination of substitutions M428L/N434S as described in Zalevsky et al. Nat Biotechnol. (2010) 28:157-159.

In some embodiments the Fc region comprises modification corresponding to the combination of substitutions S267E/L328F as described in Chu et al., Mol Immunol. (2008) 45:3926-3933. In some embodiments the Fc region comprises modification corresponding to the combination of substitutions N325S/L328F as described in Shang et al. Biol Chem. (2014) 289:15309-15318.

In some embodiments the Fc region comprises modification to reduce/prevent an Fc-mediated function. In some embodiments the Fc region comprises modification to reduce/prevent ADCC. In some embodiments the Fc region comprises modification to reduce/prevent ADCP. In some embodiments the Fc region comprises modification to reduce/prevent CDC. An antigen-binding molecule comprising an Fc region comprising modification to reduce/prevent an Fc-mediated function (e.g. ADCC, ADCP, CDC) induces an reduced level of the relevant effector function as compared to an antigen-binding molecule comprising the corresponding unmodified Fc region.

In some embodiments the Fc region comprises modification to reduce/prevent binding to an Fc receptor. In some embodiments the Fc region comprises modification to reduce/prevent binding to an Fcγ receptor. In some embodiments the Fc region comprises modification to reduce/prevent binding to one or more of FcγRI, FcγRIIa, FcγRIIb, FcγRIIc, FcγRIIIa and FcγRIIIb. In some embodiments the Fc region comprises modification to reduce/prevent binding to FcγRIIIa. In some embodiments the Fc region comprises modification to reduce/prevent binding to FcγRIIa. In some embodiments the Fc region comprises modification to reduce/prevent binding to FcγRIIb. In some embodiments the Fc region comprises modification to reduce/prevent binding to a complement protein. In some embodiments the Fc region comprises modification to reduce/prevent binding to C1q. In some embodiments the Fc region comprises modification to reduce/prevent glycosylation of the amino acid residue corresponding to N297.

In some embodiments the Fc region is not able to induce one or more Fc-mediated functions (i.e. lacks the ability to elicit the relevant Fc-mediated function(s)). Accordingly, antigen-binding molecules comprising such Fc regions also lack the ability to induce the relevant function(s). Such antigen-binding molecules may be described as being devoid of the relevant function(s).

In some embodiments the Fc region is not able to induce ADCC. In some embodiments the Fc region is not able to induce ADCP. In some embodiments the Fc region is not able to induce CDC. In some embodiments the Fc region is not able to induce ADCC and/or is not able to induce ADCP and/or is not able to induce CDC.

In some embodiments the Fc region is not able to bind to an Fc receptor. In some embodiments the Fc region is not able to bind to an Fcγ receptor. In some embodiments the Fc region is not able to bind to one or more of FcγRI, FcγRIIa, FcγRIIb, FcγRIIc, FcγRIIIa and FcγRIIIb. In some embodiments the Fc region is not able to bind to FcγRIIIa. In some embodiments the Fc region is not able to bind to FcγRIIa. In some embodiments the Fc region is not able to bind to FcγRIIb. In some embodiments the Fc region is not able to bind to FcRn. In some embodiments the Fc region is not able to bind to a complement protein. In some embodiments the Fc region is not able to bind to C1q. In some embodiments the Fc region is not glycosylated at the amino acid residue corresponding to N297.

In some embodiments the Fc region comprises modification corresponding to N297A or N297Q or N297G as described in Leabman et al., MAbs. (2013) 5:896-903. In some embodiments the Fc region comprises modification corresponding to L235E as described in Alegre et al., J Immunol. (1992) 148:3461-3468. In some embodiments the Fc region comprises modification corresponding to the combination of substitutions L234A/L235A or F234A/L235A as described in Xu et al., Cell Immunol. (2000) 200:16-26. In some embodiments the Fc region comprises modification corresponding to P329A or P329G as described in Schlothauer et al., Protein Engineering, Design and Selection (2016), 29(10): 457-466. In some embodiments the Fc region comprises modification corresponding to the combination of substitutions L234A/L235A/P329G as described in Lo et al. J. Biol. Chem (2017) 292(9): 3900-3908. In some embodiments the Fc region comprises modification corresponding to the combination of substitutions described in Rother et al., Nat Biotechnol. (2007) 25:1256-1264. In some embodiments the Fc region comprises modification corresponding to the combination of substitutions S228P/L235E as described in Newman et al., Clin. Immunol. (2001) 98:164-174. In some embodiments the Fc region comprises modification corresponding to the combination of substitutions H268Q/V309L/A330S/P331S as described in An et al., MAbs. (2009) 1:572-579. In some embodiments the Fc region comprises modification corresponding to the combination of substitutions V234A/G237A/P238S/H268A/V309L/A330S/P331S as described in Vafa et al., Methods. (2014) 65:114-126. In some embodiments the Fc region comprises modification corresponding to the combination of substitutions L234A/L235E/G237A/A330S/P331S as described in US 2015/0044231 A1.

The combination of substitutions “L234A/L235A” and corresponding substitutions (such as e.g. F234A/L235A in human IgG4) are known to disrupt binding of Fc to Fcγ receptors and inhibit ADCC, ADCP, and also to reduce C1q binding and thus CDC (Schlothauer et al., Protein Engineering, Design and Selection (2016), 29(10): 457-466, hereby incorporated by reference in entirety). The substitutions “P329G” and “P329A” reduce C1q binding (and thereby CDC). Substitution of “N297” with “A”, “G” or “Q” is known to eliminate glycosylation, and thereby reduce Fc binding to C1q and Fcγ receptors, and thus CDC and ADCC. Lo et al. J. Biol. Chem (2017) 292(9): 3900-3908 (hereby incorporated by reference in its entirety) reports that the combination of substitutions L234A/L235A/P329G eliminated complement binding and fixation as well as Fcγ receptor dependent, antibody-dependent, cell-mediated cytotoxicity in both murine IgG2a and human IgG1.

The combination of substitutions L234A/L235E/G237A/A330S/P331S in IgG1 Fc is disclosed in US 2015/0044231 A1 to abolish induction of phagocytosis, ADCC and CDC.

In some embodiments the Fc region comprises modification corresponding to the substitution S228P as described in Silva et al., J Biol Chem. (2015) 290(9): 5462-5469. The substitution S228P in IgG4 Fc reduces Fab-arm exchange (Fab arm exchange can be undesirable).

In some embodiments the Fc region comprises modification corresponding to corresponding to the combination of substitutions L234A/L235A. In some embodiments the Fc region comprises modification corresponding to corresponding to the substitution P329G. In some embodiments the Fc region comprises modification corresponding to corresponding to the substitution N297Q.

In some embodiments the Fc region comprises modification corresponding to corresponding to the combination of substitutions L234A/L235A/P329G.

In some embodiments the Fc region comprises modification corresponding to corresponding to the combination of substitutions L234A/L235A/P329G/N297Q.

In some embodiments the Fc region comprises modification corresponding to corresponding to the combination of substitutions L234A/L235E/G237A/A330S/P331S.

In some embodiments the Fc region comprises modification corresponding to corresponding to the substitution S228P, e.g. in IgG4.

In some embodiments, the antigen-binding molecule of the present invention comprises an Fc region comprising modification in one or more of the CH2 and CH3 regions promoting association of the Fc region. Recombinant co-expression of constituent polypeptides of an antigen-binding molecule and subsequent association leads to several possible combinations. To improve the yield of the desired combinations of polypeptides in antigen-binding molecules in recombinant production, it is advantageous to introduce in the Fc regions modification(s) promoting association of the desired combination of heavy chain polypeptides. Modifications may promote e.g. hydrophobic and/or electrostatic interaction between CH2 and/or CH3 regions of different polypeptide chains. Suitable modifications are described e.g. in Ha et al., Front. Immnol (2016) 7:394, which is hereby incorporated by reference in its entirety.

In some embodiments the antigen antigen-binding molecule of the present invention comprises an Fc region comprising paired substitutions in the CH3 regions of the Fc region according to one of the following formats, as shown in Table 1 of Ha et al., Front. Immnol (2016) 7:394: KiH, KiHₛ₋ₛ, HA-TF, ZW1, 7.8.60, DD-KK, EW-RVT, EW-RVTₛ₋ₛ, SEED or A107.

In some embodiments, the Fc region comprises the “knob-into-hole” or “KiH” modification, e.g. as described e.g. in U.S. Pat. No. 7,695,936 and Carter, J Immunol Meth 248, 7-15 (2001). In such embodiments, one of the CH3 regions of the Fc region comprises a “knob” modification, and the other CH3 region comprises a “hole” modification. The “knob” and “hole” modifications are positioned within the respective CH3 regions so that the “knob” can be positioned in the “hole” in order to promote heterodimerisation (and inhibit homodimerisation) of the polypeptides and/or stabilise heterodimers. Knobs are constructed by substituting amino acids having small chains with those having larger side chains (e.g. tyrosine or tryptophan). Holes are created by substituting amino acids having large side chains with those having smaller side chains (e.g. alanine or threonine).

In some embodiments, one of the CH3 regions of the Fc region of the antigen-binding molecule of the present invention comprises the substitution (numbering of positions/substitutions in the Fc, CH2 and CH3 regions herein is according to the EU numbering system as described in Kabat et al., Sequences of Proteins of Immunological Interest, 5th Ed. Public Health Service, National Institutes of Health, Bethesda, MD, 1991) T366W, and the other CH3 region of the Fc region comprises the substitution Y407V. In some embodiments, one of the CH3 regions of the Fc region of the antigen-binding molecule comprises the substitution T366W, and the other CH3 region of the Fc region comprises the substitutions T366S and L368A. In some embodiments, one of the CH3 regions of the Fc region of the antigen-binding molecule comprises the substitution T366W, and the other CH3 region of the Fc region comprises the substitutions Y407V, T366S and L368A.

In some embodiments, the Fc region comprises the “DD-KK” modification as described e.g. in WO 2014/131694 A1. In some embodiments, one of the CH3 regions comprises the substitutions K392D and K409D, and the other CH3 region of the Fc region comprises the substitutions E356K and D399K. The modifications promote electrostatic interaction between the CH3 regions.

In some embodiments, the antigen-binding molecule of the present invention comprises an Fc region modified as described in Labrijn et al., Proc Natl Acad Sci USA. (2013) 110(13): 5145-50, referred to as ‘Duobody’ format. In some embodiments one of the CH3 regions comprises the substitution K409R, and the other CH3 region of the Fc region comprises the substitution K405L.

In some embodiments, the antigen-binding molecule of the present invention comprises an Fc region comprising the “EEE-RRR” modification as described in Strop et al., J Mol Biol. (2012) 420(3): 204-19. In some embodiments one of the CH3 regions comprises the substitutions D221E, P228E and L368E, and the other CH3 region of the Fc region comprises the substitutions D221R, P228R and K409R.

In some embodiments, the antigen-binding molecule comprises an Fc region comprising the “EW-RVT” modification described in Choi et al., Mol Cancer Ther (2013) 12(12): 2748-59. In some embodiments one of the CH3 regions comprises the substitutions K360E and K409W, and the other CH3 region of the Fc region comprises the substitutions Q347R, D399V and F405T.

In some embodiments, one of the CH3 regions comprises the substitution S354C, and the other CH3 region of the Fc region comprises the substitution Y349C. Introduction of these cysteine residues results in formation of a disulphide bridge between the two CH3 regions of the Fc region, further stabilizing the heterodimer (Carter (2001), J Immunol Methods 248, 7-15).

In some embodiments, the Fc region comprises the “KiHₛ₋ₛ” modification. In some embodiments one of the CH3 regions comprises the substitutions T366W and S354C, and the other CH3 region of the Fc region comprises the substitutions T366S, L368A, Y407V and Y349C.

In some embodiments, the antigen-binding molecule of the present invention comprises an Fc region comprising the “SEED” modification as described in Davis et al., Protein Eng Des Sel (2010) 23(4): 195-202, in which β-strand segments of human IgG1 CH3 and IgA CH3 are exchanged.

In some embodiments, one of the CH3 regions comprises the substitutions S364H and F405A, and the other CH3 region of the Fc region comprises the substitutions Y349T and T394F (see e.g. Moore et al., MAbs (2011) 3(6): 546-57).

In some embodiments, one of the CH3 regions comprises the substitutions T350V, L351Y, F405A and Y407V, and the other CH3 region of the Fc region comprises the substitutions T350V, T366L, K392L and T394W (see e.g. Von Kreudenstein et al., MAbs (2013) 5(5): 646-54).

In some embodiments, one of the CH3 regions comprises the substitutions K360D, D399M and Y407A, and the other CH3 region of the Fc region comprises the substitutions E345R, Q347R, T366V and K409V (see e.g. Leaver-Fay et al., Structure (2016) 24(4): 641-51).

In some embodiments, one of the CH3 regions comprises the substitutions K370E and K409W, and the other CH3 region of the Fc region comprises the substitutions E357N, D399V and F405T (see e.g. Choi et al., PLOS One (2015) 10(12): e0145349).

In some embodiments, the antigen-binding molecule of the present invention comprises an Fc region which does not bind to an Fc γ receptor. In some embodiments, the antigen-binding molecule comprises an Fc region which does not bind to one or more of FcγRI, FcγRIIa, FcγRIIb, FcγRIIc, FcγRIIIa and FcγRIIIb. In some embodiments, the antigen-binding molecule comprises an Fc region which does not bind to one or more of FcγRIIa, FcγRIIb and FcγRIIIa. In some embodiments, the antigen-binding molecule comprises an Fc region which does not bind to one or both of FcγRIIa and FcγRIIb.

The ability of an Fc region, or an antigen-binding molecule comprising an Fc region, to bind to a reference protein (e.g. an Fc receptor) can be analysed according to methods well known in the art, such as ELISA, immunoblot, immunoprecipitation, Surface Plasmon Resonance (SPR; see e.g. Hearty et al., Methods Mol Biol (2012) 907:411-442) or Bio-Layer Interferometry (BLI; see e.g. Lad et al., (2015) J Biomol Screen 20(4): 498-507).

As used herein, an Fc region “which does not bind to” a reference protein may display substantially no binding to the reference protein, e.g. as determined by ELISA, immunoblot (e.g. western blot), immunoprecipitation, SPR or BLI). “Substantially no binding” may be a level of interaction that is not significantly greater than the level of interaction determined for proteins that do not bind to one another in a given assay. “Substantially no binding” may be a level of interaction which is ≤5 times, e.g. ≤4 times, ≤3 times, ≤2.5 times, ≤2 times or ≤1.5 times the level of interaction determined for proteins that do not bind to one another, in a given assay.

In some embodiments, the antigen-binding molecule comprises an Fc region which binds to FcRn.

In some embodiments, the antigen-binding molecule comprises an Fc region which binds to FcRn, and which does not bind to one or more of FcγRIIa, FcγRIIb and FcγRIIIa. In some embodiments, the antigen-binding molecule comprises an Fc region which binds to FcRn, and which does not bind to one or both of FcγRIIa and FcγRIIb.

In some embodiments, the antigen-binding molecule of the present invention comprises an Fc region which does not induce ADCC. In some embodiments, the antigen-binding molecule of the present invention comprises an Fc region which does not induce ADCP. In some embodiments, the antigen-binding molecule of the present invention comprises an Fc region which does not induce CDC. In some embodiments, the antigen-binding molecule of the present invention comprises an Fc region which does not induce ADCC, ADCP or CDC.

As used herein, an Fc region/antigen-binding molecule which does not induce (i.e. is not able to induce) ADCC/ADCP/CDC elicits substantially no ADCC/ADCP/CDC activity, e.g. as determined by analysis in an appropriate assay for the relevant activity. “Substantially no ADCC/ADCP/CDC activity” refers to a level of ADCC/ADCP/CDC that is not significantly greater than ADCC/ADCP/CDC determined for an appropriate negative control molecule in a given assay (e.g. an antigen-binding molecule lacking an Fc region, or an antigen-binding molecule comprising a ‘silent’ Fc region (e.g. as described in Schlothauer et al., Protein Engineering, Design and Selection (2016), 29(10): 457-466, which is incorporated by reference hereinabove)). “Substantially no activity” may be a level of the relevant activity which is ≤5 times, e.g. ≤4 times, ≤3 times, ≤2.5 times, ≤2 times or ≤1.5 times the level of activity determined for an appropriate negative control molecule in a given assay.

The ability of an Fc region, or an antigen-binding molecule comprising an Fc region, to induce ADCC can be analysed e.g. according to the method described in Yamashita et al., Scientific Reports (2016) 6:19772 (hereby incorporated by reference in its entirety), or by 51Cr release assay as described e.g. in Jedema et al., Blood (2004) 103:2677-82 (hereby incorporated by reference in its entirety). The ability of an Fc region, or an antigen-binding molecule comprising an Fc region, to induce ADCP can be analysed e.g. according to the method described in Kamen et al., J Immunol (2017) 198 (1 Supplement) 157.17 (hereby incorporated by reference in its entirety). The ability of an Fc region, or an antigen-binding molecule comprising an Fc region, to induce CDC can be analysed e.g. using a C1q binding assay, e.g. as described in Schlothauer et al., Protein Engineering, Design and Selection (2016), 29(10): 457-466 (incorporated by reference hereinabove).

In some embodiments, the antigen-binding molecule comprises an Fc region comprising a polypeptide having an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:254. In some embodiments, the antigen-binding molecule comprises an Fc region comprising a polypeptide having an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:257. In some embodiments, the antigen-binding molecule comprises an Fc region comprising a polypeptide having an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:259. In some embodiments, the antigen-binding molecule comprises an Fc region comprising a polypeptide having an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:260.

In some embodiments the antigen-binding molecules of the present invention lack an Fc region.

### Fc Receptors

Fc receptors are polypeptides which bind to the Fc region of immunoglobulins. Fc receptor structure and function is reviewed e.g. in Masuda et al., Inflamm Allergy Drug Targets (2009) 8(1): 80-86, and Bruhns, Blood (2012) 119:5640-5649, both of which are hereby incorporated by reference in their entirety.

Fc receptors are expressed at surface of hematopoietic cells including macrophages, neutrophils, dendritic cells, eosinophils, basophils, mast cells, and NK cells. They include the IgG-binding Fc γ receptors, the high-affinity receptor for IgE (FcεRI), the IgA receptor, and the polymeric Ig receptor for IgA and IgM. The neonatal Fc receptor (FcRn) is a further Fc receptor for IgG, and is involved in IgG transport across epithelial barriers (transcytosis), protecting IgG from degradation, and antigen presentation. Humans have six different classes of Fcγ receptor (mouse orthologues are shown in brackets): FcγRI (mFcγRI), FcγRIIa (mFcγRIII), FcγRIIb (mFcγRIIb), FcγRIIc, FcγRIIIa (mFcγRIV) and FcγRIIIb.

FcγRI, FcγRIIa, FcγRIIc and FcγRIIIa comprise immunoreceptor tyrosine-based activation motifs (ITAMs) in their intracellular domains, and ligation by Fc leads to activation of cells expressing the receptors. FcγRIIb comprises immunoreceptor tyrosine-based inhibitory motifs (ITIMs) in its intracellular domain, and negatively regulates cell activation and degranulation, cell proliferation, endocytosis, and phagocytosis upon ligation by Fc.

In this specification an “Fcγ receptor” may be from any species, and includes isoforms, fragments, variants (including mutants) or homologues from any species. Similarly, “FcγRI”, “FcγRIIa”, “FcγRIIb”, “FcγRIIc”, “FcγRIIIa” and “FcγRIIIb” refer respectively to FcγRI/FcγRIIa/FcγRIIb/FcγRIIc/FcγRIIIa/FcγRIIIb from any species, and include isoforms, fragments, variants (including mutants) or homologues from any species.

In some embodiments, the Fcγ receptor (e.g. FcγRI/FcγRIIa/FcγRIIb/FcγRIIc/FcγRIIIa/FcγRIIIb) is from a mammal (e.g. a primate (rhesus, cynomolgous, non-human primate or human) and/or a rodent (e.g. rat or mouse). Isoforms, fragments, variants or homologues may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature isoform of an Fc γ receptor (e.g. FcγRI/FcγRIIa/FcγRIIb/FcγRIIc/FcγRIIIa/FcγRIIIb) from a given species, e.g. human.

Isoforms, fragments, variants or homologues may optionally be functional isoforms, fragments, variants or homologues, e.g. having a functional property/activity of the reference Fc γ receptor, as determined by analysis by a suitable assay for the functional property/activity. For example, an isoform, fragment, variant or homologue of FcγRI may e.g. display association with human IgG1 Fc.

In this specification an “FcRn receptor” may be from any species, and includes isoforms, fragments, variants (including mutants) or homologues from any species.

In some embodiments, the FcRn receptor is from a mammal (e.g. a primate (rhesus, cynomolgous, non-human primate or human) and/or a rodent (e.g. rat or mouse). Isoforms, fragments, variants or homologues may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature isoform of an FcRn receptor from a given species, e.g. human.

Isoforms, fragments, variants or homologues may optionally be functional isoforms, fragments, variants or homologues, e.g. having a functional property/activity of the reference FcRn, as determined by analysis by a suitable assay for the functional property/activity. For example, an isoform, fragment, variant or homologue of FcRn may e.g. display association with human IgG1 Fc.

### Polypeptides

The present invention also provides polypeptide constituents of antigen-binding molecules. The polypeptides may be provided in isolated or substantially purified form.

The antigen-binding molecule of the present invention may be, or may comprise, a complex of polypeptides.

In the present specification where a polypeptide comprises more than one domain or region, it will be appreciated that the plural domains/regions are preferably present in the same polypeptide chain. That is, the polypeptide comprises more than one domain or region is a fusion polypeptide comprising the domains/regions.

In some embodiments a polypeptide according to the present invention comprises, or consists of, a VH as described herein. In some embodiments a polypeptide according to the present invention comprises, or consists of, a VL as described herein.

In some embodiments, the polypeptide additionally comprises one or more antibody heavy chain constant regions (CH). In some embodiments, the polypeptide additionally comprises one or more antibody light chain constant regions (CL). In some embodiments, the polypeptide comprises a CH1, CH2 region and/or a CH3 region of an immunoglobulin (Ig).

In some embodiments the polypeptide comprises one or more regions of an immunoglobulin heavy chain constant sequence. In some embodiments the polypeptide comprises a CH1 region as described herein. In some embodiments the polypeptide comprises a CH1-CH2 hinge region as described herein. In some embodiments the polypeptide comprises a CH2 region as described herein. In some embodiments the polypeptide comprises a CH3 region as described herein.

In some embodiments the polypeptide comprises a CH2 and/or CH3 region comprising any one of the following amino acid substitutions/combinations of amino acid substitutions: F243L/R292P/Y300L/V305l/P396L; S239D/1332E; S239D/1332E/A330L; S298A/E333A/K334A; L234Y/L235Q/G236W/S239M/H268D/D270E/S298A; D270E/K326D/A330M/K334E; G236A/S239D/1332E; K326W/E333S; S267E/H268F/S324T; E345R/E430G/S440Y; M252Y/S254T/T256E; M428L/N434S; S267E/L328F; N325S/L328F; N297A; N297Q; N297G; L235E; L234A/L235A; F234A/L235A; P329A; P329G; L234A/L235A/P329G; H268Q/V309L/A330S/P331S; and V234A/G237A/P238S/H268A/V309L/A330S/P331S.

In some embodiments the polypeptide comprises a CH3 region comprising any one of the following amino acid substitutions/combinations of amino acid substitutions (shown e.g. in Table 1 of Ha et al., Front. Immnol (2016) 7:394, incorporated by reference hereinabove): T366W; T366S, L368A and Y407V; T366W and S354C; T366S, L368A, Y407V and Y349C; S364H and F405A; Y349T and T394F; T350V, L351Y, F405A and Y407V; T350V, T366L, K392L and T394W; K360D, D399M and Y407A; E345R, Q347R, T366V and K409V; K409D and K392D; D399K and E356K; K360E and K409W; Q347R, D399V and F405T; K360E, K409W and Y349C; Q347R, D399V, F405T and S354C; K370E and K409W; and E357N, D399V and F405T.

In some embodiments the CH2 and/or CH3 regions of the polypeptide comprise one or more amino acid substitutions for promoting association of the polypeptide with another polypeptide comprising a CH2 and/or CH3 region.

In some embodiments the polypeptide comprises one or more regions of an immunoglobulin light chain constant sequence. In some embodiments the polypeptide comprises a CL region as described herein.

In some embodiments the polypeptide lacks one or more regions of an immunoglobulin heavy chain constant sequence. In some embodiments the polypeptide lacks a CH2 region. In some embodiments the polypeptide lacks a CH3 region. In some embodiments the polypeptide lacks a CH2 region and also lacks a CH3 region.

In some embodiments, the polypeptide according to the present invention comprises a structure from N- to C-terminus according to one of the following:
(i) VH
(ii) VL
(iii) VH-CH1
(iv) VL-CL
(v) VL-CH1
(vi) VH-CL
(vii) VH-CH1-CH2-CH3
(viii) VL-CL-CH2-CH3
(ix) VL-CH1-CH2-CH3
(x) VH-CL-CH2-CH3

Also provided by the present invention are antigen-binding molecules composed of the polypeptides of the present invention. In some embodiments, the antigen-binding molecule of the present invention comprises one of the following combinations of polypeptides:
(A) VH+VL
(B) VH-CH1+VL-CL
(C) VL-CH1+VH-CL
(D) VH-CH1-CH2-CH3+VL-CL
(E) VH-CL-CH2-CH3+VL-CH1
(F) VL-CH1-CH2-CH3+VH-CL
(G) VL-CL-CH2-CH3+VH-CH1
(H) VH-CH1-CH2-CH3+VL-CL-CH2-CH3
(I) VH-CL-CH2-CH3+VL-CH1-CH2-CH3

In some embodiments the antigen-binding molecule comprises more than one of a polypeptide of the combinations shown in (A) to (I) above. By way of example, with reference to (D) above, in some embodiments the antigen-binding molecule comprises two polypeptides comprising the structure VH-CH1-CH2-CH3, and two polypeptides comprising the structure VL-CL.

In some embodiments, the antigen-binding molecule of the present invention comprises one of the following combinations of polypeptides:
(J) VH (anti-VISTA)+VL (anti-VISTA)
(K) VH (anti-VISTA)-CH1+VL (anti-VISTA)-CL
(L) VL (anti-VISTA)-CH1+VH (anti-VISTA)-CL
(M) VH (anti-VISTA)-CH1-CH2-CH3+VL (anti-VISTA)-CL
(N) VH (anti-VISTA)-CL-CH2-CH3+VL (anti-VISTA)-CH1
(O) VL (anti-VISTA)-CH1-CH2-CH3+VH (anti-VISTA)-CL
(P) VL (anti-VISTA)-CL-CH2-CH3+VH (anti-VISTA)-CH1
(Q) VH (anti-VISTA)-CH1-CH2-CH3+VL (anti-VISTA)-CL-CH2-CH3
(R) VH (anti-VISTA)-CL-CH2-CH3+VL (anti-VISTA)-CH1-CH2-CH3

Wherein: “VH (anti-VISTA)” refers to the VH of an antigen-binding molecule capable of binding to VISTA as described herein, e.g. as defined in one of (1) to (76); “VL (anti-VISTA)” refers to the VL of an antigen-binding molecule capable of binding to VISTA as described herein, e.g. as defined in one of (77) to (173).

In some embodiments the polypeptide comprises or consists of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of one of SEQ ID NOs: 212 to 243, 248 to 250, 258, 266 or 311 to 321.

### Linkers and Additional Sequences

In some embodiments the antigen-binding molecules and polypeptides of the present invention comprise a hinge region. In some embodiments a hinge region is provided between a CH1 region and a CH2 region. In some embodiments a hinge region is provided between a CL region and a CH2 region. In some embodiments the hinge region comprises, or consists of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:207.

In some embodiments the antigen-binding molecules and polypeptides of the present invention comprise one or more linker sequences between amino acid sequences. A linker sequence may be provided at one or both ends of one or more of a VH, VL, CH1-CH2 hinge region, CH2 region and a CH3 region of the antigen-binding molecule/polypeptide.

Linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, the linker sequence comprises at least one glycine residue and/or at least one serine residue. In some embodiments the linker sequence consists of glycine and serine residues. In some embodiments, the linker sequence has a length of 1-2, 1-3, 1-4, 1-5 or 1-10 amino acids.

The antigen-binding molecules and polypeptides of the present invention may additionally comprise further amino acids or sequences of amino acids. For example, the antigen-binding molecules and polypeptides may comprise amino acid sequence(s) to facilitate expression, folding, trafficking, processing, purification or detection of the antigen-binding molecule/polypeptide. For example, the antigen-binding molecule/polypeptide may comprise a sequence encoding a His, (e.g. 6×His), Myc, GST, MBP, FLAG, HA, E, or Biotin tag, optionally at the N- or C-terminus of the antigen-binding molecule/polypeptide. In some embodiments the antigen-binding molecule/polypeptide comprises a detectable moiety, e.g. a fluorescent, lunminescent, immuno-detectable, radio, chemical, nucleic acid or enzymatic label.

The antigen-binding molecules and polypeptides of the present invention may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides.

The signal peptide may be present at the N-terminus of the antigen-binding molecule/polypeptide, and may be present in the newly synthesised antigen-binding molecule/polypeptide. The signal peptide provides for efficient trafficking and secretion of the antigen-binding molecule/polypeptide. Signal peptides are often removed by cleavage, and thus are not comprised in the mature antigen-binding molecule/polypeptide secreted from the cell expressing the antigen-binding molecule/polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176).

### Labels and Conjugates

In some embodiments the antigen-binding molecules of the present invention additionally comprise a detectable moiety.

In some embodiments the antigen-binding molecule comprises a detectable moiety, e.g. a fluorescent label, phosphorescent label, luminescent label, immuno-detectable label (e.g. an epitope tag), radiolabel, chemical, nucleic acid or enzymatic label. The antigen-binding molecule may be covalently or non-covalently labelled with the detectable moiety.

Fluorescent labels include e.g. fluorescein, rhodamine, allophycocyanin, eosine and NDB, green fluorescent protein (GFP) chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Iodine¹²³, Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rhenium¹⁰⁵, Scandium⁴⁷, Tellurium^{121m}, Tellurium^{122m}, Tellurium^{125m}, Thulium¹⁶⁵, Thuliuml¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers. Enzymatic labels include e.g. peroxidase, alkaline phosphatase, glucose oxidase, beta-galactosidase and luciferase.

In some embodiments the antigen-binding molecules of the present invention are conjugated to a chemical moiety. The chemical moiety may be a moiety for providing a therapeutic effect. Antibody-drug conjugates are reviewed e.g. in Parslow et al., Biomedicines. 2016 September; 4(3): 14. In some embodiments, the chemical moiety may be a drug moiety (e.g. a cytotoxic agent). In some embodiments, the drug moiety may be a chemotherapeutic agent. In some embodiments, the drug moiety is selected from calicheamicin, DM1, DM4, monomethylauristatin E (MMAE), monomethylauristatin F (MMAF), SN-38, doxorubicin, duocarmycin, D6.5 and PBD.

### Particular Exemplary Embodiments of the Antigen-Binding Molecules

In some embodiments, the antigen-binding molecule comprises, or consists of:
(i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:331; and
(ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:317.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:212; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:213.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:214; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:215.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:216; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:217.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:218; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:219.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:220; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:221.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:222; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:223.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:224; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:225.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:226; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:227.

In some embodiments the antigen-binding molecule comprises, or consists of:
preferably one of 75%, 80%, 85%, 90%, 91%, 92%, 93%, 94%, 95%, 96%, 97%, 98%, 99% or 100% amino acid sequence identity to the amino acid sequence of SEQ ID NO:228; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:229.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:230; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:231.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:232; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:233.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:234; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:235.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:236; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:237.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:238; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:239.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:240; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:241.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:242; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:243.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:248; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:250.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:249; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:250.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:258; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:250.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:266; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:250.

In some embodiments the antigen-binding molecule comprises, or consists of:
(i) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:330; and
(ii) two polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:213.

In some embodiments, the antigen-binding molecule is produced by a cell of the cell line deposited 7 May 2021 as ATCC Patent Deposit Number PTA-127063, e.g. as described in GB 2108446.2, which is hereby incorporated by reference in its entirety.

### Functional Properties of the Antigen-Binding Molecules

The antigen-binding molecules described herein may be characterised by reference to certain functional properties. In some embodiments, the antigen-binding molecule described herein may possess one or more of the following properties:
binds to VISTA (e.g. human, murine and/or cynomolgus macaque VISTA);
does not bind to PD-L1 and/or HER3;
does not bind to an Fcγ receptor;
does not bind to C1q;
does not induce ADCC;
does not induce ADCP;
does not induce CDC;
binds to an FcRn receptor;
binds to VISTA-expressing cells;
inhibits interaction between VISTA and a binding partner for VISTA (e.g. PSGL-1, VSIG-3, VSIG-8 or LRIG1);
inhibits VISTA-mediated signalling;
inhibits VISTA-mediated signalling independently of Fc-mediated function;
increases killing of VISTA-expressing cells;
does not induce/increase killing of VISTA-expressing cells;
reduces the number/proportion of VISTA-expressing cells;
does not reduce the number/proportion of VISTA-expressing cells;
increases effector immune cell number/activity;
reduces suppressor immune cell number/activity;
reduces suppressor immune cell proliferation;
decreases immune suppression mediated by VISTA-expressing cells;
increases antigen presentation by antigen-presenting cells;
increases production of IL-6 by immune cells;
increases production of IFN-γ, IL-2 and/or IL-17 in a mixed lymphocyte reaction (MLR) assay;
increases T cell proliferation, IFN-γ production and/or TNFa production; and
inhibits the development and/or progression of cancer in vivo, and
does not induce cytokine release syndrome in vivo.

It will be appreciated that a given antigen-binding molecule may display more than one of the properties recited in the preceding paragraph. A given antigen-binding molecule may be evaluated for the properties recited in the preceding paragraph using suitable assays. The assays may be e.g. in vitro assays, which may be cell-free or cell-based assays. Alternatively, the assays may be e.g. in vivo assays, i.e. performed in non-human animals.

Where assays are cell-based assays, they may comprise contacting cells with a given antigen-binding molecule in order to determine whether the antigen-binding molecule displays one or more of the recited properties. Assays may employ species labelled with detectable entities in order to facilitate their detection. Assays may comprise evaluating the recited properties following treatment of cells separately with a range of quantities/concentrations of antigen-binding molecule (e.g. a dilution series). It will be appreciated that the cells are preferably cells that express VISTA, e.g. MDSCs.

Analysis of the results of such assays may comprise determining the concentration at which 50% of the maximal level of the relevant activity is attained. The concentration of antigen-binding molecule at which 50% of the maximal level of the relevant activity is attained may be referred to as the ‘half-maximal effective concentration’ of the antigen-binding molecule in relation to the relevant activity, which may also be referred to as the ‘EC₅₀’. By way of illustration, the EC₅₀ of a given antigen-binding molecule for binding to VISTA may be the concentration at which 50% of the maximal level of binding to the relevant species is achieved.

Depending on the property, the EC₅₀ may also be referred to as the ‘half-maximal inhibitory concentration’ or ‘IC₅₀’, this being the concentration of antigen-binding molecule at which 50% of the maximal level of inhibition of a given property is observed. By way of illustration, the IC₅₀ of a given antigen-binding molecule for inhibiting interaction between VISTA and an interaction partner for VISTA (e.g. LRIG1, PSGL-1, VSIG3 or VSIG8) may be the concentration at which 50% of the maximal level of inhibition is achieved.

The antigen-binding molecules described herein preferably display specific binding to VISTA. As used herein, “specific binding” refers to binding which is selective for the antigen, and which can be discriminated from non-specific binding to non-target antigen. An antigen-binding molecule that specifically binds to a target molecule preferably binds the target with greater affinity, and/or with greater duration than it binds to other, non-target molecules.

The ability of a given polypeptide to bind specifically to a given molecule can be determined by analysis according to methods known in the art, such as by ELISA, Surface Plasmon Resonance (SPR; see e.g. Hearty et al., Methods Mol Biol (2012) 907:411-442), Bio-Layer Interferometry (see e.g. Lad et al., (2015) J Biomol Screen 20(4): 498-507), flow cytometry, or by a radiolabeled antigen-binding assay (RIA) enzyme-linked immunosorbent assay. Through such analysis binding to a given molecule can be measured and quantified. In some embodiments, the binding may be the response detected in a given assay.

In some embodiments, the extent of binding of the antigen-binding molecule to an non-target molecule is less than about 10% of the binding of the antibody to the target molecule as measured, e.g. by ELISA, SPR, Bio-Layer Interferometry or by RIA. Alternatively, binding specificity may be reflected in terms of binding affinity where the antigen-binding molecule binds with a dissociation constant (K_{D}) that is at least 0.1 order of magnitude (i.e. 0.1×10ⁿ, where n is an integer representing the order of magnitude) greater than the K_{D} of the antigen-binding molecule towards a non-target molecule. This may optionally be one of at least 0.2, 0.3, 0.4, 0.5, 0.6, 0.7, 0.8, 0.9, 1.0, 1.5, or 2.0.

In some embodiments, the antigen-binding molecule displays binding to human VISTA, murine (e.g. mouse) VISTA and/or cynomolgus macaque (Macaca fascicularis) VISTA. That is, in some embodiments the antigen-binding molecule is cross-reactive for human VISTA and murine VISTA and/or cynomolgus macaque VISTA. In some embodiments the antigen-binding molecule of the present invention displays cross-reactivity with VISTA of a non-human primate. Cross-reactivity to VISTA in model species allows in vivo exploration of efficacy in syngeneic models without relying on surrogate molecules.

In some embodiments, the antigen-binding molecule does not display specific binding to PD-L1 (e.g. human PD-L1). In some embodiments, the antigen-binding molecule does not display specific binding to HER3 (e.g. human HER3). In some embodiments, the antigen-binding molecule does not display specific binding to (i.e. does not cross-react with) another member of the B7 family of proteins. In some embodiments, the antigen-binding molecule does not display specific binding to PD-L1, PD-L2 CD80, CD86, ICOSLG, CD276, VTCN1, NCR3LG1, HHLA2 and/or CTLA4.

In some embodiments, the antigen-binding molecule does not display specific binding to PD-1, PD-L1, B7H3, VTCN1 (B7H4), NCR3LG1 (B7H6), HHLA2 (B7H7) and/or CTLA4.

In some embodiments the antigen-binding molecule is not able to induce one or more Fc-mediated functions (i.e. lacks the ability to elicit the relevant Fc-mediated function(s)). Such antigen-binding molecules may be described as being devoid of the relevant function(s).

As explained hereinabove, an Fc region/antigen-binding molecule which does not induce (i.e. is not able to induce) ADCC/ADCP/CDC elicits substantially no ADCC/ADCP/CDC activity, e.g. as determined by analysis in an appropriate assay for the relevant activity. Similarly, an antigen-binding molecule “which does not bind to” a reference protein (e.g. a given Fc receptor or complement protein) may display substantially no binding to the reference protein in an appropriate assay.

In some embodiments the antigen-binding molecule is not able to induce ADCC. In some embodiments the antigen-binding molecule is not able to induce ADCP. In some embodiments the antigen-binding molecule is not able to induce CDC. In some embodiments the antigen-binding molecule is not able to induce ADCC and/or is not able to induce ADCP and/or is not able to induce CDC.

In some embodiments the antigen-binding molecule is not able to bind to an Fc receptor. In some embodiments the antigen-binding molecule is not able to bind to an Fcγ receptor. In some embodiments the antigen-binding molecule is not able to bind to one or more of FcγRI, FcγRIIa, FcγRIIb, FcγRIIc, FcγRIIIa and FcγRIIIb. In some embodiments the antigen-binding molecule is not able to bind to FcγRIIIa. In some embodiments the antigen-binding molecule is not able to bind to FcγRIIa. In some embodiments the antigen-binding molecule is not able to bind to FcγRIIb. In some embodiments the antigen-binding molecule binds to FcRn. In some embodiments the antigen-binding molecule is not able to bind to a complement protein. In some embodiments the antigen-binding molecule is not able to bind to C1q. In some embodiments the antigen-binding molecule is not glycosylated at the amino acid residue corresponding to N297.

In some embodiments the antigen-binding molecule binds to human VISTA, murine VISTA and/or cynomolgus macaque VISTA; and does not bind to PD-L1, PD-1, B7H3, VTCN1 (B7H4), NCR3LG1 (B7H6), HHLA2 (B7H7) and/or CTLA4 (e.g. human PD-L1/PD-1/B7H3/VTCN1/NCR3LG1/HHLA2/CTLA4).

In some embodiments, the antigen-binding molecule does not display specific binding to PD-L1 (e.g. human PD-L1). In some embodiments, the antigen-binding molecule does not display specific binding to HER3 (e.g. human HER3). In some embodiments, the antigen-binding molecule does not display specific binding to (i.e. does not cross-react with) another member of the B7 family of proteins. In some embodiments, the antigen-binding molecule does not display specific binding to PD-L1, PD-L2 CD80, CD86, ICOSLG, CD276, VTCN1, NCR3LG1, HHLA2 and/or CTLA4.

In some embodiments, the antigen-binding molecule does not display specific binding to PD-1, PD-L1, B7H3, VTCN1 (B7H4), NCR3LG1 (B7H6), HHLA2 (B7H7) and/or CTLA4.

In some embodiments, an antigen-binding molecule according to the present disclosure binds to VISTA with an affinity in the micromolar range, i.e. K_{D}=9.9×10⁻⁴ to 1×10⁻⁶ M. In some embodiments, an antigen-binding molecule binds to VISTA with sub-micromolar affinity, i.e. K_{D}<1×10⁻⁶ M. In some embodiments, an antigen-binding molecule binds to VISTA with an affinity in the nanomolar range, i.e. K_{D}=9.9×10⁻⁷ to 1×10⁻⁹ M. In some embodiments, an antigen-binding molecule binds to VISTA with sub-nanomolar affinity, i.e. K_{D}<1×10⁻⁹ M. In some embodiments, an antigen-binding molecule binds to VISTA with an affinity in the picomolar range, i.e. K_{D}=9.9×10⁻¹⁰ to 1×10⁻¹² M. In some embodiments, an antigen-binding molecule binds to VISTA with sub-picomolar affinity, i.e. K_{D}<1×10⁻¹² M. In some embodiments, the antigen-binding molecule described herein binds to VISTA (e.g. human VISTA, mouse VISTA) with a K_{D} of 10 μM or less, preferably one of ≤5 μM, ≤2 μM, ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM ≤3 nM, ≤2 nM, ≤1 nM or ≤500 pM. In some embodiments, the antigen-binding molecule binds to VISTA (e.g. human VISTA, mouse VISTA) with an affinity of K_{D}=≤10 nM, ≤9 nM, ≤8 nM, ≤7 nM or ≤6 nM, ≤5 nM, ≤4 nM, ≤3 nM, ≤2 nM or ≤1 nM. In some embodiments, the antigen-binding molecule binds to VISTA (e.g. human VISTA, mouse VISTA) with an affinity of K_{D}=$500 pM, ≤100 pM, ≤90 pM, ≤80 pM, ≤70 pM or ≤60 pM, ≤50 pM, ≤40 pM, ≤30 pM, ≤20 pM, ≤10 pM, ≤9 pM, ≤8 pM, ≤7 pM or ≤6 pM, ≤5 pM, ≤4 pM, ≤3 pM, ≤2 pM or ≤1 pM. In some embodiments, an antigen-binding molecule according to the present disclosure binds to VISTA with a K_{D} (e.g. as determined by SPR (Biocore) analysis, e.g. SPR analysis as described in the Examples of the present disclosure) of ≤1 nM (e.g. one of ≤900 pM, ≤800 pM, ≤700 pM, ≤600 pM, ≤500 pM, ≤400 pM, ≤300 pM).

In some embodiments, an antigen-binding molecule according to the present disclosure binds to human VISTA with a K_{D} (e.g. as determined by SPR (Biocore) analysis, e.g. SPR analysis as described in the Examples of the present disclosure) of ≤1 nM (e.g. one of ≤900 pM, ≤800 pM, ≤700 pM, ≤600 pM, ≤500 pM). In some embodiments, an antigen-binding molecule according to the present disclosure binds to cynomolgus macaque VISTA with a K_{D} (e.g. as determined by SPR (Biocore) analysis, e.g. SPR analysis as described in the Examples of the present disclosure) of ≤1 nM (e.g. one of ≤900 pM, ≤800 pM, ≤700 pM, ≤600 pM, ≤500 pM, ≤400 pM). In some embodiments, an antigen-binding molecule according to the present disclosure binds to rat VISTA with a K_{D} (e.g. as determined by SPR (Biocore) analysis, e.g. SPR analysis as described in the Examples of the present disclosure) of ≤1 nM (e.g. one of ≤900 pM, ≤800 pM, ≤700 pM, ≤600 pM, ≤500 pM, ≤400 pM). In some embodiments, an antigen-binding molecule according to the present disclosure binds to mouse VISTA with a K_{D} (e.g. as determined by SPR (Biocore) analysis, e.g. SPR analysis as described in the Examples of the present disclosure) of ≤1 nM (e.g. one of ≤900 pM, ≤800 pM, ≤700 pM, ≤600 pM).

In some embodiments, an antigen-binding molecule according to the present disclosure binds to VISTA with an EC₅₀ (e.g. as determined by ELISA, e.g. an ELISA as described in the Examples of the present disclosure) of 1 μM or less, e.g. one of ≤500 nM, ≤100 nM, ≤50 nM, ≤40 nM, ≤30 nM, ≤20 nM, ≤10 nM, ≤5 nM, ≤4 nM ≤3 nM, ≤2 nM, ≤1 nM, ≤500 pM, ≤400 pM, ≤300 pM, ≤200 pM, ≤100 pM, ≤50 pM, ≤40 pM, ≤30 pM, ≤20 pM, ≤15 pM, ≤10 pM, ≤5 pM or ≤1 pM.

In some embodiments, the antigen-binding molecule displays binding to human VISTA, murine (e.g. mouse) VISTA, rat VISTA, and/or cynomolgus macaque (Macaca fascicularis) VISTA. In some embodiments, the antigen-binding molecule binds to human VISTA and mouse VISTA and rat VISTA and cynomolgus macaque VISTA. In some embodiments, the antigen-binding molecule is cross-reactive for human VISTA and mouse VISTA and rat VISTA and cynomolgus macaque VISTA. In some embodiments, the antigen-binding molecule of the present disclosure displays cross-reactivity with VISTA of a non-human primate. Cross-reactivity to VISTA in model species allows in vivo exploration of efficacy in syngeneic models without relying on surrogate molecules.

In some embodiments, an antigen-binding molecule according to the present disclosure binds to human VISTA with an EC₅₀ (e.g. as determined by ELISA, e.g. an ELISA as described in the Examples of the present disclosure) of ≤20 pM (e.g. one of ≤15 pM, ≤12.5 pM, ≤10 pM, ≤7.5 pM). In some embodiments, an antigen-binding molecule according to the present disclosure binds to cynomolgus macaque VISTA with an EC₅₀ (e.g. as determined by ELISA, e.g. an ELISA as described in the Examples of the present disclosure) of ≤50 pM (e.g. one of ≤25 pM, ≤20 pM, ≤15 pM). In some embodiments, an antigen-binding molecule according to the present disclosure binds to rat VISTA with an EC₅₀ (e.g. as determined by ELISA, e.g. an ELISA as described in the Examples of the present disclosure) of ≤20 pM (e.g. one of ≤15 pM, ≤12.5 pM, ≤10 pM, ≤7.5 pM). In some embodiments, an antigen-binding molecule according to the present disclosure binds to mouse VISTA with an EC₅₀ (e.g. as determined by ELISA, e.g. an ELISA as described in the Examples of the present disclosure) of ≤20 pM (e.g. one of ≤15 pM, ≤12.5 pM, ≤10 pM, ≤7.5 pM, ≤5 pM).

In some embodiments, an antigen-binding molecule according to the present disclosure binds to VISTA (e.g. human VISTA) with similar affinity at pH from 5.5 to pH 7.5. For example, in some embodiments, the antigen-binding molecule displays similar affinity for VISTA at pH 5.5 as the affinity for VISTA at pH 7.5.

Herein, a binding affinity which is ‘similar’ to a reference binding affinity means a binding affinity which is within 50%, e.g. within one of 40%, 45%, 30%, 25%, 20% 19%, 18%, 17%, 16%, 15%, 14%, 13%, 12%, 11%, 10%, 9%, 8%, 7%, 6%, 5%, 4%, 3%, 2% or 1% of the reference binding affinity, as determined under comparable conditions.

The K_{D} for binding to VISTA (e.g. human VISTA) may be similar at pH from 5.5 to pH 7.5. The EC₅₀ for binding to VISTA (e.g. human VISTA) may be similar at pH from 5.5 to pH 7.5.

Herein, a ‘similar’ K_{D} or EC₅₀ value to a reference value may be ≥0.5 times and ≤2 times, e.g. one of ≥ 0.7 times and ≤1.5 times, ≥0.75 times and ≤1.25 times, ≥0.8 times and ≤1.2 times, ≥0.85 times and ≤ 1.15 times, ≥0.9 times and ≤1.1 times, ≥0.91 times and ≤1.09 times, ≥0.92 times and ≤1.08 times, ≥ 0.93 times and ≤1.07 times, ≥0.94 times and ≤1.06 times, ≥0.95 times and ≤1.05 times, ≥0.96 times and ≤1.04 times, ≥0.97 times and ≤1.03 times, ≥0.98 times and ≤1.02 times, or ≥0.99 times and ≤ 1.01 times the reference value.

The antigen-binding molecules of the present invention may bind to a particular region of interest of VISTA. The antigen-binding region of an antigen-binding molecule according to the present domain may bind to a linear epitope of VISTA, consisting of a contiguous sequence of amino acids (i.e. an amino acid primary sequence). In some embodiments, the antigen-binding region molecule may bind to a conformational epitope of VISTA, consisting of a discontinuous sequence of amino acids of the amino acid sequence.

In some embodiments, the antigen-binding molecule of the present invention is capable of binding to VISTA. In some embodiments, the antigen-binding molecule is capable of binding to VISTA in an extracellular region of VISTA. In some embodiments, the antigen-binding molecule is capable of binding to VISTA in the Ig-like V-type domain (e.g. the region shown in SEQ ID NO:6). In some embodiments, the antigen-binding molecule is capable of binding to VISTA in the region shown in SEQ ID NO:31.

In some embodiments the antigen-binding molecule is capable of binding to a polypeptide comprising or consisting of the amino acid sequence shown in SEQ ID NO:6. In some embodiments the antigen-binding molecule is capable of binding to a polypeptide comprising or consisting of the amino acid sequence shown in SEQ ID NO:31. In some embodiments the antigen-binding molecule is capable of binding to a peptide or polypeptide comprising or consisting of the amino acid sequence shown in SEQ ID NO:322. In some embodiments the antigen-binding molecule is capable of binding to a peptide or polypeptide comprising or consisting of the amino acid sequence shown in SEQ ID NO:26. In some embodiments the antigen-binding molecule is capable of binding to a peptide or polypeptide comprising or consisting of the amino acid sequence shown in SEQ ID NO:27. In some embodiments the antigen-binding molecule is capable of binding to a peptide or polypeptide comprising or consisting of the amino acid sequence shown in SEQ ID NO:28. In some embodiments the antigen-binding molecule is capable of binding to a peptide or polypeptide comprising or consisting of the amino acid sequence shown in SEQ ID NO:29. In some embodiments the antigen-binding molecule is capable of binding to a peptide or polypeptide comprising or consisting of the amino acid sequence shown in SEQ ID NO:30.

In some embodiments, the antigen-binding molecule does not bind to the region of VISTA bound by IGN175A (described e.g. in WO 2014/197849 A2). In some embodiments, the antigen-binding molecule does not bind to the region of VISTA bound by an antigen-binding molecule comprised of a polypeptide consisting of the sequence of SEQ ID NO:267 and a polypeptide consisting of the sequence of SEQ ID NO: 268.

In some embodiments, the antigen-binding molecule does not compete with IGN175A (described e.g. in WO 2014/197849 A2) for binding to VISTA. In some embodiments, the antigen-binding molecule does not compete with an antigen-binding molecule comprised of a polypeptide consisting of the sequence of SEQ ID NO: 267 and a polypeptide consisting of the sequence of SEQ ID NO:268 for binding to VISTA.

The ability of a given antigen-binding molecule to compete with IGN175A or the antigen-binding molecule comprised of a polypeptide consisting of the sequence of SEQ ID NO:267 and a polypeptide consisting of the sequence of SEQ ID NO:268 for binding to VISTA can be analysed e.g. by competition ELISA, or by epitope binning as described in Abdiche et al., J Immunol Methods (2012) 382 (—2): 101-116 (hereby incorporated by reference in its entirety). Epitope binning can be performed e.g. by BLI analysis, e.g. as described in Example 8 of the present application.

In some embodiments the antigen-binding molecule is not capable of binding to a peptide consisting of the amino acid sequence shown in SEQ ID NO:275.

As used herein, a “peptide” refers to a chain of two or more amino acid monomers linked by peptide bonds. A peptide typically has a length in the region of about 2 to 50 amino acids. A “polypeptide” is a polymer chain of two or more peptides. Polypeptides typically have a length greater than about 50 amino acids.

The ability of an antigen-binding molecule to bind to a given peptide/polypeptide can be analysed by methods well known to the skilled person, including analysis by ELISA, immunoblot (e.g. western blot), immunoprecipitation, surface plasmon resonance and biolayer interferometry.

In some embodiments the antigen-binding molecule is capable of binding the same region of VISTA, or an overlapping region of VISTA, to the region of VISTA which is bound by an antibody comprising the VH and VL sequences of one of clones 4M2-C12, 4M2-B4, 4M2-C9, 4M2-D9, 4M2-D5, 4M2-A8, V4H1, V4H2, V4-C1, V4-C9, V4-C24, V4-C26, V4-C27, V4-C28, V4-C30, V4-C31, 2M1-B12, 2M1-D2, 1M2- D2, 13D5p, 13D5-1, 13D5-13, 5M1-A11 or 9M2-C12.

In some embodiments the antigen-binding molecule is capable of binding to a region of VISTA which is different to the region of VISTA bound by IGN175A (described e.g. in WO 2014/197849 A2). In some embodiments the antigen-binding molecule is capable of binding to a region of VISTA which is different to the region of VISTA bound by an antigen-binding molecule comprised of a polypeptide consisting of the sequence of SEQ ID NO:267 and a polypeptide consisting of the sequence of SEQ ID NO:268.

In some embodiments the antigen-binding molecule is capable of binding to a region of VISTA which does not overlap the region of VISTA bound by IGN175A (described e.g. in WO 2014/197849 A2). In some embodiments the antigen-binding molecule is capable of binding to a region of VISTA which does not overlap with the region of VISTA bound by an antigen-binding molecule comprised of a polypeptide consisting of the sequence of SEQ ID NO:267 and a polypeptide consisting of the sequence of SEQ ID NO: 268.

In some embodiments, the antigen-binding molecule binds to VISTA through contact with residues of VISTA which are non-identical to the residues of VISTA which are contacted by VSTB112 (described e.g. in WO 2015/097536 A2). In some embodiments, the antigen-binding molecule binds to VISTA through contact with residues of VISTA which are non-identical to the residues of VISTA which are contacted by an antigen-binding molecule comprised of a polypeptide consisting of the sequence of SEQ ID NO:269 and a polypeptide consisting of the sequence of SEQ ID NO:270.

In some embodiments the epitope for the antigen-binding molecule is non-identical to the epitope for VSTB112. In some embodiments the epitope for the antigen-binding molecule is non-identical to the epitope for an antigen-binding molecule comprised of a polypeptide consisting of the sequence of SEQ ID NO: 269 and a polypeptide consisting of the sequence of SEQ ID NO:270.

The region of a peptide/polypeptide to which an antibody binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3): 145-156, which is hereby incorporated by reference in its entirety.

In some embodiments the antigen-binding molecule of the present invention binds to VISTA in a region which is accessible to an antigen-binding molecule (i.e., an extracellular antigen-binding molecule) when VISTA is expressed at the cell surface (i.e. in or at the cell membrane). In some embodiments the antigen-binding molecule is capable of binding to VISTA expressed at the cell surface of a cell expressing VISTA. In some embodiments the antigen-binding molecule is capable of binding to VISTA-expressing cells (e.g. CD14+ monocytes (such as monocyte-derived suppressor cells (MDSCs)) and/or CD33+ myeloid cells, tumor associated macrophages (TAMs), and neutrophils).

The ability of an antigen-binding molecule to bind to a given cell type can be analysed by contacting cells with the antigen-binding molecule, and detecting antigen-binding molecule bound to the cells, e.g. after a washing step to remove unbound antigen-binding molecule. The ability of an antigen-binding molecule to bind to immune cell surface molecule-expressing cells and/or cancer cell antigen-expressing cells can be analysed by methods such as flow cytometry and immunofluorescence microscopy.

The antigen-binding molecule of the present invention may be an antagonist of VISTA. In some embodiments, the antigen-binding molecule is capable of inhibiting a function or process (e.g. interaction, signalling or other activity) mediated by VISTA and/or a binding partner for VISTA (e.g. PSGL-1, VSIG-3, VSIG-8, LRIG1). Herein, ‘inhibition’ refers to a reduction, decrease or lessening relative to a control condition. An antigen-binding molecule which inhibits a given interaction/activity/process may be referred to as inhibitor or antagonist of the interaction/activity/process, and may be said to ‘block’ or ‘neutralise’ the interaction/activity/process.

VISTA-binding antigen-binding molecules described herein are able to inhibit VISTA-mediated functions/processes by a mechanism not requiring Fc-mediated functions such as ADCC, ADCP and CDC. That is, VISTA-binding antigen-binding molecules described herein are able to inhibit the immunosuppressive activity of VISTA-expressing cells without the need to elicit ADCC, ADCP and/or CDC.

In particular, VISTA-binding antigen-binding molecules described herein are able to inhibit VISTA via a mechanism not requiring binding to Fcγ receptors and/or binding to C1q.

In some embodiments the antigen-binding molecule of the present invention is capable of inhibiting interaction between VISTA and a binding/interaction partner for VISTA (e.g. PSGL-1, VSIG-3, VSIG-8, LRIG1). In some embodiments the antigen-binding molecule of the present invention is capable of inhibiting interaction between VISTA and PSGL-1. In some embodiments the antigen-binding molecule of the present invention is capable of inhibiting interaction between VISTA and VSIG-3. In some embodiments the antigen-binding molecule of the present invention is capable of inhibiting interaction between VISTA and LRIG1.

The ability of an antigen-binding molecule to inhibit interaction between two factors can be determined for example by analysis of interaction in the presence of, or following incubation of one or both of the interaction partners with, the antibody/fragment. Assays for determining whether a given antigen-binding molecule is capable of inhibiting interaction between two interaction partners include competition ELISA assays and analysis by SPR.

An antigen-binding molecule which is capable of inhibiting a given interaction (e.g. between VISTA and a binding partner for VISTA) is identified by the observation of a reduction/decrease in the level of interaction between the interaction partners in the presence of—or following incubation of one or both of the interaction partners with—the antigen-binding molecule, as compared to the level of interaction in the absence of the antigen-binding molecule (or in the presence of an appropriate control antigen-binding molecule). Suitable analysis can be performed in vitro, e.g. using recombinant interaction partners or using cells expressing the interaction partners. Cells expressing interaction partners may do so endogenously, or may do so from nucleic acid introduced into the cell. For the purposes of such assays, one or both of the interaction partners and/or the antigen-binding molecule may be labelled or used in conjunction with a detectable entity for the purposes of detecting and/or measuring the level of interaction.

The ability of an antigen-binding molecule to inhibit interaction between two binding partners can also be determined by analysis of the downstream functional consequences of such interaction. For example, downstream functional consequences of interaction between VISTA and a binding partner for VISTA may include VISTA-mediated signalling. For example, the ability of an antigen-binding molecule to inhibit interaction of VISTA and a binding partner for VISTA may be determined by analysis of production of IL-2, IFN-γ and/or IL-17 in an MLR assay.

In some embodiments, the antigen-binding molecule of the present invention is capable of inhibiting interaction between VISTA and a binding partner for VISTA (e.g. PSGL-1, VSIG-3, VSIG-8, LRIG1) to less than less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of interaction between VISTA and the binding partner for VISTA in the absence of the antigen-binding molecule (or in the presence of an appropriate control antigen-binding molecule).

In some embodiments the antigen-binding molecule inhibits VISTA-mediated signalling. In some embodiments, VISTA-mediated signalling may be signalling mediated by a polypeptide complex comprising VISTA. In some embodiments, VISTA-mediated signalling may be signalling mediated by a polypeptide complex comprising VISTA and an interaction partner for VISTA (e.g. LRIG1, VSIG3, PSGL-1, VSIG8). VISTA-mediated signalling can be analysed e.g. using an assay of effector immune cell number/activity, such as an MLR assay as described in the experimental examples herein. Inhibition of VISTA-mediated signalling can be identified by detection of an increase in the number and/or activity of effector immune cells, as determined e.g. by an increase in production of IL-2, IFN-γ and/or IL-17.

The ability of an antigen-binding molecule to inhibit interaction between VISTA and an interaction partner for VISTA can be determined for example by analysis of interaction in the presence of, or following incubation of one or both of the interaction partners with, the antigen-binding molecule. Assays for determining whether a given antigen-binding molecule is capable of inhibiting interaction between VISTA and an interaction partner for VISTA include competition ELISA assays and analysis by SPR.

An antigen-binding molecule which is capable of inhibiting interaction between VISTA and an interaction partner for VISTA may identified by the observation of a reduction/decrease in the level of interaction between the interaction partners in the presence of—or following incubation of one or both of the interaction partners with—the antigen-binding molecule, as compared to the level of interaction in the absence of the antigen-binding molecule (or in the presence of an appropriate control antigen-binding molecule known not to inhibit such interaction). Suitable analysis can be performed in vitro, e.g. using recombinant interaction partners or using cells expressing the interaction partners. Cells expressing interaction partners may do so endogenously, or may do so from nucleic acid introduced into the cell. For the purposes of such assays, one or both of the interaction partners and/or the antigen-binding molecule may be labelled or used in conjunction with a detectable entity for the purposes of detecting and/or measuring the level of interaction.

In some embodiments the antigen-binding molecule is able to inhibit VISTA-mediated signalling by a mechanism not requiring or involving Fc-mediated function. In some embodiments the antigen-binding molecule is able to inhibit VISTA-mediated signalling independently of Fc-mediated function. That is, in some embodiments the antigen-binding molecule is able to inhibit VISTA-mediated signalling in an Fc region-independent manner.

The ability of an antigen-binding molecule to inhibit VISTA-mediated signalling by a mechanism not requiring/involving Fc-mediated function can be evaluated e.g. by analysing the ability of the antigen-binding molecule provided in a format lacking a functional Fc region to inhibit VISTA-mediated signalling. For example, the effect on VISTA-mediated signalling can be investigated using an antigen-binding molecule comprising a ‘silent’ Fc region (e.g. comprising LALA PG substitutions), or using an antigen-binding molecule provided in a format lacking an Fc region (e.g. scFv, Fab etc.).

In some embodiments the antigen-binding molecule is able to inhibit VISTA-mediated signalling by a mechanism not involving ADCC. In some embodiments the antigen-binding molecule is able to inhibit VISTA-mediated signalling by a mechanism not involving ADCP. In some embodiments the antigen-binding molecule is able to inhibit VISTA-mediated signalling by a mechanism not involving CDC.

In some embodiments the antigen-binding molecule is able to inhibit VISTA-mediated signalling by a mechanism not requiring binding of the antigen-binding molecule to an Fc receptor. In some embodiments the antigen-binding molecule is able to inhibit VISTA-mediated signalling by a mechanism not requiring binding of the antigen-binding molecule to an Fcγ receptor. In some embodiments the antigen-binding molecule is able to inhibit VISTA-mediated signalling by a mechanism not requiring binding of the antigen-binding molecule to one or more of FcγRI, FcγRIIa, FcγRIIb, FcγRIIc, FcγRIIIa and FcγRIIIb. In some embodiments the antigen-binding molecule is able to inhibit VISTA-mediated signalling by a mechanism not requiring binding to FcγRIIIa. In some embodiments the antigen-binding molecule is able to inhibit VISTA-mediated signalling by a mechanism not requiring binding to FcγRIIa. In some embodiments the antigen-binding molecule is able to inhibit VISTA-mediated signalling by a mechanism not requiring binding to FcγRIIb. In some embodiments the antigen-binding molecule is able to inhibit VISTA-mediated signalling by a mechanism not requiring binding to a complement protein. In some embodiments the antigen-binding molecule is able to inhibit VISTA-mediated signalling by a mechanism not requiring binding to C1q. In some embodiments the antigen-binding molecule is able to inhibit VISTA-mediated signalling by a mechanism not requiring N297 glycosylation.

In some embodiments, the antigen-binding molecule of the present invention is capable of increasing killing of VISTA-expressing cells. Killing of VISTA-expressing cells may be increased through an effector function of the antigen-binding molecule. In embodiments wherein antigen-binding molecule comprises an Fc region the antigen-binding molecule may increase killing of VISTA-expressing cells through one or more of complement dependent cytotoxicity (CDC), antibody-dependent cell-mediated cytotoxicity (ADCC) and antibody-dependent cellular phagocytosis (ADCP).

An antigen-binding molecule which is capable of increasing killing of VISTA-expressing cells can be identified by observation of an increased level of killing of VISTA-expressing cells in the presence of—or following incubation of the VISTA-expressing cells with—the antigen-binding molecule, as compared to the level of cell killing detected in the absence of the antigen-binding molecule (or in the presence of an appropriate control antigen-binding molecule), in an appropriate assay. Assays of CDC, ADCC and ADCP are well known the skilled person. The level of killing of VISTA-expressing cells can also be determined by measuring the number/proportion of viable and/or non-viable VISTA-expressing cells following exposure to different treatment conditions.

In some embodiments, the antigen-binding molecule of the present invention is capable of increasing killing of VISTA-expressing cells (e.g. VISTA-expressing MDSCs) to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of killing observed in the absence of the antigen-binding molecule (or in the presence of an appropriate control antigen-binding molecule).

In some embodiments, the antigen-binding molecule of the present invention is capable of reducing the number of VISTA-expressing cells (e.g. VISTA-expressing MDSCs) to less than less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the number of VISTA-expressing cells (e.g. VISTA-expressing MDSCs, TAMs, neutrophils) detected following incubation in the absence of the antigen-binding molecule (or following incubation in the presence of an appropriate control antigen-binding molecule), in a comparable assay.

In some embodiments the antigen-binding molecule is a non-depleting antigen-binding molecule. That is, in some embodiments the antigen-binding molecule does not cause substantial depletion of VISTA-expressing cells. In some embodiments the antigen-binding molecule does not elicit/increase ADCC, ADCP and/or CDC against VISTA-expressing cells.

In some embodiments, the antigen-binding molecule of the present invention does not induce/increase killing of VISTA-expressing cells, e.g. in embodiments wherein the antigen-binding molecule lacks an Fc region, or embodiments wherein the antigen-binding molecule comprises an Fc region which is not able to induce an Fc-mediated antibody effector function. In some embodiments, the antigen-binding molecule of the present invention does not reduce the number/proportion of VISTA-expressing cells.

In some embodiments the antigen-binding molecule of the present invention (i) inhibits VISTA-mediated signalling, and (ii) does not induce/increase killing of VISTA-expressing cells. In some embodiments the antigen-binding molecule of the present invention (i) inhibits VISTA-mediated signalling, and (ii) does not reduce the number/proportion of VISTA-expressing cells.

This can be particularly advantageous, because VISTA is expressed by cells that it is not desirable to deplete. For example, VISTA is expressed at low levels by immune cells (e.g. certain types of T cells and dendritic cells) that it is not desirable to kill or reduce the number/proportion of.

In some embodiments, the antigen-binding molecule of the present invention is capable of increasing the number and/or activity of effector immune cells relative to a negative control condition, e.g. in an appropriate in vitro assay, or in vivo. By way of explanation, the antigen-binding molecules of the invention may be capable of releasing effector immune cells from MDSC-mediated suppression of effector immune cell proliferation and function. In some embodiments the effector immune cells may be e.g. CD8+ T cells, CD8+ cytotoxic T lymphocytes (CD8+ CTLs), CD4+ T cells, CD4+ T helper cells, NK cells, IFNγ-producing cells, memory T cells, central memory T cells, antigen-experienced T cells or CD45RO+ T cells.

Cell numbers and proportions can be determined e.g. by flow cytometry analysis using antibodies allowing detection of cell types. Cell division can be analysed, for example, by in vitro analysis of incorporation of 3H-thymidine or by CFSE dilution assay, e.g. as described in Fulcher and Wong, Immunol Cell Biol (1999) 77(6): 559-564, hereby incorporated by reference in entirety. Effector immune cell activity can be analysed by measuring a correlate of such activity. In some embodiments effector immune cell activity can be determined e.g. by analysis of production of IL-2, IFN-γ and/or IL-17.

In some embodiments, the antigen-binding molecule of the present invention is capable of increasing the number of an effector immune cell type to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the number observed in the absence of the antigen-binding molecule (or in the presence of an appropriate control antigen-binding molecule). In some embodiments, the antigen-binding molecule of the present invention is capable of increasing the level of a correlate of effector immune cell activity to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present invention is capable of decreasing the level of immune suppression mediated by VISTA-expressing cells. A change in the level of immune suppression may be determined using methods to measure the expression of arginase 1 and/or the production of reactive oxygen species (ROS) by VISTA-expressing cells, for example as described in Ochoa et al., Ann Surg. 2001 March; 233(3): 393-399 and Dikalov and Harrison Antioxid Redox Signal. 2014 Jan. 10; 20(2): 372-382.

In some embodiments, the antigen-binding molecule of the present invention is capable of increasing antigen presentation by antigen-presenting cells, e.g. as determined using a suitable assay of antigen presentation. In some embodiments, the antigen-binding molecule of the present invention is capable of increasing phagocytosis by phagocytic cells (e.g. neutrophils, monocytes, macrophages, mast cells, and/or dendritic cells), e.g. as determined using a suitable assay of the level of phagocytosis.

In some embodiments, the antigen-binding molecule of the present disclosure is capable of increasing the number and/or activity of antigen-presenting cells (e.g. CD11b+ MHCII+ cells) relative to a negative control condition, e.g. in an appropriate in vitro assay, or in vivo (e.g. in a tumor). In some embodiments, the antigen-binding molecule is capable of increasing the number and/or activity of macrophages (e.g. CD11b+ F4/80+ cells) relative to a negative control condition, e.g. in an appropriate in vitro assay, or in vivo (e.g. in a tumor). In some embodiments, the antigen-binding molecule is capable of increasing the number and/or activity of dendritic cells (e.g. CD11c+ cells) relative to a negative control condition, e.g. in an appropriate in vitro assay, or in vivo (e.g. in a tumor).

In some embodiments, the antigen-binding molecule of the present disclosure is capable of increasing the number of a cell type recited in the preceding paragraph to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the number observed in the absence of the antigen-binding molecule (or in the presence of an appropriate control antigen-binding molecule). In some embodiments, the antigen-binding molecule of the present disclosure is capable of increasing the level of a correlate of activity of a cell type recited in the preceding paragraph to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present invention is capable of increasing production of IL-6 by immune cells. The immune cells may be e.g. PBMCs, lymphocytes, T cells, B cells, NK cells, or monocytes. In some embodiments the immune cells are monocytes. In some embodiments the antigen-binding molecule is capable of increasing production of IL-6 by immune cells following stimulation, e.g. with LPS. The ability of an antigen-binding molecule to increase production of IL-6 by immune cells can be analysed in an in vitro assay e.g. as described in Example 10 herein. Such methods may comprise stimulating monocytes (e.g. THP1 cells) with LPS, and incubating the stimulated cells with the antigen-binding molecule.

In some embodiments, the antigen-binding molecule of the present invention is capable of increasing IL-6 production by immune cells (e.g. LPS-stimulated THP1 cells) to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present disclosure is capable of increasing the number and/or activity of Th1/Th17 cells. In some embodiments, the antigen-binding molecule is capable of upregulating the Th1/Th17 response. In some embodiments, the antigen-binding molecule favours the Th1/Th17 response over the Th2 response. In some embodiments, the antigen-binding molecule of the present disclosure is capable of increasing T cell proliferation, IL-2 production, IFN-γ production, TNFα production and/or IL-17A production in a Mixed Lymphocyte Reaction (MLR) assay. MLR assays may be performed as described in Bromelow et al J. Immunol Methods, 2001 Jan. 1; 247(1-2): 1-8, (hereby incorporated by reference in its entirety), or as described in the experimental examples herein. IL-2, IFNγ and/or IL-17 production may be analysed e.g. by antibody-based methods well known to the skilled person, such as western blot, immunohistochemistry, immunocytochemistry, flow cytometry, ELISA, ELISPOT, or by reporter-based methods.

In some embodiments, the antigen-binding molecule of the present invention is capable of increasing T cell (e.g. Th1/Th17 cell) proliferation, IL-2 production, IFN-γ production and/or IL-17 production in a Mixed Lymphocyte Reaction (MLR) assay. MLR assays may be performed as described in Bromelow et al J. Immunol Methods, 2001 Jan. 1; 247(1-2): 1-8, (hereby incorporated by reference in its entirety), or as described in the experimental examples herein. IL-2, IFNγ and/or IL-17 production may be analysed e.g. by antibody-based methods well known to the skilled person, such as western blot, immunohistochemistry, immunocytochemistry, flow cytometry, ELISA, ELISPOT, or by reporter-based methods.

In some embodiments, the antigen-binding molecule of the present invention is capable of increasing T cell proliferation, IL-2 production, IFN-γ production and/or IL-17 production in an MLR assay to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present invention is capable of increasing T cell proliferation, IFN-γ production and/or TNFa production, e.g. in the presence of VISTA/VISTA expressing cells. Antigen-binding molecules may be evaluated for such properties e.g. in in vitro assays as described in the experimental examples herein.

In some embodiments, the antigen-binding molecule of the present invention is capable of increasing T cell (e.g. Th1/Th17 cell) proliferation, IFN-γ production and/or TNFa production (e.g. in the presence of VISTA/VISTA expressing cells) to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present invention is capable of increasing T cell (e.g. CD4+ T cell and/or CD8+ T cell, e.g. Th1/Th17 cell) proliferation to a greater extent than a VISTA-binding antibody disclosed in the prior art (e.g. VSTB112, described e.g. in WO 2015/097536 A2). T cell proliferation may be evaluated in an in vitro assay e.g. as described in Example 9 herein, and may involve stimulating T cell proliferation by culture in the presence of agonist anti-CD3 antibody. In some embodiments, the antigen-binding molecule of the present invention is capable of increasing T cell proliferation in such an assay to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level proliferation induced by the prior art VISTA-binding antibody (e.g. VSTB112).

In some embodiments, the antigen-binding molecule of the present disclosure is capable of increasing T cell-mediated lysis of cancer cells to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present disclosure is capable of increasing T cell-mediated lysis of cancer cells, e.g. in the presence of VISTA/VISTA expressing cells. Antigen-binding molecules may be evaluated for such properties e.g. in in vitro assays as described in the experimental examples herein.

In some embodiments, the antigen-binding molecule of the present disclosure is capable of increasing T cell-mediated lysis (e.g. in the presence of VISTA/VISTA expressing cells) to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule (or in the presence of an appropriate control antigen-binding molecule).

In some embodiments, the antigen-binding molecule of the present invention is capable of increasing IL-6 production by THP1 cells to a greater extent than a VISTA-binding antibody disclosed in the prior art (e.g. VSTB112, described e.g. in WO 2015/097536 A2). IL-6 production by THP1 cells may be evaluated in an in vitro assay e.g. as described in Example 10 herein, and may involve stimulating THP1 cells with LPS. In some embodiments, the antigen-binding molecule of the present invention is capable of increasing IL-6 production in such an assay to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level induced by the prior art VISTA-binding antibody (e.g. VSTB112).

In some embodiments, the antigen-binding molecule of the present invention is capable of: reducing the number and/or activity of suppressor immune cells, inhibiting proliferation of suppressor immune cells, and/or reducing the proportion of suppressor immune cells within a population of cells (e.g. CD45+ cells, e.g. CD45+ cells obtained from a tumor) relative to control condition, e.g. as determined in an appropriate in vitro assay, or in vivo.

The suppressor immune cells may be e.g. VISTA-expressing cells, Arg1-expressing cells, MDSCs, granulocytic MDSCs (g-MDSCs) or monocytic MDSCs (m-MDSCs). In some embodiments, the suppressor immune cells are CD11b+ GR1+ MHCII-cells.

In some embodiments, the reduction in the number/activity/proliferation/proportion is to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the number/activity/proliferation/proportion observed in the absence of the antigen-binding molecule (or in the presence of an appropriate control antigen-binding molecule).

In some embodiments the antigen-binding molecule is able to reduce the number/activity/proliferation/proportion of suppressor immune cells by a mechanism not involving Fc-mediated function. In some embodiments the antigen-binding molecule is able to reduce the number/activity/proliferation/proportion of suppressor immune cells independently of Fc-mediated function (i.e. in an Fc region-independent manner). In some embodiments the antigen-binding molecule is able to reduce the number/activity/proliferation/proportion of suppressor immune cells by a mechanism not involving ADCC, ADCP and/or CDC. In some embodiments the antigen-binding molecule is able to reduce the number/activity/proliferation/proportion of suppressor immune cells by a mechanism not involving depletion of VISTA-expressing cells.

In some embodiments, the antigen-binding molecule of the present invention inhibits the development and/or progression of cancer in vivo.

In some embodiments the antigen-binding molecule causes an increase in the killing of cancer cells, e.g. by effector immune cells. In some embodiments the antigen-binding molecule causes a reduction in the number of cancer cells in vivo, e.g. as compared to an appropriate control condition. In some embodiments the antigen-binding molecule inhibits tumor growth, e.g. as determined by measuring tumor size/volume over time.

In some embodiments, the antigen-binding molecule of the present invention is capable of increasing serum levels of IFN-γ and/or IL-23 in mice treated with the antigen-binding molecule. Serum levels of IFN-γ and/or IL-23 can be analysed e.g. by ELISA of serum derived from blood samples obtained from the mice. In some embodiments, administration of the antigen-binding molecule of the present invention increases serum level of IFN-γ and/or IL-23 to more than 1 times, e.g. ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of administration of the antigen-binding molecule (or the level observed following administration of an appropriate control antigen-binding molecule).

The antigen-binding molecule of the present invention may be analysed for the ability to inhibit development and/or progression of cancer in an appropriate in vivo model, e.g. cell line-derived xenograft model such as CT26 cell-derived model, a 4T-1 cell-derived model, an LL2 cell-derived model, a B16 cell-derived model, or an EL4 cell-derived model. The cancer may be a cancer in which VISTA-expressing cells and/or MDSCs (e.g. VISTA-expressing MDSCs, TAMs, neutrophils) are pathologically implicated. Cancers in which MDSCs are ‘pathologically implicated’ include cancers in which MDSCs, or an increased number/proportion of MDSCs, is positively associated with onset, development or progression of the cancer, and/or severity of one or more symptoms of the cancer, or a cancer for which MDSCs, or an increased number/proportion of MDSCs, is a risk factor for the onset, development or progression of the cancer. The cancer may comprise MDSCs in an organ/tissue which is affected by the disease (e.g. an organ/tissue in which the symptoms of the disease/condition manifest) or in a tumor.

In some embodiments, administration of an antigen-binding molecule according to the present invention may cause one or more of: inhibition of the development/progression of the cancer, a delay to/prevention of onset of the cancer, a reduction in/delay to/prevention of tumor growth, a reduction in/delay to/prevention of metastasis, a reduction in the severity of the symptoms of the cancer, a reduction in the number of cancer cells, a reduction in tumour size/volume, and/or an increase in survival (e.g. progression free survival), e.g. as determined in an CT26 cell, 4T-1 cell, an LL2 cell, a B16 cell, or an EL4 cell-derived xenograft model.

In some embodiments, administration of the antigen-binding molecule of the present invention is capable of inhibiting greater than 5%, e.g. ≥10%, ≥15%, ≥20%, ≥25%, ≥30%, ≥35%, ≥40%, ≥45%, ≥50%, ≥55%, ≥60%, ≥65%, ≥70%, ≥75%, ≥80%, ≥85%, ≥90% or ≥95% of the tumor growth observed in the absence of administration of the antigen-binding molecule (or following administration of an appropriate control antigen-binding molecule).

In some embodiments, administration of the antigen-binding molecule at the dose and periodicity described in the experiments in the CT26 cell-derived model of the experimental examples of the present disclosure inhibits greater than 5%, e.g. ≥10%, ≥15%, ≥20%, ≥25%, ≥30%, ≥35%, ≥40%, ≥45%, ≥50%, ≥55%, ≥60%, ≥65%, ≥70%, ≥75% or ≥80% of the tumor growth observed in the absence of administration of the antigen-binding molecule (or following administration of an appropriate control antigen-binding molecule). In some embodiments, administration of the antigen-binding molecule at the dose and periodicity described in the experiments in the 4T-1 cell-derived model of the experimental examples of the present disclosure inhibits greater than 5%, e.g. ≥10%, ≥15%, ≥20%, ≥25%, ≥30%, ≥35%, ≥40%, ≥45%, ≥50% of the tumor growth observed in the absence of administration of the antigen-binding molecule (or following administration of an appropriate control antigen-binding molecule).

In some embodiments, administration of an antigen-binding molecule according to the present disclosure is not associated with cytokine release syndrome. In some embodiments, administration of an antigen-binding molecule is not associated with the systemic activation of leukocytes (e.g. B cells, T cells, NK cells, macrophages, dendritic cells and/or monocytes). In some embodiments, administration of an antigen-binding molecule is not associated with systemic upregulation of expression of inflammatory cytokines and/or chemokines (e.g. IL-6, IFN-γ, IL-8, IL-10, GM-CSF, MIP-1α/β, MCP-1, CXCL9 and/or CXCL10) by leukocytes.

In some embodiments, treatment of a subject with an antigen-binding molecule or other article disclosed herein (e.g. composition, nucleic acid etc), e.g. wherein the antigen-binding molecule/article is administered to a subject at a dosage and/or in accordance with a dosage schedule described herein, may be associated with one or more of the following outcomes:
Absence of adverse events (AEs);
Absence of serious adverse events (SAEs);
Minimal or reduced frequency of adverse events (AEs), e.g. as compared to other anti-VISTA therapeutic antibodies;
Minimal or reduced frequency of serious adverse events (SAEs), e.g. as compared to other anti-VISTA therapeutic antibodies;
Absence of dose limiting toxicities (DLTs);
Minimal or reduced frequency of dose limiting toxicities (DLTs), e.g. as compared to other anti-VISTA therapeutic antibodies, such as W0180 and CA-170;
Reduction of circulating tumour markers after treatment (e.g. cell-free (cf) DNA alteration allele fraction/tumour fraction, ctDNA), e.g. compared to the same subject before treatment;
Absence of, minimal, or reduced frequency of infusion related reactions, e.g. cytokine release syndrome and related cytokine increases, e.g. as compared to other anti-VISTA therapeutic antibodies, such as W0180 and CA-170;
Absence of, minimal, or reduced frequency of gastrointestinal toxicity, e.g. as compared to other anti-VISTA therapeutic antibodies, such as W0180 and CA-170;
Absence of, minimal, or reduced frequency of rash or dermatitis, e.g. as compared to other anti-VISTA therapeutic antibodies, such as W0180 and CA-170;
Absence of, minimal, or reduced frequency of haematological changes, e.g. as compared to other anti-VISTA therapeutic antibodies, such as W0180 and CA-170;
Absence of, minimal, or reduced frequency of cardiotoxicity, e.g. as compared to other anti-VISTA therapeutic antibodies, such as W0180 and CA-170; and/or.
Absence of, minimal, or reduced frequency of albumin increase, e.g. as compared to other anti-VISTA therapeutic antibodies, such as W0180 and CA-170.

An adverse event (AE) can be defined as any untoward, undesired or unplanned medical occurrence in a patient administered an investigational medicinal product (IMP), a comparator product or an approved drug. An AE can be a sign, symptom, disease, and/or laboratory or physiological observation that may or may not be related to the IMP or comparator. An AE includes but is not limited to those in the following list.
A clinically significant worsening of a pre-existing condition. This includes conditions that may resolve completely and then become abnormal again.
AEs occurring from an overdose of an IMP, whether accidental or intentional.
AEs occurring from lack of efficacy of an IMP, for example, if the Investigator suspects that a drug batch is not efficacious or if the Investigator suspects that the IMP has contributed to disease progression.

A serious adverse event (SAE) is any AE, regardless of dose, causality or expectedness, that:
results in death;
is life-threatening, i.e. an event when the patient was at substantial risk of dying at the time of the adverse event occurring or with continued use of the device or other medical product which might have resulted in the death of the patient;
requires in-patient hospitalisation or prolongs existing in-patient hospitalisation (some hospitalisations are exempt from SAE reporting e.g. hospital admissions planned prior to the patient entering the trial; overnight stays for planned procedures such a blood transfusions);
results in persistent or significant incapacity or disability;
is a congenital anomaly or birth defect;
is any other medically important event, i.e. any event that may jeopardise the patient or may require intervention to prevent one of the outcomes listed above.

In some embodiments, response to treatment in accordance with the present disclosure can be characterised by reference to tumour/lesion responses. In some embodiments, tumour/lesion responses are evaluated in accordance with the response evaluation criteria in solid tumours (RECIST) criteria, e.g. the RECIST 1.1 criteria as described in Eisenhauer et al., Eur J Cancer. 2009 January; 45(2): 228-47, which is hereby incorporated by reference in its entirety.

In some embodiments treatment of a subject with an antigen-binding molecule or article described herein, e.g. wherein the antigen-binding molecule/article is administered to a subject at a dosage and/or in accordance with a dosage schedule described herein, may be associated with one or more of the following outcomes (as assessed in accordance with the RECIST 1.1 criteria, as appropriate; see Example 19.9 for details and methods of assessment), e.g. at 12 and/or 24 months from the start of treatment:
An anti-tumour response;
A complete response (CR). A CR refers to a complete macroscopic disappearance of all target and/or non-target tumours. A CR may involve normalisation of tumour marker level;
Increased likelihood of a complete response (CR), e.g. as compared to the likelihood of CR in the same subject without treatment with the antigen-binding molecule, to a subject who has not received the antigen-binding molecule, or to a subject that has been treated with a different anti-VISTA antigen-binding molecule;
An increased proportion of subjects displaying a complete response (CR), e.g. as compared to the proportion of subjects displaying a CR who have not been treated with the antigen-binding molecule, or who have been treated with a different anti-VISTA antigen-binding molecule;
Overall survival (OS). OS is defined as the time from the start of treatment to death due to any cause;
Increased likelihood of overall survival (OS), e.g. as compared to the likelihood of OS in the same subject without treatment with the antigen-binding molecule, to a subject who has not received the antigen-binding molecule, or to a subject that has been treated with a different anti-VISTA antigen-binding molecule;
An increased proportion of subjects demonstrating overall survival (OS), e.g. as compared to the proportion of subjects displaying OS who have not been treated with the antigen-binding molecule, or who have been treated with a different anti-VISTA antigen-binding molecule;
Progression-free survival (PFS). PFS refers to the time from start of treatment to disease progression or death.
Increased likelihood of progression-free survival (PFS), e.g. as compared to the likelihood of PFS in the same subject without treatment with the antigen-binding molecule, to a subject who has not received the antigen-binding molecule, or to a subject that has been treated with a different anti-VISTA antigen-binding molecule;
An increased proportion of subjects demonstrating progression-free survival (PFS), e.g. as compared to the proportion of subjects displaying PFS who have not been treated with the antigen-binding molecule, or who have been treated with a different anti-VISTA antigen-binding molecule;
Progression-free survival at 6 months (PFS6). PFS6 refers to the percentage of patients alive and progression-free at 6 months (26 weeks) after the start of treatment.
Increased likelihood of progression-free survival at 6 months (PFS6), e.g. as compared to the likelihood of PFS in the same subject without treatment with the antigen-binding molecule, to a subject who has not received the antigen-binding molecule, or to a subject that has been treated with a different anti-VISTA antigen-binding molecule;
An increased percentage of subjects demonstrating progression-free survival at 6 months (PFS6), e.g. as compared to the percentage of subjects displaying PFS6 who have not been treated with the antigen-binding molecule, or who have been treated with a different anti-VISTA antigen-binding molecule;
A partial response (PR). PR refers to a reduction of at least 30% in the sum of all target tumour diameters compared to baseline sum diameters calculated before treatment;
Increased likelihood of a partial response (PR), e.g. as compared to the likelihood of a PR in the same subject without treatment with the antigen-binding molecule, to a subject who has not received the antigen-binding molecule, or to a subject that has been treated with a different anti-VISTA antigen-binding molecule;
An increased proportion of subjects demonstrating a partial response (PR), e.g. as compared to the proportion of subjects displaying a PR who have not been treated with the antigen-binding molecule, or who have been treated with a different anti-VISTA antigen-binding molecule;
A mixed response (MR). MR refers to one or more tumour lesions fulfilling the criteria for PR and other tumour lesion(s) fulfilling the criteria for progressive disease (at least a 20% increase in the sum of all tumour diameters from the smallest tumour size and/or the appearance of a new tumour lesion);
Increased likelihood of a mixed response (MR), e.g. as compared to the likelihood of a MR in the same subject without treatment with the antigen-binding molecule, to a subject who has not received the antigen-binding molecule, or to a subject that has been treated with a different anti-VISTA antigen-binding molecule;
An increased proportion of subjects demonstrating a mixed response (MR), e.g. as compared to the proportion of subjects displaying a MR who have not been treated with the antigen-binding molecule, or who have been treated with a different anti-VISTA antigen-binding molecule;
Stable disease (SD). SD refers to neither partial response nor progressive disease compared to the tumour burden at the start of treatment.
Increased likelihood of stable disease (SD), e.g. as compared to the likelihood of SD in the same subject without treatment with the antigen-binding molecule, to a subject who has not received the antigen-binding molecule, or to a subject that has been treated with a different anti-VISTA antigen-binding molecule;
An increased proportion of subjects demonstrating stable disease (SD), e.g. as compared to the proportion of subjects displaying SD who have not been treated with the antigen-binding molecule, or who have been treated with a different anti-VISTA antigen-binding molecule;
An increased duration of response (DoR) or increased duration of CR, e.g. as compared to the duration in the same subject without treatment with the antigen-binding molecule, to a subject who has not received the antigen-binding molecule, or to a subject that has been treated with a different anti-VISTA antigen-binding molecule. Duration of response (DoR) is defined as the time from the date measurement criteria are first met for PR or CR to the date measurement criteria are first met for progressive disease (PD). Duration of CR (DoCR) is defined as the time from the date when the measurement criteria are met for CR to the date measurement criteria are first met for PD;
An increased proportion of subjects demonstrating an increased duration of response (DoR) or increased duration of CR, e.g. as compared to the DoR or DoCR of subjects without treatment with the antigen-binding molecule, or who have been treated with a different anti-VISTA antigen-binding molecule;
An overall response (OR). OR is defined as achieving Complete Response (CR) or Partial Response (PR);
An overall response rate (ORR). ORR is defined as the proportion of patients who have achieved Complete Response (CR) or Partial Response (PR);
An increased ORR, e.g. as compared to the proportion of subjects displaying an OR who have not been treated with the antigen-binding molecule, or who have been treated with a different anti-VISTA antigen-binding molecule.

Tumour responses can be evaluated using the appropriate imaging technique according to the tumour and its location, e.g. CT scan, MRI scan and FDG-PET. Appropriate techniques will be familiar to the skilled person and are described in Eisenhauer et al, supra, and/or in Example 19.9 herein.

The subject may be a subject defined herein, e.g. having, or determined to have, a cancer or solid tumour, e.g. according to the present disclosure.

### Chimeric Antigen Receptors (CARs)

The present invention also provides Chimeric Antigen Receptors (CARs) comprising the antigen-binding molecules or polypeptides of the present invention.

CARs are recombinant receptors that provide both antigen-binding and T cell activating functions. CAR structure and engineering is reviewed, for example, in Dotti et al., Immunol Rev (2014) 257(1), hereby incorporated by reference in its entirety. CARs comprise an antigen-binding region linked to a cell membrane anchor region and a signalling region. An optional hinge region may provide separation between the antigen-binding region and cell membrane anchor region, and may act as a flexible linker.

The CAR of the present invention comprises an antigen-binding region which comprises or consists of the antigen-binding molecule of the present invention, or which comprises or consists of a polypeptide according to the invention.

The cell membrane anchor region is provided between the antigen-binding region and the signalling region of the CAR and provides for anchoring the CAR to the cell membrane of a cell expressing a CAR, with the antigen-binding region in the extracellular space, and signalling region inside the cell. In some embodiments, the CAR comprises a cell membrane anchor region comprising or consisting of an amino acid sequence which comprises, consists of, or is derived from, the transmembrane region amino acid sequence for one of CD3-ζ, CD4, CD8 or CD28. As used herein, a region which is ‘derived from’ a reference amino acid sequence comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the reference sequence.

The signalling region of a CAR allows for activation of the T cell. The CAR signalling regions may comprise the amino acid sequence of the intracellular domain of CD3-ζ, which provides immunoreceptor tyrosine-based activation motifs (ITAMs) for phosphorylation and activation of the CAR-expressing T cell. Signalling regions comprising sequences of other ITAM-containing proteins such as FcγRI have also been employed in CARs (Haynes et al., 2001 J Immunol 166(1): 182-187). Signalling regions of CARs may also comprise co-stimulatory sequences derived from the signalling region of co-stimulatory molecules, to facilitate activation of CAR-expressing T cells upon binding to the target protein. Suitable co-stimulatory molecules include CD28, OX40, 4-1BB, ICOS and CD27. In some cases CARs are engineered to provide for co-stimulation of different intracellular signalling pathways. For example, signalling associated with CD28 costimulation preferentially activates the phosphatidylinositol 3-kinase (P13K) pathway, whereas the 4-1BB-mediated signalling is through TNF receptor associated factor (TRAF) adaptor proteins. Signalling regions of CARs therefore sometimes contain co-stimulatory sequences derived from signalling regions of more than one co-stimulatory molecule. In some embodiments, the CAR of the present invention comprises one or more co-stimulatory sequences comprising or consisting of an amino acid sequence which comprises, consists of, or is derived from, the amino acid sequence of the intracellular domain of one or more of CD28, OX40, 4-1BB, ICOS and CD27.

An optional hinge region may provide separation between the antigen-binding domain and the transmembrane domain, and may act as a flexible linker. Hinge regions may be derived from IgG1. In some embodiments, the CAR of the present invention comprises a hinge region comprising or consisting of an amino acid sequence which comprises, consists of, or is derived from, the amino acid sequence of the hinge region of IgG1.

Also provided is a cell comprising a CAR according to the invention. The CAR according to the present invention may be used to generate CAR-expressing immune cells, e.g. CAR-T or CAR-NK cells. Engineering of CARs into immune cells may be performed during culture, in vitro.

The antigen-binding region of the CAR of the present invention may be provided with any suitable format, e.g. scFv, scFab, etc.

### Nucleic Acids and Vectors

The present invention provides a nucleic acid, or a plurality of nucleic acids, encoding an antigen-binding molecule, polypeptide or CAR according to the present invention.

In some embodiments, the nucleic acid is purified or isolated, e.g. from other nucleic acid, or naturally-occurring biological material. In some embodiments the nucleic acid(s) comprise or consist of DNA and/or RNA.

The present invention also provides a vector, or plurality of vectors, comprising the nucleic acid or plurality of nucleic acids according to the present invention.

The nucleotide sequence may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the invention.

The term “operably linked” may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. The resulting transcript(s) may then be translated into a desired peptide(s)/polypeptide(s).

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes).

In some embodiments, the vector may be a eukaryotic vector, e.g. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

Constituent polypeptides of an antigen-binding molecule according to the present invention may be encoded by different nucleic acids of the plurality of nucleic acids, or by different vectors of the plurality of vectors.

### Cells Comprising/Expressing the Antigen-Binding Molecules and Polypeptides

The present invention also provides a cell comprising or expressing an antigen-binding molecule, polypeptide or CAR according to the present invention. Also provided is a cell comprising or expressing a nucleic acid, a plurality of nucleic acids, a vector or a plurality of vectors according to the invention.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

The present invention also provides a method for producing a cell comprising a nucleic acid(s) or vector(s) according to the present invention, comprising introducing a nucleic acid, a plurality of nucleic acids, a vector or a plurality of vectors according to the present invention into a cell. In some embodiments, introducing an isolated nucleic acid(s) or vector(s) according to the invention into a cell comprises transformation, transfection, electroporation or transduction (e.g. retroviral transduction).

The present invention also provides a method for producing a cell expressing/comprising an antigen-binding molecule, polypeptide or CAR according to the present invention, comprising introducing a nucleic acid, a plurality of nucleic acids, a vector or a plurality of vectors according to the present invention in a cell. In some embodiments, the methods additionally comprise culturing the cell under conditions suitable for expression of the nucleic acid(s) or vector(s) by the cell. In some embodiments, the methods are performed in vitro.

The present invention also provides cells obtained or obtainable by the methods according to the present invention.

### Producing the Antigen-Binding Molecules and Polypeptides

Antigen-binding molecules and polypeptides according to the invention may be prepared according to methods for the production of polypeptides known to the skilled person.

Polypeptides may be prepared by chemical synthesis, e.g. liquid or solid phase synthesis. For example, peptides/polypeptides can by synthesised using the methods described in, for example, Chandrudu et al., Molecules (2013), 18:4373-4388, which is hereby incorporated by reference in its entirety.

Alternatively, antigen-binding molecules and polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008); 5(2): 135-146 both of which are hereby incorporated by reference in their entirety. Methods for the recombinant production of antigen-binding molecules are also described in Frenzel et al., Front Immunol. (2013); 4:217 and Kunert and Reinhart, Appl Microbiol Biotechnol. (2016) 100:3451-3461, both of which are hereby incorporated by reference in their entirety.

In some cases the antigen-binding molecule of the present invention are comprised of more than one polypeptide chain. In such cases, production of the antigen-binding molecules may comprise transcription and translation of more than one polypeptide, and subsequent association of the polypeptide chains to form the antigen-binding molecule.

For recombinant production according to the invention, any cell suitable for the expression of polypeptides may be used. The cell may be a prokaryote or eukaryote. In some embodiments the cell is a prokaryotic cell, such as a cell of archaea or bacteria. In some embodiments the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. CHO, HEK (e.g. HEK293), HeLa or COS cells. In some embodiments, the cell is a CHO cell that transiently or stably expresses the polypeptides.

In some cases the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. according using a system described in Zemella et al. Chembiochem (2015) 16(17): 2420-2431, which is hereby incorporated by reference in its entirety.

Production may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the antigen-binding molecule/polypeptide(s), the polypeptide(s) of interest may be isolated. Any suitable method for separating proteins from cells known in the art may be used. In order to isolate the polypeptide it may be necessary to separate the cells from nutrient medium. If the polypeptide(s) are secreted from the cells, the cells may be separated by centrifugation from the culture media that contains the secreted polypeptide(s) of interest. If the polypeptide(s) of interest collect within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation, or may be performed subsequently to precipitation.

Once the polypeptide(s) of interest have been isolated from culture it may be desired or necessary to concentrate the polypeptide(s). A number of methods for concentrating proteins are known in the art, such as ultrafiltration or lyophilisation.

### Compositions

The present invention also provides compositions comprising the antigen-binding molecules, polypeptides, CARs, nucleic acids, expression vectors and cells described herein.

The antigen-binding molecules, polypeptides, CARs, nucleic acids, expression vectors and cells described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion.

Suitable formulations may comprise the antigen-binding molecule in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments the composition is formulated for injection or infusion, e.g. into a blood vessel or tumor.

In accordance with the invention described herein methods are also provided for the production of pharmaceutically useful compositions, such methods of production may comprise one or more steps selected from: producing an antigen-binding molecule, polypeptide, CAR, nucleic acid (or plurality thereof), expression vector (or plurality thereof) or cell described herein; isolating an antigen-binding molecule, polypeptide, CAR, nucleic acid (or plurality thereof), expression vector (or plurality thereof) or cell described herein; and/or mixing an antigen-binding molecule, polypeptide, CAR, nucleic acid (or plurality thereof), expression vector (or plurality thereof) or cell described herein with a pharmaceutically acceptable carrier, adjuvant, excipient or diluent.

For example, a further aspect the invention described herein relates to a method of formulating or producing a medicament or pharmaceutical composition for use in the treatment of a disease/condition (e.g. a cancer), the method comprising formulating a pharmaceutical composition or medicament by mixing an antigen-binding molecule, polypeptide, CAR, nucleic acid (or plurality thereof), expression vector (or plurality thereof) or cell described herein with a pharmaceutically acceptable carrier, adjuvant, excipient or diluent.

In aspects and embodiments of the present disclosure, the antigen-binding molecule may be provided in a composition comprising particular chemical constituents in specified concentrations/proportions.

In some embodiments, the antigen-binding molecule is provided in a buffer. As used herein, a “buffer” refers to a buffered solution that resists changes in pH by the action of its acid-base conjugate components. A buffer of the present disclosure preferably has a pH in the range from about 4.5 to about 7.0, preferably from about 5.0 to about 6.5. Examples of buffers that will control the pH in this range include acetate, succinate, histidine, histidine-arginine, histidine-methionine and other organic acid buffers.

In some embodiments, the composition comprising the antigen-binding molecule has a pH of 4.0 to 7.0, e.g. one of pH 4.5 to pH 6.8, pH 4.6 to pH 6.5, pH 4.8 to pH 6.3, or pH 5.0 to pH 6.3. In some embodiments, the composition has a pH of ˜5.5. In some embodiments, the composition has a pH of ˜ 5.8. In some embodiments, the composition has a pH of ˜6.3.

In some embodiments, the antigen-binding molecule is provided in an acetate buffer, i.e. a buffer comprising acetate ions. In some embodiments, the antigen-binding molecule is provided in a composition comprising acetate at a final concentration of 2 mM to 200 mM acetate, e.g. one of 5 mM to 100 mM, 10 mM to 40 mM, 12 mM to 30 mM, 15 to 25 mM, or 18 to 22 mM. In some embodiments, the composition may comprise ˜20 mM acetate. The buffer may comprise sodium acetate.

In some embodiments, the antigen-binding molecule is provided in a histidine buffer, i.e. a buffer comprising histidine ions. In some embodiments, the antigen-binding molecule is provided in a composition comprising histidine at a final concentration of 2 mM to 200 mM histidine, e.g. one of 5 mM to 100 mM, 10 mM to 40 mM, 12 mM to 30 mM, 15 to 25 mM, or 18 to 22 mM. In some embodiments, the composition may comprise ˜20 mM histidine.

In some embodiments, the antigen-binding molecule is provided in a succinate buffer, i.e. a buffer comprising succinate ions. In some embodiments, the antigen-binding molecule is provided in a composition comprising succinate at a final concentration of 2 mM to 200 mM succinate, e.g. one of 5 mM to 100 mM, 10 mM to 40 mM, 12 mM to 30 mM, 15 to 25 mM, or 18 to 22 mM. In some embodiments, the composition may comprise ˜20 mM succinate. The buffer may comprise sodium succinate.

In some embodiments, the antigen-binding molecule is provided in a sodium phosphate buffer, i.e. a buffer comprising sodium and phosphate ions. In some embodiments, the antigen-binding molecule is provided in a composition comprising sodium phosphate at a final concentration of 2 mM to 200 mM sodium phosphate, e.g. one of 5 mM to 100 mM, 10 mM to 40 mM, 12 mM to 30 mM, 15 to 25 mM, or 18 to 22 mM. In some embodiments, the composition may comprise ˜20 mM sodium phosphate.

In some embodiments, the antigen-binding molecule is provided in a sodium acetate buffer, i.e. a buffer comprising sodium and acetate ions. In some embodiments, the antigen-binding molecule is provided in a composition comprising sodium acetate at a final concentration of 2 mM to 200 mM sodium acetate, e.g. one of 5 mM to 100 mM, 10 mM to 40 mM, 12 mM to 30 mM, 15 to 25 mM, or 18 to 22 mM. In some embodiments, the composition may comprise ˜20 mM sodium acetate.

In some embodiments, the antigen-binding molecule is provided in an arginine buffer, i.e. a buffer comprising arginine ions. In some embodiments, the antigen-binding molecule is provided in a composition comprising arginine at a final concentration of 1 mM to 250 mM arginine, e.g. one of 5 mM to 100 mM, 10 mM to 40 mM, 12 mM to 30 mM, 15 to 25 mM, or 18 to 22 mM. In some embodiments, the composition may comprise ˜20 mM arginine.

In some embodiments, the antigen-binding molecule is provided in a histidine-arginine buffer, i.e. a buffer comprising histidine and arginine ions. In some embodiments, the antigen-binding molecule is provided in a composition comprising histidine at a final concentration of 2 mM to 200 mM histidine, e.g. one of 5 mM to 100 mM, 10 mM to 40 mM, 12 mM to 30 mM, 15 to 25 mM, or 18 to 22 mM, and arginine at a final concentration of 1 mM to 300 mM arginine, e.g. one of 10 mM to 250 mM, 50 mM to 200 mM, 75 mM to 200 mM, 100 mM to 180 mM, or 125 to 175 mM. In some embodiments, the composition may comprise ˜20 mM histidine and ˜150 mM arginine.

In some embodiments, the composition comprising the antigen-binding molecule comprises an isotonicity agent. Isotonicity agents may be used to provide isotonic formulations. Examples of isotonicity agents include e.g. salts (e.g. sodium chloride, potassium chloride) and sugars (e.g. sucrose, glucose, trehalose).

In some embodiments, the antigen-binding molecule is provided in a composition comprising sodium chloride, i.e. sodium ions and chloride ions. The sodium chloride component of the composition may be provided at a final concentration of 1 mM to 250 mM sodium chloride, e.g. one of 10 mM to 250 mM, 50 mM to 200 mM, 75 mM to 200 mM, 100 mM to 180 mM, or 125 to 175 mM. In some embodiments, the composition may comprise ˜150 mM sodium chloride.

In some embodiments, the antigen-binding molecule is provided in a composition comprising methionine. The methionine component of the composition may be provided at a final concentration of 1 mM to 250 mM methionine, e.g. one of 10 mM to 250 mM, 50 mM to 200 mM, 75 mM to 200 mM, 100 mM to 180 mM, or 125 to 175 mM. In some embodiments, the composition may comprise ˜150 mM methionine.

In some embodiments, the antigen-binding molecule is provided in a composition comprising sucrose. The sucrose component of the composition may be provided at a final concentration (in weight by volume) of 2% to 20%, e.g. one of 2% to 15%, 3% to 12%, or 4% to 10%. In some embodiments, the composition may comprise ˜2, ˜4, ˜6, or ˜8% (w/v) sucrose. The sucrose component of the composition may be provided at a final concentration of 200 to 300 nM, e.g. ˜240 mM.

In some embodiments, the composition comprising the antigen-binding molecule comprises a surfactant. As used herein, a “surfactant” refers to an agent which lowers interfacial tension. The surfactant is preferably a non-ionic surfactant. Examples of surfactants include polysorbate (polysorbate-20, polysorbate-80), poloxamer (poloxamer-188) and Triton X-100. The surfactant is preferably present in the composition in the range from about 0.001% (w/v) to about 0.5% (w/V).

In some embodiments, the antigen-binding molecule is provided in a composition comprising polysorbate-20. The polysorbate-20 component of the composition may be provided at a final concentration (in weight by volume) of 0.001% to 0.1%, e.g. one of 0.002% to 0.08%, 0.006% to 0.05%, or 0.008% to 0.04%. In some embodiments, the composition may comprise ˜0.02% (w/v) polysorbate-20. In some embodiments, the composition may comprise ˜0.05% (w/v) polysorbate-20.

In some embodiments, the antigen-binding molecule is provided in a composition comprising polysorbate-80. The polysorbate-80 component of the composition may be provided at a final concentration (in weight by volume) of 0.001% to 0.1%, e.g. one of 0.002% to 0.08%, 0.006% to 0.05%, or 0.008% to 0.04%. In some embodiments, the composition may comprise ˜0.01% (w/v) polysorbate-80. In some embodiments, the composition may comprise ˜0.02% (w/v) polysorbate-80.

In some embodiments, the antigen-binding molecule is provided in a composition comprising:
2 mM to 200 mM (e.g. e.g. one of 5 mM to 100 mM, 10 mM to 40 mM, 12 mM to 30 mM, 15 to 25 mM, or 18 to 22 mM) histidine, more preferably ˜20 mM histidine;
2% to 20% (e.g. one of 2% to 15%, 3% to 12%, or 4% to 10%) sucrose (w/v), more preferably ˜8% (w/v) sucrose;
0.001% to 0.1% (e.g. one of 0.002% to 0.08%, 0.006% to 0.05%, or 0.008% to 0.04%) polysorbate-80 (w/v), more preferably ˜0.02% (w/v) polysorbate-80; and
pH 4.0 to 7.0 (e.g. one of pH 4.5 to pH 6.8, pH 4.6 to pH 6.4, pH 4.8 to pH 6.2, or pH 5.0 to pH 6.2), more preferably pH ˜5.5.

In some embodiments, the antigen-binding molecule is provided in a composition comprising:
2 mM to 200 mM (e.g. e.g. one of 5 mM to 100 mM, 10 mM to 40 mM, 12 mM to 30 mM, 15 to 25 mM, or 18 to 22 mM) histidine, more preferably ˜20 mM histidine;
2% to 20% (e.g. one of 2% to 15%, 3% to 12%, or 4% to 10%) sucrose (w/v), more preferably ˜8% (w/v) sucrose;
0.001% to 0.1% (e.g. one of 0.002% to 0.08%, 0.006% to 0.05%, or 0.008% to 0.04%) polysorbate-80 (w/v), more preferably ˜0.02% (w/v) polysorbate-80; and
pH 4.0 to 7.0 (e.g. one of pH 4.5 to pH 6.8, pH 4.6 to pH 6.4, pH 4.8 to pH 6.2, or pH 5.0 to pH 6.2), more preferably pH ˜5.8.

In some embodiments, the antigen-binding molecule is provided in a composition comprising:
2 mM to 200 mM (e.g. e.g. one of 5 mM to 100 mM, 10 mM to 40 mM, 12 mM to 30 mM, 15 to 25 mM, or 18 to 22 mM) histidine, more preferably ˜20 mM histidine;
2% to 20% (e.g. one of 1% to 15%, 2% to 10%, or 3% to 8%) sucrose (w/v), more preferably ˜4% (w/v) sucrose;
0.001% to 0.1% (e.g. one of 0.002% to 0.08%, 0.006% to 0.05%, or 0.008% to 0.04%) polysorbate-80 (w/v), more preferably ˜0.02% (w/v) polysorbate-80; and
pH 4.0 to 7.0 (e.g. one of pH 4.5 to pH 6.8, pH 4.6 to pH 6.4, pH 4.8 to pH 6.2, or pH 5.0 to pH 6.2), more preferably pH ˜5.8.

In some embodiments, the antigen-binding molecule is provided in a composition comprising:
2 mM to 200 mM (e.g. e.g. one of 5 mM to 100 mM, 10 mM to 40 mM, 12 mM to 30 mM, 15 to 25 mM, or 18 to 22 mM) histidine, more preferably ˜20 mM histidine;
0.2% to 20% (e.g. one of 0.5% to 15%, 0.75% to 10%, or 1% to 5%) sucrose (w/v), more preferably ˜2% (w/v) sucrose;
0.001% to 0.1% (e.g. one of 0.002% to 0.08%, 0.006% to 0.05%, or 0.008% to 0.04%) polysorbate-80 (w/v), more preferably ˜0.02% (w/v) polysorbate-80; and
pH 4.0 to 7.0 (e.g. one of pH 4.5 to pH 6.8, pH 4.6 to pH 6.4, pH 4.8 to pH 6.2, or pH 5.0 to pH 6.2), more preferably pH ˜5.8.

In some embodiments, the antigen-binding molecule is provided in a composition comprising:
2 mM to 200 mM (e.g. e.g. one of 5 mM to 100 mM, 10 mM to 40 mM, 12 mM to 30 mM, 15 to 25 mM, or 18 to 22 mM) histidine, more preferably ˜20 mM histidine;
2% to 20% (e.g. one of 2% to 15%, 3% to 12%, or 4% to 10%) sucrose (w/v), more preferably ˜8% (w/v) sucrose;
0.001% to 0.1% (e.g. one of 0.002% to 0.08%, 0.006% to 0.05%, or 0.008% to 0.04%) polysorbate-80 (w/v), more preferably ˜0.02% (w/v) polysorbate-80; and
pH 4.0 to 7.0 (e.g. one of pH 4.5 to pH 6.8, pH 4.6 to pH 6.4, pH 4.8 to pH 6.2, or pH 5.0 to pH 6.2), more preferably pH ˜6.3.

In some embodiments, the antigen-binding molecule is provided in a composition comprising:
2 mM to 200 mM (e.g. e.g. one of 5 mM to 100 mM, 10 mM to 40 mM, 12 mM to 30 mM, 15 to 25 mM, or 18 to 22 mM) histidine, more preferably ˜20 mM histidine;
2% to 20% (e.g. one of 2% to 15%, 3% to 12%, or 4% to 10%) sucrose (w/v), more preferably ˜8% (w/v) sucrose;
0.001% to 0.1% (e.g. one of 0.002% to 0.08%, 0.006% to 0.05%, or 0.008% to 0.04%) polysorbate-20 (w/v), more preferably ˜0.02% (w/v) polysorbate-20; and
pH 4.0 to 7.0 (e.g. one of pH 4.5 to pH 6.8, pH 4.6 to pH 6.4, pH 4.8 to pH 6.2, or pH 5.0 to pH 6.2), more preferably pH ˜5.8.

In some embodiments, the antigen-binding molecule is provided in a composition comprising:
2 mM to 200 mM (e.g. e.g. one of 5 mM to 100 mM, 10 mM to 40 mM, 12 mM to 30 mM, 15 to 25 mM, or 18 to 22 mM) acetate e.g. sodium acetate, more preferably ˜20 mM acetate;
2% to 20% (e.g. one of 2% to 15%, 3% to 12%, or 4% to 10%) sucrose (w/v), more preferably ˜8% (w/v) sucrose;
0.001% to 0.1% (e.g. one of 0.002% to 0.08%, 0.006% to 0.05%, or 0.008% to 0.04%) polysorbate-80 (w/v), more preferably ˜0.02% (w/v) polysorbate-80; and
pH 4.0 to 7.0 (e.g. one of pH 4.5 to pH 6.8, pH 4.6 to pH 6.4, pH 4.8 to pH 6.2, or pH 5.0 to pH 6.2), more preferably pH ˜5.5.

In some embodiments, the antigen-binding molecule is provided in a composition comprising:
2 mM to 200 mM (e.g. e.g. one of 5 mM to 100 mM, 10 mM to 40 mM, 12 mM to 30 mM, 15 to 25 mM, or 18 to 22 mM) succinate e.g. sodium succinate, more preferably ˜20 mM succinate;
2% to 20% (e.g. one of 2% to 15%, 3% to 12%, or 4% to 10%) sucrose (w/v), more preferably ˜8% (w/v) sucrose;
0.001% to 0.1% (e.g. one of 0.002% to 0.08%, 0.006% to 0.05%, or 0.008% to 0.04%) polysorbate-80 (w/v), more preferably ˜0.02% (w/v) polysorbate-80; and
pH 4.0 to 7.0 (e.g. one of pH 4.5 to pH 6.8, pH 4.6 to pH 6.4, pH 4.8 to pH 6.2, or pH 5.0 to pH 6.2), more preferably pH ˜5.5.

In some embodiments, the antigen-binding molecule is provided in a composition comprising:
2 mM to 200 mM (e.g. e.g. one of 5 mM to 100 mM, 10 mM to 40 mM, 12 mM to 30 mM, 15 to 25 mM, or 18 to 22 mM) histidine, more preferably ˜20 mM histidine;
0.001% to 0.1% (e.g. one of 0.002% to 0.08%, 0.006% to 0.05%, or 0.008% to 0.04%) polysorbate-80 (w/v), more preferably ˜0.02% (w/v) polysorbate-80; and
pH 4.0 to 7.0 (e.g. one of pH 4.5 to pH 6.8, pH 4.6 to pH 6.4, pH 4.8 to pH 6.2, or pH 5.0 to pH 6.2), more preferably pH ˜5.8.

In some embodiments, the antigen-binding molecule is provided in a composition comprising:
2 mM to 200 mM (e.g. e.g. one of 5 mM to 100 mM, 10 mM to 40 mM, 12 mM to 30 mM, 15 to 25 mM, or 18 to 22 mM) histidine, more preferably ˜20 mM histidine;
1 mM to 250 mM (e.g. one of 10 mM to 250 mM, 50 mM to 200 mM, 75 mM to 200 mM, 100 mM to 180 mM, or 125 to 175 mM) sodium chloride, more preferably ˜150 mM sodium chloride; and
pH 4.0 to 7.0 (e.g. one of pH 4.5 to pH 6.8, pH 4.6 to pH 6.4, pH 4.8 to pH 6.2, or pH 5.0 to pH 6.2), more preferably pH ˜5.8.

The composition may comprise about 0.025 mg/mL to about 100 mg/mL antigen-binding molecule. The composition may comprise about 0.05 mg/mL to about 80 mg/mL antigen-binding molecule. The composition may comprise about 0.06 mg/mL to about 70 mg/mL antigen-binding molecule. The composition may comprise about 0.07 mg/mL to about 60 mg/mL antigen-binding molecule. The composition may comprise about 0.07 mg/mL to about 50 mg/mL antigen-binding molecule.

The antigen-binding molecule may be formulated at a concentration of about 30 mg/mL, about 35 mg/mL, about 40 mg/mL, about 45 mg/mL, about 50 mg/mL, about 55 mg/mL, about 60 mg/mL, about 65 mg/mL, or about 70 mg/mL, or any ranges therein, e.g. in a composition according to the present disclosure. The antigen-binding molecule may be formulated at a concentration of about 50 mg/mL, e.g. in a composition according to the present disclosure.

As used herein, “about” refers to the specified concentration, and plus or minus 10% of that concentration. For example, a concentration of “about 50 mg/mL” refers to a concentration ranging from 45 mg/mL to 55 mg/mL, including a concentration of 50 mg/mL.

The antigen-binding molecule may be formulated at a concentration of at least 0.05 mg/ml, at least 0.1 mg/mL, at least 0.15 mg/mL, at least 0.2 mg/mL, at least 0.25 mg/mL, at least 0.3 mg/mL, at least 0.35 mg/mL, at least 0.4 mg/mL, at least 0.5 mg/mL, at least 0.8 mg/mL, at least 1 mg/mL, at least 1.4 mg/mL, at least 2 mg/mL, at least 2.4 mg/mL, at least 4 mg/mL, at least 6 mg/mL, at least 8 mg/mL, at least 10 mg/mL, at least 12 mg/mL, at least 14 mg/mL, or at least 16 mg/mL, e.g. in a composition according to the present disclosure.

The 50 mg/mL antigen-binding molecule solution may be diluted before administration using any suitable excipient. In some embodiments, the 50 mg/mL antigen-binding molecule solution is diluted for administration in 5% dextrose. In some embodiments, the 50 mg/mL antigen-binding molecule solution is diluted for administration to a concentration of at least 0.07 mg/mL, at least 0.14 mg/mL, at least 0.21 mg/mL, at least 0.35 mg/mL, at least 0.42 mg/mL, at least 0.8 mg/mL, at least 1.4 mg/ml, at least 2.4 mg/mL, e.g. in a volume of 50 mL. In some embodiments, the 50 mg/mL antigen-binding molecule solution is diluted for administration to a concentration of at least 2.4 mg/mL, at least 4 mg/mL, at least 8 mg/mL, at least 12 mg/mL, or at least 16 mg/mL, e.g. in a volume of 100 mL. In some embodiments, the diluted antigen-binding molecule solution is then administered to a subject, e.g. via intravenous administration, e.g. using a dose/dosing regime according to the present disclosure, e.g. to treat a disease/condition according to the present disclosure. In some embodiments, the diluted antigen-binding molecule solution is administered to the subject within 48 hours from the initial dilution step.

The antigen-binding molecule according to the present disclosure, e.g. using the doses and/or dosage regimes described herein, may be administered in combination with an agent capable of inhibiting signalling mediated by PD-1. The agent capable of inhibiting signalling mediated by PD-1 may be a PD-1- or PD-L1-targeted agent. The agent capable of inhibiting signalling mediated by PD-1 may e.g. be an antibody capable of binding to PD-1 or PD-L1 and inhibiting PD-1-mediated signalling. In some embodiments the agent is an antagonist anti-PD-1 antibody. In some embodiments the agent is an antagonist anti-PD-L1 antibody. In some embodiments the agent is Pembrolizumab (Keytruda), Nivolumab (Opdivo), Cemiplimab (Libtayo), Atezolizumab (Tecentriq), Avelumab (Bavencio), or Durvalumab (Imfinzi). Such agents may be prepared and administered according to the drug preparation instructions provided with the agent(s).

### Therapeutic and Prophylactic Applications

The antigen-binding molecules, polypeptides, CARs, nucleic acids, expression vectors, cells and compositions described herein find use in therapeutic and prophylactic methods.

The present invention provides an antigen-binding molecule, polypeptide, CAR, nucleic acid (or plurality thereof), expression vector (or plurality thereof), cell or composition described herein for use in a method of medical treatment or prophylaxis. Also provided is the use of an antigen-binding molecule, polypeptide, CAR, nucleic acid (or plurality thereof), expression vector (or plurality thereof), cell or composition described herein in the manufacture of a medicament for treating or preventing a disease or condition.

Also provided is a method of treating or preventing a disease or condition, comprising administering to a subject a therapeutically or prophylactically effective amount of an antigen-binding molecule, polypeptide, CAR, nucleic acid (or plurality thereof), expression vector (or plurality thereof), cell or composition described herein. All therapeutic and prophylactic methods/uses described herein may be performed on a subject.

The methods may be effective to reduce the development or progression of a disease/condition, alleviation of the symptoms of a disease/condition or reduction in the pathology of a disease/condition. The methods may be effective to prevent progression of the disease/condition, e.g. to prevent worsening of, or to slow the rate of development of, the disease/condition. In some embodiments the methods may lead to an improvement in the disease/condition, e.g. a reduction in the symptoms of the disease/condition or reduction in some other correlate of the severity/activity of the disease/condition. In some embodiments the methods may prevent development of the disease/condition to a later stage (e.g. a chronic stage or metastasis).

It will be appreciated that the articles of the present invention may be used for the treatment/prevention of any disease/condition that would derive therapeutic or prophylactic benefit from a reduction in the number and/or activity of cells expressing VISTA (e.g. MDSCs). It will also be clear that the therapeutic and prophylactic utility of the present invention extends to essentially any disease/condition which would benefit from a reduction in the number or activity of MDSCs and/or other cells expressing VISTA, e.g. tumor-associated macrophages (TAMs) and neutrophils. Antagonism of VISTA effectively releases effector immune cells from suppression by MDSCs and/or other cells expressing VISTA.

For example, the disease/condition may be a disease/condition in which cells expressing VISTA (e.g. MDSCs) are pathologically implicated, e.g. a disease/condition in which an increased number/proportion of cells expressing VISTA (e.g. MDSCs) is positively associated with the onset, development or progression of the disease/condition, and/or severity of one or more symptoms of the disease/condition, or for which an increased number/proportion of cells expressing VISTA (e.g. MDSCs), is a risk factor for the onset, development or progression of the disease/condition.

In some embodiments, the disease/condition to be treated/prevented in accordance with the present invention is a disease/condition characterised by an increase in the number/proportion/activity of cells expressing VISTA (e.g. MDSCs), e.g. as compared to the number/proportion/activity of cells expressing VISTA (e.g. MDSCs) in the absence of the disease/condition.

In some embodiments, a subject may be selected for treatment described herein based on the detection of an increase in the number/proportion/activity of cells expressing VISTA (e.g. MDSCs), e.g. in the periphery, or in an organ/tissue which is affected by the disease/condition (e.g. an organ/tissue in which the symptoms of the disease/condition manifest), or by the presence of cells expressing VISTA (e.g. MDSCs or tumor-associated macrophages) in a tumor. The disease/condition may affect any tissue or organ or organ system. In some embodiments the disease/condition may affect several tissues/organs/organ systems.

In some embodiments a subject may be selected for therapy/prophylaxis in accordance with the present invention based on determination that the subject has an increase in the number/proportion/activity of cells expressing VISTA (e.g. MDSCs) in the periphery or in an organ/tissue relative to the number/proportion/activity of such cells in a healthy subject, or based on determination that the subject has a tumor comprising cells expressing VISTA (e.g. MDSCs).

In some embodiments the disease/condition to be treated/prevented is a cancer.

It will be appreciated that the antigen-binding molecules are useful for the treatment of cancers in general, because antigen-binding molecules of the present invention are useful to release effector immune cells from MDSC-mediated suppression or suppression by cells expressing VISTA, and thereby enhance the anticancer immune response.

The cancer may be any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

MDSCs are elevated in advanced colorectal cancer (Toor et al, Front Immunol. 2016; 7:560). MDSCs are also observed in breast cancer, and the percentage of MDSCs in the peripheral blood is increased in patients with later stage breast cancer (Markowitz et al, Breast Cancer Res Treat. 2013 July; 140(1): 13-21). MDSC abundance is also correlated with poor prognosis in solid tumors (Charoentong et al, Cell Rep. 2017 Jan. 3; 18(1): 248-262), and MDSCs are enriched in liver cancer models (Connolly et al., J Leukoc Biol. (2010) 87(4): 713-25). Prostate and breast carcinomas, melanomas, colorectal cancer and Lewis lung carcinoma have been reported to produce chemokines which attract MDSCs and contribute to immune suppression (Umansky et al., Vaccines (Basel) (2016) 4(4): 36)), and MDSCs in pancreatic cancer patients have been positively correlated with tumor burden (Xu et al., Hepatobiliary Pancreat Dis Int. (2016) 15(1): 99-105). VISTA has also been reported to be a target for the treatment of ovarian cancer (see e.g. U.S. Pat. No. 9,631,018 B2) and lymphoma (see e.g. WO 2017/023749 A1).

Blando et al. Proc Natl Acad Sci USA. (2019) 116(5): 1692-1697 recently reported significant infiltration of VISTA-expressing myeloid cells in pancreatic cancer, and expansion of VISTA-expressing myeloid cells has been observed following treatment with CTLA4 antagonist in prostate cancer, and both pre- and post-treatment with PD-L1 antagonist in melanoma.

In some embodiments, a cancer is selected from: a cancer comprising cells expressing VISTA, a cancer comprising infiltration of cells expressing VISTA, a cancer comprising cancer cells expressing VISTA, a hematological cancer, leukemia, acute myeloid leukemia, lymphoma, B cell lymphoma, T cell lymphoma, multiple myeloma, mesothelioma, a solid tumor, lung cancer, non-small cell lung carcinoma (NSCLC), gastric cancer, gastric carcinoma, colorectal cancer, colorectal carcinoma, colorectal adenocarcinoma, uterine cancer, uterine corpus endometrial carcinoma, breast cancer, triple negative breast cancer (TBNC), triple negative breast invasive carcinoma, invasive ductal carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, bladder cancer, prostate cancer and/or prostate adenocarcinoma.

In some embodiments the cancer is colorectal cancer (e.g. colon carcinoma, colon adenocarcinoma), pancreatic cancer, breast cancer (e.g. triple-negative breast cancer; TBNC), invasive ductal carcinoma, liver cancer, prostate cancer, ovarian cancer, head and neck cancer, leukemia (e.g. T cell leukemia), lymphoma, melanoma, thymoma, lung cancer, non-small cell lung cancer (NSCLC), mesothelioma, bladder cancer, uterine cancer, and/or a solid tumor.

In some embodiments the cancer is an advanced, unresectable or metastatic cancer.

In some embodiments the cancer is metastatic triple-negative breast cancer (TBNC). The TBNC may be defined as estrogen receptor (ER)-negative, progesterone receptor (PR)-negative, and human epidermal growth factor 2 (HER2)-negative; or ER-negative, PR-negative and HER2-positive. The cancer may be defined as having estrogen receptor (ER) expression ≤10% and progesterone receptor (PR) expression ≤10%.

In some embodiments the cancer is metastatic and/or advanced non-small cell lung cancer (NSCLC), e.g. locally advances and unresectable. In some embodiments the cancer, e.g. NSCLC, does not comprise an activating mutation of epidermal growth factor receptor (EGFR) or anaplastic lymphoma kinase (ALK). The treatment/prevention may be aimed at one or more of: delaying/preventing the onset/progression of symptoms of the cancer, reducing the severity of symptoms of the cancer, reducing the survival/growth/invasion/metastasis of cells of the cancer, reducing the number of cells of the cancer and/or increasing survival of the subject.

The articles of the present disclosure (i.e. antigen-binding molecules, compositions, etc.) are particularly useful for the treatment/prevention of diseases/conditions (e.g. cancers) characterised by the presence of cells expressing VISTA and/or diseases/conditions (e.g. cancers) characterised by signalling mediated by a complex comprising VISTA.

A cancer characterised by the presence of cells expressing VISTA may comprise cancerous cells expressing VISTA. That is, cells of the cancer may express VISTA. It will be appreciated that in embodiments herein, cancers comprising cells having specified characteristics may be or comprise tumors comprising cells having those characteristics.

Alternatively, or additionally, a cancer characterised by ‘the presence of’ cells expressing VISTA may be characterised by the presence of cells expressing VISTA in the vicinity of (i.e. proximal to) cells of the cancer. The cancer may comprise infiltration of cells expressing VISTA. The cancer may comprise a tumor displaying infiltration of cells expressing VISTA. In connection with such embodiments, the cells expressing VISTA are not necessarily cells of the cancer, and may e.g. be non-cancerous cells. In some embodiments, the cells are immune cells. The immune cells expressing VISTA may be or comprise cells of hematopoietic origin, e.g. neutrophils, eosinophils, basophils, dendritic cells, lymphocytes, or monocytes. In some embodiments, the immune cells expressing VISTA may be or comprise lymphocytes, e.g. a T cells, B cells, NK cells, NKT cells, innate lymphoid cells (ILCs), or precursors thereof. In some embodiments, the immune cells expressing VISTA are effector immune cells (e.g. CD8+ T cells, CD8+ cytotoxic T lymphocytes (CD8+ CTLs), CD4+ T cells, CD4+ T helper cells, NK cells, IFNγ-producing cells, memory T cells, central memory T cells, antigen-experienced T cells and/or CD45RO+ T cells). In some embodiments, the immune cells expressing VISTA are antigen-presenting cells (APCs), macrophages, dendritic cells, T cells (e.g. CD8+ T cells) and/or MDSCs. In embodiments wherein the cancer comprises infiltration of immune cells expressing VISTA, or wherein the cancer comprises a tumor displaying infiltration of such immune cells, the immune cells may be referred to as tumor-infiltrating immune cells.

For example, a cancer characterised by the presence of cells expressing VISTA may comprise a tumor comprising cells (e.g. non-cancerous cells, e.g. tumor-infiltrating immune cells) expressing VISTA.

Cancers, cancer cells and/or tumors comprising cells expressing VISTA may be described as VISTA-positive, or VISTA IHC+.

It will be appreciated that cells that are said to be ‘in the vicinity of’ or ‘proximal to’ cells of a cancer are provided in close physical proximity to cells of a cancer, e.g. in vivo in a subject having such a cancer. In some embodiments, cells which are in the vicinity of/proximal to cells of a cancer are comprised in the same organ/tissue as cells of the cancer. In some embodiments, cells which are in the vicinity of/proximal to cells of a cancer are comprised in a tumor of the cancer. In some embodiments, cells which are in the vicinity of/proximal to cells of a cancer are in contact with cells of the cancer.

In some embodiments, the cancer to be treated/prevented comprises cells expressing VISTA. In some embodiments, the cancer to be treated/prevented comprises cancer cells expressing VISTA. In some embodiments, the cells expressing VISTA are MDSCs (e.g. g-MDSCs and/or m-MDSCs). In some embodiments, the cancer comprises a tumor comprising cells expressing VISTA (e.g. MDSCs). In some embodiments, the cancer to be treated/prevented comprises a tumor comprising MDSCs. In some embodiments, the cancer to be treated/prevented comprises infiltration of cells expressing VISTA (e.g. MDSCs). In some embodiments, the cancer to be treated/prevented comprises a tumor displaying infiltration of cells expressing VISTA (e.g. MDSCs).

In some embodiments, the cancer to be treated/prevented comprises a tumor comprising a population of CD45+ cells comprising greater than 1%, e.g. ≥2%, ≥5%, ≥10%, ≥15%, ≥20%, ≥25% or ≥30% MDSCs (e.g. as determined by immunoprofiling of the tumor).

VISTA-binding antigen-binding molecules described herein may inhibit interaction between VISTA and interaction partners for VISTA (e.g. LRIG1, VSIG3, PSGL-1, VSIG8). Inhibition of interaction between VISTA and its interaction partners using the antigen-binding molecules described herein is moreover demonstrated to release effector immune cells from MDSC-mediated suppression of their activity.

Accordingly, the articles of the present disclosure (i.e. antigen-binding molecules, compositions, etc.) are particularly useful for the treatment/prevention of diseases/conditions (e.g. cancers) characterised by the presence of cells expressing an interaction partner for VISTA (e.g. LRIG1, VSIG3, PSGL-1, VSIG8), and/or diseases/conditions (e.g. cancers) characterised by signalling mediated by a complex comprising VISTA and an interaction partner for VISTA (e.g. LRIG1, VSIG3, PSGL-1, VSIG8).

A cancer characterised by the presence of cells expressing an interaction partner for VISTA (e.g. LRIG1, VSIG3, PSGL-1, VSIG8) may comprise cancerous cells expressing the interaction partner for VISTA. That is, cells of the cancer may express the interaction partner for VISTA.

Alternatively, or additionally, a cancer characterised by ‘the presence of’ cells expressing an interaction partner for VISTA (e.g. LRIG1, VSIG3, PSGL-1, VSIG8) may be characterised by the presence of cells expressing the interaction partner for VISTA in the vicinity of (i.e. proximal to) cells of the cancer. The cancer may comprise infiltration of cells expressing an interaction partner for VISTA. The cancer may comprise a tumor displaying infiltration of cells expressing an interaction partner for VISTA. In connection with such embodiments, the cells expressing the interaction partner for VISTA are not necessarily cells of the cancer, and may e.g. be non-cancerous cells. In some embodiments, the cells are immune cells. The immune cells expressing an interaction partner for VISTA (e.g. LRIG1, VSIG3, PSGL-1, VSIG8) may be or comprise cells of hematopoietic origin, e.g. neutrophils, eosinophils, basophils, dendritic cells, lymphocytes, or monocytes. In some embodiments, the immune cells expressing an interaction partner for VISTA may be or comprise lymphocytes, e.g. a T cells, B cells, NK cells, NKT cells, innate lymphoid cells (ILCs), or precursors thereof. In some embodiments, the immune cells expressing an interaction partner for VISTA are effector immune cells (e.g. CD8+ T cells, CD8+ cytotoxic T lymphocytes (CD8+ CTLs), CD4+ T cells, CD4+ T helper cells, NK cells, IFNγ-producing cells, memory T cells, central memory T cells, antigen-experienced T cells and/or CD45RO+ T cells). In some embodiments, the immune cells expressing an interaction partner for VISTA are antigen-presenting cells (APCs), macrophages, dendritic cells, T cells (e.g. CD8+ T cells) and/or MDSCs. In embodiments wherein the cancer comprises infiltration of immune cells expressing an interaction partner for VISTA, or wherein the cancer comprises a tumor displaying infiltration of such immune cells, the immune cells may be referred to as tumor-infiltrating immune cells.

For example, a cancer characterised by the presence of cells expressing an interaction partner for VISTA (e.g. LRIG1, VSIG3, PSGL-1, VSIG8) may comprise a tumor comprising cells (e.g. non-cancerous cells, e.g. tumor-infiltrating immune cells) expressing the interaction partner for VISTA.

It will be appreciated that in embodiments herein, cancers comprising cells having specified characteristics may be or comprise tumors comprising cells having those characteristics. In some embodiments, the cancer to be treated/prevented comprises a tumor characterised by the presence of cells (which may e.g. be non-cancerous cells, e.g. tumor-infiltrating immune cells) expressing an interaction partner for VISTA. In some embodiments, the cancer to be treated/prevented comprises a tumor comprising cells expressing an interaction partner for VISTA. In some embodiments, the cancer to be treated/prevented comprises infiltration of cells expressing an interaction partner for VISTA. In some embodiments, the cancer to be treated/prevented comprises a tumor displaying infiltration of cells expressing an interaction partner for VISTA.

In some embodiments, cells expressing an interaction partner for VISTA may display increased expression of the interaction partner, relative to a reference level of expression, e.g. by cells of the same type (e.g. from the same organ/issue) under non-pathological conditions. In some embodiments, cells expressing an interaction partner for VISTA may overexpress the interaction partner for VISTA. Such cells may express the relevant molecule at a level which is greater than the level of expression by equivalent non-cancerous cells/non-tumor tissue. In some embodiments, the cancer to be treated/prevented is characterised by signalling mediated by a complex comprising VISTA and an interaction partner for VISTA. In some embodiments, the cancer to be treated/prevented is characterised by an elevated level of signalling mediated by a complex comprising VISTA and an interaction partner for VISTA, i.e. as compared to the level of signalling by the relevant complex in the absence of the cancer.

In some embodiments, the cancer to be treated/prevented is characterised by the presence of (i) cells expressing VISTA, and (ii) cells expressing an interaction partner for VISTA. In some embodiments, the cancer to be treated/prevented comprises (i) cells expressing VISTA, and (ii) cells expressing an interaction partner for VISTA. In some embodiments, the cancer to be treated/prevented comprises a tumor characterised by the presence of (i) cells expressing VISTA, and (ii) cells expressing an interaction partner for VISTA. In some embodiments, the cancer to be treated/prevented comprises a tumor comprising (i) cells expressing VISTA, and (ii) cells expressing an interaction partner for VISTA. In some embodiments, the cancer to be treated/prevented comprises infiltration of (i) cells expressing VISTA, and (ii) cells expressing an interaction partner for VISTA. In some embodiments, the cancer to be treated/prevented comprises a tumor displaying infiltration of (i) cells expressing VISTA, and (ii) cells expressing an interaction partner for VISTA. In some embodiments, the cancer to be treated/prevented is a cancer characterised by signalling mediated by a complex comprising VISTA and an interaction partner for VISTA. In some embodiments, the cancer to be treated/prevented is a cancer characterised by an elevated level of signalling mediated by a complex comprising VISTA and an interaction partner for

VISTA, e.g. as compared to the level of signalling by the complex in the relevant cell type/tissue/organ in the absence of cancer.

In some embodiments, a subject may be selected for treatment described herein based on the detection of a cancer comprising cells expressing VISTA/an interaction partner for VISTA (e.g. MDSCs), or detection of a tumor comprising cells expressing VISTA/an interaction partner for VISTA (e.g. MDSCs), e.g. in a sample obtained from the subject, e.g. to characterise a cancer/tumor as described above.

In some embodiments the disease/condition in which the VISTA-expressing cells are pathologically implicated is an infectious disease, e.g. bacterial, viral, fungal, or parasitic infection. In some embodiments it may be particularly desirable to treat chronic/persistent infections, e.g. where such infections are associated with T cell dysfunction or T cell exhaustion. It is well established that T cell exhaustion is a state of T cell dysfunction that arises during many chronic infections (including viral, bacterial and parasitic), as well as in cancer (Wherry Nature Immunology Vol. 12, No. 6, p 492-499, June 2011).

Examples of bacterial infections that may be treated include infection by Bacillus spp., Bordetella pertussis, Clostridium spp., Corynebacterium spp., Vibrio chloerae, Staphylococcus spp., Streptococcus spp. Escherichia, Klebsiella, Proteus, Yersinia, Erwina, Salmonella, Listeria sp, Helicobacter pylori, mycobacteria (e.g. Mycobacterium tuberculosis) and Pseudomonas aeruginosa. For example, the bacterial infection may be sepsis or tuberculosis. Examples of viral infections that may be treated include infection by influenza virus, measles virus, hepatitis B virus (HBV), hepatitis C virus (HCV), human immunodeficiency virus (HIV), lymphocytic choriomeningitis virus (LCMV), Herpes simplex virus and human papilloma virus (HPV). Examples of fungal infections that may be treated include infection by Alternaria sp, Aspergillus sp, Candida sp and Histoplasma sp. The fungal infection may be fungal sepsis or histoplasmosis. Examples of parasitic infections that may be treated include infection by Plasmodium species (e.g. Plasmodium falciparum, Plasmodium yoeli, Plasmodium ovale, Plasmodium vivax, or Plasmodium chabaudi chabaudi). The parasitic infection may be a disease such as malaria, leishmaniasis and toxoplasmosis.

In some embodiments the antigen-binding molecule exerts its therapeutic/prophylactic effect via a molecular mechanism which does not involve an Fc region-mediated effector function (e.g. ADCC, ADCP, CDC). In some embodiments the molecular mechanism does not involve binding of the antigen-binding molecule to an Fcγ receptor (e.g. one or more of FcγRI, FcγRIIa, FcγRIIb, FcγRIIc, FcγRIIIa and FcγRIIIb). In some embodiments the molecular mechanism does not involve binding of the antigen-binding molecule to a complement protein (e.g. C1q).

In some embodiments (e.g. embodiments wherein the antigen-binding molecule lacks an Fc region, or embodiments wherein the antigen-binding molecule comprises an Fc region which is not able to induce an Fc-mediated antibody effector function), the treatment does not induce/increase killing of VISTA-expressing cells. In some embodiments the treatment does not reduce the number/proportion of VISTA-expressing cells.

In some embodiments the treatment (i) inhibits VISTA-mediated signalling, and (ii) does not induce/increase killing of VISTA-expressing cells. In some embodiments the treatment (i) inhibits VISTA-mediated signalling, and (ii) does not reduce the number/proportion of VISTA-expressing cells.

Administration of the articles of the present invention is preferably in a “therapeutically effective” or “prophylactically effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Administration may be alone or in combination with other treatments, either simultaneously or sequentially dependent upon the condition to be treated. The antigen-binding molecule or composition described herein and a therapeutic agent may be administered simultaneously or sequentially.

In some embodiments, the methods comprise additional therapeutic or prophylactic intervention, e.g. for the treatment/prevention of a cancer. In some embodiments, the therapeutic or prophylactic intervention is selected from chemotherapy, immunotherapy, radiotherapy, surgery, vaccination and/or hormone therapy. In some embodiments, the therapeutic or prophylactic intervention comprises leukapheresis. In some embodiments the therapeutic or prophylactic intervention comprises a stem cell transplant.

In some embodiments the antigen-binding molecule is administered in combination with an agent capable of inhibiting signalling mediated by an immune checkpoint molecule other than VISTA. In some embodiments the immune checkpoint molecule is e.g. PD-1, CTLA-4, LAG-3, TIM-3, TIGIT or BTLA. In some embodiments the antigen-binding molecule is administered in combination with an agent capable of promoting signalling mediated by a costimulatory receptor. In some embodiments the costimulatory receptor is e.g. CD28, CD80, CD40L, CD86, OX40, 4-1BB, CD27 or ICOS.

Accordingly, the invention provides compositions comprising an article according to the present invention (e.g. an antigen-binding molecule according to the invention) and an agent capable of inhibiting signalling mediated by an immune checkpoint molecule other than VISTA. Also provided are compositions comprising the articles of the present invention and an agent capable of promoting signalling mediated by a costimulatory receptor. Also provided is the use of such compositions in methods of medical treatment and prophylaxis of diseases/conditions described herein.

Also provided are methods for treating/preventing diseases/conditions described herein comprising administering an article according to the present invention (e.g. an antigen-binding molecule according to the invention) and an agent capable of inhibiting signalling mediated by an immune checkpoint molecule other than VISTA. Also provided are methods for treating/preventing diseases/conditions described herein comprising administering an article according to the present invention (e.g. an antigen-binding molecule according to the invention) and an agent capable of promoting signalling mediated by a costimulatory receptor.

Agents capable of inhibiting signalling mediated by immune checkpoint molecules are known in the art, and include e.g. antibodies capable of binding to immune checkpoint molecules or their ligands, and inhibiting signalling mediated by the immune checkpoint molecule. Other agents capable of inhibiting signalling mediated by an immune checkpoint molecule include agents capable of reducing gene/protein expression of the immune checkpoint molecule or a ligand for the immune checkpoint molecule (e.g. through inhibiting transcription of the gene(s) encoding the immune checkpoint molecule/ligand, inhibiting post-transcriptional processing of RNA encoding the immune checkpoint molecule/ligand, reducing stability of RNA encoding the immune checkpoint molecule/ligand, promoting degradation of RNA encoding the immune checkpoint molecule/ligand, inhibiting post-translational processing of the immune checkpoint molecule/ligand, reducing stability the immune checkpoint molecule/ligand, or promoting degradation of the immune checkpoint molecule/ligand), and small molecule inhibitors.

Agents capable of promoting signalling mediated by costimulatory receptors are known in the art, and include e.g. agonist antibodies capable of binding to costimulatory receptors and triggering or increasing signalling mediated by the costimulatory receptor. Other agents capable of promoting signalling mediated by costimulatory receptors include agents capable of increasing gene/protein expression of the costimulatory receptor or a ligand for the costimulatory receptor (e.g. through promoting transcription of the gene(s) encoding the costimulatory receptor/ligand, promoting post-transcriptional processing of RNA encoding the costimulatory receptor/ligand, increasing stability of RNA encoding the costimulatory receptor/ligand, inhibiting degradation of RNA encoding the costimulatory receptor/ligand, promoting post-translational processing of the costimulatory receptor/ligand, increasing stability the costimulatory receptor/ligand, or inhibiting degradation of the costimulatory receptor/ligand), and small molecule agonists.

Immune suppression by VISTA-expressing MDSCs has been implicated in the failure of, and development of resistance to, treatment with agents capable of inhibiting signalling mediated by an immune checkpoint molecules. Gao et al., Nature Medicine (2017) 23:551-555 recently suggested that VISTA may be a compensatory inhibitory pathway in prostate tumors after ipilimumab (i.e. anti-CTLA-4 antibody) therapy.

In particular embodiments the antigen-binding molecule of the present invention is administered in combination with an agent capable of inhibiting signalling mediated by PD-1. The agent capable of inhibiting signalling mediated by PD-1 may be a PD-1- or PD-L1-targeted agent. The agent capable of inhibiting signalling mediated by PD-1 may e.g. be an antibody capable of binding to PD-1 or PD-L1 and inhibiting PD-1-mediated signalling. In some embodiments the agent is an antagonist anti-PD-1 antibody. In some embodiments the agent is an antagonist anti-PD-L1 antibody.

In some embodiments, the antigen-binding molecule of the present invention is administered in combination with an agent capable of inhibiting signalling mediated by CTLA-4. The agent capable of inhibiting signalling mediated by CTLA-4 may be a CTLA-4-targeted agent, or an agent targeted against a ligand for CTLA-4 such as CD80 or CD86. In some embodiments, the agent capable of inhibiting signalling mediated by CTLA-4 may e.g. be an antibody capable of binding to CTLA-4, CD80 or CD86 and inhibiting CTLA-4-mediated signalling.

In some embodiments, the antigen-binding molecule of the present invention is administered in combination with an agent capable of inhibiting signalling mediated by LAG-3. The agent capable of inhibiting signalling mediated by LAG-3 may be a LAG-3-targeted agent, or an agent targeted against a ligand for LAG-3 such as MHC class II. In some embodiments, the agent capable of inhibiting signalling mediated by LAG-3 may e.g. be an antibody capable of binding to LAG-3 or MHC class II and inhibiting LAG-3-mediated signalling.

In some embodiments, the antigen-binding molecule of the present invention is administered in combination with an agent capable of inhibiting signalling mediated by TIM-3. The agent capable of inhibiting signalling mediated by TIM-3 may be a TIM-3-targeted agent, or an agent targeted against a ligand for TIM-3 such as Galectin 9. In some embodiments, the agent capable of inhibiting signalling mediated by TIM-3 may e.g. be an antibody capable of binding to TIM-3 or Galectin 9 and inhibiting TIM-3-mediated signalling.

In some embodiments, the antigen-binding molecule of the present invention is administered in combination with an agent capable of inhibiting signalling mediated by TIGIT. The agent capable of inhibiting signalling mediated by TIGIT may be a TIGIT-targeted agent, or an agent targeted against a ligand for TIGIT such as CD113, CD112 or CD155. In some embodiments, the agent capable of inhibiting signalling mediated by TIGIT may e.g. be an antibody capable of binding to TIGIT, CD113, CD112 or CD155 and inhibiting TIGIT-mediated signalling.

In some embodiments, the antigen-binding molecule of the present invention is administered in combination with an agent capable of inhibiting signalling mediated by BTLA. The agent capable of inhibiting signalling mediated by BTLA may be a BTLA-targeted agent, or an agent targeted against a ligand for BTLA such as HVEM. In some embodiments, the agent capable of inhibiting signalling mediated by BTLA may e.g. be an antibody capable of binding to BTLA or HVEM and inhibiting BTLA-mediated signalling.

In some embodiments methods employing a combination of an antigen-binding molecule of the present invention and an agent capable of inhibiting signalling mediated by an immune checkpoint molecule (e.g. PD-1 and/or PD-L1) provide an improved treatment effect as compared to the effect observed when either agent is used as a monotherapy. In some embodiments the combination of an antigen-binding molecule of the present invention and an agent capable of inhibiting signalling mediated by an immune checkpoint molecule (e.g. PD-1 and/or PD-L1) provide a synergistic (i.e. super-additive) treatment effect.

In some embodiments, treatment with a combination comprising (i) an antigen-binding molecule of the present invention and (ii) an agent capable of inhibiting signalling mediated by an immune checkpoint molecule (e.g. PD-1 and/or PD-L1) may be associated with one or more of:
an improved treatment effect as compared to the treatment effect observed with either component of the combination used alone;
a treatment effect which is synergistic (i.e. super-additive) as compared to the treatment effect observed with either component of the combination used alone;
increased inhibition of tumor growth as compared to inhibition of tumor growth by either component of the combination used alone;
inhibition of tumor growth which is synergistic (i.e. super-additive) as compared to inhibition of tumor growth by either component of the combination used alone;
greater reduction in the number/activity of suppressor immune cells as compared to reduction of the number/activity of suppressor immune cells by either component of the combination used alone;
reduction in the number/activity of suppressor immune cells which is synergistic (i.e. super-additive) as compared to reduction of the number/activity of suppressor immune cells by either component of the combination used alone;
greater reduction of proliferation of suppressor immune cells as compared to reduction proliferation of suppressor immune cells by either component of the combination used alone reduction of proliferation of suppressor immune cells which is synergistic (i.e. super-additive) as compared to reduction proliferation of suppressor immune cells by either component of the combination used alone;
greater reduction in the proportion of suppressor immune cells within a population of cells (e.g. CD45+ cells, e.g. CD45+ cells obtained from a tumor) as compared to the reduction of the proportion of suppressor immune cells by either component of the combination used alone; and
reduction in the proportion of suppressor immune cells within a population of cells (e.g. CD45+ cells, e.g. CD45+ cells obtained from a tumor) which is synergistic (i.e. super-additive) as compared to the reduction of the proportion of suppressor immune cells by either component of the combination used alone.

In some embodiments an agent capable of inhibiting signalling mediated by an immune checkpoint molecule (e.g. PD-1 and/or PD-L1) is Pembrolizumab (Keytruda; MK-3475, lambrolizumab), Nivolumab (Opdivo), Cemiplimab (Libtayo), Atezolizumab (Tecentriq), Avelumab (Bavencio), or Durvalumab (Imfinzi).

Simultaneous administration refers to administration of the antigen-binding molecule, polypeptide, CAR, nucleic acid (or plurality thereof), expression vector (or plurality thereof), cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one of the antigen-binding molecule/composition or therapeutic agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or γ-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment. The treatment regime may indicate one or more of: the type of chemotherapy to administer to the patient;

the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs may be selected from: Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome) Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), [No Entries], Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VelP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib) and Zytiga (Abiraterone Acetate).

### Doses/Dosage Regimes

Multiple doses of the antigen-binding molecule, polypeptide, CAR, nucleic acid (or plurality thereof), expression vector (or plurality thereof), cell or composition may be provided. References to “antigen-binding molecule” in the following paragraphs also encompass one or more other articles according to the disclosure (polypeptide, CAR, nucleic acid (or plurality thereof), expression vector (or plurality thereof), cell or composition), and vice versa. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days: such as 4, 5, 6, 8, 9 or 10 days; 11, 12, 13, 15, 16 or 17 days; 18, 19, 20, 22, 23, or 24 days; or 25, 26, 27, 29, 30 or 31 days). That is, there may be one treatment event every 7 days, every 14 days, every 21 days, or every 28 days. There may be a treatment holiday between doses/administrations of about 7 days (plus or minus 3, 2, or 1 days), about 14 days (plus or minus 3, 2, or 1 days), about 21 days (plus or minus 3, 2, or 1 days), or about 28 days (plus or minus 3, 2, or 1 days), respectively.

In some aspects of the present disclosure, the antigen-binding molecule is administered once every week (e.g. once every 7 days plus or minus 3, 2, or 1 days). That is, there may be one treatment event/administration every week.

In some aspects of the present disclosure, the antigen-binding molecule is administered once every two weeks (e.g. once every 14 days plus or minus 3, 2, or 1 days). That is, there may be one treatment event/administration every two weeks.

In some aspects of the present disclosure, the antigen-binding molecule is administered once every three weeks (e.g. once every 21 days plus or minus 3, 2, or 1 days). That is, there may be one treatment event/administration every three weeks.

In some aspects of the present disclosure, the antigen-binding molecule is administered within one or more periods of three weeks/21 days (plus/minus 3, 2, or 1 days). Each period of three weeks/21 days may be referred to as one administration ‘cycle’, i.e. one cycle may be three weeks/21 days (plus/minus 3, 2, or 1 days). Administration may continue for 3, 6, 9, 12, 15, 18, 21, 24, 27, 30 or more weeks, up to 105 weeks. Administration may continue for 1, 2, 3, 4, 5, 6 or more cycles, up to 35 cycles (e.g. with each cycle comprising 21 days/three weeks). Administration may continue for 1, 2, 3, 4, 5, 6 or more months, up to 24 months (e.g. with each cycle comprising 21 days/three weeks). The cycles/periods of 21 days may be continuous/consecutive, or they may be separated by e.g. 1, 2, 3, 4, 5, 6 or 7 days, or 1, 2, 3, 4, 5, 6 or more weeks.

In some embodiments the antigen-binding molecule is administered one, two or three times during a period of three weeks/21 days (plus/minus 3, 2, or 1 days). That is, there may be one, two or three doses administered per three-week/21-day cycle.

In some embodiments the antigen-binding molecule is administered once a week for a period of three weeks. The antigen-binding molecule may be administered once every week for one, two, three, four, five, six or more cycles (that is, once every week for 1, 2, 3, 4, 5, 6 or more periods of 21 days, or once every week for 3, 6, 9, 12, 15, 18, 21, 24, 27, 30 or more weeks). In some embodiments the antigen binding molecule is administered on Days 1, 8 and 15 (plus/minus 3, 2, or 1 days) of the 21-day/three-week cycle. There may be a treatment holiday of ˜7 days (plus or minus 3, 2, or 1 days) between each dose.

In some embodiments the antigen-binding molecule is administered twice (two times) during a period of three weeks/21 days (plus/minus 3, 2, or 1 days). That is, there may be two doses administered per three-week/21-day cycle. In some embodiments the two doses are administered 7 days apart (plus/minus 3, 2, or 1 days), within the period of three weeks/21 days. The antigen-binding molecule may be administered twice every one, two, three, four, five, six or more cycles (that is, twice every 1, 2, 3, 4, 5, 6 or more periods of three weeks/21 days, or twice every three weeks/21 days for 3, 6, 9, 12, 15, 18, 21, 24, 27, 30 or more weeks). In some embodiments the antigen binding molecule is administered on Days 1 and 8 (plus/minus 3, 2, or 1 days) of each 21-day/three-week cycle. There may be a treatment holiday of ˜14 days (plus or minus 3, 2, or 1 days) between doses in different cycles (e.g. between a dose on Day 8 of cycle 1 and a dose on Day 1 of cycle 2).

In some embodiments the antigen-binding molecule is administered once (one time) during a period of three weeks/21 days (plus/minus 3, 2, or 1 days). That is, there may be one dose administered per three-week/21-day cycle. The antigen-binding molecule may be administered once every one, two, three, four, five, six or more cycles (that is, once every 1, 2, 3, 4, 5, 6 or more periods of three weeks/21 days, or once every three weeks/21 days for 3, 6, 9, 12, 15, 18, 21, 24, 27, 30 or more weeks). In some embodiments the antigen binding molecule is administered on Day 1 (plus/minus 3, 2, or 1 days) of each 21-day/three-week cycle. There may be a treatment holiday of ˜21 days (plus or minus 3, 2, or 1 days) between doses in different cycles (e.g. between a dose on Day 1 of cycle 1 and a dose on Day 1 of cycle 2).

In some embodiments the antigen-binding molecule is administered one, two, three, or four times during a period of four weeks/28 days (plus/minus 3, 2, or 1 days). That is, there may be one, two, three or four doses administered per four-week/28-day cycle (i.e. an administration cycle may be four weeks/28 days, plus/minus 3, 2, or 1 days). The antigen-binding molecule may be administered once, twice, three times or four times every one, two, three, four, five, six or more cycles (that is, once every 1, 2, 3, 4, 5, 6 or more periods of four weeks/28 days, or once, twice, three times or four times every four weeks/28 days for 4, 8, 12, 16, 20, 24, 28, 32 or more weeks). In some embodiments the antigen binding molecule is administered on one or more of Days 1, 8, 15 and/or 22 (each plus/minus 3, 2, or 1 days) of each 28-day/four-week cycle.

In some embodiments the antigen-binding molecule is administered twice (two times) during a period of four weeks/28 days (plus/minus 3, 2, or 1 days). That is, there may be two doses administered per four-week/28-day cycle. In some embodiments the two doses are administered 7 days apart or 14 days apart (plus/minus 3, 2, or 1 days), within the period of four weeks/28 days. The antigen-binding molecule may be administered twice every one, two, three, four, five, six or more cycles (that is, twice every 1, 2, 3, 4, 5, 6 or more periods of four weeks/28 days, or twice every four weeks/28 days for 4, 8, 12, 16, 20, 24, 28, 32, 36, 40 or more weeks). In some embodiments the antigen binding molecule is administered on Days 1 and 15 (plus/minus 3, 2, or 1 days) of each 28-day/four-week cycle. There may be a treatment holiday of ˜14 days (plus or minus 3, 2, or 1 days) between doses in the same cycle (e.g. between a dose on

Day 1 of cycle 1 and a dose on Day 51 of cycle 1). There may be a treatment holiday of ˜14 days (plus or minus 3, 2, or 1 days) between doses in different cycles (e.g. between a dose on Day 15 of cycle 1 and a dose on Day 1 of cycle 2).

In some embodiments, administration comprises administering at least 2 mg, at least 3, at least 5 mg, at least 7 mg, at least 10 mg, at least 11 mg, at least 12 mg, at least 13 mg, at least 14 mg, at least 15 mg, at least 16 mg, at least 17 mg, at least 18 mg, at least 19 mg, at least 20 mg, at least 21 mg, at least 22 mg, at least 23 mg, at least 24 mg, or at least 25 mg, e.g. per administration.

In some embodiments, administration (e.g. in the schedules above) comprises administering about 7 mg of the antigen-binding molecule, e.g. per administration. In some embodiments, administration (e.g. in the schedules above) comprises administering about 10 mg of the antigen-binding molecule, e.g. per administration. In some embodiments, administration (e.g. in the schedules above) comprises administering about 20 mg of the antigen-binding molecule, e.g. per administration. In some embodiments, administration (e.g. in the schedules above) comprises administering about 2.2 mg of the antigen-binding molecule, e.g. per administration. In some embodiments, administration (e.g. in the schedules above) comprises administering about 3.3 mg of the antigen-binding molecule, e.g. per administration. In some embodiments, administration (e.g. in the schedules above) comprises administering about 3.5 mg of the antigen-binding molecule, e.g. per administration.

In some embodiments, the antigen-binding molecule is administered at a dose from 3.5 mg to 1600 mg per administration or per administration cycle, e.g. as described herein. In some embodiments, the antigen-binding molecule is administered at a dose from 3.5 mg to 20 mg per administration or per administration cycle, e.g. as described herein. In some embodiments, the antigen-binding molecule is administered at a dose from 20 mg to 80 mg per administration or per administration cycle, e.g. as described herein. In some embodiments, the antigen-binding molecule is administered at a dose from 60 mg to 400 mg per administration or per administration cycle, e.g. as described herein. In some embodiments, the antigen-binding molecule is administered at a dose from 100 mg to 250 mg per administration or per administration cycle, e.g. as described herein. In some embodiments, the antigen-binding molecule is administered at a dose from 300 mg to 800 mg per administration or per administration cycle, e.g. as described herein. In some embodiments, the antigen-binding molecule is administered at a dose from 800 mg to 1800 mg per administration or per administration cycle, e.g. as described herein. In some embodiments, the antigen-binding molecule is administered at a dose from 1000 mg to 1600 mg per administration or per administration cycle, e.g. as described herein.

In some embodiments, administration (e.g. in the schedules above) comprises administering at least 3.5 mg, at least 5 mg, at least 7.5 mg, at least 10 mg, at least 12.5 mg, at least 15 mg, at least 17.5 mg, at least 20 mg, at least 22.5 mg, at least 25 mg, at least 27.5 mg, at least 30 mg, at least 32.5 mg, at least 35 mg, at least 37.5 mg, at least 40 mg, at least 42.5 mg, at least 45 mg, at least 47.5 mg, at least 50 mg, at least 52.5 mg, at least 55 mg, at least 57.5 mg, at least 60 mg, at least 62.5 mg, at least 65 mg, at least 67.5 mg, or at least 70 mg of antigen-binding molecule per administration or per administration cycle, e.g. per period of three weeks/21 days or four weeks/28 days.

In some embodiments, administration (e.g. in the schedules above) comprises administering at least 80 mg, at least 90 mg, at least 100 mg, at least 110 mg, at least 120 mg, at least 130 mg, at least 140 mg, at least 150 mg, at least 160 mg, at least 170 mg, at least 180 mg, at least 190 mg, at least 200 mg, at least 210 mg, at least 220 mg, at least 230 mg, at least 240 mg, at least 250 mg, at least 260 mg, at least 270 mg, at least 280 mg, at least 290 mg, at least 300 mg, at least 310 mg, at least 320 mg, at least 330 mg, at least 340 mg, at least 350 mg, at least 360 mg, at least 370 mg, at least 380 mg, at least 390 mg, at least 400 mg, at least 410 mg, at least 420 mg, at least 430 mg, at least 440 mg, at least 450 mg, at least 460 mg, at least 470 mg, at least 480 mg, at least 490 mg, at least 500 mg, at least 510 mg, at least 520 mg, at least 530 mg, at least 540 mg, at least 550 mg, at least 560 mg, at least 570 mg, at least 580 mg, at least 590 mg, at least 600 mg, at least 610 mg, at least 620 mg, at least 630 mg, at least 640 mg, at least 650 mg, at least 660 mg, at least 670 mg, at least 680 mg, at least 690 mg, at least 700 mg, at least 710 mg, at least 720 mg, at least 730 mg, at least 740 mg, at least 750 mg, at least 760 mg, at least 770 mg, at least 780 mg, at least 790 mg, at least 800 mg, at least 810 mg, at least 820 mg, at least 830 mg, at least 840 mg, at least 850 mg, at least 860 mg, at least 870 mg, at least 880 mg, at least 890 mg, at least 900 mg, at least 910 mg, at least 920 mg, at least 930 mg, at least 940 mg, at least 950 mg, at least 960 mg, at least 970 mg, at least 980 mg, at least 990 mg, at least 1000 mg, at least 1050 mg, at least 1100 mg, at least 1150 mg, at least 1200 mg, at least 1250 mg, at least 1300 mg, at least 1350 mg, at least 1400 mg, at least 1450 mg, at least 1500 mg, at least 1550 mg, at least 1600 mg, at least 1650 mg, at least 1700 mg, at least 1750 mg, at least 1800 mg, at least 1850 mg, at least 1900 mg, at least 1950 mg, or at least 2000 mg, of antigen-binding molecule per administration or per administration cycle, e.g. per period of three weeks/21 days or four weeks/28 days.

In some embodiments, administration (e.g. in the schedules above) comprises administering (up to or at least) 3.5 mg, 7 mg, 10.5 mg, 17.5 mg, 20 mg, 21 mg, 40 mg, 60 mg, 72 mg, 120 mg, 180 mg, 240 mg, 360 mg, 400 mg, 800 mg, 1200 mg, 1600 mg, 1900 mg, 2200 mg, or 2500 mg of antigen-binding molecule per administration or per administration cycle, e.g. per period of three weeks/21 days or four weeks/28 days.

In some embodiments, administration (e.g. in the schedules above) comprises administering 60-90 mg, 90-120 mg, 120-180 mg, 180-240 mg or 240-360 mg of antigen-binding molecule per administration or per administration cycle.

In some embodiments, administration (e.g. in the schedules above) comprises administering about 0.05 mg/kg, about 0.1 mg/kg, about 0.15 mg/kg, about 0.25 mg/kg, about 0.3 mg/kg, about 0.5 mg/kg, about 0.9 mg/kg, about 1 mg/kg, about 1.5 mg/kg, about 2 mg/kg, about 2.5 mg/kg, about 3 mg/kg, about 5 mg/kg, about 7.5 mg/kg, about 10 mg/kg, about 12.5 mg/kg, about 15 mg/kg, about 17.5 mg/kg, about 20 mg/kg, about 22.5 mg/kg or about 25 mg/kg (or at least one of the listed doses) of antigen-binding molecule per administration or per administration cycle, e.g. per period of three weeks/21 days or four weeks/28 days.

In some embodiments, administration (e.g. in the schedules above) comprises administering up to 10.5 mg, up to 21 mg, up to 31.5 mg, up to 52.5 mg, up to 60 mg, up to 63 mg, up to 120 mg, up to 216 mg, up to 180 mg, up to 360 mg, up to 540 mg, up to 720 mg, up to 1080 mg, up to 1200 mg, up to 2400 mg, up to 3600 mg, up to 4800 mg, up to 5000 mg, up to 5500 mg, up to 5700 mg, up to 6000 mg, up to 6600 mg or up to 7500 mg of antigen-binding molecule per administration cycle, e.g. per period of three weeks/21 days or four weeks/28 days. In some embodiments, administration (e.g. in the schedules above) comprises administering 10.5 mg to 7500 mg of antigen-binding molecule per administration cycle, e.g. per period of three weeks/21 days or four weeks/28 days. In some embodiments, administration (e.g. in the schedules above) comprises administering 10.5 mg to 31.5 mg, 31.5 mg to 66 mg, 66 mg to 120 mg, 120 mg to 216 mg, 216 mg to 360 mg, 360 mg to 720 mg, 720 mg to 1080 mg, 1080 mg to 1200 mg, 1200 mg to 2400 mg, 2400 mg to 3600 mg, 3600 mg to 4800 mg, 4800 mg to 6000 mg, or 6000 mg to 7500 mg of antigen-binding molecule per administration cycle, e.g. per period of three weeks/21 days.

In some embodiments, administration (e.g. in the schedules above) comprises administering up to 14 mg, up to 28 mg, up to 42 mg, up to 70 mg, up to 80 mg, up to 84 mg, up to 160 mg, up to 240 mg, up to 288 mg, up to 480 mg, up to 720 mg, up to 960 mg, up to 1440 mg, up to 1600 mg, up to 3200 mg, up to 4800 mg, up to 6400 mg, up to 6600 mg, up to 7200 mg, up to 7600 mg, up to 8000 mg, up to 8800 mg or up to 10000 mg of antigen-binding molecule per administration cycle, e.g. per period of four weeks/28 days. In some embodiments, administration (e.g. in the schedules above) comprises administering 14 mg to 1000 mg of antigen-binding molecule per administration cycle, e.g. per period of four weeks/28 days. In some embodiments, administration (e.g. in the schedules above) comprises administering 14 mg to 42 mg, 42 mg to 80 mg, 80 mg to 120 mg, 120 mg to 160 mg, 160 mg to 240 mg, 240 mg to 480 mg, 480 mg, to 720 mg, 720 mg to 960 mg, 960 mg to 1600 mg, 1600 mg to 3200 mg, 3200 mg to 4800 mg, 4800 mg to 6400 mg, 6400 mg to 7200 mg, 7200 mg to 8000 mg, or 8000 to 10000 mg of antigen-binding molecule per administration cycle, e.g. per period of four weeks/28 days.

The antigen-binding molecule, polypeptide, CAR, nucleic acid (or plurality thereof), expression vector (or plurality thereof), cell or composition is administered at one or more doses or administration schedules described herein in combination with a second therapeutic agent. The second agent may be capable of signalling mediated by PD-1. The agent capable of inhibiting signalling mediated by PD-1 may be a PD-1- or PD-L1-targeted agent. The agent capable of inhibiting signalling mediated by PD-1 may e.g. be an antibody capable of binding to PD-1 or PD-L1 and inhibiting PD-1-mediated signalling. In some embodiments the agent is an antagonist anti-PD-1 antibody. In some embodiments the agent is an antagonist anti-PD-L1 antibody. The agent may be Pembrolizumab (Keytruda; MK-3475, lambrolizumab), Nivolumab (Opdivo), Cemiplimab (Libtayo), Atezolizumab (Tecentriq), Avelumab (Bavencio), or Durvalumab (Imfinzi). Doses and administration regimes of such antibodies are available in the art.

In some embodiments, the second therapeutic agent e.g. an agent capable of inhibiting signalling mediated by PD-1, is administered at a dose of about 200 mg once (one time) during a period of three weeks/21 days (plus/minus 3, 2, or 1 days).

As used herein, “about” refers to the specified dose, and plus or minus 10% of that dose. For example, a dose of “about 3.5 mg” refers to a dose ranging from 3.15 mg to 3.85 mg, including a dose of 3.5 mg.

In some embodiments, the antigen-binding molecule is administered as a 60-minute IV infusion (−5 min/+10 min). In some embodiments, the antigen-binding molecule is administered as a 30-minute IV infusion (−5 min/+10 min). In some embodiments, the antigen-binding molecule is administered as a 45-minute IV infusion (−5 min/+10 min). The antigen-binding molecule may be formulated for IV infusion, e.g. in a composition according to the present disclosure.

The antigen-binding molecule may be co-administered with appropriate supportive care agents, e.g. hematopoietic growth factors, corticosteroids, anti-depressants, diphenhydramine, acetaminophen, thrombopoietic agents (i.e., romiplostim, eltrombopag) or granulocyte colony stimulating factor (G-CSF) agents (i.e., filgrastim, peg filgrastim), anti-emetic and anti-diarrheal agents, appetite stimulants, stimulants (i.e., modafinil), anti-cachexia therapy (i.e., fish-oil supplements), anti-depressants, opiate and non-opiate analgesics, antibiotics, selective use of corticosteroids, platelet/neutrophil growth factors, bisphosphonate therapy, anti-receptor activator of nuclear factor kappa β ligand therapy (e.g., denosumab), gonadotrophin releasing hormone (GnRH) agonists and/or LHRH agonists.

The antigen-binding molecule may be co-administered with one or more agents to treat or prevent cytokine release syndrome, e.g. corticosteroids, anti-IL-6 therapy (e.g. tocilizumab), nonsteroidal anti-inflammatory agents, epinephrine, IV fluids, antihistamines, NSAIDs, acetaminophen, narcotics, oxygen, and/or vasopressors.

### Methods of Detection

The invention also provides the articles of the present invention for use in methods for detecting, localizing or imaging VISTA, or cells expressing VISTA (e.g. MDSCs). The antigen-binding molecules described herein may be used in methods that involve the antigen-binding molecule to VISTA. Such methods may involve detection of the bound complex of the antigen-binding molecule and VISTA.

In particular, detection of VISTA may be useful in methods of diagnosing/prognosing a disease/condition in which cells expressing VISTA (e.g. MDSCs) are pathologically implicated, identifying subjects at risk of developing such diseases/conditions, and/or may be useful in methods of predicting a subject's response to a therapeutic intervention.

As such, a method is provided, comprising contacting a sample containing, or suspected to contain, VISTA with an antigen-binding molecule as described herein, and detecting the formation of a complex of the antigen-binding molecule and VISTA. Also provided is a method comprising contacting a sample containing, or suspected to contain, a cell expressing VISTA with an antigen-binding molecule as described herein and detecting the formation of a complex of the antigen-binding molecule and a cell expressing VISTA.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a tissue or tissues which are affected by the disease/condition (e.g. tissue or tissues in which symptoms of the disease manifest, or which are involved in the pathogenesis of the disease/condition).

Suitable method formats are well known in the art, including immunoassays such as sandwich assays, e.g. ELISA. The methods may involve labelling the antigen-binding molecule, or target(s), or both, with a detectable moiety, e.g. a fluorescent label, phosphorescent label, luminescent label, immuno-detectable label, radiolabel, chemical, nucleic acid or enzymatic label as described herein. Detection techniques are well known to those of skill in the art and can be selected to correspond with the labelling agent.

Methods of this kind may provide the basis of methods for the diagnostic and/or prognostic evaluation of a disease or condition, e.g. a cancer. Such methods may be performed in vitro on a patient sample, or following processing of a patient sample. Once the sample is collected, the patient is not required to be present for the in vitro method to be performed, and therefore the method may be one which is not practised on the human or animal body. In some embodiments the method is performed in vivo.

Detection in a sample may be used for the purpose of diagnosis of a disease/condition (e.g. a cancer), predisposition to a disease/condition, or for providing a prognosis (prognosticating) for a disease/condition, e.g. a disease/condition described herein. The diagnosis or prognosis may relate to an existing (previously diagnosed) disease/condition.

The present invention also provides methods for selecting/stratifying a subject for treatment with a VISTA-targeted agent. In some embodiments a subject is selected for treatment/prevention in accordance with the invention, or is identified as a subject which would benefit from such treatment/prevention, based on detection/quantification of VISTA, or cells expressing VISTA, e.g. in a sample obtained from the subject.

Such methods may involve detecting or quantifying VISTA and/or cells expressing VISTA (e.g. MDSCs), e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

Where an increased level of VISTA is detected, or where the presence of—or an increased number/proportion of—cells expressing VISTA (e.g. MDSCs) is detected in a sample obtained from a subject, the subject may be diagnosed as having a disease/condition in which MDSCs are pathologically implicated, or being at risk of developing such a disease/condition. In such methods, an “increased” level of expression or number/proportion of cells refers to a level/number/proportion which is greater than the level/number/proportion determined for an appropriate control condition, such as the level/number/proportion detected in a comparable sample (e.g. a sample of the same kind, e.g. obtained from the same fluid, tissue, organ etc.), e.g. obtained from a healthy subject.

Where an increased level of VISTA is detected, or where the presence of—or an increased number/proportion of—cells expressing VISTA (e.g. MDSCs) is detected in a sample obtained from a subject, the subject may be determined to have a poorer prognosis as compared to a subject determined to have a lower level of VISTA, or a reduced number/proportion of cells expressing VISTA (e.g. MDSCs) in a comparable sample (e.g. a sample of the same kind, e.g. obtained from the same fluid, tissue, organ etc.).

The antigen-binding molecules of the present invention are also useful in methods for predicting response to immunotherapy. “Immunotherapy” generally refers to therapeutic intervention aimed at harnessing the immune system to treat a disease/condition. Immunotherapy includes therapeutic intervention to increase the number/proportion/activity of effector immune cells (e.g. effector T cells (e.g. antigen-specific T cells, CAR-T cells), NK cells) in a subject. Immunotherapy to increase the number/proportion/activity of effector immune cells includes intervention to promote proliferation and/or survival of effector immune cells, inhibit signalling mediated by immune checkpoint molecules, promote signalling mediated by costimulatory receptors, enhance antigen presentation by antigen-presenting cells, etc. Immunotherapy to increase the number/proportion/activity of effector immune cells also encompasses intervention to increase the frequency of effector immune cells having a desired specificity or activity in a subject e.g. through adoptive cell transfer (ACT). ACT generally involves obtaining immune cells from a subject, typically by drawing a blood sample from which immune cells are isolated. The cells are then typically treated or altered in some way, and then administered either to the same subject or to a different subject. ACT is typically aimed at providing an immune cell population with certain desired characteristics to a subject, or increasing the frequency immune cells with such characteristics in that subject. In some embodiments

ACT may e.g. be of cells comprising a chimeric antigen receptor (CAR) specific for a target antigen or cell type of interest. Immunotherapy also includes therapeutic intervention to decrease the number/proportion/activity of suppressor immune cells (e.g. regulatory T cells, MDSCs) in a subject. Immunotherapy to decrease the number/proportion/activity of suppressor immune cells includes intervention to cause or potentiate cell killing of suppressor immune cells, and inhibit signalling mediated by immune checkpoint molecules.

Where an increased level of VISTA is detected, or where the presence of—or an increased number/proportion of—cells expressing VISTA (e.g. MDSCs) is detected in a sample obtained from a subject, the subject may be predicted to have a poorer response to immunotherapy to increase the number/proportion/activity of effector immune cells in the subject as compared to a subject determined to have a lower level of VISTA, or a reduced number/proportion of cells expressing VISTA (e.g. MDSCs) in a comparable sample (e.g. a sample of the same kind, e.g. obtained from the same fluid, tissue, organ etc.). Where an increased level of VISTA is detected, or where the presence of—or an increased number/proportion of—cells expressing VISTA (e.g. MDSCs) is detected in a sample obtained from a subject, the subject may be predicted to have an improved response to immunotherapy aimed at reducing the number/proportion/activity of suppressor immune cells in the subject as compared to a subject determined to have a lower level of VISTA, or a reduced number/proportion of cells expressing VISTA (e.g. MDSCs) in a comparable sample (e.g. a sample of the same kind, e.g. obtained from the same fluid, tissue, organ etc.).

In some embodiments the methods comprise determining the relative size/activity of suppressor immune cell compartment and the effector immune cell compartment. For example, in some embodiments the methods employ the antigen-binding molecules described herein in methods for determining the ratio of VISTA-expressing cells (e.g. MDSCs, TAMs, neutrophils) to effector immune cells. A subject having an increased ratio may be predicted to have an improved response to immunotherapy aimed at reducing the number/proportion/activity of suppressor immune cells, and/or may be predicted to have a poorer response to immunotherapy to increase the number/proportion/activity of effector immune cells as compared to a subject determined to have a lower ratio.

The diagnostic and prognostic methods of the present invention may be performed on samples obtained from a subject at multiple time points throughout the course of the disease and/or treatment, and may be used monitor development of the disease/condition over time, e.g. in response to treatment administered to the subject. The results of characterisation in accordance with the methods may be used to inform clinical decisions as to when and what kind of therapy to administer to a subject.

Methods of diagnosis or prognosis may be performed in vitro on a sample obtained from a subject, or following processing of a sample obtained from a subject. Once the sample is collected, the patient is not required to be present for the in vitro method of diagnosis or prognosis to be performed and therefore the method may be one which is not practised on the human or animal body.

### Subjects

The subject in accordance with aspects the invention described herein may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer), may be suspected of having such a disease/condition, or may be at risk of developing/contracting such a disease/condition.

In embodiments according to the present invention the subject is preferably a human subject. In some embodiments, the subject to be treated according to a therapeutic or prophylactic method of the invention herein is a subject having, or at risk of developing, a cancer. In embodiments according to the present invention, a subject may be selected for treatment according to the methods based on characterisation for certain markers of such disease/condition.

Subjects according to the present disclosure may comprise cells, e.g. immune cells and/or tumour cells, that express or over-express VISTA/an interaction partner for VISTA, e.g. as described herein.

Subjects may comprise advanced or metastatic cancer, such as solid tumours, e.g. confirmed histologically (e.g via immunohistochemistry on tumour biopsy). The cancer may be resistant or refractory to conventional treatment, or no conventional therapy may exist. The subject may have a cancer that is resistant to immune checkpoint targeted therapies, e.g. anti-CTLA4 and/or anti-PD-1/PD-L1 therapies.

The subject may have triple negative breast cancer (TBNC), e.g. advanced, metastatic, resistant or refractory TBNC, and may have received prior treatment for TNBC, e.g. with little or no therapeutic benefit or demonstrating disease progression during/after treatment.

The subject may have non-small cell lung cancer (NSCLC), e.g. advanced, metastatic, resistant or refractory TBNC, and may have already received prior treatment for NSCLC, e.g. with little or no therapeutic benefit or demonstrating disease progression during/after treatment. The subject may comprise ROS rearrangements, BRAF V600E mutation, and/or met exon 14 skipping mutation. In some embodiments, the subject comprises cells without an activating mutation of the epidermal growth factor receptor (EGFR) or anaplastic lymphoma kinase (ALK).

The subject may have received prior treatment with an anti-PD-1 or anti-PD-L1 therapy, e.g. anti-PD-1 or anti-PD-L1 antibody. The subject may have received prior treatment with atezolizumab, e.g. in combination with chemotherapy, e.g. with little or no therapeutic benefit or demonstrating disease progression during/after treatment.

In some embodiments, a subject may be selected for treatment described herein based on the detection of cells expressing/overexpressing VISTA and/or an interaction partner for VISTA, or detection of a cancer/tumor comprising cells expressing/overexpressing VISTA and/or an interaction partner for VISTA, e.g. in a sample obtained from the subject. The cancer or cells may be described as VISTA IHC+. Methods according to the present disclosure may comprise detecting a cancer or tumour comprising cells expressing/overexpressing VISTA and/or an interaction partner for VISTA in a subject, e.g. as described herein, e.g. in a sample obtained from the subject.

In some embodiments, a subject may be selected for treatment described herein based on the detection of cells expressing/overexpressing PD-1 and/or PD-L1, e.g. in a sample obtained from the subject. The cancer or cells may be described as PD-1 and/or PD-L1 IHC+.

Methods according to the present disclosure may comprise detecting biomarkers, e.g. circulating tumour markers including cell free DNA (cfDNA) alteration allele fraction/tumour fraction (e.g. tumour-derived cfDNA such as ctDNA). Methods according to the present invention may comprise detecting cytokines, e.g. IL-2, IL-6, IL-8 and/or TNFα. Methods according to the present invention may comprise proteomic analyses on immune and cancer cells, e.g. for VISTA and/or PD-1/PD-L1 expression. Methods may comprise detecting the presence and/or changes of tumour-infiltrating leukocytes, immune-related mRNA expression signatures, and VISTA and PD-1/PD-L1 expression.

One or more tumour serum markers (or panels of markers) may be evaluated before, during and after the subject receives treatment, as appropriate for the subject's cancer/tumour type, including but not limited to CA-125, HE4 and OVA1 in ovarian cancer; CEA in colorectal cancer; CA19-9 in pancreatic and gastric cancer; PSA, PAP, PCA3, the Oncotype DX GPS signature and the Prolaris signature in prostate cancer; BTA, FGFR2 and/or FGFR3 gene mutations, NMP22, and chromosomes 3, 7, 17, and 9p21 in bladder cancer; ALK gene rearrangements and overexpression, EGFR gene mutation, NSE, PD-L1 and ROS1 gene rearrangement in NSCLC; BRCA1 and/or BRCA2 in ovarian and breast cancers; BRAF V600 (e.g. BRAF V600E/) and KRAS mutations in e.g. colorectal cancer and NSCLC; CA15-3/CA27.29, ER/PR, uPA, PAI-1 and the Mammaprint signature in breast cancer; cytokeratin fragment 21-1 in lung cancer; DCP in HCC, DPD mutation in breast, colorectal, gastric and pancreatic cancers; thyroglobulin in thyroid cancer; and NTRK gene fusion in any solid tumour.

Any biomarker described herein may be detected before, during and/or after treatment using the methods disclosed herein, e.g. to select a suitable subject for treatment and/or to monitor the course or success of the treatment. Detection of a biomarker after treatment may be compared with detection of that biomarker before treatment.

### Kits

In some aspects of the invention described herein a kit of parts is provided. In some embodiments the kit may have at least one container having a predetermined quantity of an antigen-binding molecule, polypeptide, CAR, nucleic acid (or plurality thereof), expression vector (or plurality thereof), cell or composition described herein.

In some embodiments the kit comprises a 50 mg/mL solution of antigen-binding molecule, e.g. in a composition according to the present disclosure. In some embodiments the kit comprises 50 mg/ml antigen binding molecule in a composition comprising 20 mM histidine, 8% (w/v) sucrose, 0.02% (w/v) polysorbate 80 at pH 5.5.

The composition comprising 50 mg/mL antigen-binding molecule may be diluted before use. The kit may comprise instructions for dilution. The kit may comprise 5% dextrose for use in diluting the composition, e.g. to arrive at a composition suitable for intravenous administration, e.g. as described herein. The kit may comprise a composition comprising antigen-binding molecule at a concentration of at least 0.07 mg/mL, or another concentration according to the present disclosure, e.g. provided as is, or after dilution with dextrose.

The antigen-binding molecule, or other article of the disclosure, may be lyophilised (i.e. the container may comprise lyophilised antigen-binding molecule or other article). The lyophilised agent may be reconstituted in a composition buffer, e.g. according to the present disclosure. The kit may comprise instructions for reconstituting the antigen-binding molecule/other article.

The container may be any suitable container, e.g. a glass vial.

In some embodiments, the kit may comprise materials for producing an antigen-binding molecule, polypeptide, CAR, nucleic acid (or plurality thereof), expression vector (or plurality thereof), cell or composition described herein.

The kit may provide the antigen-binding molecule, polypeptide, CAR, nucleic acid (or plurality thereof), expression vector (or plurality thereof), cell or composition together with instructions for administration to a patient in order to treat a specified disease/condition, e.g. using a dose/dosing regime as described herein and/or to treat a disease/condition as described herein.

In some embodiments the kit may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the specific disease or condition. The therapeutic agent may also be formulated so as to be suitable for injection or infusion to a tumor or to the blood. The therapeutic agent may be any such agent described herein. The therapeutic agent may be an anti-PD-1/anti-PD-L1 agent, e.g. antibody. The therapeutic agent may be an antagonist anti-PD-1 antibody or an antagonist anti-PD-L1 antibody.

### Sequence Identity

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

Sequences

| SEQ | | |
|---|---|---|
| ID | | |
| NO: | DESCRIPTION | SEQUENCE |
| 1 | Human VISTA | MGVPTALEAGSWRWGSLLFALFLAASLGPVAAFKVATPYSLYVCPEGQNVTLTCRLLGPVDKGH |
| | (Q9H7M9-1, v3) | DVTFYKTWYRSSRGEVQTCSERRPIRNLTFQDLHLHHGGHQAANTSHDLAQRHGLESASDHHG |
| | | NFSITMRNLTLLDSGLYCCLVVEIRHHHSEHRVHGAMELQVQTGKDAPSNCVVYPSSSQDSENIT |
| | | AAALATGACIVGILCLPLILLLVYKQRQAASNRRAQELVRMDSNIQGIENPGFEASPPAQGIPEAKV |
| | | RHPLSYVAQRQPSESGRHLLSEPSTPLSPPGPGDVFFPSLDPVPDSPNFEVI |
| 2 | Mature human | FKVATPYSLYVCPEGQNVTLTCRLLGPVDKGHDVTFYKTWYRSSRGEVQTCSERRPIRNLTFQDL |
| | VISTA (Q9H7M9-1, | HLHHGGHQAANTSHDLAQRHGLESASDHHGNFSITMRNLTLLDSGLYCCLVVEIRHHHSEHRVH |
| | v3 positions 33 | GAMELQVQTGKDAPSNCVVYPSSSQDSENITAAALATGACIVGILCLPLILLLVYKQRQAASNRRA |
| | to 311) | QELVRMDSNIQGIENPGFEASPPAQGIPEAKVRHPLSYVAQRQPSESGRHLLSEPSTPLSPPGPG |
| | | DVFFPSLDPVPDSPNFEVI |
| 3 | Extracellular | FKVATPYSLYVCPEGQNVTLTCRLLGPVDKGHDVTFYKTWYRSSRGEVQTCSERRPIRNLTFQDL |
| | domain human | HLHHGGHQAANTSHDLAQRHGLESASDHHGNFSITMRNLTLLDSGLYCCLVVEIRHHHSEHRVH |
| | VISTA (Q9H7M9-1, | GAMELQVQTGKDAPSNCVVYPSSSQDSENITAA |
| | v3 positions 33 | |
| | to1 94) | |
| 4 | Transmembrane | ALATGACIVGILCLPLILLLV |
| | domain human | |
| | VISTA (Q9H7M9-1, | |
| | v3 positions 195 | |
| | to 215) | |
| 5 | Cytoplasmic domain | YKQRQAASNRRAQELVRMDSNIQGIENPGFEASPPAQGIPEAKVRHPLSYVAQRQPSESGRHLL |
| | human VISTA | SEPSTPLSPPGPGDVFFPSLDPVPDSPNFEVI |
| | (Q9H7M9-1, v3 | |
| | positions 216 to | |
| | 311) | |
| 6 | Ig-like V-type | FKVATPYSLYVCPEGQNVTLTCRLLGPVDKGHDVTFYKTWYRSSRGEVQTCSERRPIRNLTFQDL |
| | domain human | HLHHGGHQAANTSHDLAQRHGLESASDHHGNFSITMRNLTLLDSGLYCCLVVEIRHHHSEHRVH |
| | VISTA (Q9H7M9-1, | GAMELQV |
| | v3 positions 33 | |
| | to 168) | |
| 7 | Human VSIG-3 | MTSQRSPLAPLLLLSLHGVAASLEVSESPGSIQVARGQPAVLPCTFTTSAALINLNVIWMVTPLSNA |
| | isoform 1 | NQPEQVILYQGGQMFDGAPRFHGRVGFTGTMPATNVSIFINNTQLSDTGTYQCLVNNLPDIGGRN |
| | (Q5DX21-1, v3) | IGVTGLTVLVPPSAPHCQIQGSQDIGSDVILLCSSEEGIPRPTYLWEKLDNTLKLPPTATQDQVQGT |
| | | VTIRNISALSSGLYQCVASNAIGTSTCLLDLQVISPQPRNIGLIAGAIGTGAVIIIFCIALILGAFFY |
| | | WRSKNKEEEEEEIPNEIREDDLPPKCSSAKAFHTEISSSDNNTLTSSNAYNSRYWSNNPKVHRNTESV |
| | | SHFSDLGQSFSFHSGNANIPSIYANGTHLVPGQHKTLVVTANRGSSPQVMSRSNGSVSRKPRPPH |
| | | THSYTISHATLERIGAVPVMVPAQSRAGSLV |
| 8 | Human VSIG-3 | MSLVELLLWWNCFSRTGVAASLEVSESPGSIQVARGQPAVLPCTFTTSAALINLNVIWMVTPLSNA |
| | isoform 2 | NQPEQVILYQGGQMFDGAPRFHGRVGFTGTMPATNVSIFINNTQLSDTGTYQCLVNNLPDIGGRN |
| | (Q5DX21-2) | IGVTGLTVLVPPSAPHCQIQGSQDIGSDVILLCSSEEGIPRPTYLWEKLDNTLKLPPTATQDQVQGT |
| | | VTIRNISALSSGLYQCVASNAIGTSTCLLDLQVISPQPRNIGLIAGAIGTGAVIIIFCIALILGAFFY |
| | | WRSKNKEEEEEEIPNEIREDDLPPKCSSAKAFHTEISSSDNNTLTSSNAYNSRYWSNNPKVHRNTESV |
| | | SHFSDLGQSFSFHSGNANIPSIYANGTHLVPGQHKTLVVTANRGSSPQVMSRSNGSVSRKPRPPH |
| | | THSYTISHATLERIGAVPVMVPAQSRAGSLV |
| 9 | Human VSIG-3 | MSLVELLLWWNCFSRTGVAASLEVSESPGSIQVARGQPAVLPCTFTTSAALINLNVIWMVTPLSNA |
| | isoform 3 | NQPEQVILYQGGQMFDGAPRFHGRVGFTGTMPATNVSIFINNTQLSDTGTYQCLVNNLPDIGGRN |
| | (Q5DX21-3) | IGVTGLTVLVPPSAPHCQIQGSQDIGSDVILLCSSEEGIPRPTYLWEKLDNTLKLPPTATQDQVQGT |
| | | VTIRNISALSSAQPRNIGLIAGAIGTGAVIIIFCIALILGAFFYWRSKNKEEEEEEIPNEIREDDLPP |
| | | KCSSAKAFHTEISSSDNNTLTSSNAYNSRYWSNNPKVHRNTESVSHFSDLGQSFSFHSGNANIPSIYA |
| | | NGTHLVPGQHKTLVVTANRGSSPQVMSRSNGSVSRKPRPPHTHSYTISHATLERIGAVPVMVPA |
| | | QSRAGSLV |
| 10 | Mature human | LEVSESPGSIQVARGQPAVLPCTFTTSAALINLNVIWMVTPLSNANQPEQVILYQGGQMFDGAPRF |
| | VSIG-3 isoform 1 | HGRVGFTGTMPATNVSIFINNTQLSDTGTYQCLVNNLPDIGGRNIGVTGLTVLVPPSAPHCQIQGS |
| | (Q5DX21-1, v3 | QDIGSDVILLCSSEEGIPRPTYLWEKLDNTLKLPPTATQDQVQGTVTIRNISALSSGLYQCVASNAI |
| | positions 23 to | GTSTCLLDLQVISPQPRNIGLIAGAIGTGAVIIIFCIALILGAFFYWRSKNKEEEEEEIPNEIREDDL |
| | 431) | PPKCSSAKAFHTEISSSDNNTLTSSNAYNSRYWSNNPKVHRNTESVSHFSDLGQSFSFHSGNANIPS |
| | | IYANGTHLVPGQHKTLVVTANRGSSPQVMSRSNGSVSRKPRPPHTHSYTISHATLERIGAVPVMV |
| | | PAQSRAGSLV |
| 11 | Mature human | LEVSESPGSIQVARGQPAVLPCTFTTSAALINLNVIWMVTPLSNANQPEQVILYQGGQMFDGAPRF |
| | VSIG-3 isoform 2 | HGRVGFTGTMPATNVSIFINNTQLSDTGTYQCLVNNLPDIGGRNIGVTGLTVLVPPSAPHCQIQGS |
| | (Q5DX21-2 | QDIGSDVILLCSSEEGIPRPTYLWEKLDNTLKLPPTATQDQVQGTVTIRNISALSSGLYQCVASNAI |
| | positions 23 to | GTSTCLLDLQVISPQPRNIGLIAGAIGTGAVIIIFCIALILGAFFYWRSKNKEEEEEEIPNEIREDDL |
| | 431) | PPKCSSAKAFHTEISSSDNNTLTSSNAYNSRYWSNNPKVHRNTESVSHFSDLGQSFSFHSGNANIPS |
| | | IYANGTHLVPGQHKTLVVTANRGSSPQVMSRSNGSVSRKPRPPHTHSYTISHATLERIGAVPVMV |
| | | PAQSRAGSLV |
| 12 | Mature human | LEVSESPGSIQVARGQPAVLPCTFTTSAALINLNVIWMVTPLSNANQPEQVILYQGGQMFDGAPRF |
| | VSIG-3 isoform 3 | HGRVGFTGTMPATNVSIFINNTQLSDTGTYQCLVNNLPDIGGRNIGVTGLTVLVPPSAPHCQIQGS |
| | (Q5DX21-3 | QDIGSDVILLCSSEEGIPRPTYLWEKLDNTLKLPPTATQDQVQGTVTIRNISALSSAQPRNIGLIAGA |
| | positions 23 to | IGTGAVIIIFCIALILGAFFYWRSKNKEEEEEEIPNEIREDDLPPKCSSAKAFHTEISSSDNNTLTSS |
| | 407) | NAYNSRYWSNNPKVHRNTESVSHFSDLGQSFSFHSGNANIPSIYANGTHLVPGQHKTLVVTANRGS |
| | | SPQVMSRSNGSVSRKPRPPHTHSYTISHATLERIGAVPVMVPAQSRAGSLV |
| 13 | Extracellular | LEVSESPGSIQVARGQPAVLPCTFTTSAALINLNVIWMVTPLSNANQPEQVILYQGGQMFDGAPRF |
| | domain human | HGRVGFTGTMPATNVSIFINNTQLSDTGTYQCLVNNLPDIGGRNIGVTGLTVLVPPSAPHCQIQGS |
| | VSIG-3 isoforms 1 | QDIGSDVILLCSSEEGIPRPTYLWEKLDNTLKLPPTATQDQVQGTVTIRNISALSSGLYQCVASNAI |
| | and 2 | GTSTCLLDLQVISPQPRNIG |
| 14 | Extracellular | LEVSESPGSIQVARGQPAVLPCTFTTSAALINLNVIWMVTPLSNANQPEQVILYQGGQMFDGAPRF |
| | domain human | HGRVGFTGTMPATNVSIFINNTQLSDTGTYQCLVNNLPDIGGRNIGVTGLTVLVPPSAPHCQIQGS |
| | VSIG-3 isoform 3 | QDIGSDVILLCSSEEGIPRPTYLWEKLDNTLKLPPTATQDQVQGTVTIRNISALSSAQPRNIG |
| 15 | Transmembrane | LIAGAIGTGAVIIIFCIALIL |
| | domain human | |
| | VSIG-3 | |
| 16 | Cytoplasmic domain | GAFFYWRSKNKEEEEEEIPNEIREDDLPPKCSSAKAFHTEISSSDNNTLTSSNAYNSRYWSNNPK |
| | human VSIG-3 | VHRNTESVSHFSDLGQSFSFHSGNANIPSIYANGTHLVPGQHKTLVVTANRGSSPQVMSRSNGS |
| | | VSRKPRPPHTHSYTISHATLERIGAVPVMVPAQSRAGSLV |
| 17 | Ig-like V-type | LEVSESPGSIQVARGQPAVLPCTFTTSAALINLNVIWMVTPLSNANQPEQVILYQGGQMFDGAPRF |
| | domain human | HGRVGFTGTMPATNVSIFINNTQLSDTGTYQCLVNNLPDIGGRNIGVT |
| | VSIG-3 | |
| 18 | Ig-like C2-type | PSAPHCQIQGSQDIGSDVILLCSSEEGIPRPTYLWEKLDNTLKLPPTATQDQVQGTVTIRNISALSS |
| | domain human | GLYQCVASNAIGTSTCLLDLQVIS |
| | VSIG-3 | |
| 19 | Human VSIG-8 | MRVGGAFHLLLVCLSPALLSAVRINGDGQEVLYLAEGDNVRLGCPYVLDPEDYGPNGLDIEWMQ |
| | (P0DPA2-1, v1) | VNSDPAHHRENVFLSYQDKRINHGSLPHLQQRVRFAASDPSQYDASINLMNLQVSDTATYECRV |
| | | KKTTMATRKVIVTVQARPAVPMCWTEGHMTYGNDVVLKCYASGGSQPLSYKWAKISGHHYPYR |
| | | AGSYTSQHSYHSELSYQESFHSSINQGLNNGDLVLKDISRADDGLYQCTVANNVGYSVCVVEVKV |
| | | SDSRRIGVIIGIVLGSLLALGCLAVGIWGLVCCCCGGSGAGGARGAFGYGNGGGVGGGACGDLA |
| | | SEIREDAVAPGCKASGRGSRVTHLLGYPTQNVSRSLRRKYAPPPCGGPEDVALAPCTAAAACEA |
| | | GPSPVYVKVKSAEPADCAEGPVQCKNGLLV |
| 20 | Mature human | VRINGDGQEVLYLAEGDNVRLGCPYVLDPEDYGPNGLDIEWMQVNSDPAHHRENVFLSYQDKRI |
| | VSIG-8 (P0DPA2-1, | NHGSLPHLQQRVRFAASDPSQYDASINLMNLQVSDTATYECRVKKTTMATRKVIVTVQARPAVPM |
| | v1 positions 22 | CWTEGHMTYGNDVVLKCYASGGSQPLSYKWAKISGHHYPYRAGSYTSQHSYHSELSYQESFHS |
| | to 414) | SINQGLNNGDLVLKDISRADDGLYQCTVANNVGYSVCVVEVKVSDSRRIGVIIGIVLGSLLALGCLA |
| | | VGIWGLVCCCCGGSGAGGARGAFGYGNGGGVGGGACGDLASEIREDAVAPGCKASGRGSRVT |
| | | HLLGYPTQNVSRSLRRKYAPPPCGGPEDVALAPCTAAAACEAGPSPVYVKVKSAEPADCAEGPV |
| | | QCKNGLLV |
| 21 | Extracellular | VRINGDGQEVLYLAEGDNVRLGCPYVLDPEDYGPNGLDIEWMQVNSDPAHHRENVFLSYQDKRI |
| | domain human | NHGSLPHLQQRVRFAASDPSQYDASINLMNLQVSDTATYECRVKKTTMATRKVIVTVQARPAVPM |
| | VSIG-8 (P0DPA2-1, | CWTEGHMTYGNDVVLKCYASGGSQPLSYKWAKISGHHYPYRAGSYTSQHSYHSELSYQESFHS |
| | v1 positions 22 | SINQGLNNGDLVLKDISRADDGLYQCTVANNVGYSVCVVEVKVSDSRRIG |
| | to 263) | |
| 22 | Transmembrane | VIIGIVLGSLLALGCLAVGIW |
| | domain human | |
| | VSIG-8 (P0DPA2-1, | |
| | v1 positions 264 | |
| | to 284) | |
| 23 | Cytoplasmic domain | GLVCCCCGGSGAGGARGAFGYGNGGGVGGGACGDLASEIREDAVAPGCKASGRGSRVTHLLG |
| | human VSIG-8 | YPTQNVSRSLRRKYAPPPCGGPEDVALAPCTAAAACEAGPSPVYVKVKSAEPADCAEGPVQCKN |
| | (P0DPA2-1, v1 | GLLV |
| | positions 285 to | |
| | 414) | |
| 24 | Ig-like V-type | VRINGDGQEVLYLAEGDNVRLGCPYVLDPEDYGPNGLDIEWMQVNSDPAHHRENVFLSYQDKRI |
| | domain 1 human | NHGSLPHLQQRVRFAASDPSQYDASINLMNLQVSDTATYECRVKKTTMATRKVIVT |
| | VSIG-8 (P0DPA2-1, | |
| | v1 positions 22 | |
| | to 141) | |
| 25 | Ig-like V-type | PAVPMCWTEGHMTYGNDVVLKCYASGGSQPLSYKWAKISGHHYPYRAGSYTSQHSYHSELSYQ |
| | domain 2 human | ESFHSSINQGLNNGDLVLKDISRADDGLYQCTVANNVGYSVCVVEVKVS |
| | VSIG-8 (P0DPA2-1, | |
| | v1 positions 146 | |
| | to 257) | |
| 26 | Epitope recognised | SRGEVQTCSERRPI |
| | by anti-VISTA | |
| | antibody clones | |
| | 4M2-C12, 4M2-B4, | |
| | 4M2-C9, 4M2-D9, | |
| | 4M2-D5, 4M2-A8, | |
| | V4H1, V4H2, V4- | |
| | C1, V4-C9, V4-C24, | |
| | V4-C26, V4-C27, | |
| | V4-C28, V4-C30 | |
| | and V4-C31 | |
| 27 | Epitope recognised | FQDLHLHHGGHQAA |
| | by anti-VISTA | |
| | antibody clones | |
| | 2M1-B12 and 2M1- | |
| | D2 | |
| 28 | Epitope recognised | CLVVEIRHHHSEHR |
| | by anti-VISTA | |
| | antibody clones | |
| | 1M2-D2, 13D5p, | |
| | 13D5-1 and 13D5- | |
| | 13 | |
| 29 | Epitope recognised | DKGHDVTFYKT |
| | by anti-VISTA | |
| | antibody clone | |
| | 5M1-A11 | |
| 30 | Epitope recognised | RHHHSEHRVHG |
| | by anti-VISTA | |
| | antibody clone | |
| | 9M2-C12 | |
| 31 | Positions 61 to | DKGHDVTFYKTWYRSSRGEVQTCSERRPIRNLTFQDLHLHHGGHQAANTSHDLAQRHGLESAS |
| | 162 of human VISTA | DHHGNFSITMRNLTLLDSGLYCCLVVEIRHHHSEHRVHG |
| | (Q9H7M9-1, v3) | |
| 32 | 4M2-C12 heavy | EVKLVESGPGLVKPSQSLSLTCTVTGYSITSDYAWNWIRQFPGNKLEWMGYITYSGNISYNPSLR |
| | chain variable | SRISITRDTSKNQFFLQLNSVTPEDTATYSCARSLYYPWYFDVWGAGTTVTVSS |
| | region | |
| 33 | 4M2-C12, 4M2-B4, | GYSITSDYA |
| | V4H2, 4M2-D9, V4- | |
| | C1, V4-C9 heavy | |
| | chain CDR1 | |
| 34 | 4M2-C12, 4M2-B4, | ITYSGNI |
| | V4H1, V4H2 heavy | |
| | chain CDR2 | |
| 35 | 4M2-C12, 4M2-B4, | ARSLYYPWYFDV |
| | V4H1, V4H2 heavy | |
| | chain CDR3 | |
| 36 | 4M2-C12 heavy | EVKLVESGPGLVKPSQSLSLTCTVT |
| | chain FR1 | |
| 37 | 4M2-C12, 4M2-B4 | WNWIRQFPGNKLEWMGY |
| | heavy chain FR2 | |
| 38 | 4M2-C12, 4M2-B4 | SYNPSLRSRISITRDTSKNQFFLQLNSVTPEDTATYSC |
| | heavy chain FR3 | |
| 39 | 4M2-C12, 4M2-B4, | WGAGTTVTVSS |
| | V4H1, V4H2, | |
| | 13D5p, 13D5-1, | |
| | 13D5-13 heavy | |
| | chain FR4 | |
| 40 | 4M2-C12 light | DIVITQTPAILSTSPGEKVTMTCRASSSVGYIHWYQQKPGSSPKPWIYATSNLASGVPARFSGSGS |
| | chain variable | GTSNSLTITRVEAEDAATYYCQQWSSYPPITFGGGTKLEVK |
| | region | |
| 41 | 4M2-C12, 4M2-B4, | SSVGY |
| | V4H1, V4H2, V4- | |
| | C1, V4-C9, V4-C24, | |
| | V4-C26, V4-C27, | |
| | V4-C28, V4-C30, | |
| | V4-C31 light chain | |
| | CDR1 | |
| 42 | 4M2-C12, 4M2-B4, | ATS |
| | V4-C1, V4-C9, V4- | |
| | C28 light chain | |
| | CDR2 | |
| 43 | 4M2-C12, 4M2-B4, | QQWSSYPPIT |
| | V4H1, V4H2, V4-C1 | |
| | V4-C9, V4-C24, V4- | |
| | C26, V4-C27, V4- | |
| | C28, V4-C30, V4- | |
| | C31 light chain | |
| | CDR3 | |
| 44 | 4M2-C12 light | DIVITQTPAILSTSPGEKVTMTCRAS |
| | chain FR1 | |
| 45 | 4M2-C12, 4M2-B4 | IHWYQQKPGSSPKPWIY |
| | light chain FR2 | |
| 46 | 4M2-C12, 4M2-B4 | NLASGVPARFSGSGSGTSNSLTITRVEAEDAATYYC |
| | light chain FR3 | |
| 47 | 4M2-C12, 4M2-B4, | FGGGTKLEVK |
| | V4H1, V4H2, V4- | |
| | C1, V4-C9, V4-C24, | |
| | V4-C26, V4-C27, | |
| | V4-C28, V4-C30, | |
| | V4-C31 light chain | |
| | FR4 | |
| 48 | 4M2-B4 heavy | EVMLVESGPGLVKPSQSLSLTCTVTGYSITSDYAWNWIRQFPGNKLEWMGYITYSGNISYNPSLR |
| | chain variable | SRISITRDTSKNQFFLQLNSVTPEDTATYSCARSLYYPWYFDVWGAGTTVTVSS |
| | region | |
| 49 | 4M2-B4 heavy | EVMLVESGPGLVKPSQSLSLTCTVT |
| | chain FR1 | |
| 50 | 4M2-B4 light | DIVLTQTTAILSTSPGEKVTMTCRASSSVGYIHWYQQKPGSSPKPWIYATSNLASGVPARFSGSG |
| | chain variable | SGTSNSLTITRVEAEDAATYYCQQWSSYPPITFGGGTKLEVK |
| | region | |
| 51 | 4M2-B4 light | DIVLTQTTAILSTSPGEKVTMTCRA |
| | chain FR1 | |
| 52 | V4H1 heavy chain | EVQLLESGGGLVQPGGSLRLSCAASGYTITSDYAMSWVRQAPGKGLEWVSVITYSGNISYADSV |
| | variable region | KGRFTISRDNSKNTLYLQMNSLRAEDTAVYYCARSLYYPWYFDVWGAGTTVTVSS |
| 53 | V4H1 heavy chain | GYTITSDYA |
| | CDR1 | |
| 54 | V4H1 heavy chain | EVQLLESGGGLVQPGGSLRLSCAAS |
| | FR1 | |
| 55 | V4H1 heavy chain | MSWVRQAPGKGLEWVSV |
| | FR2 | |
| 56 | V4H1 heavy chain | SYADSVKGRFTISRDNSKNTLYLQMNSLRAEDTAVYYC |
| | FR3 | |
| 57 | V4H1 light chain | EIVITQSPATLSLSPGERATLSCRASSSVGYLAWYQQKPGQAPRPLIYDTSNRATGIPARFSGSGS |
| | variable region | GTDNTLTISSLEPEDFAVYYCQQWSSYPPITFGGGTKLEVK |
| 58 | V4H1 light chain | DTS |
| | CDR2 | |
| 59 | V4H1, V4-C1 light | EIVITQSPATLSLSPGERATLSCRAS |
| | chain FR1 | |
| 60 | V4H1 light chain | LAWYQQKPGQAPRPLIY |
| | FR2 | |
| 61 | V4H1 light chain | NRATGIPARFSGSGSGTDNTLTISSLEPEDFAVYYC |
| | FR3 | |
| 62 | V4H2 heavy chain | QVQLQESGPGLVKPSDTLSLTCTVSGYSITSDYAWSWIRQPPGKGLEWIGYITYSGNISYNPSLKS |
| | variable region | RVTISVDTSKNQFSLKLSSVTAADTAVYDCARSLYYPWYFDVWGAGTTVTVSS |
| 63 | V4H2, V4-C1, V4- | QVQLQESGPGLVKPSDTLSLTCTVS |
| | C9, V4-C24, V4- | |
| | C26, V4-C27, V4- | |
| | C28, V4-C30, V4- | |
| | C31 heavy chain | |
| | FR1 | |
| 64 | V4H2 heavy chain | WSWIRQPPGKGLEWIGY |
| | FR2 | |
| 65 | V4H2 heavy chain | SYNPSLKSRVTISVDTSKNQFSLKLSSVTAADTAVYDC |
| | FR3 | |
| 66 | V4H2 light chain | DIQMTQSPSSLSASVGDRVTITCRASSSVGYLNWYQQKPGKAPKPLIYAASSLQSGVPSRFSGSG |
| | variable region | SGTDNTLTISSLQPEDFATYYCQQWSSYPPITFGGGTKLEVK |
| 67 | V4H2, 4M2-D9 | AAS |
| | light chain CDR2 | |
| 68 | V4H2 light chain | DIQMTQSPSSLSASVGDRVTITCRA |
| | FR1 | |
| 69 | V4H2 light chain | LNWYQQKPGKAPKPLIY |
| | FR2 | |
| 70 | V4H2 light chain | SLQSGVPSRFSGSGSGTDNTLTISSLQPEDFATYYC |
| | FR3 | |
| 71 | 2M1-B12 heavy | EVQLQQSGAELVRPGTSVKTSCKASGYTFTNYWLGWVKERAGHGLEWIGEIFPGGGHTNYKEKF |
| | chain variable | KGKATLTADTSSSTAYMKLSSLTSEDSAVYFCAQIPLYYGHYRSAYWGQGTLVTVSA |
| | region | |
| 72 | 2M1-B12, 2M1-D2, | GYTFTNYW |
| | 13D5p, 13D5-1, | |
| | 13D5-13 heavy | |
| | chain CDR1 | |
| 73 | 2M1-B12, 2M1-D2 | IFPGGGHT |
| | heavy chain CDR2 | |
| 74 | 2M1-B12, 2M1-D2 | AQIPLYYGHYRSAY |
| | heavy chain CDR3 | |
| 75 | 2M1-B12 heavy | EVQLQQSGAELVRPGTSVKTSCKAS |
| | chain FR1 | |
| 76 | 2M1-B12, 2M1-D2 | LGWVKERAGHGLEWIGE |
| | heavy chain FR2 | |
| 77 | 2M1-B12, 2M1-D2 | NYKEKFKGKATLTADTSSSTAYMKLSSLTSEDSAVYFC |
| | heavy chain FR3 | |
| 78 | 2M1-B12, 2M1-D2, | WGQGTLVTVSA |
| | 1M2-D2 heavy | |
| | chain FR4 | |
| 79 | 2M1-B12 light | DIQMMQSPASLSASVGETVAITCGASENIYGALNWYQRKQGKSPQLLIYGATNLADGMSSRFSGS |
| | chain variable | GSGRQYSLKISSLHPDDVATYYCQNVLSTPYTFGGGTKLEIK |
| | region | |
| 80 | 2M1-B12, 2M1-D2 | ENIYGA |
| | light chain CDR1 | |
| 81 | 2M1-B12, 2M1-D2 | GAT |
| | light chain CDR2 | |
| 82 | 2M1-B12, 2M1-D2 | QNVLSTPYT |
| | light chain CDR3 | |
| 83 | 2M1-B12 light | DIQMMQSPASLSASVGETVAITCGAS |
| | chain FR1 | |
| 84 | 2M1-B12, 2M1-D2 | LNWYQRKQGKSPQLLIY |
| | light chain FR2 | |
| 85 | 2M1-B12, 2M1-D2 | NLADGMSSRFSGSGSGRQYSLKISSLHPDDVATYYC |
| | light chain FR3 | |
| 86 | 2M1-B12, 4M2-C9, | FGGGTKLEIK |
| | 2M1-D2, 4M2-D9, | |
| | 1M2-D2, 5M1-A11, | |
| | 4M2-D5, 4M2-A8, | |
| | 9M2-C12, 13D5p, | |
| | 13D5-1, 13d5-13 | |
| | light chain FR4 | |
| 87 | 4M2-C9 heavy | QVTLKECGPGILQPSQTLSLTCSFSGFSLSTSGMGVSWIRQPSGKGLEWLAHIYWDDDKRYNPSL |
| | chain variable | KSRLTISKDSSSNQVFLKITSVDTADTATYYCARRLDGYNDPYYFDYWGQGTTLTVSS |
| | region | |
| 88 | 4M2-C9, 5M1-A11 | GFSLSTSGMG |
| | heavy chain CDR1 | |
| 89 | 4M2-C9, 5M1-A11 | IYWDDDK |
| | heavy chain CDR2 | |
| 90 | 4M2-C9, 5M1-A11 | ARRLDGYNDPYYFDY |
| | heavy chain CDR3 | |
| 91 | 4M2-C9 heavy | QVTLKECGPGILQPSQTLSLTCSFS |
| | chain FR1 | |
| 92 | 4M2-C9, 5M1-A11 | VSWIRQPSGKGLEWLAH |
| | heavy chain FR2 | |
| 93 | 4M2-C9, 5M1-A11 | RYNPSLKSRLTISKDSSSNQVFLKITSVDTADTATYYC |
| | heavy chain FR3 | |
| 94 | 4M2-C9 heavy | WGQGTTLTVSS |
| | chain FR4 | |
| 95 | 4M2-C9 light | DIVMTQSPSSLSASLGDTVTITCHASQNVNVWLSWYQQKPGNIPKLLIYKASNLHAGVPSRFSGS |
| | chain | GSGTGFTLTISSLQPEDIATYYCQQGQSYPLTFGGGTKLEIK |
| | variable region | |
| 96 | 4M2-C9 light | QNVNVW |
| | chain CDR1 | |
| 97 | 4M2-C9 light | KAS |
| | chain CDR2 | |
| 98 | 4M2-C9 light | QQGQSYPLT |
| | chain CDR3 | |
| 99 | 4M2-C9 light | DIVMTQSPSSLSASLGDTVTITCHAS |
| | chain FR1 | |
| 100 | 4M2-C9 light | LSWYQQKPGNIPKLLIY |
| | chain FR2 | |
| 101 | 4M2-C9 light | NLHAGVPSRFSGSGSGTGFTLTISSLQPEDIATYYC |
| | chain FR3 | |
| 102 | 2M1-D2 heavy | QVQLQQSGAELVRPGTSVKISCKASGYTFTNYWLGWVKERAGHGLEWIGEIFPGGGHTNYKEKF |
| | chain variable | KGKATLTADTSSSTAYMKLSSLTSEDSAVYFCAQIPLYYGHYRSAYWGQGTLVTVSA |
| | region | |
| 103 | 2M1-D2, 13D5p, | QVQLQQSGAELVRPGTSVKISCKAS |
| | 13D5-1, 13D5-13 | |
| | heavy chain FR1 | |
| 104 | 2M1-D2 light chain | DIVMTQSPASLSASVGETVAITCGASENIYGALNWYQRKQGKSPQLLIYGATNLADGMSSRFSGS |
| | variable region | GSGRQYSLKISSLHPDDVATYYCQNVLSTPYTFGGGTKLEIK |
| 105 | 2M1-D2 light chain | DIVMTQSPASLSASVGETVAITCGAS |
| | FR1 | |
| 106 | 4M2-D9 heavy | EVQLQESGPGLVKPSQSLSLTCTVTGYSITSDYAWNWIRQFPGNRLEWMGFISYSGFTTYSPSLE |
| | chain variable | SRISITRDTSKNQFFLQLISVTTEDTATYYCARNHYGGSYWYFDVWGAGTSVTVSS |
| | region | |
| 107 | 4M2-D9 heavy | ISYSGFT |
| | chain CDR2 | |
| 108 | 4M2-D9 heavy | ARNHYGGSYWYFDV |
| | chain CDR3 | |
| 109 | 4M2-D9 heavy | EVQLQESGPGLVKPSQSLSLTCTVT |
| | chain FR1 | |
| 110 | 4M2-D9 heavy | WNWIRQFPGNRLEWMGF |
| | chain FR2 | |
| 111 | 4M2-D9 heavy | TYSPSLESRISITRDTSKNQFFLQLISVTTEDTATYYC |
| | chain FR3 | |
| 112 | 4M2-D9 heavy | WGAGTSVTVSS |
| | chain FR4 | |
| 113 | 4M2-D9 light | DIVMTQSPGSLAVSLGQRATISCRASESVEYYGTSLMQWYLQKPGQPPKLLIYAASNVESGVPDR |
| | chain | FRGSGSGTDFSLNIHPVEEDDIAMYFCQQSRKVPWTFGGGTKLEIK |
| | variable region | |
| 114 | 4M2-D9 light | ESVEYYGTSL |
| | chain CDR1 | |
| 115 | 4M2-D9 light | QQSRKVPWT |
| | chainCDR3 | |
| 116 | 4M2-D9 light | DIVMTQSPGSLAVSLGQRATISCRAS |
| | chainF R1 | |
| 117 | 4M2-D9 light | MQWYLQKPGQPPKLLIY |
| | chain FR2 | |
| 118 | 4M2-D9 light | NVESGVPDRFRGSGSGTDFSLNIHPVEEDDIAMYFC |
| | chain FR3 | |
| 119 | 1M2-D2 heavy | EVKVEESGGGLVQPGGSMKLSCAASGFTFSDAWMDWVHQSPEKGLEWVAEIRSKANNHATYYV |
| | chain variable | ESVEGRFTISRDDSKSSVFLQVNSLRPEDTGIYYCTRRDGYYFAYWGQGTLVTVSA |
| | region | |
| 120 | 1M2-D2 heavy | GFTFSDAW |
| | chain CDR1 | |
| 121 | 1M2-D2 heavy | IRSKANNHAT |
| | chain CDR2 | |
| 122 | 1M2-D2 heavy | TRRDGYYFAY |
| | chain CDR3 | |
| 123 | 1 M2-D2 heavy | EVKVEESGGGLVQPGGSMKLSCAAS |
| | chain FR1 | |
| 124 | 1M2-D2 heavy | MDWVHQSPEKGLEWVAE |
| | chain FR2 | |
| 125 | 1M2-D2 heavy | YYVESVEGRFTISRDDSKSSVFLQVNSLRPEDTGIYYC |
| | chain FR3 | |
| 126 | 1M2-D2 light | DIVLTQSPAIMSASLGEEITLTCSASSSVRDMHWYQQKSGTSPKVLIYNTFNLASGVPSRFSGSGS |
| | chain | GTFYSLTISSVEAGDAAAYYCHQWSSYPTFGGGTKLEIK |
| | variable region | |
| 127 | 1M2-D2 light | SSSVRD |
| | chain CDR1 | |
| 128 | 1M2-D2 light | NTF |
| | chain CDR2 | |
| 129 | 1M2-D2 light | HQWSSYPT |
| | chain CDR3 | |
| 130 | 1M2-D2 light | DIVLTQSPAIMSASLGEEITLTCSA |
| | chain FR1 | |
| 131 | 1M2-D2 light | MHWYQQKSGTSPKVLIY |
| | chain FR2 | |
| 132 | 1M2-D2 light | NLASGVPSRFSGSGSGTFYSLTISSVEAGDAAAYYC |
| | chain FR3 | |
| 133 | 5M1-A11 heavy | QVTLKVSGPGILQPSQTLSLTCSFSGFSLSTSGMGVSWIRQPSGKGLEWLAHIYWDDDKRYNPSL |
| | chain variable | KSRLTISKDSSSNQVFLKITSVDTADTATYYCARRLDGYNDPYYFDYWGQGTTLTVSS |
| | region | |
| 134 | 5M1-A11 heavy | QVTLKVSGPGILQPSQTLSLTCSFS |
| | chain FR1 | |
| 135 | 5M1-A11, 4M2-D5, | WGQGTTLTVSS |
| | 4M2-A8 heavy | |
| | chain FR4 | |
| 136 | 5M1-A11 light | DVVMTQTPALMSASPGEKVTMTCSASSSVSYMYWYQQKPRSSPKPWIYLTSNLASGVPARFSG |
| | chain | SGSGTSYSLTISSMEAEDAATYYCQQWNSNPYTFGGGTKLEIK |
| | variable region | |
| 137 | 5M1-A11 light | SSVSY |
| | chain CDR1 | |
| 138 | 5M1-A11 light | LTS |
| | chain CDR2 | |
| 139 | 5M1-A11 light | QQWNSNPYT |
| | chain CDR3 | |
| 140 | 5M1-A11 light | DVVMTQTPALMSASPGEKVTMTCSAS |
| | chain FR1 | |
| 141 | 5M1-A11 light | MYWYQQKPRSSPKPWIY |
| | chain FR2 | |
| 142 | 5M1-A11 light | NLASGVPARFSGSGSGTSYSLTISSMEAEDAATYYC |
| | chain FR3 | |
| 143 | 4M2-D5 heavy | QVQLQQSGAELARPGASVRMSCKASGYTFTSYTMNWVKQRPGQGLEWIGFINPDSDYTTYDQK |
| | chain variable | FKDKATLTADSSSSTAYMQLSSLTYDDSAVYYCTRHSYGNYGDYWGQGTTLTVSS |
| | region | |
| 144 | 4M2-D5 heavy | GYTFTSYT |
| | chain CDR1 | |
| 145 | 4M2-D5 heavy | INPDSDYT |
| | chain CDR2 | |
| 146 | 4M2-D5 heavy | TRHSYGNYGDY |
| | chain CDR3 | |
| 147 | 4M2-D5 heavy | QVQLQQSGAELARPGASVRMSCKAS |
| | chain FR1 | |
| 148 | 4M2-D5 heavy | MNWVKQRPGQGLEWIGF |
| | chain FR2 | |
| 149 | 4M2-D5 heavy | TYDQKFKDKATLTADSSSSTAYMQLSSLTYDDSAVYYC |
| | chain FR3 | |
| 150 | 4M2-D5 light | DIVLTQSPKFLLVSAGDRVTITCKASQSVTNDVAWYQQKPGQSPKLLIYYASSRYTGVPDRFTGS |
| | chain | GFGTDFTFTINTVQAEDLAVYFCQQDYSSPYTFGGGTKLEIK |
| | variable region | |
| 151 | 4M2-D5 light | QSVTND |
| | chain CDR1 | |
| 152 | 4M2-D5, 4M2-A8 | YAS |
| | light chain CDR2 | |
| 153 | 4M2-D5, 4M2-A8 | QQDYSSPYT |
| | light chain CDR3 | |
| 154 | 4M2-D5 light | DIVLTQSPKFLLVSAGDRVTITCKAS |
| | chain FR1 | |
| 155 | 4M2-D5, 4M2-A8 | VAWYQQKPGQSPKLLIY |
| | light chain FR2 | |
| 156 | 4M2-D5 light | SRYTGVPDRFTGSGFGTDFTFTINTVQAEDLAVYFC |
| | chain FR3 | |
| 157 | 4M2-A8 heavy | QVQLQQSGADLARPGASVKMSCKASGYTFIDYTVHWVKQRPGQGLEWIGFINPSNDYTSYNQKF |
| | chain variable | KDKASLTADTSSTTAYMQLSSLTSDDSAVYYCARHSYGNYGDYWGQGTTLTVSS |
| | region | |
| 158 | 4M2-A8 heavy | GYTFIDYT |
| | chain CDR1 | |
| 159 | 4M2-A8 heavy | INPSNDYT |
| | chain CDR2 | |
| 160 | 4M2-A8 heavy | ARHSYGNYGDY |
| | chain CDR3 | |
| 161 | 4M2-A8 heavy | QVQLQQSGADLARPGASVKMSCKAS |
| | chain FR1 | |
| 162 | 4M2-A8 heavy | VHWVKQRPGQGLEWIGF |
| | chain FR2 | |
| 163 | 4M2-A8 heavy | SYNQKFKDKASLTADTSSTTAYMQLSSLTSDDSAVYYC |
| | chain FR3 | |
| 164 | 4M2-A8 light | DIVMTQAPKFLLVSAGDRVTITCKASQSVTNGVAWYQQKPGQSPKLLIYYASNRYTGVPDRFTGS |
| | chain | GFGTDFTFTISTVQAEDLAVYFCQQDYSSPYTFGGGTKLEIK |
| | variable region | |
| 165 | 4M2-A8 light | QSVTNG |
| | chain CDR1 | |
| 166 | 4M2-A8 light | DIVMTQAPKFLLVSAGDRVTITCKAS |
| | chain FR1 | |
| 167 | 4M2-A8 light | NRYTGVPDRFTGSGFGTDFTFTISTVQAEDLAVYFC |
| | chain FR3 | |
| 168 | 9M2-C12 heavy | QVQLQQSGAELVKPGASVRLSCKASGYTFTSYWMHWVRQRPGLGLEWIGEIDPSDSYTNCNQR |
| | chain variable | FKGKATLTVDKSSSTAYTQLSSLTSEDSAVYYCARWAYGPYAMDYWGQGTSVTVSS |
| | region | |
| 169 | 9M2-C12 heavy | GYTFTSYW |
| | chain CDR1 | |
| 170 | 9M2-C12 heavy | IDPSDSYT |
| | chain CDR2 | |
| 171 | 9M2-C12 heavy | ARWAYGPYAMDY |
| | chain CDR3 | |
| 172 | 9M2-C12 heavy | QVQLQQSGAELVKPGASVRLSCKAS |
| | chain FR1 | |
| 173 | 9M2-C12 heavy | MHWVRQRPGLGLEWIGE |
| | chain FR2 | |
| 174 | 9M2-C12 heavy | NCNQRFKGKATLTVDKSSSTAYTQLSSLTSEDSAVYYC |
| | chain FR3 | |
| 175 | 9M2-C12 heavy | WGQGTSVTVSS |
| | chain FR4 | |
| 176 | 9M2-C12 light | DIVMTQTPLSLPVSLGDQASISCRSSQSLVHSNGNTYLHWYLQKPGQSPKLLIYKVSNRFSGVPD |
| | chain | RFSGSGSGTDFTLKISRVEAEDLGVYFCSQSTHVPWTFGGGTKLEIK |
| | variable region | |
| 177 | 9M2-C12 light | QSLVHSNGNTY |
| | chain CDR1 | |
| 178 | 9M2-C12, 13D5p, | KVS |
| | 13D5-1, 13D5-13 | |
| | light chain CDR2 | |
| 179 | 9M2-C12 light | SQSTHVPWT |
| | chain CDR3 | |
| 180 | 9M2-C12 light | DIVMTQTPLSLPVSLGDQASISCRSS |
| | chain FR1 | |
| 181 | 9M2-C12 light | LHWYLQKPGQSPKLLIY |
| | chain FR2 | |
| 182 | 9M2-C12 light | NRFSGVPDRFSGSGSGTDFTLKISRVEAEDLGVYFC |
| | chain FR3 | |
| 183 | 13D5p heavy chain | QVQLQQSGAELVRPGTSVKISCKASGYTFTNYWLGWVKQRPGHGLEWIGDIYPGGGYTNYNEKF |
| | variable region | KGKATLTADTSSSTAYMQLSSLTSEDSAVYFCARGGYYYGSSWYFDVWGAGTTVTVSS |
| 184 | 13D5p, 13D5-1, | IYPGGGYT |
| | 13D5-13 heavy | |
| | chain CDR2 | |
| 185 | 13D5p heavy chain | ARGGYYYGSSWYFDV |
| | CDR3 | |
| 186 | 13D5p, 13D5-1, | LGWVKQRPGHGLEWIGD |
| | 13D5-13 heavy | |
| | chain FR2 | |
| 187 | 13D5p, 13D5-1 | NYNEKFKGKATLTADTSSSTAYMQLSSLTSEDSAVYFC |
| | heavy chain FR3 | |
| 188 | 13D5p light chain | DVLMTQTPLSLPVSLGDQASISCRSSQSIVHSNGNTYLEWYLQKPGQSPKLLIYKVSNRFSGVPD |
| | variable region | RFSGSGSGTDFTLKISRVEAEDLGVYYCFQGSHVPPTFGGGTKLEIK |
| 189 | 13D5p light chain | QSIVHSNGNTY |
| | CDR1 | |
| 190 | 13D5p, 13D5-1, | FQGSHVPPT |
| | 13D5-13 light | |
| | chain CDR3 | |
| 191 | 13D5p, 13D5-1, | DVLMTQTPLSLPVSLGDQASISCRSS |
| | 13D5-13 light | |
| | chain FR1 | |
| 192 | 13D5p, 13D5-13 | LEWYLQKPGQSPKLLIY |
| | light chain FR2 | |
| 193 | 13D5p, 13D5-1 | NRFSGVPDRFSGSGSGTDFTLKISRVEAEDLGVYYC |
| | light chain FR3 | |
| 194 | 13D5-1 heavy | QVQLQQSGAELVRPGTSVKISCKASGYTFTNYWLGWVKQRPGHGLEWIGDIYPGGGYTNYNEKF |
| | chain | KGKATLTADTSSSTAYMQLSSLTSEDSAVYFCVRSGYYYGSSWYFDVWGAGTTVTVSS |
| | variable region | |
| 195 | 13D5-1 heavy | VRSGYYYGSSWYFDV |
| | chain CDR3 | |
| 196 | 13D5-1 light | DVLMTQTPLSLPVSLGDQASISCRSSQSIVHSSGNTYLEWYLQKPDQSPKLLIYKVSNRFSGVPDR |
| | chain | FSGSGSGTDFTLKISRVEAEDLGVYYCFQGSHVPPTFGGGTKLEIK |
| | variable region | |
| 197 | 13D5-1 light | QSIVHSSGNTY |
| | chain CDR1 | |
| 198 | 13D5-1 light | LEWYLQKPDQSPKLLIY |
| | chain FR2 | |
| 199 | 13D5-13 heavy | QVQLQQSGAELVRPGTSVKISCKASGYTFTNYWLGWVKQRPGHGLEWIGDIYPGGGYTNYNEKF |
| | chain variable | KGKATLTADISSSTAYMQLSSLTSEDSADYFCVRGGYYYGSSWYFDVWGAGTTVTVSS |
| | region | |
| 200 | 13D5-13 heavy | VRGGYYYGSSWYFDV |
| | chain CDR3 | |
| 201 | 13D5-13 heavy | NYNEKFKGKATLTADISSSTAYMQLSSLTSEDSADYFC |
| | chain FR3 | |
| 202 | 13D5-13 light | DVLMTQTPLSLPVSLGDQASISCRSSQSTVHSIGNTYLEWYLQKPGQSPKLLIYKVSNRFSGVPDR |
| | chain | FSGSESGTDFTLKISRVEAEDLGVYYCFQGSHVPPTFGGGTKLEIK |
| | variable region | |
| 203 | 13D5-13 light | QSTVHSIGNTY |
| | chain CDR1 | |
| 204 | 13D5-13 light | NRFSGVPDRFSGSESGTDFTLKISRVEAEDLGVYYC |
| | chain FR3 | |
| 205 | Human IgG1 | ASTKGPSVFPLAPSSKSTSGGTAALGCLVKDYFPEPVTVSWNSGALTSGVHTFPAVLQSSGLYSL |
| | constant region | SSVVTVPSSSLGTQTYICNVNHKPSNTKVDKKVEPKSCDKTHTCPPCPAPELLGGPSVFLFPPKP |
| | (IGHG1; | KDTLMISRTPEVTCVVVDVSHEDPEVKFNWYVDGVEVHNAKTKPREEQYNSTYRVVSVLTVLHQ |
| | UniProt:P01857-1, | DWLNGKEYKCKVSNKALPAPIEKTISKAKGQPREPQVYTLPPSRDELTKNQVSLTCLVKGFYPSDI |
| | v1) | AVEWESNGQPENNYKTTPPVLDSDGSFFLYSKLTVDKSRWQQGNVFSCSVMHEALHNHYTQKS |
| | | LSLSPGK |
| 206 | CH1 IgG1 | ASTKGPSVFPLAPSSKSTSGGTAALGCLVKDYFPEPVTVSWNSGALTSGVHTFPAVLQSSGLYSL |
| | (positions | SSVVTVPSSSLGTQTYICNVNHKPSNTKVDKKV |
| | 1-98 of P01857-1, | |
| | v1) | |
| 207 | Hinge IgG1 | EPKSCDKTHTCP |
| | (positions 99-110 | |
| | of P01857-1, v1) | |
| 208 | CH2 IgG1 | PCPAPELLGGPSVFLFPPKPKDTLMISRTPEVTCVVVDVSHEDPEVKFNWYVDGVEVHNAKTKPR |
| | (positions | EEQYNSTYRVVSVLTVLHQDWLNGKEYKCKVSNKALPAPIEKTISKAK |
| | 111-223 of | |
| | P01857-1, v1) | |
| 209 | CH3 IgG1 | GQPREPQVYTLPPSRDELTKNQVSLTCLVKGFYPSDIAVEWESNGQPENNYKTTPPVLDSDGSF |
| | (positions | FLYSKLTVDKSRWQQGNVFSCSVMHEALHNHYTQKSLSLSPGK |
| | 224-330 of | |
| | P01857-1, v1) | |
| 210 | CH3 (D356E, | GQPREPQVYTLPPSREEMTKNQVSLTCLVKGFYPSDIAVEWESNGQPENNYKTTPPVLDSDGSF |
| | L358M; positions | FLYSKLTVDKSRWQQGNVFSCSVMHEALHNHYTQKSLSLSPGK |
| | numbered | |
| | according to EU | |
| | numbering) | |
| 211 | Cκ CL (IGCK; | RTVAAPSVFIFPPSDEQLKSGTASVVCLLNNFYPREAKVQWKVDNALQSGNSQESVTEQDSKDS |
| | UniProt: P01834-1, | TYSLSSTLTLSKADYEKHKVYACEVTHQGLSSPVTKSFNRGEC |
| | v2) | |
| 212 | 4M2-C12 VH-CH1- | EVKLVESGPGLVKPSQSLSLTCTVTGYSITSDYAWNWIRQFPGNKLEWMGYITYSGNISYNPSLR |
| | CH2-CH3 | SRISITRDTSKNQFFLQLNSVTPEDTATYSCARSLYYPWYFDVWGAGTTVTVSSASTKGPSVFPLA |
| | | PSSKSTSGGTAALGCLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQKSLSLSPGK |
| 213 | 4M2-C12 VL-Cκ | DIVITQTPAILSTSPGEKVTMTCRASSSVGYIHWYQQKPGSSPKPWIYATSNLASGVPARFSGSGS |
| | | GTSNSLTITRVEAEDAATYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 214 | 4M2-B4 VH-CH1- | EVMLVESGPGLVKPSQSLSLTCTVTGYSITSDYAWNWIRQFPGNKLEWMGYITYSGNISYNPSLR |
| | CH2-CH3 | SRISITRDTSKNQFFLQLNSVTPEDTATYSCARSLYYPWYFDVWGAGTTVTVSSASTKGPSVFPLA |
| | | PSSKSTSGGTAALGCLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQKSLSLSPGK |
| 215 | 4M2-B4 VL-Cκ | DIVLTQTTAILSTSPGEKVTMTCRASSSVGYIHWYQQKPGSSPKPWIYATSNLASGVPARFSGSG |
| | | SGTSNSLTITRVEAEDAATYYCQQWSSYPPITFGGGTKLEVKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 216 | V4H1 VH-CH1- | EVQLLESGGGLVQPGGSLRLSCAASGYTITSDYAMSWVRQAPGKGLEWVSVITYSGNISYADSV |
| | CH2-CH3 | KGRFTISRDNSKNTLYLQMNSLRAEDTAVYYCARSLYYPWYFDVWGAGTTVTVSSASTKGPSVF |
| | | PLAPSSKSTSGGTAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSLSLSPGK |
| 217 | V4H1 VL-Cκ | EIVITQSPATLSLSPGERATLSCRASSSVGYLAWYQQKPGQAPRPLIYDTSNRATGIPARFSGSGS |
| | | GTDNTLTISSLEPEDFAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 218 | V4H2 VH-CH1- | QVQLQESGPGLVKPSDTLSLTCTVSGYSITSDYAWSWIRQPPGKGLEWIGYITYSGNISYNPSLKS |
| | CH2-CH3 | RVTISVDTSKNQFSLKLSSVTAADTAVYDCARSLYYPWYFDVWGAGTTVTVSSASTKGPSVFPLA |
| | | PSSKSTSGGTAALGCLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQKSLSLSPGK |
| 219 | V4H2 VL-Cκ | DIQMTQSPSSLSASVGDRVTITCRASSSVGYLNWYQQKPGKAPKPLIYAASSLQSGVPSRFSGSG |
| | | SGTDNTLTISSLQPEDFATYYCQQWSSYPPITFGGGTKLEVKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 220 | 2M1-B12 VH-CH1- | EVQLQQSGAELVRPGTSVKTSCKASGYTFTNYWLGWVKERAGHGLEWIGEIFPGGGHTNYKEKF |
| | CH2-CH3 | KGKATLTADTSSSTAYMKLSSLTSEDSAVYFCAQIPLYYGHYRSAYWGQGTLVTVSAASTKGPSV |
| | | FPLAPSSKSTSGGTAALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQKSLSLSPGK |
| 221 | 2M1-B12 VL-Cκ | DIQMMQSPASLSASVGETVAITCGASENIYGALNWYQRKQGKSPQLLIYGATNLADGMSSRFSGS |
| | | GSGRQYSLKISSLHPDDVATYYCQNVLSTPYTFGGGTKLEIKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 222 | 4M2-C9 VH-CH1- | QVTLKECGPGILQPSQTLSLTCSFSGFSLSTSGMGVSWIRQPSGKGLEWLAHIYWDDDKRYNPSL |
| | CH2-CH3 | KSRLTISKDSSSNQVFLKITSVDTADTATYYCARRLDGYNDPYYFDYWGQGTTLTVSSASTKGPS |
| | | VFPLAPSSKSTSGGTAALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGPSVFLFPPKPKDTLMISR |
| | | TPEVTCVVVDVSHEDPEVKFNWYVDGVEVHNAKTKPREEQYNSTYRVVSVLTVLHQDWLNGKE |
| | | YKCKVSNKALPAPIEKTISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEALHNHYTQKSLSLSPGK |
| 223 | 4M2-C9 VL-Cκ | DIVMTQSPSSLSASLGDTVTITCHASQNVNVWLSWYQQKPGNIPKLLIYKASNLHAGVPSRFSGS |
| | | GSGTGFTLTISSLQPEDIATYYCQQGQSYPLTFGGGTKLEIKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 224 | 2M1-D2 VH-CH1- | QVQLQQSGAELVRPGTSVKISCKASGYTFTNYWLGWVKERAGHGLEWIGEIFPGGGHTNYKEKF |
| | CH2-CH3 | KGKATLTADTSSSTAYMKLSSLTSEDSAVYFCAQIPLYYGHYRSAYWGQGTLVTVSAASTKGPSV |
| | | FPLAPSSKSTSGGTAALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQKSLSLSPGK |
| 225 | 2M1-D2 VL-Cκ | DIVMTQSPASLSASVGETVAITCGASENIYGALNWYQRKQGKSPQLLIYGATNLADGMSSRFSGS |
| | | GSGRQYSLKISSLHPDDVATYYCQNVLSTPYTFGGGTKLEIKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 226 | 4M2-D9 VH-CH1- | EVQLQESGPGLVKPSQSLSLTCTVTGYSITSDYAWNWIRQFPGNRLEWMGFISYSGFTTYSPSLE |
| | CH2-CH3 | SRISITRDTSKNQFFLQLISVTTEDTATYYCARNHYGGSYWYFDVWGAGTSVTVSSASTKGPSVF |
| | | PLAPSSKSTSGGTAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSLSLSPGK |
| 227 | 4M2-D9 VL-Cκ | DIVMTQSPGSLAVSLGQRATISCRASESVEYYGTSLMQWYLQKPGQPPKLLIYAASNVESGVPDR |
| | | FRGSGSGTDFSLNIHPVEEDDIAMYFCQQSRKVPWTFGGGTKLEIKRTVAAPSVFIFPPSDEQLKS |
| | | GTASVVCLLNNFYPREAKVQWKVDNALQSGNSQESVTEQDSKDSTYSLSSTLTLSKADYEKHKV |
| | | YACEVTHQGLSSPVTKSFNRGEC |
| 228 | 1M2-D2 VH-CH1- | EVKVEESGGGLVQPGGSMKLSCAASGFTFSDAWMDWVHQSPEKGLEWVAEIRSKANNHATYYV |
| | CH2-CH3 | ESVEGRFTISRDDSKSSVFLQVNSLRPEDTGIYYCTRRDGYYFAYWGQGTLVTVSAASTKGPSVF |
| | | PLAPSSKSTSGGTAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSLSLSPGK |
| 229 | 1M2-D2 VL-Cκ | DIVLTQSPAIMSASLGEEITLTCSASSSVRDMHWYQQKSGTSPKVLIYNTFNLASGVPSRFSGSGS |
| | | GTFYSLTISSVEAGDAAAYYCHQWSSYPTFGGGTKLEIKRTVAAPSVFIFPPSDEQLKSGTASVVC |
| | | LLNNFYPREAKVQWKVDNALQSGNSQESVTEQDSKDSTYSLSSTLTLSKADYEKHKVYACEVTH |
| | | QGLSSPVTKSFNRGEC |
| 230 | 5M1-A11 VH-CH1- | QVTLKVSGPGILQPSQTLSLTCSFSGFSLSTSGMGVSWIRQPSGKGLEWLAHIYWDDDKRYNPSL |
| | CH2-CH3 | KSRLTISKDSSSNQVFLKITSVDTADTATYYCARRLDGYNDPYYFDYWGQGTTLTVSSASTKGPS |
| | | VFPLAPSSKSTSGGTAALGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLGGPSVFLFPPKPKDTLMISR |
| | | TPEVTCVVVDVSHEDPEVKFNWYVDGVEVHNAKTKPREEQYNSTYRVVSVLTVLHQDWLNGKE |
| | | YKCKVSNKALPAPIEKTISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEALHNHYTQKSLSLSPGK |
| 231 | 5M1-A11 VL-Cκ | DVVMTQTPALMSASPGEKVTMTCSASSSVSYMYWYQQKPRSSPKPWIYLTSNLASGVPARFSG |
| | | SGSGTSYSLTISSMEAEDAATYYCQQWNSNPYTFGGGTKLEIKRTVAAPSVFIFPPSDEQLKSGT |
| | | ASVVCLLNNFYPREAKVQWKVDNALQSGNSQESVTEQDSKDSTYSLSSTLTLSKADYEKHKVYA |
| | | CEVTHQGLSSPVTKSFNRGEC |
| 232 | 4M2-D5 VH-CH1- | QVQLQQSGAELARPGASVRMSCKASGYTFTSYTMNWVKQRPGQGLEWIGFINPDSDYTTYDQK |
| | CH2-CH3 | FKDKATLTADSSSSTAYMQLSSLTYDDSAVYYCTRHSYGNYGDYWGQGTTLTVSSASTKGPSVF |
| | | PLAPSSKSTSGGTAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSLSLSPGK |
| 233 | 4M2-D5 VL-Cκ | DIVLTQSPKFLLVSAGDRVTITCKASQSVTNDVAWYQQKPGQSPKLLIYYASSRYTGVPDRFTGS |
| | | GFGTDFTFTINTVQAEDLAVYFCQQDYSSPYTFGGGTKLEIKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 234 | 4M2-A8 VH-CH1- | QVQLQQSGADLARPGASVKMSCKASGYTFIDYTVHWVKQRPGQGLEWIGFINPSNDYTSYNQKF |
| | CH2-CH3 | KDKASLTADTSSTTAYMQLSSLTSDDSAVYYCARHSYGNYGDYWGQGTTLTVSSASTKGPSVFP |
| | | LAPSSKSTSGGTAALGCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLHQDWLNGKEYKC |
| | | KVSNKALPAPIEKTISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNHYTQKSLSLSPGK |
| 235 | 4M2-A8 VL-Cκ | DIVMTQAPKFLLVSAGDRVTITCKASQSVTNGVAWYQQKPGQSPKLLIYYASNRYTGVPDRFTGS |
| | | GFGTDFTFTISTVQAEDLAVYFCQQDYSSPYTFGGGTKLEIKRTVAAPSVFIFPPSDEQLKSGTAS |
| | | VVCLLNNFYPREAKVQWKVDNALQSGNSQESVTEQDSKDSTYSLSSTLTLSKADYEKHKVYACE |
| | | VTHQGLSSPVTKSFNRGEC |
| 236 | 9M2-C12 VH-CH1- | QVQLQQSGAELVKPGASVRLSCKASGYTFTSYWMHWVRQRPGLGLEWIGEIDPSDSYTNCNQR |
| | CH2-CH3 | FKGKATLTVDKSSSTAYTQLSSLTSEDSAVYYCARWAYGPYAMDYWGQGTSVTVSSASTKGPSV |
| | | FPLAPSSKSTSGGTAALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQKSLSLSPGK |
| 237 | 9M2-C12 VL-Cκ | DIVMTQTPLSLPVSLGDQASISCRSSQSLVHSNGNTYLHWYLQKPGQSPKLLIYKVSNRFSGVPD |
| | | RFSGSGSGTDFTLKISRVEAEDLGVYFCSQSTHVPWTFGGGTKLEIKRTVAAPSVFIFPPSDEQLK |
| | | SGTASVVCLLNNFYPREAKVQWKVDNALQSGNSQESVTEQDSKDSTYSLSSTLTLSKADYEKHK |
| | | VYACEVTHQGLSSPVTKSFNRGEC |
| 238 | 13D5p VH-CH1- | QVQLQQSGAELVRPGTSVKISCKASGYTFTNYWLGWVKQRPGHGLEWIGDIYPGGGYTNYNEKF |
| | CH2-CH3 | KGKATLTADTSSSTAYMQLSSLTSEDSAVYFCARGGYYYGSSWYFDVWGAGTTVTVSSASTKGP |
| | | SVFPLAPSSKSTSGGTAALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGPSVFLFPPKPKDTLMIS |
| | | RTPEVTCVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEALHNHYTQKSLSLSPGK |
| 239 | 13D5p VL-Cκ | DVLMTQTPLSLPVSLGDQASISCRSSQSIVHSNGNTYLEWYLQKPGQSPKLLIYKVSNRFSGVPD |
| | | RFSGSGSGTDFTLKISRVEAEDLGVYYCFQGSHVPPTFGGGTKLEIKRTVAAPSVFIFPPSDEQLK |
| | | SGTASVVCLLNNFYPREAKVQWKVDNALQSGNSQESVTEQDSKDSTYSLSSTLTLSKADYEKHK |
| | | VYACEVTHQGLSSPVTKSFNRGEC |
| 240 | 13D5-1 VH-CH1- | QVQLQQSGAELVRPGTSVKISCKASGYTFTNYWLGWVKQRPGHGLEWIGDIYPGGGYTNYNEKF |
| | CH2-CH3 | KGKATLTADTSSSTAYMQLSSLTSEDSAVYFCVRSGYYYGSSWYFDVWGAGTTVTVSSASTKGP |
| | | SVFPLAPSSKSTSGGTAALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGPSVFLFPPKPKDTLMIS |
| | | RTPEVTCVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEALHNHYTQKSLSLSPGK |
| 241 | 13D5-1 VL-Cκ | DVLMTQTPLSLPVSLGDQASISCRSSQSIVHSSGNTYLEWYLQKPDQSPKLLIYKVSNRFSGVPDR |
| | | FSGSGSGTDFTLKISRVEAEDLGVYYCFQGSHVPPTFGGGTKLEIKRTVAAPSVFIFPPSDEQLKS |
| | | GTASVVCLLNNFYPREAKVQWKVDNALQSGNSQESVTEQDSKDSTYSLSSTLTLSKADYEKHKV |
| | | YACEVTHQGLSSPVTKSFNRGEC |
| 242 | 13D5-13 VH-CH1- | QVQLQQSGAELVRPGTSVKISCKASGYTFTNYWLGWVKQRPGHGLEWIGDIYPGGGYTNYNEKF |
| | CH2-CH3 | KGKATLTADISSSTAYMQLSSLTSEDSADYFCVRGGYYYGSSWYFDVWGAGTTVTVSSASTKGP |
| | | SVFPLAPSSKSTSGGTAALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLGGPSVFLFPPKPKDTLMIS |
| | | RTPEVTCVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEALHNHYTQKSLSLSPGK |
| 243 | 13D5-13 VL-CK | DVLMTQTPLSLPVSLGDQASISCRSSQSTVHSIGNTYLEWYLQKPGQSPKLLIYKVSNRFSGVPDR |
| | | FSGSESGTDFTLKISRVEAEDLGVYYCFQGSHVPPTFGGGTKLEIKRTVAAPSVFIFPPSDEQLKS |
| | | GTASVVCLLNNFYPREAKVQWKVDNALQSGNSQESVTEQDSKDSTYSLSSTLTLSKADYEKHKV |
| | | YACEVTHQGLSSPVTKSFNRGEC |
| 244 | 4M2- | GYX₁ITSDYA |
| | C12/V4H1/V4H2 | wherein X₁ = S or T |
| | heavy chain CDR1 | |
| | consensus | |
| 245 | 4M2- | X₂X₃S |
| | C12/V4H1/V4H2 | wherein X₂ = A or D; _{X3} = T or A |
| | light chain CDR2 | |
| | consensus | |
| 246 | 13D5p derived | X₄RX₅GYYYGSSWYFDV |
| | heavy chain CDR3 | wherein X₄ = V or A; X₅ = G or S |
| | consensus | |
| 247 | 13D5p derived | QSX₆VHSX₇GNTY |
| | light chain CDR1 | wherein X₆ = I or T; X₇ = N, S or I |
| | consensus | |
| 248 | 4M2-C12 mIgG2a | EVKLVESGPGLVKPSQSLSLTCTVTGYSITSDYAWNWIRQFPGNKLEWMGYITYSGNISYNPSLR |
| | HC | SRISITRDTSKNQFFLQLNSVTPEDTATYSCARSLYYPWYFDVWGAGTTVTVSSAKTTAPSVYPLA |
| | | PVCGDTTGSSVTLGCLVKGYFPEPVTLTWNSGSLSSGVHTFPAVLQSDLYTLSSSVTVTSSTWPS |
| | | QSITCNVAHPASSTKVDKKIEPRGPTIKPCPPCKCPAPNLLGGPSVFIFPPKIKDVLMISLSPIVTCV |
| | | VVDVSEDDPDVQISWFVNNVEVHTAQTQTHREDYNSTLRVVSALPIQHQDWMSGKEFKCKVNNK |
| | | DLPAPIERTISKPKGSVRAPQVYVLPPPEEEMTKKQVTLTCMVTDFMPEDIYVEWTNNGKTELNYK |
| | | NTEPVLDSDGSYFMYSKLRVEKKNWVERNSYSCSVVHEGLHNHHTTKSFSRTPGK |
| 249 | 4M2-C12 mIgG2a | EVKLVESGPGLVKPSQSLSLTCTVTGYSITSDYAWNWIRQFPGNKLEWMGYITYSGNISYNPSLR |
| | LALA PG HC | SRISITRDTSKNQFFLQLNSVTPEDTATYSCARSLYYPWYFDVWGAGTTVTVSSAKTTAPSVYPLA |
| | | PVCGDTTGSSVTLGCLVKGYFPEPVTLTWNSGSLSSGVHTFPAVLQSDLYTLSSSVTVTSSTWPS |
| | | QSITCNVAHPASSTKVDKKIEPRGPTIKPCPPCKCPAPNAAGGPSVFIFPPKIKDVLMISLSPIVTCV |
| | | VVDVSEDDPDVQISWFVNNVEVHTAQTQTHREDYNSTLRVVSALPIQHQDWMSGKEFKCKVNNK |
| | | DLGAPIERTISKPKGSVRAPQVYVLPPPEEEMTKKQVTLTCMVTDFMPEDIYVEWTNNGKTELNY |
| | | KNTEPVLDSDGSYFMYSKLRVEKKNWVERNSYSCSVVHEGLHNHHTTKSFSRTPGK |
| 250 | 4M2-C12 LC | DIVITQTPAILSTSPGEKVTMTCRASSSVGYIHWYQQKPGSSPKPWIYATSNLASGVPARFSGSGS |
| | | GTSNSLTITRVEAEDAATYYCQQWSSYPPITFGGGTKLEVKRADAAPTVSIFPPSSEQLTSGGASV |
| | | VCFLNNFYPKDINVKWKIDGSERQNGVLNSWTDQDSKDSTYSMSSTLTLTKDEYERHNSYTCEA |
| | | THKTSTSPIVKSFNRNEC |
| 251 | mIgG2a CH1 | AKTTAPSVYPLAPVCGDTTGSSVTLGCLVKGYFPEPVTLTWNSGSLSSGVHTFPAVLQSDLYTLS |
| | | SSVTVTSSTWPSQSITCNVAHPASSTKVDKKI |
| 252 | mIgG2a Hinge | EPRGPTIKPCPPCKCP |
| 253 | mIgG2a CH2 | APNLLGGPSVFIFPPKIKDVLMISLSPIVTCVVVDVSEDDPDVQISWFVNNVEVHTAQTQTHREDYN |
| | | STLRVVSALPIQHQDWMSGKEFKCKVNNKDLPAPIERTISKPK |
| 254 | mIgG2a CH2 LALA | APNAAGGPSVFIFPPKIKDVLMISLSPIVTCVVVDVSEDDPDVQISWFVNNVEVHTAQTQTHREDY |
| | PG | NSTLRVVSALPIQHQDWMSGKEFKCKVNNKDLGAPIERTISKPK |
| 255 | mIgG2a CH3 | GSVRAPQVYVLPPPEEEMTKKQVTLTCMVTDFMPEDIYVEWTNNGKTELNYKNTEPVLDSDGSY |
| | | FMYSKLRVEKKNWVERNSYSCSVVHEGLHNHHTTKSFSRTPGK |
| 256 | Mouse Ig gamma- | AKTTAPSVYPLAPVCGDTTGSSVTLGCLVKGYFPEPVTLTWNSGSLSSGVHTFPAVLQSDLYTLS |
| | 2A chain C region, | SSVTVTSSTWPSQSITCNVAHPASSTKVDKKIEPRGPTIKPCPPCKCPAPNLLGGPSVFIFPPKIKD |
| | A allele | VLMISLSPIVTCVVVDVSEDDPDVQISWFVNNVEVHTAQTQTHREDYNSTLRVVSALPIQHQDWM |
| | | SGKEFKCKVNNKDLPAPIERTISKPKGSVRAPQVYVLPPPEEEMTKKQVTLTCMVTDFMPEDIYVE |
| | | WTNNGKTELNYKNTEPVLDSDGSYFMYSKLRVEKKNWVERNSYSCSVVHEGLHNHHTTKSFSR |
| | | TPGK |
| 257 | mIgG2a CH2, CH3 | APNAAGGPSVFIFPPKIKDVLMISLSPIVTCVVVDVSEDDPDVQISWFVNNVEVHTAQTQTHREDY |
| | LALA PG | NSTLRVVSALPIQHQDWMSGKEFKCKVNNKDLGAPIERTISKPKGSVRAPQVYVLPPPEEEMTKK |
| | | QVTLTCMVTDFMPEDIYVEWTNNGKTELNYKNTEPVLDSDGSYFMYSKLRVEKKNWVERNSYSC |
| | | SVVHEGLHNHHTTKSFSRTPGK |
| 258 | 4M2-C12 mIgG2a | EVKLVESGPGLVKPSQSLSLTCTVTGYSITSDYAWNWIRQFPGNKLEWMGYITYSGNISYNPSLR |
| | NQ HC | SRISITRDTSKNQFFLQLNSVTPEDTATYSCARSLYYPWYFDVWGAGTTVTVSSAKTTAPSVYPLA |
| | | PVCGDTTGSSVTLGCLVKGYFPEPVTLTWNSGSLSSGVHTFPAVLQSDLYTLSSSVTVTSSTWPS |
| | | QSITCNVAHPASSTKVDKKIEPRGPTIKPCPPCKCPAPNLLGGPSVFIFPPKIKDVLMISLSPIVTCV |
| | | VVDVSEDDPDVQISWFVNNVEVHTAQTQTHREDYQSTLRVVSALPIQHQDWMSGKEFKCKVNN |
| | | KDLPAPIERTISKPKGSVRAPQVYVLPPPEEEMTKKQVTLTCMVTDFMPEDIYVEWTNNGKTELNY |
| | | KNTEPVLDSDGSYFMYSKLRVEKKNWVERNSYSCSVVHEGLHNHHTTKSFSRTPGK |
| 259 | mIgG2a CH2 NQ | APNLLGGPSVFIFPPKIKDVLMISLSPIVTCVVVDVSEDDPDVQISWFVNNVEVHTAQTQTHREDY |
| | | QSTLRVVSALPIQHQDWMSGKEFKCKVNNKDLPAPIERTISKPK |
| 260 | mIgG2a CH2, CH3 | APNLLGGPSVFIFPPKIKDVLMISLSPIVTCVVVDVSEDDPDVQISWFVNNVEVHTAQTQTHREDY |
| | NQ | QSTLRVVSALPIQHQDWMSGKEFKCKVNNKDLPAPIERTISKPKGSVRAPQVYVLPPPEEEMTKK |
| | | QVTLTCMVTDFMPEDIYVEWTNNGKTELNYKNTEPVLDSDGSYFMYSKLRVEKKNWVERNSYSC |
| | | SVVHEGLHNHHTTKSFSRTPGK |
| 261 | Mouse IGHG1 | AKTTPPSVYPLAPGSAAQTNSMVTLGCLVKGYFPEPVTVTWNSGSLSSGVHTFPAVLQSDLYTLS |
| | | SSVTVPSSTWPSETVTCNVAHPASSTKVDKKIVPRDCGCKPCICTVPEVSSVFIFPPKPKDVLTITL |
| | | TPKVTCVVVDISKDDPEVQFSWFVDDVEVHTAQTQPREEQFNSTFRSVSELPIMHQDWLNGKEF |
| | | KCRVNSAAFPAPIEKTISKTKGRPKAPQVYTIPPPKEQMAKDKVSLTCMITDFFPEDITVEWQWNG |
| | | QPAENYKNTQPIMNTNGSYFVYSKLNVQKSNWEAGNTFTCSVLHEGLHNHHTEKSLSHSPGK |
| 262 | mIgG1 CH1 | AKTTPPSVYPLAPGSAAQTNSMVTLGCLVKGYFPEPVTVTWNSGSLSSGVHTFPAVLQSDLYTLS |
| | | SSVTVPSSTWPSETVTCNVAHPASSTKVDKKI |
| 263 | mIgG1 Hinge | VPRDCGCKPCICT |
| 264 | mIgG1 CH2 | VPEVSSVFIFPPKPKDVLTITLTPKVTCVVVDISKDDPEVQFSWFVDDVEVHTAQTQPREEQFNST |
| | | FRSVSELPIMHQDWLNGKEFKCRVNSAAFPAPIEKTISKTK |
| 265 | mIgG1 CH3 | GRPKAPQVYTIPPPKEQMAKDKVSLTCMITDFFPEDITVEWQWNGQPAENYKNTQPIMNTNGSYF |
| | | VYSKLNVQKSNWEAGNTFTCSVLHEGLHNHHTEKSLSHSPGK |
| 266 | 4M2-C12 mIgG1 | EVKLVESGPGLVKPSQSLSLTCTVTGYSITSDYAWNWIRQFPGNKLEWMGYITYSGNISYNPSLR |
| | HC | SRISITRDTSKNQFFLQLNSVTPEDTATYSCARSLYYPWYFDVWGAGTTVTVSSAKTTPPSVYPLA |
| | | PGSAAQTNSMVTLGCLVKGYFPEPVTVTWNSGSLSSGVHTFPAVLQSDLYTLSSSVTVPSSTWP |
| | | SETVTCNVAHPASSTKVDKKIVPRDCGCKPCICTVPEVSSVFIFPPKPKDVLTITLTPKVTCVVVDIS |
| | | KDDPEVQFSWFVDDVEVHTAQTQPREEQFNSTFRSVSELPIMHQDWLNGKEFKCRVNSAAFPA |
| | | PIEKTISKTKGRPKAPQVYTIPPPKEQMAKDKVSLTCMITDFFPEDITVEWQWNGQPAENYKNTQP |
| | | IMNTNGSYFVYSKLNVQKSNWEAGNTFTCSVLHEGLHNHHTEKSLSHSPGK |
| 267 | IGN175A HC | QVQLQQSGAELMKPGASVKISCKATGYTFSTHWIEWVKQRPGHGLEWIGEILPGSGSTSYNEKF |
| | | KGKATFTADTSSNTAYMQLSSLTSEDSAVYYCARWLLYYYAMDYWGQGTSVTVSSASTKGPSVF |
| | | PLAPSSKSTSGGTAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEALHNHYTQKSLSLSPGK |
| 268 | IGN175A LC | DVLMTQTPLSLPVSLGDQASISCRSSQSIVHSNGNTYLEWYLQKPGQSPKLLIYKLSNRFSGVPDR |
| | | FSGSGSGTDFTLKISRVEAEDLGVYYCFQGSHFPYTFGGGTKLEIKRTVAAPSVFIFPPSDEQLKS |
| | | GTASVVCLLNNFYPREAKVQWKVDNALQSGNSQESVTEQDSKDSTYSLSSTLTLSKADYEKHKV |
| | | YACEVTHQGLSSPVTKSFNRGEC |
| 269 | VSTB112 HC | QVQLVQSGAEVKKPGSSVKVSCKASGGTFSSYAISWVRQAPGQGLEWMGGIIPIFGTANYAQKF |
| | | QGRVTITADESTSTAYMELSSLRSEDTAVYYCARSSYGWSYEFDYWGQGTLVTVSSASTKGPSV |
| | | FPLAPSSKSTSGGTAALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQKSLSLSPGK |
| 270 | VSTB112 LC | DIQMTQSPSSLSASVGDRVTITCRASQSIDTRLNWYQQKPGKAPKLLIYSASSLQSGVPSRFSGS |
| | | GSGTDFTLTISSLQPEDFATYYCQQSAYNPITFGQGTKVEIKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 271 | VSTB112 major | PVDKGHDVTF |
| | epitope 1 | |
| 272 | VSTB112 major | RRPIRNLTFQDL |
| | epitope 2 | |
| 273 | VSTB112 minor | TWYRSSRGEVQTCS |
| | epitope 1 | |
| 274 | VSTB112 minor | EIRHHHSEHRVHGAMEL |
| | epitope 2 | |
| 275 | IGN175A epitope | FKVATPYSLYVCPEGQNVTLTCRLLGPVDKGH |
| 276 | V4-C1 heavy chain | QVQLQESGPGLVKPSDTLSLTCTVSGYSITSDYAWNWIRQTPGKGLEWIGYITYSGYISYNPSLRS |
| | variable region | RVTISRDTSKNQFSLKLSSVTAADTAVYSCARALYYPWYFDVWGTGTTVTVSS |
| 277 | V4-C1 heavy chain | ITYSGYI |
| | CDR2 | |
| 278 | V4-C1, V4-C9, V4- | ARALYYPWYFDV |
| | C24, V4-C26, V4- | |
| | C27, V4-C28, V4- | |
| | C30, V4-C31 heavy | |
| | chain CDR3 | |
| 279 | V4-C1, V4-C9 | WNWIRQTPGKGLEWIGY |
| | heavy chain FR2 | |
| 280 | V4-C1, V4-C9 | SYNPSLRSRVTISRDTSKNQFSLKLSSVTAADTAVYSC |
| | heavy chain FR3 | |
| 281 | V4-C1, V4-C9, V4- | WGTGTTVTVSS |
| | C24, V4-C26, V4- | |
| | C27, V4-C28, V4- | |
| | C30, V4-C31 heavy | |
| | chain FR4 | |
| 282 | V4-C1 light chain | EIVITQSPATLSLSPGERATLSCRASSSVGYIHWYQQKPGQAPRPLIYATSNRATGIPARFSGSGS |
| | variable region | GTDNTLTISSLEPEDSAVYYCQQWSSYPPITFGGGTKLEVK |
| 283 | V4-C1, V4-C9, V4- | IHWYQQKPGQAPRPLIY |
| | C24, V4-C27, V4- | |
| | C28, V4-C30, V4- | |
| | C31 light chain | |
| | FR2 | |
| 284 | V4-C1, V4-C9, V4- | NRATGIPARFSGSGSGTDNTLTISSLEPEDSAVYYC |
| | C26, V4-C27 light | |
| | chain FR3 | |
| 285 | V4-C9 heavy chain | QVQLQESGPGLVKPSDTLSLTCTVSGYSITSDYAWNWIRQTPGKGLEWIGYITYSGYVSYNPSLR |
| | variable region | SRVTISRDTSKNQFSLKLSSVTAADTAVYSCARALYYPWYFDVWGTGTTVTVSS |
| 286 | V4-C9 heavy chain | ITYSGYV |
| | CDR2 | |
| 287 | V4-C9 light chain | EIVLTQSPATLSLSPGERATLSCRASSSVGYIHWYQQKPGQAPRPLIYATSNRATGIPARFSGSGS |
| | variable region | GTDNTLTISSLEPEDSAVYYCQQWSSYPPITFGGGTKLEVK |
| 288 | V4-C9, V4-C24, V4- | EIVLTQSPATLSLSPGERATLSCRAS |
| | C26, V4-C27, V4- | |
| | C28, V4-C30, V4- | |
| | C31 light chain | |
| | FR1 | |
| 289 | V4-C24, V4-C26, | QVQLQESGPGLVKPSDTLSLTCTVSGYSITSDYTWNWIRQTPGKGLEWIGHITYSGSVSYNPSLR |
| | V4-C27, V4-C28, | SRVTISRDTSKNQFSLKLSSVTAADTATYSCARALYYPWYFDVWGTGTTVTVSS |
| | V4-C30, V4-C31 | |
| | heavy chain | |
| | variable region | |
| 290 | V4-C24, V4-C26, | GYSITSDYT |
| | V4-C27, V4-C28, | |
| | V4-C30, V4-C31 | |
| | heavy chain CDR1 | |
| 291 | V4-C24, V4-C26, | ITYSGSV |
| | V4-C27, V4-C28, | |
| | V4-C30, V4-C31 | |
| | heavy chain CDR2 | |
| 292 | V4-C24, V4-C26, | WNWIRQTPGKGLEWIGH |
| | V4-C27, V4-C28, | |
| | V4-C30, V4-C31 | |
| | heavy chain FR2 | |
| 293 | V4-C24, V4-C26, | SYNPSLRSRVTISRDTSKNQFSLKLSSVTAADTATYSC |
| | V4-C27, V4-C28, | |
| | V4-C30, V4-C31 | |
| | heavy chain FR3 | |
| 294 | V4-C24 light | EIVLTQSPATLSLSPGERATLSCRASSSVGYIHWYQQKPGQAPRPLIYTTSYRATGIPARFSGSGS |
| | chain | GTDNTLTISSLEPEDSAVYYCQQWSSYPPITFGGGTKLEVK |
| | variable region | |
| 295 | V4-C24, V4-C26 | TTS |
| | light chain CDR2 | |
| 296 | V4-C24, V4-C28, | YRATGIPARFSGSGSGTDNTLTISSLEPEDSAVYYC |
| | V4-C30 light | |
| | chain FR3 | |
| 297 | V4-C26 light | EIVLTQSPATLSLSPGERATLSCRASSSVGYIHWYQQKPGQAPRPIIYTTSNRATGIPARFSGSGS |
| | chain | GTDNTLTISSLEPEDSAVYYCQQWSSYPPITFGGGTKLEVK |
| | variable region | |
| 298 | V4-C26 light | IHWYQQKPGQAPRPIIY |
| | chain FR2 | |
| 299 | V4-C27 light | EIVLTQSPATLSLSPGERATLSCRASSSVGYIHWYQQKPGQAPRPLIYATYNRATGIPARFSGSGS |
| | chain | GTDNTLTISSLEPEDSAVYYCQQWSSYPPITFGGGTKLEVK |
| | variable region | |
| 300 | V4-C27, V4-C30, | ATY |
| | V4-C31 light | |
| | chain CDR2 | |
| 301 | V4-C28 light | EIVLTQSPATLSLSPGERATLSCRASSSVGYIHWYQQKPGQAPRPLIYATSYRATGIPARFSGSGS |
| | chain | GTDNTLTISSLEPEDSAVYYCQQWSSYPPITFGGGTKLEVK |
| | variable region | |
| 302 | V4-C30 light | EIVLTQSPATLSLSPGERATLSCRASSSVGYIHWYQQKPGQAPRPLIYATYYRATGIPARFSGSGS |
| | chain | GTDNTLTISSLEPEDSAVYYCQQWSSYPPITFGGGTKLEVK |
| | variable region | |
| 303 | V4-C31 light | EIVLTQSPATLSLSPGERATLSCRASSSVGYIHWYQQKPGQAPRPLIYATYYRTTGIPARFSGSGS |
| | chain | GTDNTLTISSLEPEDSAVYYCQQWSSYPPITFGGGTKLEVK |
| | variable region | |
| 304 | V4-C31 light | YRTTGIPARFSGSGSGTDNTLTISSLEPEDSAVYYC |
| | chain FR3 | |
| 305 | 4M2-C12 derived | GYX₈ITSDYX₉ |
| | heavy chain CDR1 | wherein X₈ = S or T; X₉ = T or A |
| | consensus | |
| 306 | 4M2-C12 derived | ITYSGX₁₀X₁₁ |
| | heavy chain CDR2 | wherein X₁₀ = S, N or Y; X₁₁ = V or I |
| | consensus | |
| 307 | 4M2-C12 derived | ARX₁₂LYYPWYFDV |
| | heavy chain CDR3 | wherein X₁₂ = A or S |
| | consensus | |
| 308 | 4M2-C12 derived | X₁₃X₁₄X₁₅ |
| | light chain CDR2 | wherein X₁₃ = A, T or D; X₁₄ = T or A; X₁₅ = S or Y |
| | consensus | |
| 309 | C24/C26/C27 light | X₁₆TX₁₇ |
| | chain CDR2 | wherein X₁₆ = T or A; X₁₇ = S or Y |
| | consensus | |
| 310 | C24/C26/C27 light | EIVLTQSPATLSLSPGERATLSCRASSSVGYIHWYQQKPGQAPRPX₁₇LYX₁₈TX₁₉X₂₀YNRATGIPAR |
| | chain variable | FSGSGSGTDNTLTISSLEPEDSAVYYCQQWSSYPPITFGGGTKLEVK |
| | region consensus | wherein X₁₇ = L or I; X₁₈ = T or A; X₁₉ = S or Y; X₂₀ = N or Y |
| 311 | V4-C1 VH-CH1- | QVQLQESGPGLVKPSDTLSLTCTVSGYSITSDYAWNWIRQTPGKGLEWIGYITYSGYISYNPSLRS |
| | CH2-CH3 | RVTISRDTSKNQFSLKLSSVTAADTAVYSCARALYYPWYFDVWGTGTTVTVSSASTKGPSVFPLA |
| | | PSSKSTSGGTAALGCLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKV |
| | | SNKALPAPIEKTISKAKGQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQKSLSLSPGK |
| 312 | V4-C1 VL-Cκ | EIVITQSPATLSLSPGERATLSCRASSSVGYIHWYQQKPGQAPRPLIYATSNRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 313 | V4-C9 VH-CH1- | QVQLQESGPGLVKPSDTLSLTCTVSGYSITSDYAWNWIRQTPGKGLEWIGYITYSGYVSYNPSLR |
| | CH2-CH3 | SRVTISRDTSKNQFSLKLSSVTAADTAVYSCARALYYPWYFDVWGTGTTVTVSSASTKGPSVFPL |
| | | APSSKSTSGGTAALGCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYTQKSLSLSPGK |
| 314 | V4-C9 VL-Cκ | EIVLTQSPATLSLSPGERATLSCRASSSVGYIHWYQQKPGQAPRPLIYATSNRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 315 | V4- | QVQLQESGPGLVKPSDTLSLTCTVSGYSITSDYTWNWIRQTPGKGLEWIGHITYSGSVSYNPSLR |
| | C24/C26/C27/C28/ | SRVTISRDTSKNQFSLKLSSVTAADTATYSCARALYYPWYFDVWGTGTTVTVSSASTKGPSVFPL |
| | C30/C31 VH-CH1- | APSSKSTSGGTAALGCLVKDYFPEPVTVSWNSGALTSGVHTFPAVLQSSGLYSLSSVVTVPSSSL |
| | CH2-CH3 | GTQTYICNVNHKPSNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYTQKSLSLSPGK |
| 316 | V4-C24 VL-Cκ | EIVLTQSPATLSLSPGERATLSCRASSSVGYIHWYQQKPGQAPRPLIYTTSYRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 317 | V4-C26 VL-Cκ | EIVLTQSPATLSLSPGERATLSCRASSSVGYIHWYQQKPGQAPRPIIYTTSNRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 318 | V4-C27 VL-Cκ | EIVLTQSPATLSLSPGERATLSCRASSSVGYIHWYQQKPGQAPRPLIYATYNRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 319 | V4-C28 VL-Cκ | EIVLTQSPATLSLSPGERATLSCRASSSVGYIHWYQQKPGQAPRPLIYATSYRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 320 | V4-C30 VL-Cκ | EIVLTQSPATLSLSPGERATLSCRASSSVGYIHWYQQKPGQAPRPLIYATYYRATGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 321 | V4-C31 VL-Cκ | EIVLTQSPATLSLSPGERATLSCRASSSVGYIHWYQQKPGQAPRPLIYATYYRTTGIPARFSGSGS |
| | | GTDNTLTISSLEPEDSAVYYCQQWSSYPPITFGGGTKLEVKRTVAAPSVFIFPPSDEQLKSGTASV |
| | | VCLLNNFYPREAKVQWKVDNALQSGNSQESVTEQDSKDSTYSLSSTLTLSKADYEKHKVYACEV |
| | | THQGLSSPVTKSFNRGEC |
| 322 | VISTA sequence to | SRGEVQ |
| | which 4M2-C12 and | |
| | derivatives bind | |
| 323 | Human PSGL-1 | MPLQLLLLLILLGPGNSLQLWDTWADEAEKALGPLLARDRRQATEYEYLDYDFLPETEPPEMLRN |
| | isoform 1 | STDTTPLTGPGTPESTTVEPAARRSTGLDAGGAVTELTTELANMGNLSTDSAAMEIQTTQPAATE |
| | (UniProt: | AQTTQPVPTEAQTTPLAATEAQTTRLTATEAQTTPLAATEAQTTPPAATEAQTTQPTGLEAQTTAP |
| | Q14242-1, v1) | AAMEAQTTAPAAMEAQTTPPAAMEAQTTQTTAMEAQTTAPEATEAQTTQPTATEAQTTPLAAME |
| | | ALSTEPSATEALSMEPTTKRGLFIPFSVSSVTHKGIPMAASNLSVNYPVGAPDHISVKQCLLAILILA |
| | | LVATIFFVCTVVLAVRLSRKGHMYPVRNYSPTEMVCISSLLPDGGEGPSATANGGLSKAKSPGLTP |
| | | EPREDREGDDLTLHSFLP |
| 324 | Human PSGL-1 | MAVGASGLEGDKMAGAMPLQLLLLLILLGPGNSLQLWDTWADEAEKALGPLLARDRRQATEYEY |
| | isoform 2 | LDYDFLPETEPPEMLRNSTDTTPLTGPGTPESTTVEPAARRSTGLDAGGAVTELTTELANMGNLS |
| | (UniProt: | TDSAAMEIQTTQPAATEAQTTQPVPTEAQTTPLAATEAQTTRLTATEAQTTPLAATEAQTTPPAAT |
| | Q14242-2) | EAQTTQPTGLEAQTTAPAAMEAQTTAPAAMEAQTTPPAAMEAQTTQTTAMEAQTTAPEATEAQT |
| | | TQPTATEAQTTPLAAMEALSTEPSATEALSMEPTTKRGLFIPFSVSSVTHKGIPMAASNLSVNYPV |
| | | GAPDHISVKQCLLAILILALVATIFFVCTVVLAVRLSRKGHMYPVRNYSPTEMVCISSLLPDGGEGP |
| | | SATANGGLSKAKSPGLTPEPREDREGDDLTLHSFLP |
| 325 | Mature human | LQLWDTWADEAEKALGPLLARDRRQATEYEYLDYDFLPETEPPEMLRNSTDTTPLTGPGTPESTT |
| | PSGL-1 isoform 1 | VEPAARRSTGLDAGGAVTELTTELANMGNLSTDSAAMEIQTTQPAATEAQTTQPVPTEAQTTPLA |
| | (Q14242-1, v1 | ATEAQTTRLTATEAQTTPLAATEAQTTPPAATEAQTTQPTGLEAQTTAPAAMEAQTTAPAAMEAQ |
| | positions 18 to | TTPPAAMEAQTTQTTAMEAQTTAPEATEAQTTQPTATEAQTTPLAAMEALSTEPSATEALSMEPT |
| | 412) | TKRGLFIPFSVSSVTHKGIPMAASNLSVNYPVGAPDHISVKQCLLAILILALVATIFFVCTVVLAVRL |
| | | SRKGHMYPVRNYSPTEMVCISSLLPDGGEGPSATANGGLSKAKSPGLTPEPREDREGDDLTLHSF |
| | | LP |
| 326 | PSGL-1 | LQLWDTWADEAEKALGPLLARDRRQATEYEYLDYDFLPETEPPEMLRNSTDTTPLTGPGTPESTT |
| | extracellular | VEPAARRSTGLDAGGAVTELTTELANMGNLSTDSAAMEIQTTQPAATEAQTTQPVPTEAQTTPLA |
| | domain | ATEAQTTRLTATEAQTTPLAATEAQTTPPAATEAQTTQPTGLEAQTTAPAAMEAQTTAPAAMEAQ |
| | (Q14242-1, v1 | TTPPAAMEAQTTQTTAMEAQTTAPEATEAQTTQPTATEAQTTPLAAMEALSTEPSATEALSMEPT |
| | positions 18 to | TKRGLFIPFSVSSVTHKGIPMAASNLSVNYPVGAPDHISVKQC |
| | 320) | |
| 327 | PSGL-1 | LLAILILALVATIFFVCTVVL |
| | transmembrane | |
| | domain (Q14242-1, | |
| | v1 positions 321 | |
| | to3 41) | |
| 328 | PSGL-1 | AVRLSRKGHMYPVRNYSPTEMVCISSLLPDGGEGPSATANGGLSKAKSPGLTPEPREDREGDDL |
| | cytoplasmic | TLHSFLP |
| | domain | |
| | (Q14242-1, v1 | |
| | positions 342 to | |
| | 412) | |
| 329 | PSGL-1 | QTTQPAATEAQTTQPVPTEAQTTPLAATEAQTTRLTATEAQTTPLAATEAQTTPPAATEAQTTQPT |
| | extracellular | GLEAQTTAPAAMEAQTTAPAAMEAQTTPPAAMEAQTTQTTAMEAQTTAPEATEAQTTQPTATEA |
| | domain | QTTPLAAMEA |
| | repeat region | |
| | (Q14242-1, v1 | |
| | positions 122 to | |
| | 261) | |
| 330 | 4M2-C12 VH-CH1- | EVKLVESGPGLVKPSQSLSLTCTVTGYSITSDYAWNWIRQFPGNKLEWMGYITYSGNISYNPSLR |
| | CH2-CH3 IgG4 | SRISITRDTSKNQFFLQLNSVTPEDTATYSCARSLYYPWYFDVWGAGTTVTVSSASTKGPSVFPLA |
| | | PCSRSTSESTAALGCLVKDYFPEPVTVSWNSGALTSGVHTFPAVLQSSGLYSLSSVVTVPSSSLG |
| | | TKTYTCNVDHKPSNTKVDKRVESKYGPPCPSCPAPEFLGGPSVFLFPPKPKDTLMISRTPEVTCV |
| | | VVDVSQEDPEVQFNWYVDGVEVHNAKTKPREEQFNSTYRVVSVLTVLHQDWLNGKEYKCKVSN |
| | | KGLPSSIEKTISKAKGQPREPQVYTLPPSQEEMTKNQVSLTCLVKGFYPSDIAVEWESNGQPENN |
| | | YKTTPPVLDSDGSFFLYSRLTVDKSRWQEGNVFSCSVMHEALHNHYTQKSLSLSLGK |
| 331 | V4-C26 | QVQLQESGPGLVKPSDTLSLTCTVSGYSITSDYTWNWIRQTPGKGLEWIGHITYSGSVSYNPSLR |
| | hIgG4(S228P) HC | SRVTISRDTSKNQFSLKLSSVTAADTATYSCARALYYPWYFDVWGTGTTVTVSSASTKGPSVFPL |
| | | APCSRSTSESTAALGCLVKDYFPEPVTVSWNSGALTSGVHTFPAVLQSSGLYSLSSVVTVPSSSL |
| | | GTKTYTCNVDHKPSNTKVDKRVESKYGPPCPPCPAPEFLGGPSVFLFPPKPKDTLMISRTPEVTC |
| | | VVVDVSQEDPEVQFNWYVDGVEVHNAKTKPREEQFNSTYRVVSVLTVLHQDWLNGKEYKCKVS |
| | | NKGLPSSIEKTISKAKGQPREPQVYTLPPSQEEMTKNQVSLTCLVKGFYPSDIAVEWESNGQPEN |
| | | NYKTTPPVLDSDGSFFLYSRLTVDKSRWQEGNVFSCSVMHEALHNHYTQKSLSLSLGK |

### Numbered Paragraphs

The following numbered paragraphs (paras) provide further statements of features and combinations of features which are contemplated in connection with the present invention:

1. An antigen-binding molecule, optionally isolated, which is capable of binding to VISTA and inhibiting VISTA-mediated signalling, independently of Fc-mediated function.

2. The antigen-binding molecule according to para 1, which is capable of binding to VISTA in the Ig-like V-type domain.

3. The antigen-binding molecule according to para 1 or para 2, wherein the antigen-binding molecule is capable of binding to a polypeptide comprising or consisting of the amino acid sequence of SEQ ID NO:6.

4. The antigen-binding molecule according to any one of paras 1 to 3, wherein the antigen-binding molecule is capable of binding to a polypeptide comprising or consisting of the amino acid sequence of SEQ ID NO:31.

5. The antigen-binding molecule according to any one of paras 1 to 4, wherein the antigen-binding molecule does not compete with IGN175A for binding to VISTA.

6. The antigen-binding molecule according to any one of paras 1 to 5, wherein the antigen-binding molecule is not capable of binding to a peptide consisting of the amino acid sequence of SEQ ID NO:275.

7. The antigen-binding molecule according to any one of paras 1 to 6, wherein the antigen-binding molecule comprises:
(i) a heavy chain variable (VH) region incorporating the following CDRs:
   HC-CDR1 having the amino acid sequence of SEQ ID NO:305
   HC-CDR2 having the amino acid sequence of SEQ ID NO:306
   HC-CDR3 having the amino acid sequence of SEQ ID NO:307; and
(ii) a light chain variable (VL) region incorporating the following CDRs:
   LC-CDR1 having the amino acid sequence of SEQ ID NO:41
   LC-CDR2 having the amino acid sequence of SEQ ID NO:308
   LC-CDR3 having the amino acid sequence of SEQ ID NO:43.

8. The antigen-binding molecule according to any one of paras 1 to 7, wherein the antigen-binding molecule comprises:
(i) a heavy chain variable (VH) region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278; and
(ii) a light chain variable (VL) region incorporating the following CDRs:
   LC-CDR1 having the amino acid sequence of SEQ ID NO:41
   LC-CDR2 having the amino acid sequence of SEQ ID NO:309
   LC-CDR3 having the amino acid sequence of SEQ ID NO:43.

9. The antigen-binding molecule according to any one of paras 1 to 8, wherein the antigen-binding molecule comprises:
(i) a heavy chain variable (VH) region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278; and
(ii) a light chain variable (VL) region incorporating the following CDRs:
   LC-CDR1 having the amino acid sequence of SEQ ID NO:41
   LC-CDR2 having the amino acid sequence of SEQ ID NO:295
   LC-CDR3 having the amino acid sequence of SEQ ID NO:43.

10. The antigen-binding molecule according to any one of paras 1 to 8, wherein the antigen-binding molecule comprises:
(i) a heavy chain variable (VH) region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278; and
(ii) a light chain variable (VL) region incorporating the following CDRs:
   LC-CDR1 having the amino acid sequence of SEQ ID NO:41
   LC-CDR2 having the amino acid sequence of SEQ ID NO:300
   LC-CDR3 having the amino acid sequence of SEQ ID NO:43.

11. The antigen-binding molecule according to any one of paras 1 to 8, wherein the antigen-binding molecule comprises:
(i) a heavy chain variable (VH) region incorporating the following CDRs:
   HC-CDR1 having the amino acid sequence of SEQ ID NO:33
   HC-CDR2 having the amino acid sequence of SEQ ID NO:277
   HC-CDR3 having the amino acid sequence of SEQ ID NO:278; and
(ii) a light chain variable (VL) region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43.

12. The antigen-binding molecule according to any one of paras 1 to 8, wherein the antigen-binding molecule comprises:
(i) a heavy chain variable (VH) region incorporating the following CDRs:
   HC-CDR1 having the amino acid sequence of SEQ ID NO:33
   HC-CDR2 having the amino acid sequence of SEQ ID NO:286
   HC-CDR3 having the amino acid sequence of SEQ ID NO:278; and
(ii) a light chain variable (VL) region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43.

13. The antigen-binding molecule according to any one of paras 1 to 8, wherein the antigen-binding molecule comprises:
(i) a heavy chain variable (VH) region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278; and
(ii) a light chain variable (VL) region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43.

14. The antigen-binding molecule according to any one of paras 1 to 8, wherein the antigen-binding molecule comprises:
(i) a heavy chain variable (VH) region incorporating the following CDRs:
   HC-CDR1 having the amino acid sequence of SEQ ID NO:290
   HC-CDR2 having the amino acid sequence of SEQ ID NO:291
   HC-CDR3 having the amino acid sequence of SEQ ID NO:278; and
(ii) a light chain variable (VL) region incorporating the following CDRs:
   LC-CDR1 having the amino acid sequence of SEQ ID NO:41
   LC-CDR2 having the amino acid sequence of SEQ ID NO:300
   LC-CDR3 having the amino acid sequence of SEQ ID NO:43.

15. The antigen-binding molecule according to any one of paras 1 to 7, wherein the antigen-binding molecule comprises:
(i) a heavy chain variable (VH) region incorporating the following CDRs:
   HC-CDR1 having the amino acid sequence of SEQ ID NO:33
   HC-CDR2 having the amino acid sequence of SEQ ID NO:34
   HC-CDR3 having the amino acid sequence of SEQ ID NO:35; and
(ii) a light chain variable (VL) region incorporating the following CDRs:
   LC-CDR1 having the amino acid sequence of SEQ ID NO:41
   LC-CDR2 having the amino acid sequence of SEQ ID NO:42
   LC-CDR3 having the amino acid sequence of SEQ ID NO:43.

16. The antigen-binding molecule according to any one of paras 1 to 7, wherein the antigen-binding molecule comprises:
(i) a heavy chain variable (VH) region incorporating the following CDRs:
   HC-CDR1 having the amino acid sequence of SEQ ID NO:33
   HC-CDR2 having the amino acid sequence of SEQ ID NO:34
   HC-CDR3 having the amino acid sequence of SEQ ID NO:35; and
(ii) a light chain variable (VL) region incorporating the following CDRs:
   LC-CDR1 having the amino acid sequence of SEQ ID NO:41
   LC-CDR2 having the amino acid sequence of SEQ ID NO:67
   LC-CDR3 having the amino acid sequence of SEQ ID NO:43.

17. The antigen-binding molecule according to any one of paras 1 to 7, wherein the antigen-binding molecule comprises:
(i) a heavy chain variable (VH) region incorporating the following CDRs:
   HC-CDR1 having the amino acid sequence of SEQ ID NO:53
   HC-CDR2 having the amino acid sequence of SEQ ID NO:34
   HC-CDR3 having the amino acid sequence of SEQ ID NO:35; and
(ii) a light chain variable (VL) region incorporating the following CDRs:
   LC-CDR1 having the amino acid sequence of SEQ ID NO:41
   LC-CDR2 having the amino acid sequence of SEQ ID NO:58
   LC-CDR3 having the amino acid sequence of SEQ ID NO:43.

18. The antigen-binding molecule according to any one of paras 1 to 6, wherein the antigen-binding molecule comprises:
(i) a heavy chain variable (VH) region incorporating the following CDRs:
   HC-CDR1 having the amino acid sequence of SEQ ID NO:72
   HC-CDR2 having the amino acid sequence of SEQ ID NO:73
   HC-CDR3 having the amino acid sequence of SEQ ID NO:74; and
(ii) a light chain variable (VL) region incorporating the following CDRs:
   LC-CDR1 having the amino acid sequence of SEQ ID NO:80
   LC-CDR2 having the amino acid sequence of SEQ ID NO:81
   LC-CDR3 having the amino acid sequence of SEQ ID NO:82.

19. The antigen-binding molecule according to any one of paras 1 to 6, wherein the antigen-binding molecule comprises:
(i) a heavy chain variable (VH) region incorporating the following CDRs:
   HC-CDR1 having the amino acid sequence of SEQ ID NO:88
   HC-CDR2 having the amino acid sequence of SEQ ID NO:89
   HC-CDR3 having the amino acid sequence of SEQ ID NO:90; and
(ii) a light chain variable (VL) region incorporating the following CDRs:
   LC-CDR1 having the amino acid sequence of SEQ ID NO:96
   LC-CDR2 having the amino acid sequence of SEQ ID NO:97
   LC-CDR3 having the amino acid sequence of SEQ ID NO:98.

20. The antigen-binding molecule according to any one of paras 1 to 6, wherein the antigen-binding molecule comprises:
(i) a heavy chain variable (VH) region incorporating the following CDRs:
   HC-CDR1 having the amino acid sequence of SEQ ID NO:88
   HC-CDR2 having the amino acid sequence of SEQ ID NO:89
   HC-CDR3 having the amino acid sequence of SEQ ID NO:90; and
(ii) a light chain variable (VL) region incorporating the following CDRs:
   LC-CDR1 having the amino acid sequence of SEQ ID NO:137
   LC-CDR2 having the amino acid sequence of SEQ ID NO:138
   LC-CDR3 having the amino acid sequence of SEQ ID NO:139.

21. The antigen-binding molecule according to any one of paras 1 to 6, wherein the antigen-binding molecule comprises:
(i) a heavy chain variable (VH) region incorporating the following CDRs:
   HC-CDR1 having the amino acid sequence of SEQ ID NO:33
   HC-CDR2 having the amino acid sequence of SEQ ID NO:107
   HC-CDR3 having the amino acid sequence of SEQ ID NO:108,
or a variant thereof in which one or two or three amino acids in one or more of HC-CDR1, HC-CDR2, or HC-CDR3 are substituted with another amino acid; and
(ii) a light chain variable (VL) region incorporating the following CDRs:
   LC-CDR1 having the amino acid sequence of SEQ ID NO:114
   LC-CDR2 having the amino acid sequence of SEQ ID NO:67
   LC-CDR3 having the amino acid sequence of SEQ ID NO:115.

22. The antigen-binding molecule according to any one of paras 1 to 6, wherein the antigen-binding molecule comprises:
(i) a heavy chain variable (VH) region incorporating the following CDRs:
   HC-CDR1 having the amino acid sequence of SEQ ID NO:120
   HC-CDR2 having the amino acid sequence of SEQ ID NO:121
   HC-CDR3 having the amino acid sequence of SEQ ID NO:122; and
(ii) a light chain variable (VL) region incorporating the following CDRs:
   LC-CDR1 having the amino acid sequence of SEQ ID NO:127
   LC-CDR2 having the amino acid sequence of SEQ ID NO:128
   LC-CDR3 having the amino acid sequence of SEQ ID NO:129.

23. The antigen-binding molecule according to any one of paras 1 to 6, wherein the antigen-binding molecule comprises:
(i) a heavy chain variable (VH) region incorporating the following CDRs:
   HC-CDR1 having the amino acid sequence of SEQ ID NO:144
   HC-CDR2 having the amino acid sequence of SEQ ID NO:145
   HC-CDR3 having the amino acid sequence of SEQ ID NO:146; and
(ii) a light chain variable (VL) region incorporating the following CDRs:
   LC-CDR1 having the amino acid sequence of SEQ ID NO:151
   LC-CDR2 having the amino acid sequence of SEQ ID NO:152
   LC-CDR3 having the amino acid sequence of SEQ ID NO:153.

24. The antigen-binding molecule according to any one of paras 1 to 6, wherein the antigen-binding molecule comprises:
(i) a heavy chain variable (VH) region incorporating the following CDRs:
   HC-CDR1 having the amino acid sequence of SEQ ID NO:158
   HC-CDR2 having the amino acid sequence of SEQ ID NO:159
   HC-CDR3 having the amino acid sequence of SEQ ID NO:160; and
(ii) a light chain variable (VL) region incorporating the following CDRs:
   LC-CDR1 having the amino acid sequence of SEQ ID NO:165
   LC-CDR2 having the amino acid sequence of SEQ ID NO:152
   LC-CDR3 having the amino acid sequence of SEQ ID NO:153.

25. The antigen-binding molecule according to any one of paras 1 to 6, wherein the antigen-binding molecule comprises:
(i) a heavy chain variable (VH) region incorporating the following CDRs:
   HC-CDR1 having the amino acid sequence of SEQ ID NO:169
   HC-CDR2 having the amino acid sequence of SEQ ID NO:170
   HC-CDR3 having the amino acid sequence of SEQ ID NO:171; and
(ii) a light chain variable (VL) region incorporating the following CDRs:
   LC-CDR1 having the amino acid sequence of SEQ ID NO:177
   LC-CDR2 having the amino acid sequence of SEQ ID NO:178
   LC-CDR3 having the amino acid sequence of SEQ ID NO:179.

26. The antigen-binding molecule according to any one of paras 1 to 6, wherein the antigen-binding molecule comprises:
(i) a heavy chain variable (VH) region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:246; and
(ii) a light chain variable (VL) region incorporating the following CDRs:
   LC-CDR1 having the amino acid sequence of SEQ ID NO:247
   LC-CDR2 having the amino acid sequence of SEQ ID NO:178
   LC-CDR3 having the amino acid sequence of SEQ ID NO:190.

27. The antigen-binding molecule according to any one of paras 1 to 6 or para 26, wherein the antigen-binding molecule comprises:
(i) a heavy chain variable (VH) region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:185; and
(ii) a light chain variable (VL) region incorporating the following CDRs:
   LC-CDR1 having the amino acid sequence of SEQ ID NO:189
   LC-CDR2 having the amino acid sequence of SEQ ID NO:178
   LC-CDR3 having the amino acid sequence of SEQ ID NO:190.

28. The antigen-binding molecule according to any one of paras 1 to 6 or para 26, wherein the antigen-binding molecule comprises:
(i) a heavy chain variable (VH) region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:195; and
(ii) a light chain variable (VL) region incorporating the following CDRs:
   LC-CDR1 having the amino acid sequence of SEQ ID NO:197
   LC-CDR2 having the amino acid sequence of SEQ ID NO:178
   LC-CDR3 having the amino acid sequence of SEQ ID NO:190.

29. The antigen-binding molecule according to any one of paras 1 to 6 or para 26, wherein the antigen-binding molecule comprises:
(i) a heavy chain variable (VH) region incorporating the following CDRs:
   HC-CDR1 having the amino acid sequence of SEQ ID NO:72
   HC-CDR2 having the amino acid sequence of SEQ ID NO:184
   HC-CDR3 having the amino acid sequence of SEQ ID NO:200; and
(ii) a light chain variable (VL) region incorporating the following CDRs:
   LC-CDR1 having the amino acid sequence of SEQ ID NO:203
   LC-CDR2 having the amino acid sequence of SEQ ID NO:178
   LC-CDR3 having the amino acid sequence of SEQ ID NO:190.

30. The antigen-binding molecule according to any one of paras 1 to 6, wherein the antigen-binding molecule comprises:
a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:310;
   or
a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:294;
   or
a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:297;
   or
a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:299;
   or
a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:301;
   or
a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:302;
   or
a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:303;
   or
a VH region comprising an amino acid sequence having at least 70% sequence identity to the amino acid sequence of SEQ ID NO:276; and
a VL region comprising an amino acid sequence having at least 70% sequence identity to the amino acid sequence of SEQ ID NO:282;
   or
a VH region comprising an amino acid sequence having at least 70% sequence identity to the amino acid sequence of SEQ ID NO:285; and
a VL region comprising an amino acid sequence having at least 70% sequence identity to the amino acid sequence of SEQ ID NO:287;
a VH region comprising an amino acid sequence having at least 70% sequence identity to the amino acid sequence of SEQ ID NO:32; and
a VL region comprising an amino acid sequence having at least 70% sequence identity to the amino acid sequence of SEQ ID NO:40;
   or
a VH region comprising an amino acid sequence having at least 70% sequence identity to the amino acid sequence of SEQ ID NO:52; and
a VL region comprising an amino acid sequence having at least 70% sequence identity to the amino acid sequence of SEQ ID NO:57;
   or
a VH region comprising an amino acid sequence having at least 70% sequence identity to the amino acid sequence of SEQ ID NO:62; and
a VL region comprising an amino acid sequence having at least 70% sequence identity to the amino acid sequence of SEQ ID NO:66;
   or
a VH region comprising an amino acid sequence having at least 70% sequence identity to the amino acid sequence of SEQ ID NO:48; and
a VL region comprising an amino acid sequence having at least 70% sequence identity to the amino acid sequence of SEQ ID NO:50;
   or.
a VH region comprising an amino acid sequence having at least 70% sequence identity to the amino acid sequence of SEQ ID NO:87; and
a VL region comprising an amino acid sequence having at least 70% sequence identity to the amino acid sequence of SEQ ID NO:95;
   or
a VH region comprising an amino acid sequence having at least 70% sequence identity to the amino acid sequence of SEQ ID NO:106; and
a VL region comprising an amino acid sequence having at least 70% sequence identity to the amino acid sequence of SEQ ID NO:113;
   or
a VH region comprising an amino acid sequence having at least 70% sequence identity to the amino acid sequence of SEQ ID NO:143; and
a VL region comprising an amino acid sequence having at least 70% sequence identity to the amino acid sequence of SEQ ID NO:150;
   or
a VH region comprising an amino acid sequence having at least 70% sequence identity to the amino acid sequence of SEQ ID NO:157; and
a VL region comprising an amino acid sequence having at least 70% sequence identity to the amino acid sequence of SEQ ID NO:164;
   or
a VH region comprising an amino acid sequence having at least 70% sequence identity to the amino acid sequence of SEQ ID NO:71; and
a VL region comprising an amino acid sequence having at least 70% sequence identity to the amino acid sequence of SEQ ID NO:79;
   or
a VH region comprising an amino acid sequence having at least 70% sequence identity to the amino acid sequence of SEQ ID NO:102; and
a VL region comprising an amino acid sequence having at least 70% sequence identity to the amino acid sequence of SEQ ID NO:104;
   or
a VH region comprising an amino acid sequence having at least 70% sequence identity to the amino acid sequence of SEQ ID NO:119; and
a VL region comprising an amino acid sequence having at least 70% sequence identity to the amino acid sequence of SEQ ID NO:126;
   or
a VH region comprising an amino acid sequence having at least 70% sequence identity to the amino acid sequence of SEQ ID NO:183; and
a VL region comprising an amino acid sequence having at least 70% sequence identity to the amino acid sequence of SEQ ID NO:188;
   or
a VH region comprising an amino acid sequence having at least 70% sequence identity to the amino acid sequence of SEQ ID NO:194; and
a VL region comprising an amino acid sequence having at least 70% sequence identity to the amino acid sequence of SEQ ID NO:196;
   or
a VH region comprising an amino acid sequence having at least 70% sequence identity to the amino acid sequence of SEQ ID NO:199; and
a VL region comprising an amino acid sequence having at least 70% sequence identity to the amino acid sequence of SEQ ID NO:202;
   or
a VH region comprising an amino acid sequence having at least 70% sequence identity to the amino acid sequence of SEQ ID NO:133; and
a VL region comprising an amino acid sequence having at least 70% sequence identity to the amino acid sequence of SEQ ID NO:136;
   or
a VH region comprising an amino acid sequence having at least 70% sequence identity to the amino acid sequence of SEQ ID NO:168; and
a VL region comprising an amino acid sequence having at least 70% sequence identity to the amino acid sequence of SEQ ID NO:176.

31. The antigen-binding molecule according to any one of paras 1 to 30, wherein the antigen-binding molecule is capable of binding to human VISTA and one or more of: mouse VISTA and cynomolgus macaque VISTA.

32. An antigen-binding molecule, optionally isolated, comprising (i) an antigen-binding molecule according to any one of paras 1 to 31, and (ii) an antigen-binding molecule capable of binding to an antigen other than VISTA.

33. The antigen-binding molecule according to any one of paras 1 to 32, wherein the antigen-binding molecule is capable of binding to cells expressing VISTA at the cell surface.

34. The antigen-binding molecule according to any one of paras 1 to 33, wherein the antigen-binding molecule is capable of inhibiting interaction between VISTA and a binding partner for VISTA.

35. The antigen-binding molecule according to any one of paras 1 to 34, wherein the antigen-binding molecule is capable of inhibiting VISTA-mediated signalling.

36. The antigen-binding molecule according to any one of paras 1 to 35, wherein the antigen-binding molecule is capable of increasing proliferation and/or cytokine production by effector immune cells.

37. A chimeric antigen receptor (CAR) comprising an antigen-binding molecule according to any one of paras 1 to 36.

38. A nucleic acid, or a plurality of nucleic acids, optionally isolated, encoding an antigen-binding molecule according to any one of paras 1 to 36 or a CAR according to para 37.

39. An expression vector, or a plurality of expression vectors, comprising a nucleic acid or a plurality of nucleic acids according to para 38.

40. A cell comprising an antigen-binding molecule according to any one of paras 1 to 36, a CAR according to para 37, a nucleic acid or a plurality of nucleic acids according to para 38, or an expression vector or a plurality of expression vectors according to para 39.

41. A method comprising culturing a cell comprising a nucleic acid or a plurality of nucleic acids according to para 38, or an expression vector or a plurality of expression vectors according to para 39, under conditions suitable for expression of the antigen-binding molecule or CAR from the nucleic acid(s) or expression vector(s).

42. A composition comprising an antigen-binding molecule according to any one of paras 1 to 36, a CAR according to para 37, a nucleic acid or a plurality of nucleic acids according to para 38, an expression vector or a plurality of expression vectors according to para 39, or a cell according to para 40.

43. The composition according to para 42, additionally comprising an agent capable of inhibiting signalling mediated by an immune checkpoint molecule other than VISTA, optionally wherein the immune checkpoint molecule other than VISTA is selected from PD-1, CTLA-4, LAG-3, TIM-3, TIGIT and BTLA.

44. An antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43 for use in a method of medical treatment or prophylaxis.

45. An antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43, for use in a method of treatment or prevention of a cancer or an infectious disease.

46. Use of an antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43, in the manufacture of a medicament for use in a method of treatment or prevention of a cancer or an infectious disease.

47. A method of treating or preventing a cancer or an infectious disease, comprising administering to a subject a therapeutically or prophylactically effective amount of an antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43.

48. The antigen-binding molecule, CAR, nucleic acid or plurality of nucleic acids, expression vector or plurality of expression vectors, cell or composition for use according to para 45, the use according to para 46 or the method according to para 47, wherein the cancer is selected from: colorectal cancer, pancreatic cancer, breast cancer, liver cancer, prostate cancer, ovarian cancer, head and neck cancer, leukemia, lymphoma, melanoma, thymoma, lung cancer, non-small cell lung cancer (NSCLC) and a solid tumor.

49. An antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43, for use in a method of treatment or prevention of a disease in which myeloid-derived suppressor cells (MDSCs) are pathologically implicated.

50. Use of an antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43, in the manufacture of a medicament for use in a method of treatment or prevention of a disease in which myeloid-derived suppressor cells (MDSCs) are pathologically implicated.

51. A method of treating or preventing a disease in which myeloid-derived suppressor cells (MDSCs) are pathologically implicated, comprising administering to a subject a therapeutically or prophylactically effective amount of an antigen-binding molecule according to any one of paras 1 to 36, a CAR according to para 37, a nucleic acid or a plurality of nucleic acids according to para 38, an expression vector or a plurality of expression vectors according to para 39, a cell according to para 40, or a composition according to para 42 or para 43.

52. The antigen-binding molecule, CAR, nucleic acid or plurality of nucleic acids, expression vector or plurality of expression vectors, cell or composition for use, the use, or the method according to any one of paras 45 to 51, wherein the method additionally comprises administration of an agent capable of inhibiting signalling mediated by an immune checkpoint molecule other than VISTA, optionally wherein the immune checkpoint molecule other than VISTA is selected from PD-1, CTLA-4, LAG-3, TIM-3, TIGIT or BTLA.

53. A method of inhibiting VISTA-mediated signalling, comprising contacting VISTA-expressing cells with an antigen-binding molecule according to any one of paras 1 to 36.

54. A method for inhibiting the activity of myeloid-derived suppressor cells (MDSCs), the method comprising contacting MDSCs with an antigen-binding molecule according to any one of paras 1 to 36.

55. A method for increasing the number or activity of effector immune cells, the method comprising inhibiting the activity of VISTA-expressing cells with an antigen-binding molecule according to any one of paras 1 to 36.

56. An in vitro complex, optionally isolated, comprising an antigen-binding molecule according to any one of paras 1 to 36 bound to VISTA.

57. A method comprising contacting a sample containing, or suspected to contain, VISTA with an antigen-binding molecule according to any one of paras 1 to 36, and detecting the formation of a complex of the antigen-binding molecule with VISTA.

58. A method of selecting or stratifying a subject for treatment with a VISTA-targeted agent, the method comprising contacting, in vitro, a sample from the subject with an antigen-binding molecule according to any one of paras 1 to 36 and detecting the formation of a complex of the antigen-binding molecule with VISTA.

59. Use of an antigen-binding molecule according to any one of paras 1 to 36 as an in vitro or in vivo diagnostic or prognostic agent.

60. Use of an antigen-binding molecule according to any one of paras 1 to 36 in a method for detecting, localizing or imaging a cancer, optionally wherein the cancer is selected from: colorectal cancer, pancreatic cancer, breast cancer, liver cancer, prostate cancer, ovarian cancer, head and neck cancer, leukemia, lymphoma, melanoma, thymoma, lung cancer, non-small cell lung cancer (NSCLC) and a solid tumor.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed herein, the reverse complement thereof is also expressly contemplated.

Methods described herein may preferably performed in vitro. The term “in vitro” is intended to encompass procedures performed with cells in culture whereas the term “in vivo” is intended to encompass procedures with/on intact multi-cellular organisms.

### BRIEF DESCRIPTION OF THE FIGURES

Embodiments and experiments illustrating the principles of the invention will now be discussed with reference to the accompanying figures.

FIGS. 1A to 1D. Histograms showing staining of cells by anti-VISTA antibodies as determined by flow cytometry. Histograms show staining of HEK293 cells (which do not express VISTA), or HEK293 VISTA overexpressing cells (HEK293 VISTA O/E) by anti-VISTA antibody clone (1A) VSTB112 (positive control; WO 2015/097536), (1B) 4-M2-D5, (1C) 9M2-C12 or (1D) 4M2-C12 (also referred to herein as “V4”).

FIG. 2. Histograms showing staining of cells by anti-VISTA antibodies as determined by flow cytometry. Histograms show staining of HEK293 cells (which do not express VISTA), or HEK293 VISTA overexpressing cells (HEK293 VISTA O/E) by anti-VISTA antibody clones 9M2C12, V4 and clone VSTB112, or an isotype control antibody. Unstained cells were analysed as a negative control.

FIGS. 3A to 3C. Sensorgrams showing the results of analysis of affinity of binding to human, cynomolgus monkey and murine VISTA by anti-VISTA antibody clone V4. (3A) shows binding to human VISTA, (3B) shows binding to cynomolgus monkey VISTA, and (3C) shows binding to murine VISTA. Kon, Koff and K_{D} are shown.

FIGS. 4A to 4B. Graphs showing the results of analysis of binding of anti-VISTA antibodies to different proteins. 4A shows binding to human, cynomolgus monkey and murine VISTA, human PD-L1 and human HER3 by anti-VISTA antibody clone V4, as determined by ELISA. 4B shows binding of anti-VISTA antibody clone V4 to human VISTA, PD-1, PD-L1, B7H3, B7H4, B7H6, B7H7, CTLA4 and an irrelevant antigen.

FIG. 5. Sensorgrams showing the results of analysis of binding between VISTA and VSIG-3.

FIG. 6. Graph showing the results of analysis of inhibition of binding between VISTA and VSIG-3 by anti-VISTA antibody clones 5M1-A11 and 9M2-C12.

FIGS. 7A and 7B. Graph and bar chart showing results of the analysis of the effect of treatment with anti-VISTA antibody clone 13D5p on production of IFN-γ, IL-2 and IL-17A in a mixed lymphocyte reaction (MLR) assay. (7A) shows the level of cytokine detected in the cell culture supernatant at the end of the assay, and (7B) shows the fold-change (“FC”) in the level of the indicated cytokines.

FIGS. 8A and 8B. Graph and bar chart showing results of the analysis of the effect of treatment with anti-PD-L1 antibody clone MIH5 (ThermoFisher Scientific) on production of IFN-γ, IL-2 and IL-17A in a mixed lymphocyte reaction (MLR) assay. (8A) shows the level of cytokine detected in the cell culture supernatant at the end of the assay, and (8B) shows the fold-change (“FC”) in the level of the indicated cytokines.

FIG. 9. Graph showing the results of analysis of stability of anti-VISTA antibody clone V4 by Differential Scanning Fluorimetry analysis.

FIG. 10. Graph showing the results of the analysis of anti-VISTA antibody clone V4 by size exclusion chromatography.

FIG. 11. Images showing the results of the analysis of anti-VISTA antibody clone V4 expression by SDS-PAGE and western blot. Lanes: M1=TaKaRa protein marker Cat. No. 3452; M2=GenScript protein marker Cat. No. M00521; 1=reducing conditions; 2=non-reducing conditions; P=positive control: mouse IgG1, Kappa (Sigma Cat. No. M9269). For western blot, the primary antibody used was goat anti-mouse IgG (H+L) antibody (LI-COR, Cat. No. 926-32210).

FIG. 12. Graph and table showing the results of the pharmacokinetics analysis of anti-VISTA antibody clone V4 by ELISA analysis of antibody serum.

FIG. 13. Graph showing the results of the analysis of anti-cancer activity of anti-VISTA antibody clone V4 in vivo in a syngeneic cell-line derived mouse model of colon carcinoma.

FIG. 14. Bar chart showing inhibition of tumor growth at day 15 in a syngeneic cell-line derived mouse model of colon carcinoma following treatment with monotherapy or combination therapy targeting the indicated checkpoint molecules. The antibodies used in this study were as follows: anti-VISTA=clone V4, anti-PD-L1=clone 10F.9G2, anti-TIGIT=clone 1G9, anti-LAG-3=clone C9B7W, and anti-TIM-3=clone RMT3-23.

FIG. 15. Bar chart showing the number of MDSCs per 100,000 cells, and the ratio of CD8+ T cell: Tregs, in the tumor bulk of a syngeneic cell-line derived mouse model of colon carcinoma, as determined by RNA-Seq analysis of bulk tumor following treatment with anti-VISTA antibody clone V4 alone, anti-PD-L1 antibody clone 10F.9G2 alone, combination treatment with anti-VISTA antibody clone V4 and anti-PD-L1 antibody clone 10F.9G2, or treatment with PBS (negative control).

FIG. 16. Graph showing the results of the analysis of anti-cancer activity of anti-VISTA antibody clone V4 in vivo in a syngeneic cell-line derived mouse model of Lewis lung carcinoma.

FIG. 17. Graph showing the results of the analysis of anti-cancer activity of anti-VISTA antibody clone V4 in vivo in a syngeneic cell-line derived mouse model of melanoma, as a monotherapy, or in combination with an anti-PD-1 antibody RMP1-14 (Bio X Cell).

FIG. 18. Bar chart showing numbers or different types of white blood cells after administration of a single dose of 900 μg of anti-VISTA antibody clone V4, or an equal volume of vehicle (PBS) as a negative control.

FIGS. 19A and 19B. Bar charts showing analysis of hepatotoxicity and nephrotoxicity, by evaluation of correlates of (19A) liver and (19B) kidney function following administration of a single dose of 900 μg of anti-VISTA antibody clone V4, or an equal volume of vehicle (PBS) as a negative control. (19A) shows levels of alanine aminotransferase (ALT) and aspartate transaminase (AST), and (19B) shows level of blood urea nitrogen (BUN) and creatinine (CREA).

FIG. 20. Graph showing the results of analysis of binding to human, cynomolgus monkey and mouse VISTA and human PD-L1 by anti-VISTA antibody clone 13D5-1, as determined by ELISA.

FIG. 21. Graph showing the results of analysis of binding to human and mouse VISTA by anti-VISTA antibody clone 13D5-13, as determined by ELISA.

FIG. 22. Graph showing the results of the analysis of anti-cancer activity of anti-VISTA antibody clone 13D5-1 in vivo in a cell-line derived mouse model of colon carcinoma, alone or in combination with anti-PD-L1.

FIG. 23. Graph showing the results of the analysis of anti-cancer activity of anti-VISTA antibody clone 13D5-1 in vivo in a cell-line derived mouse model of mammary carcinoma.

FIG. 24. Histograms showing staining of cells by anti-VISTA antibodies as determined by flow cytometry. Histograms show staining of HEK293 cells (which do not express VISTA), or HEK293 VISTA overexpressing cells (HEK293 VISTA O/E) by anti-VISTA antibody clones 4M2-B4, 2M1-B12, 4M2-C9, 2M1-D2, 4M2-D9, 1M2-D2, 5M1-A11, 4M2-D5, 4M2-A8 and 9M2-C12.

FIGS. 25A to 25D. Sensorgrams showing the results of analysis of binding of 4M2-C12 mIgG1 to (25A) mouse FcγRIV, (25B) mouse FcγRIII, (25C) mouse FcγRIIb, and (25D) mouse FcRn.

FIGS. 26A to 26D. Sensorgrams showing the results of analysis of binding of 4M2-C12 mIgG2a to (26A) mouse FcγRIV, (26B) mouse FcγRIII, (26C) mouse FcγRIIb, and (26D) mouse FcRn.

FIGS. 27A to 27D. Sensorgrams showing the results of analysis of binding of 4M2-C12 mIgG2a LALA PG to (27A) mouse FcγRIV, (27B) mouse FcγRIII, (27C) mouse FcγRIIb, and (27D) mouse FcRn.

FIGS. 28A to 28D. Sensorgrams showing the results of analysis of binding of 4M2-C12 mIgG2a NQ to (28A) mouse FcγRIV, (28B) mouse FcγRIII, (28C) mouse FcγRIIb, and (28D) mouse FcRn.

FIGS. 29A to 29C. Tables summarising the results of analysis of binding of 4M2-C12 mIgG1, 4M2-C12 mIgG2a, 4M2-C12 mIgG2a LALA PG and 4M2-C12 mIgG2a to mouse FcγRIV, mouse FcγRIII, mouse FcγRIIb, and mouse FcRn. 29A shows calculated Kₒₙ values, 29B shows calculated K_{dis} values and 29C shows calculated K_{D} values.

FIGS. 30A to 30C. Graphs showing the results of the analysis of anti-cancer activity in vivo of anti-VISTA antibody clone 4M2-C12 in mIgG2a and mIgG2a LALA PG formats, in a cell-line derived mouse model of T cell lymphoma. 30A shows data for the different treatment groups, 30B shows the data obtained for individual mice in the vehicle control and 4M2-C12 mIgG2a treatment groups, and 30C shows the data obtained for individual mice in the vehicle control and 4M2-C12 mIgG2a LALA PG treatment groups.

FIGS. 31A and 31B. Sensorgram showing the results of analysis of competition between different anti-VISTA antibodies for binding to human VISTA by BLI.

FIG. 32. Graph showing the results of analysis of inhibition of binding between VISTA and VSIG-3 by anti-VISTA antibody 4M2-C12.

FIGS. 33A to 33D. Bar charts showing the results of analysis of the ability of anti-VISTA antibodies to restore T cell proliferation to T cells treated with VISTA-Ig, as determined by CFSE dilution assay. FIGS. 33A and 33C show results obtained from conditions using wells coated with a 1:1 ratio of agonist anti-CD3 antibody and VISTA-Ig, and FIGS. 33B and 33D show results obtained from conditions using wells coated with a 2:1 ratio of agonist anti-CD3 antibody and VISTA-Ig. FIGS. 33A and 33B show the percentages of CFSE-low CD4+ T cells, and FIGS. 33C and 33D show the percentages of CFSE-low CD8+ T cells.

FIGS. 34A and 34B. Bar charts showing the results of analysis of the ability of anti-VISTA antibodies to promote the production of IL-6 by LPS-stimulated THP-1 cells. FIG. 34A shows the results obtained using 4M2-C12, and FIG. 34B shows the results obtained using VSTB112.

FIG. 35. Bar chart showing the level of IL-6 detected in blood samples obtained from mice administered with anti-VISTA antibody 4M2-C12, at 2h prior to administration, and at 0.5 hr, 6 hr, 24 hr and 96 hr after administration.

FIGS. 36A and 36B. Graphs showing the results of the analysis of anti-cancer activity in vivo of anti-VISTA antibody 4M2-C12 mIgG2a (V4), anti-PD-1 antibody (PD1) or combination treatment with 4M2-C12 mIgG2a and anti-PD-1 antibody, in a cell-line derived mouse model of colon carcinoma. 36A shows data for the different treatment groups, 36B shows the data obtained for individual mice in the vehicle control and 4M2-C12 mIgG2a+anti-PD-1 treatment groups.

FIG. 37. Bar chart showing the percentage of tumor-infiltrating CD45+ cells which are g-MDSCs of day 22 tumors of a cell-line derived mouse model of colon carcinoma, obtained from mice treated with PBS (Vehicle), anti-VISTA antibody 4M2-C12 mIgG2a (V4), anti-PD-1 antibody (Anti-PD1) or combination treatment with 4M2-C12 mIgG2a and anti-PD-1 antibody (Combo).

FIGS. 38A to 38E. Bar charts showing levels of (38A) IFNγ, (38B) IL-23, (38C) IL-10, (38D) IL-4, and (38E) IL-5 in serum obtained at day 18 of a cell-line derived mouse model of colon carcinoma, obtained from mice treated with PBS (Vehicle), anti-VISTA antibody 4M2-C12 mIgG2a (V4), anti-PD-1 antibody (PD1) or combination treatment with 4M2-C12 mIgG2a and anti-PD-1 antibody (V4+PD1).

FIG. 39. Bar chart showing the level of Arg1 RNA expression in tumors at day 21 of a cell-line derived mouse model of colon carcinoma, obtained from mice treated with PBS (Vehicle), anti-VISTA antibody 4M2-C12 mIgG2a (V4), anti-PD-L1 antibody (PDL1) or combination treatment with 4M2-C12 mIgG2a and anti-PD-1 antibody (V4+PDL1).

FIGS. 40A and 40B. Graphs showing the results of the analysis of anti-cancer activity in vivo of anti-VISTA antibody 4M2-C12 mIgG2a (V4), anti-PD-1 antibody (PD1) or combination treatment with 4M2-C12 mIgG2a and anti-PD-1 antibody, in a cell-line derived mouse model of melanoma. 40A shows data for the different treatment groups, 40B shows the data obtained for individual mice in the vehicle control and 4M2-C12 mIgG2a+anti-PD-1 treatment groups.

FIG. 41. Bar chart showing the percentage of tumor-infiltrating CD45+ cells which are g-MDSCs of day 18 tumors of a cell-line derived mouse model of melanoma, obtained from mice treated with PBS (Vehicle), anti-VISTA antibody 4M2-C12 mIgG2a (V4), anti-PD-1 antibody (Anti-PD1) or combination treatment with 4M2-C12 mIgG2a and anti-PD-1 antibody (Combo).

FIG. 42. Graph showing the results of the analysis of anti-cancer activity in vivo of anti-VISTA antibody 4M2-C12 mIgG2a (V4), anti-PD-1 antibody (Anti-PD1) or combination treatment with 4M2-C12 mIgG2a and anti-PD-1 antibody (V4+Anti-PD1), in a cell-line derived mouse model of T cell leukemia/lymphoma.

FIG. 43. Bar chart showing the percentage of tumor-infiltrating CD45+ cells which are g-MDSCs of day 16 tumors of a cell-line derived mouse model of T cell leukemia/lymphoma, obtained from mice treated with PBS (Vehicle), anti-VISTA antibody 4M2-C12 mIgG2a (V4), anti-PD-1 antibody (Anti-PD1) or combination treatment with 4M2-C12 mIgG2a and anti-PD-1 antibody (Combo).

FIG. 44. Graph showing the weights of mice during the course of treatment of a cell-line derived mouse model of colon carcinoma with PBS (Vehicle), anti-VISTA antibody 4M2-C12 mIgG2a (V4), anti-PD-L1 antibody (Anti-PDL1) or combination treatment with 4M2-C12 mIgG2a and anti-PD-1 antibody (V4+Anti-PDL1).

FIGS. 45A to 45D. Sensorgrams and table showing the results of analysis of binding of different anti-VISTA antibodies to human VISTA (45A) mouse VISTA (45B) and human PD-L1 (45C), as determined by Biolayer Interferometry. 45D summarises the kinetic and thermodynamic constants calculated from the sensorgrams of 45A and 45B.

FIGS. 46A and 46B. Sensorgrams and table showing the results of analysis of binding of different anti-VISTA antibodies to mouse VISTA, as determined by Biolayer Interferometry. 45B summarises the kinetic and thermodynamic constants calculated from the sensorgrams of 46A.

FIG. 47A to 47C. Sensorgrams and table showing the results of analysis of binding of different anti-VISTA antibodies to human VISTA (47A) and mouse VISTA (47B), as determined by Biolayer

Interferometry. 47C summarises the kinetic and thermodynamic constants calculated from the sensorgrams of 47A and 47B.

FIGS. 48A and 48B. Sensorgrams and table showing the results of analysis of binding of different anti-VISTA antibodies to human VISTA, and mouse VISTA and human CD47, as determined by Biolayer Interferometry. 48B summarises the kinetic and thermodynamic constants calculated from the sensorgrams of 48A.

FIGS. 49A to 49C. Concentration-response graphs and table showing the results of analysis of binding of different antibodies to human VISTA (49A) or mouse VISTA (49B), as determined by ELISA. 49C shows EC₅₀ values (nM) for binding of the different antibodies to the indicated proteins.

FIGS. 50A to 50C. Concentration-response graphs and table showing the results of analysis of binding of different antibodies to human VISTA (50A) and mouse VISTA (50B), as determined by ELISA. 50C shows EC₅₀ values (nM) for binding of the different antibodies to the indicated proteins.

FIGS. 51A to 51C. Concentration-response graphs and table showing the results of analysis of binding of different antibodies to human VISTA (51A) and mouse VISTA (51B), as determined by ELISA. 51C shows EC₅₀ values (nM) for binding of the different antibodies to the indicated proteins.

FIGS. 52A to 52J. Melting graphs and table showing the results of analysis of stability of different anti-VISTA antibodies by Differential Scanning Fluorimetry analysis. 52A to 52I show the first derivate of the raw data obtained for test antibody preparations and no protein control (NPC) preparations, in triplicate. 52J summarises the results of 52A to 52I.

FIG. 53. Table summarising the results of in silico analysis of different anti-VISTA antibodies for safety and immunogenicity.

FIG. 54. Graph showing the results of analysis of inhibition of binding between VISTA and VSIG-3 by anti-VISTA antibody 4M2-C12.

FIG. 55. Bar chart showing the results of analysis of inhibition of binding between VISTA and PSGL-1 by anti-VISTA antibody clone 4M2-C12 (V4).

FIGS. 56A to 56G. Concentration-response graphs showing the results of analysis of binding of (56A) V4, (56B) V4-C24, (56C) V4-C26, (56D) V4-C27, (56E) V4-C28, (56F) V4-C30 and (56G) V4-C31 to human VISTA, PD-L1, B7H3, B7H4, B7H6, B7H7, PD-1 and CTLA-4, as determined by ELISA.

FIGS. 57A to 57I. Concentration-response graphs showing the results of analysis of binding of (57A) V4, (57B) V4-C24, (57C) V4-C26, (57D) V4-C27, (57E) V4-C28, (57F) V4-C30 (57G) V4-C31 and (57H) isotype-matched control antibody to human VISTA, mouse VISTA, rat VISTA and cyno VISTA, as determined by ELISA. 57I shows EC₅₀ values (M) for binding of the different antibodies to the indicated proteins.

FIGS. 58A to 58C. Histograms showing staining of cells by different anti-VISTA antibodies or isotype control antibody as determined by flow cytometry. 58A shows binding of antibodies to wildtype, non-transfected HEK293-6E cells. 58B shows binding of antibodies to HEK293-6E cells overexpressing human VISTA protein. 58C shows binding of antibodies to HEK293-6E cells overexpressing mouse VISTA protein. 1=no antibody (unstained), 2=human IgG1 Isotype control antibody, 3=VSTB112 IgG1, 4=4M2-C12 IgG1, 5=V4-C24 IgG1, 6=V4-C26 IgG1, 7=V4-C27 IgG1, 8=V4-C28 IgG1, 9=V4-C30 IgG1, and 10=V4-C31 IgG1.

FIGS. 59A and 59B. Images showing immunohistochemical staining of human tissue using anti-VISTA antibody. 59A shows staining of normal human spleen tissue by 4M2-C12 mIgG2a, and 59B shows staining of normal human ovary tissue by 4M2-C12 mIgG2a, at the indicated magnifications.

FIGS. 60A to 60D. Histogram and bar charts showing the results of analysis of the ability of anti-VISTA antibodies 4M2-C12-hIgG1 (V4) or VSTB112 to release activated T cells from suppression by VISTA-expressing cells. 60A and 60B show the results of CFSE dilution analysis of T cell proliferation in the presence of VISTA-expressing cells and the indicated quantities of anti-VISTA antibodies, for 5 days. 60C and 60D show the concentration of IFNγ (60C) and TNFa (60D) detected in the cell culture supernatant after 5 days.

FIGS. 61A to 61C. Bar charts showing the results of analysis of the ability of anti-VISTA antibodies 4M2-C12-hIgG1 (V4) or VSTB112 to promote the production of cytokines by LPS-stimulated THP-1 cells. 61A shows the concentration of IL-6 detected in the cell culture supernatant, and 61B shows the concentration of TNFa detected in the cell culture supernatant of cells for 24 hours in the presence of the indicated quantities of anti-VISTA antibodies. 61C shows that the THP1 cells are VISTA-expressing cells; the percentage of the cells in culture determined to express VISTA is shown. 1=unstained, 2=cells stained with isotype-matched control antibody, 3=cells stained with 20 μg V4, 4=cells stained with 40 ug V4, and 5=cells stained with 20 μg VSTB112.

FIG. 62. Bar chart showing the results of analysis of the ability of anti-VISTA antibodies 4M2-C12-hIgG1 or 4M2-C12-hIgG4 to promote the production of IL-6 by LPS-stimulated THP-1 cells. The concentration of IL-6 detected in the cell culture supernatant is shown.

FIGS. 63A to 63D. Graphs and tables showing the results of the pharmacokinetics analysis of anti-VISTA antibodies 4M2-C12-hIgG1 and 4M2-C12-hIgG4 by ELISA analysis of antibody serum. Results are shown following administration of (63A) 10 mg/kg, (63B) 25 mg/kg, (63C) 100 mg/kg, or (63D) 250 mg/kg of antibody.

FIGS. 64A to 64C. Tables showing representative hematological profiles in BALB/C mice 96 hours after administration of 50 mg/kg 4M2-C12-hIgG1 or an equal volume of PBS. 64A shows results of analysis of the red blood cell compartment, 64B shows results of analysis of the white blood cell compartment, and 64C shows results of analysis of correlates of liver and kidney function. RBC=red blood cell, MVC=mean corpuscular volume, MCH=mean corpuscular haemoglobin, MCHC=mean corpuscular haemoglobin concentration, WBC=white blood cell, ALT=alanine aminotransferase, ALP=alkaline phosphatase, CREA=creatinine, and BUN=blood urea nitrogen.

FIGS. 65A to 65C. Tables showing representative hematological profiles of SD rats following administration of 250 mg/kg 4M2-C12-hIgG1, 250 mg/kg 4M2-C12-hIgG4 or an equal volume of PBS. 65A shows results of analysis of the red blood cell compartment, 65B shows results of analysis of the white blood cell compartment, and 65C shows results of analysis of correlates of liver, kidney and pancreas function, and levels of electrolytes. RBC=red blood cell, MVC=mean corpuscular volume, MCH=mean corpuscular haemoglobin, MCHC=mean corpuscular haemoglobin concentration, WBC=white blood cell, ALT=alanine aminotransferase, ALP=alkaline phosphatase, CREA=creatinine, BUN=blood urea nitrogen, GLU=glucagon, AMY=amylase, NA=sodium, K=potassium, P=phosphorus and CA=calcium.

FIG. 66. Sensorgrams showing the background-subtracted binding signal for anti-VISTA antibodies V4-C26, 13F3 and VSTB112 to human VISTA and mouse VISTA as determined by Bio-Layer Interferometry.

FIGS. 67A and 67B. Graphs showing the effects of administration of V4-C26.hIgG4 on tumour volume and survival of mice in a CT26 cell-derived mouse model of colon carcinoma. (67A) shows tumour volume over time, and (67B) shows percent survival over time for mice administered with anti-VISTA antibody V4-C26 hIgG4 or vehicle control.

FIGS. 68A to 68H. Scatterplots and images showing VISTA expression by cells in healthy tissues and in certain cancers, and VSIG3 expression in certain cancers. (68A) Immune cell cluster identification by RNA single-cell analysis of 10× Genomics data comprising 68,000 healthy PBMCs. (68B, 68C) UMAP and summary plot of single-cell RNA-seq data for VISTA expression in different cell clusters, where cDC refers to Classical Dendritic Cells, pDC to Plasmacytoid Dendritic Cells and HSPC to Hematopoietic Stem and Progenitor Cells. (68D) Representative immunohistochemical staining for healthy spleen, bone marrow, breast and lung TMAs (n=99) stained with 4M2-C12-mIgG2a at 0.02 mg/ml; magnification, 200×. (68E) Representative staining for VISTA in triple negative breast cancer (TNBC; n=126) and non-small cell lung cancer (NSCLC; n=140) TMAs stained with 4M2-C12-mIgG2a at 0.02 mg/ml; magnification, 200×, and (68F) % of TNBC, NSCLC, hepato-cellular carcinoma (HCC) and mesothelioma patients with negative (0), low (1), moderate (2) or high (3) staining intensity for VISTA. (68G) Representative staining for VSIG3 in TNBC (n=126) and NSCLC (n=140) TMAs stained with anti-VSIG3 antibody at 0.003 mg/ml; magnification, 200×, and (68H) % of TNBC, NSCLC, HCC and mesothelioma patients with negative (0), low (1), moderate (2) or high (3) staining intensity for VSIG3.

FIGS. 69A and 69B. Graphs showing binding affinity of V4-C26 hIgG4 (HMBD-002) to (69A) FcγRIII and (69B) C1q proteins as assessed by ELISA. Data shown are mean of n=3 measurements and error bars are SEM.

FIGS. 70A to 70D. Graphs showing binding specificity of V4-C26 hIgG4. (70A) Binding of V4-C26 hIgG4 to human B7 family antigens, PD-1 and CTLA-4 as indicated, as determined by ELISA (data shown are mean n=2 measurements and error bars are SEM). (70B) Binding of V4-C26 hIgG4 to human, non-human primate (NHP), rat or mouse VISTA orthologs, as determined by ELISA (data shown are mean of n=3 measurements and error bars are SEM). (70C) Binding of V4-C26 hIgG4 to HEK293T cells overexpressing human, non-human primate (NHP), rat or mouse VISTA orthologs, as determined by flow cytometry (data shown are mean of n=3 measurements and error bars are SEM). (70D) Binding of V4-C26 hIgG4 to CD11b+ myeloid cells isolated from human, NHP, rat or mouse PBMCs (data shown are mean of n=3 measurements and error bars are SEM).

FIGS. 71A and 71B. Graphs showing VISTA blockade on myeloid cells by V4-C26 hIgG4. (71A) Anti-CD3 antibody-induced IFN-γ secretion from MDSCs co-cultured with autologous PBMCs after 96 hrs, in the presence or absence of V4-C26 hIgG4 (HMBD-002), VSTB112 or IgG4 isotype control, as measured by ELISA (data shown are mean of n=6 measurements and error bars are SEM and P-value was obtained by two-way ANOVA (Tukey's multiple comparison test), **** p<0.0001). (71B) Inhibition of neutrophil migration to the bottom chambers of transwell coated with C5a and measured using CellTiter-Glo (data shown are mean of n=3 measurements and error bars are SEM).

FIGS. 72A to 72D. Analysis of effect of VISTA blockade by V4-C26 hIgG4 on immune activation. (72A) Levels of the indicated cytokines measured at 96 hrs by Luminex, from the supernatant of an allogenic mixed lymphocyte reaction. Concentrations of V4-C26 hIgG4 and anti PD-1 antibody, Pembrolizumab (annotated as Pembro) are shown in ug/ml. Data were normalized to isotype control. Data shown are mean of n=10 and error bars are SEM. P-values were obtained by one-way ANOVA (Tukey's multiple comparison test), *p<0.05, *** p<0.001, **** p<0.0001. (72B) Speed2 pathway analysis from bulk RNA-sequencing of MLR samples (n=10). Data was normalized to isotype control, represented as Mean+SD and the p-adjusted values were obtained from bulk RNA seq analysis, ** p<0.01, *** p<0.001. (72C) Results of KEGG pathway analysis of bulk RNA-seq data from PBMCs cultured for 96 hours in the presence of V4-C26 hIgG4 or IgG4 isotype control, showing enrichment of transcript levels in genes associated with the TLR, TNFα, JAK-STAT and IL-17 signalling pathways. (72D) Enrichment of transcript levels in genes associated with type-I and Type-II interferon genes from bulk RNA-sequencing of MLR samples, for reactions performed in the presence of V4-C26 hIgG4 (HMBD-002), IgG4 isotype control or pembrolizumab (n=10; data represented as mean+SD and the p-adjusted values were obtained from bulk RNA seq analysis as *p<0.05, *** p<0.001).

FIGS. 73A to 73F. Graphs and bar charts showing V4-C26 hIgG4 anti-tumour responses in cell-derived xenograft (CDX) models. Mice were randomized and dosed with concentrations of test articles at indicated time-points. Tumour volumes were measured twice a week. Each data point represents the mean tumour volume +/−SEM from n=10 mice. (73A) Results in a syngeneic CDX model in which female BALB/c mice were subcutaneously implanted with CT26 cells. (73B) Results in a syngeneic CDX model in which female BALB/c mice were orthotopically implanted with VISTA overexpressing 4T-1 cells. (73C) Results in a CDX model in which CD34-engrafted humanized female HiMice were subcutaneously implanted with HCT15 cells. (73D) Results in a CDX model in which CD34-engrafted humanized female HiMice were subcutaneously implanted with A549 cells. (73E) Results of cellular profiling by flow cytometry, showing tumour-infiltrating leukocyte (TIL) populations in the tumor microenvironment in the CT26 CDX model (data shown are mean of n=3 and error bars are SEM). (73F) Results of a CT26 antigen recall assay measured by ex vivo culture of TILs (CD45+) with CT26 cells to measure lysis, or culture of tumour-enriched T cells (CD4+/CD8+) with CT26 cells to measure T cell activation via IFN-γ secretion using ELISA (data shown are mean of n=3 and error bars are SEM. All p-values were obtained by unpaired t-tests, *p<0.05, ** p<0.01; each data point represents a mouse).

FIGS. 74A to 74G. Graphs relating to pharmacokinetics and toxicity of V4-C26 hIgG4. (74A to 74C) Serum concentration of V4-C26 hIgG4 in tumor-bearing and non-tumor bearing (74A) Balb/c mice, (74B) Sprague Dawley rats, and (74C) Cynomolgus monkeys. V4-C26 hIgG4 was administered in a single dose at the indicated concentration, via intraperitoneal injection. (74D to 74G) Results from ex vivo cytokine release assays performed with (74D and 74E) human whole blood, or (74F and 74G) isolated PBMCs obtained from healthy donors (n=10), showing the level of (74D, 74F) IL-2 and (74E, 74G) IL-6 after stimulation for 24 hours with the indicated amount of V4-C26 hIgG4, IgG4 isotype control, (74D and 74E) staphylococcal enterotoxin B (SEB) or (74F and 74G) anti-CD3 antibody.

FIGS. 75A to 75E. Sensorgrams and calculated Kₒₙ, K_{off} and K_{D} for binding of V4-C26 hIgG4 to (75A) human VISTA, (75B) Non-human primate (NHP) VISTA, (75C) Rat VISTA, and (75D) Mouse VISTA. (75E) Graph and table showing of V4-C26 hIgG4 to human VISTA across pH range 5.5-7.5, as determined by ELISA (data shown are mean of n=3 measurements and error bars are SEM).

FIGS. 76A to 76H. Graphs showing results from high temperature stability study for HMBD-002-V4C26 in different formulations F1 to F10. (76A) Protein content by A280 UV light absorbance, (76B) pH, (76C) Aggregation measured by HPLC-SEC, (76D) Representative results of HPLC-SEC for formulation F1, (76E) Charge Variance measured by HPLC-CEX, (76F) Representative results of HPLC-CEX for formulation F1, (76G) Antigen binding measured by ELISA, (76H) Osmolality.

FIG. 77. Graph showing results from high concentration stability study for HMBD-002-V4C26 in different formulations F1 to F10. Aggregation measured by HPLC-SEC.

FIG. 78. Graph showing aggregation in HMBD-002 formulations after Freeze-Thaw Stress Study, measured by HPLC-SEC.

FIG. 79. Graph showing correlation of pH and Total Charge Variance Change for formulations F1, F2, and F5.

FIG. 80. Graphs showing in vivo efficacy of V4-C26 hIgG4 monotherapy and combination therapy with anti-mPD-1 (RMP1-14) antibody in murine syngeneic tumour models CT26 and B16/BL6. Mice were treated with 500 μg (˜25 mg/kg) of HMBD-002 alone or in combination with 200 μg (˜10 mg/kg) of RMP1-14.

FIG. 81. Graph showing in vivo efficacy of different doses of V4-C26 hIgG4 monotherapy in murine tumour model CT26.

FIGS. 82A to 82C. Graphs showing pharmacokinetic profiles of different doses of V4-C26 hIgG4 monotherapy in (82A) tumour-bearing and non-tumour-bearing mice, (82B) in male and female Sprague-Dawley rats, (82C) male and female cynomolgus monkeys.

FIGS. 83A and 83B. Graphs showing the anti-tumour responses of HMBD-002 (V4-C26 hIgG4), anti-PD-1 antibody (aPD1), or combination treatment with HMBD-002 and anti-PD-1 antibody in cell-derived xenograft (CDX) models. (83A) Results in a CDX model in which CD34-engrafted humanized HiMice were subcutaneously implanted with A549 cells. (83B) Results in a CDX model in which female BALB/c mice were subcutaneously implanted with CT26 cells.

### EXAMPLES

In the following Examples, the inventors describe the generation of novel anti-VISTA antibody clones targeted to specific regions of interest in the VISTA molecule, and the biophysical and functional characterisation and therapeutic evaluation of these antigen-binding molecules.

### Example 1: VISTA Target Design and Anti-VISTA Antibody Hybridoma Production

The inventors selected regions in the extracellular region of human VISTA (SEQ ID NO:3) for raising VISTA-binding monoclonal antibodies.

The FG loop region was targeted because this region of VISTA has been proposed to be important for VISTA's inhibitory function (Vigdorovich et al., Structure. 2013; 21(5): 707-717). The front-facing β-sheet region of VISTA was also targeted.

### 1.1 Hybridoma Production

Approximately 6 week old female BALB/c mice were obtained from InVivos (Singapore). Animals were housed under specific pathogen-free conditions and were treated in compliance with the Institutional Animal Care and Use Committee (IACUC) guidelines.

For hybridoma production, mice were immunized with proprietary mixtures of antigenic peptide, recombinant target protein or cells expressing the target protein.

Prior to harvesting the spleen for fusion, mice were boosted with antigen mixture for three consecutive days. 24 h after the final boost total splenocytes were isolated and fused with the myeloma cell line P3X63.Ag8.653 (ATCC, USA), with PEG using ClonaCell-HY Hybridoma Cloning Kit, in accordance with the manufacturer's instructions (Stemcell Technologies, Canada).

Fused cells were cultured in ClonaCell-HY Medium C (Stemcell Technologies, Canada) overnight at 37° C. in a 5% CO₂ incubator. The next day, fused cells were centrifuged and resuspended in 10 ml of ClonaCell-HY Medium C and then gently mixed with 90 ml of semisolid methylcellulose-based ClonaCell-HY Medium D (StemCell Technologies, Canada) containing HAT components, which combines the hybridoma selection and cloning into one step.

The fused cells were then plated into 96 well plates and allowed to grow at 37° C. in a 5% CO₂ incubator. After 7-10 days, single hybridoma clones were isolated and antibody producing hybridomas were selected by screening the supernatants by Enzyme-linked immunosorbent assay (ELISA) and Fluorescence-activated cell sorting (FACs).

### 1.2 Antibody Variable Region Amplification and Sequencing

Total RNA was extracted from hybridoma cells using TRIzol reagent (Life Technologies, Inc., USA) using manufacturer's protocol. Double-stranded cDNA was synthesized using SMARTer RACE 5′/3′ Kit (Clontech™, USA) in accordance with the manufacturer's instructions. Briefly, 1 μg total RNA was used to generate full-length cDNA using 5′-RACE CDS primer (provided in the kit), and the 5′ adaptor (SMARTer II A primer) was then incorporated into each cDNA according to manufacturer's instructions. cDNA synthesis reactions contained: 5× First-Strand Buffer, DTT (20 mM), dNTP Mix (10 mM), RNase Inhibitor (40 U/μl) and SMARTScribe Reverse Transcriptase (100 U/μl).

The race-ready cDNAs were amplified using SeqAmp DNA Polymerase (Clontech™, USA). Amplification reactions contained SeqAmp DNA Polymerase, 2× Seq AMP buffer, 5′ universal primer provided in the 5′ SMARTer Race kit, that is complement to the adaptor sequence, and 3′ primers that anneal to respective heavy chain or light chain constant region primer. The 5′ constant region were designed based on previously reported primer mix either by Krebber et al. J. Immunol. Methods 1997; 201:35-55, Wang et al. Journal of Immunological Methods 2000, 233; 167-177 or Tiller et al. Journal of Immunological Methods 2009; 350:183-193. The following thermal protocol was used: pre-denature cycle at 94° C. for 1 min; 35 cycles of 94° C., 30 s, 55° C., 30 s and 72° C., 45 s; final extension at 72° C. for 3 min.

The resulting VH and VL PCR products, approximately 550 bp, were cloned into pJET1.2/blunt vector using CloneJET PCR Cloning Kit (Thermo Scientific, USA) and used to transform highly competent E. coli DH5α. From the resulting transformants, plasmid DNA was prepared using Miniprep Kit (Qiagene, Germany) and sequenced. DNA sequencing was carried out by AITbiotech. These sequencing data were analyzed using the international IMGT (ImMunoGeneTics) information system (LeFranc et al., Nucleic Acids Res. (2015) 43 (Database issue): D413-22) to characterize the individual CDRs and framework sequences. The signal peptide at 5′ end of the VH and VL was identified by SignalP (v 4.1; Nielsen, in Kihara, D (ed): Protein Function Prediction (Methods in Molecular Biology vol. 1611) 59-73, Springer 2017).

Monoclonal anti-VISTA antibody clones were then selected for further development and characterisation. Humanised versions of antibody clone 4M2-C12 (also referred to herein as “V4”) were also prepared according to standard methods by cloning the CDRs of antibodies into VH and VL comprising human antibody framework regions.

| | | Peptide immunogen used |
|---|---|---|
| Antibody clone | VH/VL sequence | to raise the antibody |
| 4M2-C12 (also | VH = SEQ ID NO: 32 | SEQ ID NO: 26 |
| referred to herein | VL = SEQ ID NO: 40 | |
| as “V4”) | | |
| V4H1 | VH = SEQ ID NO: 52 | |
| | VL = SEQ ID NO: 57 | |
| V4H2 | VH = SEQ ID NO: 62 | |
| | VL = SEQ ID NO: 66 | |
| 4M2-B4 | VH = SEQ ID NO: 48 | |
| | VL = SEQ ID NO: 50 | |
| 4M2-C9 | VH = SEQ ID NO: 87 | |
| | VL = SEQ ID NO: 95 | |
| 4M2-D9 | VH = SEQ ID NO: 106 | |
| | VL = SEQ ID NO: 113 | |
| 4M2-D5 | VH = SEQ ID NO: 143 | |
| | VL = SEQ ID NO: 150 | |
| 4M2-A8 | VH = SEQ ID NO: 157 | |
| | VL = SEQ ID NO: 164 | |
| 2M1-B12 | VH = SEQ ID NO: 71 | SEQ ID NO: 27 |
| | VL = SEQ ID NO: 79 | |
| 2M1-D2 | VH = SEQ ID NO: 102 | |
| | VL = SEQ ID NO: 104 | |
| 1M2-D2 | VH = SEQ ID NO: 119 | SEQ ID NO: 28 |
| | VL = SEQ ID NO: 126 | |
| 13D5p | VH = SEQ ID NO: 183 | |
| | VL = SEQ ID NO: 188 | |
| 13D5-1 | VH = SEQ ID NO: 194 | |
| | VL = SEQ ID NO: 196 | |
| 13D5-13 | VH = SEQ ID NO: 199 | |
| | VL = SEQ ID NO: 202 | |
| 5M1-A11 | VH = SEQ ID NO: 133 | SEQ ID NO: 29 |
| | VL = SEQ ID NO: 136 | |
| 9M2-C12 | VH = SEQ ID NO: 168 | SEQ ID NO: 30 |
| | VL = SEQ ID NO: 176 | |

### Example 2: Antibody Production and Purification

2.1 Cloning VH and VL into Expression Vectors:

DNA sequences encoding the heavy and light chain variable regions of the anti-VISTA antibody clones were subcloned into the pmAbDZ_IgG1_CH and pmAbDZ_IgG1_CL (InvivoGen, USA) eukaryotic expression vectors for construction of human-mouse chimeric antibodies.

Alternatively, DNA sequence encoding the heavy and light chain variable regions of the anti-VISTA antibody clones were subcloned into the pFUSE-CHIg-hG1 and pFUSE2ss-CLIg-hk (InvivoGen, USA) eukaryotic expression vectors for construction of human-mouse chimeric antibodies. Human IgG1 constant region encoded by pFUSE-CHIg-hG1 comprises the substitutions D356E, L358M (positions numbered according to EU numbering) in the CH3 region relative to Human IgG1 constant region (IGHG1; UniProt: P01857-1, v1; SEQ ID NO:210). pFUSE2ss-CLIg-hk encodes human IgG1 light chain kappa constant region (IGCK; UniProt: P01834-1, v2).

Variable regions along with the signal peptides were amplified from the cloning vector using SeqAmp enzyme (Clontech™, USA) following the manufacturer's protocol. Forward and reverse primers having 15-20 bp overlap with the appropriate regions within VH or VL plus 6 bp at 5′ end as restriction sites were used. The DNA insert and the pFuse vector were digested with restriction enzyme recommended by the manufacturer to ensure no frameshift was introduced (e.g., EcoRI and NheI for VH, AgeI and BsiWI for VL) and ligated into its respective plasmid using T4 ligase enzyme (Thermo Scientific, USA). The molar ratio of 3:1 of DNA insert to vector was used for ligation.

### 2.2 Expression of Antibodies in Mammalian Cells

Antibodies were expressed using either 1) Expi293 Transient Expression System Kit (Life Technologies, USA), or 2) HEK293-6E Transient Expression System (CNRC-NRC, Canada) following the manufacturer's instructions.

### 1) Expi293 Transient Expression System:

### Cell Line Maintenance:

HEK293F cells (Expi293F) were obtained from Life Technologies, Inc (USA). Cells were cultured in serum-free, protein-free, chemically defined medium (Expi293 Expression Medium, Thermo Fisher, USA), supplemented with 50 IU/ml penicillin and 50 μg/ml streptomycine (Gibco, USA) at 37° C., in 8% CO₂ and 80% humidified incubators with shaking platform.

### Transfection:

Expi293F cells were transfected with expression plasmids using ExpiFectamine 293 Reagent kit (Gibco, USA) according to its manufacturer's protocol. Briefly, cells at maintenance were subjected to a media exchange to remove antibiotics by spinning down the culture, cell pellets were re-suspended in fresh media without antibiotics at 1 day before transfection. On the day of transfection, 2.5×10⁶/ml of viable cells were seeded in shaker flasks for each transfection. DNA-ExpiFectamine complexes were formed in serum-reduced medium, Opti-MEM (Gibco, USA), for 25 min at room temperature before being added to the cells. Enhancers were added to the transfected cells at 16-18 h post transfection. An equal amount of media was topped up to the transfectants at day 4 post-transfection to prevent cell aggregation. Transfectants were harvested at day 7 by centrifugation at 4000×g for 15 min, and filtered through 0.22 μm sterile filter units.

### 2) HEK293-6E Transient Expression System

### Cell Line Maintenance:

HEK293-6E cells were obtained from National Research Council Canada. Cells were cultured in serum-free, protein-free, chemically defined Freestyle F17 Medium (Invitrogen, USA), supplemented with 0.1% Kolliphor-P188 and 4 mM L-Glutamine (Gibco, USA) and 25 μg/ml G-418 at 37° C., in 5% CO₂ and 80% humidified incubators with shaking platform.

### Transfection:

HEK293-6E cells were transfected with expression plasmids using PEIpro™ (Polyplus, USA) according to its manufacturer's protocol. Briefly, cells at maintenance were subjected to a media exchange to remove antibiotics by centrifugation, cell pellets were re-suspended with fresh media without antibiotics at 1 day before transfection. On the day of transfection, 1.5-2×10⁶ cells/ml of viable cells were seeded in shaker flasks for each transfection. DNA and PEIpro™ were mixed to a ratio of 1:1 and the complexes were allowed to form in F17 medium for 5 min at RT before adding to the cells. 0.5% (w/v) of Tryptone N1 was fed to transfectants at 24-48 h post transfection. Transfectants were harvested at day 6-7 by centrifugation at 4000×g for 15 min and the supernatant was filtered through 0.22 μm sterile filter units.

Cells were transfected with vectors encoding the following combinations of polypeptides:

| Antigen- | | |
|---|---|---|
| biding | | |
| molecule | Polypeptides | Antibody |
| [1] | 4M2-C12 VH-CH1-CH2-CH3 (SEQ ID NO: 212) | anti-VISTA clone 4M2-C12 IgG1 |
| | + | |
| | 4M2-C12 VL-CK (SEQ ID NO: 213) | |
| [2] | 4M2-B4 VH-CH1-CH2-CH3 (SEQ ID NO: 214) | anti-VISTA clone 4M2-B4 IgG1 |
| | + | |
| | 4M2-B4 VL-CK (SEQ ID NO: 215) | |
| [3] | V4H1 VH-CH1-CH2-CH3 (SEQ ID NO: 216) | anti-VISTA clone V4H1 IgG1 |
| | + | |
| | V4H1 VL-CK (SEQ ID NO: 217) | |
| [4] | V4H2 VH-CH1-CH2-CH3 (SEQ ID NO: 218) | anti-VISTA clone V4H2 IgG1 |
| | + | |
| | V4H2 VL-CK (SEQ ID NO: 219) | |
| [5] | 2M1-B12 VH-CH1-CH2-CH3 (SEQ ID NO: 220) | anti-VISTA clone 2M1-B12 IgG1 |
| | + | |
| | 2M1-B12 VL-CK (SEQ ID NO: 221) | |
| [6] | 4M2-C9 VH-CH1-CH2-CH3 (SEQ ID NO: 222) | anti-VISTA clone 4M2-C9 IgG1 |
| | + | |
| | 4M2-C9 VL-CK (SEQ ID NO: 223) | |
| [7] | 2M1-D2 VH-CH1-CH2-CH3 (SEQ ID NO: 224) | anti-VISTA clone 2M1-D2 IgG1 |
| | + | |
| | 2M1-D2 VL-CK (SEQ ID NO: 225) | |
| [8] | 4M2-D9 VH-CH1-CH2-CH3 (SEQ ID NO: 226) | anti-VISTA clone 4M2-D9 IgG1 |
| | + | |
| | 4M2-D9 VL-CK (SEQ ID NO: 227) | |
| [9] | 1M2-D2 VH-CH1-CH2-CH3 (SEQ ID NO: 228) | anti-VISTA clone 1M2-D2 IgG1 |
| | + | |
| | 1M2-D2 VL-CK (SEQ ID NO: 229) | |
| [10] | 5M1-A11 VH-CH1-CH2-CH3 (SEQ ID NO: 230) | anti-VISTA clone 5M1-A11 IgG1 |
| | + | |
| | 5M1-A11 VL-CK (SEQ ID NO: 231) | |
| [11] | 4M2-D5 VH-CH1-CH2-CH3 (SEQ ID NO: 232) | anti-VISTA clone 4M2-D5 IgG1 |
| | + | |
| | 4M2-D5 VL-CK (SEQ ID NO: 233) | |
| [12] | 4M2-A8 VH-CH1-CH2-CH3 (SEQ ID NO: 234) | anti-VISTA clone 4M2-A8 IgG1 |
| | + | |
| | 4M2-A8 VL-CK (SEQ ID NO: 235) | |
| [13] | 9M2-C12 VH-CH1-CH2-CH3 (SEQ ID NO: 236) | anti-VISTA clone 9M2-C12 IgG1 |
| | + | |
| | 9M2-C12 VL-CK (SEQ ID NO: 237) | |
| [14] | 13D5p VH-CH1-CH2-CH3 (SEQ ID NO: 238) | anti-VISTA clone 13D5p IgG1 |
| | + | |
| | 13D5p VL-CK (SEQ ID NO: 239) | |
| [15] | 13D5-1 VH-CH1-CH2-CH3 (SEQ ID NO: 240) | anti-VISTA clone 13D5-1 IgG1 |
| | + | |
| | 13D5-1 VL-CK (SEQ ID NO: 241) | |
| [16] | 13D5-13 VH-CH1-CH2-CH3 (SEQ ID NO: 242) | anti-VISTA clone 13D5-13 IgG1 |
| | + | |
| | 13D5-13 VL-CK (SEQ ID NO: 243) | |

### 2.3 Antibody Purification

### Affinity Purification, Buffer Exchange and Storage:

Antibodies secreted by the transfected cells into the culture supernatant were purified using liquid chromatography system AKTA Start (GE Healthcare, UK). Specifically, supernatants were loaded onto HiTrap Protein G column (GE Healthcare, UK) at a binding rate of 5 ml/min, followed by washing the column with 10 column volumes of washing buffer (20 mM sodium phosphate, pH 7.0). Bound mAbs were eluted with elution buffer (0.1 M glycine, pH 2.7) and the eluents were fractionated to collection tubes which contain appropriate amount of neutralization buffer (1 M Tris, pH 9). Neutralised elution buffer containing purified mAb were exchanged into PBS using 30K MWCO protein concentrators (Thermo Fisher, USA) or 3.5K MWCO dialysis cassettes (Thermo Fisher, USA). Monoclonal antibodies were sterilized by passing through 0.22 μm filter, aliquoted and snap-frozen in −80° C. for storage.

### 2.4 Antibody-Purity Analysis

### Size Exclusion Chromatography (SEC):

Antibody purity was analysed by size exclusion chromatography (SEC) using Superdex 200 10/30 GL columns (GE Healthcare, UK) in PBS running buffer, on a AKTA Explorer liquid chromatography system (GE Healthcare, UK). 150 μg of antibody in 500 μl PBS pH 7.2 was injected to the column at a flow rate of 0.75 ml/min at room temperature. Proteins were eluted according to their molecular weights.

The result for anti-VISTA antibody clone V4 ([1] of Example 2.2) is shown in FIG. 10.

### Sodium-Dodecyl Sulfate Polyacrylamide Gel Electrophoresis (SDS-PAGE):

Antibody purity was also analysed by SDS-PAGE under reducing and non-reducing conditions according to standard methods. Briefly, 4%-20% TGX protein gels (Bio-Rad, USA) were used to resolve proteins using a Mini-Protean Electrophoresis System (Bio-Rad, USA). For non-reducing condition, protein samples were denatured by mixing with 2× Laemmli sample buffer (Bio-Rad, USA) and boiled at 95° C. for 5-10 min before loading to the gel. For reducing conditions, 2× sample buffer containing 5% of β-mercaptoethanol (BME), or 40 mM DTT (dithiothreitol) was used. Electrophoresis was carried out at a constant voltage of 150V for 1 h in SDS running buffer (25 mM Tris, 192 mM glycine, 1% SDS, pH 8.3).

### Western Blot:

Protein samples (30 μg) were fractionated by SDS-PAGE as described above and transferred to nitrocellulose membranes. Membranes were then blocked and immunoblotted with antibodies overnight at 4° C. After washing three times in PBS-Tween the membranes were then incubated for 1 h at room temperature with horseradish peroxidase (HRP)-conjugated secondary antibodies. The results were visualized via a chemiluminescent Pierce ECL Substrate Western blot detection system (Thermo Scientific, USA) and exposure to autoradiography film (Kodak XAR film).

The primary antibody used for detection was goat anti-mouse IgG(H+L) Antibody (LI-COR, Cat. No. 926-32210).

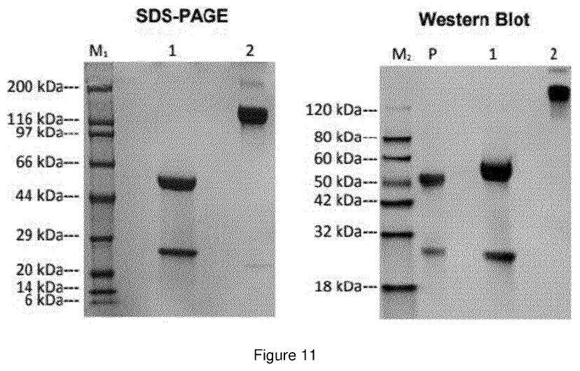

The result for anti-VISTA antibody clone V4 ([1] of Example 2.2) is shown in FIG. 11. V4 was easily expressed, purified and processed at high concentrations.

### Example 3: Biophysical Characterisation

### 3.1 Analysis of Cell Surface Antigen-Binding by Flow Cytometry

Wildtype HEK293T cells (which do not express high levels of VISTA) and cells of HEK293T cells transfected with vector encoding human VISTA (i.e. HEK 293 HER O/E cells) were incubated with 20 μg/ml of anti-VISTA antibody or isotype control antibody at 4° C. for 1 hr. The anti-VISTA antibody clone VSTB112, as described in WO 2015/097536, was included in the analysis as a positive control.

The cells were washed thrice with FACS buffer (PBS with 5 mM EDTA and 0.5% BSA) and resuspended in FITC-conjugated anti-FC antibody (Invitrogen, USA) for 40 min at 2-8° C. Cells were washed again and resuspended in 200 μL of FACS flow buffer (PBS with 5 mM EDTA) for flow cytometric analysis using MACSQuant 10 (Miltenyi Biotec, Germany). After acquisition, all raw data were analyzed using Flowlogic software. Cells were gated using forward and side scatter profile and Median of Fluorescence Intensity (MFI) value was determined for native and overexpressing cell populations.

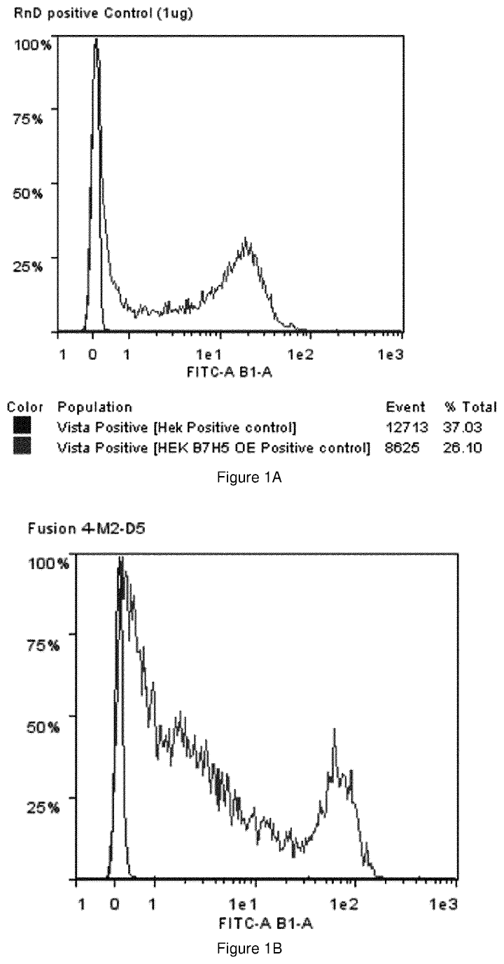

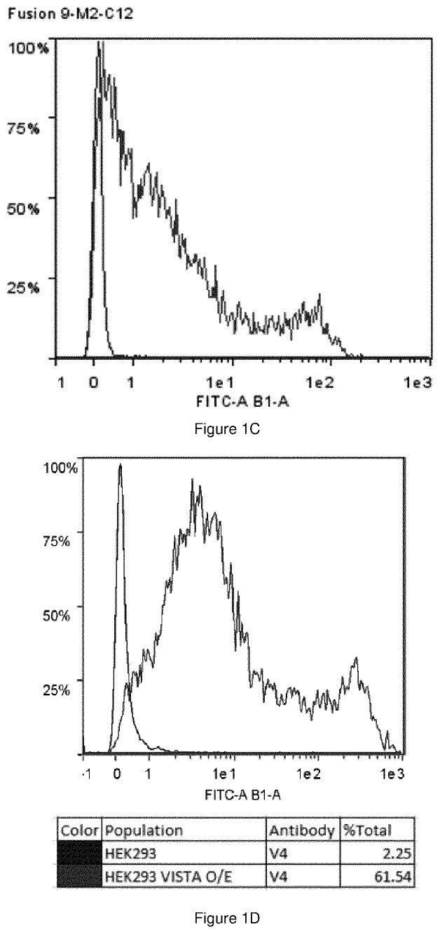

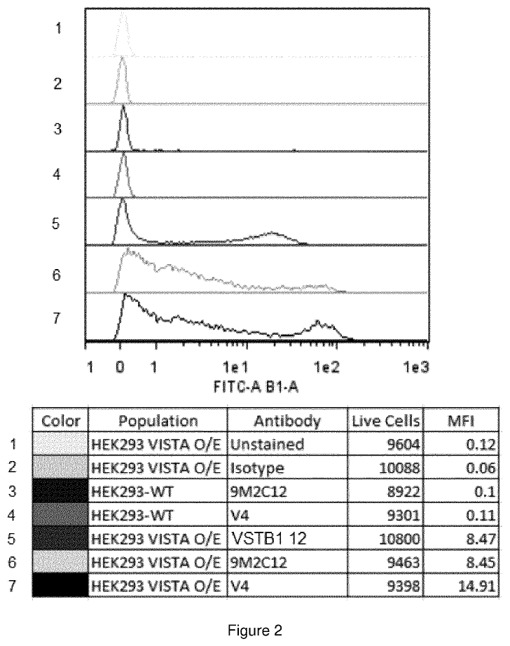

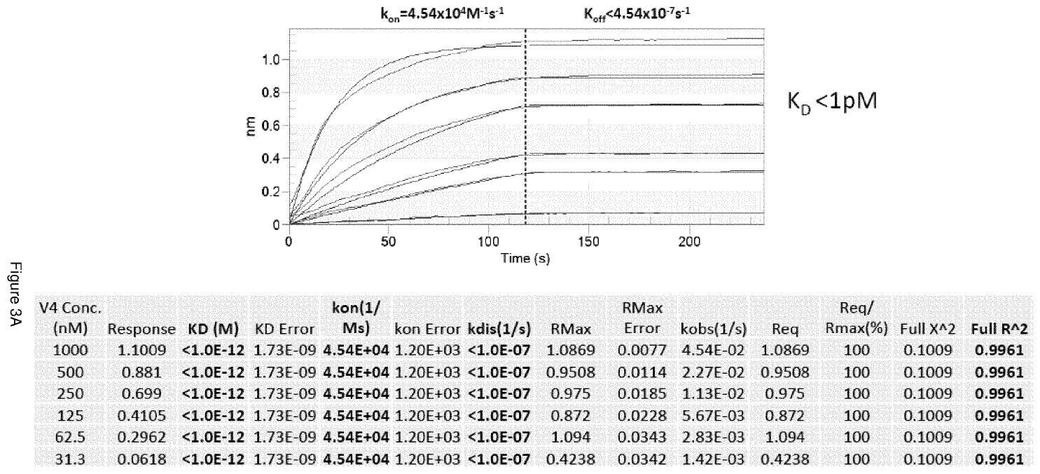

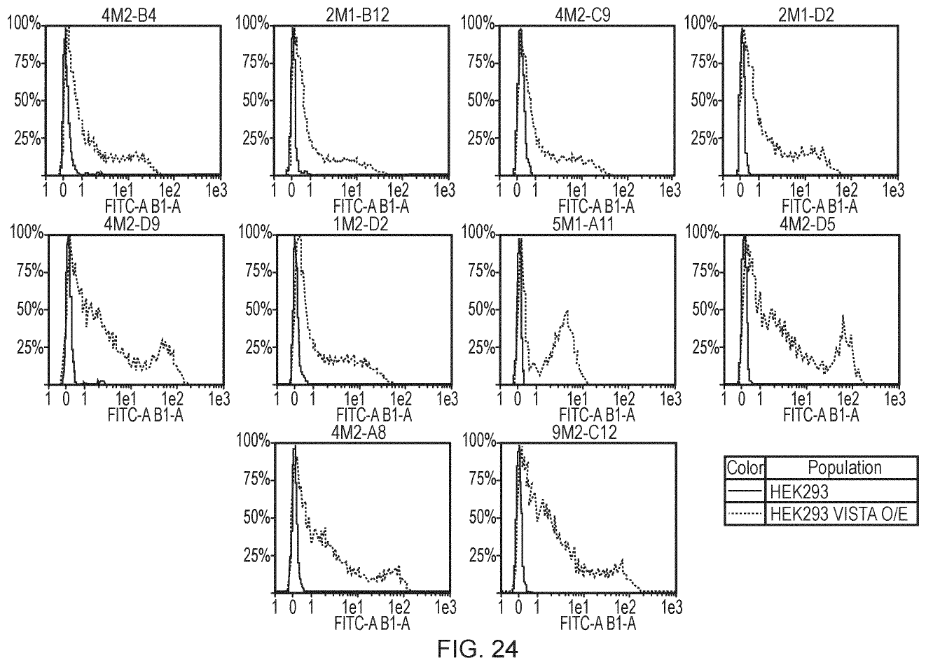

The results are shown in FIGS. 1A to 1D, FIG. 2 and FIG. 24. The anti-VISTA antibodies were shown to bind to human VISTA with high specificity.

In a separate experiment 13D5p ([14] of Example 2.2) was analysed for its ability to bind to cells transfected with vector encoding cynomolgus macaque VISTA or murine VISTA. 13D5p was found to display cross-reactivity with cynomolgus macaque VISTA and murine VISTA.

### 3.2 Global Affinity Study Using Octet QK384 System

Bio-Layer Interferometry (BLI) experiments were performed using the Octet QK384 system (ForteBio). anti-Penta-HIS (HIS1K) coated biosensor tips (Pall ForteBio, USA) were used to capture His-tagged human, cynomolgus macaque or murine VISTA (270 nM). All measurements were performed at 25° C. with agitation at 1000 rpm. Kinetic measurements for antigen binding were performed by loading anti-VISTA antibody at different concentrations (indicated in the Figures) for 120 s, followed by a 120 s dissociation time by transferring the biosensors into assay buffer containing wells. Sensograms were referenced for buffer effects and then fitted using the Octet QK384 user software (Pall ForteBio, USA).

Kinetic responses were subjected to a global fitting using a one site binding model to obtain values for association (kon), dissociation (koff) rate constants and the equilibrium dissociation constant (KD). Only curves that could be reliably fitted with the software (R²>0.90) were included in the analysis.

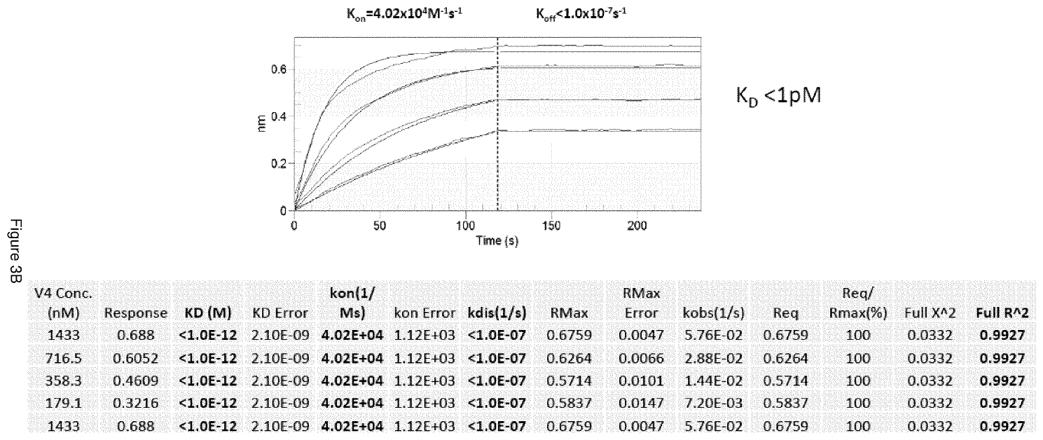

Representative sensorgrams for analysis of binding by anti-VISTA antibody clone V4 (i.e. [1] of example 2.2) are shown in FIGS. 3A to 3C.

Anti-VISTA antibody clone V4 was found to bind to human and cynomolgus macaque VISTA with an affinity of K_{D}=<1 pM, and to bind to murine VISTA with an affinity of K_{D}=113 nM.

### 3.3 ELISAs for Determining Antibody Specificity

ELISAs were used to determine the binding specificity of the antibodies. Anti-VISTA antibodies were analysed for binding to human VISTA polypeptide, respective mouse and cynomolgus macaque homologues, as well as human PD-L1 and human HER3 (Sino Biological Inc., China).

ELISAs were carried out according to standard protocols. Briefly, 96-well plates (Nunc, Denmark) were coated with 1 μg/ml of target protein in phosphate-buffered saline (PBS) for 2 h at 37° C. After blocking for 1 h with 10% BSA in Tris buffer saline (TBS) at room temperature, the test antibody was serially diluted (12 point serial dilution) with the highest concentration being 30 μg/ml and added to the plate, in the. Post 1 h incubation at room temperature, plates were washed three times with TBS containing 0.05% Tween 20 (TBS-T) and were then incubated with a HRP-conjugated anti-mouse IgG antibody (Life Technologies, Inc., USA) for 1 h at room temperature. After washing, plates were developed with colorimetric detection substrate 3,3′,5,5′-tetramethylbenzidine (Turbo-TMB; Pierce, USA) for 15 min at room temperature. The reaction was stopped with 2M H₂SO₄, and OD was measured at 450 nM within 30 min.

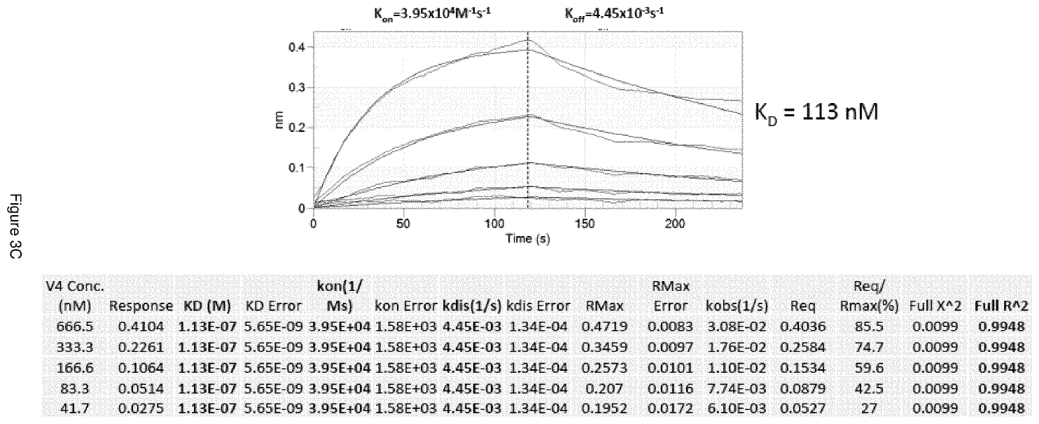

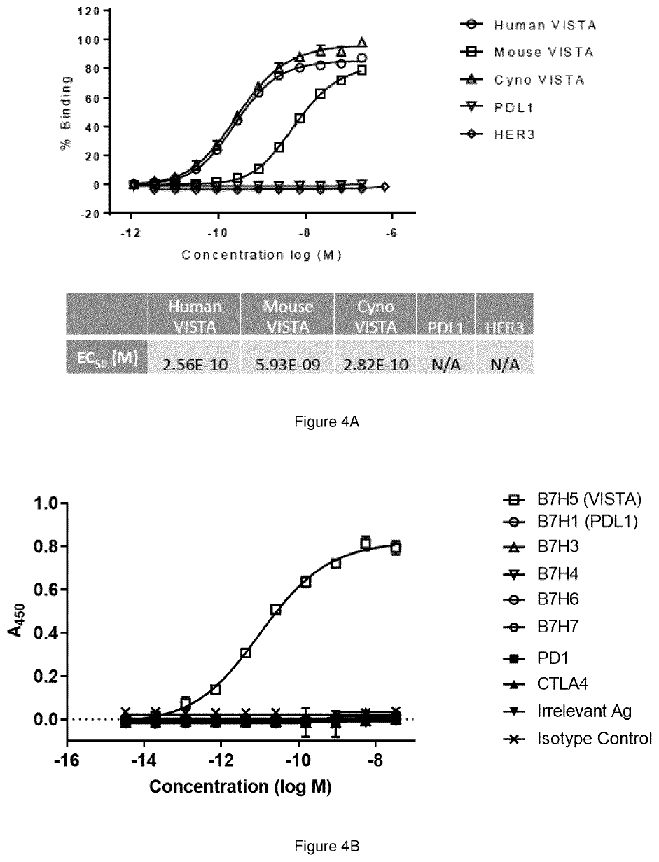

The results obtained with anti-VISTA antibody clone V4 ([1] of Example 2.2) are shown in FIG. 4A. Clone V4 was found to be able to bind to human, cynomolgus macaque and murine VISTA, but did not display cross-reactivity with human PD-L1 or human HER3 (even at very high concentrations).

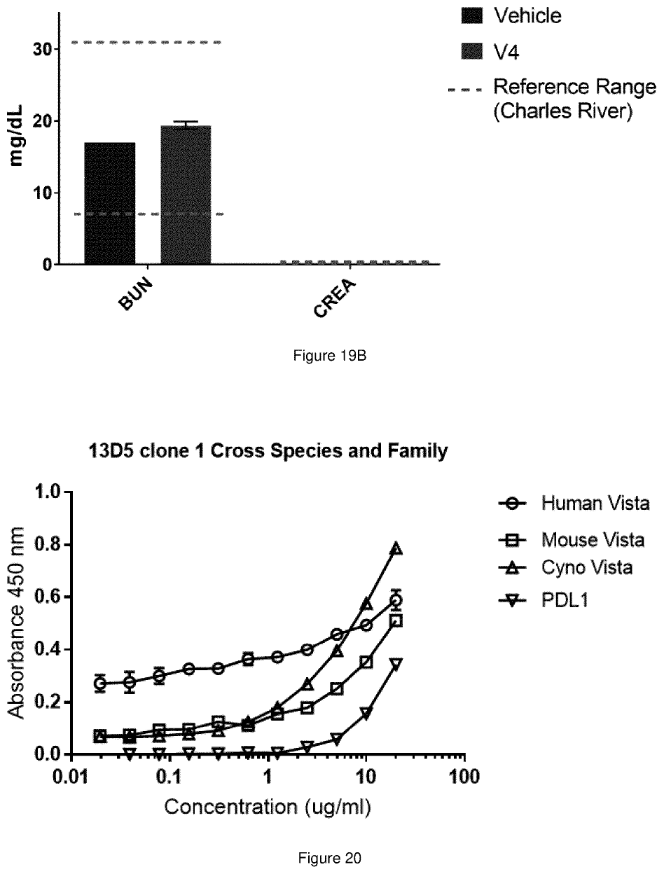

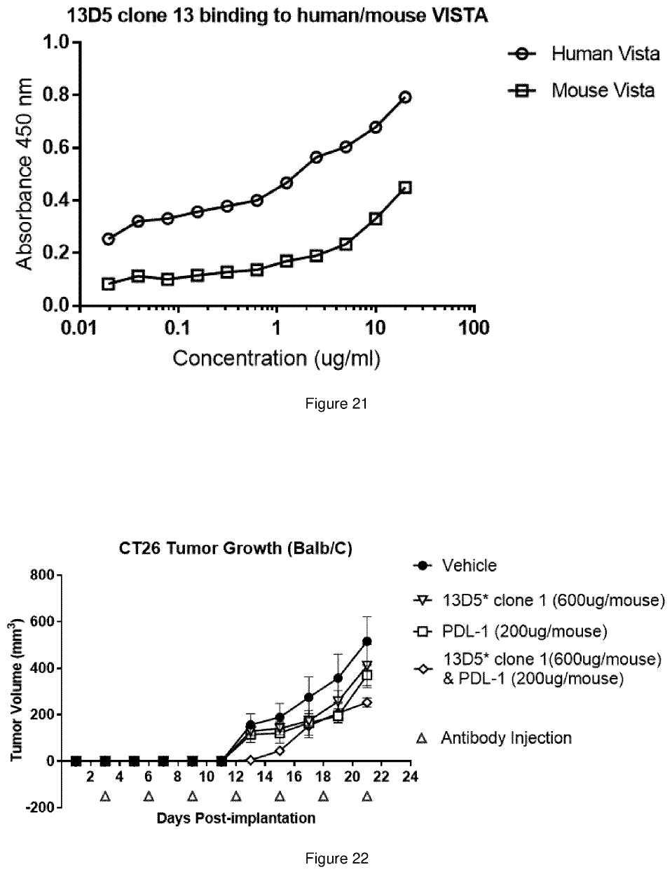

The results obtained with anti-VISTA antibody clone 13D5-1 ([15] of Example 2.2) are shown in FIG. 20. Clone 13D5-1 was found to be able to bind to human, cynomolgus macaque and mouse VISTA. The results obtained with anti-VISTA antibody clone 13D5-13 ([16] of Example 2.2) are shown in FIG. 21. Clone 13D5-13 was found to be able to bind to human and mouse VISTA.

In a further experiment, anti-VISTA antibody clone V4 ([1] of Example 2.2) was analysed by ELISA for ability to bind to human VISTA, PD-1, PD-L1, B7H3, B7H4, B7H6, B7H7 and CTLA4. The results are shown in FIG. 4C. Clone V4 was found not to cross-react with any of PD-1, PD-L1, B7H3, B7H4, B7H6, B7H7 or CTLA4.

### 3.4 Analysis of Thermostability by Differential Scanning Fluorimetry

Briefly, triplicate reaction mixes of antibodies at 0.2 mg/mL and SYPRO Orange dye (ThermoFisher) were prepared in 25 μL of PBS, transferred to wells of MicroAmp Optical 96-Well Reaction Plates (ThermoFisher), and sealed with MicroAmp Optical Adhesive Film (ThermoFisher). Melting curves were run in a 7500 fast Real-Time PCR system (Applied Biosystems) selecting TAMRA as reporter and ROX as passive reference. The thermal profile included an initial step of 2 min at 25° C. and a final step of 2 min at 99° C., with a ramp rate of 1.2%. The first derivative of the raw data was plotted as a function of temperature to obtain the derivative melting curves. Melting temperatures (Tm) of the antibodies were extracted from the peaks of the derivative curves.

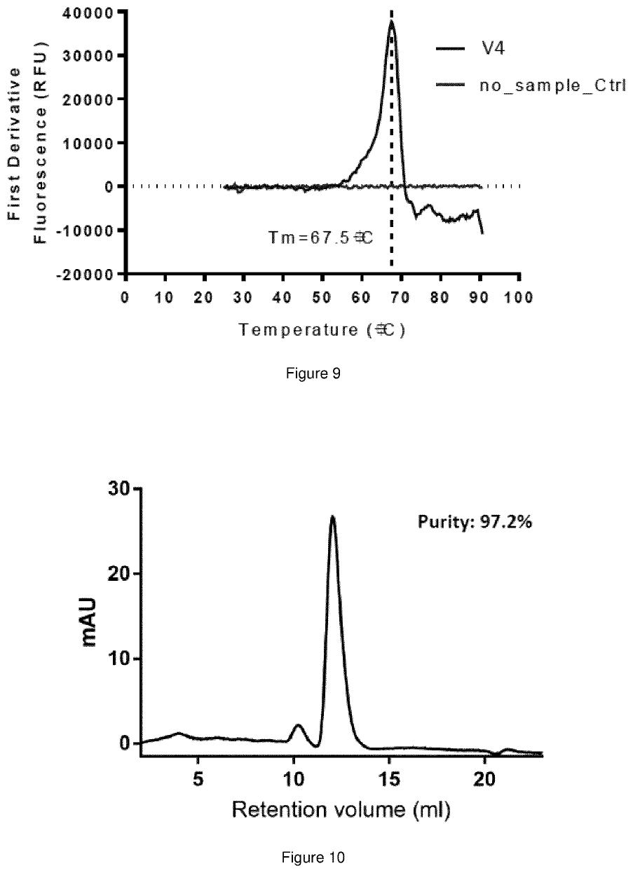

The first derivative of the raw data obtained for Differential Scanning Fluorimetry analysis of the thermostability of antibody clone V4 IgG1 format (i.e. [1] of Example 2.2) is shown in FIG. 9. The Tm was determined to be 67.5° C.

### Example 4: Functional Characterisation

4.1 Interaction between VISTA and VSIG-3

The inventors investigated whether VSIG-3 behaves as a ligand for VISTA by Bio-Layer Interferometry (BLI) analysis using the Octet QK384 system (ForteBio). Briefly, an anti-human Fc capture biosensor was used to capture Fc-tagged VSIG-3 at concentration 100 nM, and association of captured VSIG-3 with VISTA applied at concentrations starting from 3000 nM followed by 3 serial dilutions were measured, and compared to PBS control.

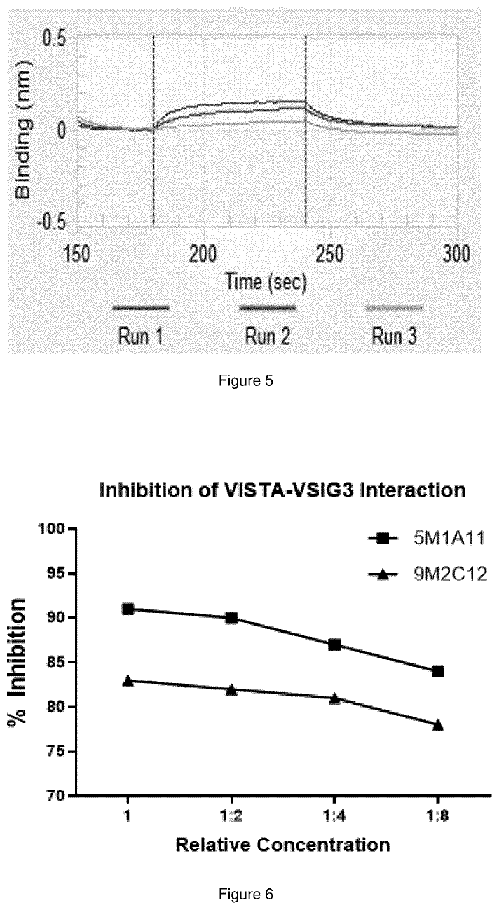

Representative sensorgrams are shown in FIG. 5. The affinity of association between VSIG-3 and VISTA was calculated to be ˜K_{D}=5.28 μM.

The inventors next analysed the ability of anti-VISTA antibodies to inhibit interaction between VISTA and VSIG-3.

Briefly, 96-well plates (Nunc, Denmark) were coated with 1 μg/ml of untagged or Fc-tagged VSIG-3 (R&D Systems, USA) in 1×PBS for 16 h at 4° C. After blocking for 1 h with 1% BSA in TBS at room temperature, 15 μg/ml of VISTA/human His-tagged fusion protein (Sinobiological Inc, China) was added in the presence or absence of increasing concentrations of anti-VISTA antibody, and incubated for 1 hr at room temperature. Plates were subsequently washed three times with TBS-T and incubated with an HRP-conjugated anti-his secondary antibody for 1 h at room temperature. After washing, plates were developed with colorimetric detection substrate Turbo-TMB (Pierce, USA). The reaction was stopped with 2M H₂SO₄, and OD was measured at 450 nM.

The results obtained for anti-VISTA antibody clones 5M1-A11 and 9M2-C12 ([10] and of Example 2.2) are shown in FIG. 6. The anti-VISTA antibodies displayed dose-dependent inhibition of interaction between VISTA and VSIG-3.

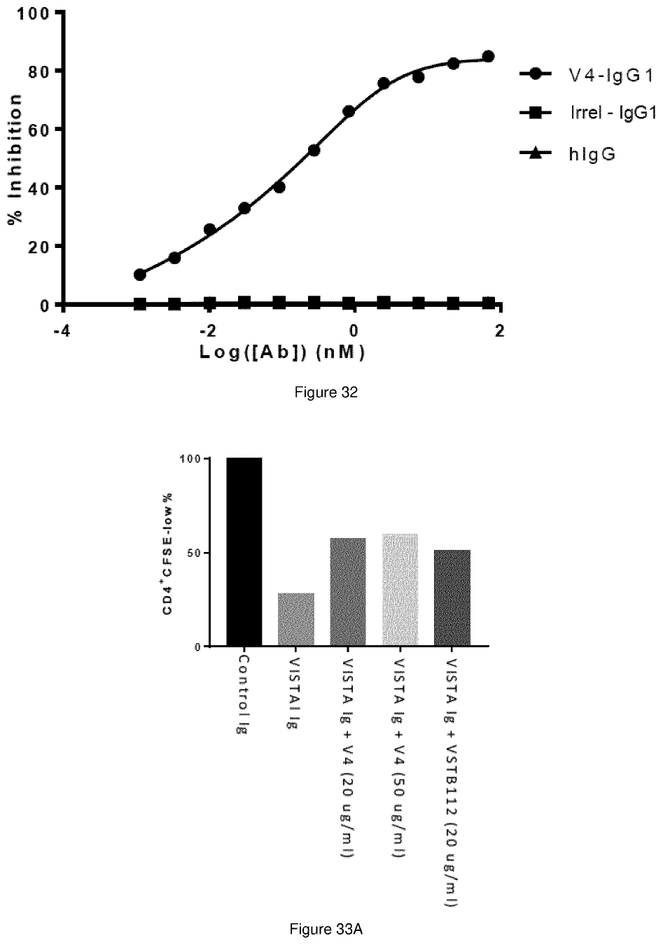

In a further experiment, inhibition by 4M2-C12 IgG1 ([1] of Example 2.2) of interaction between VISTA and VSIG-3 was analysed. Inhibition of VISTA: VSIG-3 interaction by an antibody specific for an irrelevant target antigen and by human IgG1 isotype control were also analysed as control conditions. The results are shown in FIG. 32. 4M2-C12 IgG1 was found to inhibit VISTA: VSIG-3 interaction in a dose-dependent manner.

In a further experiment, inhibition by 4M2-C12 IgG1 ([1] of Example 2.2) of interaction between VISTA and VSIG-3 was analysed in an assay in which VISTA-Fc was used as the capture agent. Briefly, wells of 384-well plates were coated with 30 μl of 0.5 μg/ml of VISTA-Fc for 1 h at room temperature. Plates were washed with PBS-T and blocked for 1 h with 1% BSA in TBS at room temperature. Serial dilutions of 4M2-C12 IgG1 or human IgG1 isotype control antibodies were added to plates, together with 0.3 μg/ml of VISG3-His. After 1 h of incubation at room temperature plates were washed five times with PBS-T, and incubated with goat anti-HIS-HRP for 1 h at room temperature. Plates were washed five times with PBS-T and, developed with Turbo-TMB. The reaction was stopped with 2M H₂SO₄, and OD was measured at 450 nM.

The results are shown in FIG. 54. 4M2-C12 IgG1 was found to inhibit VISTA: VSIG-3 interaction in a dose-dependent manner.

### 4.2 Interaction Between VISTA and PSGL-1

The inventors next investigated whether PSGL-1 behaves as a ligand for VISTA in a flow cytometry-based assay.

Briefly, 100,000 HEK293T cells modified to overexpress human VISTA protein (by transfection with a construct encoding human VISTA) were co-incubated with 4M2-C12-hIgG1 ([1] of Example 2.2) or an isotype-matched control antibody at concentrations of 20 μg/ml, 40 μg/ml or 80 μg/ml for 15 min at 4° C., in buffer comprising HBSS, 0.5% BSA and 2 mM EDTA pH 6.0. 15 μg/ml of Fc-tagged human PSGL1 (R&D Systems, Cat No: 3345-PS) or the same amount of an Fc-tagged irrelevant antigen was then added to the cells, which were then incubated for a further 45 min at 4° C. Cells were subsequently washed three times with buffer, and then FITC-conjugated anti-PSGL1 antibody (Miltenyi Biotec Cat No: 130-104-706) was added at a dilution factor of (1:11) or Alex488-conjugated anti-Fc antibody was added at a dilution factor of 1:200, and the cells were incubated for 15 min at 4° C. Cells were then washed three times with buffer and analysed by flow cytometry.

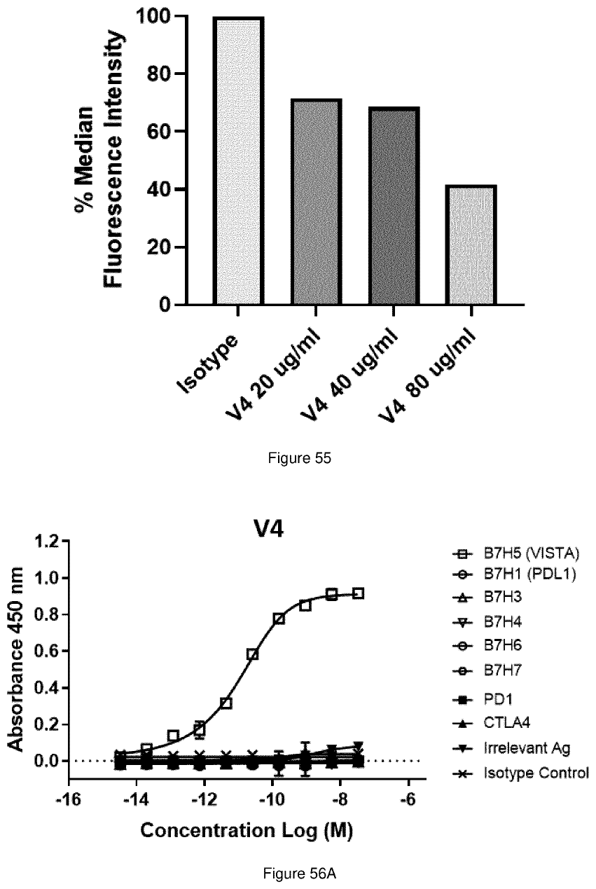

The results are shown in FIG. 55. 4M2-C12-hIgG1 was found to inhibit binding of PSGL-1 to VISTA in a dose-dependent manner.

### 4.3 Inhibition of VISTA-Mediated Signalling

The inventors investigated whether anti-VISTA antibody clone 13D5p could inhibit VISTA-mediated signalling by analysis using a mixed lymphocyte reaction (MLR) assay.

Briefly, PBMCs were isolated from unrelated donors (to obtain stimulator and effector populations) using Septamate kit (Stemcell Technologies, Canada), according to the manufacturer's instructions. Stimulator cells were treated with 50 μg/mL of mitomycin C (Sigma Aldrich, USA) for 20 minutes at 37° C. and used after 5 washes with 1×PBS. The stimulator population was seeded at 0.5×10⁵ cells/well and responder population at 1.0×10⁵ cells per well in the presence or absence of increasing concentrations of the test antibody, starting at a highest concentration of 20 μg/ml. After 5 days, the supernatant was harvested and the levels of IL-17, IL-2A and IFN-γ were determined by ELISA following the standard protocol.

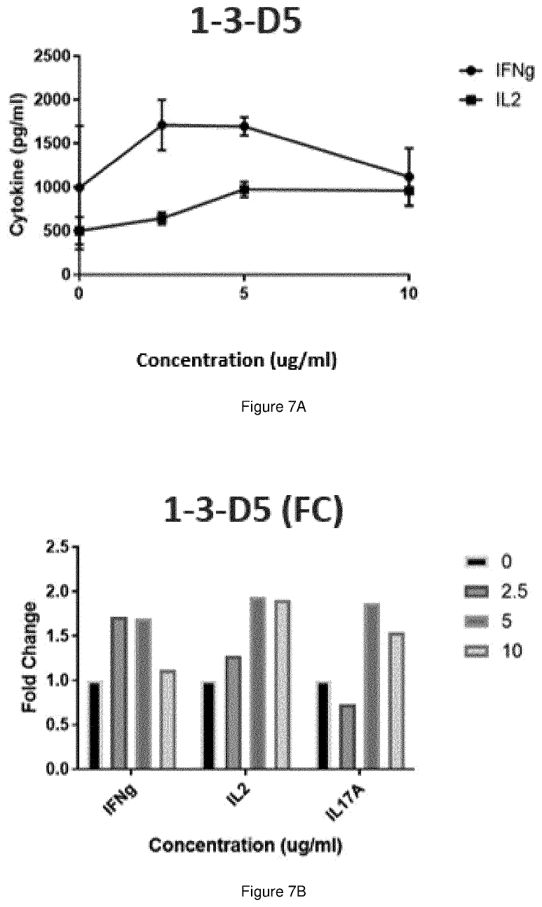

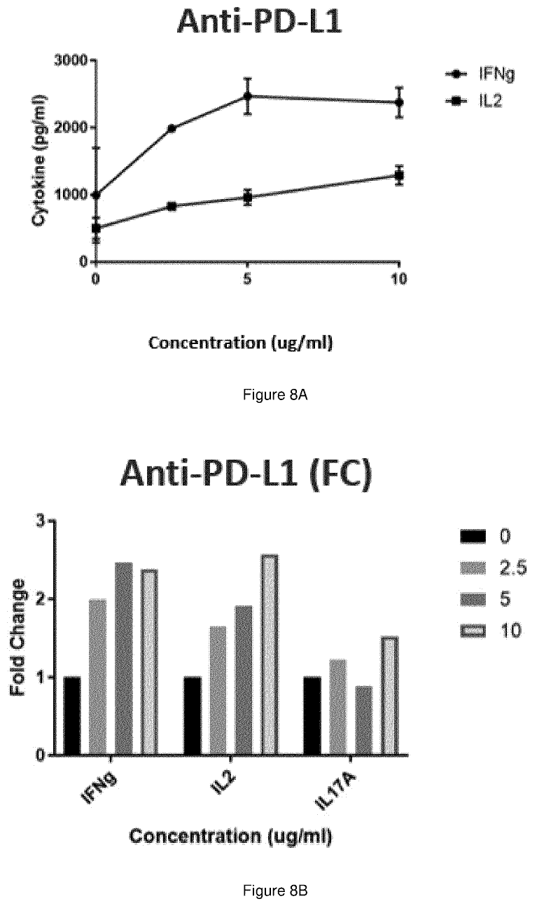

The results are shown in FIGS. 7A and 7B. Anti-VISTA antibody 13D5p was found to result in an increase in the levels of IL-17, IL-2 and IFN-γ. FIGS. 8A and 8B show results obtained in the same assay using anti-PD-L1 antibody clone MIH5 (ThermoFisher Scientific).

### Example 5: Analysis In Vivo

For in vivo studies, 4M2-C12 was produced in mouse IgG2a format. The molecule is a heteromer of the heavy chain polypeptide having the sequence shown in SEQ ID NO:248, and the light chain polypeptide having the sequence shown in SEQ ID NO:250. 4M2-C12 mIgG2a was produced by co-expression of nucleic acids encoding the heavy and light chains polypeptides in CHO cells, and was subsequently purified.

| Antigen- | | |
|---|---|---|
| biding | | |
| molecule | Polypeptides | Antibody |
| [17] | 4M2-C12 mIgG2a HC (SEQ ID NO: 248) | 4M2-C12 mIgG2a |
| | + | |
| | 4M2-C12 CL (SEQ ID NO: 250) | |

### 5.1 Pharmacokinetic Analysis

C57BL/6 mice approximately 6-8 weeks old were housed under specific pathogen-free conditions and were treated in compliance with the Institutional Animal Care and Use Committee (IACUC) guidelines.

600 μg anti-VISTA antibody was administered and blood was obtained from 3 mice by cardiac puncture at baseline (−2 hr), 0.5 hr, 6 hr, 24 hr, 96 hr, 168 hr and 336 hr after administration. Antibody in the serum was quantified be ELISA.

The parameters for the pharmacokinetic analysis were derived from a non-compartmental model: maximum concentration (Cₘₐₓ), AUC (0-336 hr), AUC (0-infinity), Half-life (t_{1/2}), Clearance (CL), Volume of distribution at steady state (Vₛₛ).

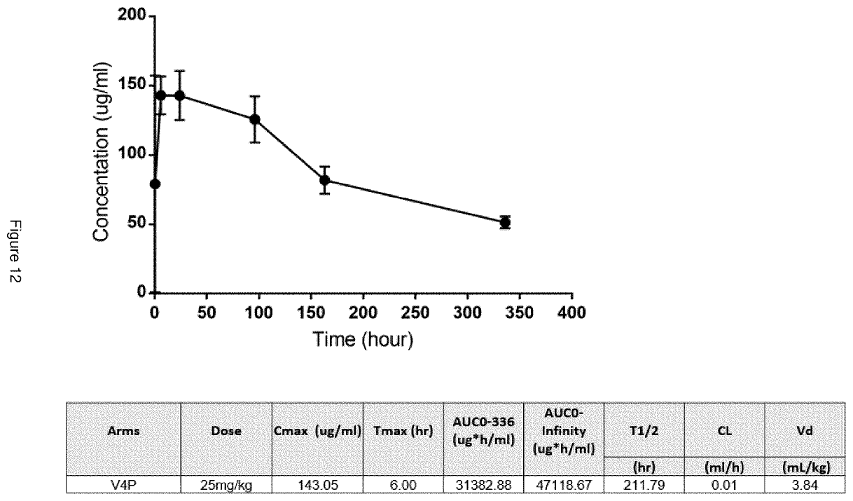

The results obtained for anti-VISTA antibody clone V4 ([17] of Example 5) are shown in FIG. 12. This antibody clone was found to have a half-life of 11.7 days.

### 5.2 Analysis of Efficacy to Treat Cancer In Vivo

Female BALB/c or C57BL/6 mice approximately 6-8 weeks old were purchased from InVivos (Singapore). Animals were housed under specific pathogen-free conditions and were treated in compliance with the Institutional Animal Care and Use Committee (IACUC) guidelines.

Cell lines used in the studies included LL2 cells (Lewis Lung carcinoma), 4T1 cells (breast cancer), CT26 cells (colon carcinoma), Clone-M3 cells (melanoma) and EL4 cells (T cell leukemia/lymphoma) obtained from ATCC. B16-BL6 cells (melanoma) were obtained from Creative Bioarray. The cell lines were maintained in accordance with the supplier's instructions; LL2 cells, B16-BL6 cells and EL4 cells were cultured in DMEM supplemented with 10% fetal bovine serum (FBS) and 1% Pen/Strep, and 4T1 cells and CT26 cells were cultured in RPMI-1640 supplemented with 10% FBS and 1 and 1% Pen/Strep. Clone-M3 cells were grown in F12-K medium supplemented with 2.5% FBS, 15% Horse serum and 1% Pen/Strep. All cells were cultured at 37° C. in a 5% CO₂ incubator.

Syngeneic tumor models were generated by injecting either LL2 (2×10⁵), 4T1 (5×10⁵), CT26 (1×10⁵-1×10⁶), Clone-M3 (5×10⁵), EL4 (2×10⁵) or B16-BL6 (1×10⁵) cells subcutaneously into the right flank of mice.

3 days post-implantation anti-VISTA antibodies were administered intraperitoneally every 3 days for a total of 6 doses. Control groups received vehicle treatment at the same dose interval.

Tumor volume was measured 3 times a week using a digital caliper and calculated using the formula [L×W2/2]. Study End point was considered to have been reached once the tumors of the control arm measured >1.5 cm in length.

### 5.2.1 CT26 Cell Model

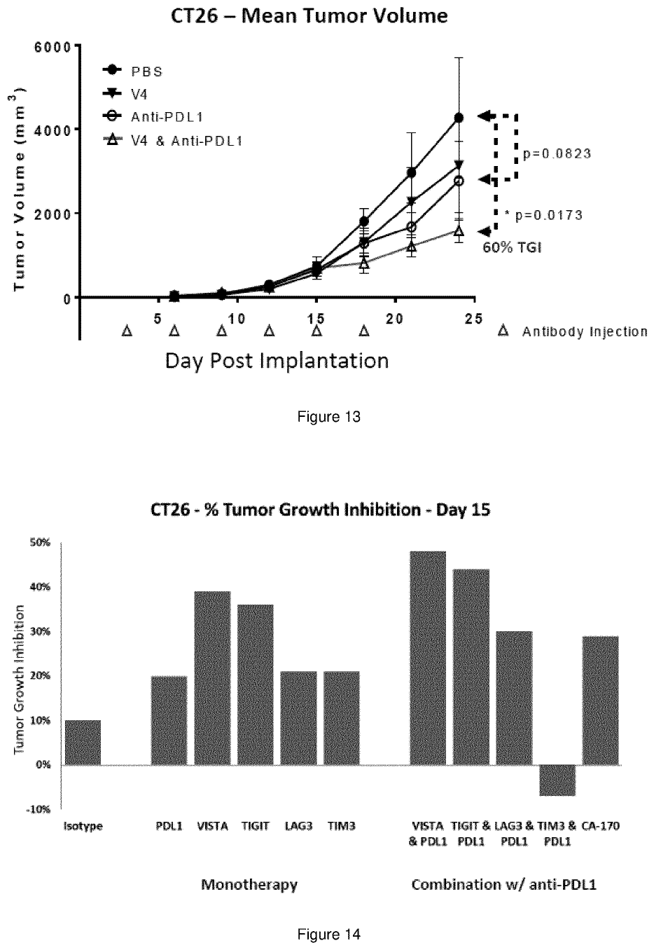

FIG. 13 shows the results obtained in an experiment wherein the anti-cancer effect of anti-VISTA antibody clone V4 ([17] of Example 5) was compared to that of anti-PD-L1 antibody clone 10F.9G2 in a CT26 cell-line derived syngeneic mouse colon carcinoma model. The model was established by subcutaneous injection of 100,000 CT26 cells into the right flank of Balb/c mice (n=8 mice per treatment group).

V4 or anti-PD-L1 antibody were administered at 300 μg per dose every 3 days from day 3. A combination treatment of 300 μg V4+300 μg anti-PD-L1 antibody per dose was also included in the analysis.

Anti-VISTA antibody clone V4 was found to be highly potent in this model, and capable of inhibiting tumor growth by ˜60%.

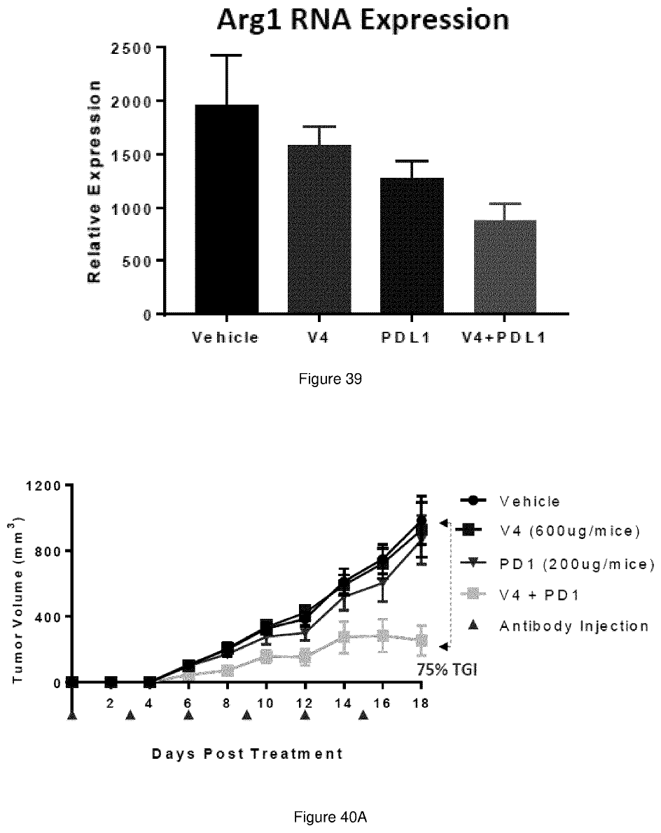

At day 21 tumors were harvested and evaluated for Arg1 RNA expression by RNA-seq analysis, according to the method described in Newman et al. Nat Methods. (2015) 12(5): 453-457. The results are shown in FIG. 39. Treatment with 4M2-C12 was associated with a significant reduction in Arg1 expression in tumors at day 21.

In another experiment a CT26 cell-line derived syngeneic mouse colon carcinoma model was established by subcutaneous injection of 100,000 CT26 cells into the right flank of Balb/c mice (n=8 mice per treatment group), and mic were treated by administration of 300 μg per dose every 3 days from day 3 of

an isotype control antibody, anti-PD-L1 antibody clone 10F.9G2, anti-VISTA antibody clone V4 ([17] of Example 5), anti-TIGIT antibody clone 1G9, anti-LAG-3 antibody clone C9B7W, anti-TIM-3 antibody clone RMT3-23, or combination treatments of 300 μg anti-PD-L1 antibody clone 10F.9G2 with 300 μg of each of the other of antibodies per dose was also included in the analysis.

The results of the calculated inhibition of tumor growth detected at day 15 are shown in FIG. 14. In this experiment anti-VISTA antibody clone V4 was found to be a more potent inhibitor of tumor growth than any other monotherapies directed against immune checkpoint molecules, and was found to perform better in combination with anti-PD-L1 therapy.

The inventors performed a further experiment in which CT26 tumors were established in the same way, and mice were then administered biweekly with 300 μg anti-VISTA antibody clone V4, 200 μg anti-PD-L1 antibody clone 10F.9G2, 300 μg anti-VISTA antibody clone V4+200 μg anti-PD-L1 antibody clone 10F.9G2, or PBS as a control condition. At the end of the experiment tumors were analysed by RNA-Seq to determine the relative numbers of MDSCs, CD8+ T cell and Tregs, according to the method described in Newman et al. Nat Methods. (2015) 12(5): 453-457 which is hereby incorporated by reference in its entirety.

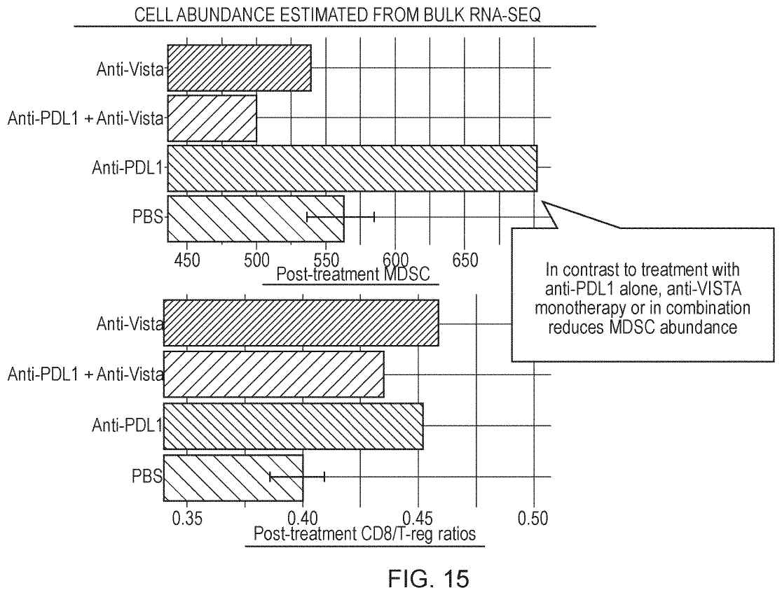

The results are shown in FIG. 15. Treatment with anti-VISTA antibody clone V4 (either alone or in combination with anti-PD-L1 treatment) was found to reduce the numbers of MDSCs, and to increase the CD8 T cell: Treg ratio. Analysis of changes in gene expression in the tumor microenvironment associated with anti-VISTA antibody treatment also revealed upregulation of expression of genes involved in phagocytic processes (e.g. actin filament-based movement), and downregulation of expression of arginase 1 (resulting in a less immunosuppressive environment).

In a further experiment, a CT26 cell-line derived syngeneic mouse colon carcinoma model was established in Balb/C mice as described above, and mice were administered from day 3 and every 3 days with: (i) 600 μg anti-VISTA antibody clone 13D5-1, (ii) 200 μg anti-PD-L1 antibody clone 10F.9G2, (iii) 600 μg anti-VISTA antibody clone 13D5-1+200 μg anti-PD-L1 antibody clone 10F.9G2, or (iv) an equal volume of PBS (as a negative control).

The results are shown in FIG. 22. Anti-VISTA antibody clone 13D5-1 (either alone or in combination with anti-PD-L1 treatment) was found to be able to inhibit tumor growth in this model.

### 5.2.2 LL2 Cell Model

The LL2 model was established by subcutaneous injection of 200,000 LL2 cells into the right flank of Balb/c mice (n=8 mice per treatment group), and mice were subsequently administered biweekly with 600 μg anti-VISTA antibody clone V4 ([17] of Example 5) or an equal volume of vehicle as a negative control.

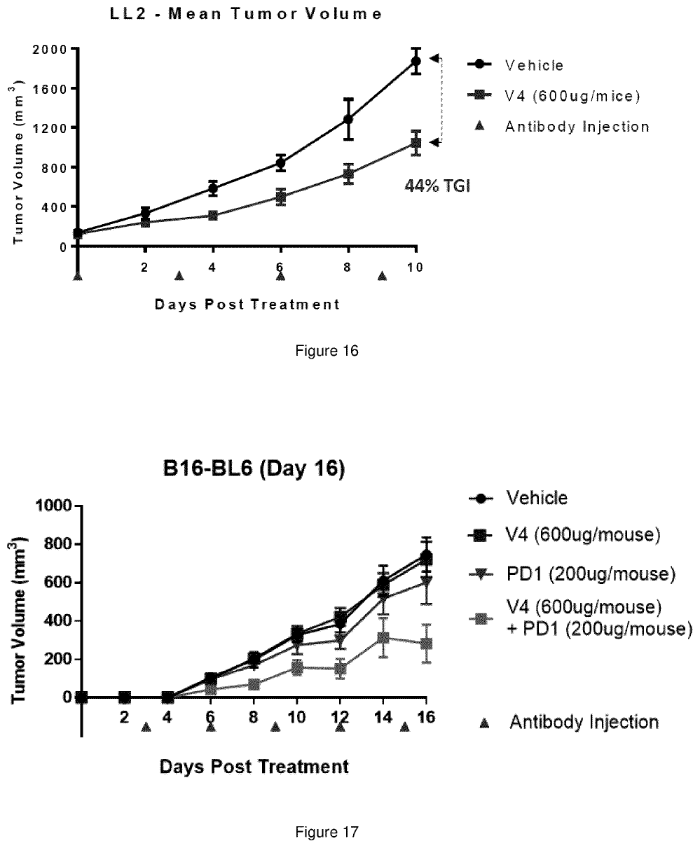

The results of the experiment are shown in FIG. 16. Anti-VISTA antibody clone V4 was found to be highly potent in this model-capable of inhibiting tumor growth by ˜44%.

### 5.2.3 B16-BL6 Cell Model

The B16-BL6 model was established by subcutaneous injection of 200,000 B16-BL6 cells into the right flank of C57BL/6 mice (n=8 mice per treatment group), and mice were subsequently administered biweekly (for a total of 6 doses) with 600 μg anti-VISTA antibody clone V4 ([17] of Example 5), 200 μg of anti-PD-1 antibody RMP1-14 (Bio X Cell), 600 μg anti-VISTA antibody clone V4+200 μg anti-PD-1 antibody, or an equal volume of vehicle as a negative control.

The results of the experiment are shown in FIG. 17. Anti-VISTA antibody clone V4 was found to be highly potent in combination with anti-PD-1 antibody treatment in this model.

### 5.2.4 4T1 Cell Model

The 4T1 cell-line derived syngeneic mouse mammary carcinoma model was established in Balb/c mice by subcutaneous injection of 250,000 4T1 cells into the right flank.

Mice were subsequently administered from day 3 and every 3 days (for a total of 6 doses) with either 300 or 600 μg of anti-VISTA antibody clone 13D5-1, isotype control antibody or an equal volume of vehicle as a negative control.

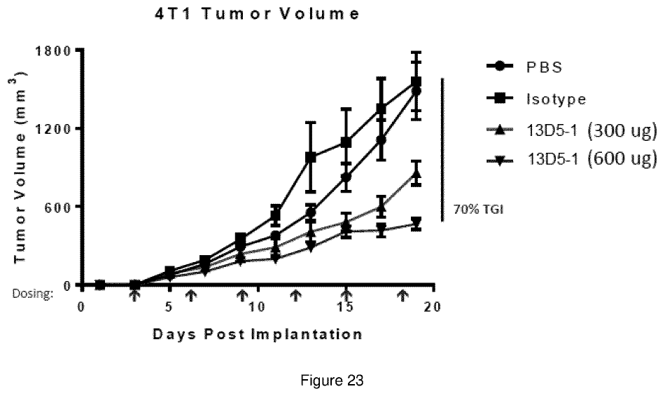

The results of the experiment are shown in FIG. 23. Anti-VISTA antibody clone 13D5-1 was found to be highly potent in this model-capable of inhibiting tumor growth by ˜70%.

### 5.3 Safety Pharmacology, Toxicology and Immunotoxicity

Anti-VISTA antibody clone V4 and humanized versions V4H1 and V4H2 were analysed in silico for safety and immunogenicity using IMGT DomainGapAlign (Ehrenmann et al., Nucleic Acids Res., 38, D301-307 (2010)) and IEDB deimmunization (Dhanda et al., Immunology. (2018) 153(1): 118-132) tools.

Anti-VISTA antibody clones V4H1 and V4H2 had sufficient homology to human heavy and light chains to be considered humanized (i.e. >85%), had numbers of potential immunogenic peptides few enough to be considered safe, and did not possess any other properties that could cause potential developability issues.

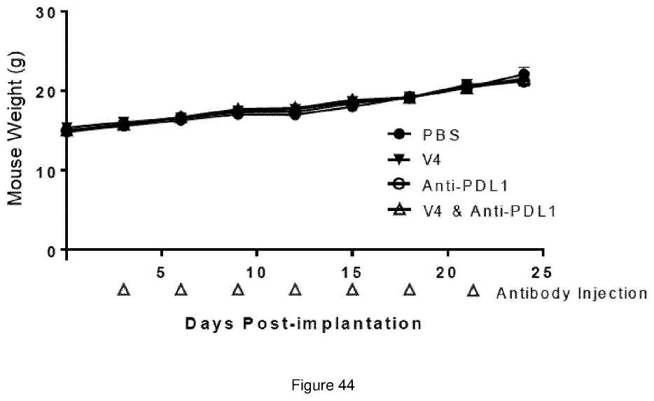

The inventors also weighed and analysed mice for signs of gross necroscopy during the course of the experiments described in Example 5.2; mice treated with anti-VISTA antibody clone V4 did not display any differences from PBS-treated control mice. FIG. 44 shows the results obtained during the course of the study described in Example 5.2.1.

The inventors further investigated hemotoxicity in an experiment in which mice were injected with a single dose of 900 μg anti-VISTA antibody clone V4 or an equal volume of PBS.

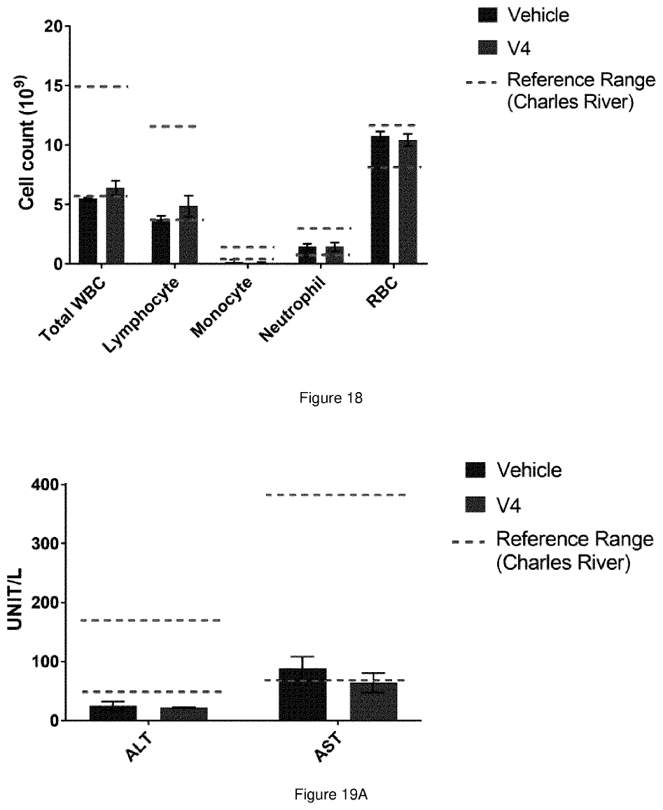

Blood samples were obtained and analysed for numbers of different types of white blood cells using HM5 Hematology Analyser. The results are shown in FIG. 18; the numbers of the different cell types were within the Charles River reference range and did not differ between the V4 and PBS-treated groups, and no differences in clinical signs, gross necroscopy or weight were detected between the different groups.

The mice were also analysed for correlates hepatotoxicity and nephrotoxicity, and the results are shown in FIG. 19. The levels detected were within the Charles River reference range and did not differ between the V4 and PBS-treated groups.

### 5.4 Treatment of Advanced Solid Tumors

### First in Human

Patients with advanced or metastatic solid tumors with disease progression or treatment intolerance after treatment with standard therapies and with adequate organ function and ECOG status are treated by intravenous injection of anti-VISTA antibody V4 ([1] of Example 2.2), V4H1 ([3] of Example 2.2) or V4H2 ([4] of Example 2.2), at a dose calculated in accordance with safety-adjusted ‘Minimal Anticipated Biological Effect Level’ (MABEL) approach. Patients are monitored for 28 days post-administration.

The patients are then evaluated according to the Common Terminology Criteria for Adverse Events (CTCAE), to determine the safety and tolerability of the treatment, and to determine the pharmacokinetics of the molecules.

Treatment with the anti-VISTA antibodies is found to be safe and tolerable.

### Dose Escalation—Monotherapy

Patients with advanced or metastatic solid tumors with disease progression or treatment intolerance after treatment with standard therapies and with adequate organ function and ECOG status (n=18-24) are treated by intravenous injection of anti-VISTA antibody V4 ([1] of Example 2.2), V4H1 ([3] of Example 2.2) or V4H2 ([4] of Example 2.2), in accordance with a 3+3 model based escalation with overdose control (EWOC) dose escalation.

The patients are then evaluated according to the Common Terminology Criteria for Adverse Events (CTCAE) to determine the safety and tolerability of the treatment, and the pharmacokinetics of the molecules and efficacy of the treatment is evaluated. The maximum tolerated dose (MTD) and maximum administered dose (MAD) are also determined.

### Dose Escalation—Combination Therapy

Patients with advanced or metastatic solid tumors with disease progression or treatment intolerance after treatment with standard therapies and with adequate organ function and ECOG status (n=9) are treated with anti-VISTA antibody V4 ([1] of Example 2.2), V4H1 ([3] of Example 2.2) or V4H2 ([4] of Example 2.2), in accordance with a 3+3 model based escalation with anti-PD-1 or anti-PD-L1 antibody (3 mg/kg).

The patients are then evaluated according to the Common Terminology Criteria for Adverse Events (CTCAE) to determine the safety and tolerability of the treatment, and the pharmacokinetics of the molecules and efficacy of the treatment is evaluated.

### Dose Expansion

Treated patients are analysed for overall response rate, expression of tumor markers, circulating tumor cells, progression-free survival, overall survival, safety and tolerability.

The anti-VISTA antibodies are found to be safe and tolerable, to be able to reduce the number/proportion of cancer cells, reduce tumor cell marker expression, increase progression-free survival and increase overall survival.

### 5.5 Treatment of Lymphoma

### First in Human

Patients with lymphoma (NHL and HL) who did not benefit from 1 line of chemotherapy, who have not received allogeneic stem cell transplantation and are likely to respond to rituximab (NHL) and nivolumab or pembrolizumab (HL) are treated by intravenous injection of anti-VISTA antibody V4 ([1] of Example 2.2), V4H1 ([3] of Example 2.2) or V4H2 ([4] of Example 2.2), at a dose calculated in accordance with safety-adjusted ‘Minimal Anticipated Biological Effect Level’ (MABEL) approach. Patients are monitored for 28 days post-administration.

The patients are then evaluated according to the Common Terminology Criteria for Adverse Events (CTCAE), to determine the safety and tolerability of the treatment, and to determine the pharmacokinetics of the molecules.

Treatment with the anti-VISTA antibodies is found to be safe and tolerable.

### Dose Escalation—Monotherapy

Patients with lymphoma (NHL and HL) who did not benefit from 1 line of chemotherapy, who have not received allogeneic stem cell transplantation and are likely to respond to rituximab (NHL) and nivolumab or pembrolizumab (HL) are treated by intravenous injection of anti-VISTA antibody V4 ([1] of Example 2.2), V4H1 ([3] of Example 2.2) or V4H2 ([4] of Example 2.2), in accordance with a 3+3 model based escalation with overdose control (EWOC) dose escalation.

The patients are then evaluated according to the Common Terminology Criteria for Adverse Events (CTCAE) to determine the safety and tolerability of the treatment, and the pharmacokinetics of the molecules and efficacy of the treatment is evaluated. The maximum tolerated dose (MTD) and maximum administered dose (MAD) are also determined.

### Dose Escalation—Combination Therapy

Patients with lymphoma (NHL and HL) who did not benefit from 1 line of chemotherapy, who have not received allogeneic stem cell transplantation and are likely to respond to rituximab (NHL) and nivolumab or pembrolizumab (HL) are treated by intravenous injection of anti-VISTA antibody V4 ([1] of Example 2.2), V4H1 ([3] of Example 2.2) or V4H2 ([4] of Example 2.2), in accordance with a 3+3 model based escalation with anti-PD-L1 antibody.

The patients are then evaluated according to the Common Terminology Criteria for Adverse Events (CTCAE) to determine the safety and tolerability of the treatment, and the pharmacokinetics of the molecules and efficacy of the treatment is evaluated.

### Dose Expansion

Treated patients are analysed for overall response rate, expression of cancer cell markers, circulating cancer cells, progression-free survival, overall survival, safety and tolerability.

The anti-VISTA antibodies are found to be safe and tolerable, to be able to reduce the number/proportion of cancer cells, reduce tumor cell marker expression, increase progression-free survival and increase overall survival.

### Example 6: Production and Characterisation of VISTA-Binding Antibodies Comprising Different Fc Regions

### 6.1 Production and Characterisation of VISTA-Binding Antibodies Comprising Different Fc Regions

4M2-C12 was produced in mouse IgG2a LALA PG format. The molecule is a heteromer of the heavy chain polypeptide having the sequence shown in SEQ ID NO:249, and the light chain polypeptide having the sequence shown in SEQ ID NO:250. The heavy chain sequence comprises leucine (L) to alanine (A) substitutions in the CH2 region, at positions 4 and 5 numbered according to SEQ ID NO:253, and a proline (P) to glycine (G) substitution at position 99 numbered according to SEQ ID NO:253. These substitutions are referred to in the literature as L234A, L235A and P329G, and are described in mouse IgG2a Fc e.g. in Lo et al. J. Biol. Chem (2017) 292(9): 3900-3908, which is hereby incorporated by reference in its entirety. 4M2-C12 mIgG2a LALA PG was produced by co-expression of nucleic acids encoding the heavy and light chain polypeptides in CHO cells, and was subsequently purified.

| Antigen- | | |
|---|---|---|
| biding | | |
| molecule | Polypeptides | Antibody |
| [18] | 4M2-C12 mlgG2a LALA PG HC (SEQ ID | 4M2-C12 mIgG2a LALA PG |
| | NO: 249) | |
| | + | |
| | 4M2-C12 CL (SEQ ID NO: 250) | |

4M2-C12 was also produced in mouse IgG2a NQ format. The molecule is a heteromer of the heavy chain polypeptide having the sequence shown in SEQ ID NO:258, and the light chain polypeptide having the sequence shown in SEQ ID NO:250. The heavy chain sequence comprises an asparagine (N) to glutamine (Q) substitution in the CH2 region, at position 67 according to SEQ ID NO:253. This substitution is referred to in the literature as N297Q, and is described in mouse IgG2a Fc e.g. in Lo et al. J. Biol. Chem (2017) 292(9): 3900-3908. 4M2-C12 mIgG2a NQ was produced by co-expression of nucleic acids encoding the heavy and light chain polypeptides in CHO cells, and was subsequently purified.

| Antigen- | | |
|---|---|---|
| biding | | |
| molecule | Polypeptides | Antibody |
| [19] | 4M2-C12 mIgG2a NQ HC (SEQ ID NO: 258) | 4M2-C12 mIgG2a NQ |
| | + | |
| | 4M2-C12 CL (SEQ ID NO: 250) | |

4M2-C12 was produced in mouse IgG1 format. The molecule is a heteromer of the heavy chain polypeptide having the sequence shown in SEQ ID NO:266, and the light chain polypeptide having the sequence shown in SEQ ID NO:250. 4M2-C12 mIgG1 was produced by co-expression of nucleic acids encoding the heavy and light chains polypeptides in CHO cells, and was subsequently purified.

| Antigen- | | |
|---|---|---|
| biding | | |
| molecule | Polypeptides | Antibody |
| [20] | 4M2-C12 mIgG1 HC (SEQ ID NO: 266) | 4M2-C12 mIgG1 |
| | + | |
| | 4M2-C12 CL (SEQ ID NO: 250) | |

### 6.2 Analysis of Binding of VISTA-Binding Antibodies Comprising Different Fc Regions to Fc Receptors

Binding of 4M2-C12 in different antibody formats to human, cynomolgus and murine VISTA protein was assessed via ELISA, and binding to mouse Fcγ receptors and mouse FcRn was assessed by BLI using a Pall ForteBio Octet QK 384 system.

Histidine-tagged mFcγRIV (50036-M08H), mFcγRIII (50326-M08H), mFcγRIIb (50030-M08H), and mFcRn (CT009-H08H) were obtained from Sino Biological. Anti-Penta-HIS (HIS1K) biosensors were purchased from Forte Bio (18-5120).

For the kinetic experiment, anti-Penta-HIS biosensors were incubated for 60 sec in PBS buffer (pH 7.2) to obtain the first baseline, and were subsequently loaded for 120 sec with 200 nM mFcγRIV (orthologue of hFcγRIIIa), 160 nM mFcγRIII (orthologue of hFcγRIIa), 75 nM mFcγRIIb (orthologue of hFcγRIIb) or 120 nM mFcRn in PBS (pH 7.2). After loading, biosensors were incubated for 60 sec in PBS buffer (pH 7.2 for Fcγ receptors and pH 5.8 for FcRn) to obtain the second baseline, and for 60 sec with a 6 point 2-fold dilution series of the test antibodies (2000 nM-62.5 nM for mFcγ receptor binding, and 500 nM-15.6 nM for FcRn binding) in PBS (pH 7.2 for Fcγ receptors and pH 5.8 for FcRn) to obtain the association curves. Finally, the biosensors were incubated for 120 sec in PBS (pH 7.2 for mFcγR and pH 5.8 for mFcRn) to obtain the dissociation curves. Kinetic and affinity constants were calculated by global fitting of the association and dissociation data to a 1:1 binding model.

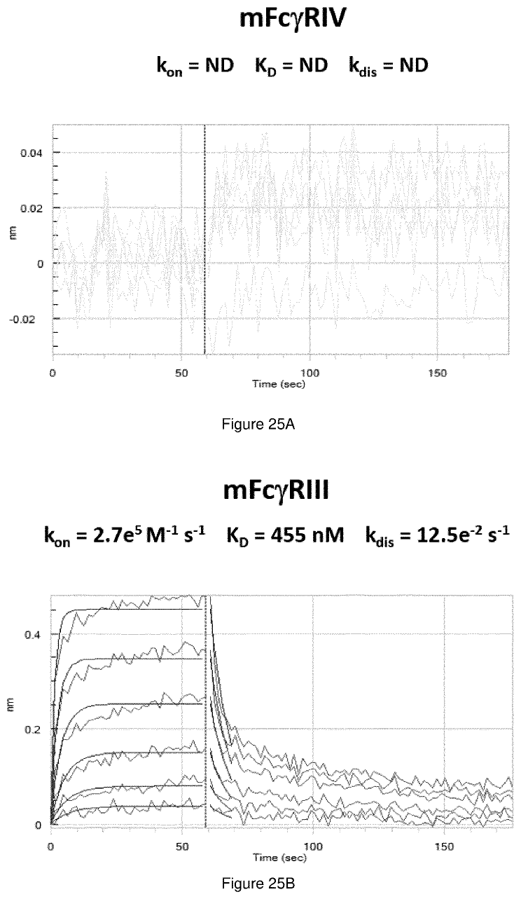

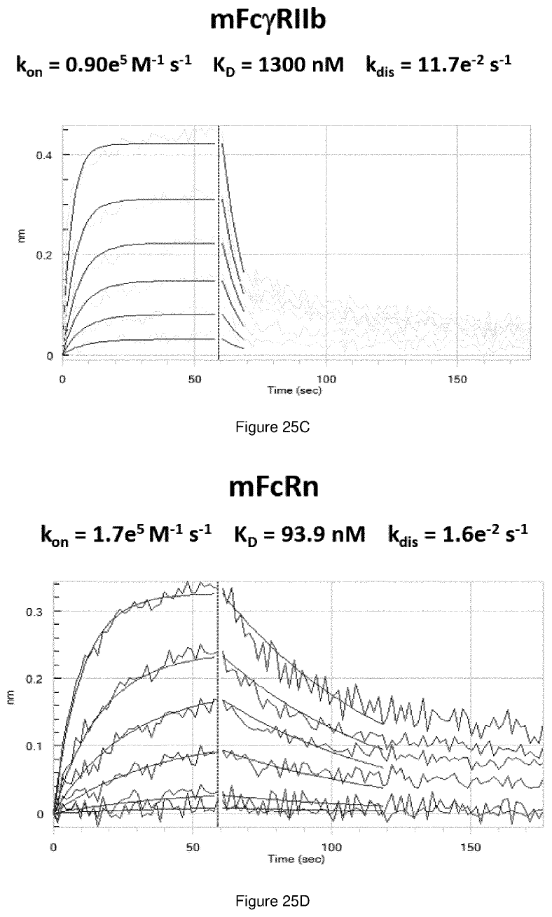

The results for analysis of binding of 4M2-C12-mIgG1 to different mouse Fcγ receptors and mouse FcRn are shown in FIGS. 25A to 25D.

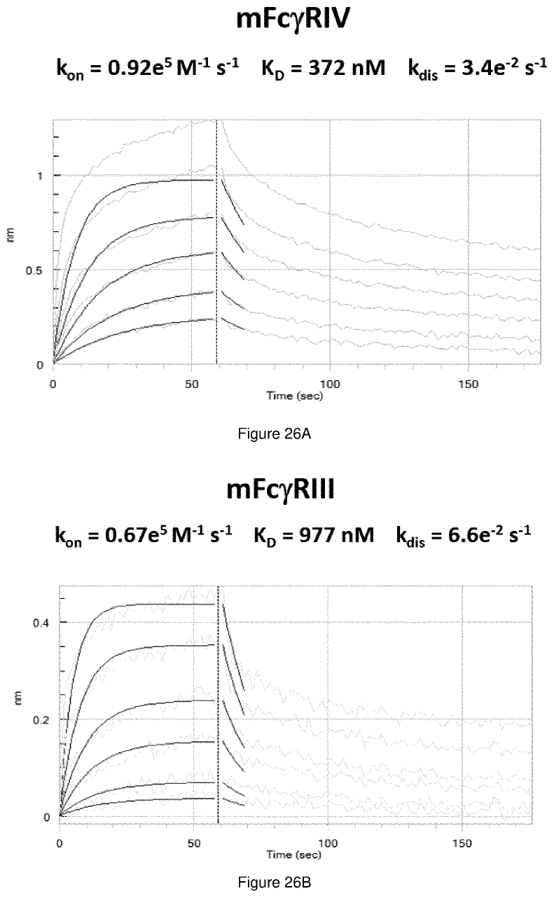

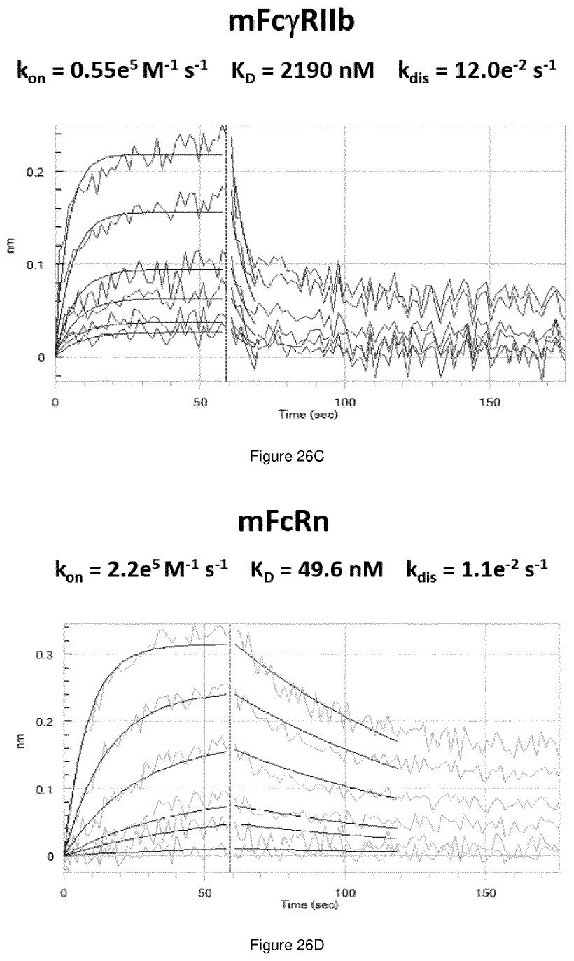

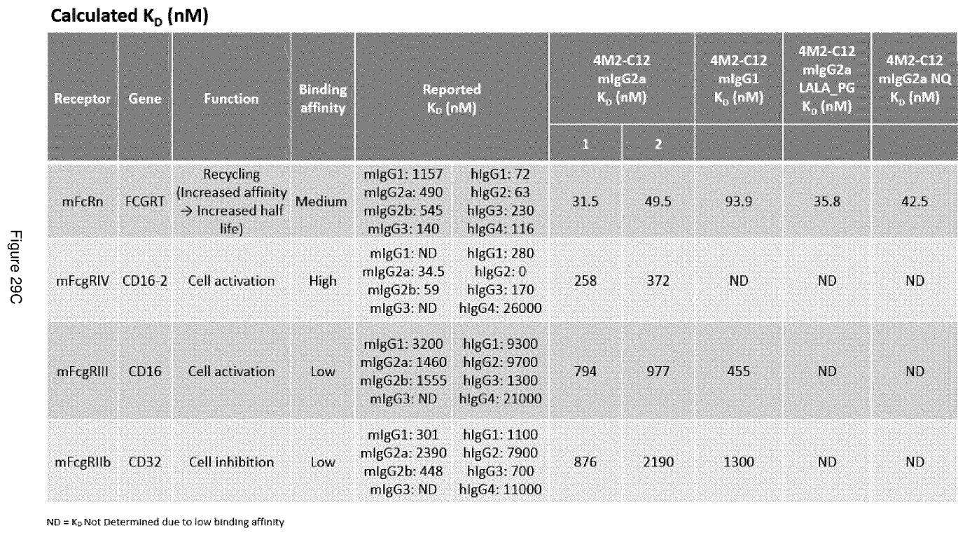

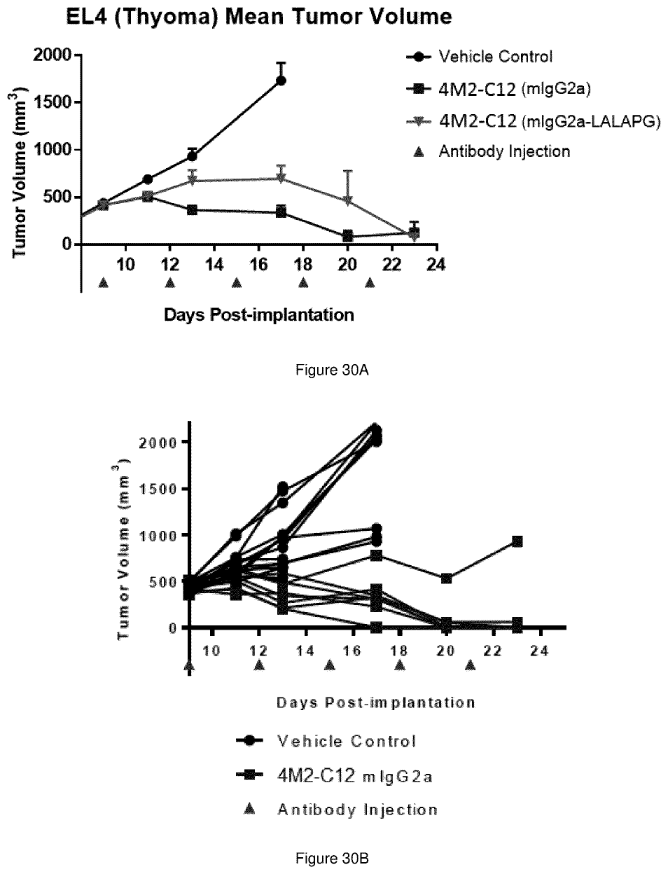

The results for analysis of binding of 4M2-C12-mIgG2a to different mouse Fcγ receptors and mouse FcRn are shown in FIGS. 26A to 26D. Two separate experiments were performed (1 and 2; see FIGS. 29A to 29C); FIGS. 26A to 26D show the results obtained in experiment 2. The level of binding detected for the different Fcγ receptors was comparable to the level reported in the scientific literature.

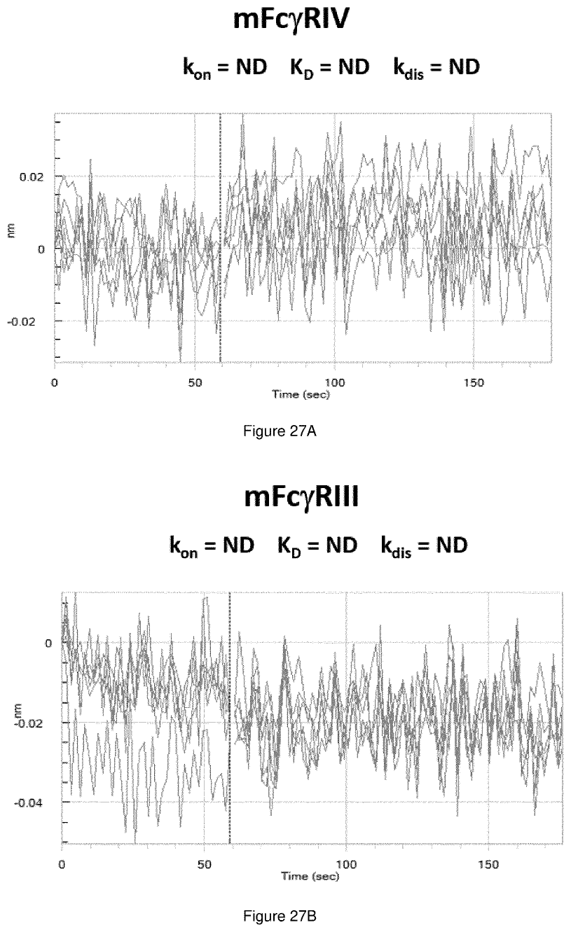

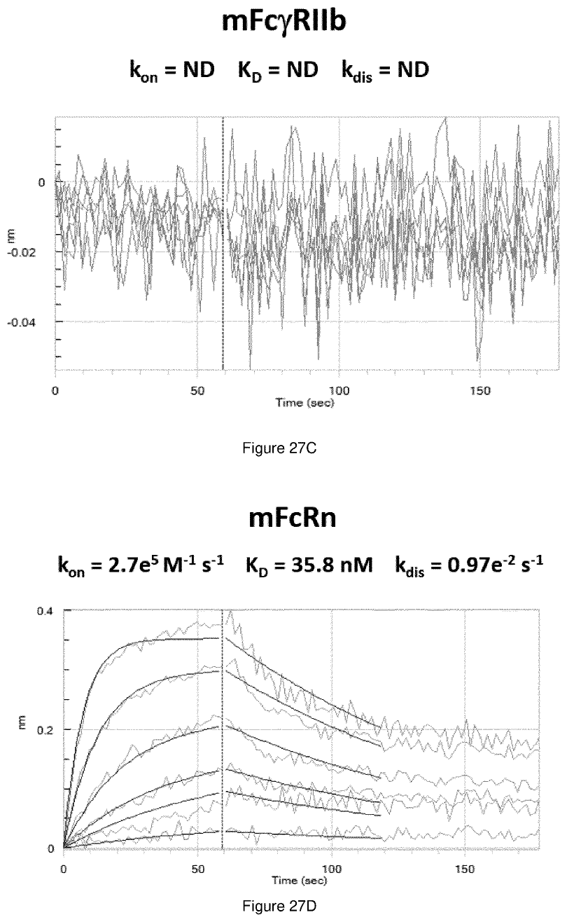

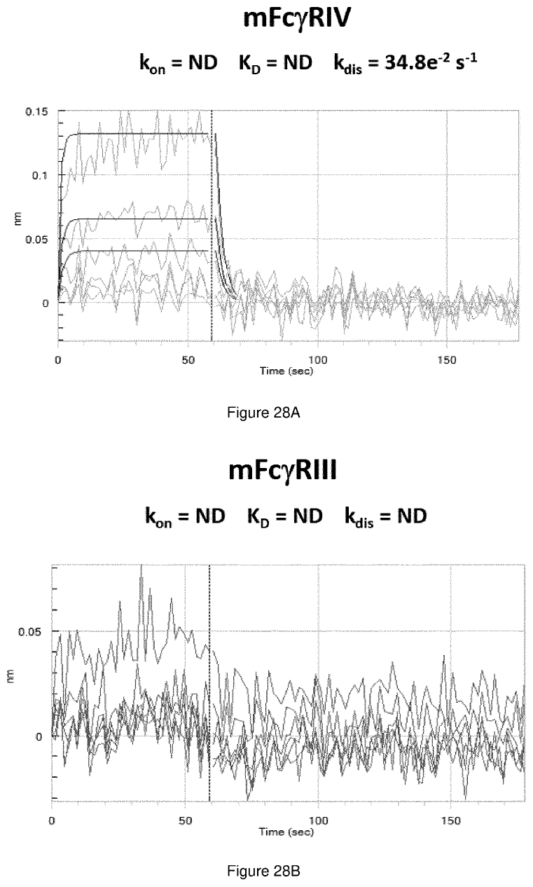

The results for analysis of binding of 4M2-C12-mIgG2a LALA PG to different mouse Fcγ receptors and mouse FcRn are shown in FIGS. 27A to 27D. The level of binding to mFcγRIV, mFcγRIII and mFcγRIIb was negligible/undetectable, and the level of binding to mFcRn was similar to the level of binding to mFcRn by 4M2-C12-mIgG2a.

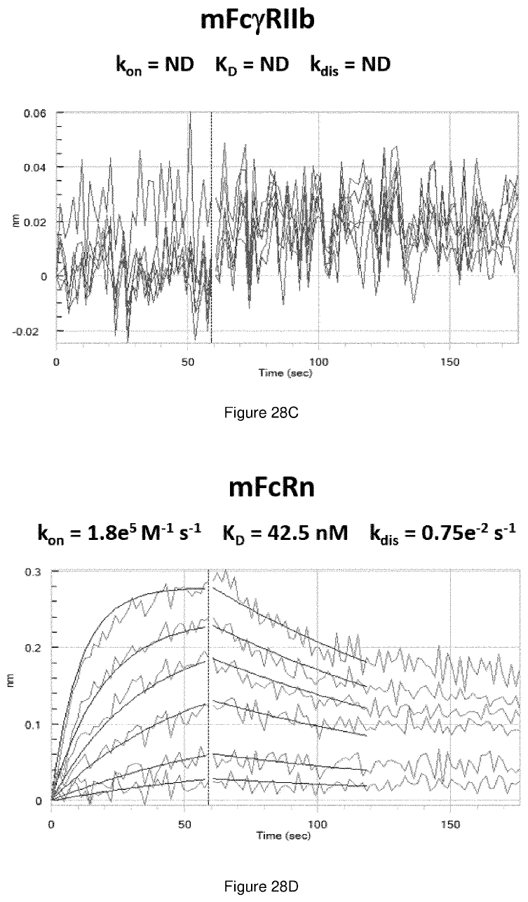

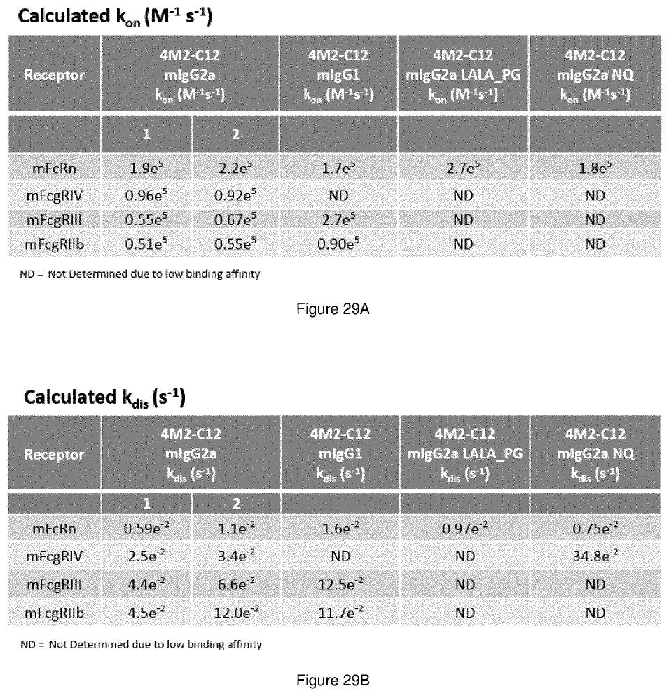

The results for analysis of binding of 4M2-C12-mIgG2a NQ to different mouse Fcγ receptors and mouse FcRn are shown in FIGS. 28A to 28D.

The Kₒₙ, K_{dis} and K_{D} values for binding to different Fc receptors determined for 4M2-C12-mIgG1, 4M2-C12-mIgG2a, 4M2-C12-mIgG2a LALA PG and 4M2-C12-mIgG2a NQ are summarised in the tables of FIGS. 29A to 29C.

### Example 7: Analysis In Vivo of VISTA-Binding Antibodies Comprising Different Fc Regions

4M2-C12-mIgG2a and 4M2-C12-mIgG2a LALA PG (see Example 6.1) were evaluated for efficacy to treat cancer in vivo in a syngeneic EL4 T-cell leukemia/lymphoma model.

EL4 cells cultured in DMEM supplemented with 10% Horse serum (FBS) and 1% Pen/Strep. Cells were cultured at 37° C. in a 5% CO₂ incubator.

C57BL/6 mice, approximately 6 weeks old were obtained from InVivos (Singapore). Animals were housed under specific pathogen-free conditions and were treated in compliance with the Institutional Animal Care and Use Committee (IACUC) guidelines. C57BL/6 mice were inoculated with 2×10⁵ EL4 T-cell leukemia/lymphoma cells on the right flank. Post tumor implantation, when tumors reached 350 to 400 mm³ in size, mice were randomized to the following treatment groups: a) vehicle control (PBS), b) 4M2-C12 mIgG2a ([17] of Example 5), or c) 4M2-C12 mIgG2a-LALA PG ([18] of Example 6), at a dose of 25 mg/kg. The treatments were administered intraperitoneally every 3 days for a total of 5 doses.

Tumor volume was measured 3 times a week using a digital caliper, and calculated using the formula [L×W2/2]. Study End point was considered to have been reached once the tumors of the vehicle control treatment group measured >1.5 cm in length.

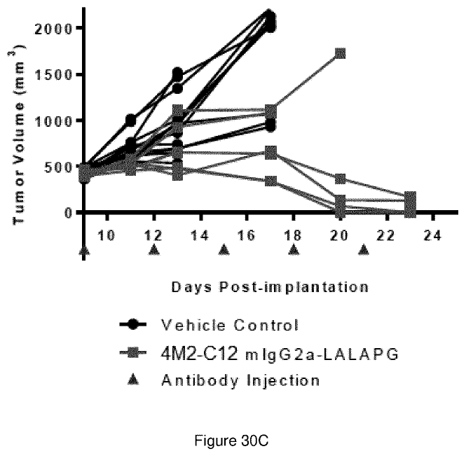

The results are shown in FIGS. 30A to 30C. By the final day of the study (Day 23 post implantation), the mean tumor volume in both the 4M2-C12 IgG2a and 4M2-C12 IgG2a LALA PG treatment groups was below the size for reliable measurement (<30 mm³). By contrast, the average tumor volume in mice in the vehicle (PBS) treatment group exceeded 2000 mm³ by Day 18 and all animals were euthanized.

Thus 4M2-C12 was found to display potent inhibition of tumor growth in both IgG2a and IgG2a LALA PG formats.

Treatment of highly established EL4 tumor-bearing mice with anti-VISTA antibody 4M2-C12 was found to be very effective as a monotherapy, and resulted in tumor clearance.

The biological activity of 4M2-C12 was found not to be dependent on engagement of murine Fcγ receptors, strongly suggesting that 4M2-C12 exerts its biological activity through inhibition of VISTA-mediated signalling.

### Example 8: Analysis of the Epitope of VISTA Bound by the Antibodies

Anti-VISTA antibodies were evaluated to determine whether they compete with one another for binding to VISTA.

VSTB112 has previously been suggested to bind VISTA in several regions. The major epitopes have been proposed to correspond to positions 59 to 68 and positions 86 to 97 of SEQ ID NO:1 (i.e. SEQ ID NOs: 271 and 272). The minor epitopes have been proposed to correspond to positions 71 to 84 and positions 150 to 166 of SEQ ID NO:1 (i.e. SEQ ID NOs: 273 and 274); see e.g. WO 2017/137830 A1, e.g. at paragraph [0302]. VSTB112 is described e.g. in WO 2015/097536 A2, which is hereby incorporated by reference in its entirety.

IGN175A is thought to bind to VISTA within the first 32 amino acids of the mature protein (i.e. within positions 33 to 64 of SEQ ID NO:1 (i.e. SEQ ID NO:275)). IGN175A is described e.g. in WO 2014/197849 A2, which is hereby incorporated by reference in its entirety.

Epitope binning experiments were performed by BLI using the Octet QK384 system (ForteBio). Briefly, human VISTA-His recombinant protein in PBS (4.7 μg/ml) was immobilized to Anti-Penta His sensor (HIS1K, ForteBio), for 5 mins. Baseline signals in PBS were measured for 30s before loading of 400 nM saturating antibody in PBS for 10 mins, and at a shake speed of 1000 rpm, followed by a 120 s dissociation step using PBS. Biosensors were subsequently treated with 300 nM competing antibody in PBS for 5 mins, at a shake speed of 1000 rpm, followed by a 120 s dissociation step using PBS.

The following antigen-binding molecules were analysed in the experiment:
4M2-C12 (V4) in IgG1 format ([1] of Example 2.2)
A humanised and affinity-matured variant of 4M2-C12 (V4-C1) in IgG1 format ([21] of Example 13)
IGN175A IgG1 (comprising IGN175A HC (SEQ ID NO: 267)+IGN175A LC (SEQ ID NO: 268))
VSTB112 IgG1 (comprising VSTB112 HC (SEQ ID NO: 269)+VSTB112 LC (SEQ ID NO: 270))

The following antigen/saturating antibody/competing antibody combinations were investigated:

| Antigen | Saturating Antibody | Competing Antibody |
|---|---|---|
| human VISTA | V4-C1 IgG1 | IGN175A IgG1 |
| human VISTA | V4-C1 IgG1 | V4-C1 IgG1 |
| human VISTA | V4-C1 IgG1 | VSTB112 IgG1 |
| human VISTA | VSTB112 IgG1 | IGN175A IgG1 |
| human VISTA | IGN175A IgG1 | V4-C1 IgG1 |
| human VISTA | VSTB112 IgG1 | V4-C1 IgG1 |
| human VISTA | IGN175A IgG1 | IGN175A IgG1 |
| None (PBS) | V4-C1 IgG1 | IGN175A IgG1 |

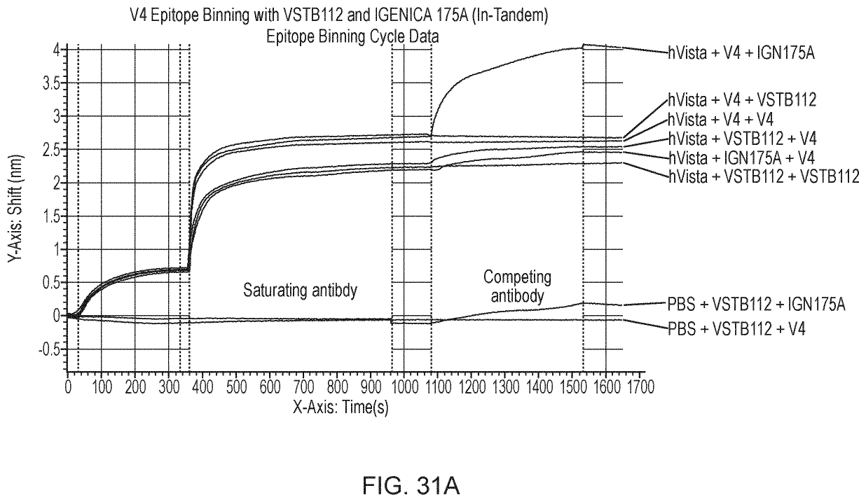

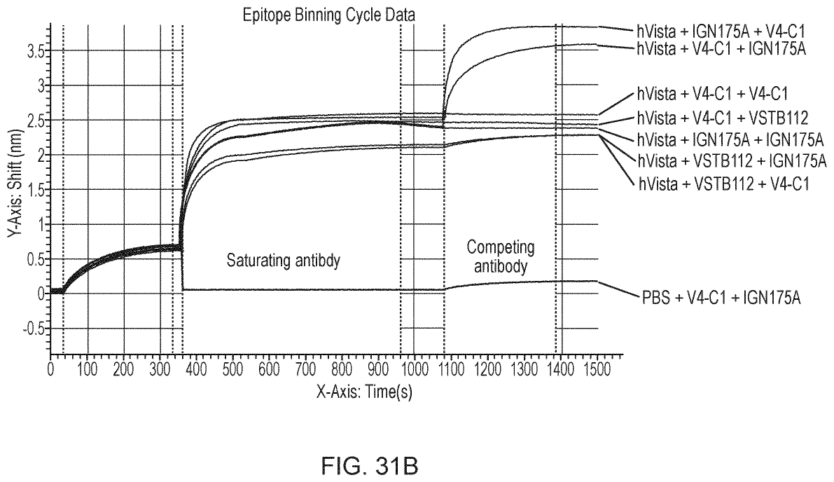

The results are shown in FIGS. 31A and 31B.

V4 and V4-C1 IgG1 were found not to compete with IGN175A for binding to VISTA. VSTB112 was found to partially compete with V4, V4-C1 and IGN175A for binding to VISTA. Changes in response (in nm) upon addition of the competing antibody are shown below.

| | | Competing Antibody | | |
|---|---|---|---|---|
| | | IGN175A | V4-C1 IgG1 | VSTB112 |
| Saturating | IGN175A | 0.0454 | 1.4362 | — |
| Antibody | V4-C1 IgG1 | 1.0661 | −0.0158 | −0.0124 |
| | VSTB112 | 0.1392 | 0.1579 | — |

The results indicate that 4M2-C12 and IGN175A bind to topically distant regions of VISTA, and that VSTB112 binds to VISTA in regions which are proximal to 4M2-C12 and IGN175A.

The fact that V1-C1 and IGN175A do not compete for binding to VISTA taken together with the observation that VSTB112 competes with IGN175A for binding to VISTA indicates that antibodies comprising the CDRs of 4M2-C12 bind to an epitope of VISTA which is non-identical to the epitope of VISTA bound by IGN175A, and which is also non-identical to the epitope of VISTA bound by VSTB112.

From analysis of the sequence for VISTA, the immunogen used to raise 4M2-C12 and species cross-reactivity data, the inventors concluded that 4M2-C12 and derivatives thereof bind to the sequence shown in SEQ ID NO:322 (which corresponds to positions 76 to 81 of SEQ ID NO:1).

### Example 9: Analysis of the Ability of VISTA-Binding Antibodies to Rescue VISTA-Mediated Inhibition of T Cell Proliferation

The ability of anti-VISTA antibodies to rescue the inhibitory effects of VISTA-mediated signalling was analysed in an in vitro assay.

Briefly, 96-well plates were coated with anti-CD3 and VISTA-Ig or control-Ig at concentration ratios of either 1:1 (2.5 μg/ml anti-CD3: 2.5 μg/ml of VISTA/control Ig) or 2:1 (2.5 μg/ml anti-CD3: 1.25 μg/ml VISTA/control Ig). Irrelevant antigen-Ig was used as control condition. Plates were incubated overnight at 4° C.

PBMCs were purified from freshly collected blood samples and further enriched for T cells using human Pan T Cell Isolated Kit (Miltenyi Biotec). The enriched T cell populations were then labelled with CFSE.

Wells were washed three times with PBS, and 100,000 CFSE-labelled T cells were added to each well, in complete RPMI 1640 medium supplemented with 10% FBS, in the presence of 4M2-C12 IgG1 ([1] of Example 2.2) at a final concentration of 20 μg/ml or 50 μg/ml, or in the presence of VSTB 1 12 at a final concentration of 20 μg/ml, or in the absence of added antibody.

After 5 days, cells were harvested, labelled with fluorescently conjugated anti-CD4 and anti-CD8 antibodies, and analysed by flow cytometry using a Macsquant Analyzer 10.

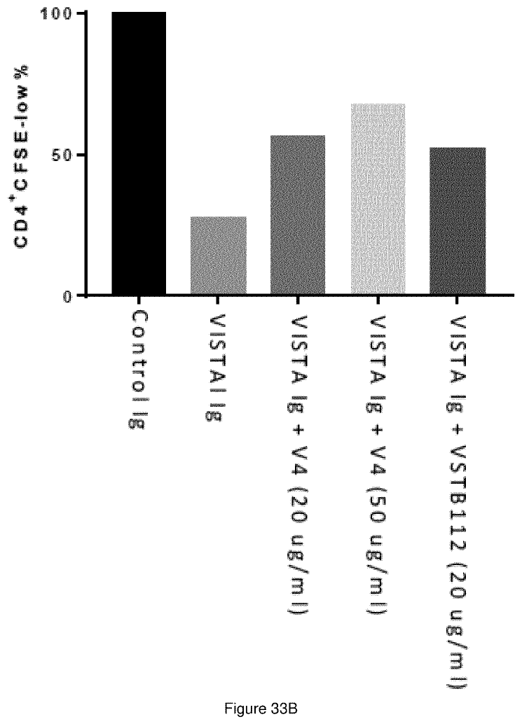

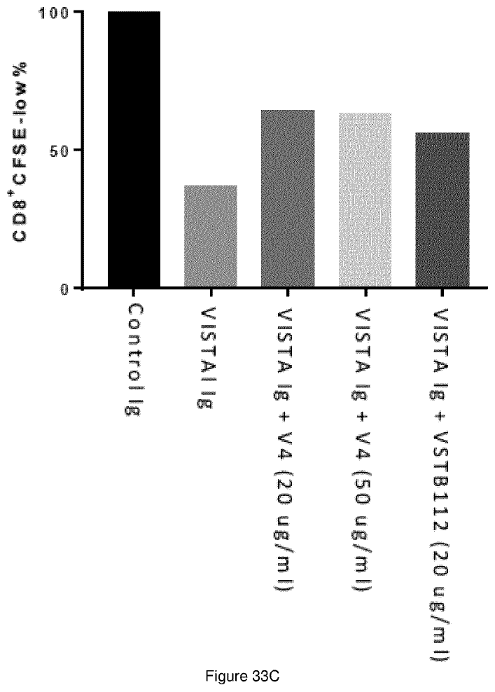

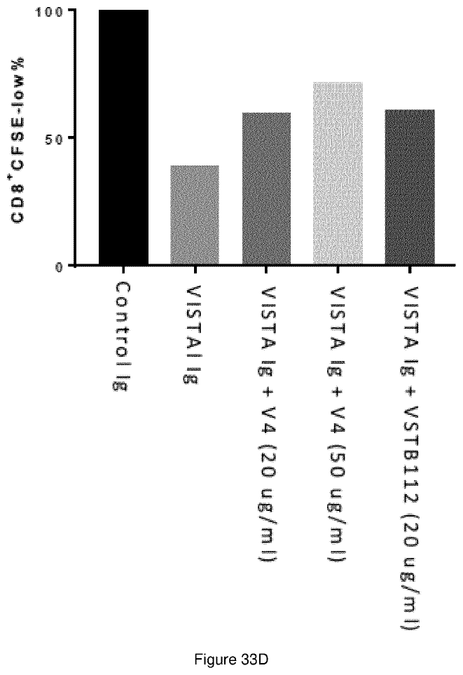

The results of the experiments are shown in FIGS. 33A to 33D. 4M2-C12 was found to restore the ability of both CD4+ T cells and CD8+ T cells to proliferate.

Importantly, 4M2-C12 was found to be more effective at restoring proliferation of T cells than VSTB112.

### Example 10: Analysis of the Ability of VISTA-Binding Antibodies to Promote Production of IL-6 by THP1 Cells in Response to LPS

The ability of anti-VISTA antibodies to promote production of IL-6 by THP-1 cells in response to LPS stimulation was analysed in an in vitro assay.

Briefly, undifferentiated THP1 cells were seeded in 96 well plates in duplicate (100,000 cells/well), in RPMI media without FBS or pen/strep. Cells subsequently treated with LPS (final concentration of 100 μg/ml) and MnCl₂ (100 μM), in the presence of different concentrations of 4M2-C12 IgG1 ([1] of Example 2.2) ranging from 2000 μg/ml to 7.8 μg/ml, or different concentrations of VSTB112 ranging from 1000 μg/ml to 7.8 μg/ml. After 3 days, the cell culture supernatant was collected and analysed by ELISA to determine the level of IL-6, using the IL-6 Human ELISA Kit (Invitrogen).

The results are shown in FIGS. 34A and 34B. 4M2-C12 was found to promote the production of more IL-6 by LPS-stimulated THP1 cells than VSTB112.

### Example 11: Analysis of the Ability of VISTA-Binding Antibodies to Promote Production of IL-6 In Vivo

IL-6 production in response to treatment with 4M2-C12 was investigated in vivo.

Briefly, C57BL/6 mice (n=3) were administered with a single 600 μg dose of 4M2-C12 mIgG2a ([17] of Example 5), and blood samples were harvested from mice at 2 hr before administration, and 0.5 hr, 6 hr, 24 hr, 96 hr, 168 hr and 336 hr post-administration.

The serum was analysed for IL-6 content using the Mouse IL-6 ELISA Kit (Abcam, ab100712).

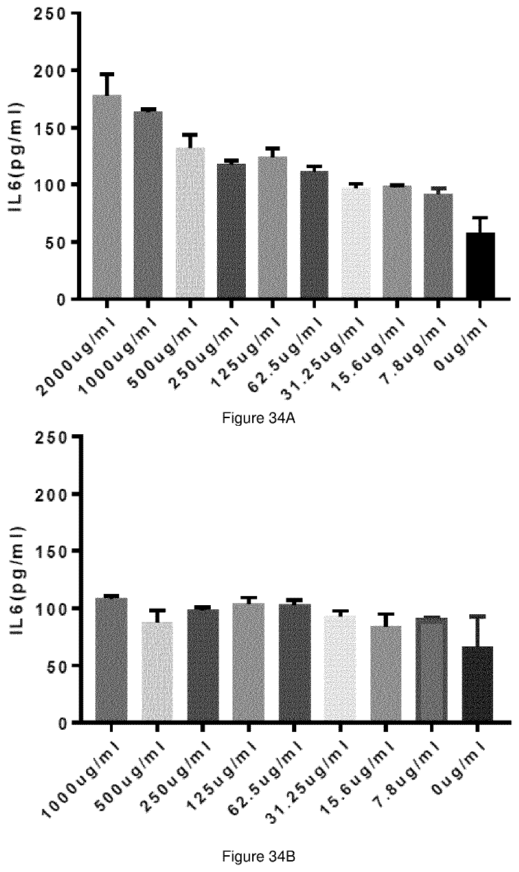

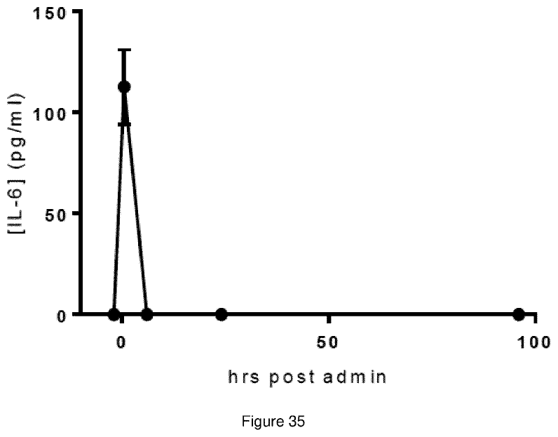

The results are shown in FIG. 35. IL-6 was detected in the serum at 0.5 hr after administration of 4M2-C12 mIgG2a.

### Example 12: Analysis In Vivo of VISTA-Binding Antibodies Alone or in Combination with Anti-PD-1/PD-L1 Antibody

### 12.1 CT26 Cell Model

A syngeneic model of T cell leukemia/lymphoma was generated by injecting 1×10⁵ CT26 cells subcutaneously into the right flank of Balb/c mice.

Mice (7 per treatment group) were administered intraperitoneally every 3 days for a total of 7 doses with:
600 μg of 4M2-C12 IgG2a ([17] of Example 5)
200 μg of anti-PD-1 antibody (clone RMP1-14 (Bioxcell))
600 μg of 4M2-C12 IgG2a+200 μg of anti-PD-1 antibody
PBS only

Tumor volume was measured 3 times a week using a digital caliper and calculated using the formula [L×W2/2]. Study End point was considered to have been reached once the tumors of the control arm measured >1.5 cm in length.

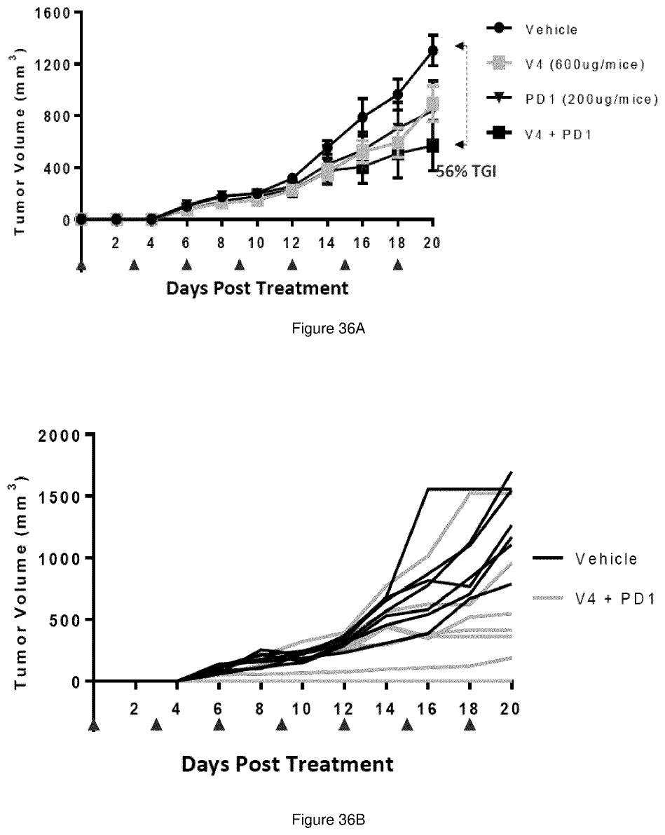

The results are shown in FIGS. 36A and 36B. Combination therapy with anti-VISTA antibody 4M2-C12 and anti-PD-1 inhibited tumor growth to a greater extent than either agent used alone.

Immunoprofiling of the tumor-infiltrating CD45+ cells was undertaken. Briefly, at day 22 of the experiment tumors were harvested, processed into single cell suspensions and stained with antibodies specific for immune cell surface proteins (CD45, CD4, CD8, CD25, CD11b, Ly6G, and Ly6C).

Samples were analysed by flow cytometry, and were classified into the following immune cell subsets based on their staining for the different immune cell surface proteins as follows:
CD4 cells: CD45⁺CD4⁺;
CD8 T cells: CD45⁺CD8⁺;
Treg cells: CD45⁺CD4⁺CD25⁺;
Granulocytic MDSC (g-MDSC): CD45⁺CD11b⁺Ly6G⁺Ly6C^{lo/-}.
Monocytic MDSC (m-MDSC): CD45⁺CD11b⁺Ly6G⁻Ly6C^{hi/+}

The percentage of tumor-infiltrating CD45+ cells having the indicated phenotypes are summarised below:

| | CD4 | CD8 | | | |
|---|---|---|---|---|---|
| Treatment Group | cells | cells | Treg | g-MDSC | m-MDSC |
| PBS | 1.03% | 5.99% | 0.09% | 33.08% | 9.8% |
| 4M2-C12 IgG2a | 1.66% | 6.51% | 0.11% | 26.4% | 8.34% |
| anti-PD-1 antibody | 1.05% | 7.21% | 0.14% | 46.2% | 15.25% |
| 4M2-C12 IgG2a + | 1.55% | 9.98% | 0.22% | 15.66% | 14.04% |
| anti-PD-1 antibody | | | | | |

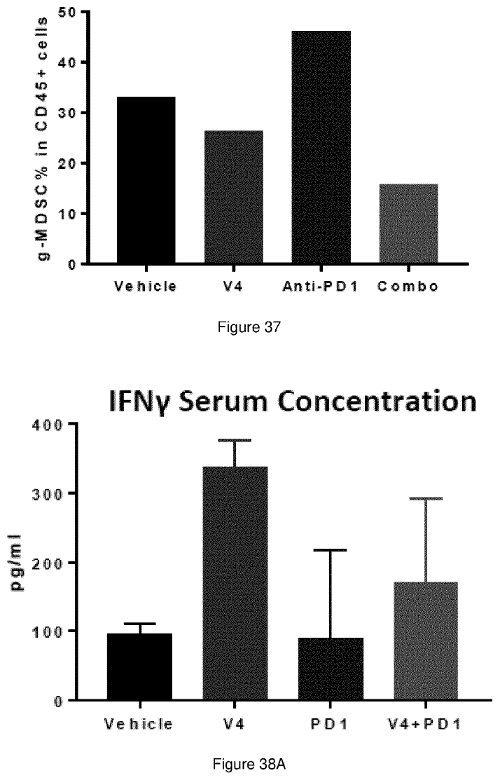

The percentage of tumor-infiltrating CD45+ cells which were g-MDSC is shown in FIG. 37. Treatment with 4M2-C12 (either alone, or in combination with anti-PD-1) significantly reduced the proportion of g-MDSCs amongst the tumor-infiltrating CD45+ cells.

Blood was obtained from mice at day 18, and serum was analysed for the levels of various different cytokines by analysis using the MACSPlex cytokine 10 Kit for mouse (Miltenyi Biotec).

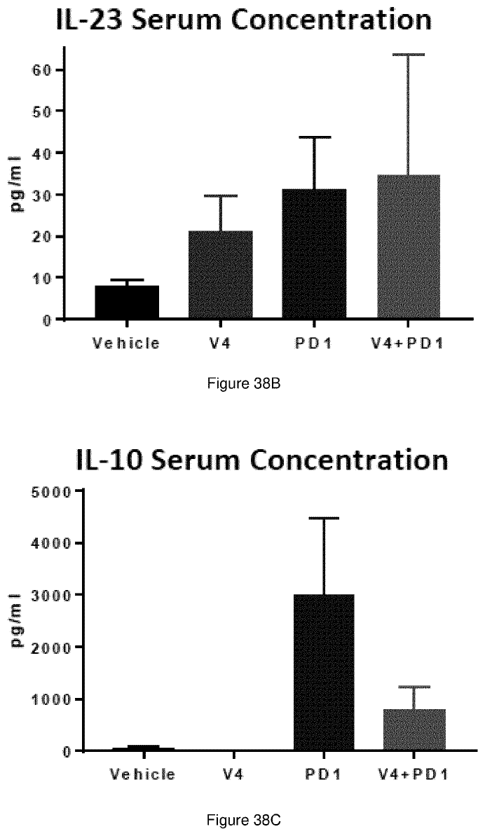

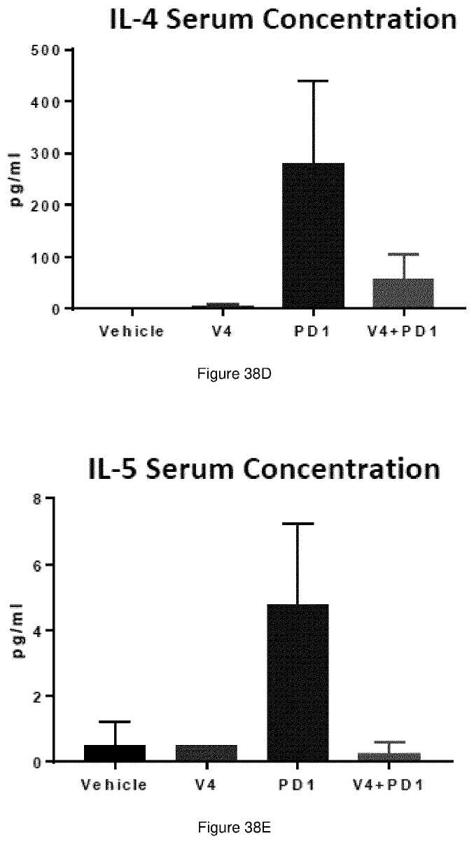

The results are shown in FIGS. 38A to 38E.

### 12.2 B16-BL6 Cell Model

FIGS. 40A and 40B show the results of the study described in Example 5.2.3 above (results shown in FIG. 17), extended to 18 days. Combination therapy with anti-VISTA antibody 4M2-C12 and anti-PD-1 inhibited tumor growth to a greater extent than either agent used alone.

Immunoprofiling of the tumor-infiltrating CD45+ cells was undertaken. Briefly, at day 18 of the experiment tumors were harvested, processed into single cell suspensions, stained with antibodies and specific for immune cell surface, analysed by flow cytometry and cells were classified into immune cell subsets as described in Example 12.1 above.

The percentage of tumor-infiltrating CD45+ cells having the indicated phenotypes are summarised below:

| | CD4 | CD8 | | | |
|---|---|---|---|---|---|
| Treatment Group | cells | cells | Treg | g-MDSC | m-MDSC |
| PBS | 2.72% | 8.79% | 1.42% | 2.06% | 6.68% |
| 4M2-C12 IgG2a | 2.84% | 10.7% | 1.58% | 1.65% | 13.94% |
| anti-PD-1 antibody | 0.95% | 3.43% | 0.51% | 36.65% | 14.12% |
| 4M2-C12 IgG2a + | 3.58% | 10.6% | 0.82% | 5.45% | 14.44% |
| anti-PD-1 antibody | | | | | |

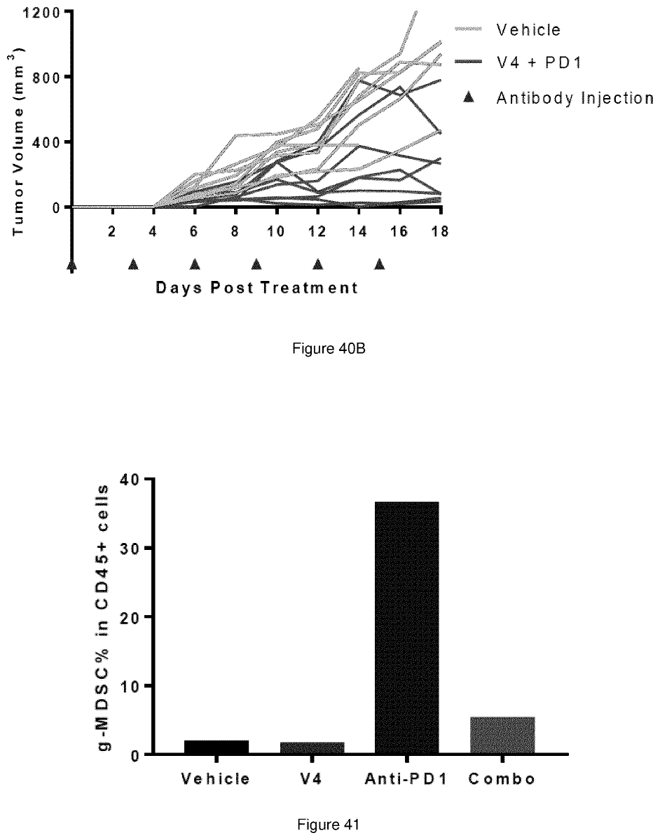

The percentage of tumor-infiltrating CD45+ cells which were g-MDSC is shown in FIG. 41.

### 12.3 EL4 Cell Model

A syngeneic model of T cell leukemia/lymphoma was established by injecting 2×10⁵ EL4 cells subcutaneously into the right flank of C57BL/6 mice.

Mice (7 per treatment group) were administered intraperitoneally every 3 days for a total of 5 doses with:
600 μg of 4M2-C12 IgG2a ([17] of Example 5)
200 μg of anti-PD-1 antibody (clone RMP1-14 (Bioxcell))
600 μg of 4M2-C12 IgG2a+200 μg of anti-PD-1 antibody
PBS only

Tumor volume was measured 3 times a week using a digital caliper and calculated using the formula [L×W2/2]. Study End point was considered to have been reached once the tumors of the control arm measured >1.5 cm in length.

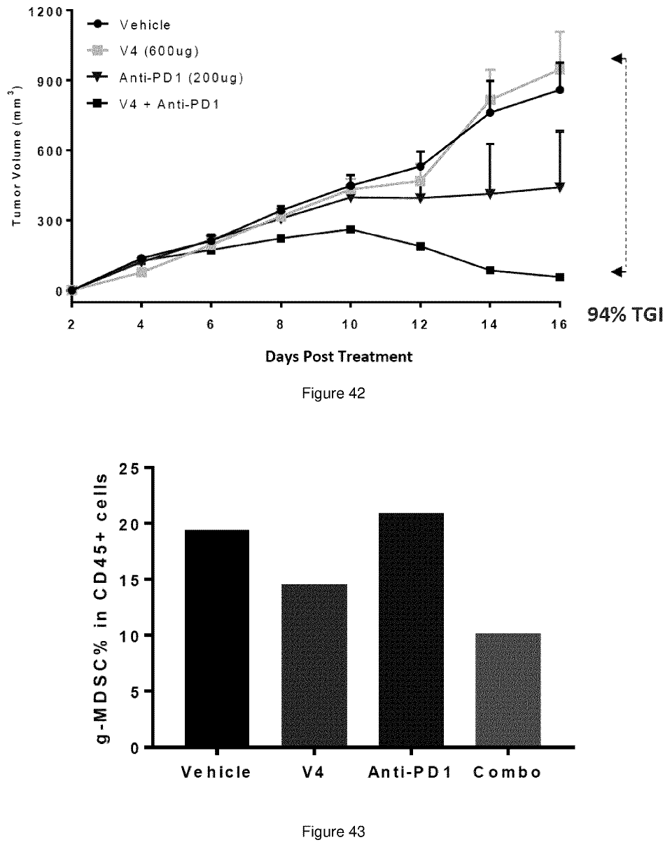

The results are shown in FIG. 42. Combination therapy with anti-VISTA antibody 4M2-C12 and anti-PD-1 inhibited tumor growth to a greater extent than either agent used alone.

Immunoprofiling of the tumor-infiltrating CD45+ cells was undertaken. Briefly, at day 16 of the experiment tumors were harvested, processed into single cell suspensions, stained with antibodies and specific for immune cell surface, analysed by flow cytometry and cells were classified into immune cell subsets as described in Example 12.1 above.

The percentage of tumor-infiltrating CD45+ cells having the indicated phenotypes are summarised below:

| | CD4 | CD8 | | | |
|---|---|---|---|---|---|
| Treatment Group | cells | cells | Treg | g-MDSC | m-MDSC |
| PBS | 3.71% | 0.55% | 0.18% | 19.41% | 0.88% |
| 4M2-C12 IgG2a | 7.4% | 1.84% | 0.19% | 14.51% | 0.94% |
| anti-PD-1 antibody | 4.35% | 3.04% | 0.09% | 20.91% | 0.81% |
| 4M2-C12 IgG2a + | 6.53% | 2.18% | 0.35% | 10.16% | 0.33% |
| anti-PD-1 antibody | | | | | |

The percentage of tumor-infiltrating CD45+ cells which were g-MDSC is shown in FIG. 43. Treatment with 4M2-C12 (either alone, or in combination with anti-PD-1) significantly reduced the proportion of g-MDSCs amongst the tumor-infiltrating CD45+ cells.

### 12.4 Conclusions

Inhibition of PD-1/PD-L1 signalling increases the proportion of g-MDSCs amongst the tumor-infiltrating CD45+ cells in the CT26, B16-BL6 and EL4 models, whereas treatment with 4M2-C12 suppresses g-MDSC expansion.

### Example 13: Further Characterisation of VISTA-Binding Antibodies

Further VISTA-binding antigen-binding molecules were produced:

| Antigen- | | |
|---|---|---|
| biding | | |
| molecule | Polypeptides | Antibody |
| [21] | V4-C1 VH-CH1-CH2-CH3 (SEQ ID NO: 311) | anti-VISTA clone V4-C1 IgG1 |
| | + | |
| | V4-C1 VL-CK (SEQ ID NO: 312) | |
| [22] | V4-C9 VH-CH1-CH2-CH3 (SEQ ID NO: 313) | anti-VISTA clone V4-C9 IgG1 |
| | + | |
| | V4-C9 VL-CK (SEQ ID NO: 314) | |
| [23] | V4-C24/C26/C27/C28/C30/C31 VH-CH1- | anti-VISTA clone V4-C24 IgG1 |
| | CH2-CH3 (SEQ ID NO: 315) | |
| | + | |
| | V4-C24 VL-CK (SEQ ID NO: 316) | |
| [24] | V4-C24/C26/C27/C28/C30/C31 VH-CH1- | anti-VISTA clone V4-C26 IgG1 |
| | CH2-CH3 (SEQ ID NO: 315) | |
| | + | |
| | V4-C26 VL-CK (SEQ ID NO: 317) | |
| [25] | V4-C24/C26/C27/C28/C30/C31 VH-CH1- | anti-VISTA clone V4-C27 IgG1 |
| | CH2-CH3 (SEQ ID NO: 315) | |
| | + | |
| | V4-C27 VL-CK (SEQ ID NO: 318) | |
| [26] | V4-C24/C26/C27/C28/C30/C31 VH-CH1- | anti-VISTA clone V4-C28 IgG1 |
| | CH2-CH3 (SEQ ID NO: 315) | |
| | + | |
| | V4-C28 VL-CK (SEQ ID NO: 319) | |
| [27] | V4-C24/C26/C27/C28/C30/C31 VH-CH1- | anti-VISTA clone V4-C30 IgG1 |
| | CH2-CH3 (SEQ ID NO: 315) | |
| | + | |
| | V4-C30 VL-CK (SEQ ID NO: 320) | |
| [28] | V4-C24/C26/C27/C28/C30/C31 VH-CH1- | anti-VISTA clone V4-C31 IgG1 |
| | CH2-CH3 (SEQ ID NO: 315) | |
| | + | |
| | V4-C31 VL-CK (SEQ ID NO: 321) | |

5 V4-C1, V4-C9, V4-C24, V4-C26, V4-C27, V4-C28, V4-C30 and V4-C31 were analysed in silico for safety and immunogenicity using IMGT DomainGapAlign (Ehrenmann et al., Nucleic Acids Res., 38, D301-307 (2010)) and IEDB deimmunization (Dhanda et al., Immunology. (2018) 153(1): 118-132) tools.

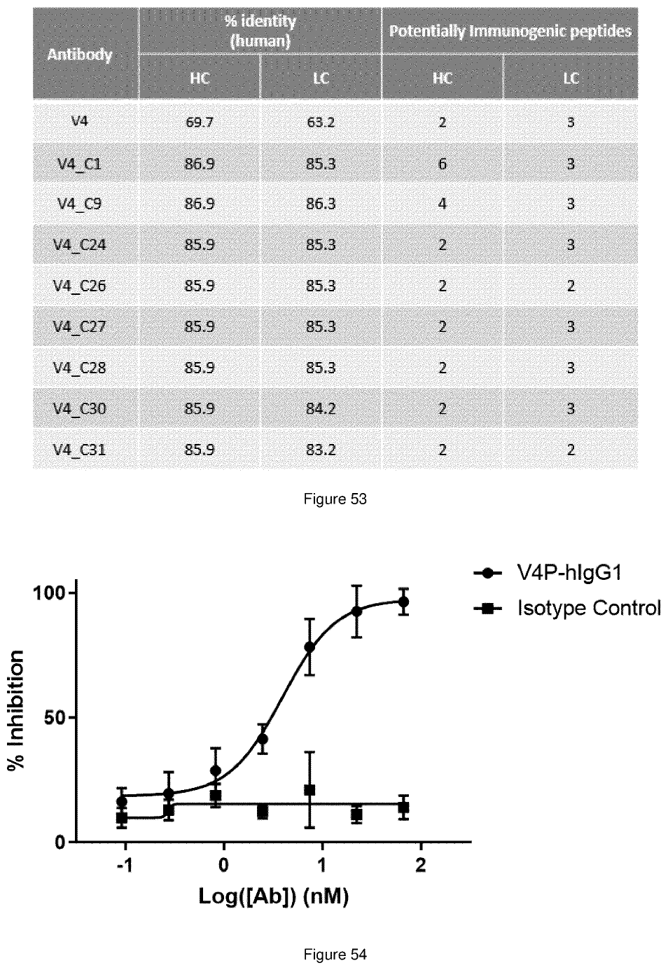

V4-C1, V4-C9, V4-C24, V4-C26, V4-C27, V4-C28, V4-C30 and V4-C31 had sufficient homology to human heavy and light chains to be considered humanized (i.e. >85%), had numbers of potentially immunogenic peptides few enough to be considered safe (see FIG. 53), and did not possess any other properties that could cause potential developability issues.

### 13.1 Analysis of Binding Affinity by BLI

Binding of V4-C1, V4-C9, V4-C24, V4-C26, V4-C27, V4-C28, V4-C30 and V4-C31 (i.e. to [28]) to human and mouse VISTA proteins and human PD-L1 was assessed by BLI using a Pall ForteBio Octet QK 384 system.

Briefly, anti-Penta-HIS biosensors were incubated for 60 sec in PBS buffer (pH 7.2) to obtain the first baseline, and were subsequently loaded for 120 sec with 180 nM hVISTA, 180 nM mVISTA or 250 nM hPD-L1 in PBS (pH 7.2). After loading, biosensors were incubated for 60 sec in PBS buffer pH 7.2 to obtain the second baseline, and for 120 sec or 900 sec with a 6 point, 2 fold dilution series of the test antibodies (500 nM-15.6 nM) in PBS pH 7.2 to obtain the association curves. Finally, the biosensors were incubated for 120 sec in PBS pH 7.2 to obtain the dissociation curves. Kinetic and affinity constants were calculated by global fitting of the association and dissociation data to a 1:1 binding model.

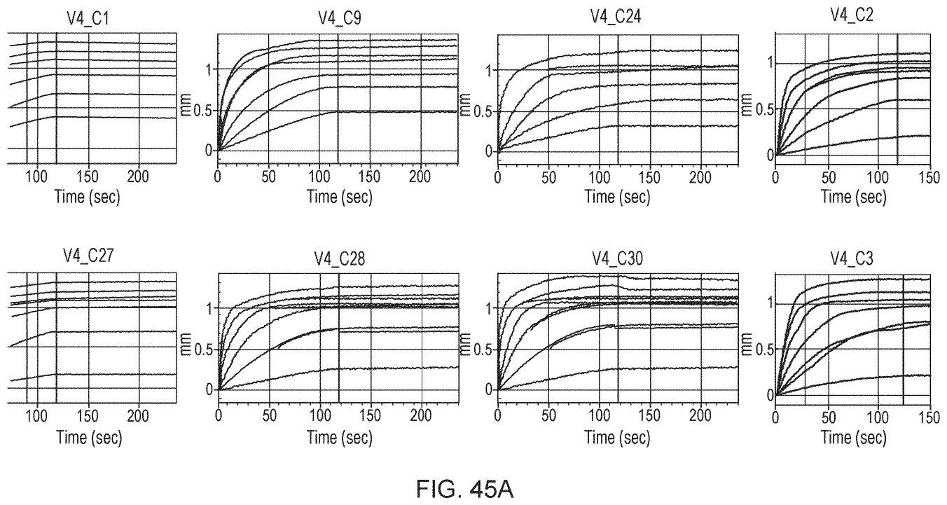

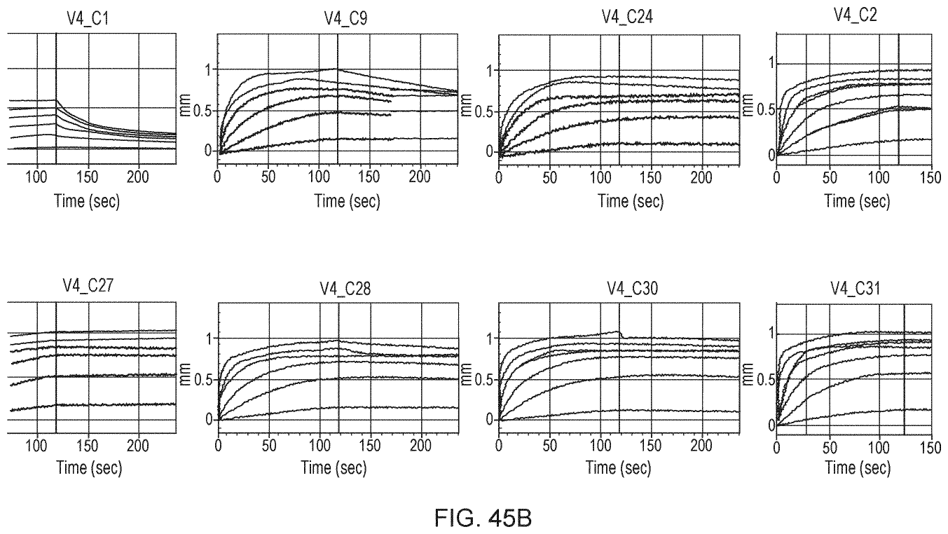

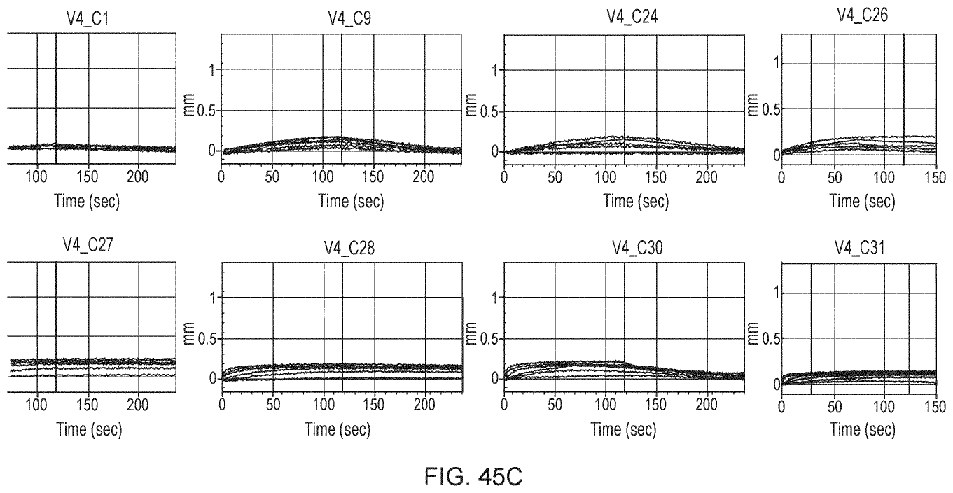

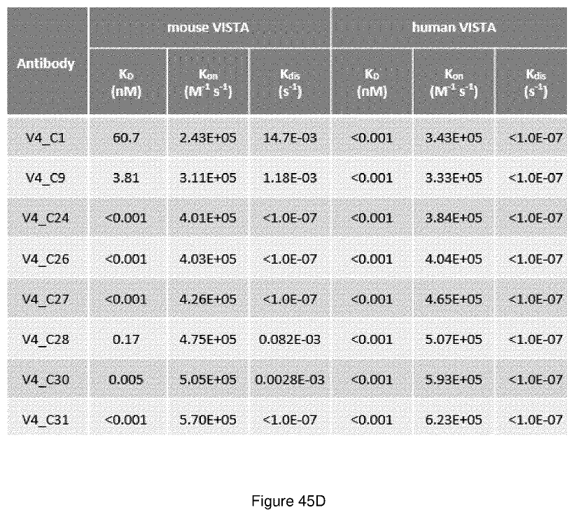

None of V4-C1, V4-C9, V4-C24, V4-C26, V4-C27, V4-C28, V4-C30 or V4-C31 displayed significant binding to human PD-L1 (FIG. 45C).

The kinetic and thermodynamic constants calculated for binding of V4-C1, V4-C9, V4-C24, V4-C26, V4-C27, V4-C28, V4-C30 and V4-C31 to human VISTA and mouse VISTA in this experiment are shown in FIG. 45D.

Binding to mouse VISTA protein by V4-C1, V4-C9, V4-C24, V4-C26, V4-C27, V4-C28, V4-C30 and V4-C31 was analysed in a separate experiment, which included evaluation of VSTB112 IgG1 (comprising VSTB112 HC (SEQ ID NO: 269)+VSTB112 LC (SEQ ID NO: 270)).

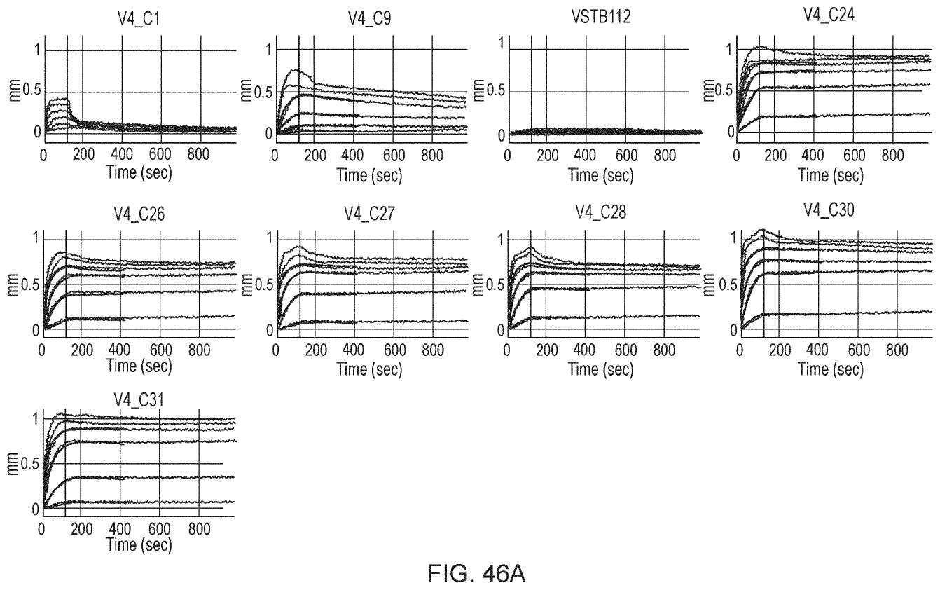

VSTB112 did not display significant binding to mouse VISTA protein (FIG. 46A). The kinetic and thermodynamic constants calculated for binding of V4-C1, V4-C9, V4-C24, V4-C26, V4-C27, V4-C28, V4-C30 and V4-C31 to mouse VISTA in this experiment are shown in FIG. 46B.

In a further experiment, binding of V4-C1, V4-C9, V4-C24, V4-C26, V4-C27 and VSTB112 IgG1 to human VISTA and mouse VISTA was analysed, and the calculated kinetic and thermodynamic constants are shown in FIG. 47C.

In another experiment, binding of V4 ([1] of Example 2.2) and VSTB112 IgG1 (comprising VSTB112 HC (SEQ ID NO: 269)+VSTB112 LC (SEQ ID NO: 270)) to human VISTA, mouse VISTA and human CD47 was analysed. Anti-Penta-HIS biosensors were incubated for 60 sec in PBS buffer (pH 7.2) to obtain the first baseline, and were subsequently loaded for 120 sec with 180 nM hVISTA, 180 nM mVISTA or 300 nM hCD47 in PBS (pH 7.2). After loading, biosensors were incubated for 60 sec in PBS buffer pH 7.2 to obtain the second baseline, and for 120 sec with a dilution series of the test antibodies (1500 nM-46.9 nM) in PBS pH 7.2 to obtain the association curves. Finally, the biosensors were incubated for 120 sec in PBS pH 7.2 to obtain the dissociation curves. Kinetic and affinity constants were calculated by global fitting of the association and dissociation data to a 1:1 binding model.

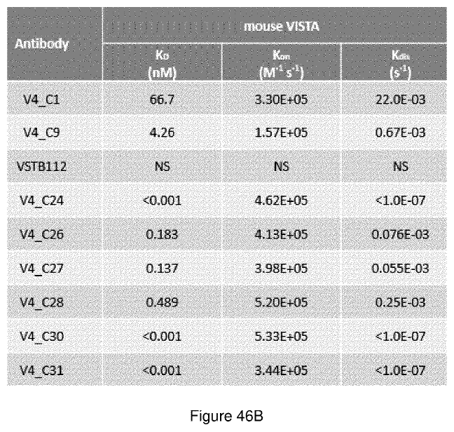

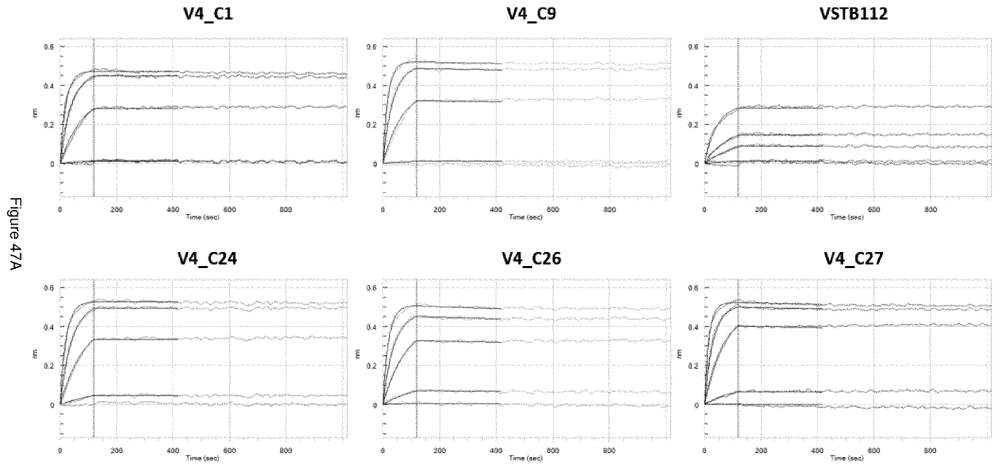

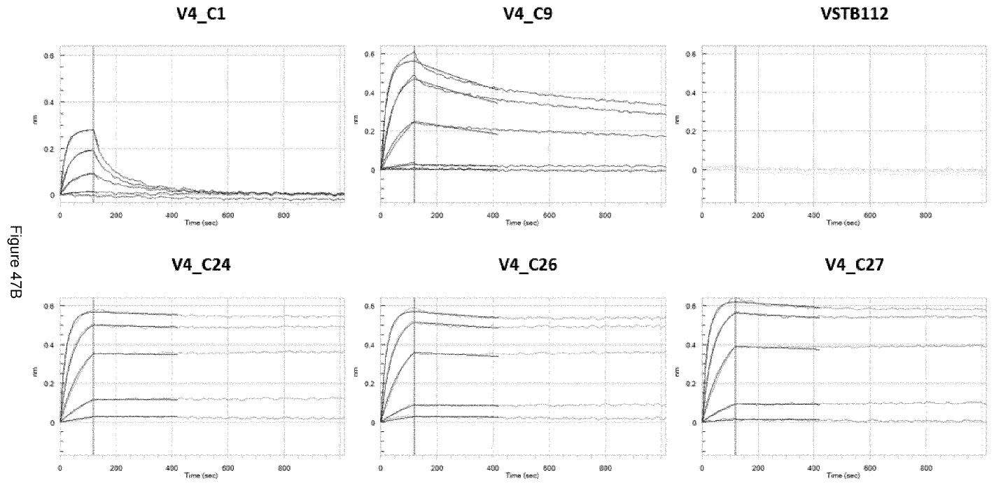

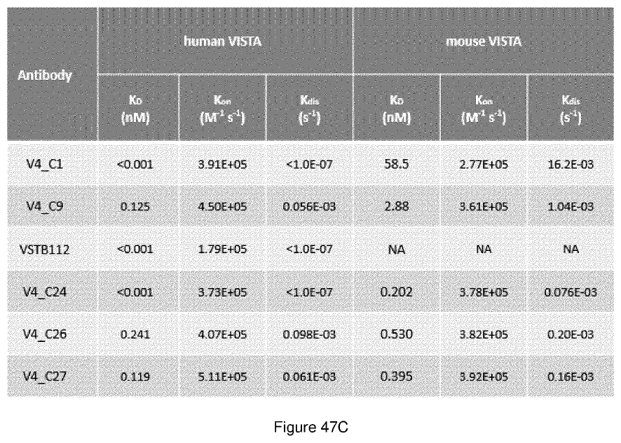

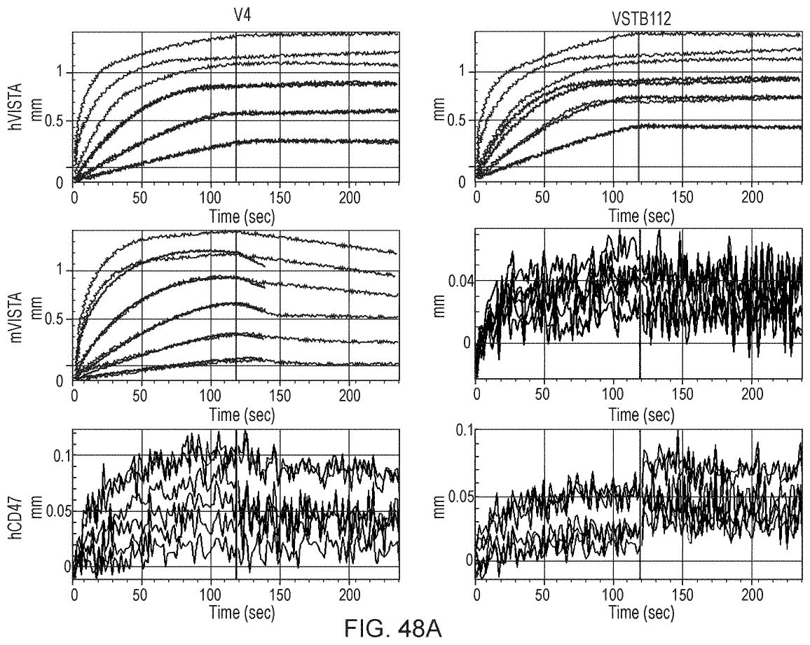

Neither V4 nor VSTB112 displayed binding to human CD47. VSTB112 did not display significant binding to mouse VISTA protein, whereas V4 did. The calculated kinetic and thermodynamic constants are shown in FIG. 48B.

### 13.2 Analysis of Binding Affinity by ELISA

ELISAs were used to evaluate binding of different antibodies to human VISTA and mouse VISTA. The ELISAs were performed as described in Example 3.3 above.

The following antibodies were analysed in the experiments:
4M2-C12 IgG1 ([1] of Example 2.2; referred to as “V4pr” in the Figures)
V4-C1 IgG1 ([21] of Example 13)
V4-C9 IgG1 ([22] of Example 13)
V4-C24 IgG1 ([23] of Example 13)
V4-C26 IgG1 ([24] of Example 13)
V4-C27 IgG1 ([25] of Example 13)
V4-C28 IgG1 ([26] of Example 13)
V4-C30 IgG1 ([27] of Example 13)
V4-C31 IgG1 ([28] of Example 13)
VSTB112 IgG1 (comprising VSTB112 HC (SEQ ID NO: 269)+VSTB112 LC (SEQ ID NO: 270))
Atezolizumab
Human IgG1 Isotype control

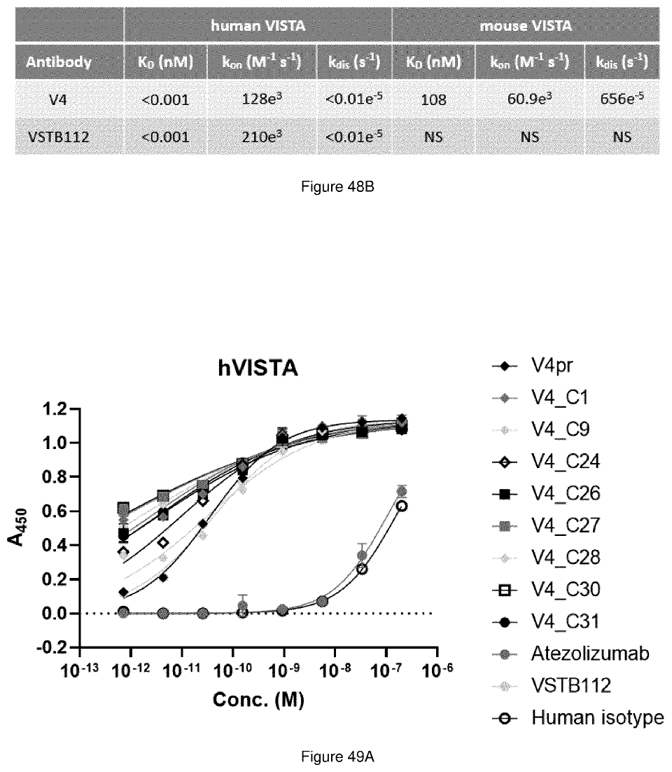

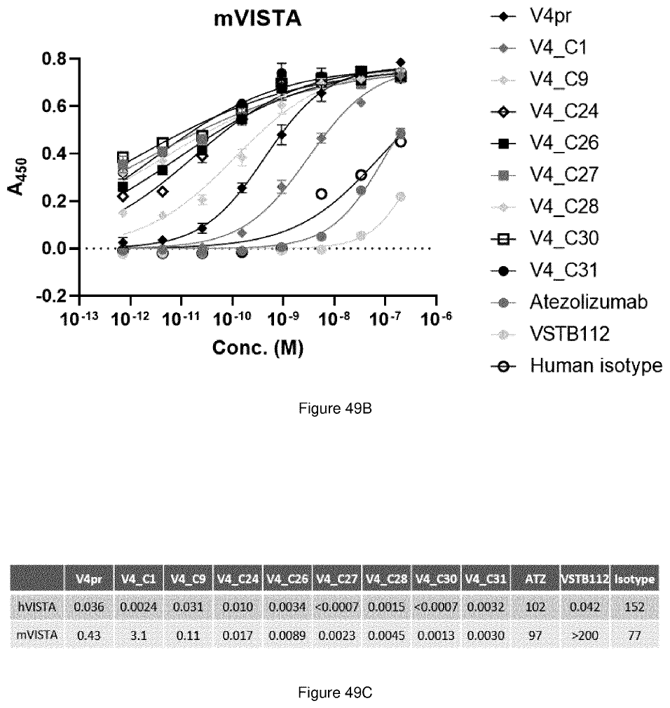

The results obtained are shown in FIGS. 49A and 49B, and the EC₅₀ (nM) values calculated from the ELISAs for binding of the antibodies to the indicated proteins are summarised in FIG. 49C.

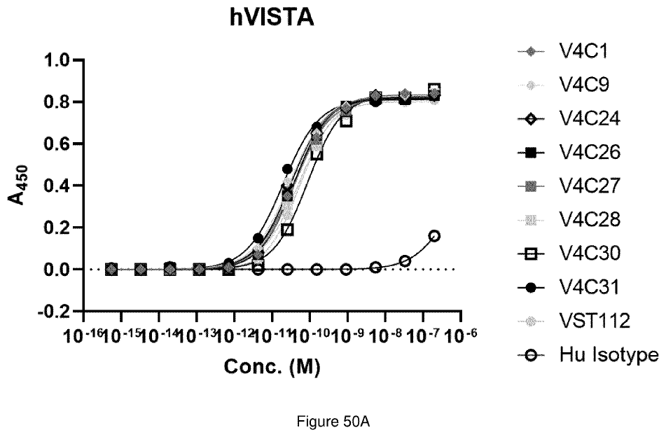

A further experiment was performed in which binding of V4-C1, V4-C9, V4-C24, V4-C26, V4-C27, V4-C28, V4-C30, V4-C31, VSTB112 and isotype control antibody to human VISTA or mouse VISTA was analysed. The results are shown in FIGS. 50A and 50B, and the EC₅₀ (nM) values calculated from the ELISAs for binding of the antibodies to the indicated proteins are summarised in FIG. 50C.

A further experiment was performed in which binding of V4-C1, V4-C9, V4-C24, V4-C26, V4-C27, VSTB112 and isotype control antibody to human VISTA or mouse VISTA was analysed. The results are shown in FIGS. 51A and 51B, and the EC₅₀ (nM) values calculated from the ELISAs for binding of the antibodies to the indicated proteins are summarised in FIG. 51C.

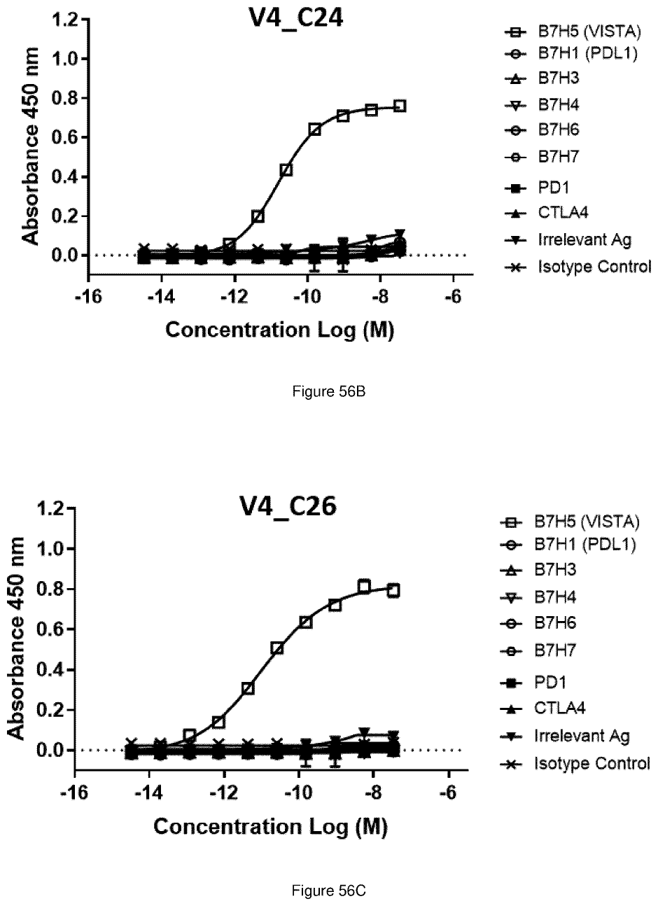

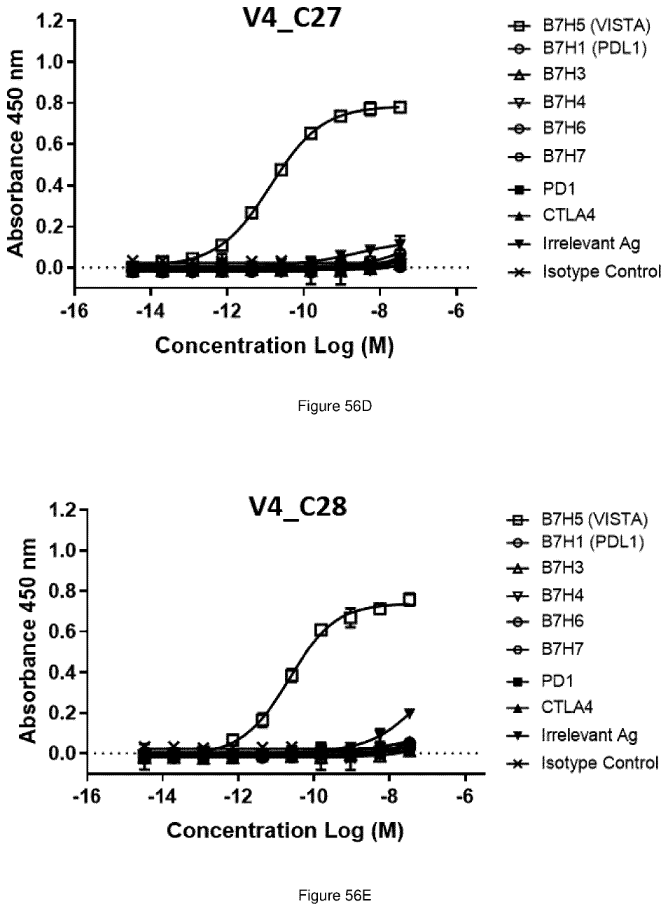

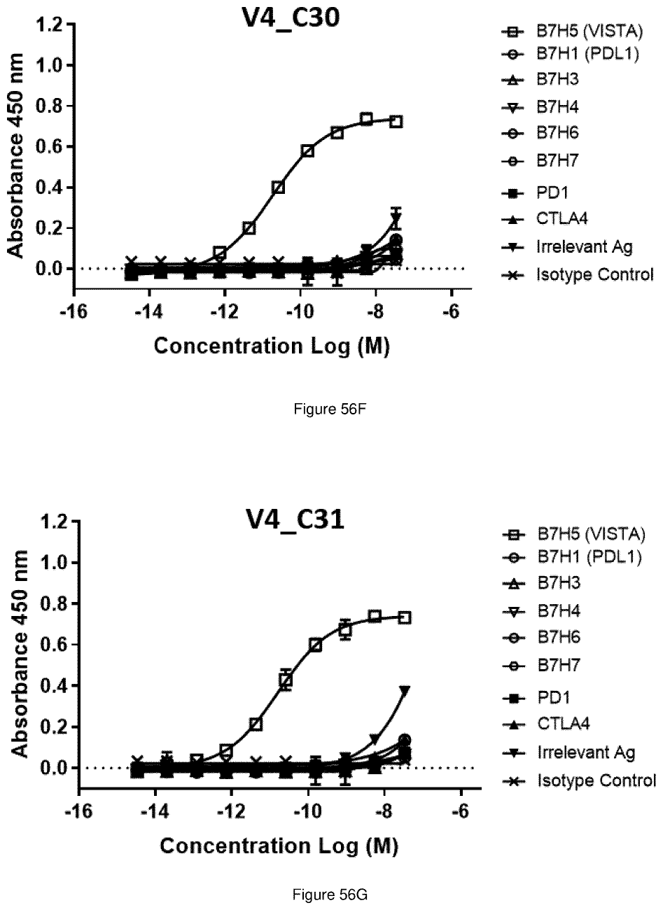

A further experiment was performed in which binding of V4, V4-C24, V4-C26, V4-C27, V4-C28, V4-C30 and V4-C31 and isotype control antibody to human VISTA, PD-L1, B7H3, B7H4, B7H6, B7H7, PD-1 and CTLA-4 was analysed. The results are shown in FIGS. 56A to 56G. Each of V4-C24, V4-C26, V4-C27, V4-C28, V4-C30 and V4-C31 displayed strong binding to human VISTA, and no cross-reactivity for other members of the B7 family of proteins.

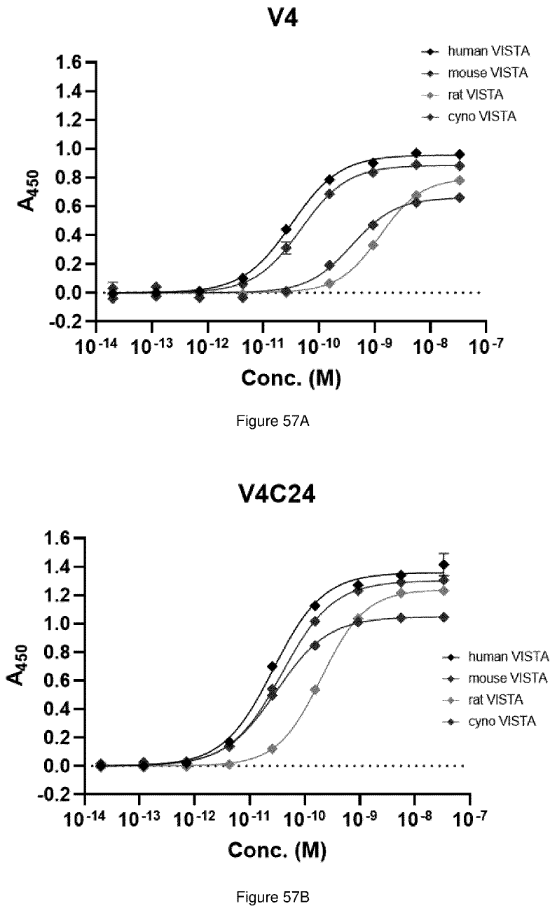

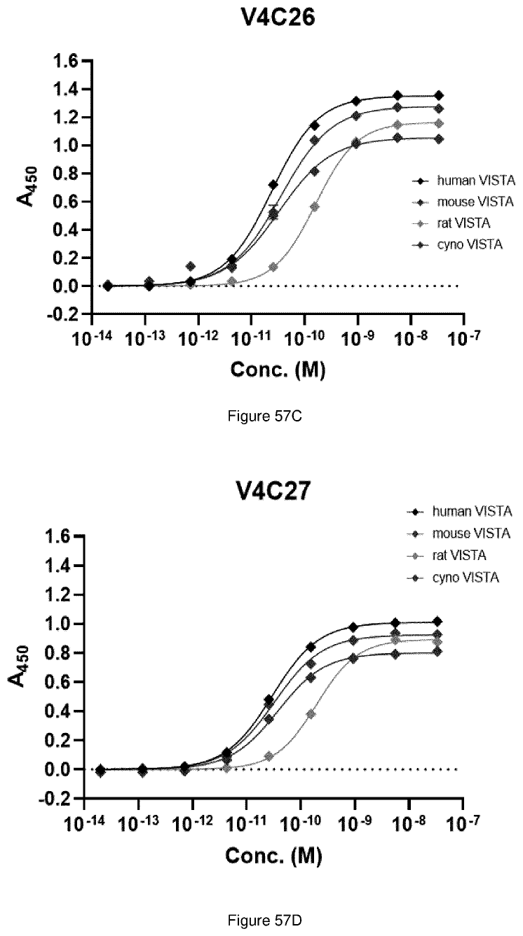

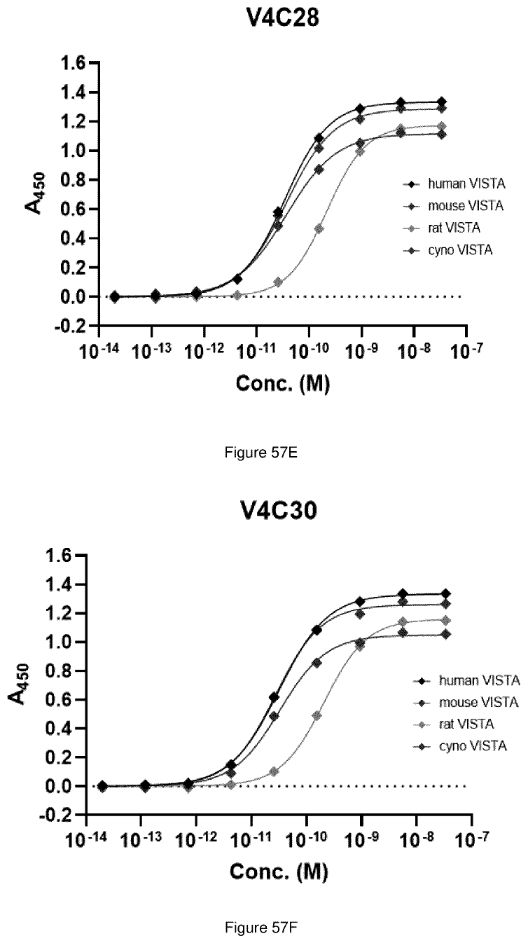

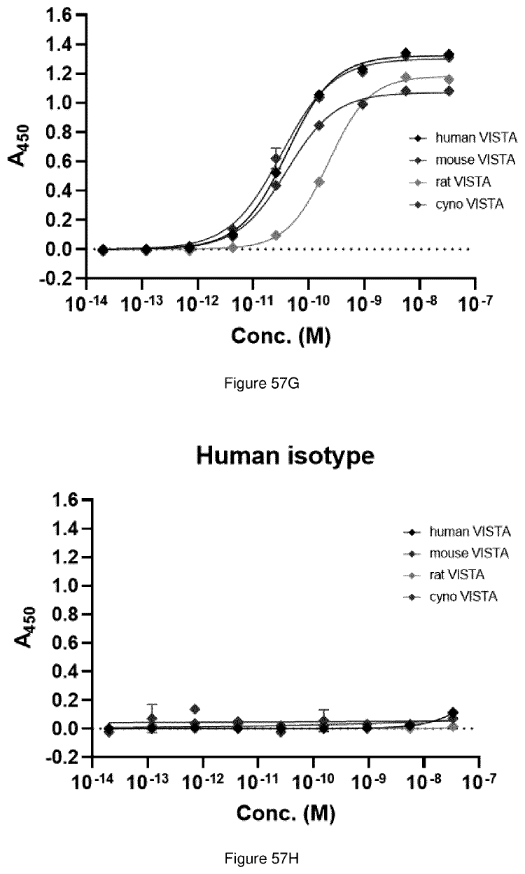

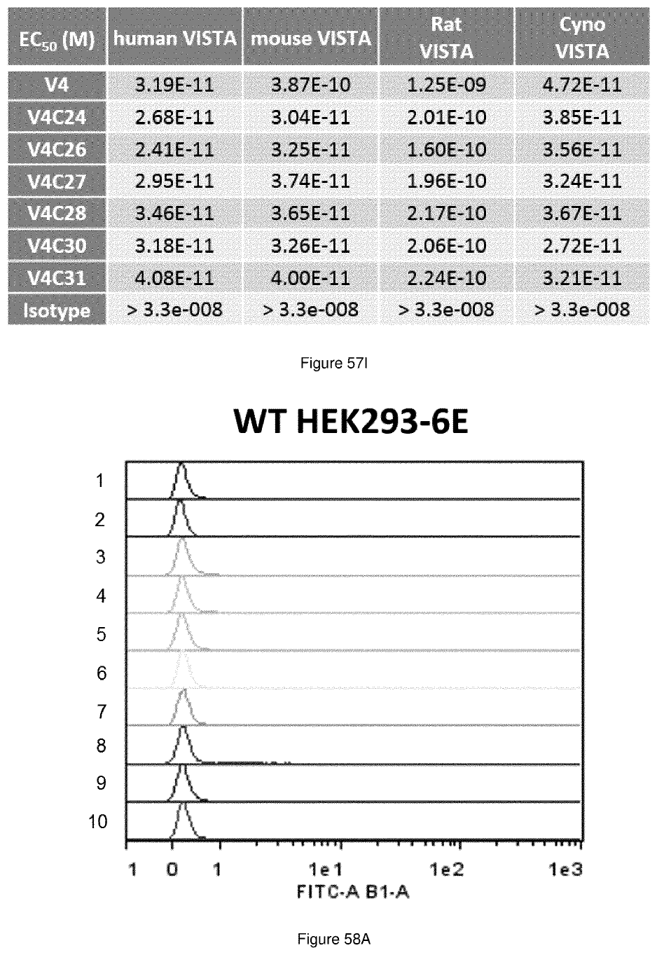

In a further experiment, V4 (referred to in FIG. 57 as “V4P”), V4-C24, V4-C26, V4-C27, V4-C28, V4-C30 and V4-C31 were analysed for binding to human VISTA, mouse VISTA, rat VISTA and cyno VISTA. The results obtained are shown in FIGS. 57A to 57H, and the EC₅₀ (M) values calculated from the ELISAs for binding of the antibodies to the indicated proteins are summarised in FIG. 57I.

V4 and all of the V4-derived clones V4-C24, V4-C26, V4-C27, V4-C28, V4-C30 and V4-C31 were found to bind to human VISTA, mouse VISTA, rat VISTA and cyno VISTA.

### 13.3 Analysis of Binding to VISTA-Expressing Cells by Flow Cytometry

Anti-VISTA antibodies were analysed for their ability to bind to VISTA-expressing cells essentially as described in Example 3.1 above.

Briefly, transfected cells, or HEK293 cells transfected with vector encoding human VISTA or mouse VISTA were incubated with 1 μg/ml of anti-VISTA antibody or isotype control antibody at 4° C. for 1 hr. Cells were then washed, and incubated with 10 μg/ml FITC-conjugated anti-human Fc antibody at 4° C. for 1 hr. Cells were washed again, and then analysed by flow cytometry.

The following antibodies were analysed in the experiments:
4M2-C12 IgG1 ([1] of Example 2.2; referred to as “V4P” in the Figures)
V4-C24 IgG1 ([23] of Example 13)
V4-C26 IgG1 ([24] of Example 13)
V4-C27 IgG1 ([25] of Example 13)
V4-C28 IgG1 ([26] of Example 13)
V4-C30 IgG1 ([27] of Example 13)
V4-C31 IgG1 ([28] of Example 13)
VSTB112 IgG1 (comprising VSTB112 HC (SEQ ID NO: 269)+VSTB112 LC (SEQ ID NO: 270))
Human IgG1 Isotype control

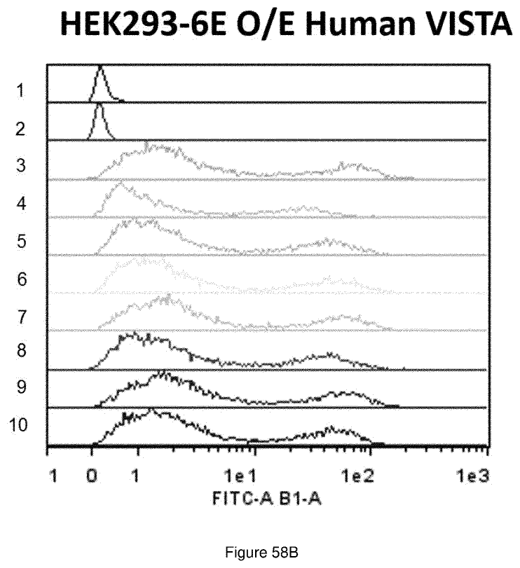

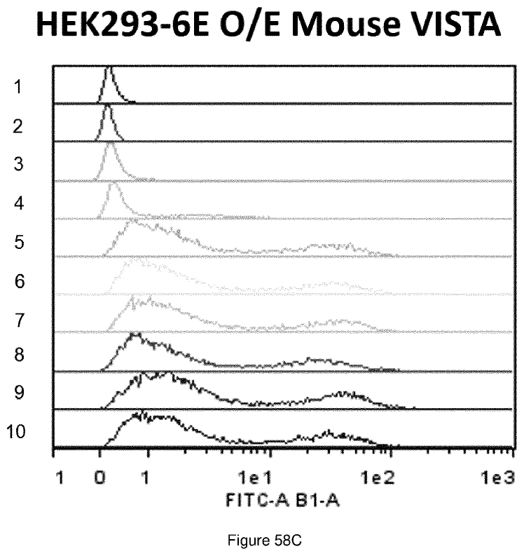

The results are shown in FIGS. 58A to 58C.

### 13.4 Analysis of Thermostability by Differential Scanning Fluorimetry

Thermostability of different antibodies was evaluated by Differential Scanning Fluorimetry analysis, as described in Example 3.4 above.

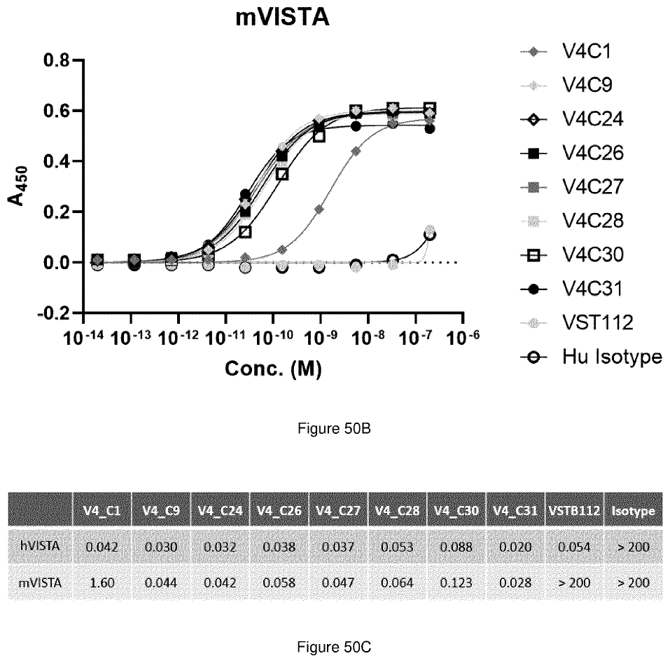

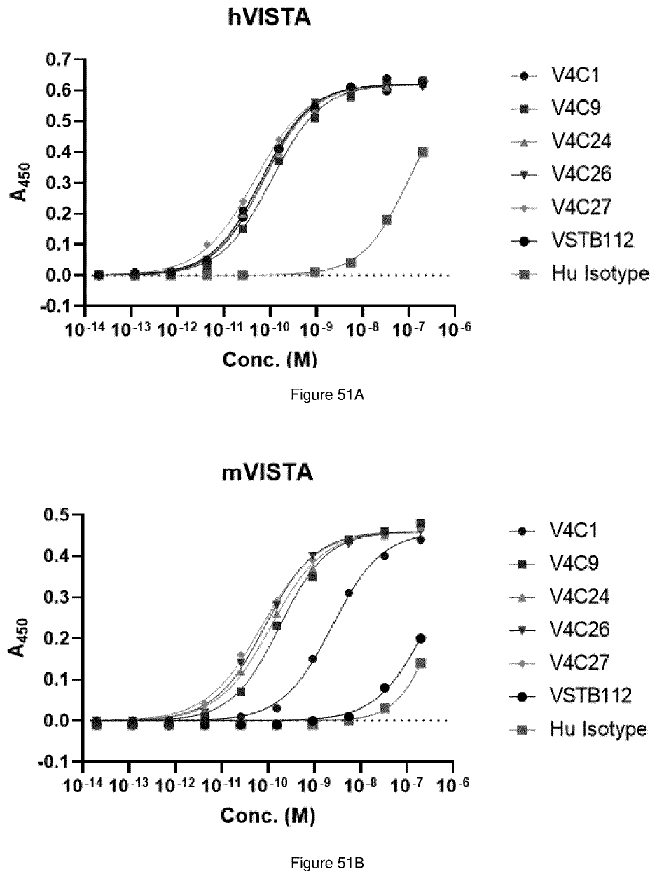

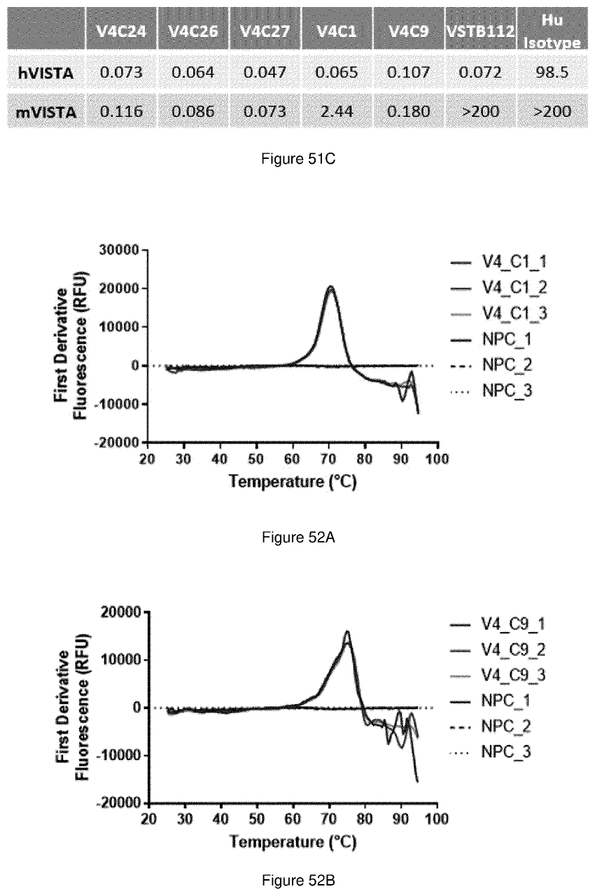

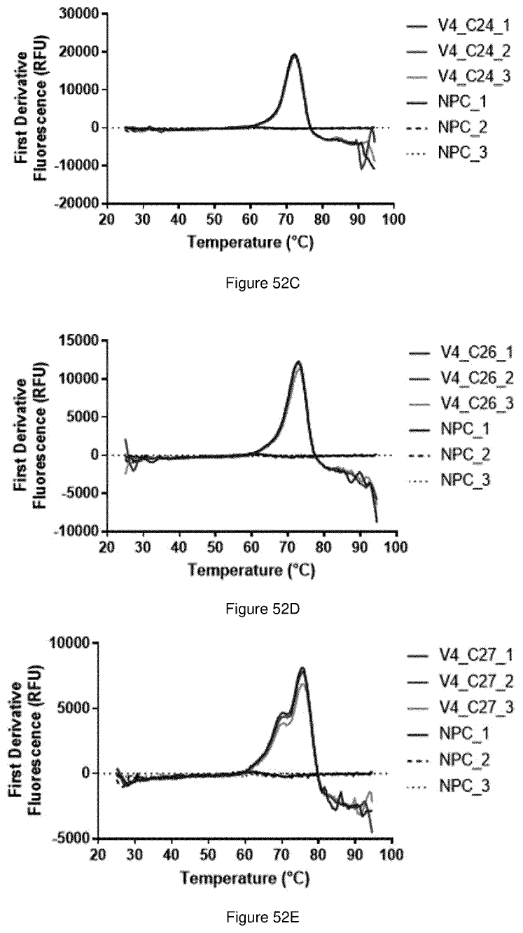

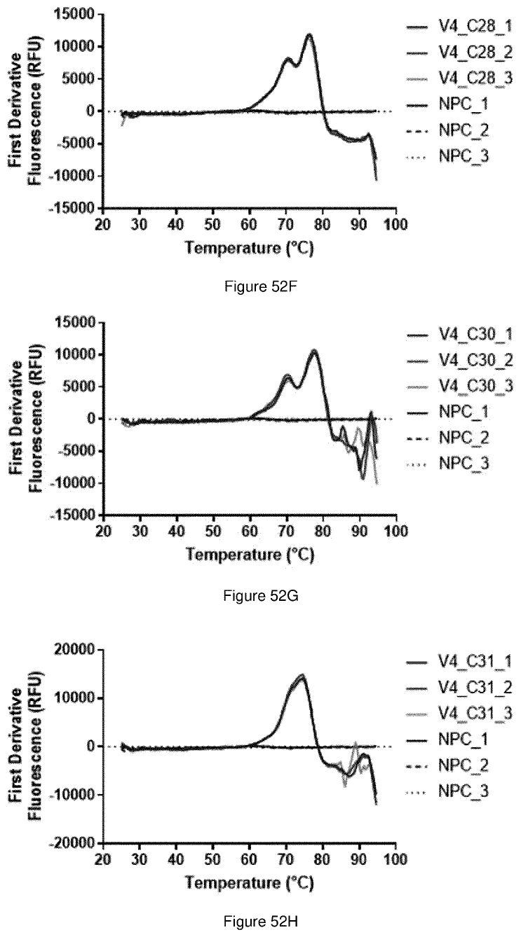

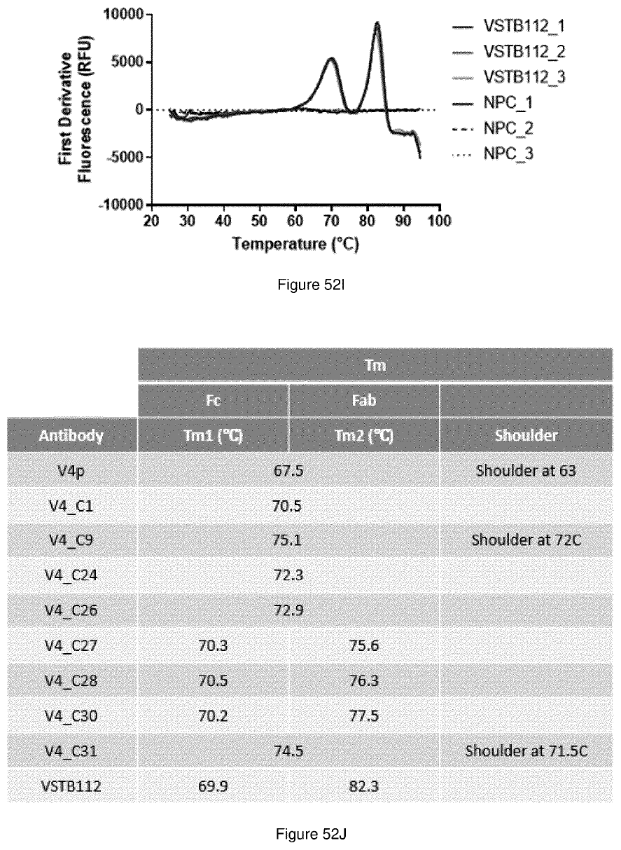

The first derivative of the raw data obtained for Differential Scanning Fluorimetry analysis of the thermostability of V4-C1, V4-C9, V4-C24, V4-C26, V4-C27, V4-C28, V4-C30, V4-C31 (i.e. to [28]) and VSTB112 (in triplicate) is shown in FIGS. 52A to 52I, and the results are summarised in FIG. 52J.

V4-C1, V4-C9, V4-C24, V4-C26, V4-C27, V4-C28, V4-C30 and V4-C31 were found to have a higher melting temperature (Tm) for the Fab region as compared to V4 (67.5° C.), and thus improved thermal stability.

### Example 14: Use of VISTA-Binding Antibodies in Immunohistochemistry

Anti-VISTA antibody 4M2-C12 mIgG2a ([17] of Example 5) was evaluated for its ability to be used in immunohistochemistry for the detection of human VISTA protein.

Processing of sections was performed using Bond reagents (Leica Biosystems). Commercial paraffin sections from normal human spleen or normal human ovary were de-paraffinized in Bond Dewax solution, and rehydrated using. Sections were then subjected to the following treatments with 4-5 rinses of 1× Bond Wash between steps: (i) antigen exposure by treatment with Bond Epitope Retrieval Solution for 40 min at 100° C., (ii) endogenous peroxidase blocking by treatment with 3.5% (v/V) H₂O₂ for 15 min at room temperature, (iii) blocking by treatment with 10% goat serum for 30 min at room temperature, (iv) incubation with 4M2-C12 mIgG2a at 1:50 dilution of a 9.37 mg/mL solution overnight at 4° C., (v) incubation with HRP-polymer conjugated goat anti-mouse antibody for 5 min at room temperature, and (vi) development with Bond Mixed DAB Refine for 7 min at room temperature, followed by rinsing with deionised water to stop the reaction.

Sections were counterstained with haematoxylin for 5 min at room temperature and rinsed with deionised water and 1× Bond Wash solution, and were then dehydrated, mounted in synthetic mounting media and scanned with high resolution.

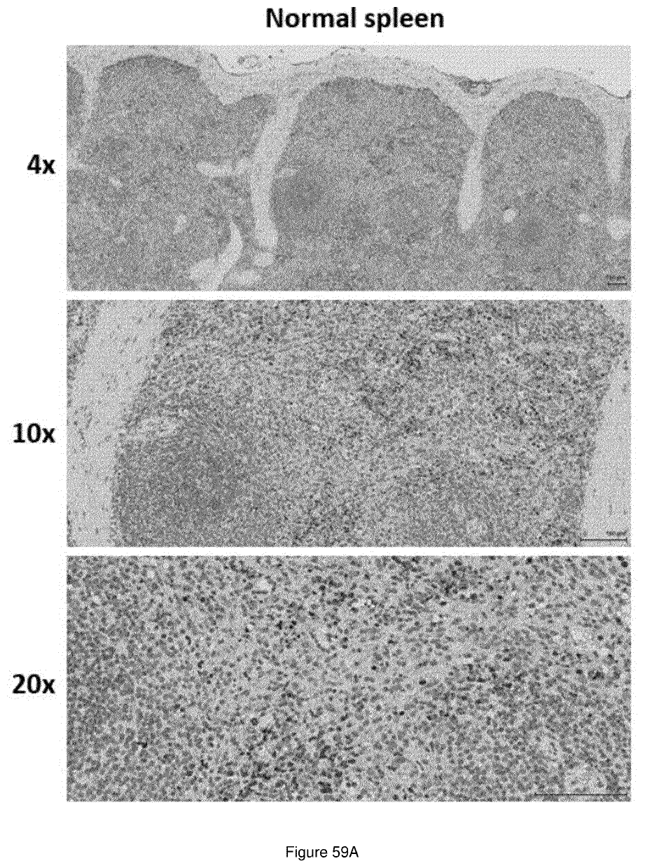

The results are shown in FIGS. 59A and 59B. The anti-VISTA antibody stained cytoplasm of cells of the spleen, but not cells in normal ovary sections (control).

### Example 15: Further Analysis of the Ability of VISTA-Binding Antibodies to Rescue VISTA-Mediated Inhibition of T Cell Proliferation and Production of Proinflammatory Cytokines

Anti-VISTA antibodies were characterised for the ability to release T cells from VISTA-mediated suppression.

96-well plates were coated with anti-CD3 at concentration of 2.5 μg/ml and incubated overnight at 4° C. PBMCs were isolated from blood samples, T cells were enriched from the PBMCs and labelled with CSFE as above, and the CFSE-labelled T cell were then co-cultured at a ratio of 2:1 with HEK293-6E cells transfected with a construct encoding human VISTA, in RPMI 1640 medium supplemented with 2% FBS.

Cells were then treated with 4M2-C12-hIgG1 ([1] of Example 2.2) or VSTB112 at concentrations of 0 μg/ml (control), 20 μg/ml or 50 μg/ml.

After 5 days, cells were harvested and analysed by flow cytometry to determine cell proliferation by CSFE dilution profile. Cell culture supernatants were also harvested, and INFγ and TNFa levels was analysed by ELISA.

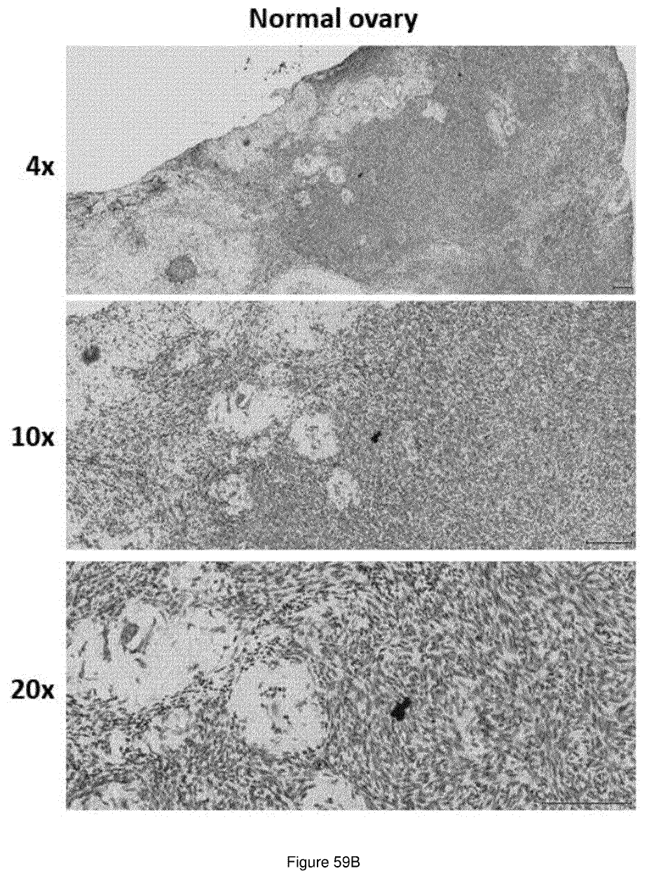

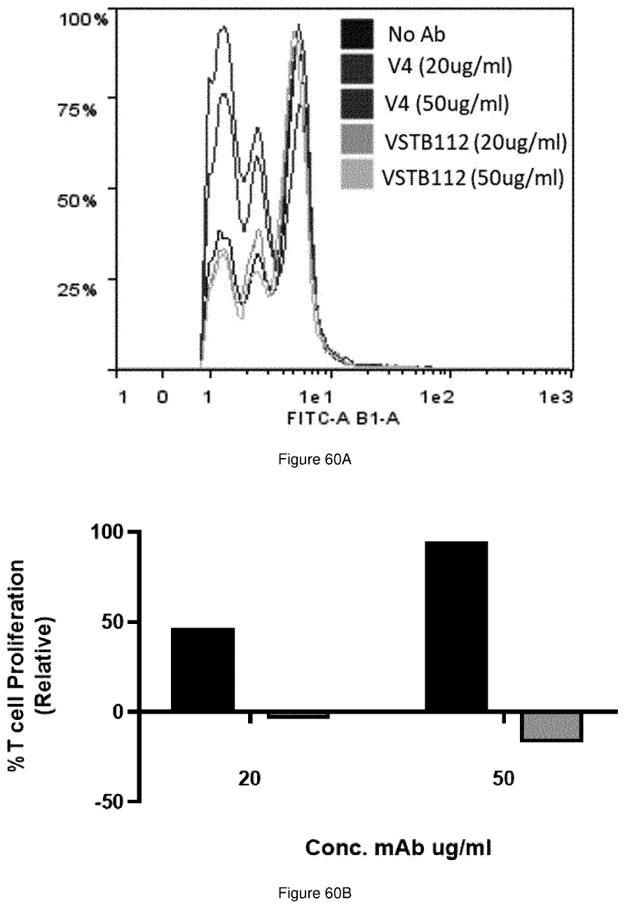

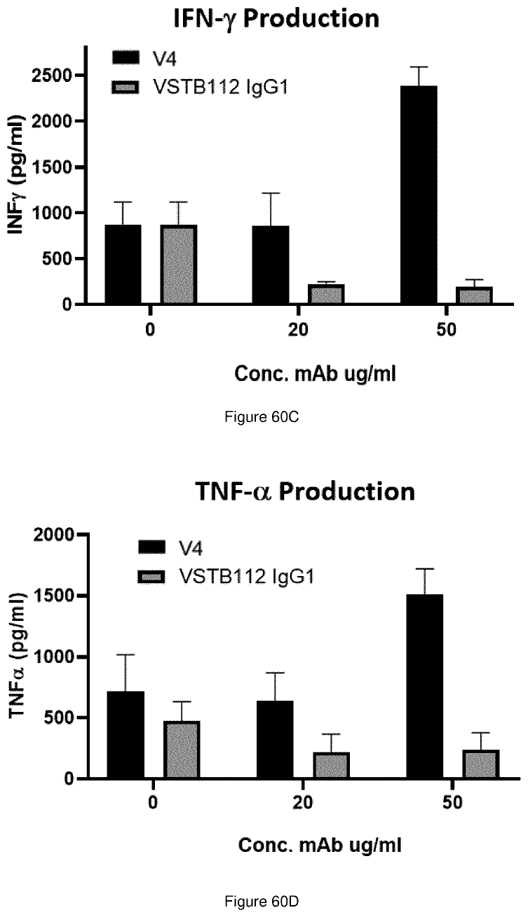

The results are shown in FIGS. 60A to 60D. FIGS. 60A and 60B show that 4M2-C12-hIgG1 released T cells from VISTA-mediated inhibition of proliferation in a dose-dependent fashion. FIGS. 60C and 60D show that 4M2-C12-hIgG1 released T cells from VISTA-mediated inhibition of production of INFγ and TNFa.

In further experiments, undifferentiated THP1 cells were seeded in wells of 96 well plates in duplicate, in RPMI media without FBS or pen/strep (100,000 cells/well), and cells were stimulated with LPS (100 μg/ml) in the presence of serially diluted concentrations of 4M2-C12-hIgG1 ([1] of Example 2.2) or VSTB112, at concentrations ranging from 2000 μg/ml to 7.8 μg/ml.

After 24 h cell culture supernatant was collected and analyzed by ELISA for IL-6 and TNFa. Cells were also fixed and permeabilized, and analyzed for the presence of VISTA via flow cytometry.

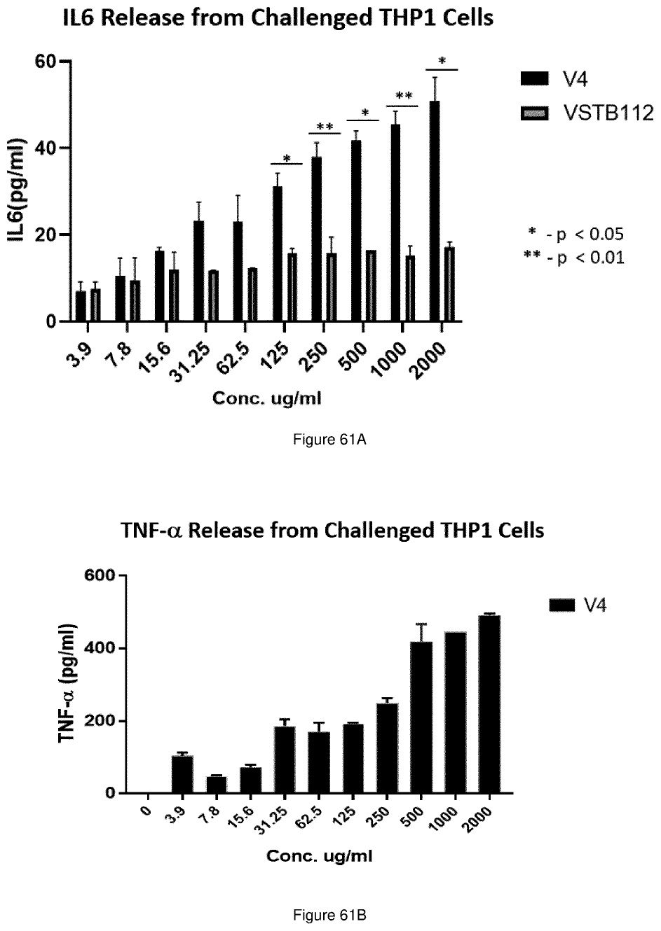

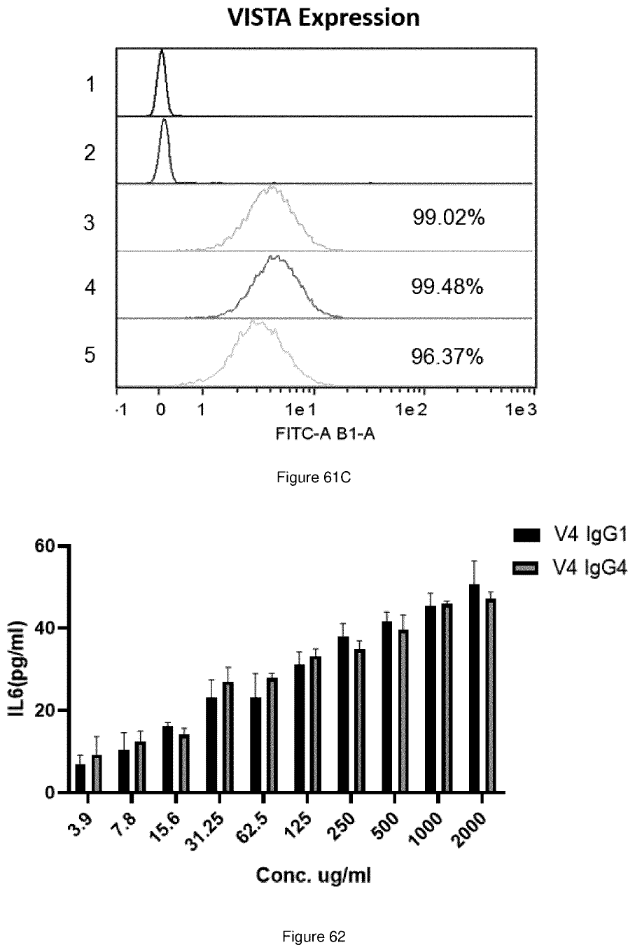

The results are shown in FIGS. 61A to 61C. 4M2-C12-hIgG1 was found to increase IL-6 and TNFa production from LPS-stimulated THP1 cells in a dose-dependent fashion, and to a much greater extent than VSTB112.

In a further experiment, undifferentiated THP1 cells were seeded in wells of 96 well plates in duplicate, in RPMI media without FBS or pen/strep (100,000 cells/well), and cells were stimulated with LPS (100 μg/ml) and MnCl₂ (100 μM) in the presence of 4M2-C12-hIgG1 ([1] of Example 2.2) or 4M2-C12-hIgG4 ([29] shown below), at concentrations ranging from 2000 μg/ml to 7.8 μg/ml. After 24 h cell culture supernatant was collected and analyzed by ELISA for IL-6.

| Antigen- | | |
|---|---|---|
| biding | | |
| molecule | Polypeptides | Antibody |
| [29] | 4M2-C12 VH-CH1-CH2-CH3 lgG4 (SEQ ID | anti-VISTA clone 4M2-C12 IgG4 |
| | NO: 330) | |
| | + | |
| | 4M2-C12 VL-CK (SEQ ID NO: 213) | |

The results are shown in FIG. 62. Increased production of IL-6 by LPS-stimulated THP1 cells was found to be independent of Fc-independent, as no significant difference was observed between the level of IL6 induced by treatment with 4M2-C12 in human IgG1 or IgG4 formats.

### Example 16: Further Analysis of Pharmacology, Toxicology and Immunotoxicity

In an acute dose study, rats were administered with a single dose of 10 mg/kg, 25 mg/kg, 100 mg/kg or 250 mg/kg of 4M2-C12-hIgG1 ([1] of Example 2.2) or 4M2-C12-hIgG4 ([29] of Example 15).

Blood was obtained from the rats at baseline (−2 hr), 0.5 hr, 6 hr, 24 hr, 96 hr, 168 hr and 336 hr after administration. Antibody in the serum was quantified be ELISA.

The parameters for the pharmacokinetic analysis were derived from a non-compartmental model: maximum concentration (Cₘₐₓ), AUC (0-336 hr), AUC (0-infinity), Half-life (t_{1/2}), Clearance (CL), Volume of distribution at steady state (Vₛₛ).

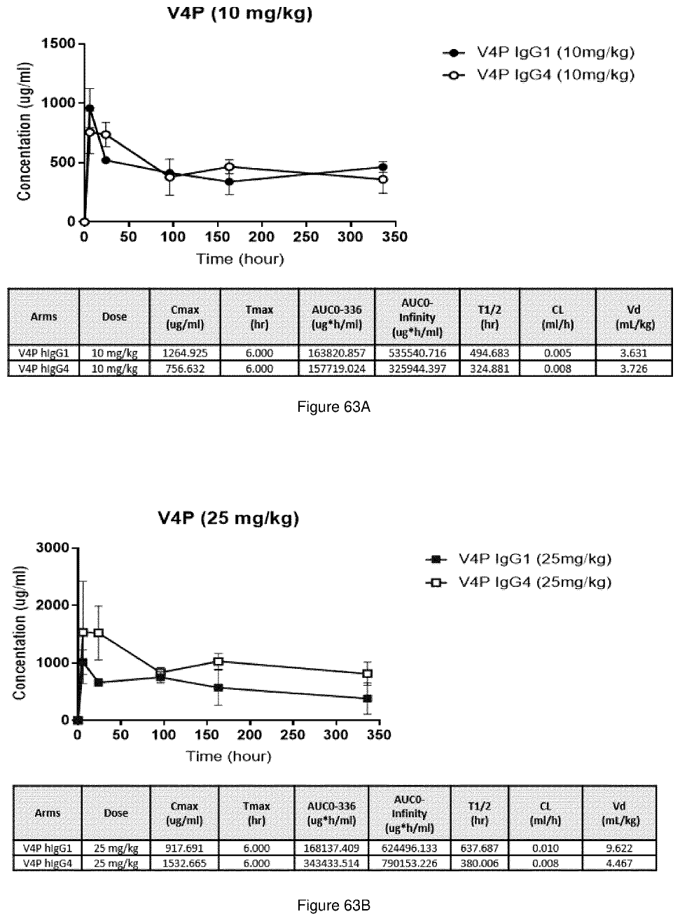

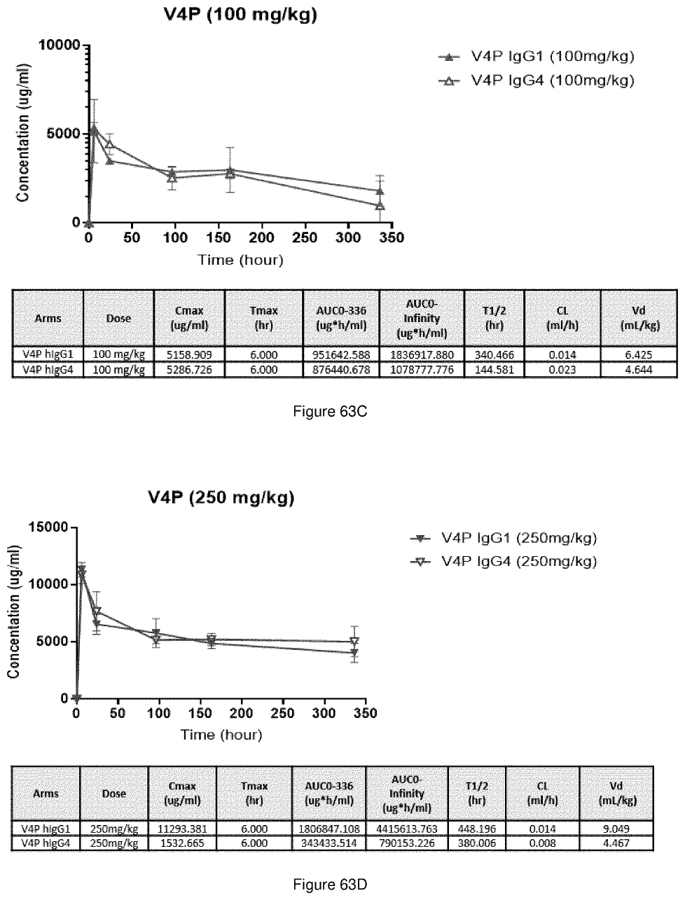

The results are shown in FIGS. 63A to 63D.

In separate experiments, BALB/C mice were administered with a single dose of 50 mg/kg 4M2-C12-hIgG1 ([1] of Example 2.2) or an equal volume of PBS. Blood samples were obtained after 96 hours, and analysed for numbers of different types of white blood cells using HM5 Hematology Analyser. Blood samples were also analysed for correlates hepatotoxicity and nephrotoxicity.

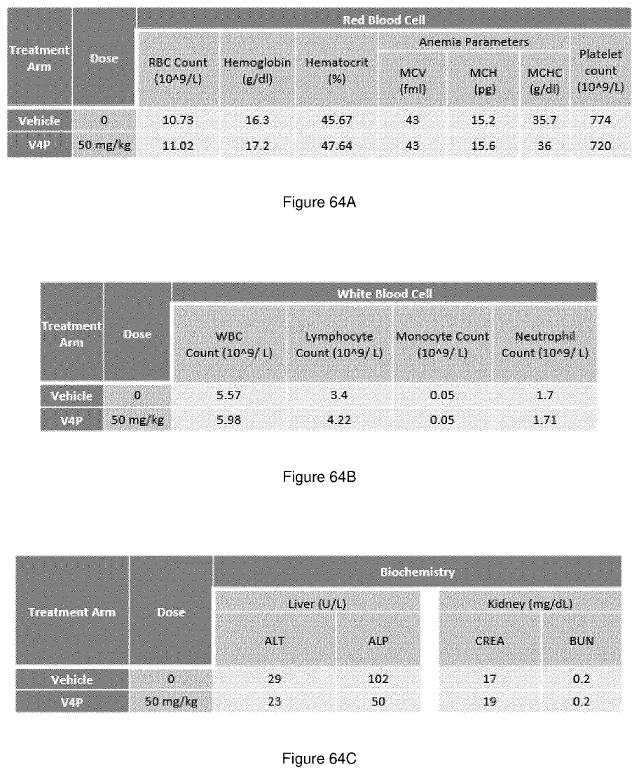

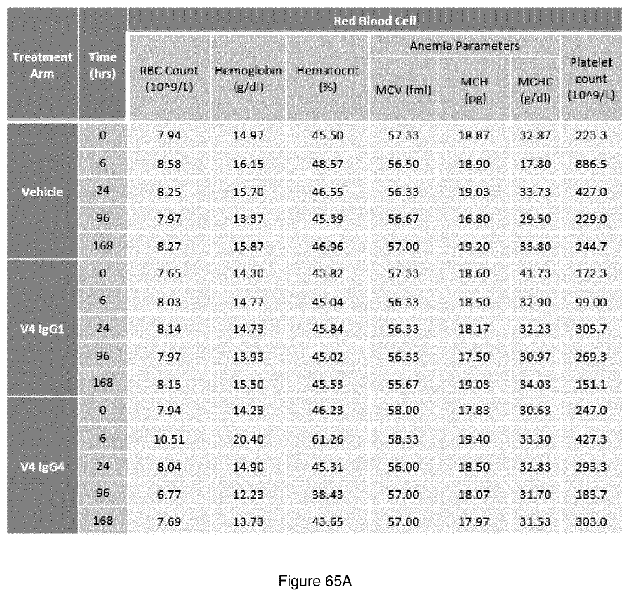

Representative results are shown in the tables of FIGS. 64A to 64C.

In further experiments, Sprague Dawley rats were administered with a single dose of 250 mg/kg 4M2-C12-hIgG1 ([1] of Example 2.2) or an equal volume of PBS. Blood samples were obtained at 6, 24, 96 and 168 hours, and analysed for numbers of different types of white blood cells using HM5 Hematology Analyser. Blood samples were also analysed for correlates hepatotoxicity, nephrotoxicity and pancreas toxicity.

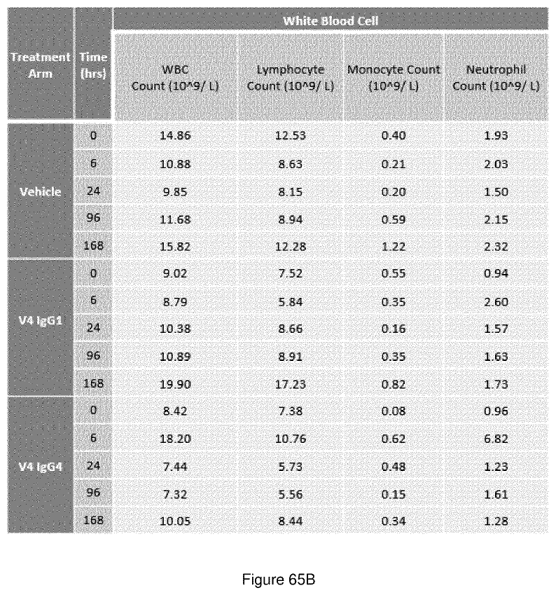

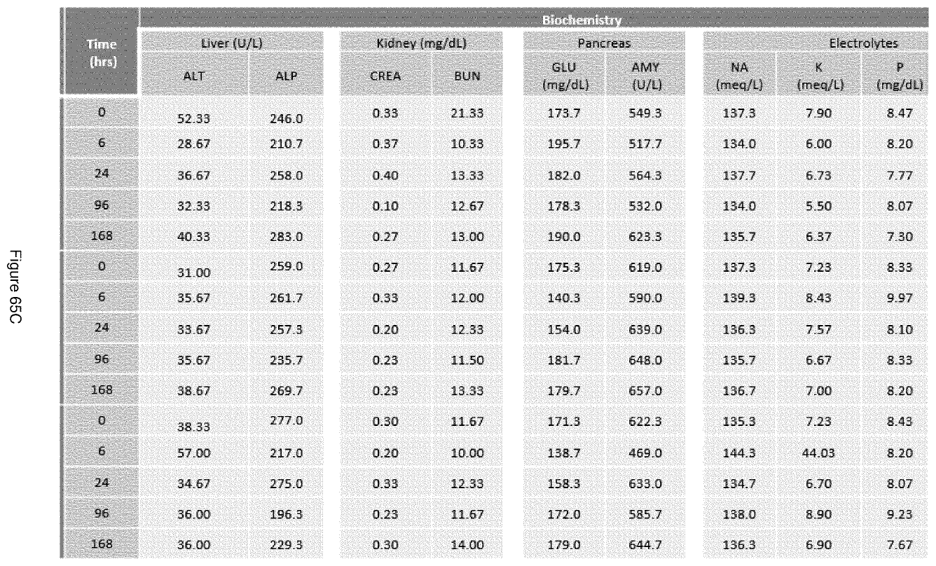

Representative results are shown in the tables of FIGS. 65A to 65C.

Administration of 4M2-C12-hIgG1 was not found to be associated with significant toxicity, and did not significantly alter numbers of cell types in blood.

### Example 17: Analysis of V4-C26 Antibody

### 17.1 Comparison of Binding to Human and Mouse VISTA for V4-C26, VSTB112 and mAb 13F3

Binding to human VISTA and mouse VISTA was analysed for anti-VISTA antibodies V4-C26 (i.e. molecule of Example 13), VSTB112 IgG1 (comprising VSTB112 HC (SEQ ID NO: 269)+VSTB112 LC (SEQ ID NO: 270) and mAb 13F3 (BioXCell Cat. No. BE0310).

Bio-Layer Interferometry (BLI) experiments were performed using the Octet QK384 system (ForteBio). All measurements were performed at 25° C.

Briefly, Anti-Penta-HIS (HIS1K) coated biosensor tips (Pall ForteBio, USA) were incubated for 60 sec in PBS buffer (pH 7.2) to obtain a first baseline, and tips were subsequently loaded for 120 sec with 270 nM HIS-tagged human VISTA or mouse VISTA, in PBS (pH 7.2).

After loading, biosensors were incubated for 60 sec in PBS buffer pH 7.2 to obtain a second baseline, and were then incubated for 120 sec with a 4 point, 2 fold dilution series of the test antibodies (concentrations: 250 nM, 125 nM, 62.5 nM and 31.3 nM) in PBS pH 7.2 to obtain association curves. Finally, the biosensors were incubated for 120 sec in PBS pH 7.2 to obtain dissociation curves.

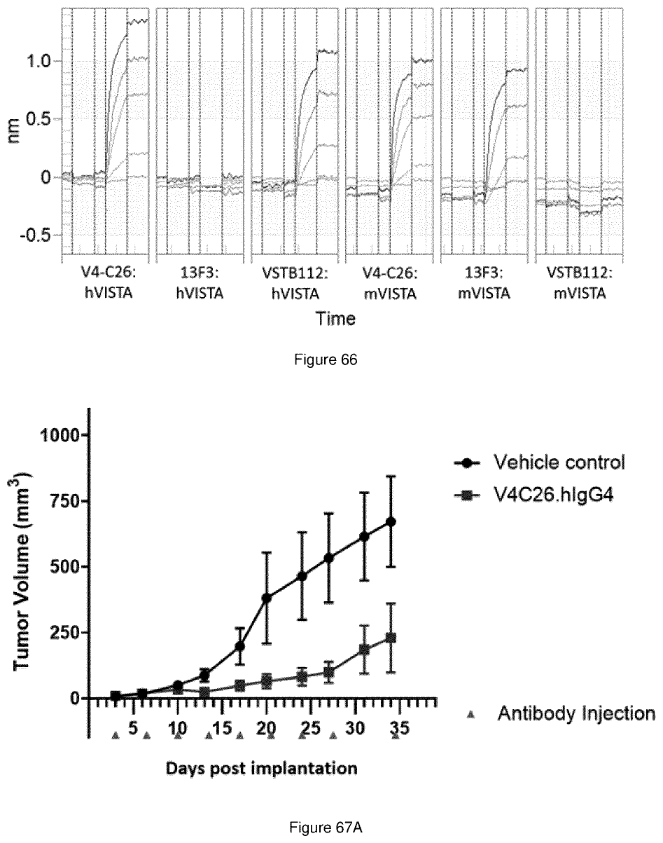

The results are shown in FIG. 66. V4-C26 was found to display binding to both human VISTA and mouse VISTA. By contrast, mAb 13F3 displayed binding to mouse VISTA but not human VISTA, and VSTB112 displayed binding to human VISTA but not mouse VISTA.

17.2 Tumour Growth Inhibition by V4-C26 hIgG4

The antigen-binding molecule V4-C26 hIgG4 comprising the heavy chain of SEQ ID NO:331 and the light chain of SEQ ID NO:317 was evaluated in a syngeneic cell line-derived mouse model of colon carcinoma for its ability to inhibit tumour growth in vivo.

CT26 cells were obtained from ATCC, and cultured in RPMI-1640 supplemented with 10% fetal bovine serum and 1% Pen/Strep, at 37° C. in a 5% CO₂ incubator.

CT26 cell-derived tumours were established by injecting 1×10⁵ CT26 cells subcutaneously into the right flanks of ˜6-8 week-old female BALB/c mice.

3 days post-implantation, and biweekly thereafter, mice were administered by intraperitoneal injection with 25 mg/kg V4-C26 hIgG4, or an equal volume of vehicle as a control condition (8 mice per treatment group).

Tumor volume was measured 3 times a week using a digital caliper, and calculated using the formula [L×W2/2]. The study end point was considered to have been reached once the tumours of the control arm measured >1.5 cm in length.

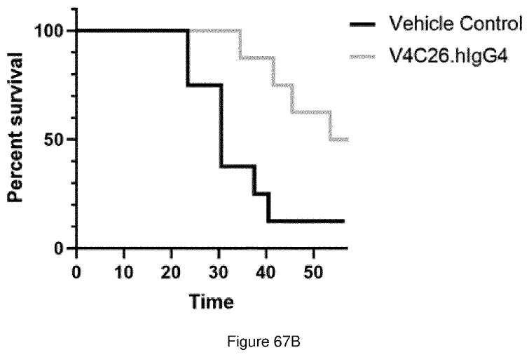

The results are shown in FIGS. 67A and 67B. Mice administered with V4-C26 hIgG4 displayed significant inhibition of tumour growth and improved survival relative to mice administered with vehicle.

### 17.3 Distribution of VISTA Expression

### 17.3.1 VISTA is Predominantly Expressed on Myeloid-Derived Cells in Healthy Tissues

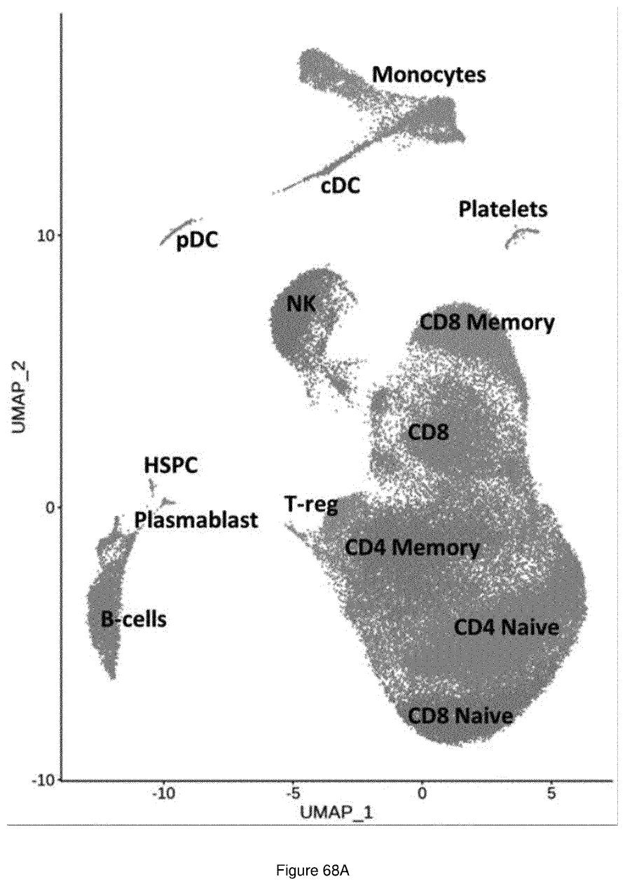

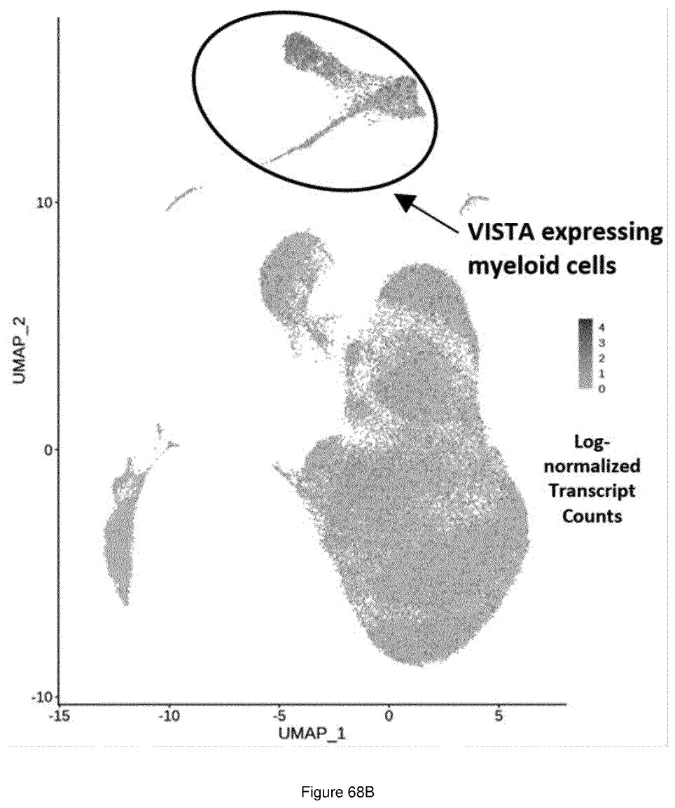

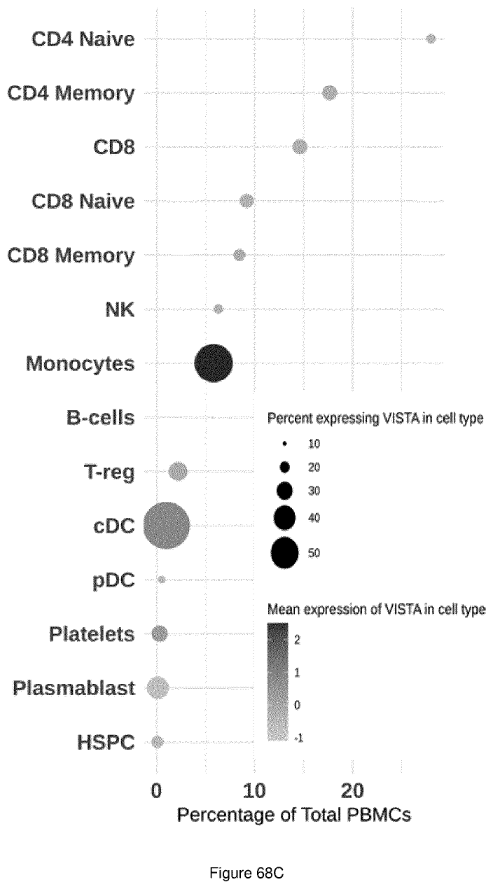

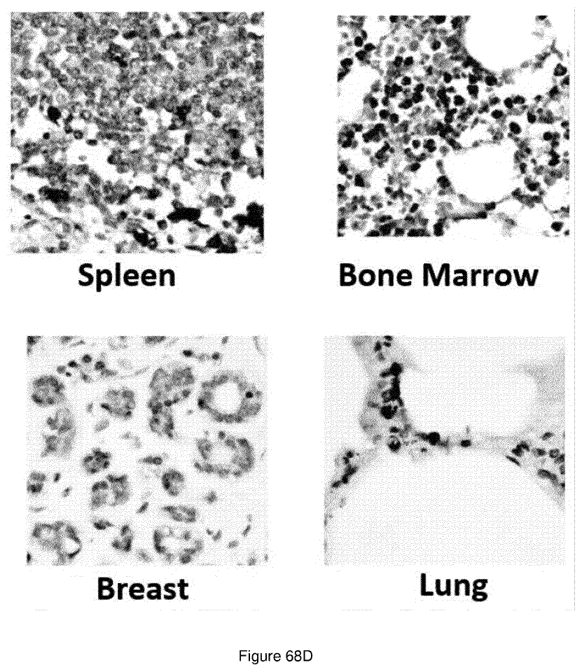

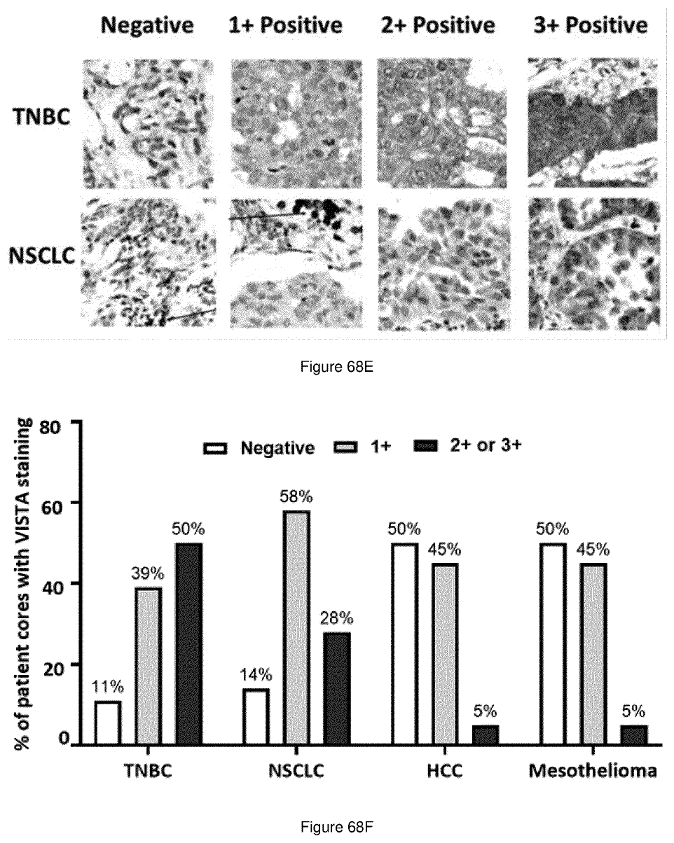

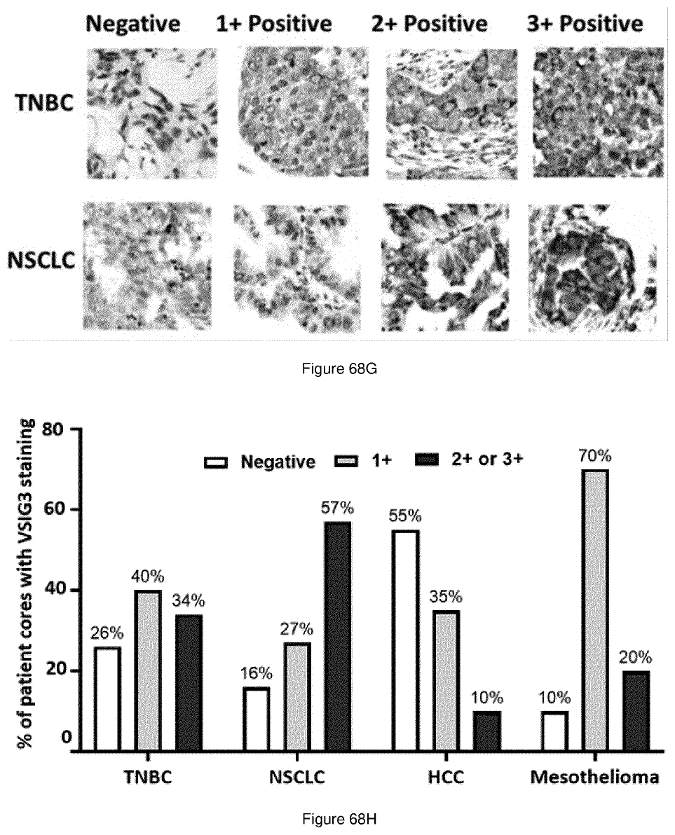

The distribution of VISTA expression was studied in healthy tissues to evaluate the optimal strategy for a VISTA antagonist. A single cell (sc) RNA-seq dataset from 10× Genomics comprising 68,000 PBMCs [www.10xgenomics.com/single-cell-gene-expression/datasets] was analysed. This revealed the presence of 14 major cell clusters (FIG. 68A). Although low levels of VISTA transcripts were identified in many cell populations, high expression was confined to myeloid-derived monocytes and dendritic cells in healthy human donors, with limited expression in T cells (FIGS. 68B, 68C).

VISTA protein expression in healthy human tissues was further characterized using immunohistochemistry (IHC) on formalin fixed paraffin embedded (FFPE) tissue microarray (TMA) sections (FIG. 68D). The highest VISTA levels were detected in lymphoid organs (e.g. spleen and bone marrow) and tissues with significant infiltration by leukocytes (e.g. breast and lung). The distribution of VISTA across healthy tissues and diverse immune cells strongly supports the need to antagonize VISTA without depleting VISTA expressing cells for optimal therapeutic benefit and tolerable safety.

### 17.3.2 Solid Tumours Including TNBC and NSCLC Show Significant Expression of VISTA and VSIG3

The expression of VISTA and VSIG3 was assessed via immunohistochemistry in formalin fixed paraffin embedded (FFPE) tissue microarray (TMA) sections of four solid cancers, including triple negative breast cancer (TNBC), non-small cell lung cancer (NSCLC), hepato-cellular carcinoma (HCC) and mesothelioma. Immunohistochemistry used 4M2-C12-mIgG2a (which is formed of the polypeptides of SEQ ID NOs: 248 and 250) or an anti-VSIG3 antibody.

TNBC and NSCLC patients showed the highest VISTA expression, with 89% and 85%, of the cores showing moderate-high staining intensity, respectively (FIG. 68E, 68F). The expression of VSIG3 was evaluated further. VSIG3 showed moderate to high staining in mesothelioma (90%), NSCLC (84%), and TNBC (74%) (FIG. 68G, 68H). The observed high expression of both VISTA and VSIG3 in TNBC and NSCLC, is consistent with reports that the co-inhibitory function of VISTA may be mediated through VISTA-VSIG3 interactions suppressing T cell function (Wang et al., Immunology (2019) 156:74-85) and suggests that these could be priority indications to investigate the benefits of VISTA antagonism.

17.4 Analysis of Properties of V4-C26 hIgG4

V4-C26 was developed as an IgG4 isotype comprising the heavy chain of SEQ ID NO:331 and the light chain of SEQ ID NO:317.

### 17.4.1 Analysis of ADCC and CDC Functionality

To rule out the potential for V4-C26 hIgG4 to cause ADCC and CDC reactions, its binding affinity to the respective FcγRIII and C1q proteins was evaluated. No binding was observed between FcγRIII and C1q proteins by ELISA (FIGS. 69A and 69B), which would preclude ADCC or CDC mechanisms depleting VISTA expressing cells.

This feature makes V4-C26 hIgG4 distinct from previously developed VISTA targeting antibodies that have used an IgG1 Fc isotype, known to trigger cell depletion by ADCC and CDC.

17.4.2 Biophysical Properties of V4-C26 hIgG4

Binding specificity of V4-C26 hIgG4 for VISTA was measured by ELISA.

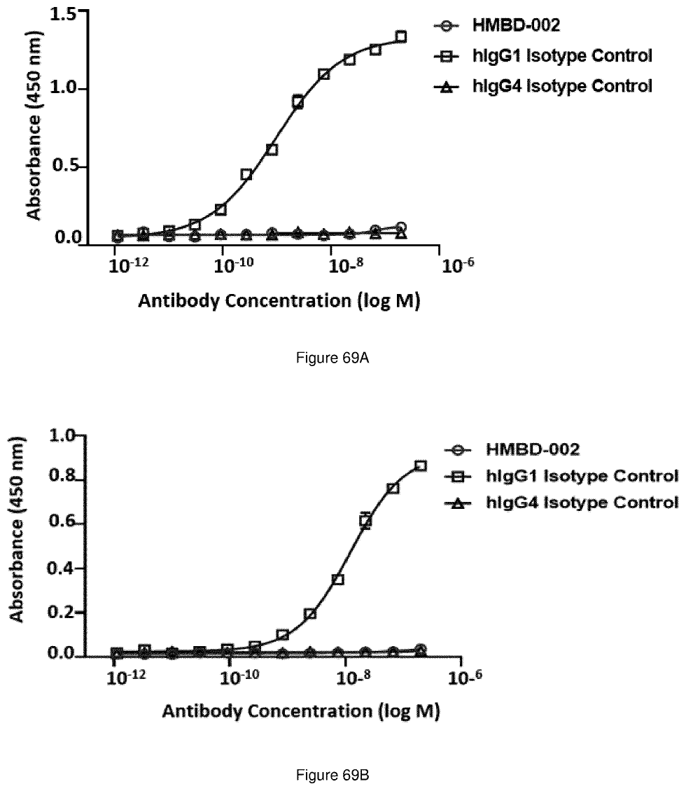

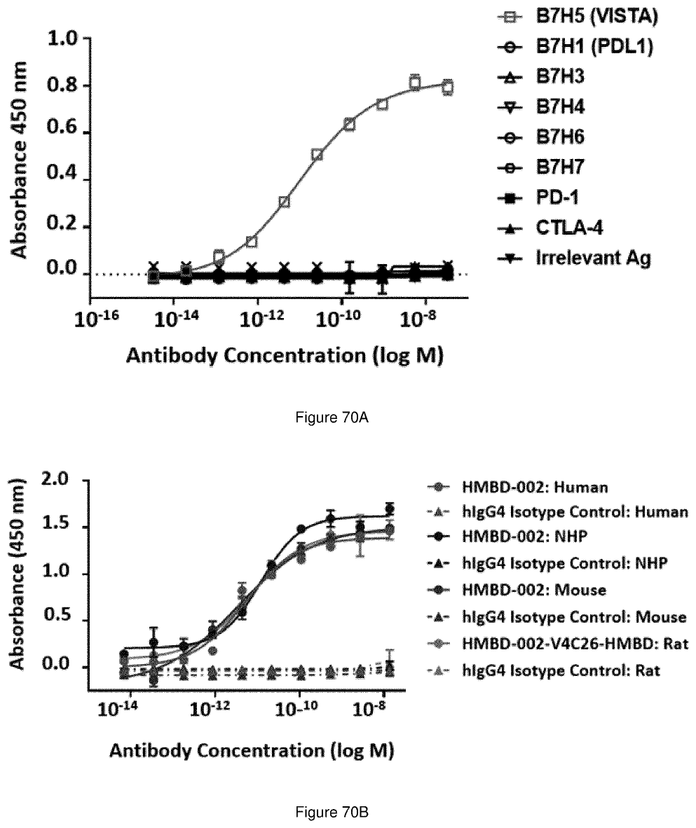

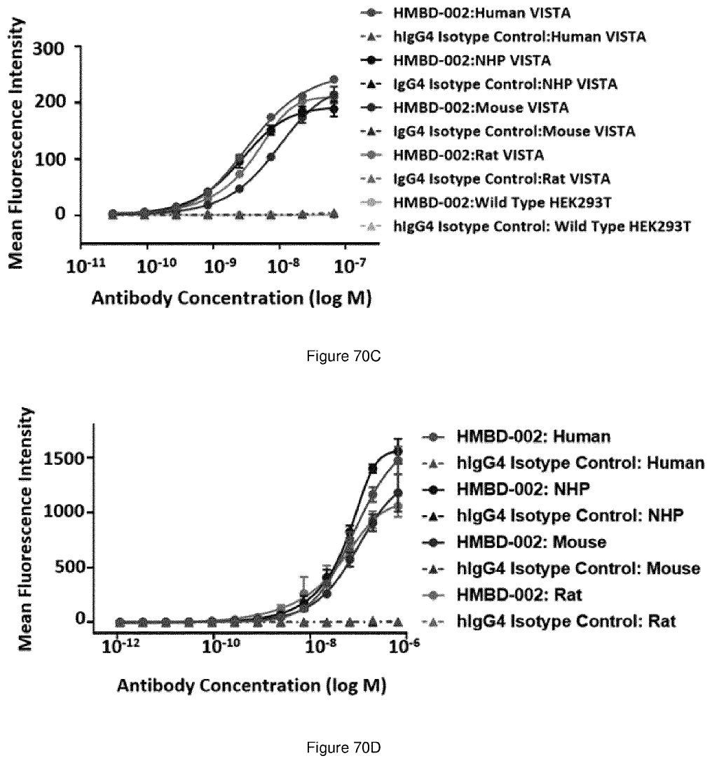

FIG. 70A shows that V4-C26 hIgG4 displays highly specific binding to VISTA among the other related members of the B7 family (B7H1/PDL-1, B7H3, B7H4, B7H6, B7H7), as well as PD-1 and CTLA-4.

To support the use of rodent and non-human primate (NHP) species for efficacy, safety and PK models, the ability of V4-C26 hIgG4 to bind to VISTA orthologs was assessed using ELISA and SPR (Biacore).

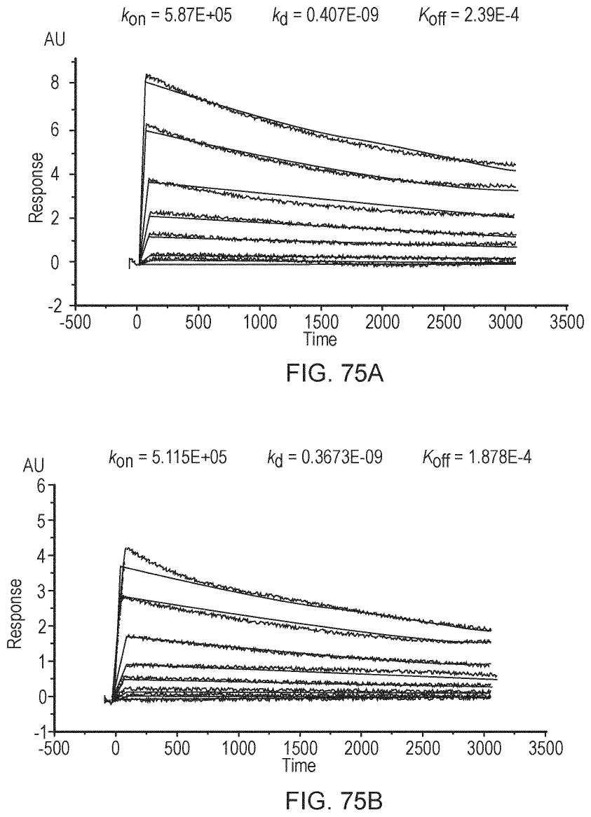

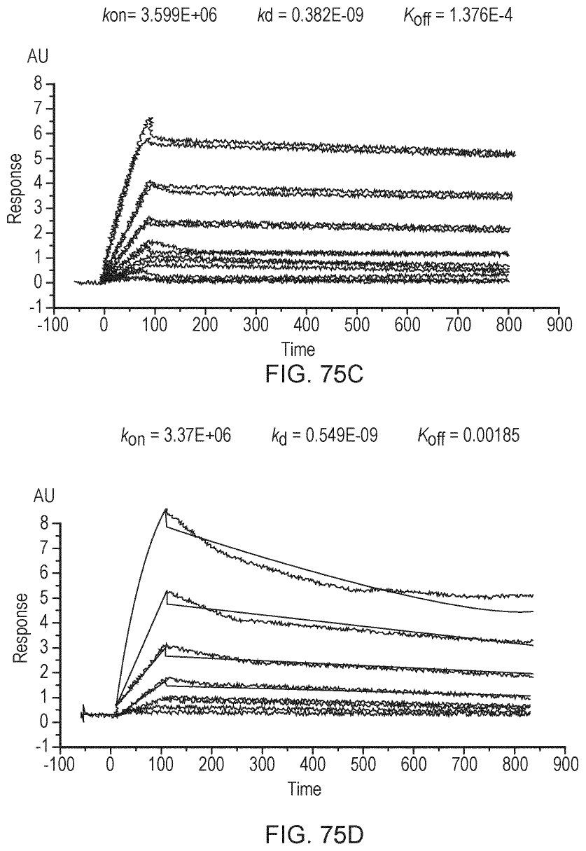

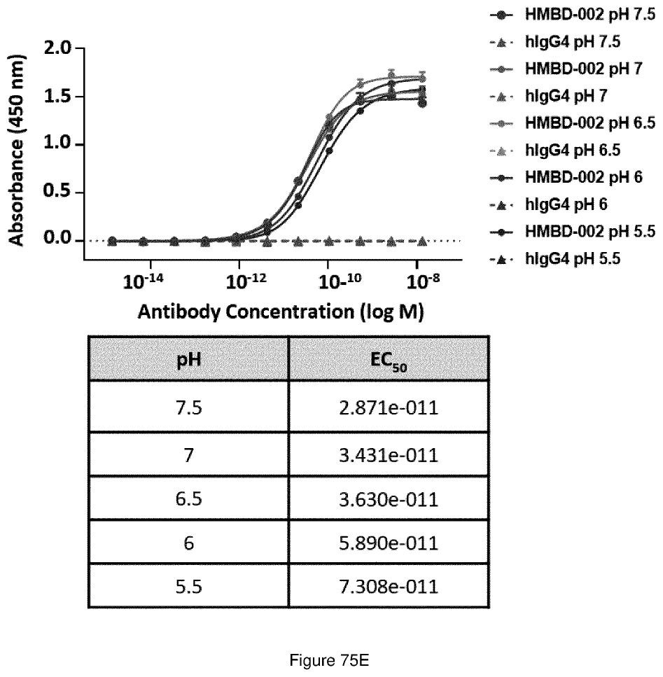

FIG. 70B shows that V4-C26 hIgG4 demonstrates dose-dependent binding to human, NHP, rat and mouse VISTA-HIS protein by ELISA with an EC₅₀ of 5.117 pM, 12.15 pM, 6.689 pM, and 3.549 PM, respectively. V4-C26 hIgG4 was also observed to bind to human, NHP, rat and mouse VISTA orthologs with similar picomolar affinities (Kd) of 407 pM, 367 pM, 382 pM, and 549 pM respectively using SPR (Biacore)—see FIGS. 75A to 75D.

Cell surface binding of V4-C26 hIgG4 to HEK293T cells expressing recombinant VISTA orthologs, as well as to myeloid cells within PBMC of relevant pre-clinical species, was further confirmed by FACS.

FIG. 70C shows that V4-C26 hIgG4 showed dose-dependent binding to VISTA orthologs of all species tested, with comparable EC₅₀ values of 3.738 nM, 2.571 nM, 4.133 nM and 8.94 nM for human, NHP, rat, and mouse VISTA expressing HEK293T cells, respectively. There was no non-specific binding observed to wild type HEK293T cells, which do not express VISTA.

FIG. 70D shows that V4-C26 hIgG4 also showed comparable dose-dependent binding to endogenous VISTA expressed on the myeloid cells of all pre-clinical species tested, with EC₅₀ of 108 nM, 67.6 nM, 48.6 nM and 111 nM for human, NHP, rat, and mouse VISTA, respectively.

Some tumour environments have been reported to be hypoxic, characterized by relatively low pH. As VISTA contains many exposed histidine residues that are susceptible to protonation and may affect antibody binding, we evaluated the effect of pH on V4-C26 hIgG4 binding. FIG. 75E shows that V4-C26 hIgG4 was observed to bind VISTA with comparable affinity at pH 5.5-7.5, as assessed by ELISA, confirming that V4-C26 hIgG4 binds to VISTA across the range of physiologically relevant pH, and that the binding site is distinct from the histidine-rich regions.

Thus, the binding of V4-C26 hIgG4 is highly selective and maintained even in low pH conditions that may represent those in the tumour microenvironment.

17.5 Ability of V4-C26 hIgG4 to Block VISTA Functionality
17.5.1 V4-C26 hIgG4 Modulates Myeloid Function

To investigate the effect of VISTA blockade on myeloid cells that express the highest levels of VISTA, V4-C26 hIgG4 treatment was evaluated in several in vitro models of myeloid function.

First, VISTA blockade by V4-C26 hIgG4 was assessed for its effect on the function of human monocytic MDSCs. Previous studies have reported that MDSCs contribute significantly to suppression of T cell function in the TME (L. Wang, et al., Oncoimmunology 7, e1469594 (2018)). Monocytes differentiated to MDSCs for 7 days with GM-CSF and IL-6 were co-cultured with autologous PBMCs. T cells were then stimulated with an anti-CD3 antibody and IFN-γ levels in the culture supernatant were measured by ELISA.

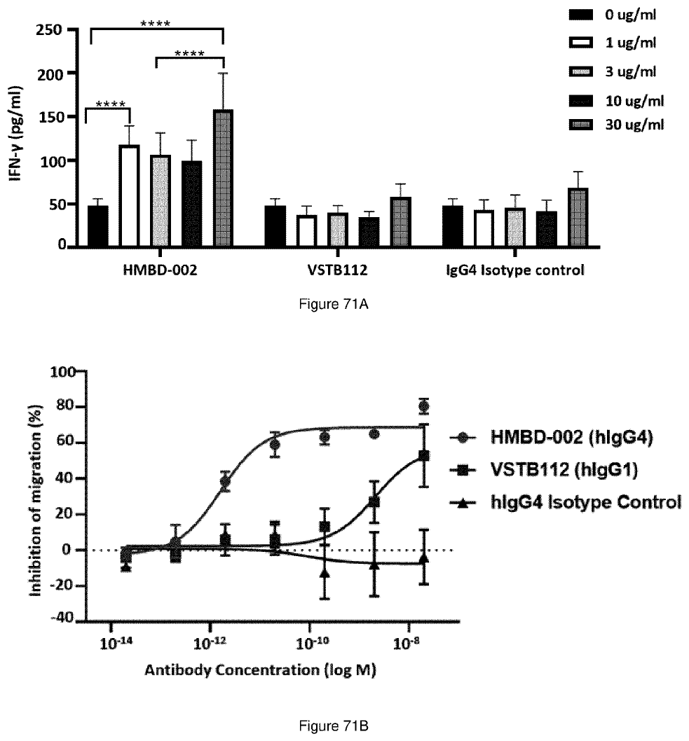

FIG. 71A shows that addition of V4-C26 hIgG4, but not VSTB112, successfully reversed MDSC-mediated T cell suppression in response to anti-CD3 stimulation as indicated by the enhanced levels of IFN-γ.

Granulocytes such as neutrophils are an integral part of the innate immune response but can adversely affect cancer progression. Although it is challenging to model the function of granulocytic (g)-MDSCs in vitro, it is known that g-MDSCs can arise from neutrophils that have infiltrated the tumour microenvironment (G. E. Kaiko, et al., Immunology 123, 326-338 (2008)). The effect of V4-C26 hIgG4-mediated VISTA blockade on neutrophil chemotaxis was explored using a transwell assay. In this assay, neutrophils migrate between chambers towards a physiologically relevant chemoattractant, C5a, and the percent of cells that have migrated to the lower chamber is then quantified via a luminescence readout.

FIG. 71B shows that V4-C26 hIgG4 potently inhibited neutrophil migration in a dose-dependent manner. VSTB112, in contrast, could only suppress neutrophil migration at much higher concentrations. Collectively these results demonstrate that V4-C26 hIgG4 potently neutralizes VISTA on myeloid cells leading to the increased secretion of pro-inflammatory cytokines and decreased migration of neutrophils.

17.5.2 V4-C26 hIgG4 Polarises the Immune Cell Milieu Towards a T_{H}1/T_{H}17 Immune Response

To investigate the functional effect of VISTA blockade by V4-C26 hIgG4 in more complex in vitro models of immune activation, an allogenic Mixed Lymphocyte Reaction (MLR) assay was used. Briefly, PBMCs from 5 independent healthy human donors were mixed pairwise to model a self/anti-self immune response and were then cultured for up to 96 hours in the presence or absence of V4-C26 hIgG4 before supernatants were analyzed for cytokine levels. The transcriptome of the cells was also analyzed using bulk RNA-seq. Cytokine levels in the supernatant were quantified by Luminex assay.

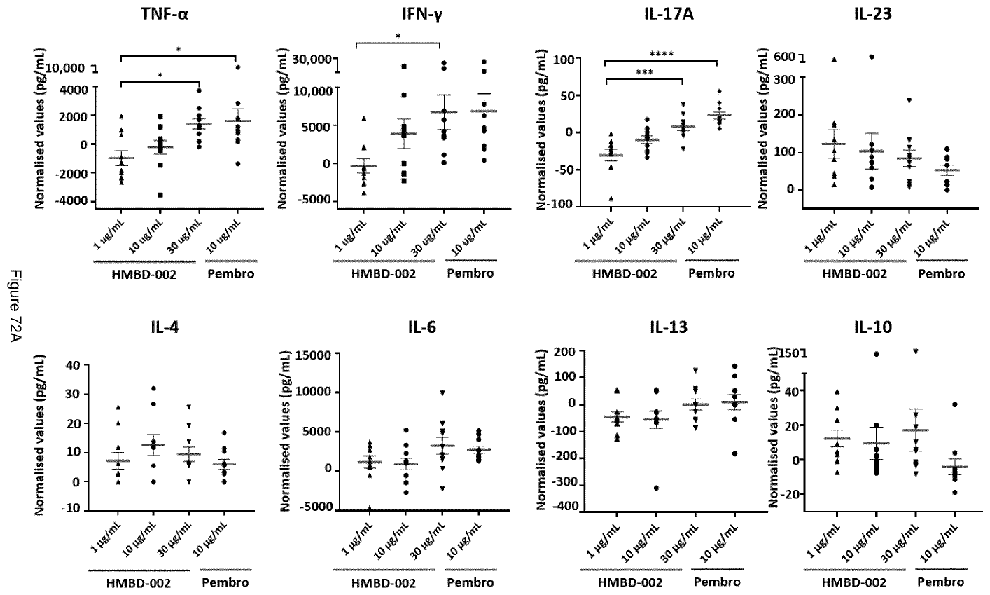

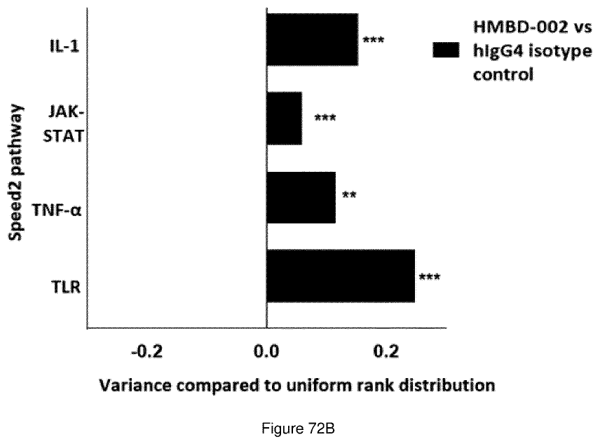

FIG. 72A shows that V4-C26 hIgG4 induced a significant dose dependent increase in the levels of IFN-γ, TNF-α and IL-17A at 96 hours, comparable to the effect seen with PD-1/PD-L1 blockade by an anti-PD-1 antibody, Pembrolizumab, but no significant changes in the levels of IL-4, IL-10 and IL-13 (Th2 cytokines) or IL-6. These changes in cytokine levels are indicative of a shift to a Th1/Th17 response.

This conclusion was further supported by bulk RNA-seq that highlighted an enrichment of transcript levels in genes associated with TLR, TNF-α, IL-1, JAK-STAT and IL-17 signalling pathways (FIGS. 72B, 72C) along with an increase in transcript levels of Th1 associated genes such as IFN-γ, TNF-α and IL-12A and a decrease in transcript levels of Th2 associated genes IL-4, IL-10, IL-13 and IL-9 (FIG. 72D).

Collectively, these results show that V4-C26 hIgG4 blockade of VISTA can polarize the immune cell milieu toward an enhanced Th1/Th17 immune response. This is consistent with murine VISTA knockout models that lead to psoriasis and experimental autoimmune encephalomyelitis (EAE) that have previously been reported to be characterized by Th1/Th17 responses (N. Li et al., Sci Rep-uk 7, 1485 (2017)).

17.6 V4-C26 hIgG4 Treatment Induces Strong Anti-Tumour Responses in Multiple Immune Competent Murine CDX Models of Solid Tumours

To explore the pro-inflammatory and anti-tumorigenic effects of V4-C26 hIgG4 in vivo, tumour growth inhibition studies were conducted in multiple solid tumour models that included a syngeneic murine cell-derived xenograft (CDX) subcutaneous model of colon cancer (CT26), a checkpoint inhibitor-resistant orthotopic CDX model of VISTA expressing breast cancer (4T1) and CD34 engrafted humanized mouse models of human lung cancer (A549) and colorectal cancer (HCT15).

First, Balb/c mice were subcutaneously implanted (right flank) with CT26 tumours and treated biweekly with 500 μg (˜25 mg/kg) of V4-C26 hIgG4 (intraperitoneal) from 3 days post implantation.

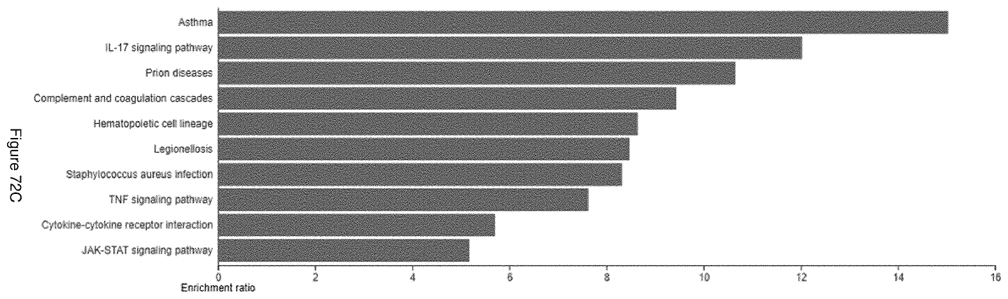

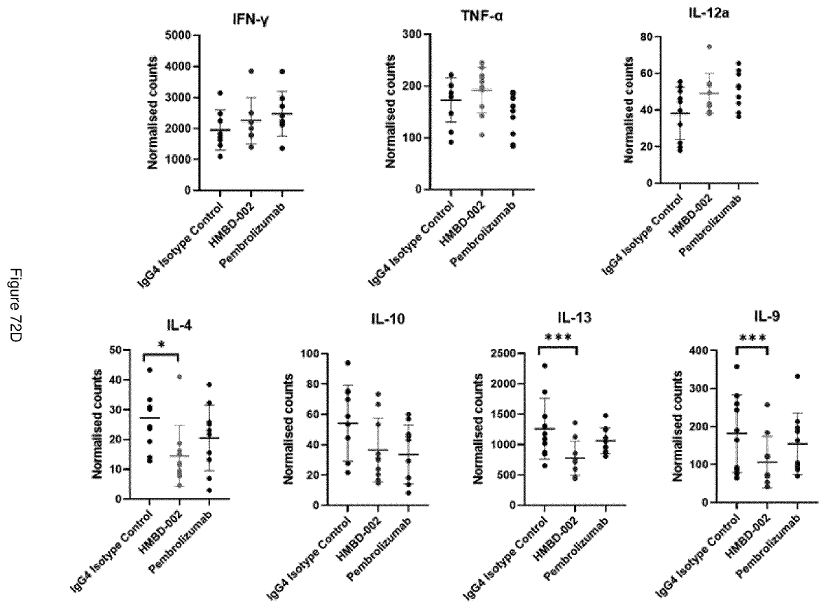

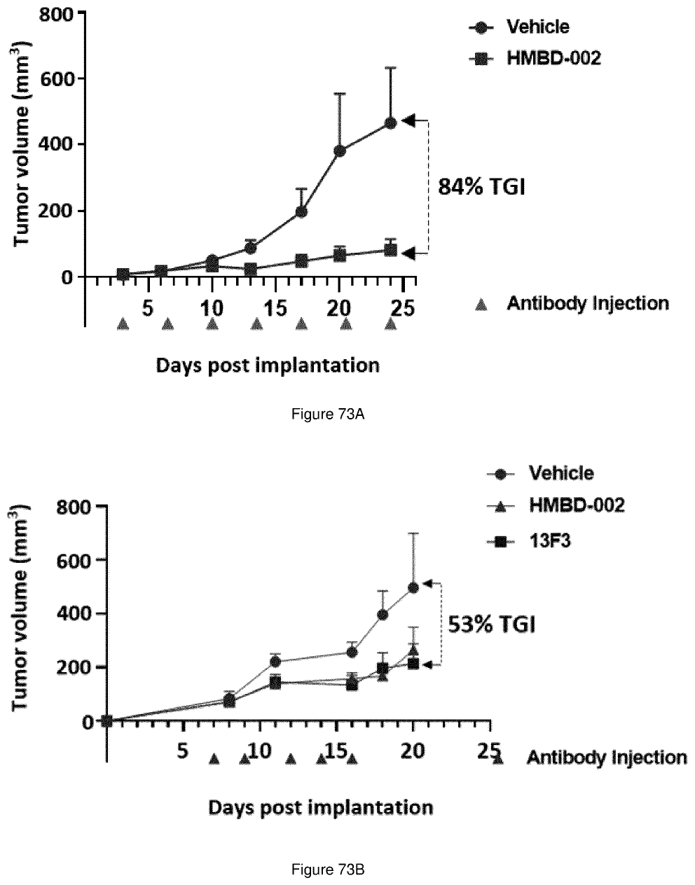

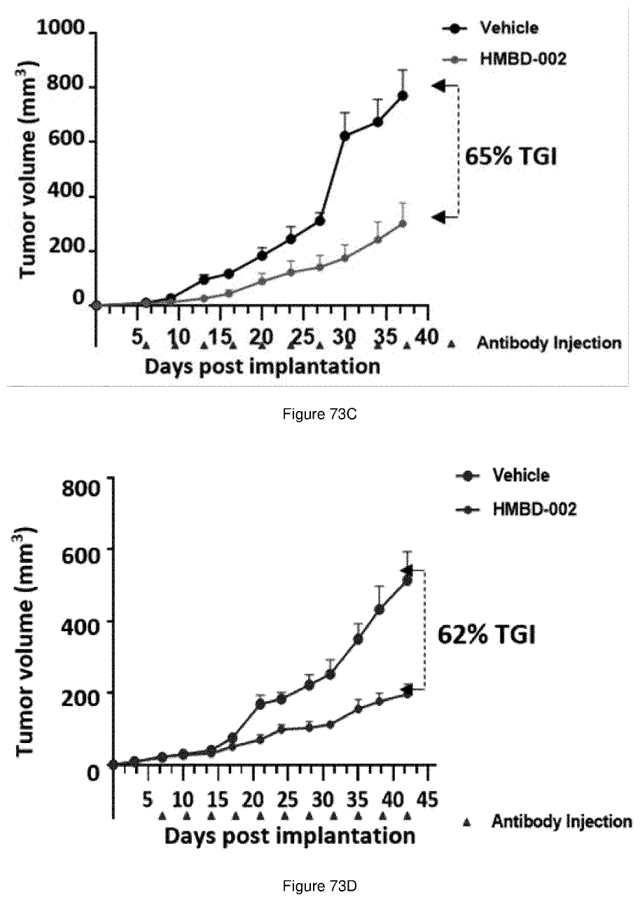

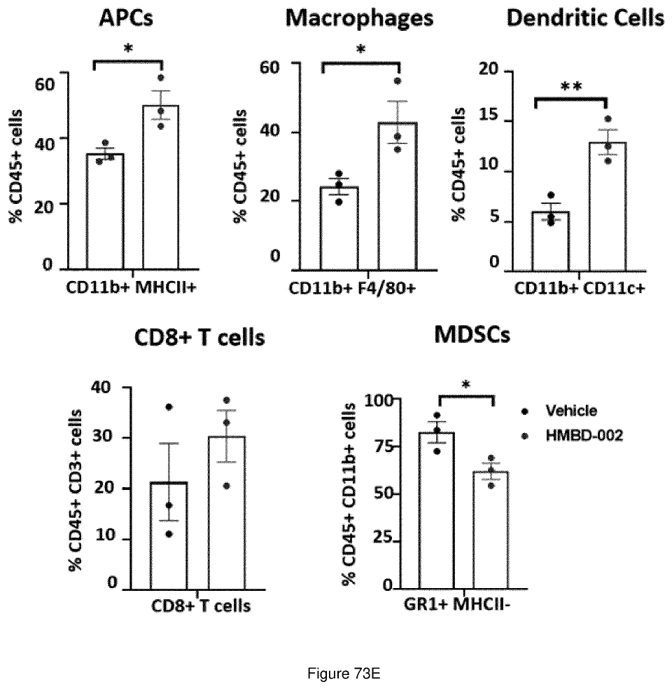

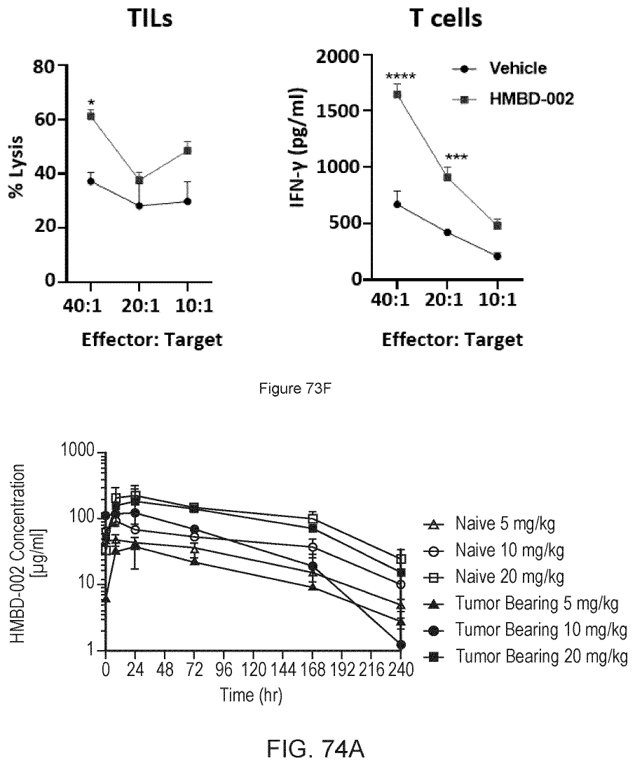

FIG. 73A shows that V4-C26 hIgG4 demonstrated significant single agent efficacy with 84% inhibition of tumour growth (TGI) compared to vehicle.

Secondly, a murine orthotopic model of breast cancer was established by implanting 4T1 cells overexpressing VISTA in the mammary fat pads of Balb/c mice. Mice were treated intratumorally with 50 μg of either V4-C26 hIgG4 or the anti-mouse VISTA antibody 13F3 on days 7, 9, 12, 14, 16 post-implantation. 13F3 is frequently used as a mouse surrogate for studying the in vivo efficacy of anti-VISTA antibodies.

FIG. 73B shows again that V4-C26 hIgG4 showed significant TGI (53%), which was comparable to that of 13F3 suggesting the two antibodies may share a common mechanism of action.

Finally, the efficacy of V4-C26 hIgG4 was tested in humanized mice engrafted with CD34+ cord blood hematopoietic stem cells. These humanized mice received a GM-CSF/IL3 boost that stably reconstituted multiple human cell lineages such as T, B and myeloid cells in organs and blood, two days prior to tumor implantation (Table S1). After reconstitution for 3-4 months, mice were subcutaneously implanted with either human HCT15 colorectal cancer cells or human A549 lung cancer cells and treated biweekly (intraperitoneal with 500 μg (˜25 mg/kg) of either V4-C26 hIgG4 or vehicle control starting at 5 days post implantation.

FIGS. 73C and 73D show that, as observed in the syngeneic models, V4-C26 hIgG4 showed significant single agent anti-tumour efficacy of 65% and 62% TGI in these HCT15 colorectal and A549 lung humanized CDX models, respectively.

Thus, V4-C26 hIgG4 demonstrates strong anti-tumour responses as a single agent in multiple CDX models of solid tumours. The similar tumour inhibition observed after V4-C26 hIgG4 treatment of both fully immune-competent murine tumour models and murine tumour models recapitulating the human immune compartment, suggests a conserved, and potentially translatable, mechanism of action shared between mouse and human anti-tumour immune responses.

17.7 V4-C26 hIgG4 Treatment Remodels the Tumour Microenvironment of Murine CDX Models. Increasing Activated Effector Immune Cells and Decreasing Suppressive Cells

To understand the mechanism of the anti-tumour efficacy observed for V4-C26 hIgG4 in murine models, tumours from the syngeneic CT26 colon cancer models were further profiled using FACS.

FIG. 73E shows that V4-C26 hIgG4 treatment was observed to significantly increase the percentage of CD11b+ MHCII+ (antigen presenting cells), CD11b+ F4/80+ (macrophages) and CD11c+ (DCs) in the tumour microenvironment. An increase in CD8+ T cells was also observed, although this was not statistically significant. In contrast, the frequency of broadly-suppressive MDSCs (CD11b+GR1+MHCII−) was significantly lower in the tumours of treated mice compared to vehicle control.

To further assess the mechanism by which these changes in immune cell numbers were associated with the functional state of tumour infiltrating lymphocytes (TILs), we performed an antigen recall assay by co-culturing CT26 cells and isolated TILs from the CDX models in vitro.

FIG. 73F shows that, 72 hours after coculture, TILs isolated from tumours treated with V4-C26 hIgG4 showed significantly higher lysis of CT26 cells. This increased activity was further confirmed with an ex vivo co-culture assay of infiltrating T cells with CT26 cells, where T cells from treated mice showed significantly higher IFN-γ levels, as measured by ELISA, compared to untreated mice.

These results suggest that VISTA blockade by V4-C26 hIgG4 increases the levels of inflammatory effector cells with concomitant decrease of immunosuppressive MDSCs in the tumour microenvironment and enhances the antigen-specific cytotoxic activity of TILs, likely contributing to the anti-tumour efficacy of V4-C26 hIgG4. V4-C26 hIgG4 is able to remodel the tumour microenvironment to an anti-tumorigenic, pro-inflammatory phenotype, consistent with the primary mechanism of action being manipulation of the highly VISTA-positive myeloid compartment.

17.8 V4-C26 hIgG4 Exhibits Favourable Pharmacokinetic Profiles in Multiple Species

Therapeutic antibodies should possess half-lives in plasma compatible with appropriate dosing regimens.

Previous anti-VISTA antibodies have demonstrated poor PK characterized by rapid serum clearance (R. J. Johnston et al., Nature 574, 565-570 (2019)), which may be explained by Fc-effector functions such as ADCC, especially of neutrophils, leading to rapid cell turnover of VISTA expressing cells and causing a significant antibody sink. The pharmacokinetic profile of V4-C26 hIgG4 was therefore evaluated in healthy and tumour-bearing mice, and healthy rats and NHP.

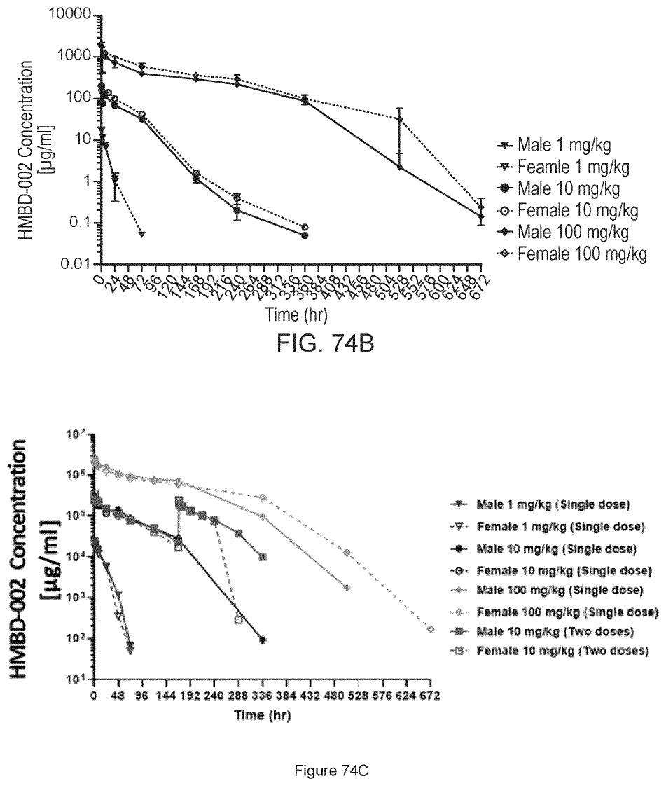

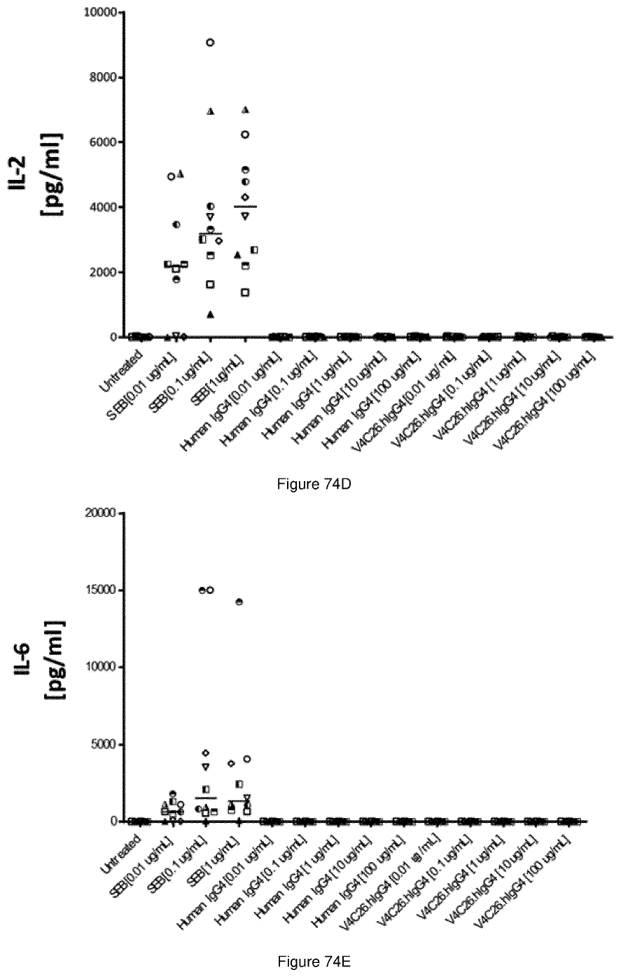

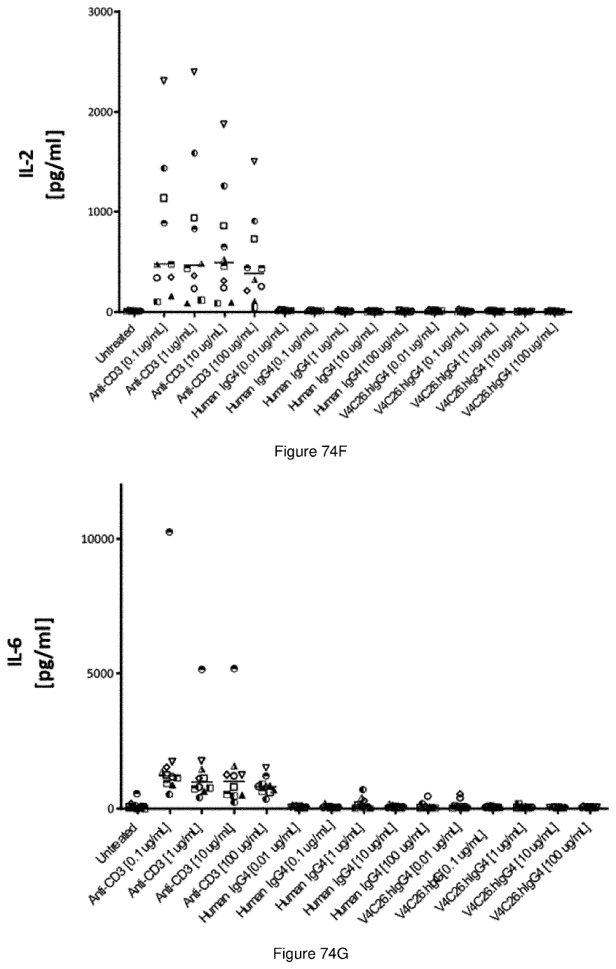

FIG. 74A shows representative pharmacokinetic profiles for tumour-bearing and non-tumour bearing Balb/c mice that had been dosed intraperitoneally with increasing doses of V4-C26 hIgG4 (5-20 mg/kg). Non-tumour bearing mice demonstrated linear PK, however, profiles in tumour bearing mice were non-linear, likely due to the higher levels of the target and increased target mediated drug disposition (TMDD). V4-C26 hIgG4 serum half-life in the tumour bearing mice was calculated as 26.9 to 57.2 hours across doses, while in the non-tumour bearing mice serum half-life was 61.1 to 80.8 hours.

In Sprague-Dawley rats and cynomolgus monkeys, the PK profile was determined from measurements in both male and female animals. V4-C26 hIgG4 was administered at single doses of 1 mg/kg, 10 mg/kg, and 100 mg/kg, as an intravenous (IV) bolus infusion into the tail vein in rats, and as an IV bolus infusion into the peripheral vein in monkeys. In both species, the PK profiles of V4-C26 hIgG4 did not differ between the genders and dose proportional increases in exposure were observed for Cmax values across all doses. Serum half-life for V4-C26 hIgG4 increased with each dosing group. The mean observed half-life across both genders in rat were 6.3, 25.5, and 67.5 hours for 1, 10, and 100 mg/kg, respectively, whereas the mean observed half-life across both genders in cynomolgus monkey were 8.6, 41.3, and 34.9 hours for 1, 10, and 100 mg/kg, respectively (FIGS. 74B, 74C).

Together, these results demonstrate that V4-C26 hIgG4 has a favourable PK profile in multiple species without the rapid clearance that has been noted for other anti-VISTA antibodies with a depleting IgG1 Fc domain.

Optimal therapeutic antibodies should demonstrate minimal toxicity to normal tissues and avoid detrimental side effects such as cytokine release syndrome (as reported for other IgG1 isotype anti-VISTA depleting antibodies at sub therapeutic doses (NCT02671955)). As V4-C26 hIgG4 shows species conserved binding of VISTA orthologs, animals from the above PK studies were also monitored for adverse effects of V4-C26 hIgG4 dosing, as part of concurrent tolerability studies. After a single IV injection in Sprague-Dawley rats and cynomolgus monkey, both species showed no treatment related morbidity/mortality or clinical signs, and no treatment related changes in body weight, food consumption, clinical chemistry, or hematology parameters that continued for the duration of the 28-day observation period.

Additionally, ex vivo cytokine release assays were conducted with human whole blood and isolated PBMCs from healthy donors, to assess the potential immunotoxicity of V4-C26 hIgG4 by evaluating the levels of IL-2, IL-4, IL-6, IL-10, TNF-α and IFN-γ in culture supernatants using a cytometric bead array approach. Cytokine levels were measured after stimulation for 24 hours with either V4-C26 hIgG4, a positive control (anti-CD3 or staphylococcal enterotoxin B), or a negative isotype control in soluble stimulation format. V4-C26 hIgG4 did not elicit any significant cytokine release in blood or in purified PBMCs (FIGS. 74D to 74G).

Together these results demonstrate that V4-C26 hIgG4 is well tolerated in rats and cynomolgus monkeys and further suggests a low risk for potential immunotoxicity caused by cytokine release.

17.9 Tumour Growth Inhibition by V4-C26 hIgG4 and Anti-PD-1

### A549 Cell Model

A549 cells were obtained from ATCC. Cells were harvested at 70-80% confluency, washed three times with 15 ml of PBS, counted using a hemocytometer and reconstituted to the concentration of 5×10⁷ cells/ml in PBS.

A549 cell-derived tumours were established by implanting 5×10⁶ A549 cells (in a total volume of 0.1 ml) subcutaneously into the right flank of 6-8 week-old CD34 engrafted humanized HiMice mice.

3 days post implantation, and biweekly thereafter for 6-7 weeks, mice were administered intraperitoneally with doses of:
500 μg of V4-C26 hIgG4 (HMBD-002) (equivalent to 25 mg/kg) in a total volume of 0.2 ml.
200 μg of anti-human PD-1 antibody (equivalent to 10 mg/kg) in a total volume of 0.2 ml.
500 μg of V4-C26 hIgG4+200 μg of anti-human PD-1 antibody in a total volume of 0.2 ml.

Tumour volume was measured over the course of the treatment.

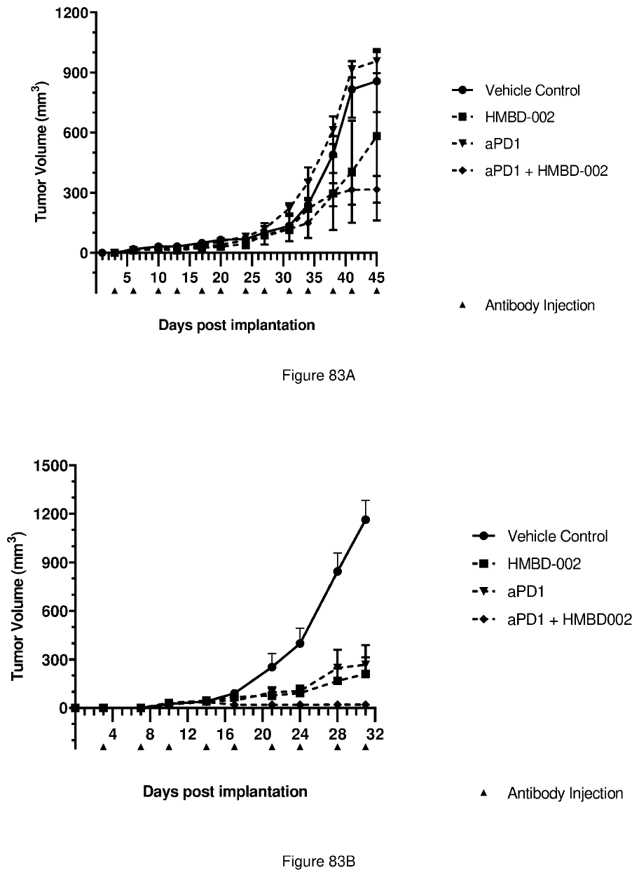

The results are shown in FIG. 83A. Combination therapy with anti-VISTA antibody V4-C26 hIgG4 (HMBD-002) and anti-PD-1 inhibited tumour growth to a greater extent than either agent alone.

### CD26 Cell Model

CT26 cells were obtained from ATCC. Cells were harvested at 70-80% confluency, washed three times with 15 ml of PBS, counted using a hemocytometer and reconstituted to the concentration of 5×10⁶ cells/ml in PBS.

CT26 cell-derived tumours were established by implanting 1×10⁵ CT26 cells (in a total volume of 0.1 ml) subcutaneously into the right flank of 6-8 week-old Balb/c mice.

3 days post implantation, and biweekly thereafter for 4-5 weeks, mice were administered intraperitoneally with doses of:
500 μg of V4-C26 hIgG4 (HMBD-002) (equivalent to 25 mg/kg) in a total volume of 0.2 ml.
200 μg of anti-mouse PD-1 antibody (equivalent to 10 mg/kg) in a total volume of 0.2 ml.
500 μg of V4-C26 hIgG4+200 μg of anti-mouse PD-1 antibody in a total volume of 0.2 ml.

Tumour volume was measured over the course of the treatment.

The results are shown in FIG. 83B. Combination therapy with anti-VISTA antibody V4-C26 hIgG4 (HMBD-002) and anti-PD-1 inhibited tumour growth to a greater extent than either agent alone.

### 17.10 Materials and Methods for Example 17

### ELISA Binding Assay

384-well plates were coated with 1 μg/ml of target antigen diluted in PBS for 16 hrs at 4° C. After blocking for 1 hr with 1% BSA in Tris-buffered saline (TBS) at room temperature, V4-C26 hIgG4 or human IgG4 isotype control (Biolegend #403702) were serially diluted using 1% BSA made with 1×PBS at neutral pH 7 and added to the plate. For testing the binding of test article at different pH, 1% BSA was made using 1×PBS at pH of 7.5, 6.5, 6, 5.5 or 5. Post 1 hr incubation at room temperature, plates were washed three times with TBS containing 0.05% Tween 20 (TBS-T) and incubated with 1:7000 of goat anti-human IgG Fc-HRP (Abcam #ab97225) for 1 hr at room temperature. After washing, plates were developed with colorimetric detection substrate 3,3′,5,5′-tetramethylbenzidine (Turbo-TMB; Pierce) for 10 min. The reaction was stopped with 2M H2SO4, and OD was measured at 450 nm on a BioTek Synergy HT.

### Flow Cytometry and Analysis

V4-C26 hIgG4 binding to cell surface expressed VISTA on either PBMC, or HEK293T cells engineered to express VISTA, was measured by flow cytometry. Wild type HEK293T cells were transiently transfected with VISTA cDNA expression plasmids encoding human, NHP, rat, and mouse VISTA (Sinobiological) using lipofectamine 2000 (ThermoFisher Scientific #11668019) following the manufacturer's protocol. PBMCs from human, NHP, rat and mouse were procured from commercial vendor (Accegen) and blocked with Fc block (Human TruStain FcX, Biolegend #422302, Mouse TruStain Fcx, Biolegend #101320, Anti-Rat CD32, BD Pharmingen #550270 and Rhesus FcR Binding Inhibitor, ThermoFisher #14-9165-42) prior to staining. V4-C26 hIgG4 or isotype control antibodies were conjugated with APC as per manufacturers protocol.

For FACS, cells were incubated with different concentration of APC tagged V4-C26 hIgG4 or isotype control as indicated in the figure for 40 mins at 4° C. To identify myeloid cells, PBMCs were further incubated with CD45 FITC and CD11b PE. Cells were washed again and resuspended in 200 μL of FACS flow buffer (PBS with 5 mM EDTA) for flow cytometric analysis using MACSQuant 10 (Miltenyi). After acquisition, all raw data were analyzed using Flowlogic software. Cells were gated using forward and side scatter, and the percentage of positive cells was determined.

### Immunohistochemistry

Tissue microarrays (TMA) of TNBC, NSCLC, Mesothelioma and liver cancer patients (USBiomax, catalog #BR1301, #LC1401, #MS481d, #LV8013a) comprising formalin fixed paraffin embedded (FFPE) tissues were stained for VISTA (4M2-C12-mIgG2a; dilution 1:800) and VSIG3 (LS Biosciences #LS-C338858; dilution 1:300) and normal TMA (USBiomax, catalog #FDA999q) was also stained for VISTA (4M2-C12-mIgG2a). Slides were dried in a desiccator for 15 mins-1 hr and placed in EnVision™ FLEX Target Retrieval Solution, low pH (Dako, K8005/DM829) for 20 mins at 97° C. for antigen retrieval. Slides were placed in Envision Flex buffer (1×) for 10 minutes prior to transferring to the Omnis instrument for staining. Slides were stained and counterstained on the Dako Link Omnis with the Envision Flex+ detection system (kit K800) using the kit-based protocol, followed by rinsing, dehydrating and coverslipping. The TMAs were semi-quantitatively scored by light microscopy to determine the relative intensity of staining (0-3+ intensity scale), distribution and localization of VISTA and VSIG3 protein expression in normal and tumor tissues.

Cytokine Release Post V4-C26 hIgG4 Blocking of VISTA-VSIG3

PBMCs were cultured in plates coated with αCD3 monoclonal antibody (eBioscience #16-0037) and VSIG3-Fc(R&D #9229-VS) at ratios of either 1:0 (2 μg/ml αCD3 alone) and 1:2 (2 μg/ml αCD3: 4 μg/ml of VSIG3-Fc). Cells were then treated with either V4-C26 hIgG4, VSTB112 or IgG4 isotype control (Biolegend #403702) at the indicated concentration and plate was incubated at 37° C. Supernatant was harvested after 24 hrs and IFN-γ levels was measured using Human IFN-γ Uncoated ELISA kit (Invitrogen #88-7316).

### MDSC-T Cell Co-Culture

Monocytes were isolated from fresh human PBMCs via negative enrichment using Classical Monocyte Isolation Kit (Miltenyi, Germany, #130-117-337). Subsequently, monocytes were differentiated to MDSCs for 7 days in the presence of GM-CSF (10 ng/ml) (PeproTech, USA, #300-03) and IL-6 (10 ng/ml) (PeproTech, USA, #200-06). MDSCs were harvested and cultured with freshly isolated autologous PBMCs at 3:1 ratio in the presence of human anti-CD3 antibody (OKT3, 1 μg/ml) (BioLegend, USA, #317326) and in the presence or absence of test articles as indicated. Supernatant was harvested after 96 hrs and IFN-γ levels were determined via ELISA (ThermoFisher, USA, #88-7316-88).

### Neutrophil Chemotaxis Assay

Neutrophils were isolated from whole blood using MACSxpress® Whole Blood Neutrophil Isolation Kit (Miltenyi #130-104-434) and incubated with either V4-C26 hIgG4, VSTB112 or isotype control, at the indicated concentration, for 60 mins at 37° C. Post incubation, neutrophils were seeded in the upper chamber (300 μl/well) of 24 well transwell plate (Thermo Fisher #1406287) and media, with or without C5a at 50 ng/ml (Acro Biosystem #C5A-H5116), was added to the lower chamber (600 μl). Cells were incubated in the transwell for 1 hr at 37° C., after which ATP levels of the migrated neutrophils in the lower chamber was measured using CellTiter-Glo (Promega #G7571) and luminescence was measured by Victor Nova (Perkin Elmer).

### Human Allogeneic Mixed Lymphocytic Reaction (MLR)

Fresh PBMCs were isolated from human whole blood (5 donors in total) using Lymphoprep (Stemcell Technologies, #07861), following manufacture's protocol and re-suspended with CellGenix GMP DC Medium at 5×106 cells/ml. 50 μl of DC medium was added into each well of 96-well round bottom plate followed by 50 μl of PBMCs from 2 donors at 1:1 ratio in the presence of either 50 μl of V4-C26 hIgG4 or isotype control hIgG4 (InvivoGen #bgal-mab114) at 30, 10 and 1 μg/mL. A total of 10 donor pairs were used. Cells were incubated at 37° C. for 96 hrs. Supernatants were collected at 96 hrs and the supernatant cytokine levels were detected by Luminex (R&D #LXSAHM-04/07). Data was normalized to hIg4 isotype control by subtracting isotype control values from test samples.

### Animal Experiments

Balb/c mice were purchased from InVivos or Jackson Labs and CD34 engrafted humanized HiMice mice were purchased from Invivocue. All animals were housed under specific pathogen-free conditions in an AALAC accredited facility and treated in strict compliance with the Institutional Animal Care and Use Committee guidelines.

### In Vivo Tumor Growth Assays

Mice were subcutaneously implanted with tumor cells (105 for CT26, 106 for HCT15 and 5×10⁶ for A549) in the right flank or for orthotopic breast model, tumor cells were implanted into the mammary fat pad (2×10⁴ 4T1 cells). 3-6 days post implantation, mice were treated twice a week with the indicated dose and interval of the test articles. Treatment was administered intraperitoneally for all subcutaneous models, and intratumorally for the orthotopic model. Tumor volume was measured using calipers as described in Thakkar et al., Mol Cancer Ther (2020) 19:490-501).

### Statistical Analysis

Statistical analysis was performed using GraphPad Prism. Data acquired with two variables (dose titrations) was analyzed with 2-way ANOVAs followed by Tukey's multiple comparisons test. For comparisons between two groups, an unpaired t-test was performed. Values of +p≤0.05, ** p≤0.01, *** p≤0.001, **** p≤0.0001, were considered significant.

### Cross-Species Antibody Binding Affinity Measurement

The affinity of V4-C26 hIgG4 was determined by Surface Plasmon Resonance (SPR) using a Biacore. The assay was performed using a CM5 sensor chip (Cytiva #29104988). A Biacore HIS capture kit (GE Healthcare #28-9950-56) was employed to immobilize human, NHP, rat and mouse VISTA-HIS on-chip surface or left alone for background signal correction. Briefly, VISTA-HIS was captured for a contact time of 60 s at a flow rate of 5 μl/min. V4-C26 hIgG4 was flowed in two-fold serial dilutions from 50E-9 M to 390E⁻¹² M for human, NHP and rat VISTA and from 12.5E-9 M to 390E-12 M for mouse VISTA, at a flow rate of 30 μl/min for 90 sec association and 3000 sec dissociation at RT. The obtained sensograms were analyzed using Biacore T200 software and KD was calculated by fitting to a 1:1 binding kinetics model.

### Antibody-Dependent Cellular Cytotoxicity and Complement-Dependent Cytotoxicity

96 well plates were coated with either 1 μg/well of human C1q protein or 0.5 μg/well of human CD16a in PBS at 4° C. Post overnight incubation, plates were washed thrice and blocked for 1 hr with blocking buffer at room temperature. Plates were incubated with V4-C26 hIgG4 or isotype control for 1 hr at room temperature and incubated with 1:7000 dilution of HRP-conjugated anti-human Fc antibody for 1 hr at room temperature. Colorimetric reactions were developed using standard protocol as described for ELISA above.

sc-RNA-seq

To determine the cell types in which VISTA transcripts were most highly expressed, a publicly available 68k PBMC dataset was retrieved from the 10× Genomics website (Stuart et al., Biorxiv (2018), 460147) and processed using Seurat v3.2 (Stuart et al., Cell (2019) 177:1888-1902.e21). Cells with more than 6% mitochondrial transcripts, or less than 200 distinct transcripts were excluded. Data was normalized using default parameters and variable features identified using the VST method with 20,000 features. Data was subsequently scaled and then a PCA decomposition run with 15 principal components. A UMAP projection was carried out using default parameters and the FindClusters function run with a resolution of 0.8. To label the clusters the PBMC3k dataset (Hafemeister and Satija, Biorxiv (2019), 576827) from the SeuratData package was used as a reference to map cell identities.

### Bulk RNA-seq

10 samples per condition (V4-C26, V9, anti-PD1, and IgG4) were taken from the MLR assay 96 hours after treatment. These were prepared for RNA-seq using the NEBNext Ultra II RNA library preparation kit (NEB #E7770S) and run on an Illumina NovaSeq S4 flowcell with v1.5 chemistry. Adaptor-trimmed and filtered data was evaluated using FASTQC and MultiQC (Ewels et al., Bioinformatics (2016) 32:3047-3048) and reads aligned to the GRC37 human reference transcriptome using Kallisto v0.46 (Bray et al., Nat Biotechnol (2016) 34:525-527). Data was imported to R using TxImport (Soneson et al., F1000research (2015) 4:1521). Normalized gene counts and gene-level differential expression was obtained using DESeq2 (Love et al., Genome Biol (2014) 15:550). Pathway activity was estimated using the SPEED2 (Rydenfelt et al., Nucleic Acids Res (2020) 48: W307-W312) package using default parameters.

### CT26 Tumor Profiling

Single cell suspensions were generated by digestion of finely cut tumor tissue with 0.1 mg/ml of DNase I (Sigma, USA, #11284932001) and 1 mg/ml Collagenase (Sigma, USA, #1108866001) for 45 minutes at 37° C. Fc receptors were blocked with Human TruStain FcX (BioLegend, USA, #422302). Cells were stained with fluorophore conjugated antibodies for markers of immune cells (Table S4), washed and resuspended in buffer (1×PBS+0.5% BSA+1 mM EDTA). Frequency of immune cell populations was determined via flow cytometry. Data was acquired on MACSQuant 10 (Milteny Biotec, Germany, #130-096-343) and analyzed using FlowLogic software V7.

### CT26 Antigen Recall Assay

Single cell tumor suspensions were generated as described earlier. TILs (CD45+) or T cells (CD4+/CD8+) were enriched from the cell suspension using CD45 MicroBeads (Milteny Biotec, Germany, #130-110-618) or CD4/CD8 MicroBeads (Milteny Biotec, Germany, #130-116-480) as per manufacturer's protocol. CT26 cells (T: target cells), seeded 24 hrs prior, were co-cultured with enriched TILs or T cells (E: effector cells) from tumors for 72 hours at effector to target (E: T) cell ratios as indicated. All conditions were in triplicates. Cell viability was determined by CellTiter Glo (Promega, USA, #G7571). IFN-γ levels in supernatants were determined by ELISA (Invitrogen, USA, #BMS606). Lysis was calculated as: % Lysis=((T−E:T)/T)*100.

### Pharmacokinetics

A single dose pharmacokinetic profile of V4-C26 hIgG4 was evaluated in male and female Balb/c mice,

Sprague Dawley rats and cynomolgus monkeys. V4-C26 hIgG4 was administered in a single dose at the indicated concentration via intraperitoneal injection in mice, IV bolus infusion into the tail vein in rats, and IV bolus infusion into the peripheral vein in monkeys. Blood was drawn at different timepoints post dosing and antibody concentration in the serum was quantified by ELISA. The parameters for the pharmacokinetic analysis were derived from a non-compartmental model: maximum concentration (Cmax), AUC (0-336 hr), AUC (0-infinity), half-life (t1/2), clearance (CL) and volume of distribution at steady state (Vss).

### Example 18: Antibody Formulation Development

Formulation development was performed for an antigen-binding molecule comprising V4-C26 variable regions, human IgG4 heavy chain constant regions and kappa light chain, and which is comprised of the heavy chain of SEQ ID NO:331 and the light chain of SEQ ID NO:317. The antigen-binding molecule is produced by a cell of the cell line deposited 7 May 2021 as ATCC Patent Deposit Number PTA-127063. This antigen-binding molecule may be referred to as ‘HMDB-002’ in this Example.

### 18.1 Formulations

The product stability of HMDB-002 was evaluated in different liquid formulations. The formulation development includes 1-month stability at high temperature and 2-8° C., freeze-thaw stress study and high concentration stress study. The analytical testing was designed identify the formulations that display the best molecular stability with respects to size variants, charge variants and binding potency.

Purified material (MabSelect SuRe LX (LX; 3L bioreactor run 1), further purified by Superdex and Capto Q) was dialysed against different formulation buffers using Slide-A-Lyzer, 20k MWCO (Cat #66030, Thermo Scientific). The samples were first over-concentrated beyond target concentration (approx. 1-1.25 folds) then adjusted back to target concentration by diluting with formulation buffer. The processed samples were filtered through 0.1 um sterile filters (Cat #16553, Sartorius) and dispensed in a biosafety cabinet into a 1.8 mL cryopreservation vial (Cat #NNC #368632, Thermo Scientific), with 1 mL filling volume.

The formulation buffers tested had the following compositions:

| Formulation | | Buffer System | Sucrose | NaCl | Polysorbate | Polysorbate |
|---|---|---|---|---|---|---|
| ID | pH | (20 mM) | (%, w/v) | (mM) | 20 (%) | 80 (%) |
| F1 | 5.5 | Histidine | 8 | 0 | 0 | 0.02 |
| F2 | 5.8 | Histidine | 8 | 0 | 0 | 0.02 |
| F3 | 5.8 | Histidine | 4 | 0 | 0 | 0.02 |
| F4 | 5.8 | Histidine | 2 | 0 | 0 | 0.02 |
| F5 | 6.3 | Histidine | 8 | 0 | 0 | 0.02 |
| F6 | 5.8 | Histidine | 8 | 0 | 0.02 | 0 |
| F7 | 5.5 | Sodium | 8 | 0 | 0 | 0.02 |
| | | Acetate | | | | |
| F8 | 5.5 | Sodium | 8 | 0 | 0 | 0.02 |
| | | Succinate | | | | |
| F9 | 5.8 | Histidine | 0 | 0 | 0 | 0.02 |
| F10 | 5.8 | Histidine | 0 | 150 | 0 | 0 |

### 18.2 Stressed Conditions

The ten formulations were subjected to high temperature, high concentration and freeze-thawing stresses. In total, 4 vials (including two vial sizes: 4 mL/vial for high concentration; 1 mL/vial for the remaining conditions) were prepared for each formulation. 10 formulations with 1 concentration (10 mg/mL), 3 stress conditions with 1 control group at 2-8° C. were tested, among which stability testing over a defined time period were conducted for storage conditions 40° C. and 2-8° C.

Formulation stress conditions:
Freeze-thawing: Six cycles of freezing at −80° C. and thawing at room temperature were performed. Between each cycle, the vials were kept at −80° C. for deep frozen until thawed. Each vial was approximately 5 cm apart from each other during freezing or thawing. A small aliquot of approx. 80 μL was taken from the vial and stored at 2-8° C. until analysis, the vials were returned to −80° C. after sampling for 10 formulations are done.
High temperature: one vial from each formulation was stored in (Infors HT Multitron) incubator with temperature setting point of 40° C., at predetermined time point. An approximate 150 μL was taken inside the biosafety cabinet to prevent contamination. The aliquoted samples were then stored at 2-8° C. until analysis, the remaining vials were returned to incubator.
High concentration: Samples were concentrated with 20K Da MWCO Amicon protein concentrator (Cat #UFC905096, Millipore) in a bench top centrifuge at 4000 g, 4° C. The concentration was monitored every 20 mins, until each targeted concentration was exceeded. Sample concentration was then adjusted back to its nearest target concentration.

The aliquoted samples were analyzed for precipitation by visual inspection, pH by pH meter with microelectrode, concentration by Nanodrop Lite 2000, aggregation by HLPC-SEC, charge variance by HPLC-CEX, potency by antigen binding ELISA and osmolality by Nova Flex. The stress conditions and analytical assays are shown below.

| Stress | Visual | | | HPLC- | HPLC- | Antigen | |
|---|---|---|---|---|---|---|---|
| condition | Inspection | pH | Concentration | SEC | CEX | Binding | Osmolality |
| 37-43° C. | X | X | X | X | X | X | X |
| 2-8° C. | X | X | X | X | X | X | X |
| High | X | | X | X | | | |
| Concentration | | | | | | | |
| Freeze-Thaw | | | | X | | | |

### 18.3 Stability Studies

The stability of HMBD-002 at 10 mg/mL in the 10 formulations was evaluated for a total period of one month at 40° C.±3° C. and 6-month at 5° C.±3° C., respectively. The samples were analyzed at t=0 upon release from downstream process. At time points day 10, 20, 30, 60, 90, 180, the samples were analyzed using the assays described herein.

### 18.4 Analytical Techniques.

Protein content by A280: Determination of protein content by the measurement of UV light absorbance was based on the specific characteristics of abundance of tryptophan, tyrosine and cysteine present in the protein. The theoretical extinction coefficient for HMBD-002 is 1.64 mL·mg-1·cm-1.
Visual inspection: The sample was inspected for visible particles and precipitation.
Purity and aggregate content by HPLC-SEC: The purity of the antibody was measured with High Performance Liquid Chromatography, based on HMBD-002 SEC SOP v1.0 with major modification of changing analytical column to XBridge BEH200 SEC 3.5μ 7.8×300 mm column (Cat #176003596, Waters).
Charge heterogeneity by HPLC-CEX: The charged distribution of the antibody was performed by ion-exchange on a High-Performance Liquid Chromatography system, based on HMBD-002 CEX SOP v1.0.
pH: The pH measurement was performed after a 3-point calibration of the pH meter with commercially available standard solutions at pH 7.0, pH 4.0 and pH 10.0.
Osmolality: Osmolality was measured by Nova Flex and is determined using freeze point depression method.
Binding ELISA: HMBD-002-V4C26 ELISA (Antigen Binding) SOP v1.0 is used.

### 18.5 Results

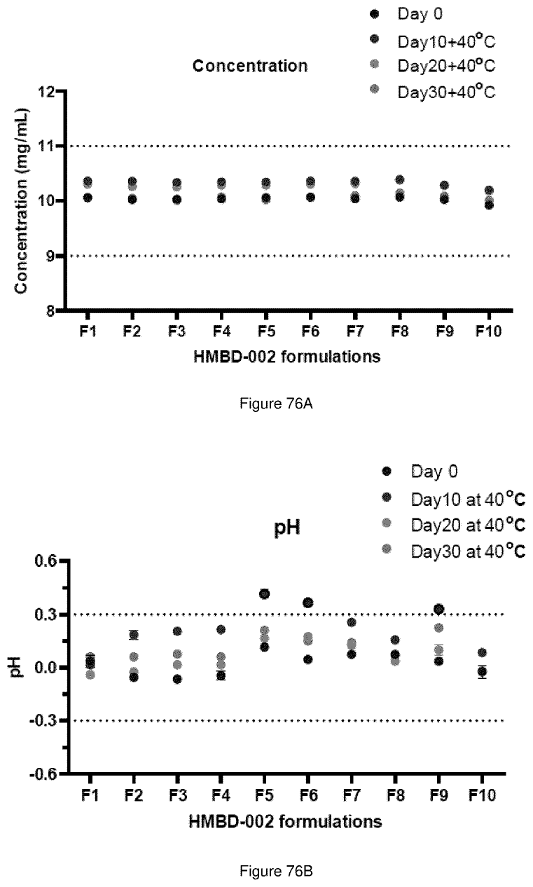

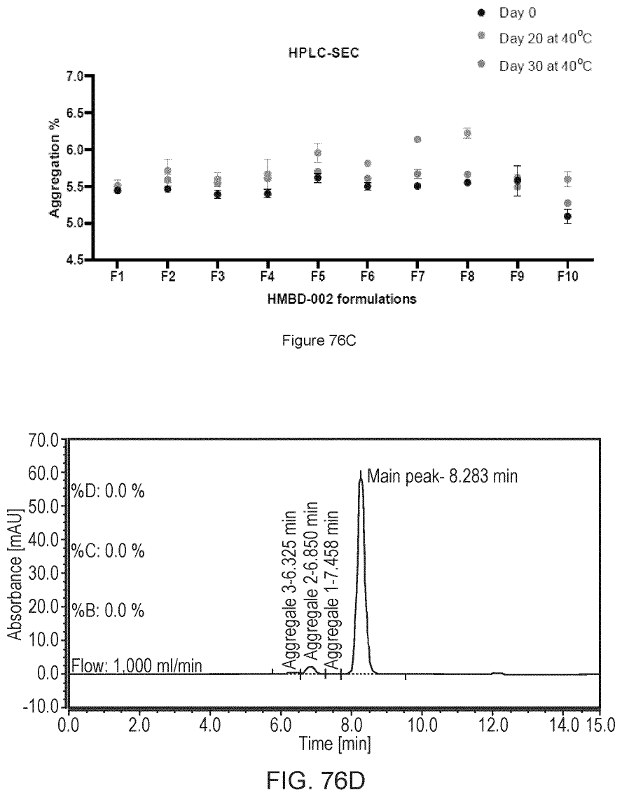

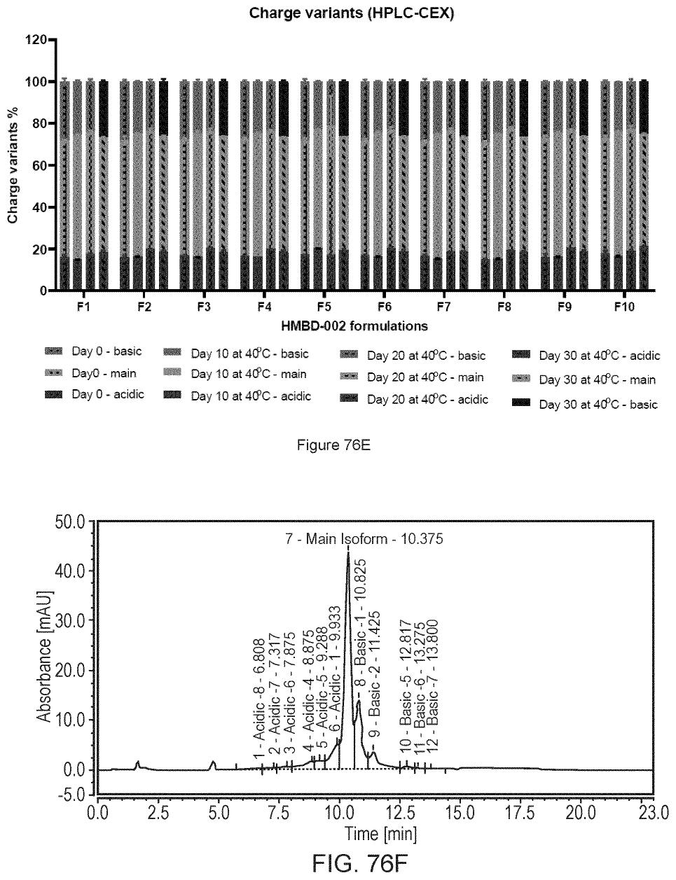

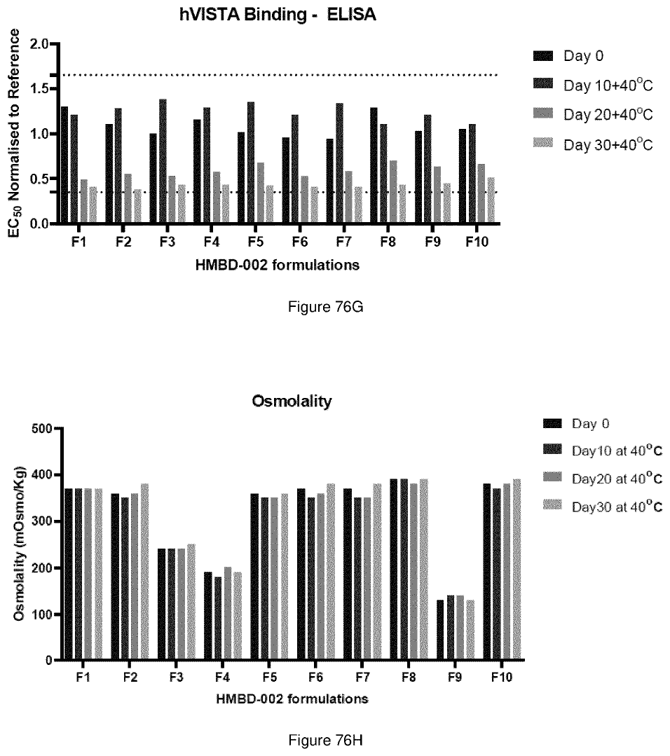

The results from the high temperature stability study are shown in FIGS. 76A to 76?.

FIG. 76A shows that protein concentrations were found to be stable for 1 month at 40° C.±3° C., for all formulations.

FIG. 76B shows that pH fluctuation was found to be less than 0.3, which is generally acceptable during long term storage, for all formulations. The 3 measurement points at day 10 for formulation F5, F6 and F9 (highlighted by black circle) are considered as outliners since measurements taken from day 20 and day 30 are all showing much smaller variations towards to target pH.

FIG. 76C shows HPLC-SEC aggregation. The protein samples had approximately 5.5% aggregation before putting into 40° C. chamber. Although the aggregation measurements at day 20 were higher than that of day 30, there's a clear trend that F5-F8 and F10 were more susceptible to form aggregates during long term storage at high temperature compared to other formulations. FIG. 76D shows representative results of HMBD-002 in formulation F1 at Day 30 in 40° C.

FIG. 76E shows that acidic variants generally showed slightly increasing trend with main isoform fluctuating at range between 54%-60%, as measured by HPLC-CEX. As the antibody sequence shows low liability to deamidation, isomerization, glycation, racemization, succinimide formation, undesired glycosylation, it is probable that the chemical stability of drug substance is preserved in all formulations. The aggregation peaks, which normally correspond to part of basic-1 peak for HMBD-002 drug substance, did not show further increase confirming that common factors in these 10 formulations contributed to prevent aggregation formation (as observed in the HPLC-SEC data).

FIG. 76F shows representative HPLC-CEX results of HMBD-002 in formulation F1 at Day 30 in 40° C., The six acidic peaks, single main isoform peak, and five basic peaks are well dispersed in the analysis window of approx. 8 mins which indicates that the method is sensitive enough to demonstrate stability.

FIG. 76G shows that there was an increase in the affinity of HMBD-002 to its human target (VISTA) in all formulations from day 20 at 40° C. On day 30, the affinity is further increased to a maximum 1.65-fold (F2). EC₅₀ data from antigen binding ELISA studies are provided below.

| | EC₅₀ concentration (M) | | | |
|---|---|---|---|---|
| | | Day 10 + | Day 20 + | Day 30 + |
| Formulations | Day 0 | 40° C. | 40° C. | 40° C. |
| F1 | 4.00E−11 | 3.06E−11 | 1.72E−11 | 2.15E−11 |
| F2 | 3.39E−11 | 3.22E−11 | 1.97E−11 | 2.02E−11 |
| F3 | 3.08E−11 | 3.48E−11 | 1.89E−11 | 2.27E−11 |
| F4 | 3.57E−11 | 3.25E−11 | 2.05E−11 | 2.27E−11 |
| F5 | 3.10E−11 | 3.41E−11 | 2.39E−11 | 2.23E−11 |
| F6 | 2.94E−11 | 3.06E−11 | 1.86E−11 | 2.18E−11 |
| F7 | 2.89E−11 | 3.39E−11 | 2.06E−11 | 2.17E−11 |
| F8 | 3.95E−11 | 2.80E−11 | 2.48E−11 | 2.27E−11 |
| F9 | 3.15E−11 | 3.05E−11 | 2.23E−11 | 2.37E−11 |
| F10 | 3.21E−11 | 2.81E−11 | 2.35E−11 | 2.70E−11 |
| Reference | 3.07E−11 | 2.53E−11 | 3.55E−11 | 5.27E−11 |

FIG. 76H shows that all formulations showed minimal change in osmolality for 1-month storage at 40° C.

When HMBD-002 was formulated at high concentrations, visual inspection revealed that HMBD-002 showed good solubility in 8 formulations up to 200 mg/ml concentration, shown below. However, formulations F5 and F9 did not support good solubility beyond 100 mg/mL.

| | Concentration (mg/mL) | | | | |
|---|---|---|---|---|---|
| Formulations | 10 | 50 | 100 | 200 | Remarks |
| F1 | 9.88 | 50.00 | 107.89 | 214.40 | Transparent, clear |
| F2 | 10.07 | 50.74 | 116.95 | 213.37 | Transparent, clear |
| F3 | 9.94 | 50.23 | 111.11 | 205.41 | Transparent, clear |
| F4 | 10.34 | 52.83 | 116.33 | 211.05 | Transparent, clear |
| F5 | 9.88 | 50.74 | 136.96 | 201.08 | Very cloudy and viscous |
| F6 | 9.89 | 51.18 | 106.36 | 215.51 | Transparent, clear |
| F7 | 9.95 | 50.16 | 118.36 | 209.02 | Transparent, clear |
| F8 | 10.01 | 50.78 | 108.42 | 208.25 | Transparent, clear |
| F9 | 10.08 | 50.77 | 102.15 | 201.96 | Relatively cloudy and |
| | | | | | viscous |
| F10 | 9.81 | 50.29 | 100.25 | 158.60 | Transparent, clear |

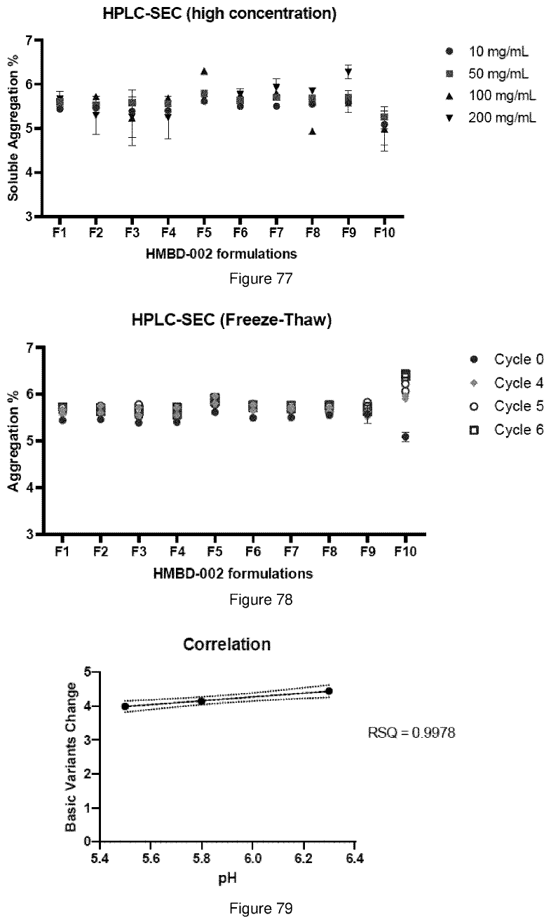

FIG. 77 shows that formulations F1, F2, F3, F4 and F10 showed good potential in formulating to high concentration with minimum change in soluble aggregation (measured by HPLC-SEC). For F5 and F9, insoluble aggregations were removed by centrifugation and further filtered by 0.22 μm filter, only soluble portions were analysed for HPLC-SEC.

The HMBD-002 formulations subjected to freeze-thaw stress were analysed by HPLC-SEC for aggregation propensity. FIG. 78 shows that HMBD-002 showed good stability in 9 formulations after up to 6 cycles of freezing and thawing. Formulation F10 showed aggregation propensity after freeze-thaw.

The table below summarises the key results from the stability analysis. Pale grey results indicates favourable protein stability, while mid grey results indicates unfavourable protein stability.

| | | | | | | | | | | hVISTA |
|---|---|---|---|---|---|---|---|---|---|---|
| | | pH | | Aggregation | Acidic Variants | | Basic Variants | | | ELISA |
| Formulation | Concentration | | Day | % | | Day | | Day | Highest stable | (EC₅₀)¹ at |
| ID | (Day 30) | Baseline | 30 | (Day 30) | Baseline | 30 | Baseline | 30 | concentration | Day 30 |
| F1 | 10.06 | 5.5 | 5.56 | 5.505 | 15.87 | 18.42 | 28.06 | 26.63 | 214.40 | 2.15E−11 |
| F2 | 10.05 | 5.8 | 5.86 | 5.585 | 15.97 | 18.31 | 28.05 | 26.26 | 213.37 | 2.02E−11 |
| F3 | 10.01 | 5.8 | 5.88 | 5.545 | 16.96 | 18.57 | 27.61 | 26.33 | 205.41 | 2.27E−11 |
| F4 | 10.07 | 5.8 | 5.86 | 5.605 | 16.69 | 18.65 | 27.38 | 26.50 | 211.05 | 2.27E−11 |
| F5 | 10.02 | 6.3 | 6.51 | 5.695 | 17.03 | 19.34 | 28.22 | 26.09 | 136.96 | 2.23E−11 |
| F6 | 10.06 | 5.8 | 5.95 | 5.605 | 16.85 | 18.87 | 28.41 | 26.19 | 215.51 | 2.18E−11 |
| F7 | 10.10 | 5.5 | 5.64 | 5.665 | 16.43 | 18.65 | 28.50 | 26.52 | 209.02 | 2.17E−11 |
| F8 | 10.14 | 5.5 | 5.57 | 5.660 | 14.93 | 18.60 | 29.21 | 26.54 | 208.25 | 2.27E−11 |
| F9 | 10.08 | 5.8 | 6.03 | 5.490 | 16.05 | 18.84 | 27.21 | 26.25 | 102.15 | 2.37E−11 |
| F10 | 10.01 | 5.8 | 5.78 | 5.270 | 17.67 | 21.11 | 26.95 | 25.06 | 158.60 | 2.70E−11 |

¹Reference standard: 5.27E−11 M

The table below shows the evaluation of pH in charge variant profile.

| | | | | | | | | Total |
|---|---|---|---|---|---|---|---|---|
| | | Acidic | Acidic | | Basic | Basic | | Change |
| | Comparison | Variants | Variants | Delta | Variants | Variants | Delta | (Absolute |
| Evaluation | Group | (D 0) | (D 30) | (Acidic) | (D 0) | (D 30) | (Basic) | Values) |
| pH | F1 (5.5) | 15.865 | 18.42 | 2.55 | 28.06 | 26.63 | −1.44 | 3.99 |
| | F2 (5.8) | 15.965 | 18.31 | 2.34 | 28.05 | 26.26 | −1.80 | 4.14 |
| | F5 (6.3) | 17.030 | 19.34 | 2.31 | 28.22 | 26.09 | −2.13 | 4.44 |

FIG. 79 shows the correlation of pH and Total Charge Variance Change for formulations F1, F2, and F5.

### 18.6 Conclusions

Formulations F1, F2, F3, and F4 showed superior performance over other formulation candidates in preserving the molecular stability with respects to key quality attributes during manufacturing. Furthermore, evaluation on pH change versus charge variants profile change during long term storage at 40° C. indicates a good correlation between pH decrease and charge variants absolute values decrease.

Overall, formulation F1 (20 mM Histidine+8% sucrose+0.02% polysorbate 80, pH 5.5) showed the best in preserving the antibody's integrity and stability at all conditions tested and is selected as the lead formulation for clinical material. In addition, considering the relatively high sucrose level (8% w/v) and protein stability in the high concentration study, the final drug substance obtained for clinical material should be formulated to 50 mg/mL.

### 18.7 Stability of Drug Substance

HMBD-002 clinical drug substance, formulated at 50 mg/mL in formulation F1 above, was assessed for its stability at long-term storage condition (−80° C.), accelerated storage condition (5° C.), stress storage condition (25° C.), and under 6 cycles of freeze/thaw conditions. Results are provided below. HMBD-002 Drug Substance at −80° C.:

| | | Acceptance | Timepoint | | |
|---|---|---|---|---|---|
| Attribute | Assay | Criteria | T0¹ | 1 M | 3 M |
| Color | Visual Method | ≤Y3 | <Y6² | <Y6 | <Y6 |
| General | pH | 5.0 to 6.0 | 5.7 | 5.6 | 5.6 |
| Content | Protein Content | 42.5 to 57.5 mg/mL | 52.7 mg/mL | 53.5 mg/mL | 54.5 mg/mL |
| Potency | Binding assay | 60% to 140% | 92% relative potency | 101% relative potency | 90% relative potency |
| | | relative potency | | | |
| Purity | SE-HPLC³ | % Main peak: ≥92% | % Main peak: 95% | % Main peak: 95% | % Main peak: 95% |
| | CE-SDS⁴ | % LC + HC⁵: ≥95% | % LC + HC⁵: 98% | % LC + HC⁵: 98% | % LC + HC⁵: 99% |
| | (Reduced) | | | | |
| | CE-SDS⁴ (Non- | % Main peak: ≥90% | % Main peak: 96% | % Main peak: 95% | % Main peak: 95% |
| | Reduced) | | | | |
| | CEX-HPLC⁶ | % Main peak: | % Main peak: 58% | % Main peak: 57% | % Main peak: 59% |
| | | Report results | | | |
| | | % Acidic Peaks: | % Acidic peaks: 18% | % Acidic peaks: 17% | % Acidic peaks: 17% |
| | | Report results | | | |
| | | % Basic Peaks: | % Basic peaks: 24% | % Basic peaks: 26% | % Basic peaks: 24% |
| | | Report results | | | |

¹Unless specified the Time 0 (T0) refers to the batch release data
²Data from 1 M at −80° C.
³Size Exclusion - High Pressure Liquid Chromatography
⁴Capillary Electrophoresis - Sodium Dodecyl Sulfate
⁵Light chain + Heavy chain
⁶Cation Exchange - High Pressure Liquid Chromatography

HMBD-002 Drug Substance at 5° C.:

| | | Acceptance | Timepoint | | |
|---|---|---|---|---|---|
| Attribute | Assay | Criteria | T0¹ | 1 M | 3 M |
| Color | Visual Method | ≤Y3 | <Y6² | <Y6 | <Y6 |
| General | pH | 5.0 to 6.0 | 5.7 | 5.7 | 5.6 |
| Content | Protein Content | 42.5 to 57.5 mg/mL | 52.7 mg/mL | 54.5 mg/mL | 53.9 mg/mL |
| Potency | Binding assay | 60% to 140% | 92% relative potency | 105% relative potency | 89% relative potency |
| | | relative potency | | | |
| Purity | SE-HPLC³ | % Main peak: ≥92% | % Main peak: 95% | % Main peak: 95% | % Main peak: 95% |
| | CE-SDS⁴ | % LC + HC⁵: ≥95% | % LC + HC⁵: 98% | % LC + HC⁵: 98% | % LC + HC⁵: 99% |
| | (Reduced) | | | | |
| | CE-SDS⁴ (Non- | % Main peak: ≥90% | % Main peak: 96% | % Main peak: 95% | % Main peak: 95% |
| | Reduced) | | | | |
| | CEX-HPLC⁶ | % Main peak: | % Main peak: 58% | % Main peak: 58% | % Main peak: 59% |
| | | Report results | | | |
| | | % Acidic Peaks: | % Acidic peaks: 18% | % Acidic peaks: 18% | % Acidic peaks: 17% |
| | | Report results | | | |
| | | % Basic Peaks: | % Basic peaks: 24% | % Basic peaks: 25% | % Basic peaks: 24% |
| | | Report results | | | |

¹Unless specified the Time 0 (T0) refers to the batch release data
²Data from 1 M at −80° C.
³Size Exclusion - High Pressure Liquid Chromatography
⁴Capillary Electrophoresis - Sodium Dodecyl Sulfate
⁵Light chain + Heavy chain
⁶Cation Exchange - High Pressure Liquid Chromatography

HMBD-002 Drug Substance at 25° C., 60% relative humidity:

| | | | Timepoint | | | |
|---|---|---|---|---|---|---|
| Attribute | Assay | Acceptance Criteria | T0¹ | 1 D | 3 D | 7 D |
| Color | Visual Method | ≤Y3 | <Y6² | <Y6 | <Y6 | <Y6 |
| General | pH | 5.0 to 6.0 | 5.7 | 5.7 | 5.7 | 5.7 |
| Content | Protein Content | 42.5 to 57.5 mg/mL | 52.7 mg/mL | 52.0 mg/mL | 54.5 mg/mL | 53.3 mg/mL |
| Potency | Binding assay | 60% to 140% relative | 92% relative | 87% relative | 94% relative | 90% relative |
| | | potency | potency | potency | potency | potency |
| Purity | SE-HPLC³ | % Main peak: ≥92% | % Main peak: | % Main peak: | % Main peak: | % Main peak: |
| | | | 95% | 95% | 95% | 95% |
| | CE-SDS⁴ (Reduced) | % LC + HC⁵: ≥95% | % LC + HC⁵: | % LC + HC⁵: | % LC + HC⁵: | % LC + HC⁵: |
| | | | 98% | 99% | 98% | 98% |
| | CE-SDS⁴ (Non- | % Main peak: ≥90% | % Main peak: | % Main peak: | % Main peak: | % Main peak: |
| | Reduced) | | 96% | 95% | 95% | 95% |
| | CEX-HPLC² | % Main peak: | % Main peak: | % Main peak: | % Main peak: | % Main peak: |
| | | Report results | 58% | 57% | 58% | 59% |
| | | % Acidic Peaks: | % Acidic | % Acidic | % Acidic | % Acidic |
| | | Report results | peaks: 18% | peaks: 18% | peaks: 17% | peaks: 17% |
| | | % Basic Peaks: | % Basic peaks: | % Basic peaks: | % Basic peaks: | % Basic peaks: |
| | | Report results | 24% | 25% | 25% | 24% |

¹Unless specified the Time 0 (T0) refers to the batch release data
²Data from 1 M at −80° C.
³Size Exclusion -High Pressure Liquid Chromatography
⁴Capillary Electrophoresis - Sodium Dodecyl Sulfate
⁵Light chain + Heavy chain
⁶ Cation Exchange - High Pressure Liquid Chromatography

HMBD-002 Drug Substance subjected to freeze/thaw conditions:

| | | | | Freeze and Thaw |
|---|---|---|---|---|
| Attribute | Assay | Acceptance Criteria | T0¹ | 6 cycles −80° C. to 25° C. |
| Color | Visual Method | ≤Y3 | <Y6 | <Y6 |
| General | pH | 5.0 to 6.0 | 5.7 | 5.7 |
| Content | Protein Content | 42.5 to 57.5 mg/mL | 52.7 mg/mL | 54.1 mg/mL |
| Potency | Binding assay | 60% to 140% relative | 92% relative potency | 79% relative potency |
| | | potency | | |
| Purity | SE-HPLC² | % Main peak: ≥92% | % Main peak: 95% | % Main peak: 95% |
| | CE-SDS³ (Reduced) | % LC + HC⁴: ≥95% | % LC + HC⁴: 98% | % LC + HC⁴: 98% |
| | CE-SDS³ (Non- | % Main peak: ≥90% | % Main peak: 96% | % Main peak: 95% |
| | Reduced) | | | |
| | CEX-HPLC⁵ | % Main peak: | % Main peak: 58% | % Main peak: 59% |
| | | Report results | | |
| | | % Acidic Peaks: | % Acidic peaks: 18% | % Acidic peaks: 17% |
| | | Report results | | |
| | | % Basic Peaks: | % Basic peaks: 24% | % Basic peaks: 24% |
| | | Report results | | |

¹Unless specified the Time 0 (T0) refers to the batch release data
²Size Exclusion - High Pressure Liquid Chromatography
³Capillary Electrophoresis - Sodium Dodecyl Sulfate
⁴Light chain + Heavy chain
⁵Cation Exchange - High Pressure Liquid Chromatography

The results generated demonstrate that the drug substance remains stable at the recommended storage conditions up to 6 months. Based on the stability results the assigned expiry period will be 12 months.

### 18.8 Stability of Drug Product

HMBD-002 clinical drug product, formulated at 50 mg/mL in formulation F1 above, was assessed for its stability at long-term storage condition (−20° C.), accelerated storage condition (5° C.), stress storage conditions (25° C. and 40° C.), and under 6 cycles of freeze/thaw conditions. Results are provided below.

HMBD-002 Drug Product at −20° C.:

| | | Acceptance | Timepoints | | |
|---|---|---|---|---|---|
| Attribute | Assay | Criteria | T0¹ | 1 M | 3 M |
| Appearance | Visual method | Liquid, practically | Liquid, practically | Liquid, practically | Liquid, practically |
| | | free from particles | free from particles | free from particles | free from particles |
| Color | Visual method | ≤Y3 | <Y6 | <Y6 | <Y6 |
| Clarity | Turbidimetry method | ≤Ref. IV | <Ref. III | <Ref. III | <Ref. III |
| General | pH | 5.0 to 6.0 | 5.6 | 5.7 | 5.8 |
| | Liquid particulate | ≥10 μm: ≤6000/container | ≥10 μm: | ≥10 μm: | ≥10 μm: |
| | matter | | 25 per container | 22 per container | 11 per container |
| | | ≥25 μm: ≤600/container | ≥25 μm: | ≥25 μm: | ≥25 μm: |
| | | | 1 per container | 0 per container | 0 per container |
| Content | Protein content | 42.5 to 57.5 mg/mL | 54.5 mg/mL | 51.9 mg/mL | 52.4 mg/mL |
| Potency | Binding assay | 60% to 140% | 84% relative | 102% relative | 95% relative |
| | | relative potency | potency | potency | potency |
| Purity | SE-HPLC² | % Main peak: ≥92% | % Main peak: 95% | % Main peak: 95% | % Main peak: 95% |
| | CE-SDS³ (Reduced) | % LC + HC⁴ ≥ 95% | % LC + HC⁴: 98% | % LC + HC⁴: 98% | % LC + HC⁴: 99% |
| | CE-SDS³ (Non- | % Main peak: ≥90% | % Main peak: 95% | % Main peak: 95% | % Main peak: 96% |
| | Reduced) | | | | |
| | CEX-HPLC⁵ | % Main peak: | % Main peak: 58% | % Main peak: 57% | % Main peak: 58% |
| | | Report results | | | |
| | | % Acidic peaks: | % Acidic peaks: 17% | % Acidic peaks: 17% | % Acidic peaks: 17% |
| | | Report results | | | |
| | | % Basic peaks: | % Basic peaks: 25% | % Basic peaks: 25% | % Basic peaks: 24% |
| | | Report results | | | |
| Safety | Sterility/Container | Sterility: Sterile | Sterility: Sterile | Not tested | Not tested |
| | Closure Integrity | CCI: Pass⁶ | | | |
| | (CCI) | | | | |

¹Unless specified, the Time 0 (T0) refers to the batch release data
²Size Exclusion - High Pressure Liquid Chromatography
³Capillary Electrophoresis - Sodium Dodecyl Sulfate
⁴Light chain + Heavy chain
⁵Cation Exchange - High Pressure Liquid Chromatography
⁶Pass indicates that the sample showed no dye ingress

HMBD-002 Drug Product at 5° C., upright orientation:

| | | | Timepoints | | | |
|---|---|---|---|---|---|---|
| Attribute | Assay | Acceptance Criteria | T0¹ | 14 D | 1 M | 3 M |
| Appearance | Visual method | Liquid, practically | Liquid, practically | Liquid, practically | Liquid, practically | Liquid, practically |
| | | free from particles | free from particles | free from particles | free from particles | free from particles |
| Color | Visual method | ≤Y3 | <Y6 | <Y6 | <Y6 | <Y6 |
| Clarity | Turbidimetry | ≤Ref. IV | <Ref. III | <Ref. III | <Ref. III | <Ref. III |
| | method | | | | | |
| General | pH | 5.0 to 6.0 | 5.6 | 5.6 | 5.6 | 5.8 |
| | Liquid | ≥10 μm: ≤6000/ | ≥10 μm: | ≥10 μm: | ≥10 μm: | ≥10 μm: |
| | particulate | container | 25 per container | 13 per container | 28 per container | 20 per container |
| | matter | ≥25 μm: ≤600/ | ≥25 μm: | ≥25 μm: | ≥25 μm: | ≥25 μm: |
| | | container | 1 per container | 0 per container | 1 per container | 0 per container |
| Content | Protein content | 42.5 to 57.5 | 54.5 mg/mL | 53.8 mg/mL | 52.0 mg/mL | 51.7 mg/mL |
| | | mg/mL | | | | |
| Potency | Binding assay | 60% to 140% | 84% relative | 93% relative | 103% relative | 98% relative |
| | | relative potency | potency | potency | potency | potency |
| Purity | SE-HPLC² | % Main peak: ≥92% | % Main peak: | % Main peak: | % Main peak: | % Main peak: |
| | | | 95% | 95% | 95% | 95% |
| | CE-SDS³ | % LC + HC⁴ ≥95% | % LC + HC⁴: 98% | % LC + HC⁴: 98% | % LC + HC⁴: 98% | % LC + HC⁴: 98% |
| | (Reduced) | | | | | |
| | CE-SDS³ (Non- | % Main peak: ≥90% | % Main peak: | % Main peak: | % Main peak: | % Main peak: |
| | Reduced) | | 95% | 95% | 95% | 96% |
| | CEX-HPLC⁵ | % Main peak: | % Main peak: | % Main peak: | % Main peak: | % Main peak: |
| | | Report results | 58% | 57% | 57% | 58% |
| | | % Acidic Peaks: | % Acidic peaks: | % Acidic peaks: | % Acidic peaks: | % Acidic peaks: |
| | | Report results | 17% | 17% | 17% | 17% |
| | | % Basic Peaks: | % Basic peaks: | % Basic peaks: | % Basic peaks: | % Basic peaks: |
| | | Report results | 25% | 26% | 26% | 25% |
| Safety | Sterility/ | Sterility: Sterile | Sterility: Sterile | Not tested | Not tested | Not tested |
| | Container | CCI: Pass⁶ | | | | |
| | Closure | | | | | |
| | Integrity (CCI) | | | | | |

¹Unless specified, the Time 0 (T0) refers to the batch release data
²Size Exclusion High-Performance Liquid Chromatography
³Capillary Electrophoresis-Sodium Dodecyl Sulfate
⁴Light Chain + Heavy Chain
⁵Cation Exchange High-Pressure Liquid Chromatography
⁶Pass indicates that the sample showed no dye ingress

HMBD-002 Drug Product at 5° C., inverted orientation:

| | | | Timepoints | | | |
|---|---|---|---|---|---|---|
| Attribute | Assay | Acceptance Criteria | T0¹ | 14 D | 1 M | 3 M |
| Appearance | Visual method | Liquid, practically | Liquid, practically | Liquid, practically | Liquid, practically | Liquid, practically |
| | | free from particles | free from particles | free from particles | free from particles | free from particles |
| Color | Visual method | ≤Y3 | <Y6 | <Y6 | <Y6 | <Y6 |
| Clarity | Turbidimetry | ≤Ref. IV | <Ref. III | <Ref. III | <Ref. III | <Ref. III |
| | method | | | | | |
| General | pH | 5.0 to 6.0 | 5.6 | 5.7 | 5.6 | 5.8 |
| | Liquid | ≥10 μm: ≤6000/ | ≥10 μm: | ≥10 μm: | ≥10 μm: | ≥10 μm: |
| | particulate | container | 25 per container | 8 per container | 18 per container | 10 per container |
| | matter | ≥25 μm: ≤600/ | ≥25 μm: | ≥25 μm: | ≥25 μm: | ≥25 μm: |
| | | container | 1 per container | 0 per container | 0 per container | 0 per container |
| Content | Protein content | 42.5 to 57.5 mg/mL | 54.5 mg/mL | 54.2 mg/mL | 52.4 mg/mL | 53.4 mg/mL |
| Potency | Binding assay | 60% to 140% | 84% relative | 105% relative | 87% relative | 84% relative |
| | | relative potency | potency | potency | potency | potency |
| Purity | SE-HPLC² | % Main peak: ≥92% | % Main peak: | % Main peak: | % Main peak: | % Main peak: |
| | | | 95% | 95% | 95% | 95% |
| | CE-SDS³ | % LC + HC⁴ ≥95% | % LC + HC⁴: 98% | % LC + HC⁴: 98% | % LC + HC⁴: 98% | % LC + HC⁴: 98% |
| | (Reduced) | | | | | |
| | CE-SDS³ (Non- | % Main peak: ≥90% | % Main peak: | % Main peak: | % Main peak: | % Main peak: |
| | Reduced) | | 95% | 95% | 95% | 96% |
| | CEX-HPLC⁵ | % Main peak: | % Main peak: | % Main peak: | % Main peak: | % Main peak: |
| | | Report results | 58% | 57% | 58% | 57% |
| | | % Acidic Peaks: | % Acidic peaks: | % Acidic peaks: | % Acidic peaks: | % Acidic peaks: |
| | | Report results | 17% | 17% | 17% | 18% |
| | | % Basic Peaks: | % Basic peaks: | % Basic peaks: | % Basic peaks: | % Basic peaks: |
| | | Report results | 25% | 26% | 25% | 25% |
| Safety | Sterility/ | Sterility: Sterile | Sterile | Not tested | Not tested | Not tested |
| | Container | CCI: Pass⁶ | | | | |
| | Closure | | | | | |
| | Integrity (CCI) | | | | | |

¹Unless specified, the Time 0 (T0) refers to the batch release data
²Size Exclusion High-Performance Liquid Chromatography
³Capillary Electrophoresis-Sodium Dodecyl Sulfate
⁴Light Chain + Heavy Chain
⁵Cation Exchange High-Pressure Liquid Chromatography
⁶Pass indicates that the sample showed no dye ingress

HMBD-002 Drug Product at 25° C., 60% relative humidity:

| | | | Timepoints | | | | |
|---|---|---|---|---|---|---|---|
| Attribute | Assay | Acceptance Criteria | T0¹ | 1 D | 3 D | 1 M | 3 M |
| Appearance | Visual method | Liquid, | Liquid, | Liquid, | Liquid, | Liquid, | Liquid, |
| | | practically free | practically free | practically free | practically free | practically free | practically free |
| | | from particles | from particles | from particles | from particles | from particles | from particles |
| Color | Visual method | ≤Y3 | <Y6 | <Y6 | <Y6 | <Y6 | <Y6 |
| Clarity | Turbidimetry | ≤Ref. IV | <Ref. III | <Ref. III | <Ref. III | <Ref. III | <Ref. III |
| | method | | | | | | |
| General | pH | 5.0 to 6.0 | 5.6 | 5.7 | 5.6 | 5.6 | 5.8 |
| | Ligund | ≥10 μm: ≤6000/ | ≥10 μm: | ≥10 μm: | ≥10 μm: | ≥10 μm: | ≥10 μm: |
| | particulate | container | 25 per container | 13 per container | 15 per container | 45 per container | 42 per container |
| | matter | ≥25 μm: ≤600/ | ≥25 μm: | ≥25 μm: | ≥25 μm: | ≥25 μm: | ≥25 μm: |
| | | container | 1 per container | 0 per container | 0 per container | 1 per container | 0 per container |
| Content | Protein content | 42.5 to 57.5 mg/mL | 54.5 mg/mL | 53.9 mg/mL | 54.3 mg/mL | 52.2 mg/mL | 52.3 mg/mL |
| Potency | Binding assay | 60% to 140% | 84% relative | 100% relative | 96% relative | 95% relative | 91% relative |
| | | relative potency | potency | potency | potency | potency | potency |
| Purity | SE-HPLC² | % Main peak: ≥92% | % Main peak: | % Main peak: | % Main peak: | % Main peak: | % Main peak: |
| | | | 95% | 94% | 94% | 94% | 94% |
| | CE-SDS³ | % LC + HC⁴ ≥95% | % LC + HC⁴: | % LC + HC⁴: | % LC + HC⁴: | % LC + HC⁴: | % LC + HC⁴: |
| | (Reduced) | | 98% | 98% | 98% | 98% | 98% |
| | CE-SDS³ | % Main peak: ≥90% | % Main peak: | % Main peak: | % Main peak: | % Main peak: | % Main peak: |
| | (Non- | | 95% | 95% | 95% | 95% | 95% |
| | Reduced) | | | | | | |
| | CEX-HPLC⁵ | % Main peak: | % Main peak: | % Main peak: | % Main peak: | % Main peak: | % Main peak: |
| | | Report results | 58% | 58% | 58% | 58% | 56% |
| | | % Acidic Peaks: | % Acidic peaks: | % Acidic peaks: | % Acidic peaks: | % Acidic peaks: | % Acidic peaks: |
| | | Report results | 17% | 17% | 17% | 16% | 19% |
| | | % Basic Peaks: | % Basic peaks: | % Basic peaks: | % Basic peaks: | % Basic peaks: | % Basic peaks: |
| | | Report results | 25% | 25% | 25% | 26% | 25% |
| Safety | Sterility | Sterile | Sterile | Not tested | Not tested | Not tested | Not tested |

¹Unless specified, the Time 0 (T0) refers to the batch release data
²Size Exclusion High-Performance Liquid Chromatography
³Capillary Electrophoresis-Sodium Dodecyl Sulfate
⁴Light Chain + Heavy Chain
⁵Cation Exchange High-Pressure Liquid Chromatograph

HMBD-002 Drug Product at 40° C., 75% relative humidity:

| | | | Timepoints | | | |
|---|---|---|---|---|---|---|
| Attribute | Assay | Acceptance Criteria | T0¹ | 7 D | 14 D | 1 M |
| Appearance | Visual method | Liquid, | Liquid, | Liquid, | Liquid, | Liquid, |
| | | practically free | practically free | practically free | practically free | practically free |
| | | from particles | from particles | from particles | from particles | from particles |
| Color | Visual method | ≤Y3 | <Y6 | <Y6 | <Y6 | <Y6 |
| Clarity | Turbidimetry | ≤Ref. IV | <Ref. III | <Ref. III | <Ref. III | <Ref. III |
| | method | | | | | |
| General | pH | 5.0 to 6.0 | 5.6 | 5.7 | 5.7 | 5.7 |
| | Liquid particulate | ≥10 μm: ≤6000/ | ≥10 μm: | ≥10 μm: | ≥10 μm: | ≥10 μm: |
| | matter | container | 25 per container | 15 per container | 49 per container | 62 per container |
| | | ≥25 μm: ≤600/ | ≥25 μm: | ≥25 μm: | ≥25 μm: | ≥25 μm: |
| | | container | 1 per container | 0 per container | 0 per container | 1 per container |
| Content | Protein content | 42.5 to 57.5 mg/mL | 54.5 mg/mL | 53.2 mg/mL | 54.0 mg/mL | 51.9 mg/mL |
| Potency | Binding assay | 60% to 140% | 84% relative | 99% relative | 98% relative | 87% relative |
| | | relative potency | potency | potency | potency | potency |
| Purity | SE-HPLC² | % Main peak: ≥92% | % Main peak: | % Main peak: | % Main peak: | % Main peak: |
| | | | 95% | 93% | 93% | 93% |
| | CE-SDS³ | % LC + HC⁴ ≥95% | % LC + HC⁴: 98% | % LC + HC⁴: 98% | % LC + HC⁴: 97% | % LC + HC⁴: 97% |
| | (Reduced) | | | | | |
| | CE-SDS³ (Non- | % Main peak: ≥90% | % Main peak: | % Main peak: | % Main peak: | % Main peak: |
| | Reduced) | | 95% | 95% | 94% | 93% |
| | CEX-HPLC⁵ | % Main peak: | % Main peak: | % Main peak: | % Main peak: | % Main peak: |
| | | Report results | 58% | 57% | 56% | 45% |
| | | % Acidic Peaks: | % Acidic peaks: | % Acidic peaks: | % Acidic peaks: | % Acidic peaks: |
| | | Report results | 17% | 15% | 16% | 27% |
| | | % Basic Peaks: | % Basic peaks: | % Basic peaks: | % Basic peaks: | % Basic peaks: |
| | | Report results | 25% | 28% | 28% | 28% |
| Safety | Sterility | Sterile | Sterile | Not tested | Not tested | Sterile |

¹Unless specified, the Time 0 (T0) refers to the batch release data
²Size Exclusion High-Performance Liquid Chromatography
³Capillary Electrophoresis-Sodium Dodecyl Sulfate
⁴Light Chain + Heavy Chain
⁵Cation Exchange High-Pressure Liquid Chromatography

HMBD-002 Drug Product subjected to freeze/thaw conditions:

| | | | | Freeze and Thaw |
|---|---|---|---|---|
| Attribute | Assay | Acceptance Criteria | T0¹ | 6 cycles, −20° C. to 25° C. |
| Appearance | Visual method | Liquid, practically | Liquid, practically | Liquid, practically |
| | | free from particles | free from particles | free from particles |
| Color | Visual method | ≤Y3 | <Y6 | <Y6 |
| Clarity | Turbidimetry method | ≤Ref. IV | <Ref. III | <Ref. III |
| General | pH | 5.0 to 6.0 | 5.6 | 5.7 |
| | Liquid particulate | ≥10 μm: ≤6000/container | ≥10 μm: | ≥10 μm: |
| | matter | | 25 per container | 12 per container |
| | | ≥25 μm: ≤600/container | ≥25 μm: | ≥25 μm: |
| | | | 1 per container | 0 per container |
| Content | Protein content | 42.5 to 57.5 mg/mL | 54.5 mg/mL | 53.5 mg/mL |
| Potency | Binding assay | 60% to 140% relative | 84% relative | 86% relative |
| | | potency | potency | potency |
| Purity | SE-HPLC² | % Main peak: ≥92% | % Main peak: 95% | % Main peak: 95% |
| | CE-SDS³ (Reduced) | % LC + HC⁴ ≥ 95% | % LC + HC⁴: 98% | % LC + HC⁴: 98% |
| | CE-SDS³ (Non- | % Main peak: ≥90% | % Main peak: 95% | % Main peak: 95% |
| | Reduced) | | | |
| | CEX-HPLC⁵ | % Main peak: | % Main peak: 58% | % Main peak: 57% |
| | | Report results | | |
| | | % Acidic Peaks: | % Acidic peaks: 17% | % Acidic peaks: 17% |
| | | Report results | | |
| | | % Basic Peaks: | % Basic peaks: 25% | % Basic peaks: 26% |
| | | Report results | | |
| Safety | Sterility | Sterile | Sterile | Sterile |

¹Unless specified, the Time 0 (T0) refers to the batch release data
²Size Exclusion High-Pressure Liquid Chromatography
³Capillary Electrophoresis-Sodium Dodecyl Sulfate
⁴Light chain + Heavy chain
⁵Cation Exchange High-Pressure Liquid Chromatography

The results generated demonstrate that the drug product remains stable at the recommended storage conditions up to 6 months. Similar results to those above were also seen at the 6-month and 9-month time points for each agent/condition. All stability data generated to the 9-month time point remained within the established specification acceptance criteria. Based on the stability results the assigned expiry period will be 12 months.

### Example 19: A Phase 1 Study of HMBD-002-V4C26, a Monoclonal Antibody Targeting VISTA, in Patients with Advanced Solid Malignancies

As used hereinbelow, HMDB-002 or HMBD-002-V4C26 refers to an IgG4 humanised monoclonal antigen-binding molecule comprising V4-C26 variable regions in human IgG4/Vκ format, and which is comprised of the heavy chain of SEQ ID NO:331 and the light chain of SEQ ID NO:317.

### 19.1 INTRODUCTION

VISTA is a transmembrane immunomodulatory glycoprotein of the B7 protein family that functions as a negative immune checkpoint regulator. Its closest homolog is PD-L1, with which it shares 24% sequence homology.¹ VISTA is only distantly related to other members of the Ig superfamily.^{1,2}

The recently identified crystal structure of VISTA has highlighted features that make the VISTA IgV-like fold unique among B7 family members. These include the presence of 10 beta strands (instead of the canonical 9), 3 alpha helices arranged in a beta-sandwich formation, and a unique 21-residue turn between strands C and C′ that forms an extended loop. The crystal structure also shows that VISTA lacks the IgC domain, which is present in all the other B7 family proteins that have been crystallized. Lastly, VISTA contains 2 non-canonical and conserved C51/C113 disulfides, one of which likely holds the extended C-C′ loop in a unique, protruding position that may play a role in clinically relevant protein-protein interactions.³

Early pre-clinical studies showed that recombinant VISTA treatment or VISTA expression on antigen-presenting cells (APCs) was sufficient to repress T cell proliferation, which was independent of the PD-1 pathway.¹ Additionally, multiple studies have shown that VISTA constrains autoimmunity in numerous pre-clinical models including psoriasis, auto-immune encephalitis, lupus, and graft-versus-host disease.^{1,2,5,6,7,8} Mice lacking the VISTA gene develop organ and skin autoimmunity, phenotypes similar to PD-1 pathway de-regulation.^{5,9,10,11} These data indicate that VISTA functions to suppress immune responses, likely through actions on lymphocyte and myeloid cells, especially naïve T cells.^{12,13} Thus, VISTA is a transmembrane glycoprotein that functions as a negative immune checkpoint regulator.

VISTA is predominantly expressed on myeloid and granulocytic cells, especially MDSC and tumor associated macrophages (TAM). VISTA has also been reported to be expressed on CD4+ T regulatory cells, naïve T cells, as well as some tumor cells.^{14,15,16} Immunosuppression by MDSC and upregulation of VISTA has been linked to acquired resistance to anti-cytotoxic T-lymphocyte-associated protein 4 (CTLA4) and anti-PD-1/PD-L1 therapies in solid cancers and in acute myeloid leukemia.^{17,18,19} VISTA knockdown has been reported to potently reduce MDSC-mediated CD8 T cell activity.¹⁹ Additionally, VISTA has been reported to play a key role in maintaining naïve T cell quiescence and peripheral immune tolerance¹⁵. Loss of VISTA on these cells reduced the quiescence phenotype of the naïve T cells at the transcriptional and epigenetic levels, enhancing both T cell receptor (TCR) and cytokine pathway activities.

The expression of VISTA on naïve T cells precedes the expression of other well established negative checkpoint regulators of T cell activation, such as CTLA4 and PD-1.¹³ CTLA4 is expressed on the cell surface after T cell activation and inhibits T cell activation at the priming stage by limiting co-stimulation. PD-1 is expressed later during priming and inhibits T cells at the effector stage, and VISTA functions as the earliest checkpoint regulator of peripheral T cell tolerance.¹³

Based on the similarities between VISTA gene function and the PD-L1 pathway, the role of VISTA in tumor immunity is becoming better understood. Initial studies showed that VISTA over-expression on tumor cells was sufficient to protect against anti-tumor immunity in mice.¹ Blocking antibodies to murine VISTA were then developed and utilized to demonstrate that VISTA blockade could spur anti-tumor immunity through actions on the tumor microenvironment (TME). 18 Antibodies that block VISTA function have shown synergy with PD-1 blockade, again implying that VISTA and PD-1 function independently¹⁰. Anti-tumor responses to VISTA blockade have been demonstrated in pre-clinical murine models of lymphoma, bladder cancer, melanoma, colorectal cancer, ovarian cancer, head and neck squamous cell carcinoma, and other malignancies, indicating that this approach may have broad utility. Also, human tumors including melanoma, pancreatic cancer, mesothelioma, NSCLC, uterine, breast, and prostate express VISTA either on tumor cells themselves or immune infiltrating cells.^{1,12,14,15,18,19,21}-34 Based on this set of information, it serves to reason that VISTA may be an actionable target across multiple human malignancies.

The physiologically relevant binding partners of VISTA are yet to be conclusively determined, but two independent in vitro studies have demonstrated V-Set and Immunoglobulin domain containing 3 (VSIG3, or IGSF11) as a partner.^{3,20} VSIG3 is expressed on cancer cells with elevated expression reported in multiple malignances including colorectal, hepatocellular, and intestinal-type gastric cancers²⁰.

Additionally, the VISTA-VSIG3 interaction has been shown to inhibit T cell proliferation, as well as pro-inflammatory cytokine and chemokine release.²⁰ VISTA has also been reported to bind to itself⁹, leucine-rich repeats and immunoglobulin-like domains 1 (LRIG1)³⁵, and P-selectin glycoprotein-1 (PSGL-1) at an acidic pH.^{4,37}

Based on the concept of immune checkpoint inhibitors as broadly active agents against multiple solid tumors, efforts have recently focused on translating VISTA-targeting therapies to the clinical realm. There are 3 clinical-stage investigational therapeutics that target VISTA but employ different drug mechanisms than HMBD-002. Onvatilimab (CI-8993), a Phase 1 clinical-stage anti-VISTA antibody with an IgG1 isotype, is expected to cause depletion of VISTA+ cells, including normal hematopoietic cells. This compound is currently being developed by Curis, Inc. in partnership with Immunext. In 2016, Janssen Research & Development, in partnership with Immunext, initiated a non-randomized, open-label Phase 1 study to evaluate the safety of onvatilimab.³⁹ This study enrolled 12 patients and then the program was suspended until June 2020, when Curis, Inc. reinitiated a Phase 1 study to evaluate the safety and tolerability of onvatilimab.⁴⁰ Publicly available information suggests that patient recruitment is on-going.

In November 2020, Pierre Fabre initiated a Phase 1 clinical study⁴¹ of the anti-VISTA mAb, W0180, also an IgG1 isotype, as a monotherapy and combined with pembrolizumab in patients with locally advanced or metastatic solid tumors, refractory to standard therapies. Recruitment in this study is ongoing.

Another clinical-stage therapeutic targeting VISTA, CA-170, is a small molecule that was reported to bind PD-L1 and VISTA.³⁴ As an oral small molecule with multi-target antagonism, this compound has a different mechanism of action compared to HMBD-002, which has high specificity to only the VISTA protein. CA-170 was initially discovered by Aurigene, and developed by Curis, Inc, until development was discontinued. A third-party study reported in ACS Medicinal Chemistry Letters in 2019 was unable to reproduce binding of the compound to VISTA, suggesting an alternate mechanism of action for the compound.³⁵ In a Phase 1 dose-escalation study conducted by Curis, Inc.⁴², the drug was well-tolerated with mostly immune-related adverse events. 36

Taken together, there is limited human experience in targeting VISTA, and current approaches represent substantially different mechanisms of action compared to HMBD-002.

### 19.2 STUDY RATIONALE

### 19.2.1 Rationale for Study Design

There is a clinical unmet need for new therapies and VISTA serves as an attractive immunotherapy target due to its robust inhibitory activities. The rationale for targeting VISTA is further strengthened by the results of preclinical studies, indicating that prior treatment with anti-PD-1 mAbs potentially render VISTA a more important target. In contrast to other anti-VISTA mAbs, HMBD-002 is an IgG4 construct and therefore would not be expected to induce ADCC after binding to T cells, which is the putative reason for CRS and/or related neurotoxicity observed with other IgG1 antibodies. In syngeneic VISTA-expressing tumour models, HMBD-002 treatment profoundly inhibited tumour growth and/or resulted in tumour regression.

The first part (Part 1; Dose Escalation) of this FIH study of HMBD-002 is designed to assess the safety and tolerability of HMBD-002 and to recommend a dose for Part 2 (Dose Expansion) and subsequent disease-directed Phase 2 studies. The study also seeks to characterize the PK behaviour of HMBD-002 and detect HMBD-002 ADAs as well as to seek preliminary evidence of anti-cancer activity in patients with solid malignancies with an expansion in select malignancies with a high incidence of VISTA expression, including advanced triple negative breast cancer (TNBC) and non-small cell lung cancer (NSCLC), and various other cancers. This study will also explore the effects of HMBD-002 on immune effector cells in tumours and blood sampled before and after treatment.

An integrated, data driven approach that includes the available in vitro, pharmacologically active dose (PAD), modelled and expected receptor occupancy (RO) and toxicology data has been used to identify and support an appropriate FIH starting dose. The FIH starting dose of approximately 20 mg (˜0.3 mg/kg) with an intended ‘test’ dose of 33% or 7 mg (0.1 mg/kg) is proposed. The test dose is expected to attain maximal concentration (Cₘₐₓ) of 2.2 μg/mL and between 16.4 to 18.3% receptor occupancy in female and male humans, respectively.

The starting dose is based on the PAD, which is determined using the average serum concentration of 27.2 μg/mL (attained from the PK data of syngeneic tumour bearing mice treated with the lowest minimally efficacious dose of 5 mg/kg administered twice weekly, see Example 19.2.4), a predicted total human clearance (CL) of 53 mL/day (calculated using allometric scaling of cynomolgus monkey parameters from GLP toxicology study) and an assumed bioavailability of 1, as the drug will be delivered intravenously. The PAD approach supports a starting dose of approximately 10 mg (0.17 mg/kg).

The starting dose was also based on the lowest concentration of 1 μg/mL tested in the functional allogenic mixed lymphocyte reaction (MLR) mixed assay where minimal to no changes in cytokines were observed compared to the isotype control. The allogenic MLR assay used 10 pairs of single donor sourced peripheral blood mononuclear cells (PBMCs) which we were mixed, then treated with antibody HMBD-002 or hIgG4 isotype control. HMBD-002 induced a significant dose dependent increase in the levels of IFN-γ, TNF-α and IL-17A while there were no significant changes compared to isotype control in the levels of IL-4, IL-10 and IL-13 (Th2 cytokines) or IL-6 at 96 hrs. The expected (allometrically scaled) human central volume of distribution of 3.52 L, and using the 1 μg/mL as expected Cₘₐₓ, suggests a starting dose of 3.5 mg (˜0.05 mg/kg).

The proposed dose is also supported by 1-month IV rat and monkey repeat dose toxicity studies. Compared to the projected human equivalent dose (HED) of approximately 16.7 mg/kg, the proposed FIH starting dose is anticipated to give a significant (167-fold) safety margin relative to the NOAEL/HNSTD and the NOAEL/STD10 for monkeys and rat, respectively at 100 mg/kg.

The proposed FIH starting dose of 20 mg along with the 7 mg (test) dose will ensure adequate assessment of PK, PD, safety and efficacy of HMBD-002 in humans. All patients in Cohort 1 of the dose escalation stage will receive 33% of the intended dose level as a “test” dose on Day 1 (e.g., 7 mg on Day 1 of Cycle 1 of Cohort 1) with all subsequent doses administered as the intended cohort dose (e.g., 20 mg on Days 8 and 15 [cycle 1], as well as on Days 1, 8, and 15 [cycles 2+]).

### 19.2.2 Rationale for Clinical Indications

This is a Phase 1, first-in-human (FIH), two-stage (Part 1: dose escalation and Part 2: dose expansion) study evaluating multiple doses and schedules of intravenously (IV) administered HMBD-002 in patients with advanced solid tumours (i.e., locally advanced and unresectable, or metastatic).

Based on the parallels between VISTA expression in human cancer and its role in cancer immunity, e.g. as an immune-suppressive factor within the tumour microenvironment, two priority indications have been chosen for early clinical stage trials: triple negative breast cancer (TNBC) and non-small cell lung cancer (NSCLC).

TNBC has been identified as a key cancer type for clinical investigation of HMBD-002 based on the unmet need and widespread expression of VISTA in cancer of this type.^{35,39} The state-of the art TNBC treatment is the approved combination of atezolizumab and chemotherapy, which is indicated for patients with local or metastatic cancer with tumors that express PD-L1. However, only 40% of patients with TNBC have malignancies that express PD-L1, leaving the majority of advanced TNBC patients unable to pursue this option.⁴⁰

An IHC study of over 900 patients with invasive ductal carcinoma revealed a high level of expression of VISTA on immune cells in ˜30% of the cases.³⁹ In 8% of the cases, VISTA was expressed on the cancer cells themselves. Further, the expression of VISTA is enriched in some subsets of breast cancer. ER−, PR−, and HER2+ breast cancers were found to more frequently express VISTA than ER+ and/or PR+ expressing breast cancer. The subset with the greatest enrichment for VISTA expression was TNBC, with greater than 45% of patients scored with VISTA-positive cancer.³⁹ In a separate study, 20% of TNBC cases showed moderate-to-high intensity of staining for VISTA protein on cancer cells and infiltrating immune cells.³⁵ Notably, all TNBC sampled from metastases expressed uniform high levels of VISTA.³⁵ Hummingbird's internal analysis of TNBC tissue microarrays have also shown that of the 119 cores analyzed, 89% expressed VISTA, and of these, 50% showed moderate-to-high staining intensity. Given the evidence for the immune modulatory role of VISTA and evidence of elevated VISTA levels in TNBC patient samples, especially metastatic TNBC, it is a rational priority indication.

Similarly, in NSCLC, immune checkpoint inhibitors such as mAbs to CTLA-4 and PD-1/PD-L1, have demonstrated robust anti-tumor activity and clinical benefit; however, there is still room for significant improvement which might require overcoming alternative immune checkpoints⁴¹. High levels of VISTA expression in NSCLC have been well documented. In a study of 636 resected NSCLC tumor samples, VISTA protein was detected (predominantly in stromal cells) in 99% of cases.²⁷ Examining the spatial patterns in the tumor immune infiltrate, VISTA was positively associated with PD-L1, PD-1, CD8+ T cells and CD68+ macrophages.²⁷ In support of this, analysis of NSCLC tissue microarrays has also shown that of the 69 cores analyzed, 85% expressed VISTA. It has been hypothesized that VISTA may contribute to NSCLC treatment resistance via its expression on MDSCs, the levels of which were elevated in tumor and peripheral blood in patients with NSCLC, adding further support for the potential benefit of targeting VISTA.

In the current first-in-human study, targeting VISTA with HMBD-002 may induce broad and robust therapeutic activity. This study will first focus on solid tumors and target patients with advanced TNBC, NSCLC, and various advanced cancers known to express VISTA. Potential patients eligible for enrollment in this FIH study must have evidence of a malignant solid tumor and must have advanced disease, defined as cancer that is either metastatic or locally advanced and unresectable. Patients must also have disease in which available therapies are unlikely to impact favorably and confer clinical benefit.

### 19.2.3 Nonclinical Studies of HMBD-002

In vitro and in vivo pharmacology and preliminary tolerability studies have been conducted to investigate the potential therapeutic benefits and toxicology risk profile for HMBD-002 for the treatment of patients with VISTA-expressing malignancies. Studies have been completed to demonstrate the affinity and specificity of HMBD-002 for VISTA and to establish the mechanism of action through ligand inhibition and cytokine induction. These studies have shown that HMBD-002 does not bind to FcγRIII and C1q, and demonstrated that HMBD-002 has notable antitumor activity as either monotherapy or combined with various checkpoint inhibitors in several murine syngeneic tumor (CDX) models.

Enabled by the cross-species binding of HMBD-002, with comparable affinity to VISTA orthologs in pre-clinical toxicology species, single dose PK studies in mice along with PK and tolerability studies in rats and non-human primates (NHP) have been completed. The data from these studies have been used to preliminarily indicate safety and to calculate potential frequency and dose for this FIH study.

### 19.2.4 Preclinical Efficacy

Efficacy in an in vitro model of human immune activation was evaluated using healthy donor human peripheral blood mononuclear cells (PBMCs). An allogeneic Mixed Lymphocyte Reaction (MLR) assay was used where PBMCs from separate donors were mixed in pairs to model a self/anti-self-immune response. HMBD-002 was demonstrated to enhance the activation of pro-inflammatory signals, as measured by cytokine release, including gamma interferon (IFNγ) and tumor necrosis factor-α (TNFα).

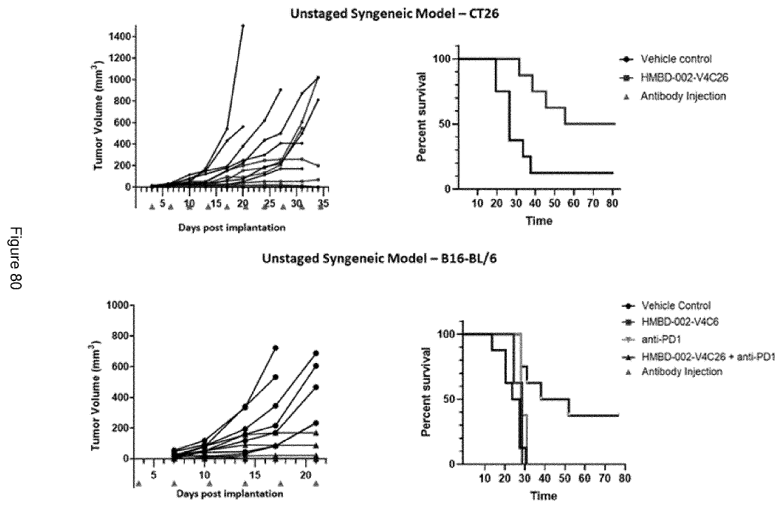

To assess the in vivo efficacy of HMBD-002, murine syngeneic tumor models CT26 (colon carcinoma) and B16/BL6 (melanoma) were treated with HMBD-002 as a monotherapy and in combination with a functional anti-murine PD-1 mAb. Mice were treated with either 500 μg (˜25 mg/kg) of HMBD-002 alone or in combination with 200 μg (˜10 mg/kg) of anti-mPD-1 (RMP1-14). As seen in FIG. 80, in a CT26 monotherapy study, HMBD-002 significantly inhibited tumor growth and improved survival (6 of 8 mice with tumor volume below 200 mm³ for the HMBD-002 treated arm at study termination, compared to the vehicle arm with only 1 of 8 mice with tumor volume below 200 mm³).

In the B16/BL6 model, HMBD-002 in combination with anti-PD-1 treatment significantly inhibited tumor growth (7 of 8 mice with tumor volume below 200 mm³ for the combination treated arm at study termination, compared to the vehicle arm with only 1 of 8 mice with tumor volume below 200 mm³).

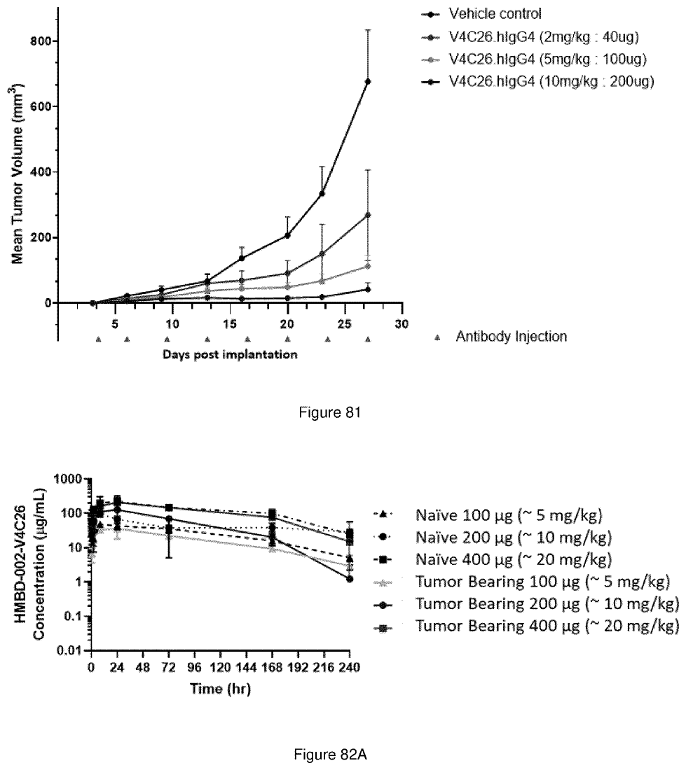

To elucidate the minimum effective dose of HMBD-002, the CT26 tumor model was selected and mice were treated with different doses of HMBD-002. As seen in FIG. 81, HMBD-002 shows efficacy at doses of 100 μg (˜5 mg/kg), as well as higher doses. These results can be used to support calculations of a pharmacologically active starting dose.

### 19.2.5 Toxicology and Pharmacology

To support the clinical evaluation of HMBD-002, the toxicity of HMBD-002 was assessed in a series of non-clinical in vitro assays, and in vivo studies in the Sprague-Dawley rat and cynomolgus monkeys. The rat and monkey were selected as the pharmacologically relevant species because of homology to the human VISTA protein sequence and similar binding affinity of HMBD-002 to rat and monkey VISTA, as compared to human.

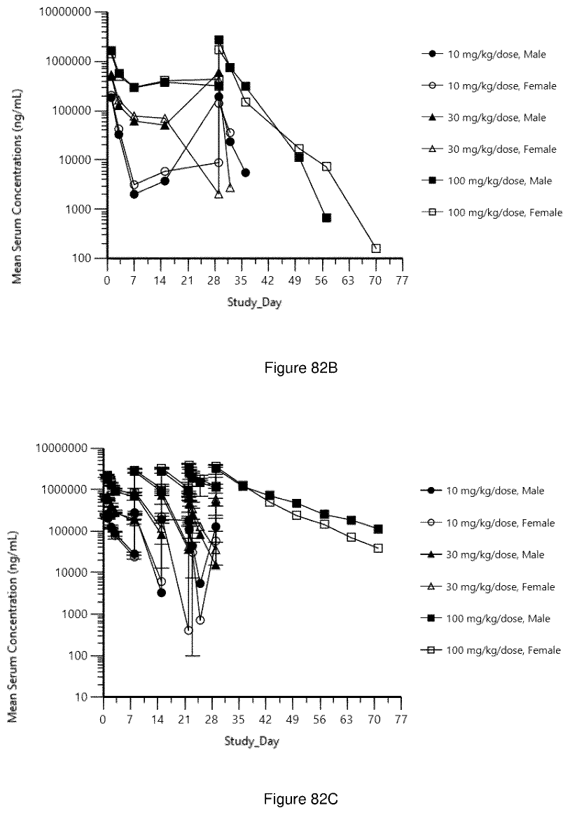

In mice, the PK profile was determined from measurements in tumour-bearing and non-tumour bearing mice with different doses of HMBD-002. FIG. 82A shows that the PK profiles of HMBD-002 in the tumour bearing mice were non-linear, while non-tumour bearing mice demonstrated linear PK.

Near dose-proportional exposure was observed in the non-tumour bearing mice, as measured by Cmax and AUCinf. This was also observed with Cmax in the tumour-bearing mice, however the AUCinf in tumour-bearing mice demonstrated a slightly more than dose proportional increase in exposure. HMBD-002 serum half-life in the tumour-bearing mice was calculated as 29.6 to 58.6 hours across doses, while in the non-tumour bearing mice, serum half-life was 60.9 and 178 hours.

In rats, the PK profile was determined from measurements in male and female Sprague-Dawley rats in a study with HMBD-002 at weekly doses of 10 mg/kg, 30 mg/kg, and 100 mg/kg for 5 doses. The PK profiles of HMBD-002 did not differ between genders. FIG. 82B shows that, as the dosage increased from 10 to 100 mg/kg/dose, the systemic exposure increased dose proportionally in both genders on Day 1 and in females on Day 29 while it increased more than dose proportionally in males on Day 29. No marked drug accumulation for HMBD-002 was observed at any dose level.

In cynomolgus monkeys, the PK profile was determined from measurements in male and female monkeys in a study with HMBD-002 at weekly doses of 10 mg/kg, 30 mg/kg, and 100 mg/kg for a total of 5 doses. FIG. 82C shows that the PK profile of HMBD-002 did not differ between the genders.

Due to the detected anti-drug antibody (ADA) titers on Day 22, the results on Day 22 should be interpreted with caution. The mean±SD for all PK parameters of HMBD-002-V4C26 following once weekly IV injection of HMBD-002-V4C26 at 10, 30, or 100 mg/kg/dose to male and female monkeys for a total of 5 doses are presented in Table 5.

Due to ADA presence and its influence on exposure at the lower doses, AUC_{0-168 h} increased more than dose proportionally in both genders on Day 22. After single (Day 1) or repeat (Day 22) IV injection of HMBD-002-V4C26 to male and female monkeys, median T_{1/2} values for HMBD-002-V4C26 were estimated between 59.6 and 177.3 hours on Day 1 and between 7.1 and 138.0 hours on Day 22 which might result from the influence of ADA (Table 1). T_{1/2} values were estimated at 35.3 and 70.6 hours for ADA positive males and females, respectively on Day 29 (Table 2) and 285.1 and 186.1 hours for ADA negative males and females, respectively on Day 29 (Table 3).

Median half-life values were estimated between 59.6 and 177.3 hours on Day 1 and between 7.1 and 250 hours on Day 22 which might result from the influence of ADA.

TABLE 1
Summary of TK Parameters of HMBD-002-V4C26 Following Multiple Doses in All Study Animals

| | | | | | | DNAUCₐₗₗ | | | | | | | |
|---|---|---|---|---|---|---|---|---|---|---|---|---|---|
| Dose | | | | | | ((h*ug/mL)/ | | | CL | | | | |
| (mg/kg/ | Study | | Cₘₐₓ | T_{1/2} | AUCₐₗₗ | (mg/kg/ | AUC₀₋ₗₐₛₜ | AUC_{0-inf} | (mL/ | Vss | Tₘₐₓ | Tₗₐₛₜ | Cₗₐₛₜ |
| dose) | Day | Sex | (ug/mL) | (h) | (h*ug/mL) | dose)) | (h*ug/mL) | (h*ug/mL) | h/kg) | (mL/kg) | (h) | (h) | (ug/mL) |
| 10 | 1 | Male | 243 ± | 65.7 | 14200 ± | 1420 ± | 14200 ± | 16900 ± | 0.593 ± | 54.8 ± | 1.0 | 168.0 | 28.4 ± |
| | | | 11.3 | (58.4- | 978 | 97.8 | 978 | 764 | 0.0270 | 6.21 | (1.0- | (168.0- | 26.1 |
| | | | | 73.5) | | | | | | | 1.0) | 168.0) | |
| | | Female | 258 ± | 59.6 | 13100 ± | 1310 ± | 13100 ± | 15100 ± | 0.666 ± | 55.0 ± | 1.0 | 168.0 | 23.9 ± |
| | | | 19.4 | (55.2- | 1210 | 121 | 1210 | 1470 | 0.0621 | 3.44 | (1.0- | (168.0- | 2.95 |
| | | | | 59.7) | | | | | | | 1.0) | 168.0) | |
| | 22 | Male | 181 ± | 7.1 | 3040 ± | 304 ± | 2930 ± | 2990 ± | 5.16 ± | 68.6 ± | 1.0 | 72.0 | 4.77 ± |
| | | | 49.2 | (6.4- | 2540 | 254 | 2640 | 2620 | 3.20 | 17.3 | (1.0- | (24.0- | 7.23 |
| | | | | 22.6) | | | | | | | 1.0) | 168.0) | |
| | | Female | 168 ± | 9.3 | 2130 ± | 213 ± | 2060 ± | 2080 ± | 5.97 ± | 69.5 ± | 1.0 | 72.0 | 2.64 ± |
| | | | 37.6 | (5.5- | 1060 | 106 | 1140 | 1110 | 3.42 | 16.1 | (1.0- | (24.0- | 3.96 |
| | | | | 14.0) | | | | | | | 1.0) | 168.0) | |
| 30 | 1 | Male | 667 ± | 150.1 | 49700 ± | 1660 ± | 49700 ± | 91900 ± | 0.327 ± | 70.3 ± | 1.0 | 168.0 | 193 ± |
| | | | 67.9 | (147.9- | 1050 | 35.0 | 1050 | 2660 | 0.00946 | 1.63 | (1.0- | (168.0- | 5.51 |
| | | | | 155.3) | | | | | | | 1.0) | 168.0) | |
| | | Female | 723 ± | 110.7 | 50800 ± | 1690 ± | 50800 ± | 78300 ± | 0.388 ± | 61.2 ± | 1.0 | 168.0 | 169 ± |
| | | | 54.9 | (99.5- | 4350 | 145 | 4350 | 10500 | 0.0565 | 3.70 | (1.0- | (168.0- | 24.3 |
| | | | | 125.4) | | | | | | | 1.0) | 168.0) | |
| | 22 | Male | 627 ± | 20.0 | 19600 ± | 653 ± | 19600 ± | 20600 ± | 1.94 ± | 56.9 ± | 1.0 | 168.0 | 15.2 ± |
| | | | 64.5 | (14.6- | 12200 | 407 | 12200 | 13900 | 1.14 | 2.14 | (1.0- | (168.0- | 24.6 |
| | | | | 46.4) | | | | | | | 1.0) | 168.0) | |
| | | Female | 722 ± | 20.2 | 28600 ± | 953 ± | 28600 ± | 32000 ± | 1.49 ± | 42.5 ± | 1.0 | 168.0 | 37.3 ± |
| | | | 84.3 | (9.5- | 19800 | 660 | 19800 | 25400 | 1.15 | 3.87 | (1.0- | (72.0- | 59.5 |
| | | | | 66.0) | | | | | | | 1.0) | 168.0) | |
| 100 | 1 | Male | 2090 ± | 177.3 | 164000 ± | 1640 ± | 164000 ± | 312000 ± | 0.334 ± | 71.3 ± | 1.0 | 168.0 | 675 ± |
| | | | 285 | (106.7- | 16400 | 164 | 16400 | 77600 | 0.0874 | 10.4 | (1.0- | (168.0- | 120 |
| | | | | 183.7) | | | | | | | 1.0) | 168.0) | |
| | | Female | 2230 ± | 150.1 | 183000 ± | 1830 ± | 183000 ± | 367000 ± | 0.279 ± | 61.3 ± | 1.0 | 168.0 | 799 ± |
| | | | 50.0 | (103.3- | 11300 | 113 | 11300 | 67100 | 0.0554 | 8.58 | (1.0- | (168.0- | 59.3 |
| | | | | 209.4) | | | | | | | 1.0) | 168.0) | |
| | 22 | Male | 3280 ± | 68.2 | 262000 ± | 2620 ± | 262000 ± | 614000 ± | 0.510 ± | 49.7 ± | 1.0 | 168.0 | 1130 ± |
| | | | 810 | (46.0- | 139000 | 1390 | 139000 | 819000 | 0.410 | 12.5 | (1.0- | (168.0- | 884 |
| | | | | 380.7) | | | | | | | 1.0) | 168.0) | |
| | | Female | 3870 ± | 138.0 | 307000 ± | 3070 ± | 307000 ± | 590000 ± | 0.173 ± | 38.7 ± | 1.0 | 168.0 | 1200 ± |
| | | | 354 | (126.3- | 15800 | 158 | 15800 | 90200 | 0.0264 | 4.26 | (1.0- | (168.0- | 131 |
| | | | | 223.1) | | | | | | | 1.0) | 168.0) | |
| | 29 | Male | 3260 | 177.9 | 714000 | 7140 | 714000 | 764000 | 0.199 | 37.1 | 1.0 | 840.0 | 114 |
| | | Female | 3520 | 110.7 | 623000 | 6230 | 623000 | 633000 | 0.161 | 31.3 | 1.0 | 924.0 | 39.0 |

Data is presented as mean or mean ± SD for Cₘₐₓ, Cₗₐₛₜ, DNAUC, Cl, Vss and AUC values, and mean or median (range) for T_{1/2}, Tₘₐₓ and Tₗₐₛₜ.
AUCₐₗₗ: AUC_{0-168 h} for Days 1 and 22 and AUC_{0-1008 h} for Day 29

No marked drug accumulation was observed at 100 mg/kg/dose. However, due to ADA presence and its influence on overall exposure, decreased systemic exposure was observed at 10 and 30 mg/kg/dose generally.

Cₘₐₓ and AUC_{0-24 h} and AUC_{0-72 h} have been summarized in Table 2.

TABLE 2
Summary of TK Parameters of HMBD-002-V4C26 Following Multiple Doses in ADA Positive Study Animals

| Dose | | | | | | DNAUCₐₗₗ | | | | | | | |
|---|---|---|---|---|---|---|---|---|---|---|---|---|---|
| (mg/kg/ | Study | | Cₘₐₓ | T_{1/2} | AUCₐₗₗ | ((h*ug/mL)/ | AUC₀₋ₗₐₛₜ | AUC_{0-inf} | CL | Vss | Tₘₐₓ | Tₗₐₛₜ | Cₗₐₛₜ |
| dose) | Day | Sex | (ug/mL) | (h) | (h*ug/mL) | (mg/kg/dose)) | (h*ug/mL) | (h*ug/mL) | (mL/h/kg) | (mL/kg) | (h) | (h) | (ug/mL) |
| 10 | 1 | Male | 237 | 65.7 | 13600 | 1360 | 13600 | 16100 | 0.621 | 56.6 | 1.0 | 168.0 | 26.5 |
| | 22 | Male | 181 ± | 7.1 | 3040 ± | 304 ± | 2930 ± | 2990 ± | 5.16 ± | 68.6 ± | 1.0 | 72.0 | 4.77 ± |
| | | | 49.2 | (6.4- | 2540 | 254 | 2640 | 2620 | 3.20 | 17.3 | (1.0- | (24.0- | 7.23 |
| | | | | 22.6) | | | | | | | 1.0) | 168.0) | |
| | | Female | 168 ± | 9.3 | 2130 ± | 213 ± | 2060 ± | 2080 ± | 5.97 ± | 69.5 ± | 1.0 | 72.0 | 2.64 ± |
| | | | 37.6 | (5.5- | 1060 | 106 | 1140 | 1110 | 3.42 | 16.1 | (1.0- | (24.0- | 3.96 |
| | | | | 14.0) | | | | | | | 1.0) | 168.0) | |
| 30 | 1 | Female | 691 | 112.45 | 49000 | 1630 | 49000 | 75500 | 0.4035 | 63.3 | 1.0 | 168.0 | 161 |
| | 22 | Male | 627 ± | 20.0 | 19600 ± | 653 ± | 19600 ± | 20600 ± | 1.94 ± | 56.9 ± | 1.0 | 168.0 | 15.2 ± |
| | | | 64.5 | (14.6- | 12200 | 407 | 12200 | 13900 | 1.14 | 2.14 | (1.0- | (168.0- | 24.6 |
| | | | | 46.4) | | | | | | | 1.0) | 168.0) | |
| | | Female | 722 ± | 20.2 | 28600 ± | 953 ± | 28600 ± | 32000 ± | 1.49 ± | 42.5 ± | 1.0 | 168.0 | 37.3 ± |
| | | | 84.3 | (9.5- | 19800 | 660 | 19800 | 25400 | 1.15 | 3.87 | (1.0- | (72.0- | 59.5 |
| | | | | 66.0) | | | | | | | 1.0) | 168.0) | |
| 100 | 22 | Male | 2700 ± | 57.1 | 170000 ± | 1700 ± | 170000 ± | 142000 | 0.734 | 56.9 | 1.0 | 168.0 | 577 |
| | | | 250 | | 84700 | 847 | 84700 | | | | | | |
| | | Female | 3660 ± | 183.9 | 302000 ± | 3020 ± | 302000 ± | 617000 ± | 0.166 ± | 40.4 ± | 1.0 | 168.0 | 1210 |
| | | | 215 | (126.3- | 8540 | 85.4 | 8540 | 109000 | 0.0316 | 4.47 | | | |
| | | | | 223.1) | | | | | | | | | |
| | 29 | Male | 2470 | 70.6 | 317000 | 3170 | 317000 | 317000 | 0.316 | 42.5 | 1.0 | 672.0 | 1.85 |
| | | Female | 3750 | 35.3 | 549000 | 5490 | 549000 | 549000 | 0.182 | 24.7 | 1.0 | 840.0 | 0.152 |

Data is presented as mean or individual values or mean ± SD for Cₘₐₓ, Cₗₐₛₜ, DNAUC, Cl, Vss and AUC values, and mean or individual values or median (range) for T_{1/2}, Tₘₐₓ and Tₗₐₛₜ
AUCₐₗₗ: AUC_{0-168 h} for Days 1 and 22 and AUC_{0-1008 h} for Day 29

TABLE 3
Summary of TK Parameters of HMBD-002-V4C26 Following Multiple Doses in ADA Negative Study Animals

| Dose | | | | | | DNAUCₐₗₗ | | | | | | | |
|---|---|---|---|---|---|---|---|---|---|---|---|---|---|
| (mg/kg/ | Study | | Cₘₐₓ | T_{1/2} | AUCₐₗₗ | ((h*ug/mL)/ | AUC₀₋ₗₐₛₜ | AUC_{0-inf} | CL | Vss | Tₘₐₓ | Tₗₐₛₜ | Cₗₐₛₜ |
| dose) | Day | Sex | (ug/mL) | (h) | (h*ug/mL) | (mg/kg/dose)) | (h*ug/mL) | (h*ug/mL) | (mL/h/kg) | (mL/kg) | (h) | (h) | (ug/mL) |
| 10 | 1 | Male | 246 | 65.95 | 14500 | 1450 | 14500 | 17300 | 0.5785 | 53.9 | 1.0 | 168.0 | 29.4 |
| | | Female | 258 ± | 59.6 | 13100 ± | 1310 ± | 13100 ± | 15100 ± | 0.666 ± | 55.0 ± | 1.0 | 168.0 | 23.9 ± |
| | | | 19.4 | (55.2- | 1210 | 121 | 1210 | 1470 | 0.0621 | 3.44 | (1.0- | (168.0- | 2.95 |
| | | | | 59.7) | | | | | | | 1.0) | 168.0) | |
| 30 | 1 | Male | 667 ± | 150.1 | 49700 ± | 1660 ± | 49700 ± | 91900 ± | 0.327 ± | 70.3 ± | 1.0 | 168.0 | 193 ± |
| | | | 67.9 | (147.9- | 1050 | 35.0 | 1050 | 2660 | 0.00946 | 1.63 | (1.0- | (168.0- | 5.51 |
| | | | | 155.3) | | | | | | | 1.0) | 168.0) | |
| | | Female | 786 | 110.7 | 54300 | 1810 | 54300 | 83900 | 0.358 | 57.0 | 1.0 | 168.0 | 185 |
| 100 | 1 | Male | 2090 ± | 177.3 | 164000 ± | 1640 ± | 164000 ± | 312000 ± | 0.334 ± | 71.3 ± | 1.0 | 168.0 | 675 ± |
| | | | 285 | (106.7- | 16400 | 164 | 16400 | 77600 | 0.0874 | 10.4 | (1.0- | (168.0- | 120 |
| | | | | 183.7) | | | | | | | 1.0) | 168.0) | |
| | | Female | 2230 ± | 150.1 | 183000 ± | 1830 ± | 183000 ± | 367000 ± | 0.279 ± | 61.3 ± | 1.0 | 168.0 | 799 ± |
| | | | 50.0 | (103.3- | 11300 | 113 | 11300 | 67100 | 0.0554 | 8.58 | (1.0- | (168.0- | 59.3 |
| | | | | 209.4) | | | | | | | 1.0) | 168.0) | |
| | 22 | Male | 4150 | 380.7 | 400000 | 4000 | 400000 | 1560000 | 0.0641 | 35.2 | 1.0 | 168.0 | 1960 |
| | | Female | 4200 | 137.3 | 314000 | 3140 | 314000 | 550000 | 0.183 | 36.05 | 1.0 | 168.0 | 1200 |
| | 29 | Male | 4050 | 285.1 | 1110000 | 11100 | 1110000 | 1210000 | 0.0829 | 31.7 | 1.0 | 1008.0 | 227 |
| | | Female | 3290 | 186.1 | 696000 | 6960 | 696000 | 717000 | 0.140 | 37.9 | 1.0 | 1008.0 | 77.8 |

Data is presented as mean or individual values or mean ± SD for Cₘₐₓ, Cₗₐₛₜ, DNAUC, Cl, Vss and AUC values, and mean or individual values or median (range) for T_{1/2}, Tₘₐₓ and Tₗₐₛₜ
AUCₐₗₗ: AUC_{0-168 h} for Days 1 and 22 and AUC_{0-1008 h} for Day 29

With dose increase from 10 to 100 mg/kg/dose, the systemic exposure (AUCₐₗₗ and/or Cₘₐₓ) to HMBD-002-V4C26 increased dose-proportionally in both sexes on Day 1 but increased more than dose proportionally in both sexes on Day 22 due to ADA presence and its influence on exposure with lower doses.

After repeated IV injection of HMBD-002-V4C26 at 10, 30, or 100 mg/kg/dose, no marked drug accumulation for HMBD-002-V4C26 was observed with AUC_{0-168 h} at 100 mg/kg/dose. However, due to ADA presence and its influence of exposure, a decreased systemic exposure was observed at 10 and 30 mg/kg/doses generally (Day 22/Day 1).

### 19.3 TRIAL OBJECTIVES

Primary objectives:

### Part 1—Dose Escalation Stage

The primary objective during the dose escalation stage is to identify the maximum tolerated dose (MTD), or the maximum tested dose, of HMBD-002 and to recommend doses for subsequent disease directed studies (i.e., recommended phase 2 dose [RP2D]).

### Part 2—Dose Expansion Stage

The primary objective during the expansion stage is to estimate the anti-cancer activity of HMBD-002 as a monotherapy at the RP2D in previously treated patients with triple negative breast cancer (TNBC), non-small cell lung cancer (NSCLC) and other VISTA-expressing malignancies.

### Secondary Objectives:

The secondary objectives are the following:
To characterize the pharmacokinetic (PK) properties of HMBD-002.
To further characterize the safety profile of HMBD-002 at the MTD or maximum tested dose.
To evaluate the incidence and persistence of anti-HMBD-002 anti-drug antibody (ADA) formation and its impact on the PK profile of HMBD-002.

### Exploratory Objectives:

The exploratory objectives are the following:
To evaluate the effects of treatment with HMBD-002 on the expression of VISTA and programmed death-ligand 1 (PD-L1) receptors in solid malignancy specimens before treatment, during treatment, and post-treatment, and correlate the results with anti-cancer activity.
To evaluate the quantitative and qualitative effects of HMBD-002 on various immunological effector cells, including myeloid derived suppressor cells (MDSCs) and T cells in blood and in tumor tissues.

### 19.4 STUDY ENDPOINTS

Safety endpoints include the following:
The incidence of dose limiting toxicities (DLTs) graded per the NCI CTCAE version 5.0 during the DLT assessment period;
The nature, frequency, and severity of AEs and SAEs, treatment discontinuations due to toxicity, and clinical laboratory abnormalities;
Physical examination and ECG findings;
VS measurements;
ECOG performance status scores.

Dose-finding endpoints include the MTD or maximum tested dose, and the RP2D.

Secondary endpoints:

Pharmacokinetic endpoints evaluated at various timepoints during HMBD-002 treatment include the following:
Maximum serum concentration (Cₘₐₓ);
Area under the serum concentration-time curve (AUC);
Elimination half-life (t_{1/2});
Clearance (CL);
Volume of distribution (V_{d});
ADA.

The antitumor activity of HMBD-002 will be measured by the following (see also Example 19.9):
Overall response rate (ORR) is defined as the proportion of patients achieving a best response of complete response (CR) or partial response (PR) according to RECIST v1.1.37.
Duration of response (DoR) is defined as the time from the date measurement criteria are first met for PR or CR to the date measurement criteria are first met for progressive disease (PD).
Duration of CR (DoCR) is defined as the time from the date when the measurement criteria are met for CR to the date measurement criteria are first met for PD.
Progression-free survival (PFS) is defined as the time from the date of initiation of study therapy to the date measurement criteria are first met for PD or death from any cause, whichever occurs first.
Progression-free survival at 6 months (PFS6) is defined as the percentage of patients alive and progression-free at 6 months (26 weeks) after the initiation of study therapy.
Overall survival (OS) is defined as the time from the date of initiation of study therapy to the date of death from any cause. Testing for ORR and PFS6 will be by one-sample binomial tests with a 1-sided type I error rate of 15%.

Distributions for PFS, DoR, DoCR, and OS will be estimated by Kaplan-Meier methodology. Efficacy parameters will be based on a modified intent-to-treat population which includes all patients who have received at least one full dose of any drug.

### 19.5 INVESTIGATIONAL DRUG PRODUCT

HMBD-002 is a humanized IgG4 mAb that blocks the interactions between the VISTA protein and its ligands. VISTA is, an immune checkpoint receptor that is expressed on both immune cells, predominantly in the myeloid compartment (i.e., myeloid derived suppressor cells) and a wide variety of cancer cells.

HMBD-002 is a mAb designed to bind with high affinity and specificity to human VISTA and block a predicted counter-structure binding site. The target residues are conserved between human, mouse, rat, and cynomolgus monkey. HMBD-002 was designed with a hIgG4 Fc domain to enable FcRn binding and increase antibody half-life in circulation. This design strategy allows HMBD-002 to avoid antibody-dependent cellular cytotoxicity (ADCC) and complement dependent cytotoxicity (CDC) activity due to the IgG4 construct's lack of affinity to FcγRIII and C1q proteins that mediate these immune effector functions. HMBD-002 shows high affinity, with Kd<1 nM towards human and cynomolgus monkey VISTA, and comparable binding to murine VISTA. Further, HMBD-002 exhibits highly specific VISTA binding based on binding comparison to other cell surface molecules in HEK293 expression systems and towards recombinant B7 family members.

HMBD-002 has been tested in various pre-clinical models for anti-tumor efficacy. In the syngeneic CT26 model of mouse colon adenocarcinoma this mAb demonstrated monotherapy anti-tumor activity by delaying tumor growth of subcutaneous flank implants and increasing survival. In the B16-BL6 syngeneic model of mouse melanoma, HMBD-002 showed activity only when combined with PD-1 blocking antibodies, whereas PD-1 blockade or VISTA blockade in isolation was not effective. These results suggest that VISTA and PD-1 checkpoints may be complementary in their ability to inhibit an immune response. HMBD-002 co-treatment reduced tumour MDSC infiltration, which may be one important means to increase anti-tumour immunity. Furthermore, in combination with an anti-PD-1 mAb using in vivo syngeneic cell line derived models of colorectal cancer, lymphoma, and melanoma, HMBD-002 substantially increased tumour growth inhibition over anti-PD-1 therapy alone.

HMBD-002 is supplied as a sterile, single use, preservative free solution intended for IV infusion in a vial containing 50 mg/mL. HMBD-002 is formulated with 20 mM Histidine, 8% (w/v) sucrose, 0.02% (w/v) polysorbate 80 (Tween 80) at pH 5.5.

The final container is a 4R USP Type I glass vial and contains 4 mL of HMBD-002.

The recommended storage condition for the drug product is −20±5° C. (−13 to 5° F.), protected from light. The compatibility of HMBD-002 with the infusion solution and the infusion set has been demonstrated. The study results indicate that HMBD-002 is compatible with the infusion solution and the infusion set that will be used at the clinical sites.

### 19.6 ADMINISTRATION, DOSES AND DOSAGE REGIME

### 19.6.1 Overall Design and Scheme

The planned study, HMBD-002-V4C26-01 is a Phase 1, FIH, open-label, multicenter, two-stage (Dose Escalation and Dose Expansion) study evaluating multiple doses and schedules of IV administered HMBD-002 in patients with advanced solid malignancies (i.e., locally advanced and unresectable, or metastatic) and no available therapeutics that can confer a reasonable likelihood of clinical benefit.

HMBD-002 is administered as a 60-minute IV infusion on Days 1, 8, and 15 of a 3-week treatment cycle (21-day cycle) as a monotherapy. If well-tolerated subsequent infusions of HMBD-002 can be administered over 30 minutes.

The study will be conducted in 2 stages: Part 1 (Dose Escalation) and Part 2 (Dose Expansion). In Part 1, increasing doses of HMBD-002 will be administered to characterize the safety, tolerability, PK and PD, and identify the maximum tolerated dose (MTD) or maximum tested dose, and the Recommended Phase 2 dose (RP2D).

The monotherapy starting dose regimen of HMBD-002 (i.e., the dose regimen in Cohort 1, Cycle 1) is 7 mg on Day 1 (33% [test] dose) and 20 mg on Days 8 and 15, followed by 20 mg on Days 1, 8, and 15 of a 3-week cycle. A treatment cycle is 21 days in duration, comprised of weekly×3 treatments with HMBD-002.

Patients may continue to receive study treatment(s) for up to 6 cycles if the patient agrees and is tolerating treatment, and there is potential clinical benefit. Study treatment will be discontinued for unacceptable toxicity, PD, voluntary study withdrawal by the patient, another discontinuation criterion is met, or study closure. The maximum number of cycles is set at 35.

In Part 1 (Dose Escalation), approximately 25-30 patients will be enrolled and treated with HBMD-002 as a monotherapy in a standard “3+3” design.

After the MTD and/or RP2D of HMBD-002 is established in Part 1 (the RP2D may be identical to the MTD but should reflect a dose that is tolerable over multiple cycles), Part 2 (Dose Expansion) of the study evaluates the preliminary anti-tumour activity of HMBD-002 in patients who have select malignancies, including advanced TNBC and NSCLC, and in patients with various solid malignancies. Part 2 will also further evaluate the safety, tolerability, PK, and pharmacodynamics of HMBD-002.

In Part 2 (Dose Expansion), up to 190 patients will be enrolled based on interim Go/No-Go decisions for 4 disease-directed expansion cohorts: TNBC (n=15 at interim) and NSCLC (n=15 at interim), each of which may be increased to up to 35 patients to estimate the anti-tumour activity of HMBD-002 more precisely if there is a sufficient signal in the interim analyses. There will also be one additional expansion cohort (n=50 each) for patients with other solid malignancies treated with HMBD-002.

Safety endpoints will be assessed during the study by documentation of DLTs, AEs and SAEs, treatment discontinuations due to toxicity, clinical laboratory abnormalities, physical examinations, ECG findings, VS measurements, and ECOG performance status scores. Assessment of anti-tumor activity will primarily focus on categorical objective response as defined according to the RECIST v1.1 (see Example 19.9).

### 19.6.2 Part 1: Dose Escalation

Dose escalation is initiated with single-agent HMBD-002. After at least 2 cohorts of patients treated with monotherapy HMBD-002 have been evaluated and requisite tolerability has been demonstrated, dose escalation may commence with HMBD-002 weekly×3.

In the dose escalation stage, HMBD-002 will be administered on Days 1, 8, and 15 of the first 21-day cycle. All safety data from all patients enrolled in a cohort is reviewed to confirm if any DLTs were experienced and determine whether enrollment into the next cohort can occur. In the dose escalation stage, a patient must have received all 3 of their scheduled HMBD-002 doses (Days 1, 8, and 15) during the DLT assessment period (Cycle 1) with follow-up through the end of this period to be eligible for DLT assessment, unless the patient experiences a DLT or drug-related toxicity resulting in the omitted treatment, in which case, the patient will have been assessable for DLT.

During the study, the PK and safety profiles of HMBD-002 may indicate that the agent is more conducive for administration on an alternate, albeit less intensive schedule such as a Day 1 and Day 8 every 3-week schedule or a Day 1 every 3-week schedule. If the HMBD-002 treatment schedule is modified to a Day 1 and 8 every 3-week or Day 1 every 3-week treatment schedule, a patient must have received both Day 1 and 8 doses of HMBD-002 in Cycle 1 (Day 1 and Day 8 every 3 week schedule) or a single dose of HMBD-002 on Day 1 (Day 1 every 3-week schedule), with follow-up through the end of the DLT assessment period (Day 21 of Cycle 1) to be eligible for DLT assessment, unless the patient experiences a DLT or drug-related toxicity, resulting in the omitted treatment.

If a DLT necessitates enrollment of additional patients into a cohort, all safety data for that cohort will be reviewed after those additional patients have completed the DLT assessment period. Based on evaluation of the data, it may be decided that enrollment at an intermediate HMBD-002 dose level not specified in this protocol will be more conservative than specified, and the next higher dose will be an intermediate dose.

If 0 of at least 3 patients or ≤1 of at least 6 patients within a cohort experience a DLT, then enrollment in the next higher dosing cohort may commence with consensus of the Safety Review Committee (SRC). The SRC will consist of at least three members including Hummingbird Bioscience's Chief Medical Officer, or designee, the Medical Monitor, or designee, and at least one of the Principal Investigators. The committee will review safety data from the current cohort and previous cohorts before deciding on dose escalation for the next dose-level cohort. Dose escalation may take place only after each patient in any given cohort has reached Day 21 of Cycle 1, and the SRC has agreed that dose escalation may occur.

### 19.6.3 Dose Escalation Process

Given that there is a potential for cytokine release syndrome (CRS) due to the mechanism of action of HMBD-002, and the occurrence of CRS with other unrelated VISTA mAbs, the following measures will be taken during dose escalation of HMBD-002:
The first dose administered in Cohort 1 will be a “step” or “test dose” at 33% of the full dose on Day 1 of Cycle 1. For each subsequent dose level (e.g., “X mg” dose level), all patients will receive 50% of the dose as a “step” or a “test dose” on Day 1 of Cycle 1. Patients should be observed in the clinic for at least 6 hours after receiving their first (“step” or “test’) dose. There should be a follow up visit to the clinic within 24 hours.
For the first full dose (X mg), one week later, on Day 8 of Cycle 1, patients should be hospitalized for approximately 24 hours after receiving treatment and should be assessed as an outpatient at 48 hours (Day 9) and 72 hours (Day 10) after treatment.
For the second full dose (X mg) on Day 15 of Cycle 1, patients should be observed in the clinic for at least 6 hours after treatment.
For all subsequent doses during Cycle 2+, patient should receive the full dose and be observed for at least 1 hour after treatment.
Patients experiencing neurotoxicity of any Grade, Grade 2 CRS/infusion-related reactions (IRR) for any additional dose infusions should be adequately monitored for symptom resolution in the hospital. Any patient experiencing Grade 3 CRS/IRR or Grade 3 neurotoxicity should be discontinued from study.
If any Grade 3 CRS/IRR is observed, all further patients accrued to the study will receive dexamethasone 20 mg orally or IV 6 and 12 hours before treatment with HMBD-002.
Patients may be treated with the following pre-medications based on occurrence of Grade 1-2 fever, rigors, myalgia/arthralgia and/or relevant AEs during a prior treatment: diphenhydramine 25-50 mg IV/po and/or acetaminophen within 30-60 minutes prior to HMBD-002 treatment. If such AEs are observed to be common, this premedication regimen may be mandated for all patients.

At least 3 patients will receive treatment with HMBD-002 at the starting dose level (20 mg) in the assessment of single-agent HMBD-002. A 3+3 design will be utilized. The first patient enrolled into each new dose escalation cohort must complete the full Cycle 1 before accrual of additional patients into the cohort. During dose escalation, the first 3 patients in each new dose level must have staggered treatment by at least 7 days.

The following general dose escalation scheme will be followed:
In escalating doses of HMBD-002, at least 3 patients must be treated at the starting dose level of 20 mg, with each of the three patients receiving a 7 mg “test dose” on Day 1 of Cycle 1.
If none of the first 3 DLT-assessable patients in a dosing cohort experiences a DLT, enrollment of the next higher dose cohort may commence, with all new patients in each cohort receiving a 50% test dose on Day 1 of Cycle 1.
If a single DLT-assessable patient in a cohort experiences DLT, then up to 6 total safety-assessable patients are to be enrolled at that dose level. If no more than 1 of the 6 patients experiences a DLT, then enrollment in the next higher dose cohort may commence.
Enrollment in the next higher dose cohort can commence only when the last patient enrolled in the current dose cohort completes the DLT assessment period, if there is no DLT in any of the first 3 patients or no more than 1 DLT in the first 6 new patients.
If a second DLT occurs in any of the first 6 patients in a cohort, then the MTD will have been exceeded and the previous lower dose level will be considered the MTD, provided that DLT has not occurred in more than 1 of the first 6 DLT-assessable patients.
Cycle 2 treatment may be held for up to 21 days following the DLT observation period for toxicity or any other reasons, after which time the patient must be discontinued from treatment.
Up to 15 patients may be treated at the MTD (or maximum tested dose if no MTD is identified), or dose below the MTD if there is evidence suggesting a more favorable risk/benefit profile) to provide further characterization of the safety, tolerability, PK, and pharmacodynamics of HMBD-002. The 50% test dose will not be required in these patients.

Although decisions regarding dose escalation will be made based on review of data from the DLT assessment period, safety data will also be collected from all patients continuing treatment and this will be reviewed periodically. Any detected cumulative toxicity may require later dose reductions, dose-schedule changes, or other action as appropriate, including further refinement of the MTD.

In situations in which DLTs have occurred that would prevent further dose escalation, and these DLTs are severe but not life-threatening (i.e., persistent Grade 3 fever, malaise), re-evaluation of specific dose cohorts may be considered with co-administration of appropriate supportive care agents (including, albeit not limited to, hematopoietic growth factors, corticosteroids, anti-depressants) concomitant with initial HMBD-002 therapy after discussion with the medical monitor.

Dose modifications of HMBD-002 are not permitted during Cycle 1 (DLT assessment period) of the dose escalation stage of the study. Patients who experience DLT during Cycle 1 may be continued on study at a reduced dose if the dose has been demonstrated to be safe during the dose escalation process and after discussion with the Medical Monitor. The discussion will be documented by the Medical Monitor and Study Coordinator. Following the DLT assessment period, doses may be held or modified for specific toxicities and/or laboratory abnormalities in Cycles 2+ for patients participating in the dose escalation stage and in all Cycles for patients in the dose expansion stage.

The treatment of each of the first three patients in each new cohort during the dose escalation stage must be staggered by 7 days before accrual of additional patients into the cohort. The dose escalation will occur according to the simplified schema presented in Table 4.

TABLE 4
Dose Escalation Schema

| If | Then |
|---|---|
| 0/3 patients without DLT at | Escalate to dose level X + 1 |
| dose level X in the DLT assessment | |
| period or ≤1/6 patients with DLT | |
| 1/3 patients with DLT | Evaluate 6 patients at dose level X |
| ≥2/6 patients with DLT | Dose level X − 1 is MTD ^{a} |

Abbreviations:
DLT, dose limiting toxicity;
MTD, maximum tolerated dose
^{a} This dose will be considered the MTD if no more than 1 patient experiences a DLT, provided at least 6 new patients have received treatment at this dose level and are DLT-assessable. The dose level will be defined as the 50% test dose (50% X) on Day 1 of Cycle 1 with all other doses at the full dose (X)

### 19.6.4 Dose Escalation Applicable to HMBD-002

Regarding dose escalation of HMBD-002, based on the results from Good Laboratory Practice (GLP) toxicology studies, Cohort 1 patients will receive HMBD-002 at the starting dose of 7 mg on Day 1 and 20 mg on Days 8, and 15 of the first 21-day cycle (Cycle 1). A dose of 20 mg will be administered on Days 1, 8, and 15 of all subsequent cycles. The maximum dose of HMBD-002 for the next planned cohort is dependent on the safety findings from the current cohort as presented in Table 5.

TABLE 5
Maximum Dose Beyond Starting Dose

| If | Then |
|---|---|
| 0 patients with no AE >Grade 1 | Next cohort will receive dose |
| at dose level X (≤80 mg) ^{a} | level ≤3.0 × current dose |
| 0 patients with no AE >Grade 1 | Next cohort will receive dose |
| at dose level X (>80 mg) ^{a} | level ≤2.0 × current dose |
| ≥1 patient experience Grade 2 | Next cohort will receive dose |
| non-DLT AE (≤80 mg) | level ≤2.0 × current dose |
| ≥1 patient experience Grade 2 | Next cohort will receive dose |
| non-DLT AE (>80 mg) | level ≤1.5 × current dose |
| ≥1 patient experience ≥Grade | Next cohort will receive dose |
| 3 non-DLT | level ≤1.5 × current dose |
| 1 of 6 patients experiences DLT | Next cohort will receive dose |
| | level ≤1.33 × current dose |
| ≥2 patient experiences any | Next cohort (Cohort −1) will receive |
| grade DLT ^{b} in Cohort 1 | dose level 50% reduced from Cohort 1: |
| | 0.45 mg/m² |
| ≥2 patients experience any | Next cohort (Cohort −2) will receive |
| grade DLT ^{b} in Cohort −1 | dose level 33% reduced from |
| | Cohort −1: 0.30 mg/m² |

Abbreviations:
AE = adverse event;
DLT = dose limiting toxicity
^{a} Dose level X refers to the current dose level.
^{b} DLTs must be events that cannot be clearly attributed to a cause other than study drug.

The maximum dose of HMBD-002 for the next planned cohort is dependent on the safety findings in the current cohort, as follows. Note that “X” is the mg dose evaluated in the prior cohort:
If no patient experiences a Grade >1 HMBD-002-related AE (any Grade >1 AE that cannot be clearly attributed to an extraneous cause [e.g., cancer, PD]), and the current dose level is $80 mg: ≤3.0 (X) mg.
If no patient experiences a Grade >1 HMBD-002-related AE (any Grade >1 AE that cannot be clearly attributed to an extraneous cause [e.g., cancer, PD]), and the current dose level is >80 mg: ≤2.0 (X) mg.
In cases where there has not been a DLT, if at least 1 patient experiences a Grade 2 HMBD-002-related AE, and the current dose level is ≤80 mg: ≤2.0 (X) mg.
In cases where there has not been a DLT, if at least 1 patient experiences a Grade 2 HMBD-002-related AE, and the current dose level is >80 mg: ≤1.5 (X) mg.
In cases where there has not been a DLT, if at least 1 patient experiences a Grade 3 HMBD-002-related non-DLT: ≤1.5 (X) mg.
If 1 of 6 patients experiences a DLT: ≤1.33 (X) mg.
Lower dose increases (i.e., 25% increase) may be considered based on the frequency and severity of non-DLT AEs.
Following 25% or 33% dose escalations, a higher dose escalation increment (up to a 50% increase) may be re-instituted if no Grade ≥3 HMBD-002-related non-DLT AEs occur in the 2 subsequent (consecutive) cohorts.
If a DLT occurs in 2 patients treated in Cohort 1, the subsequent cohort (Cohort-1) will receive HMBD-002 at a dose reduced by 50% from Cohort 1 (Cohort-1=10 mg with 3.3 mg as the administered dose on day 1 of Cycle 1).
If a DLT occurs in 2 patients treated in Cohort-1, the subsequent cohort (Cohort-2) will receive HMBD-002 at a dose reduced by ˜33% from Cohort-1: (Cohort-2=7 mg with 2.2 mg as the administered dose on day 1 of Cycle 1).
In a circumstance in which the dose intensity on the Days 1, 8, 15 (weekly) every 3-week schedule is too high, and there is support for a less dose intensive (e.g., Days 1 and 8 every 3-week or Day 1 every 3-week) schedule based on clinical safety and PK data, a new patient cohort using a Days 1 and 8 every 3-week or a Day 1 every 3-week schedule can be opened for accrual of new patients.
   Since a weekly×2 (Days 1 and 8) every 3-week schedule represents a 33% reduction and a Day 1 every 3-week schedule represents a 66% reduction in HMBD-002 dose intensity compared to the Day 1, 8, and 15 every 3-week schedule, the first patient cohort is to receive the same HMBD-002 dose as determined to be safe (i.e., MTD or less) on the Day 1, 8, 15 every 3-week schedule. Dose escalation will be performed according to the scheme described for the Day 1, 8, and 15 every 3-week schedule. This includes a 50% test dose on Day 1 of Cycle 1.
Additional patients, particularly those who are amenable to and consent to successive tumor biopsies (biopsies in which the procedures do not confer a significant absolute risk of mortality or major morbidity in the patient's clinical setting and at the institution completing the procedure is estimated to be 2% or higher), may be treated at dose levels which are fully assessable for DLTs and have been cleared with respect to dosing at the next higher dose level.
If DLT necessitates enrollment of additional patients into a cohort, all safety data for that cohort will be reviewed after the additional patients have completed the DLT assessment period. Based on evaluation of the data, the SRC and/or Sponsor may decide to enroll patients at an intermediate HMBD-002 dose level that is not specified in this protocol but that will be between the planned dose levels.
Cycle 2 treatment may be held for up to 21 days following the DLT observation period for toxicity or any other reasons, after which time the patient must be discontinued from treatment

HMBD-002 may be evaluated for its safety and efficacy in combination therapy, e.g. in combination with anti-PD-1 or anti-PD-L1 therapy.

### 19.6.5 Part 2: Dose Expansion

Following determination of the MTD/RP2D level of HMBD-002, which may represent the MTD or a lower dose based on factors such as tolerance of repetitive cycles (i.e., evidence of cumulative toxicity), the results of PK studies (i.e., a dose level exceeding PK parameters indicative of biological activity), the results of pharmacodynamic studies (i.e., saturation of a target pharmacodynamic effect), and/or anticancer activity, separate disease-defined cohorts of patients are treated with HMBD-002.

For all patients enrolled in the expansion stage, tumor biopsies will be requested pretreatment, near the end of Cycle 2 or at the time of PD, if earlier. The tumor biopsy procedure must not pose a significant risk as defined as an associated absolute risk of mortality or major morbidity in the patient's clinical setting and at the institution completing the procedure is estimated to be 2% or higher. Demonstration of VISTA+ immunohistochemistry (IHC) is not required before beginning treatment.

The following distinct dose-expansion cohorts will be evaluated:
TNBC-15 previously treated patients with advanced TNBC treated with HMBD-002 as a monotherapy, with the possibility to expand further if a sufficiently positive signal is observed.
NSCLC-15 previously treated patients with advanced NSCLC treated with HMBD-002 as a monotherapy, with the possibility to expand further if a sufficiently positive signal is observed.
50 patients with a variety of malignancies amenable to serial biopsy (i.e., the biopsy procurement is not a significant risk procedure (i.e., ≥2% estimated major morbidity or mortality in the patient's clinical setting and at the institution completing the procedure) treated with HMBD-002.

Up to 190 patients may be treated in the Dose Expansion Stage. Any of the expansion cohorts may be expanded to 35 patients each if a sufficient level of activity (>1 objective response) is noted at the first interim assessment (15 patients).

Doses may be modified in response to treatment-related toxicity.

### 19.6.6 Definition of Dose-Limiting Toxicity

The DLT assessment period is 21 days (Cycle=21 days). Patients enrolled in Part 1 (Dose Escalation) and are receiving HMBD-002 monotherapy must receive all 3 of their scheduled HMBD-002 doses (Days 1, 8, and 15) during the DLT assessment period with completed follow-up data available through 21 days to be eligible for DLT assessment (i.e., DLT-assessable). Patients who experience a DLT or other drug-related toxicity resulting in omitted treatment during the DLT assessment period will also be eligible for DLT assessment.

Toxicities will be assessed by the Investigator using the National Cancer Institute (NCI) Common Terminology Criteria for Adverse Events (CTCAE), version 5.0. The relationship of an AE to HMBD-002 (i.e., attribution) is to be assessed.

A DLT is defined as any of the following AEs occurring during the DLT assessment period, regardless of investigator attribution to HMBD-002, unless the AE can be clearly and incontrovertibly attributed to an extraneous cause (e.g., cancer, PD).

### Hematologic DLT:

Grade 3 or 4 febrile neutropenia
   defined as an absolute neutrophil count (ANC)<1000/mm³ with a single temperature of >38.3° C. (101° F.) or a sustained temperature of ≥38° C. (100.4° F.) for more than 1 hour with or without life-threatening consequences and urgent intervention indicated.
Grade 4 neutropenia (ANC <500/μL) lasting >7 consecutive days (complete blood count [CBC] plus differential must be measured every 2 to 3 days until recovery of ANC to Grade ≤1).
Grade 4 thrombocytopenia (platelets <25,000/μL); or Grade 3 thrombocytopenia (platelets <50,000/μL) lasting >7 days in patients without demonstrable bone marrow involvement by tumor; or Grade 3 or 4 thrombocytopenia associated with clinically significant bleeding.

### Nonhematologic DLT:

Any nonhematologic AE ≥Grade 3 according to the NCI CTCAE, version 5.0:
   An exception is nausea, vomiting, diarrhea, electrolyte/metabolic/glucose abnormalities, constipation, fever, or fatigue, in which there has been suboptimal prophylaxis and management and that resolves to Grade ≤1 within 72 hours.
   An exception is Grade 3 elevation in aspartate aminotransferase (AST) and/or alanine aminotransferase (ALT) that do not meet Hy's law. Grade 3 AST and/or ALT elevations that meet Hy's law require the addition of total bilirubin >2× upper limit of normal (ULN) in the absence of findings of cholestasis (absence of elevation of alkaline phosphatase to >2×ULN) AND no other reason can be found to explain the combination of increased ALT/AST and total bilirubin. Grade 3 transaminase elevations that are exceptions from Hy's law require resolutions to $ Grade 1 within 7 days, provided that Hy's law criteria are also not met.
Grade 3 manifestations of CRS/immune effector cell-associated neurotoxicity syndrome (ICANS), as defined by the NCI CTCAE version 5.0. Such patients who experience Grade 3 or 4 CRS or ICANS due to treatment with HMBD-002 should be discontinued from study.
Grade 3 IRR or Grade 3 immune related toxicity. Such patients should be discontinued from study.

### Both Hematologic and Non-Hematologic DLT:

Any possible drug-related toxicity resulting in the omission of the Day 8 or 15 dose during Cycle 1, or Day 8, if the schedule is modified to a Day 1 and 8 every 3-week schedule.
Any toxicity that delays the administration of Cycle 2 by >21 days because it has failed to meet recovery criteria.
Any treatment-related toxicity that causes treatment discontinuation during Cycle 1.
Any Grade 5 toxicity.
Any other significant toxicity considered by the Investigator and Sponsor's medical representatives to be a DLT.

Treatment-emergent AEs through 30 days after last dose will be summarized by MedDRA™ Version 13.1 (or higher) System Organ Class and preferred term. The incidences and percentages of patients experiencing each AE preferred term will be summarized with descriptive statistics. AEs will also be summarized by NCI-CTCAE, v5 grade and by causality (attribution to study treatment). DLTs, Grade 3-4 AEs, SAEs, and AEs resulting in dose modification or treatment discontinuation will also be summarized by preferred term.

### 19.6.8 Criteria for Dosing and Retreatment

Within a treatment cycle, HMBD-002 should be withheld on any scheduled treatment day if, during the past 24 hours, the patient experiences any of the following events:
a. ≥Grade 2 diarrhea, despite optimal anti-diarrheal therapy
b. ≥Grade 2 vomiting, despite optimal anti-emetic therapy
c. ≥Grade 3 fatigue that is not relieved by rest and limits self-care activities of daily living
d. ≥Grade 3 transaminase elevation
e. Platelet count <50×10⁹/L
f. Neutrophil count <0.5×10⁹/L

To commence treatment in Cycle 2 and beyond, patients must meet the following criteria:
a. ANC ≥1.00×10⁹/L
b. Hemoglobin ≥8 mg/dl
c. Platelet count ≥75×10⁹/L
d. Calculated creatinine clearance >35 mL/min
e. Total bilirubin≤1.8× institutional ULN
f. AST and ALT $4× institutional ULN or ALT and AST ≤5× institutional ULN if the patient has hepatic metastases.

Dose modifications of study treatment (HMBD-002) within a treatment cycle are not permitted during Cycle 1 (DLT assessment period) of Part 1 (Dose Escalation). In the circumstance that a patient has experienced an event that qualifies as a DLT a patient may restart study treatment at the same or at a lower dose.

Dose modifications may be made based on transaminase elevations, thrombocytopenia, and/or neutropenia.

Upon injection of HMBD-002, it is possible that patients may experience transient fever, chills, nausea, fatigue, vomiting, headache, and increased liver enzymes. They may also have manifestations of CRS including fever, rigors, malaise, fatigue, anorexia, myalgias, arthralgias, nausea, vomiting, headache, diarrhoea, tachycardia, hypotension, change in cardiac output, widened pulse pressure, rash, tachypnea, elevated D-dimer, transaminitis, azotemia, hypoxemia, hypofibrino-genemia±bleeding, hyperbili-rubinemia, mental status changes, confusion, delirium, aphasia, hallucinations, tremor, dysmetria, altered gait, and/or seizures. CRS can be graded according to the NCI CTCAE Version 5 criteria.

Mild CRS: Mild (Grade 1, fever with or without constitutional symptoms) early reactions generally do not require intervention. Acetaminophen or similar anti-inflammatory drugs (nonsteroidal anti-inflammatory drugs; NSAIDs) can be administered to treat fever and associated constitutional manifestations, if needed. For possible severe AEs due to CRS, interventions may include blood pressure support, and treatment with corticosteroids, the anti-interleukin-6 (IL6) mAb tocilizumab, and/or management of hypersensitivity manifestations.

Moderately severe CRS: In case of CRS Grade 2 or higher, tocilizumab with or without corticosteroids may be administered to treat the various relevant and potentially serious manifestations of CRS. The recommended dose of tocilizumab is 12 mg/kg IV for patients who weigh less than 30 kg and 8 mg/kg for patients who weight 30 kg or above. The agent should be administered as an IV infusion over 60 minutes. Tocilizumab may be readministered if clinical improvement does not occur within 24 to 48 hours. CRS Grade ≥2 or higher should be confirmed by measuring IL6, CRP, and ferritin at baseline (schedule labs) and at the time of the event and before discharge. For subsequent treatment, if indicated, pre-medication with acetaminophen, nonsteroidal anti-inflammatory agents are allowed. Low-dose corticosteroids may be used as premedication to reduce the severity of reactions if such arise to HMBD-002, but only after discussion with the Medical Monitor.

Severe CRS (grade 3): For possible severe AEs due to CRS, intervention may include blood pressure support, and treatment with corticosteroids, the anti-interleukin-6 (IL6) mAb tocilizumab (see above), and/or management of hypersensitivity manifestations.

Standard supportive care for CRS may be administered.

AEs associated with HMBD-002 may represent an immune-related response (irAE). Severe and life-threatening irAEs should be treated with IV corticosteroids followed by oral steroids, e.g. initial dose of 1 to 2 mg/kg prednisone or equivalent. Other immunosuppressive treatment should begin if the irAEs are not controlled by corticosteroids. The corticosteroid taper should begin when the irAE is ≤Grade 1 and continue at least 4 weeks. Additional appropriate medical therapy may include but is not limited to: Epinephrine, IV fluids, Antihistamines, NSAIDs, Acetaminophen, Narcotics, Oxygen, Pressors, and/or Corticosteroids.

### 19.6.9 Prior and Concomitant Medications

### Allowed Medications/Therapies

Outside of the DLT assessment period, in the setting of thrombocytopenia or neutropenia, administration of thrombopoietic agents (i.e., romiplostim, eltrombopag) or granulocyte colony stimulating factor (G-CSF) agents (i.e., filgrastim, peg filgrastim) is permitted in accordance with institutional or American Society of Hematology (ASH)/ASCO guidelines. Erythropoietin analogs are also permitted in accordance with the above-mentioned guidelines. These agents should not be employed during the DLT assessment period unless severe cytopenia consistent with DLT are identified. HMBD-002 treatment should be suspended until the G-CSF agent has been stopped if used for amelioration of neutropenia during the DLT assessment period.

Comprehensive supportive care is permitted during the study, including but not limited to anti-emetic and anti-diarrheal agents, appetite stimulants, stimulants (i.e., modafinil), anti-cachexia therapy (i.e., fish-oil supplements), anti-depressants, opiate and non-opiate analgesics, antibiotics, selective use of corticosteroids, and platelet/neutrophil growth factors as indicated above.

Patients who enter the study receiving ongoing bisphosphonate therapy or anti-receptor activator of nuclear factor kappa β ligand therapy (e.g., denosumab) may continue to receive these agents during study participation. Patients with resistant prostate cancer who are receiving gonadotrophin releasing hormone (GnRH) agonists as medical castration should continue receiving GnRH agonists during the study. Patients with advanced prostate cancer who are receiving LHRH agonists are permitted onto the study and should continue use of these agents during study treatment.

### Prohibited Medications/Therapies

Enrolled patients may not receive investigational or approved anticancer agents including cytotoxic chemotherapy agents, anticancer tyrosine kinase inhibitors, immunotherapy, or therapeutic mAbs, except as directed per this protocol (e.g. an anti-PD-1/PD-L-1 antibody). Palliative radiation is not permitted during study enrollment unless it is being performed for an existing, nonprogressive metastasis/symptoms and involves a narrow radiation port (e.g., solitary bone lesion). Live or live attenuated vaccines are not permitted within 30 days prior to the first dose of study treatment and while participating in the study. Systemic glucocorticoids are permitted only for the following purposes: modulate symptoms of an AE that is suspected to have an immunologic etiology, as needed for the prevention of emesis, premedication for IV contrast allergies, short-term oral or IV use in doses >10 mg/day prednisone equivalent for COPD exacerbations, or for chronic systemic replacement not to exceed 10 mg/day prednisone equivalent.

Patients should not be treated with continuous systemic steroids at doses higher than the equivalent of prednisone 10 mg daily. If continuous systemic steroid therapy at doses higher than the equivalent of prednisone 10 mg daily is required, HMBD-002 should not be administered untilE steroids have been tapered back to baseline.

### 19.7 PATIENT POPULATION

During the dose escalation stage, approximately 40-50 patients are treated. This estimate includes 25-30 and 15-20 patients with solid malignancies that are relapsed/refractory to currently available therapies treated with HMBD-002.

The expansion stage includes the treatment of 15-30 patients each with triple negative breast cancer (TNBC) and non-small cell lung cancer (NSCLC) treated with HMBD-002 as a monotherapy (up to 35 patients), and up to 50 patients with other malignancies treated with HMBD-002. If sufficient activity is observed in any NSCLC or TNBC cohort, the cohort may be expanded to at least 35 patients.

All patients must have a pre-treatment (prior to study therapy dosing) imaging (computed tomography [CT]) scan of the chest/abdomen/pelvis or magnetic resonance imaging (MRI), if indicated; and a tumor biopsy is requested (if the patient is to be enrolled in the expansion stage) within 21 days prior to their first study therapy dose. There may be circumstances in which patients are enrolled without undergoing successive tumor biopsies only if permitted to do so by the medical monitor. Patients with skin, subcutaneous or lymph node metastases may also have tumor evaluations (including measurements, with a ruler) by means of physical examination. Tumor measurements and disease response assessments are also to be performed at the end of Cycle 2, and then approximately every 2 cycles thereafter, until development of PD. Tumor measurements and disease response assessments are also to be performed at the EOT visit. Additionally, measurement of tumor markers is strongly encouraged in situations in which the tumor marker is employed in the clinical management of patients with a given malignancy, and the patient has a known elevation of a given marker. All patients enrolled in HMBD-002 doses near or at the MTD in the dose escalation stage, and all patients enrolled in the expansion stages, will be requested to have serial tumor biopsies performed prior to Cycle 1 treatment and at the end of Cycle 2 or at the time of PD, if earlier, as long as the patient will not be subjected to significant risk in which the procedure associated absolute risk of mortality or major morbidity in the patient's clinical setting and at the institution completing the procedure is estimated to be 2% or higher. Otherwise, an archival tumor specimen should be submitted.

### 19.7.1 Inclusion Criteria

1. The patient must have histologic or cytologic evidence of a malignant solid cancer (any histology) and must have advanced or metastatic disease with no available therapies known to confer clinical benefit.
2. Patients enrolled in the dose escalation stages must have disease that is resistant to or relapsed following available standard systemic therapy, or for which there is no standard systemic therapy or reasonable therapy in the physician's judgment likely to result in clinical benefit or if such therapy has been refused by the patient. Documentation of the reason must be provided for patients who have not received a standard therapy likely to result in clinical benefit.
3. Tumor tissue (a minimum of 10 and up to 15 unstained slides), or paraffin block, ideally from the patient's most recent biopsy, must be located with plans to be forwarded to the study center or made available prior to the first dose of study therapy. A fresh tumor biopsy is preferable to using archival samples and will be obtained if archival samples are not available as long as the biopsy procedure is not a significant risk procedure defined as one without mortality or major morbidity in the patient's clinical setting and at the institution completing the procedure is estimated to be 2% or higher. The medical monitor must be contacted to review documentation of biopsy risk and waive the requirement for fresh biopsy.
   a. Patients enrolled in the expansion stages will be requested to undergo a tumor biopsy during the screening period and at the end of Cycle 2 (Days 16-21; ±2 days) or at the time of PD, if earlier, if the procedure is not a significant risk procedure as defined above.
4. The patient must have disease that is measurable by Response Evaluation Criteria in Solid Tumors (RECIST), version 1.1 (APPENDIX A). For patients with prior radiation therapy, measurable lesions must be outside of any prior radiation field(s), unless disease progression has been documented at that site after radiation.
5. The patient is ≥18 years old on the day of signing the consent form.
6. The patient has an ECOG PS of ≤1.
7. The patient has adequate baseline organ function, as demonstrated by the following:
   a. Creatinine clearance (CrCL) ≥30 mL/min as calculated by the Cockcroft-Gault formula:

$$\begin{matrix}i . CrCl ( male ) = ( [ 140 - age ] \times weight in kg ) / ( serum creatinine \times 72 ) \\ ii . CrCl ( female ) = CrCl ( male ) \times 0.85\end{matrix}$$

b. Bilirubin ≤1.5× institutional ULN.
c. AST and ALT ≤2.5× institutional ULN. Patients may have ALT and AST <5× institutional ULN if the patient has hepatic metastases.
8. For patients not taking warfarin or other oral anticoagulants: international normalized ratio (INR) ≤1.5 or prothrombin time (PT)≤1.5×ULN; and either partial thromboplastin time or activated partial thromboplastin time (PTT or aPTT)≤1.5×ULN. Patients taking warfarin should be on a stable dose that results in a stable INR <3.5. Among patients receiving other oral anticoagulant therapy, PT or aPTT must be within the intended therapeutic range of the anticoagulant and/or SC therapies should be monitored as standard per institution.
9. The patient has adequate baseline hematologic function, as demonstrated by the following:
   a. ANC ≥1.5×10⁹/L.
   b. Hemoglobin ≥9 g/dl and no red blood cell (RBC) transfusions during the prior 14 days.
   c. Platelet count ≥100×10⁹/L and no platelet transfusions during the prior 14 days.
10. The patient has a left ventricular ejection fraction (LVEF) ≥45% as determined by either echocardiography (ECHO) or multi-gated acquisition (MUGA) scanning.
11. A female participant is eligible to participate if she is not pregnant, not breastfeeding, and at least one of the following conditions applies:
   a. Not a woman of childbearing potential (WOCBP).
   b. A WOCBP who agrees to follow contraceptive guidance during the treatment period and for at least 120 days after the last dose of study treatment.
   c. If the patient is a WOCBP, she has had a negative serum or urine pregnancy test within 72 hours prior to treatment.
12. A male patient is eligible to participate if he agrees to follow contraceptive guidance during the study period and for at least 120 days after the last dose of study treatment.

### 19.7.2 Inclusion Criteria for HMBD-002 Expansion Cohorts:

### TNBC

1. The patient must have histologic or cytologic evidence of TNBC that is metastatic. TNBC is defined by clinician's assessment and can include human epidermal growth factor receptor 2 (HER2) negative by 2018 American Society of Clinical Oncology (ASCO)/College of American Pathologists (CAP) criteria, estrogen receptor (ER)≤10% and progesterone receptor (PR)≤ 10%.
2. Although documentation of a VISTA IHC+ tumor is not required to begin treatment, the patient will have a tumor biopsy during the screening period and at the end of Cycle 2 (Days 16-21; ±2 days) or at the time of PD, if earlier, if the procedure is not a significant risk procedure as defined as one for which the procedure associated absolute risk of mortality or major morbidity in the patient's clinical setting and at the institution completing the procedure is estimated to be 2% or higher. Otherwise, the patient will submit an archival tumor specimen.
3. The patient must have received appropriate treatment with at least one prior regimen for TNBC and no available therapies known to confer clinical benefit.

### NSCLC

1. The patient must have histologic or cytologic evidence of NSCLC that is advanced disease, defined as cancer that is either metastatic or locally advanced and unresectable.
2. Although documentation of a VISTA IHC+ tumor is not required to begin treatment, the patient will have a tumor biopsy during the screening period and at the end of Cycle 2 (Days 16-21; ±2 days) or at the time of PD, if earlier, if the procedure is not a significant risk procedure as defined as one for which the procedure associated absolute risk of mortality or major morbidity in the patient's clinical setting and at the institution completing the procedure is estimated to be 2% or higher. Otherwise, the patient will submit an archival tumor specimen.
   3. The patient must not have an activating mutation of the epidermal growth factor receptor (EGFR) or anaplastic lymphoma kinase (ALK).
   4. The patient should have received treatment with FDA approved targeted therapy if they harbor other genomic aberrations for which FDA-approved targeted therapy is available (e.g., ROS rearrangements, BRAF V600E mutation, met exon 14 skipping mutation), but such patients who have not received prior treatment are eligible if they have been given informed consent as to the improvements in overall survival that have been demonstrated with such treatment.
   5. The patient must have had disease progression on at least one FDA-approved or comparable standard therapy for NSCLC.
   6. The patient should have received appropriate prior treatment with a mAb to programmed death receptor-1 (PD-1) or PD-L1, but such patients who have not received prior treatment are eligible for HMBD-002 monotherapy as long as they have given proper informed consent (informed consent form) informing them that improvement in overall survival has been demonstrated with such treatment.

### Multiple Other Cancers

1. The patient must have histologic or cytologic evidence of an advanced, unresectable or metastatic cancer aside from TNBC and NSCLC with no available therapies known to confer clinical benefit.
2. Although documentation of a VISTA IHC+ tumour is not required to begin treatment, the patient will have a tumour biopsy during the screening period and at the end of Cycle 2 (Days 16-21; ±2 days) or at the time of PD, if earlier, if the procedure is not a significant risk procedure as defined as one for which the procedure associated absolute risk of mortality or major morbidity in the patient's clinical setting and at the institution completing the procedure is estimated to be 2% or higher. Otherwise, the patient will submit an archival tumour specimen.
3. The patient must have had appropriate treatment for their cancer and there is no available therapy available.

### 19.7.3 Exclusion Criteria

1. The patient has received prior therapy with an anti-PD-1, anti-PD-L1, or anti PD L2 agent or with an agent directed to another stimulatory or co-inhibitory T-cell receptor (e.g., CTLA-4, OX 40, CD137), and was discontinued from that treatment due to a Grade 3 or higher immune-related adverse event (irAE). This does not apply to irAEs due to prior treatment with cell therapy.
2. The patient has received prior radiotherapy within 2 weeks of treatment. Participants must have recovered from all radiation-related toxicities, not require corticosteroids, and not have had radiation pneumonitis. A 1-week washout is permitted for palliative radiation with a limited port ≤2 weeks of radiotherapy to non-central nervous system (CNS) disease.
3. The patient has received radiation therapy to the lung that is >30 Gray (Gy) within 6 months of the first dose of study medication.
4. The patient has received treatment with anticancer therapies including cytotoxic chemotherapy, monoclonal antibodies, and/or small molecule tyrosine kinase inhibitors within 14 days prior to study therapy administration, or 5 half-lives, whichever is shorter (42 days for prior nitrosourea or mitomycin-C; patients with advanced prostate cancer who are receiving luteinizing hormone releasing hormone (LHRH) agonists are permitted onto the study and should continue use of these agents during study treatment).
5. The patient has had an allogeneic tissue/solid organ transplant.
6. The patient has received a live or live-attenuated vaccine within 30 days prior to the first dose of study intervention. Administration of killed vaccines and RNA vaccines (e.g., COVID-19) are allowed.
7. The patient has received prior treatment with HMBD-002 or another investigational agent that targets VISTA.
8. If the participant had major surgery, the participant must have recovered adequately from the procedure and/or any complications from the surgery prior to starting study intervention.
9. The patient is currently participating in or has participated in a study of an investigational agent or has used an investigational device within 4 weeks prior to the first dose of study treatment.
10. The patient must have recovered from all AEs due to previous therapies to ≤Grade 1 or baseline. Participants with ≤Grade 2 alopecia and/or neuropathy may be eligible. Participants with endocrine-related AEs Grade ≤2 requiring treatment or hormone replacement may be eligible. Prior toxicities that resulted in laboratory abnormalities should have resolved to Grade ≤1 unless a higher-grade abnormality is allowed by the inclusion criteria. If medical therapy is required for the treatment of a laboratory abnormality, the dose and laboratory value(s) should be stable.
11. The patient has an active autoimmune disease that required systemic treatment in the past. Patients who have not required systemic treatment for at least two years may be enrolled if permission is provided after discussion with the Medical Monitor (replacement therapy, e.g., thyroxine, insulin, or physiologic corticosteroid replacement therapy for adrenal or pituitary insufficiency, is not considered a form of systemic treatment, and is allowed).
12. The patient has an uncontrolled endocrine disorder.
13. The patient has clinically significant cardiovascular disease (e.g., uncontrolled or any New York Heart Association Class 3 or 4 heart failure, uncontrolled angina, history of myocardial infarction, unstable angina or stroke within 6 months prior to study entry, uncontrolled hypertension or clinically significant arrhythmias not controlled by medication).
14. The patient has a history of (non-infectious) pneumonitis or interstitial pulmonary disease that required steroids or has current pneumonitis or interstitial pulmonary disease.
15. The patient has uncontrolled, clinically significant pulmonary disease (e.g., chronic obstructive pulmonary disease, pulmonary hypertension) that in the opinion of the Investigator would put the patient at significant risk for pulmonary complications during the study.
16. The patient has had a severe hypersensitivity reaction (≥Grade 3) to pembrolizumab and/or any of its excipients.
17. The patient has a diagnosis of immunodeficiency or is receiving chronic systemic steroid therapy (in dosing exceeding 10 mg daily of prednisone equivalent) or any other form of immunosuppressive therapy within 7 days prior the first dose of study drug. Inhaled or topical steroids are permitted in the absence of active autoimmune disease.
18. The patient has uncontrolled intercurrent illness including, but not limited to, uncontrolled infection requiring therapy, disseminated intravascular coagulation, psychiatric illness, drug abuse or a social situation that would limit compliance with study requirements.
19. Has a history or current evidence of any condition, therapy, or laboratory abnormality that might confound the results of the study, interfere with the participant's participation for the full duration of the study, or is not in the best interest of the participant to participate, in the opinion of the treating investigator.
20. The patient has known positive status for human immunodeficiency virus (HIV). No HIV testing is required unless mandated by local health authority.
21. Has a known history of Hepatitis B (defined as HBsAg reactive) or known active Hepatitis C virus (defined as HCV RNA detected) infection. No testing for Hepatitis B and Hepatitis C is required unless mandated by a local health authority.
22. The patient is oxygen dependent.
23. The patient has any medical condition which, in the opinion of the Investigator, places the patient at an unacceptably high risk for toxicities.
24. The patient must have a negative COVID test within 1 week of study treatment if patient is not fully vaccinated.
25. The patient has an additional active malignancy that is progressing or has required active treatment within the past 3 years. Patients with a past cancer history with substantial potential for recurrence must be discussed with the Medical Monitor before study entry. Patients with the following concomitant neoplastic diagnoses are eligible: non-melanoma skin cancer, carcinoma in situ (including transitional cell carcinoma, cervical intraepithelial neoplasia, breast cancer, and melanoma in situ), organ-confined prostate cancer with no evidence of progressive disease.
26. The patient has known active CNS metastases and/or carcinomatous meningitis. Participants with previously treated brain metastases may participate provided they are radiologically stable, i.e., without evidence of progression for at least 4 weeks by repeat imaging, clinically stable and without requirement of steroid treatment for at least 14 days prior to first dose of study treatment. For patients with a history of CNS involvement, the repeat imaging should be performed during study screening. However, CNS imaging is not required prior to study entry unless there is clinical suspicion of CNS involvement.
27. The patient is pregnant or breast feeding.

### 19.7.4 Safety Studies and Adverse Events

Safety will be assessed during the study by documentation of AEs, clinical laboratory tests, physical examination, vital sign (VS) measurements, electrocardiograms (ECGs), and Eastern Cooperative Oncology Group (ECOG) performance status (PS).

### Safety Assessments

Safety assessments include DLTs, AEs, serious AEs (SAEs), physical examinations, VS measurements, ECOG PS, clinical laboratory evaluations, and reasons for treatment discontinuation due to toxicity. The AE reporting period begins at the time the patient or authorized representative signs the ICF and continues through 90 days. All AEs that occur in treated patients during the AE reporting period specified in the protocol must be reported, whether the event is considered related to HMBD-002 or not. Any known untoward event that occurs beyond the AE reporting period that the Investigator assesses as related to HMBD-002 should also be reported as an AE.

### Efficacy Assessments

Efficacy assessments include disease response and progression, duration of response, and survival.

### Biological/Target/Correlative Studies

For patients in the dose escalation stage of the study, tumor tissue (unstained slides, or paraffin block) from the patient's most recent biopsy, must be available prior to treatment with study therapy. Slides from a fresh biopsy is preferred, however, if there are no archival tissues and if a biopsy is deemed by the investigator to not be in the patient's best interest (i.e., requires a significant risk procedure as defined as associated absolute risk of mortality or major morbidity in the patient's clinical setting and at the institution completing the procedure of 2% or higher), the Medical Monitor can waive this requirement.

For patients with TNBC, NSCLC, and other cancers, the investigator should obtain a biopsy in the screening period after giving informed consent for the study. Although documentation of a VISTA IHC+ tumor is not required to begin treatment, the biopsy procedure must not be a significant risk procedure, as defined as one for which the procedure associated absolute risk of mortality or major morbidity in the patient's clinical setting and at the institution completing the procedure is estimated to be 2% or higher. Otherwise, the patient will submit an archival tumor specimen. All biopsy procedure amenable patients, as previously described, will have a subsequent tumor biopsy performed in the week prior to the end of Cycle 2 (Days 16-21; ±3 days), or at PD, if PD is earlier.

Pre- and post-treatment biopsies, if they do not pose a significant procedure risk as defined above, will be used to evaluate VISTA and PD-L1 expression, as well as to study the effects of treatment with HMBD-002 on the expression of VISTA and PD-L1 receptors before treatment, during treatment, and post-treatment, and to correlate measurements with anti-tumor activity. In addition, the quantitative and qualitative effects of HMBD-002 on circulating immune cells and immune cells in the tumor microenvironment (TME) will be examined. An emphasis will be on the effects of treatment on VISTA IHC+ immune cells.

An AE is defined as any untoward medical occurrence in a clinical study patient administered a medicinal product, which does not necessarily have a causal relationship with this treatment. An AE can therefore be any unfavorable and unintended sign (including an abnormal laboratory finding), symptom, or disease temporally associated with the use of a medicinal (investigational) product, whether or not it is related to the medicinal (investigational) product. This includes an exacerbation of pre-existing conditions or events, intercurrent illnesses, drug interaction or the significant worsening of the indication under investigation that is not recorded elsewhere in the eCRF under specific efficacy assessments. Anticipated fluctuations of pre-existing conditions, including the disease (malignancy under study, that do not represent a clinically significant exacerbation or worsening need not be considered AEs.

Progression of the cancer under study is not considered an AE unless it results in hospitalization or death.

AE events will be deemed related to study medication unless clearly unrelated to study medication.

Severity of AEs will be graded according to NCI CTCAE version 5. If there is a change in severity of an AE, it must be recorded as a separate event. For AEs not listed included in the NCI CTCAE, then the severity can be determined as follows:

| | |
|---|---|
| Grade 1 (Mild) | Adverse event that disappears or is easily |
| | tolerated on continuation of study treatment. |
| Grade 2 (Moderate) | Adverse event sufficiently discomforting to |
| | cause interference with usual work activities. |
| Grade 3 (Severe) | Adverse event that is incapacitating, with |
| | inability to work or perform daily activities. |
| Grade 4 | Adverse event that is potentially |
| (Life-threatening) | life-threatening. |
| Grade 5 (Death) | Death related to the adverse event. |

### 19.8 PHARMACOKINETIC, PHARMACODYNAMIC AND BIOLOGIC ASSESSMENTS

Serial blood samples for PK and pharmacodynamic analyses will be collected from all patients.

Pharmacokinetics will be characterized for serum concentrations of HMBD-002 and will be analyzed using noncompartmental methods. Pharmacokinetic parameters to be calculated (if adequate data are available for estimation) will include, but not be limited to, Cₘₐₓ, AUC, t_{1/2}, CL, and V_{d}. Concentrations and PK parameters of HMBD-002 will be summarized for each dose-level cohort using descriptive statistics.

Dose Escalation Stage:
HMBD-002:
   An intensive schedule to determine serum concentrations of HMBD-002 will be performed in Cycles 1 and 2. In Cycle 1 blood will be sampled after treatment on Days 1 and 8 (pretreatment; at the end of the infusion, and at the following times post-infusion—1, 6, 24, 48, and 72 hours, Day 8 in monotherapy cohort only), as well as pretreatment and end of infusion on Day and 15 and in Cycle 2 at the following times: Day 1 (pretreatment; end of infusion, and at the following times post-infusion—1, 6, 24, 48, and 72 hours and Days 8 and 15 (pretreatment and end of infusion). If clinical data allows, the 72 hr PK timepoint may be eliminated. In essence, PK will be performed at two dose levels in all patients: Day 1 (33% or 50% dose) and Day 8 (100% dose). The same HMBD-002 PK sampling scheme will be used if the treatment schedule is modified to (Days 1, 8 every 3 weeks) or Day 1 every 3 weeks. In both cases, PK sampling will be performed on Days 8 and 15 (there will be no post-treatment sampling if drug treatment is not performed).
   Blood will also be sampled sparsely in subsequent cycles (pretreatment and end of infusion on Day 1 of Cycles 3-6, 8, 10, 15.

Dose Expansion Stage:
HMBD-002:
   Limited blood sampling will be performed to assess serum concentrations of HMBD-002 at the limited timepoints noted in Error! Reference source not found., Error! Reference source not found., Error! Reference source not found., Error! Reference source not found.
   Serum concentration data over time will be used to characterize the PK disposition of HMBD-002, to assess any change in the PK properties of HMBD-002 between initial administration and steady-state, and between cycles of treatment, and relate the PK characteristics of HMBD-002 to toxicity and anticancer activity.

Biomarkers will be assessed throughout the study.

Biospecimens (blood components, tumour material, etc.) will be collected to support analyses of cellular, circulating molecules and other biomarkers (e.g., protein, deoxyribonucleic acid [DNA], ribonucleic acid [RNA], metabolites). Changes in different blood and tumour biomarkers may provide evidence for biologic activity and may lead to insights that could guide use of HMBD-002.

Circulating serum cytokines may be measured, e.g. IL-2, IL-6, IL-8 and/or TNFα. Serum cytokines may be measured on Days 1, 8, 15 of Cycle 1 and Day 1 of Cycle 2.

Clinical evaluations will include measuring serum/blood levels of Creatinine, Blood urea nitrogen (BUN), Aspartate aminotransferase (AST or SGOT), Alanine aminotransferase (ALT or SGPT), Alkaline phosphatase (AP), Total bilirubin, Serum albumin, Serum total protein (TP), Sodium Bicarbonate, Potassium, Chloride, Glucose, Phosphate, Calcium, Triglycerides, C-Reactive Protein, Ferritin, Hemoglobin, Hematocrit, White blood cell (WBC) count (total and differential), Red blood cell (RBC) count, Platelet count, Mean corpuscular volume (MCV), Mean corpuscular hemoglobin (MCH), and Mean corpuscular hemoglobin concentration (MCHC).

Tumour tissue and blood sample may be characterized using genomic techniques (e.g., next generation sequencing and other technologies) to understand key molecular changes to VISTA-targeting agent. Both genome-wide and targeted messenger RNA (mRNA) expression profiling and sequencing in tumour tissue and in blood may be performed to define relevant gene signatures. Germline genetic analyses (e.g., Single Nucleotide Polymorphisms Analyses, Whole Exome Sequencing, Whole Genome Sequencing) within a clinical trial population may also be performed.

Blood and tumour samples from this study may undergo immune profiling and proteomic analyses. Tumour samples from this study may undergo proteomic analyses (e.g., VISTA, PD-L1 IHC) on immune and cancer cells. These tissue samples may also undergo digital pathology analysis and multiplex immunofluorescence. In addition, the presence and changes of tumour-infiltrating leukocytes, immune-related mRNA expression signatures, and VISTA and PD-L1 expression may also be assessed.

Tumour tissue and blood samples (included blood-derived proteins) may be subjected to proteomic analyses using a variety of platforms that could include but are not limited to immunoassays, flow cytometry, liquid chromatography/mass spectrometry. Assays such as enzyme-linked immunoassay measure such proteins in serum.

In addition, tumour and blood profiling outlined above, additional translational research may be conducted using components of biospecimens taken at Screening and following treatment. Proteins or cells from patients may be combined with laboratory reagents for additional ex vivo studies (e.g. cell proliferation, cytotoxicity etc.).

### Assessment of Anti-Drug Antibodies

All patients will be assessed for the development and quantitation of ADA to HMBD-002 in dose escalation and dose expansion stages. Blood sampling will be performed in pretreatment in Cycles1-6, and every 2 cycles thereafter and at the end of study (EOS).

### 19.9 ASSESSMENT OF ANTI-TUMOUR ACTIVITY

Tumour measurements will be taken throughout the study, e.g. as detailed below.

Disease progression and/or treatment efficacy is measured according to the Response Evaluation Criteria in Solid Tumours (RECIST) v1.1.

Overall response rate (ORR) is defined as the proportion of patients achieving a best response of complete response (CR) or partial response (PR) according to RECIST v.1.1. Duration of response (DoR) is defined as the time from the date measurement criteria are first met for PR or CR to the date measurement criteria are first met for PD. Duration of CR (DoCR) is defined as the time from the date when the measurement criteria are met for CR to the date measurement criteria are first met for PD. Progression-free survival (PFS) is defined as the time from the date of initiation of study therapy to the date measurement criteria are first met for PD or death from any cause, whichever occurs first. PFS6 is defined as the percentage of patients alive and progression-free at 6 months (26 weeks) after the initiation of study therapy. Overall survival (OS) is defined as the time from the date of initiation of study therapy to the date of death from any cause.

Testing for ORR and PFS6 will be by one-sample binomial tests with a 1-sided type I error rate of 15%. Distributions for PFS, DoR, DoCR, and OS will be estimated by Kaplan-Meier methodology.

New response evaluation criteria in solid tumours (RECIST criteria): Revised RECIST guideline (Version 1.1). E. A. Eisenhauer et al. (2009). European Journal of Cancer 45:228-247, which is hereby incorporated by reference in its entirety.

### 1. Measurability of Tumour at Baseline

### 1.1. Definitions

At baseline, tumour lesions/lymph nodes will be categorised measurable or non-measurable as follows:

### 1.1.1. Measurable

Tumour lesions: Must be accurately measured in at least one dimension (longest diameter in the plane of measurement is to be recorded) with a minimum size of:
10 mm by CT scan (CT scan slice thickness no greater than 5 mm; see Appendix II on imaging guidance).
10 mm calliper measurement by clinical exam (lesions which cannot be accurately measured with callipers should be recorded as non-measurable).
20 mm by chest X-ray

Malignant lymph nodes: To be considered pathologically enlarged and measurable, a lymph node must be 15 mm in the short axis when assessed by CT scan (CT scan slice thickness recommended to be no greater than 5 mm). At baseline and in follow-up, only the short axis will be measured and followed.

### 1.1.2. Non-Measurable

All other lesions, including small lesions (longest diameter <10 mm or pathological lymph nodes with 10 to <15 mm short axis) as well as truly non-measurable lesions. Lesions considered truly non-measurable include: leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

### 1.1.3. Special Considerations Regarding Lesion Measurability

Bone lesions, cystic lesions, and lesions previously treated with local therapy require particular comment:

### Bone Lesions:

Bone scan, PET scan or plain films are not considered adequate imaging techniques to measure bone lesions. However, these techniques can be used to confirm the presence or disappearance of bone lesions.
Lytic bone lesions or mixed lytic-blastic lesions, with identifiable soft tissue components, that can be evaluated by cross sectional imaging techniques such as CT or MRI can be considered as measurable lesions if the soft tissue component meets the definition of measurability described above.
Blastic bone lesions are non-measurable.

### Cystic Lesions:

Lesions that meet the criteria for radiographically defined simple cysts should not be considered as malignant lesions (neither measurable nor non-measurable) since they are, by definition, simple cysts.
‘Cystic lesions’ thought to represent cystic metastases can be considered as measurable lesions, if they meet the definition of measurability described above. However, if non-cystic lesions are present in the same patient, these are preferred for selection as target lesions.
   Lesions with Prior Local Treatment:
Tumour lesions situated in a previously irradiated area, or in an area subjected to other loco-regional therapy, are usually not considered measurable unless there has been demonstrated progression in the lesion. Trial protocols should detail the conditions under which such lesions would be considered measurable.

### 1.2. Specifications by Methods of Measurements

### 1.2.1. Measurement of Lesions

All measurements should be recorded in metric notation, using callipers if clinically assessed. All baseline evaluations should be performed as close as possible to the treatment start and never more than 4 weeks before the beginning of the treatment.

### 1.2.2. Method of Assessment

The same method of assessment and the same technique should be used to characterise each identified and reported lesion at baseline and during follow-up. Imaging based evaluation should always be done rather than clinical examination unless the lesion(s) being followed cannot be imaged but are assessable by clinical exam.

### Clinical Lesions:

Clinical lesions will only be considered measurable when they are superficial and ≥10 mm diameter as assessed using callipers (e.g. skin nodules). For the case of skin lesions, documentation by colour photography including a ruler to estimate the size of the lesion is suggested. As noted above, when lesions can be evaluated by both clinical exam and imaging, imaging evaluation should be undertaken since it is more objective and may also be reviewed at the end of the trial.

### Chest X-Ray:

Chest CT is preferred over chest X-ray, particularly when progression is an important endpoint, since CT is more sensitive than X-ray, particularly in identifying new lesions. However, lesions on chest X-ray may be considered measurable if they are clearly defined and surrounded by aerated lung.

### CT, MRI:

CT is the best currently available and reproducible method to measure lesions selected for response assessment. This guideline has defined measurability of lesions on CT scan based on the assumption that CT slice thickness is 5 mm or less. When CT scans have slice thickness greater than 5 mm, the minimum size for a measurable lesion should be twice the slice thickness. MRI is also acceptable in certain situations (e.g. for body scans). More details concerning the use of both CT and MRI for assessment of objective tumour response evaluation are provided in the publication from Eisenhauer et al.

### Ultrasound:

Ultrasound is not useful in assessment of lesion size and should not be used as a method of measurement. Ultrasound examinations cannot be reproduced in their entirety for independent review at a later date and, because they are operator dependent, it cannot be guaranteed that the same technique and measurements will be taken from one assessment to the next (described in greater detail in Appendix II). If new lesions are identified by ultrasound in the course of the trial, confirmation by CT or MRI is advised. If there is concern about radiation exposure at CT, MRI may be used instead of CT in selected instances.

### Endoscopy, Laparoscopy:

The utilisation of these techniques for objective tumour evaluation is not advised. However, they can be useful to confirm complete pathological response when biopsies are obtained or to determine relapse in trials where recurrence following complete response or surgical resection is an endpoint.

### Tumour Markers:

Tumour markers alone cannot be used to assess objective tumour response. If markers are initially above the upper normal limit, however, they must normalise for a patient to be considered in complete response.

### Cytology, Histology:

These techniques can be used to differentiate between PR and CR in rare cases if required by protocol (for example, residual lesions in tumour types such as germ cell tumours, where known residual benign tumours can remain). When effusions are known to be a potential adverse effect of treatment (e.g. with certain taxane compounds or angiogenesis inhibitors), the cytological confirmation of the neoplastic origin of any effusion that appears or worsens during treatment can be considered if the measurable tumour has met criteria for response or stable disease in order to differentiate between response (or stable disease) and progressive disease.

### 2. Tumour Response Evaluation

### 2.1 Assessment of Overall Tumour Burden and Measurable Disease

To assess objective response or future progression, it is necessary to estimate the overall tumour burden at baseline and use this as a comparator for subsequent measurements. Measurable disease is defined by the presence of at least one measurable lesion (as detailed above).

### 2.2. Baseline Documentation of ‘Target’ and ‘Non-Target’ Lesions

When more than one measurable lesion is present at baseline, all lesions up to a maximum of 5 lesions total (and a maximum of 2 lesions per organ) representative of all involved organs should be identified as target lesions and should be recorded and measured at baseline.

This means in instances where patients have only 1 or 2 organ sites involved a maximum of 2 (1 site) and 4 lesions (2 sites), respectively, will be recorded. Other lesions in that organ will be recorded as non-measurable lesions (even if their size is greater than 10 mm by CT scan).

Target lesions should be selected on the basis of their size (lesions with the longest diameter), be representative of all involved organs, but in addition, should be those that lend themselves to reproducible repeated measurements. It may be the case that, on occasion, the largest lesion does not lend itself to reproducible measurement in which circumstance the next largest lesion which can be measured reproducibly should be selected.

Lymph nodes merit special mention since they are normal anatomical structures which may be visible by imaging even if not involved by tumor. Pathological nodes which are defined as measurable and may be identified as target lesions must meet the criterion of a short axis of 15 mm by CT scan. Only the short axis of these nodes will contribute to the baseline sum. The short axis of the node is the diameter normally used by radiologists to judge if a node is involved by solid tumor. Nodal size is normally reported as 2 dimensions in the plane in which the image is obtained (for CT scan this is almost always the axial plane; for MRI the plane of acquisition may be axial, sagittal or coronal). The smaller of these measures is the short axis. For example, an abdominal node which is reported as being 20 mm×30 mm has a short axis of 20 mm and qualifies as a malignant, measurable node. In this example, 20 mm should be recorded as the node measurement. All other pathological nodes (those with short axis □10 mm but <15 mm) should be considered non-target lesions. Nodes that have a short axis <10 mm are considered non-pathological and should not be recorded or followed.

A sum of the diameters (longest for non-nodal lesions, short axis for nodal lesions) for all target lesions will be calculated and reported as the baseline sum diameters. If lymph nodes are to be included in the sum, then as noted above, only the short axis is added into the sum. The baseline sum diameters will be used as reference to further characterize any objective tumor regression in the measurable dimension of the disease.

All other lesions (or sites of disease) including pathological lymph nodes should be identified as non-target lesions and should also be recorded at baseline. Measurements are not required and these lesions should be followed as “present”, “absent”, or in rare cases “unequivocal progression”. In addition, it is possible to record multiple non-target lesions involving the same organ as a single item on the case report form (e.g., “multiple enlarged pelvic lymph nodes” or “multiple liver metastases”).

### 2.3. Response Criteria

### 2.3.1. Evaluation of Target Lesions

Complete Response (CR): Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Stable Disease (SD): Neither sufficient shrinkage to qualify for PR nor sufficient increase to qualify for PD, taking as reference the smallest sum diameters while on study.

### 2.3.2. Special Notes on the Assessment of Target Lesions

Lymph nodes: Lymph nodes identified as target lesions should always have the actual short axis measurement recorded (measured in the same anatomical plane as the baseline examination), even if the nodes regress to below 10 mm on study. This means that when lymph nodes are included as target lesions, the “sum” of lesions may not be zero even if CR criteria are met, since a normal lymph node is defined as having a short axis of <10 mm. Electronic case report forms (eCRFs) or other data collection methods may therefore be designed to have target nodal lesions recorded in a separate section where, in order to qualify for CR, each node must achieve a short axis <10 mm. For PR, SD, and PD, the actual short axis measurement of the nodes is to be included in the sum of target lesions.

Target lesions that become “too small to measure”: While on study, all lesions (nodal and non-nodal) recorded at baseline should have their actual measurements recorded at each subsequent evaluation, even when very small (e.g., 2 mm). However, sometimes lesions or lymph nodes which are recorded as target lesions at baseline become so faint on CT scan that the radiologist may not feel comfortable assigning an exact measure and may report them as being “too small to measure”. When this occurs, it is important that a value be recorded on the eCRF:
If it is the opinion of the radiologist that the lesion has likely disappeared, the measurement should be recorded as 0 mm. If the lesion is believed to be present and is faintly seen but too small to measure, a default value of 5 mm should be assigned and “below measurable limit” (BML) should be ticked (Note: It is less likely that this rule will be used for lymph nodes since they usually have a definable size when normal and are frequently surrounded by fat such as in the retroperitoneum; however, if a lymph node is believed to be present and is faintly seen but too small to measure, a default value of 5 mm should be assigned in this circumstance as well and BML should also be ticked).

This default value is derived from the 5 mm CT slice thickness (but should not be changed with varying CT slice thickness). The measurement of these lesions is potentially non-reproducible, therefore providing this default value will prevent false responses or progressions based upon measurement error.

Lesions that split or coalesce on treatment: When non-nodal lesions “fragment”, the longest diameters of the fragmented portions should be added together to calculate the target lesion sum. Similarly, as lesions coalesce, a plane between them may be maintained that would aid in obtaining maximal diameter measurements of each individual lesion. If the lesions have truly coalesced such that they are no longer separable, the vector of the longest diameter in this instance should be the maximal longest diameter for the “coalesced lesion”.

### 2.3.3. Evaluation of Non-Target Lesions

While some non-target lesions may actually be measurable, they need not be measured and instead should be assessed only qualitatively at the time points specified in the protocol.

Complete Response: Disappearance of all non-target lesions and normalization of tumor marker level. All lymph nodes must be non-pathological in size (<10 mm short axis).

Non-CR/Non-PD: Persistence of one or more non-target lesion(s) and/or maintenance of tumor marker level above the normal limits.

Progressive Disease: Unequivocal progression of existing non-target lesions. (Note: the appearance of one or more new lesions is also considered progression).

### 2.3.4. Special Notes on Assessment of Progression of Non-Target Disease

The concept of progression of non-target disease requires additional explanation as follows:

When the patient also has measurable disease:

In this setting, to achieve ‘unequivocal progression’ on the basis of the non-target disease, there must be an overall level of substantial worsening in non-target disease such that, even in presence of SD or PR in target disease, the overall tumour burden has increased sufficiently to merit discontinuation of therapy. A modest ‘increase’ in the size of one or more non-target lesions is usually not sufficient to quality for unequivocal progression status. The designation of overall progression solely on the basis of change in non-target disease in the face of SD or PR of target disease will therefore be extremely rare.

When the patient has only non-measurable disease:

This circumstance arises in some phase III trials when it is not a criterion of trial entry to have measurable disease. The same general concepts apply here as noted above, however, in this instance there is no measurable disease assessment to factor into the interpretation of an increase in non-measurable disease burden. Because worsening in non-target disease cannot be easily quantified (by definition: if all lesions are truly non-measurable) a useful test that can be applied when assessing patients for unequivocal progression is to consider if the increase in overall disease burden based on the change in non-measurable disease is comparable in magnitude to the increase that would be required to declare PD for measurable disease: i.e. an increase in tumour burden representing an additional 73% increase in ‘volume’ (which is equivalent to a 20% increase diameter in a measurable lesion). Examples include an increase in a pleural effusion from ‘trace’ to ‘large’, an increase in lymphangitic disease from localised to widespread, or may be described in protocols as ‘sufficient to require a change in therapy’. If ‘unequivocal progression’ is seen, the patient should be considered to have had overall PD at that point. While it would be ideal to have objective criteria to apply to non-measurable disease, the very nature of that disease makes it impossible to do so; therefore the increase must be substantial.

### 2.3.5. New Lesions

The appearance of new malignant lesions denotes disease progression; therefore, some comments on detection of new lesions are important. There are no specific criteria for the identification of new radiographic lesions; however, the finding of a new lesion should be unequivocal: i.e. not attributable to differences in scanning technique, change in imaging modality or findings thought to represent something other than tumour (for example, some ‘new’ bone lesions may be simply healing or flare of pre-existing lesions). This is particularly important when the patient's baseline lesions show partial or complete response. For example, necrosis of a liver lesion may be reported on a CT scan report as a ‘new’ cystic lesion, which it is not.

A lesion identified on a follow-up trial in an anatomical location that was not scanned at baseline is considered a new lesion and will indicate disease progression. An example of this is the patient who has visceral disease at baseline and while on trial has a CT or MRI brain ordered which reveals metastases. The patient's brain metastases are considered to be evidence of PD even if he/she did not have brain imaging at baseline.

If a new lesion is equivocal, for example because of its small size, continued therapy and follow-up evaluation will clarify if it represents truly new disease. If repeat scans confirm there is definitely a new lesion, then progression should be declared using the date of the initial scan.

(18) F-Fluorodeoxyglucose Positron Emission Tomography (FDG-PET): For the purposes of this study, progressive disease should not be made solely on FDG-PET findings. All FDG-PET findings suggestive of progressive disease should be confirmed by dedicated anatomic imaging (CT or MRI). The following modifications to RECIST v1.1. will be applied to this study:
Negative FDG-PET at baseline, with a positive FDG-PET at follow-up is a sign of PD based on a new lesion. * Confirmation of the new lesion by CT or MRI scan is required per protocol.
No FDG-PET at baseline and a positive FDG-PET at follow-up:
   If the positive FDG-PET at follow-up corresponds to a new sign of disease confirmed by CT, this is PD
   If the positive FDG-PET at follow-up is not confirmed as a new site of disease on CT, additional follow-up CT scans are needed to determine if there is truly progression occurring at that site (if so, the date of PD will be the date of the initial abnormal *CT scan).
   If the positive FDG-PET at follow-up corresponds to a pre-existing site of disease on CT that is not progressing on the basis of the anatomic images, this is not PD.
      *reflects study-specific modification to RECIST v.1.1

### 2.3.6. Evaluation of Best Overall Response

The best overall response is the best response recorded from the start of the study treatment until the end of treatment considering any requirement for confirmation. On occasion a response may not be documented until after the end of therapy so protocols should be clear if post-treatment assessments are to be considered in determination of best overall response. Protocols must specify how any new therapy introduced before progression will affect best response designation. The patient's best overall response assignment will depend on the findings of both target and non-target disease and will also take into consideration the appearance of new lesions. Furthermore, depending on the nature of the study and the protocol requirements, it may also require confirmatory measurement. Specifically, in nonrandomized trials where response is the primary endpoint, confirmation of PR or CR is needed to deem either one the ‘best overall response’. This is described further below.

### 19.10 REFERENCES FOR EXAMPLE 19

All references are hereby incorporated herein in their entirety.
1. Wang L, Rubinstein R, Lines J L, Wasiuk A, Ahonen C, Guo Y, Lu L F, Gondek D, Wang Y, Fava R A, Fiser A, Almo S, Noelle R J: VISTA, a novel mouse Ig superfamily ligand that negatively regulates T cell responses. J Exp Med 208:577-92, 2011.
2. Flies D B, Wang S, Xu H, Chen L: Cutting edge: A monoclonal antibody specific for the programmed death-1 homolog prevents graft-versus-host disease in mouse models. J Immunol 187:1537-41, 2011.
3. Mehta N, Maddineni S, Mathews I I, Sperberg A P R, Huang P S, Cochran J R: Structure and Functional Binding Epitope of V-domain Ig Suppressor of T Cell Activation. Cell Rep 28:2509-2516 e5, 2019.
4. Johnston R J, Su L J, Pinckney J, Critton D, Boyer E, Krishnakumar A, Corbett M, Rankin A L, Dibella R, Campbell L, Martin G H, Lemar H, Cayton T, Huang R Y, Deng X, Nayeem A, Chen H, Ergel B, Rizzo J M, Yamniuk A P, Dutta S, Ngo J, Shorts A O, Ramakrishnan R, Kozhich A, Holloway J, Fang H, Wang Y K, Yang Z, Thiam K, Rakestraw G, Rajpal A, Sheppard P, Quigley M, Bahjat K S, Korman A J: VISTA is an acidic pH-selective ligand for PSGL-1. Nature 574:565-570, 2019.
5. Li N, Xu W, Yuan Y, Ayithan N, Imai Y, Wu X, Miller H, Olson M, Feng Y, Huang Y H, Jo Turk M, Hwang S T, Malarkannan S, Wang L: Immune-checkpoint protein VISTA critically regulates the IL-23/IL-17 inflammatory axis. Sci Rep 7:1485, 2017.
6. Borggrewe M, Grit C, Den Dunnen W F A, Burm S M, Bajramovic J J, Noelle R J, Eggen B J L, Laman J D: VISTA expression by microglia decreases during inflammation and is differentially regulated in CNS diseases. Glia 66:2645-2658, 2018.
7. Han X, Vesely M D, Yang W, Sanmamed M F, Badri T, Alawa J, Lopez-Giraldez F, Gaule P, Lee S W, Zhang J P, Nie X, Nassar A, Boto A, Flies D B, Zheng L, Kim T K, Moeckel G W, McNiff J M, Chen L: PD-1H (VISTA)-mediated suppression of autoimmunity in systemic and cutaneous lupus erythematosus. Sci Transl Med 11, 2019.
8. Sergent P A, Plummer S F, Pettus J, Mabaera R, DeLong J K, Pechenick D A, Burns C M, Noelle R J, Ceeraz S: Blocking the VISTA pathway enhances disease progression in (NZB×NZW) F1 female mice. Lupus 27:210-216, 2018.
9. Yoon K W, Byun S, Kwon E, Hwang S Y, Chu K, Hiraki M, Jo S H, Weins A, Hakroush S, Cebulla A, Sykes D B, Greka A, Mundel P, Fisher D E, Mandinova A, Lee S W: Control of signaling-mediated clearance of apoptotic cells by the tumor suppressor p53. Science 349:1261669, 2015.
10. Liu J, Yuan Y, Chen W, Putra J, Suriawinata A A, Schenk A D, Miller H E, Guleria I, Barth R J, Huang Y H, Wang L: Immune-checkpoint proteins VISTA and PD-1 nonredundantly regulate murine T-cell responses. Proc Natl Acad Sci USA 112:6682-7, 2015.
11. Keir M E, Freeman G J, Sharpe A H: PD-1 regulates self-reactive CD8+ T cell responses to antigen in lymph nodes and tissues. J Immunol 179:5064-70, 2007.
12. Wang G, Tai R, Wu Y, Yang S, Wang J, Yu X, Lei L, Shan Z, Li N: The expression and immunoregulation of immune checkpoint molecule VISTA in autoimmune diseases and cancers. Cytokine Growth Factor Rev, 2020.
13. El Tanbouly M A, Zhao Y, Nowak E, Li J, Schaafsma E, Le Mercier I, Ceeraz S, Lines J L, Peng C, Carriere C, Huang X, Day M, Koehn B, Lee S W, Silva Morales M, Hogquist K A, Jameson S C, Mueller D, Rothstein J, Blazar B R, Cheng C, Noelle R J: VISTA is a checkpoint regulator for naive T cell quiescence and peripheral tolerance. Science 367, 2020.
14. Le Mercier I, Chen W, Lines J L, Day M, Li J, Sergent P, Noelle R J, Wang L: VISTA Regulates the Development of Protective Antitumor Immunity. Cancer Res 74:1933-44, 2014.
15. Borggrewe M, Kooistra S M, Noelle R J, Eggen B J L, Laman J D: Exploring the VISTA of microglia: immune checkpoints in CNS inflammation. J Mol Med (Berl). 2020 Aug. 27. doi: 10.1007/s00109-020-01968-x. Online ahead of print.
16. Li Wang 1, Rotem Rubinstein, Janet L Lines, Anna Wasiuk, Cory Ahonen, Yanxia Guo, Li-Fan Lu, David Gondek, Yan Wang, Roy A Fava, Andras Fiser, Steve Almo, Randolph J Noelle. VISTA, a novel mouse Ig superfamily ligand that negatively regulates T cell responses. J Exp Med 208(3): 577-92, 2011.
17. Gao J, Ward J F, Pettaway C A, Shi L Z, Subudhi S K, Vence L M, Zhao H, Chen J, Chen H, Efstathiou E, Troncoso P, Allison J P, Logothetis C J, Wistuba, I I, Sepulveda M A, Sun J, Wargo J, Blando J, Sharma P: VISTA is an inhibitory immune checkpoint that is increased after ipilimumab therapy in patients with prostate cancer. Nat Med 23:551-555, 2017.
18. Kakavand H, Jackett L A, Menzies A M, Gide T N, Carlino M S, Saw RPM, Thompson J F, Wilmott J S, Long G V, Scolyer R A: Negative immune checkpoint regulation by VISTA: a mechanism of acquired resistance to anti-PD-1 therapy in metastatic melanoma patients. Mod Pathol 30:1666-1676, 2017.
19. Xu W, Hiéu T, Malarkannan S, Wang L. The structure, expression, and multifaceted role of immune-checkpoint protein VISTA as a critical regulator of anti-tumor immunity, autoimmunity, and inflammation. Cell Mol Immunol. 15(5): 438-446, 2018.
20. Wang J, Wu G, Manick B, Hernandez V, Renelt M, Erickson C, Guan J, Singh R, Rollins S, Solorz A, Bi M, Li J, Grabowski D, Dirkx J, Tracy C, Stuart T, Ellinghuysen C, Desmond D, Foster C, Kalabokis V: VSIG-3 as a ligand of VISTA inhibits human T-cell function. Immunology 156:74-85, 2019.
21. Mulati K, Hamanishi J, Matsumura N, Chamoto K, Mise N, Abiko K, Baba T, Yamaguchi K, Horikawa N, Murakami R, Taki M, Budiman K, Zeng X, Hosoe Y, Azuma M, Konishi I, Mandai M: VISTA expressed in tumour cells regulates T cell function. Br J Cancer 120:115-127, 2019.
22. Kondo Y, Ohno T, Nishii N, Harada K, Yagita H, Azuma M: Differential contribution of three immune checkpoint (VISTA, CTLA-4, PD-1) pathways to antitumor responses against squamous cell carcinoma. Oral Oncol 57:54-60, 2016.
23. Blando J, Sharma A, Higa M G, Zhao H, Vence L, Yadav S S, Kim J, Sepulveda A M, Sharp M, Maitra A, Wargo J, Tetzlaff M, Broaddus R, Katz MHG, Varadhachary G R, Overman M, Wang H, Yee C, Bernatchez C, Iacobuzio-Donahue C, Basu S, Allison J P, Sharma P: Comparison of immune infiltrates in melanoma and pancreatic cancer highlights VISTA as a potential target in pancreatic cancer. Proc Natl Acad Sci USA 116:1692-1697, 2019.
24. Muller S, Victoria Lai W, Adusumilli P S, Desmeules P, Frosina D, Jungbluth A, Ni A, Eguchi T, Travis W D, Ladanyi M, Zauderer M G, Sauter J L: V-domain Ig-containing suppressor of T-cell activation (VISTA), a potentially targetable immune checkpoint molecule, is highly expressed in epithelioid malignant pleural mesothelioma. Mod Pathol 33:303-311, 2020.
25. Chung Y S, Kim M, Cha Y J, Kim K A, Shim H S: Expression of V-set immunoregulatory receptor in malignant mesothelioma. Mod Pathol 33:263-270, 2020.
26. Hmeljak J, Sanchez-Vega F, Hoadley K A, Shih J, Stewart C, Heiman D, Tarpey P, Danilova L, Drill E, Gibb E A, Bowlby R, Kanchi R, Osmanbeyoglu H U, Sekido Y, Takeshita J, Newton Y, Graim K, Gupta M, Gay C M, Diao L, Gibbs D L, Thorsson V, Iype L, Kantheti H, Severson D T, Ravegnini G, Desmeules P, Jungbluth A A, Travis W D, Dacic S, Chirieac L R, Galateau-Salle F, Fujimoto J, Husain A N, Silveira H C, Rusch V W, Rintoul R C, Pass H, Kindler H, Zauderer M G, Kwiatkowski D J, Bueno R, Tsao A S, Creaney J, Lichtenberg T, Leraas K, Bowen J, Network T R, Felau I, Zenklusen J C, Akbani R, Cherniack A D, Byers L A, Noble M S, Fletcher J A, Robertson A G, Shen R, Aburatani H, Robinson B W, Campbell P, Ladanyi M: Integrative Molecular Characterization of Malignant Pleural Mesothelioma. Cancer Discov 8:1548-1565, 2018.
27. Villarroel-Espindola, F., Yu, X., Datar, I., Mani, N., Sanmamed, M. F., Velcheti, V., . . . . Schalper, K. A. (2017). Spatially resolved and quantitative analysis of VISTA/PD-1H as a novel immunotherapy target in human non-small cell lung cancer. Clinical Cancer Research, 24(7), clincanres.2542.2017
28. Brcic L, Stanzer S, Krenbek D, Gruber-Moesenbacher U, Absenger G, Quehenberger F, Valipour A, Lindenmann J, Stoeger H, Al Effah M, Fediuk M, Balic M, Popper H H: Immune cell landscape in therapy-naive squamous cell and adenocarcinomas of the lung. Virchows Arch 472:589-598, 2018.
29. Xiong D, Pan J, Yin Y, Jiang H, Szabo E, Lubet R A, Wang Y, You M: Novel mutational landscapes and expression signatures of lung squamous cell carcinoma. Oncotarget 9:7424-7441, 2018.
30. Manzoni M, Bolognesi M M, Antoranz A, Mancari R, Carinelli S, Faretta M, Bosisio F M, Cattoretti G: The Adaptive and Innate Immune Cell Landscape of Uterine Leiomyosarcomas. Sci Rep 10:702, 2020.
31. Bang Y J, Sosman J, Daud A, Meric-Bernstam F, Garcia-Corbacho J, Patel M, Lee J, Kim K, Brody J, Rha S Y, Martin M G, Aix S P, Hamilton E, Ramchandren R, Ahn M, Spicer J, Pacey S, Falchook G, Wang H, Xu G, Adams L, Ma AWS, Gharavi R, Eves P T, Tuck D, Powderly J: Phase 1 study of CA-170, a first-in-class, orally available, small molecule immune checkpoint inhibitor (ICI) dually targeting VISTA and PDL1, in patients with advanced solid tumors or lymphomas, SITC, 2018
32. Radhakrishnan V, Banavali S, Gupta S, Kumar A, Deshmukh C, Nag S, Beniwal S, Gopichand M, Naik R, Lakshmaiah K, Mandavia D, Ramachandra M, Prabhash K: Excellent CBR and prolonged PFS in non-squamous NSCLC with oral CA-170, a dual inhibitor of VISTA and P D-L1, ESMO, 2019
33. Zauderer M G, Brody J, Marron T, Pacey S, Martell R E, Wang H, Spicer J: First-in-Class Small Molecule CA-170 Targeting VISTA: A Report on Efficacy Outcomes from a Cohort of 12 Malignant Pleural Mesothelioma (MPM) Patients in Study CA-170-101, SITC 2019. National Harbor, M D, 2019.
34. Musielak B, Kocik J, Skalniak L, Magiera-Mularz K, Sala D, Czub M, Stec M, Siedlar M, Holak T A, Plewka J: CA-170-A Potent Small-Molecule P D-L1 Inhibitor or Not? Molecules 24, 2019.
35. Blevins D J, Hanley R, Bolduc T, et al. In Vitro Assessment of Putative PD-1/P D-L1 Inhibitors: Suggestions of an Alternative Mode of Action. ACS Medicinal Chemistry Letters; 10 (8), 1187-1192, 2019. DOI: 10.1021/acsmedchemlett.9b00221
35. Crawley S C, Hsu J Y, Tian H, Compositions and methods for targeting a pathway. Patent Cooperation Treaty (PCT) number PCT/US2015/031539. Information available from: https://patentscope.wipo.int/search/en/detail.jsf?docld=WO2015187359 (2015).
36. Li K, Tian H. Development of small-molecule immune checkpoint inhibitors of PD-1/PD-L1 as a new therapeutic strategy for tumour immunotherapy. J Drug Target. 2019 March; 27(3): 244-256. Review.
37. Mahoney K M, Freeman G J. Acidity changes immunology: a new VISTA pathway. Nat Immunol. 2020 January; 21(1): 13-16.
38. KEYTRUDA™ (pembrolizumab), Merck Sharp & Dohme Corp. US Prescribing Information, 05/2021. https://www.merck.com/product/usa/pi_circulars/k/keytruda/keytruda_pi.pdf
39. ClinicalTrials.gov [Internet]. Bethesda (MD): National Library of Medicine (US). Identifier NCT02671955, A study of safety, pharmacokinetics, pharmacodynamics of JNJ-61610588 in participants with advanced cancer; 2016 Feb. 2. Available from: https://clinicaltrials.gov/ct2/show/NCT02671955
40. ClinicalTrials.gov [Internet]. Bethesda (MD): National Library of Medicine (US). Identifier NCT04475523, Phase 1 study of CI-8993 anti-VISTA antibody in patients with advanced solid tumor malignancies; 2020 Jul. 17. Available from: https://clinicaltrials.gov/ct2/show/NCT02671955
41. ClinicalTrials.gov [Internet]. Bethesda (MD): National Library of Medicine (US). Identifier NCT04564417, First-in-human (FIH) study of w0180 as single agent and in combination with pembrolizumab in adults with locally advanced or metastatic solid tumors; 2020 Sep. 25. Available from: https://clinicaltrials.gov/ct2/show/NCT02671955
42. ClinicalTrials.gov [Internet]. Bethesda (MD): National Library of Medicine (US). Identifier NCT02812875, A study of ca-170 (oral pd-11, pd-12 and vista checkpoint antagonist) in patients with advanced tumors and lymphomas; 2016 Jun. 24. Available from: https://clinicaltrials.gov/ct2/show/NCT02671955
43. Disis, Mary L. Immune Regulation of Cancer. Journal of Clinical Oncology. 2010; 28(29): 4531-4538
44. Dudley, Mark E., Wunderlich, John E., Yang, James C., Sherry, Richard M., Topalian, Suzzane L., Restifo, Nicholas P., Royal, Richard E., Kammula, Udai, White, Don E., Mavroukakis, Sharon A., et al. Adoptive Cell Transfer Therapy Following Non-Myeloablative but Lymphodepleting Chemotherapy for the Treatment of Patients With Refractory Metastatic Melanoma. Journal of Clinical Oncology. 2005; 0732-183
45. Hunder N N, Wallen H, Cao J, Hendricks D W, Reilly J Z, Rodmyre R, et al. Treatment of metastatic melanoma with autologous CD4+ T cells against NY-ESO-1. N Engl J Med 2008; 358(25): 2698-703
46. Greenwald, Rebecca J., Freeman, Gordon J., and Sharpe, Arlene H. The B7 Family Revisited. Annual Reviews. 2005; 23:515-48
47. Okazaki T, Maeda A, Nishimura H, Kurosaki T, Honjo T. PD-1 immunoreceptor inhibits B cell receptor-mediated signaling by recruiting src homology 2-domain-containing tyrosine phosphatase 2 to phosphotyrosine. Proc Natl Acad Sci USA 2001; 98(24): 13866-71.
48. Zhang, P D., Xuewu, Schwartz, Jean-Claude D., Guo, Xiaoling, Bhatia, Sumeena, Cao, Erhu, Chen, Lieping, Zhang, Zhong-Yin, Edidin, Michael A., Nathenson, Stanley G. Almo, Steven C. Structural and Functional Analysis of the Costimulatory Receptor Programmed Death-1. Immunity. 2004; 337-347
49. Chemnitz, Jens M., Parry, Richard V., Nichols, Kim E., June, Carl H., Riley, James L., SHP-1 and SHP-2 Associate with Immunoreceptor Tyrosine-Based Switch Motif of Programmed Death 1 upon Primary Human T Cell Stimulation, but Only Receptor Ligation Prevents T Cell Activation. The Journal of Immunology. 2004, 173:945-954
50. Shepard, Kelly-Ann., Fitz, Lori J., Lee, Julie M., Benander, Christina, George, Judith A., Wooter, Joe, Qiu, Yongchang, Jussif, Jason M., Carter, Laura L., Wood, Clive R., Chaudhary, Divya. PD-1 inhibits T-cell receptor induced phosphorylation of the ZAP70/CD3 signalosome and downstream signaling to PKC. FEBS Letters. 2004; 0014-5793
51. Riley, James L., PD-1 signaling in primary T cells. Immunological Reviews. 2009; 0105-2896
52. Parry, Richard V., Chemnitz, Jens M., Frauwirth, Kenneth A., Lanfranco, Anthony R., Braunstein, Inbal., Kobayashi, Sumire V., Linsley, Peter S., Thompson, 4 Craig B. and Riley, James L. CTLA-4 and PD-1 Receptors Inhibit T-Cell Activation by Distinct Mechanisms. MOLECULAR AND CELLULAR BIOLOGY, 2005; 0270-7306Pilon-Thomas, S., et al., Blockade of programmed death ligand 1 enhances the therapeutic efficacy of combination immunotherapy against melanoma. J Immunol, 2010. 184(7): p. 3442-9.
53. Francisco, Loise M., Sage, Peter T., and Sharpe, Arlene H. The PD-1 pathway in tolerance and autoimmunity. Immunological Reviews. 2010; 0105-2896
54. Hirano, F., et al., Blockade of B7-H1 and PD-1 by monoclonal antibodies potentiates cancer therapeutic immunity. Cancer Res, 2005. 65(3): p. 1089-96.
55. Blank, C., et al., PD-L1/B7H-1 inhibits the effector phase of tumor rejection by T cell receptor (TCR) transgenic CD8+ T cells. Cancer Res, 2004. 64(3): p. 1140-5.
56. Weber, J., Immune checkpoint proteins: a new therapeutic paradigm for cancer--preclinical background: CTLA-4 and PD-1 blockade. Semin Oncol, 2010. 37(5): p. 430-9.
57. Strome, S. E., et al., B7-H1 blockade augments adoptive T-cell immunotherapy for squamous cell carcinoma. Cancer Res, 2003. 63(19): p. 6501-5.
58. Spranger, S., et al., Mechanism of tumor rejection with doublets of CTLA-4, PD-1/PD-L1, or IDO blockade involves restored IL-2 production and proliferation of CD8 (+) T cells directly within the tumor microenvironment. J Immunother Cancer, 2014. 2: p. 3.
59. Curran, M. A., et al., PD-1 and CTLA-4 combination blockade expands infiltrating T cells and reduces regulatory T and myeloid cells within B16 melanoma tumors. Proc Natl Acad Sci USA, 2010. 107(9): p. 4275-80.
60. Pilon-Thomas, S., et al., Blockade of programmed death ligand 1 enhances the therapeutic efficacy of combination immunotherapy against melanoma. J Immunol, 2010. 184(7): p. 3442-9.
61. Nomi, T., et al., Clinical significance and therapeutic potential of the programmed death-1 ligand/programmed death-1 pathway in human pancreatic cancer. Clin Cancer Res, 2007. 13(7): p. 2151-7.

1. An antigen-binding molecule, optionally isolated, which is capable of binding to VISTA and inhibiting VISTA-mediated signalling, independently of Fc-mediated function.

2. The antigen-binding molecule according to claim 1, wherein the antigen-binding molecule comprises:
(i) a heavy chain variable (VH) region incorporating the following CDRs:
HC-CDR 1 having the amino acid sequence of SEQ ID NO:290
HC-CDR2 having the amino acid sequence of SEQ ID NO:291
HC-CDR3 having the amino acid sequence of SEQ ID NO:278; and
(ii) a light chain variable (VL) region incorporating the following CDRs:
LC-CDR1 having the amino acid sequence of SEQ ID NO:41
LC-CDR2 having the amino acid sequence of SEQ ID NO:309
LC-CDR3 having the amino acid sequence of SEQ ID NO:43.

3. The antigen-binding molecule according to claim 1, wherein the antigen-binding molecule comprises:
(i) a heavy chain variable (VH) region incorporating the following CDRs:
HC-CDR1 having the amino acid sequence of SEQ ID NO:290
HC-CDR2 having the amino acid sequence of SEQ ID NO:291
HC-CDR3 having the amino acid sequence of SEQ ID NO:278; and
(ii) a light chain variable (VL) region incorporating the following CDRs:
LC-CDR1 having the amino acid sequence of SEQ ID NO:41
LC-CDR2 having the amino acid sequence of SEQ ID NO:295
LC-CDR3 having the amino acid sequence of SEQ ID NO:43.

4. The antigen-binding molecule according to claim 1, wherein the antigen-binding molecule comprises:
(i) a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:310; or
(ii) a VH region comprising an amino acid sequence having at least 70% sequence identity to the amino acid sequence of SEQ ID NO:289; and
a VL region comprising an amino acid sequence having at least 70% sequence identity to the amino acid sequence of SEQ ID NO:297.

5. (canceled)

6. The antigen-binding molecule according to claim 1, wherein the antigen-binding molecule comprises:
a VH region incorporating the following framework regions (FRs):
HC-FR1 having the amino acid sequence of SEQ ID NO:63
HC-FR2 having the amino acid sequence of SEQ ID NO:292
HC-FR3 having the amino acid sequence of SEQ ID NO:293
HC-FR4 having the amino acid sequence of SEQ ID NO:281.

7. The composition according to claim 1, wherein the antigen-binding molecule comprises:
a VL region incorporating the following framework regions (FRs):
LC-FR1 having the amino acid sequence of SEQ ID NO:288
LC-FR2 having the amino acid sequence of SEQ ID NO:298
LC-FR3 having the amino acid sequence of SEQ ID NO:284
LC-FR4 having the amino acid sequence of SEQ ID NO:47.

8. The antigen-binding molecule according to claim 1, wherein the antigen-binding molecule comprises a heavy chain comprising the amino acid sequence of SEQ ID NO:331.

9. The antigen-binding molecule according to claim 1, wherein the antigen-binding molecule comprises a light chain comprising the amino acid sequence of SEQ ID NO:317.

10. A composition comprising an antigen-binding molecule which is capable of binding to VISTA and inhibiting VISTA-mediated signalling, independently of Fc-mediated function.

11. The composition according to claim 10, wherein the composition comprises:
(i) 2 mM to 200 mM histidine, 2% to 20% (w/v) sucrose, 0.001% to 0.1% (w/v) polysorbate-80, and has a pH 4.0 to 7.0; or
(ii) 2 mM to 200 mM histidine, 2% to 20% (w/v) sucrose, 0.001% to 0.1% (w/v) polysorbate-20, and has a pH 4.0 to 7.0; or
(iii) 2 mM to 200 mM histidine, 1 mM to 250 mM sodium chloride, and has a pH 4.0 to 7.0, optionally comprising 0.001% to 0.1% (w/v) polysorbate-20 or polysorbate-80; or
(iv) 2 mM to 200 mM histidine, 0.001% to 0.1% (w/v) polysorbate-20 or polysorbate-80, and has a pH 4.0 to 7.0; or
(v) 2 mM to 200 mM acetate, 2% to 20% (w/v) sucrose, 0.001% to 0.1% (w/v) polysorbate-80, and has a pH 4.0 to 7.0; or
(vi) 2 mM to 200 mM acetate, 2% to 20% (w/v) sucrose, 0.001% to 0.1% (w/v) polysorbate-20, and has a pH 4.0 to 7.0; or
(vii) 2 mM to 200 mM succinate, 2% to 20% (w/v) sucrose, 0.001% to 0.1% (w/v) polysorbate-80, and has a pH 4.0 to 7.0; or
(viii) 2 mM to 200 mM succinate, 2% to 20% (w/v) sucrose, 0.001% to 0.1% (w/v) polysorbate-20, and has a pH 4.0 to 7.0; or
(ix) 20 mM histidine, 8% (w/v) sucrose; 0.02% (w/v) polysorbate-80, and has a pH 5.5; or
(x) 20 mM histidine, 8% (w/v) sucrose; 0.02% (w/v) polysorbate-80, and has a pH 5.8; or
(xi) 20 mM histidine, 4% (w/v) sucrose; 0.02% (w/v) polysorbate-80, and has a pH 5.8; or
(xii) 20 mM histidine, 2% (w/v) sucrose; 0.02% (w/v) polysorbate-80, and has a pH 5.8; or
(xiii) 20 mM histidine, 8% (w/v) sucrose; 0.02% (w/v) polysorbate-80, and has a pH 6.3; or
(xiv) 20 mM histidine, 8% (w/v) sucrose; 0.02% (w/v) polysorbate-20, and has a pH 5.8; or
(xv) 20 mM acetate, 8% (w/v) sucrose; 0.02% (w/v) polysorbate-80, and has a pH 5.5; or
(xvi) 20 mM succinate, 8% (w/v) sucrose; 0.02% (w/v) polysorbate-80, and has a pH 5.5; or
(xvii) 20 mM histidine, 0.02% (w/v) polysorbate-80, and has a pH 5.8; or
(xviii) 20 mM histidine, 150 mM sodium chloride, and has a pH 5.8.

12. (canceled)

13. The composition according to claim 10, wherein the composition comprises 20 mM histidine, 8% (w/v) sucrose; 0.02% (w/v) polysorbate-80, and has a pH 5.5.

14. The composition according to claim 10, wherein the composition comprises about 50 mg/mL of the antigen-binding molecule.

15.-17. (canceled)

18. A method of treating or preventing a cancer in a subject, the method comprising administering a therapeutically- or prophylactically-effective amount of: (i) an antigen-binding molecule which is capable of binding to VISTA and inhibiting VISTA-mediated signalling, independently of Fc-mediated function, or (ii) a composition comprising an antigen-binding molecule which is capable of binding to VISTA and inhibiting VISTA-mediated signalling, independently of Fc-mediated function.

19. The method according to claim 18, wherein the cancer is characterised by the presence of cells expressing VISTA and/or by signalling mediated by a complex comprising VISTA.

20. The method according to claim 18, wherein the cancer is selected from: a hematological cancer, leukemia (e.g. T cell leukemia), acute myeloid leukemia, lymphoma, B cell lymphoma, T cell lymphoma, multiple myeloma, mesothelioma, a solid tumour, lung cancer, non-small cell lung carcinoma (NSCLC), gastric cancer, gastric carcinoma, colorectal cancer, colorectal carcinoma, colorectal adenocarcinoma, uterine cancer, uterine corpus endometrial carcinoma, breast cancer, triple negative breast cancer (TBNC), triple negative breast invasive carcinoma, invasive ductal carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, bladder cancer, prostate cancer and/or prostate adenocarcinoma.

21. The method according to claim 18, wherein the cancer is triple negative breast cancer (TBNC), non-small cell lung carcinoma (NSCLC) and/or a solid tumour.

22. The method according to claim 18, wherein the method comprises a step of detecting the presence of cells expressing VISTA and/or by signalling mediated by a complex comprising VISTA.

23. The method according to claim 22, wherein the subject is selected for treatment with the antigen-binding molecule or composition when the presence of cells expressing VISTA and/or signalling mediated by a complex comprising VISTA is detected.

24. The method according to claim 18, wherein:
(i) the antigen-binding molecule is administered weekly, every two weeks, or every three weeks; or
(ii) the antigen-binding molecule is administered one, two, or three times within an administration cycle of 21 days, optionally wherein the treatment comprises up to 35 administration cycles; or
(iii) the antigen-binding molecule is administered on days 1, 8 and/or 15 within an administration cycle of 21 days, optionally wherein the treatment comprises up to 35 administration cycles.

25.-26. (canceled)

27. The method according to claim 18, wherein the treatment comprises:
(i) administering 3.5 mg to 2200 mg of antigen-binding molecule per administration; or
(ii) administering at least one of: 3.5 mg, 7 mg, 10.5 mg, 17.5 mg, 20 mg, 21 mg, 40 mg, 60 mg, 72 mg, 120 mg, 180 mg, 240 mg, 360 mg, 400 mg, 800 mg, 1200 mg, 1600 mg, 1900 mg or 2200 mg of antigen-binding molecule per administration; or
(iii) administering up to 10.5 mg, up to 21 mg, up to 31.5 mg, up to 52.5 mg, up to 60 mg, up to 63 mg, up to 120 mg, up to 180 mg, up to 216 mg, up to 360 mg, up to 540 mg, up to 720 mg, up to 1080 mg, up to 1200 mg, up to 2400 mg, up to 3600 mg, up to 4800 mg, up to 5700 mg, or up to 6600 mg of antigen-binding molecule per administration cycle of 21 days.

28.-29. (canceled)


SEQUENCE LISTING (331 sequences)

SEQ ID NO: 1 - protein, 311 aa, synthetic construct
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 2 - protein, 279 aa, synthetic construct
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 3 - protein, 162 aa, synthetic construct
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAA

SEQ ID NO: 4 - protein, 21 aa, synthetic construct
ALATGACIVGILCLPLILLLV

SEQ ID NO: 5 - protein, 96 aa, synthetic construct
YKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 6 - protein, 136 aa, synthetic construct
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQV

SEQ ID NO: 7 - protein, 431 aa, synthetic construct
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 8 - protein, 430 aa, synthetic construct
MSLVELLLWWNCFSRTGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 9 - protein, 406 aa, synthetic construct
MSLVELLLWWNCFSRTGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 10 - protein, 409 aa, synthetic construct
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 11 - protein, 409 aa, synthetic construct; identical to SEQ ID NO: 10

SEQ ID NO: 12 - protein, 385 aa, synthetic construct
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 13 - protein, 219 aa, synthetic construct
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIG

SEQ ID NO: 14 - protein, 195 aa, synthetic construct
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIG

SEQ ID NO: 15 - protein, 21 aa, synthetic construct
LIAGAIGTGAVIIIFCIALIL

SEQ ID NO: 16 - protein, 169 aa, synthetic construct
GAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 17 - protein, 114 aa, synthetic construct
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVT

SEQ ID NO: 18 - protein, 91 aa, synthetic construct
PSAPHCQIQGSQDIGSDVILLCSSEEGIPRPTYLWEKLDNTLKLPPTATQDQVQGTVTIRNISALSSGLYQCVASNAIGTSTCLLDLQVIS

SEQ ID NO: 19 - protein, 414 aa, synthetic construct
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARGAFGYGNGGGVGGGACGDLASEIREDAVAPGCKASGRGSRVTHLLGYPTQNVSRSLRRKYAPPPCGGPEDVALAPCTAAAACEAGPSPVYVKVKSAEPADCAEGPVQCKNGLLV

SEQ ID NO: 20 - protein, 393 aa, synthetic construct
VRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARGAFGYGNGGGVGGGACGDLASEIREDAVAPGCKASGRGSRVTHLLGYPTQNVSRSLRRKYAPPPCGGPEDVALAPCTAAAACEAGPSPVYVKVKSAEPADCAEGPVQCKNGLLV

SEQ ID NO: 21 - protein, 242 aa, synthetic construct
VRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIG

SEQ ID NO: 22 - protein, 21 aa, synthetic construct
VIIGIVLGSLLALGCLAVGIW

SEQ ID NO: 23 - protein, 130 aa, synthetic construct
GLVCCCCGGSGAGGARGAFGYGNGGGVGGGACGDLASEIREDAVAPGCKASGRGSRVTHLLGYPTQNVSRSLRRKYAPPPCGGPEDVALAPCTAAAACEAGPSPVYVKVKSAEPADCAEGPVQCKNGLLV

SEQ ID NO: 24 - protein, 120 aa, synthetic construct
VRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVT

SEQ ID NO: 25 - protein, 112 aa, synthetic construct
PAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVS

SEQ ID NO: 26 - protein, 14 aa, synthetic construct
SRGEVQTCSERRPI

SEQ ID NO: 27 - protein, 14 aa, synthetic construct
FQDLHLHHGGHQAA

SEQ ID NO: 28 - protein, 14 aa, synthetic construct
CLVVEIRHHHSEHR

SEQ ID NO: 29 - protein, 11 aa, synthetic construct
DKGHDVTFYKT

SEQ ID NO: 30 - protein, 11 aa, synthetic construct
RHHHSEHRVHG

SEQ ID NO: 31 - protein, 102 aa, synthetic construct
DKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHG

SEQ ID NO: 32 - protein, 119 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSS

SEQ ID NO: 33 - protein, 9 aa, synthetic construct
GYSITSDYA

SEQ ID NO: 34 - protein, 7 aa, synthetic construct
ITYSGNI

SEQ ID NO: 35 - protein, 12 aa, synthetic construct
ARSLYYPWYFDV

SEQ ID NO: 36 - protein, 25 aa, synthetic construct
EVKLVESGPGLVKPSQSLSLTCTVT

SEQ ID NO: 37 - protein, 17 aa, synthetic construct
WNWIRQFPGNKLEWMGY

SEQ ID NO: 38 - protein, 38 aa, synthetic construct
SYNPSLRSRISITRDTSKNQFFLQLNSVTPEDTATYSC

SEQ ID NO: 39 - protein, 11 aa, synthetic construct
WGAGTTVTVSS

SEQ ID NO: 40 - protein, 107 aa, synthetic construct
DIVITQTPAILSTSPGEKVTMTCRASSSVGYIHWYQQKPGSSPKPWIYATSNLASGVPARFSGSGSGTSNSLTITRVEAEDAATYYCQQWSSYPPITFGGGTKLEVK

SEQ ID NO: 41 - protein, 5 aa, synthetic construct
SSVGY

SEQ ID NO: 42 - skipped

SEQ ID NO: 43 - protein, 10 aa, synthetic construct
QQWSSYPPIT

SEQ ID NO: 44 - protein, 26 aa, synthetic construct
DIVITQTPAILSTSPGEKVTMTCRAS

SEQ ID NO: 45 - protein, 17 aa, synthetic construct
IHWYQQKPGSSPKPWIY

SEQ ID NO: 46 - protein, 36 aa, synthetic construct
NLASGVPARFSGSGSGTSNSLTITRVEAEDAATYYC

SEQ ID NO: 47 - protein, 10 aa, synthetic construct
FGGGTKLEVK

SEQ ID NO: 48 - protein, 119 aa, synthetic construct
EVMLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSS

SEQ ID NO: 49 - protein, 25 aa, synthetic construct
EVMLVESGPGLVKPSQSLSLTCTVT

SEQ ID NO: 50 - protein, 107 aa, synthetic construct
DIVLTQTTAILSTSPGEKVTMTCRASSSVGYIHWYQQKPGSSPKPWIYATSNLASGVPARFSGSGSGTSNSLTITRVEAEDAATYYCQQWSSYPPITFGGGTKLEVK

SEQ ID NO: 51 - protein, 25 aa, synthetic construct
DIVLTQTTAILSTSPGEKVTMTCRA

SEQ ID NO: 52 - protein, 119 aa, synthetic construct
EVQLLESGGGLVQPGGSLRLSCAASGYTITSDYAMSWVRQAPGKGLEWVSVITYSGNISYADSVKGRFTISRDNSKNTLYLQMNSLRAEDTAVYYCARSLYYPWYFDVWGAGTTVTVSS

SEQ ID NO: 53 - protein, 9 aa, synthetic construct
GYTITSDYA

SEQ ID NO: 54 - protein, 25 aa, synthetic construct
EVQLLESGGGLVQPGGSLRLSCAAS

SEQ ID NO: 55 - protein, 17 aa, synthetic construct
MSWVRQAPGKGLEWVSV

SEQ ID NO: 56 - protein, 38 aa, synthetic construct
SYADSVKGRFTISRDNSKNTLYLQMNSLRAEDTAVYYC

SEQ ID NO: 57 - protein, 107 aa, synthetic construct
EIVITQSPATLSLSPGERATLSCRASSSVGYLAWYQQKPGQAPRPLIYDTSNRATGIPARFSGSGSGTDNTLTISSLEPEDFAVYYCQQWSSYPPITFGGGTKLEVK

SEQ ID NO: 58 - skipped

SEQ ID NO: 59 - protein, 26 aa, synthetic construct
EIVITQSPATLSLSPGERATLSCRAS

SEQ ID NO: 60 - protein, 17 aa, synthetic construct
LAWYQQKPGQAPRPLIY

SEQ ID NO: 61 - protein, 36 aa, synthetic construct
NRATGIPARFSGSGSGTDNTLTISSLEPEDFAVYYC

SEQ ID NO: 62 - protein, 119 aa, synthetic construct
QVQLQESGPGLVKPSDTLSLTCTVSGYSITSDYAWSWIRQPPGKGLEWIGYITYSGNISYNPSLKSRVTISVDTSKNQFSLKLSSVTAADTAVYDCARSLYYPWYFDVWGAGTTVTVSS

SEQ ID NO: 63 - protein, 25 aa, synthetic construct
QVQLQESGPGLVKPSDTLSLTCTVS

SEQ ID NO: 64 - protein, 17 aa, synthetic construct
WSWIRQPPGKGLEWIGY

SEQ ID NO: 65 - protein, 38 aa, synthetic construct
SYNPSLKSRVTISVDTSKNQFSLKLSSVTAADTAVYDC

SEQ ID NO: 66 - protein, 107 aa, synthetic construct
DIQMTQSPSSLSASVGDRVTITCRASSSVGYLNWYQQKPGKAPKPLIYAASSLQSGVPSRFSGSGSGTDNTLTISSLQPEDFATYYCQQWSSYPPITFGGGTKLEVK

SEQ ID NO: 67 - skipped

SEQ ID NO: 68 - protein, 25 aa, synthetic construct
DIQMTQSPSSLSASVGDRVTITCRA

SEQ ID NO: 69 - protein, 17 aa, synthetic construct
LNWYQQKPGKAPKPLIY

SEQ ID NO: 70 - protein, 36 aa, synthetic construct
SLQSGVPSRFSGSGSGTDNTLTISSLQPEDFATYYC

SEQ ID NO: 71 - protein, 121 aa, synthetic construct
EVQLQQSGAELVRPGTSVKTSCKASGYTFTNYWLGWVKERAGHGLEWIGEIFPGGGHTNYKEKFKGKATLTADTSSSTAYMKLSSLTSEDSAVYFCAQIPLYYGHYRSAYWGQGTLVTVSA

SEQ ID NO: 72 - protein, 8 aa, synthetic construct
GYTFTNYW

SEQ ID NO: 73 - protein, 8 aa, synthetic construct
IFPGGGHT

SEQ ID NO: 74 - protein, 14 aa, synthetic construct
AQIPLYYGHYRSAY

SEQ ID NO: 75 - protein, 25 aa, synthetic construct
EVQLQQSGAELVRPGTSVKTSCKAS

SEQ ID NO: 76 - protein, 17 aa, synthetic construct
LGWVKERAGHGLEWIGE

SEQ ID NO: 77 - protein, 38 aa, synthetic construct
NYKEKFKGKATLTADTSSSTAYMKLSSLTSEDSAVYFC

SEQ ID NO: 78 - protein, 11 aa, synthetic construct
WGQGTLVTVSA

SEQ ID NO: 79 - protein, 107 aa, synthetic construct
DIQMMQSPASLSASVGETVAITCGASENIYGALNWYQRKQGKSPQLLIYGATNLADGMSSRFSGSGSGRQYSLKISSLHPDDVATYYCQNVLSTPYTFGGGTKLEIK

SEQ ID NO: 80 - protein, 6 aa, synthetic construct
ENIYGA

SEQ ID NO: 81 - skipped

SEQ ID NO: 82 - protein, 9 aa, synthetic construct
QNVLSTPYT

SEQ ID NO: 83 - protein, 26 aa, synthetic construct
DIQMMQSPASLSASVGETVAITCGAS

SEQ ID NO: 84 - protein, 17 aa, synthetic construct
LNWYQRKQGKSPQLLIY

SEQ ID NO: 85 - protein, 36 aa, synthetic construct
NLADGMSSRFSGSGSGRQYSLKISSLHPDDVATYYC

SEQ ID NO: 86 - protein, 10 aa, synthetic construct
FGGGTKLEIK

SEQ ID NO: 87 - protein, 123 aa, synthetic construct
QVTLKECGPGILQPSQTLSLTCSFSGFSLSTSGMGVSWIRQPSGKGLEWLAHIYWDDDKRYNPSLKSRLTISKDSSSNQVFLKITSVDTADTATYYCARRLDGYNDPYYFDYWGQGTTLTVSS

SEQ ID NO: 88 - protein, 10 aa, synthetic construct
GFSLSTSGMG

SEQ ID NO: 89 - protein, 7 aa, synthetic construct
IYWDDDK

SEQ ID NO: 90 - protein, 15 aa, synthetic construct
ARRLDGYNDPYYFDY

SEQ ID NO: 91 - protein, 25 aa, synthetic construct
QVTLKECGPGILQPSQTLSLTCSFS

SEQ ID NO: 92 - protein, 17 aa, synthetic construct
VSWIRQPSGKGLEWLAH

SEQ ID NO: 93 - protein, 38 aa, synthetic construct
RYNPSLKSRLTISKDSSSNQVFLKITSVDTADTATYYC

SEQ ID NO: 94 - protein, 11 aa, synthetic construct
WGQGTTLTVSS

SEQ ID NO: 95 - protein, 107 aa, synthetic construct
DIVMTQSPSSLSASLGDTVTITCHASQNVNVWLSWYQQKPGNIPKLLIYKASNLHAGVPSRFSGSGSGTGFTLTISSLQPEDIATYYCQQGQSYPLTFGGGTKLEIK

SEQ ID NO: 96 - protein, 6 aa, synthetic construct
QNVNVW

SEQ ID NO: 97 - skipped

SEQ ID NO: 98 - protein, 9 aa, synthetic construct
QQGQSYPLT

SEQ ID NO: 99 - protein, 26 aa, synthetic construct
DIVMTQSPSSLSASLGDTVTITCHAS

SEQ ID NO: 100 - protein, 17 aa, synthetic construct
LSWYQQKPGNIPKLLIY

SEQ ID NO: 101 - protein, 36 aa, synthetic construct
NLHAGVPSRFSGSGSGTGFTLTISSLQPEDIATYYC

SEQ ID NO: 102 - protein, 121 aa, synthetic construct
QVQLQQSGAELVRPGTSVKISCKASGYTFTNYWLGWVKERAGHGLEWIGEIFPGGGHTNYKEKFKGKATLTADTSSSTAYMKLSSLTSEDSAVYFCAQIPLYYGHYRSAYWGQGTLVTVSA

SEQ ID NO: 103 - protein, 25 aa, synthetic construct
QVQLQQSGAELVRPGTSVKISCKAS

SEQ ID NO: 104 - protein, 107 aa, synthetic construct
DIVMTQSPASLSASVGETVAITCGASENIYGALNWYQRKQGKSPQLLIYGATNLADGMSSRFSGSGSGRQYSLKISSLHPDDVATYYCQNVLSTPYTFGGGTKLEIK

SEQ ID NO: 105 - protein, 26 aa, synthetic construct
DIVMTQSPASLSASVGETVAITCGAS

SEQ ID NO: 106 - protein, 121 aa, synthetic construct
EVQLQESGPGLVKPSQSLSLTCTVTGYSITSDYAWNWIRQFPGNRLEWMGFISYSGFTTYSPSLESRISITRDTSKNQFFLQLISVTTEDTATYYCARNHYGGSYWYFDVWGAGTSVTVSS

SEQ ID NO: 107 - protein, 7 aa, synthetic construct
ISYSGFT

SEQ ID NO: 108 - protein, 14 aa, synthetic construct
ARNHYGGSYWYFDV

SEQ ID NO: 109 - protein, 25 aa, synthetic construct
EVQLQESGPGLVKPSQSLSLTCTVT

SEQ ID NO: 110 - protein, 17 aa, synthetic construct
WNWIRQFPGNRLEWMGF

SEQ ID NO: 111 - protein, 38 aa, synthetic construct
TYSPSLESRISITRDTSKNQFFLQLISVTTEDTATYYC

SEQ ID NO: 112 - protein, 11 aa, synthetic construct
WGAGTSVTVSS

SEQ ID NO: 113 - protein, 111 aa, synthetic construct
DIVMTQSPGSLAVSLGQRATISCRASESVEYYGTSLMQWYLQKPGQPPKLLIYAASNVESGVPDRFRGSGSGTDFSLNIHPVEEDDIAMYFCQQSRKVPWTFGGGTKLEIK

SEQ ID NO: 114 - protein, 10 aa, synthetic construct
ESVEYYGTSL

SEQ ID NO: 115 - protein, 9 aa, synthetic construct
QQSRKVPWT

SEQ ID NO: 116 - protein, 26 aa, synthetic construct
DIVMTQSPGSLAVSLGQRATISCRAS

SEQ ID NO: 117 - protein, 17 aa, synthetic construct
MQWYLQKPGQPPKLLIY

SEQ ID NO: 118 - protein, 36 aa, synthetic construct
NVESGVPDRFRGSGSGTDFSLNIHPVEEDDIAMYFC

SEQ ID NO: 119 - protein, 119 aa, synthetic construct
EVKVEESGGGLVQPGGSMKLSCAASGFTFSDAWMDWVHQSPEKGLEWVAEIRSKANNHATYYVESVEGRFTISRDDSKSSVFLQVNSLRPEDTGIYYCTRRDGYYFAYWGQGTLVTVSA

SEQ ID NO: 120 - protein, 8 aa, synthetic construct
GFTFSDAW

SEQ ID NO: 121 - protein, 10 aa, synthetic construct
IRSKANNHAT

SEQ ID NO: 122 - protein, 10 aa, synthetic construct
TRRDGYYFAY

SEQ ID NO: 123 - protein, 25 aa, synthetic construct
EVKVEESGGGLVQPGGSMKLSCAAS

SEQ ID NO: 124 - protein, 17 aa, synthetic construct
MDWVHQSPEKGLEWVAE

SEQ ID NO: 125 - protein, 38 aa, synthetic construct
YYVESVEGRFTISRDDSKSSVFLQVNSLRPEDTGIYYC

SEQ ID NO: 126 - protein, 105 aa, synthetic construct
DIVLTQSPAIMSASLGEEITLTCSASSSVRDMHWYQQKSGTSPKVLIYNTFNLASGVPSRFSGSGSGTFYSLTISSVEAGDAAAYYCHQWSSYPTFGGGTKLEIK

SEQ ID NO: 127 - protein, 6 aa, synthetic construct
SSSVRD

SEQ ID NO: 128 - skipped

SEQ ID NO: 129 - protein, 8 aa, synthetic construct
HQWSSYPT

SEQ ID NO: 130 - protein, 25 aa, synthetic construct
DIVLTQSPAIMSASLGEEITLTCSA

SEQ ID NO: 131 - protein, 17 aa, synthetic construct
MHWYQQKSGTSPKVLIY

SEQ ID NO: 132 - protein, 36 aa, synthetic construct
NLASGVPSRFSGSGSGTFYSLTISSVEAGDAAAYYC

SEQ ID NO: 133 - protein, 123 aa, synthetic construct
QVTLKVSGPGILQPSQTLSLTCSFSGFSLSTSGMGVSWIRQPSGKGLEWLAHIYWDDDKRYNPSLKSRLTISKDSSSNQVFLKITSVDTADTATYYCARRLDGYNDPYYFDYWGQGTTLTVSS

SEQ ID NO: 134 - protein, 25 aa, synthetic construct
QVTLKVSGPGILQPSQTLSLTCSFS

SEQ ID NO: 135 - protein, 11 aa, synthetic construct
WGQGTTLTVSS

SEQ ID NO: 136 - protein, 106 aa, synthetic construct
DVVMTQTPALMSASPGEKVTMTCSASSSVSYMYWYQQKPRSSPKPWIYLTSNLASGVPARFSGSGSGTSYSLTISSMEAEDAATYYCQQWNSNPYTFGGGTKLEIK

SEQ ID NO: 137 - protein, 5 aa, synthetic construct
SSVSY

SEQ ID NO: 138 - skipped

SEQ ID NO: 139 - protein, 9 aa, synthetic construct
QQWNSNPYT

SEQ ID NO: 140 - protein, 26 aa, synthetic construct
DVVMTQTPALMSASPGEKVTMTCSAS

SEQ ID NO: 141 - protein, 17 aa, synthetic construct
MYWYQQKPRSSPKPWIY

SEQ ID NO: 142 - protein, 36 aa, synthetic construct
NLASGVPARFSGSGSGTSYSLTISSMEAEDAATYYC

SEQ ID NO: 143 - protein, 118 aa, synthetic construct
QVQLQQSGAELARPGASVRMSCKASGYTFTSYTMNWVKQRPGQGLEWIGFINPDSDYTTYDQKFKDKATLTADSSSSTAYMQLSSLTYDDSAVYYCTRHSYGNYGDYWGQGTTLTVSS

SEQ ID NO: 144 - protein, 8 aa, synthetic construct
GYTFTSYT

SEQ ID NO: 145 - protein, 8 aa, synthetic construct
INPDSDYT

SEQ ID NO: 146 - protein, 11 aa, synthetic construct
TRHSYGNYGDY

SEQ ID NO: 147 - protein, 25 aa, synthetic construct
QVQLQQSGAELARPGASVRMSCKAS

SEQ ID NO: 148 - protein, 17 aa, synthetic construct
MNWVKQRPGQGLEWIGF

SEQ ID NO: 149 - protein, 38 aa, synthetic construct
TYDQKFKDKATLTADSSSSTAYMQLSSLTYDDSAVYYC

SEQ ID NO: 150 - protein, 107 aa, synthetic construct
DIVLTQSPKFLLVSAGDRVTITCKASQSVTNDVAWYQQKPGQSPKLLIYYASSRYTGVPDRFTGSGFGTDFTFTINTVQAEDLAVYFCQQDYSSPYTFGGGTKLEIK

SEQ ID NO: 151 - protein, 6 aa, synthetic construct
QSVTND

SEQ ID NO: 152 - skipped

SEQ ID NO: 153 - protein, 9 aa, synthetic construct
QQDYSSPYT

SEQ ID NO: 154 - protein, 26 aa, synthetic construct
DIVLTQSPKFLLVSAGDRVTITCKAS

SEQ ID NO: 155 - protein, 17 aa, synthetic construct
VAWYQQKPGQSPKLLIY

SEQ ID NO: 156 - protein, 36 aa, synthetic construct
SRYTGVPDRFTGSGFGTDFTFTINTVQAEDLAVYFC

SEQ ID NO: 157 - protein, 118 aa, synthetic construct
QVQLQQSGADLARPGASVKMSCKASGYTFIDYTVHWVKQRPGQGLEWIGFINPSNDYTSYNQKFKDKASLTADTSSTTAYMQLSSLTSDDSAVYYCARHSYGNYGDYWGQGTTLTVSS

SEQ ID NO: 158 - protein, 8 aa, synthetic construct
GYTFIDYT

SEQ ID NO: 159 - protein, 8 aa, synthetic construct
INPSNDYT

SEQ ID NO: 160 - protein, 11 aa, synthetic construct
ARHSYGNYGDY

SEQ ID NO: 161 - protein, 25 aa, synthetic construct
QVQLQQSGADLARPGASVKMSCKAS

SEQ ID NO: 162 - protein, 17 aa, synthetic construct
VHWVKQRPGQGLEWIGF

SEQ ID NO: 163 - protein, 38 aa, synthetic construct
SYNQKFKDKASLTADTSSTTAYMQLSSLTSDDSAVYYC

SEQ ID NO: 164 - protein, 107 aa, synthetic construct
DIVMTQAPKFLLVSAGDRVTITCKASQSVTNGVAWYQQKPGQSPKLLIYYASNRYTGVPDRFTGSGFGTDFTFTISTVQAEDLAVYFCQQDYSSPYTFGGGTKLEIK

SEQ ID NO: 165 - protein, 6 aa, synthetic construct
QSVTNG

SEQ ID NO: 166 - protein, 26 aa, synthetic construct
DIVMTQAPKFLLVSAGDRVTITCKAS

SEQ ID NO: 167 - protein, 36 aa, synthetic construct
NRYTGVPDRFTGSGFGTDFTFTISTVQAEDLAVYFC

SEQ ID NO: 168 - protein, 119 aa, synthetic construct
QVQLQQSGAELVKPGASVRLSCKASGYTFTSYWMHWVRQRPGLGLEWIGEIDPSDSYTNCNQRFKGKATLTVDKSSSTAYTQLSSLTSEDSAVYYCARWAYGPYAMDYWGQGTSVTVSS

SEQ ID NO: 169 - protein, 8 aa, synthetic construct
GYTFTSYW

SEQ ID NO: 170 - protein, 8 aa, synthetic construct
IDPSDSYT

SEQ ID NO: 171 - protein, 12 aa, synthetic construct
ARWAYGPYAMDY

SEQ ID NO: 172 - protein, 25 aa, synthetic construct
QVQLQQSGAELVKPGASVRLSCKAS

SEQ ID NO: 173 - protein, 17 aa, synthetic construct
MHWVRQRPGLGLEWIGE

SEQ ID NO: 174 - protein, 38 aa, synthetic construct
NCNQRFKGKATLTVDKSSSTAYTQLSSLTSEDSAVYYC

SEQ ID NO: 175 - protein, 11 aa, synthetic construct
WGQGTSVTVSS

SEQ ID NO: 176 - protein, 112 aa, synthetic construct
DIVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 177 - protein, 11 aa, synthetic construct
QSLVHSNGNTY

SEQ ID NO: 178 - skipped

SEQ ID NO: 179 - protein, 9 aa, synthetic construct
SQSTHVPWT

SEQ ID NO: 180 - protein, 26 aa, synthetic construct
DIVMTQTPLSLPVSLGDQASISCRSS

SEQ ID NO: 181 - protein, 17 aa, synthetic construct
LHWYLQKPGQSPKLLIY

SEQ ID NO: 182 - protein, 36 aa, synthetic construct [sequence omitted]

SEQ ID NO: 183 - protein, 122 aa, synthetic construct
QVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCARGGYYYGSSWYFDVWGAGTTVTVSS

SEQ ID NO: 184 - protein, 8 aa, synthetic construct
IYPGGGYT

SEQ ID NO: 185 - protein, 15 aa, synthetic construct
ARGGYYYGSSWYFDV

SEQ ID NO: 186 - protein, 17 aa, synthetic construct
LGWVKQRPGHGLEWIGD

SEQ ID NO: 187 - protein, 38 aa, synthetic construct
NYNEKFKGKATLTADTSSSTAYMQLSSLTSEDSAVYFC

SEQ ID NO: 188 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGGGTKLEIK

SEQ ID NO: 189 - protein, 11 aa, synthetic construct
QSIVHSNGNTY

SEQ ID NO: 190 - protein, 9 aa, synthetic construct
FQGSHVPPT

SEQ ID NO: 191 - protein, 26 aa, synthetic construct
DVLMTQTPLSLPVSLGDQASISCRSS

SEQ ID NO: 192 - protein, 17 aa, synthetic construct
LEWYLQKPGQSPKLLIY

SEQ ID NO: 193 - protein, 36 aa, synthetic construct
NRFSGVPDRFSGSGSGTDFTLKISRVEAEDLGVYYC

SEQ ID NO: 194 - protein, 122 aa, synthetic construct
QVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCVRSGYYYGSSWYFDVWGAGTTVTVSS

SEQ ID NO: 195 - protein, 15 aa, synthetic construct
VRSGYYYGSSWYFDV

SEQ ID NO: 196 - protein, 112 aa, synthetic construct
DVLMTQTPLSLPVSLGDQASISCRSSQSIVHSSGNTYLEWYLQKPDQSPKLLIYKVSNRFSGVPDRFSGSGSGTDFTLKISRVEAEDLGVYYCFQGSHVPPTFGGGTKLEIK

SEQ ID NO: 197 - protein, 11 aa, synthetic construct
QSIVHSSGNTY

SEQ ID NO: 198 - protein, 17 aa, synthetic construct
LEWYLQKPDQSPKLLIY

SEQ ID NO: 199 - protein, 122 aa, synthetic construct
QVQLQQSGAELVRPGTSVKISCKASGYTFTNYWLGWVKQRPGHGLEWIGDIYPGGGYTNYNEKFKGKATLTADISSSTAYMQLSSLTSEDSADYFCVRGGYYYGSSWYFDVWGAGTTVTVSS

SEQ ID NO: 200 - protein, 15 aa, synthetic construct
VRGGYYYGSSWYFDV

SEQ ID NO: 201 - protein, 38 aa, synthetic construct
NYNEKFKGKATLTADISSSTAYMQLSSLTSEDSADYFC

SEQ ID NO: 202 - protein, 112 aa, synthetic construct
DVLMTQTPLSLPVSLGDQASISCRSSQSTVHSIGNTYLEWYLQKPGQSPKLLIYKVSNRFSGVPDRFSGSESGTDFTLKISRVEAEDLGVYYCFQGSHVPPTFGGGTKLEIK

SEQ ID NO: 203 - protein, 11 aa, synthetic construct
QSTVHSIGNTY

SEQ ID NO: 204 - protein, 36 aa, synthetic construct
NRFSGVPDRFSGSESGTDFTLKISRVEAEDLGVYYC

SEQ ID NO: 205 - protein, 330 aa, synthetic construct [sequence omitted]

SEQ ID NO: 206 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 207 - protein, 12 aa, synthetic construct
EPKSCDKTHTCP

SEQ ID NO: 208 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 209 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 107 aa, synthetic construct [sequence omitted]

SEQ ID NO: 212 - protein, 449 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 214 aa, synthetic construct
DIVITQTPAILSTSPGEKVTMTCRASSSVGYIHWYQQKPGSSPKPWIYATSNLASGVPARFSGSGSGTSNSLTITRVEAEDAATYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 449 aa, synthetic construct
EVMLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 214 aa, synthetic construct
DIVLTQTTAILSTSPGEKVTMTCRASSSVGYIHWYQQKPGSSPKPWIYATSNLASGVPARFSGSGSGTSNSLTITRVEAEDAATYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 449 aa, synthetic construct
EVQLLESGGGLVQPGGSLRLSCAASGYTITSDYAMSWVRQAPGKGLEWVSVITYSGNISYADSVKGRFTISRDNSKNTLYLQMNSLRAEDTAVYYCARSLYYPWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 214 aa, synthetic construct
EIVITQSPATLSLSPGERATLSCRASSSVGYLAWYQQKPGQAPRPLIYDTSNRATGIPARFSGSGSGTDNTLTISSLEPEDFAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 449 aa, synthetic construct
QVQLQESGPGLVKPSDTLSLTCTVSGYSITSDYAWSWIRQPPGKGLEWIGYITYSGNISYNPSLKSRVTISVDTSKNQFSLKLSSVTAADTAVYDCARSLYYPWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 214 aa, synthetic construct
DIQMTQSPSSLSASVGDRVTITCRASSSVGYLNWYQQKPGKAPKPLIYAASSLQSGVPSRFSGSGSGTDNTLTISSLQPEDFATYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 451 aa, synthetic construct
EVQLQQSGAELVRPGTSVKTSCKASGYTFTNYWLGWVKERAGHGLEWIGEIFPGGGHTNYKEKFKGKATLTADTSSSTAYMKLSSLTSEDSAVYFCAQIPLYYGHYRSAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 214 aa, synthetic construct
DIQMMQSPASLSASVGETVAITCGASENIYGALNWYQRKQGKSPQLLIYGATNLADGMSSRFSGSGSGRQYSLKISSLHPDDVATYYCQNVLSTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 453 aa, synthetic construct
QVTLKECGPGILQPSQTLSLTCSFSGFSLSTSGMGVSWIRQPSGKGLEWLAHIYWDDDKRYNPSLKSRLTISKDSSSNQVFLKITSVDTADTATYYCARRLDGYNDPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 214 aa, synthetic construct
DIVMTQSPSSLSASLGDTVTITCHASQNVNVWLSWYQQKPGNIPKLLIYKASNLHAGVPSRFSGSGSGTGFTLTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 224 - protein, 451 aa, synthetic construct
QVQLQQSGAELVRPGTSVKISCKASGYTFTNYWLGWVKERAGHGLEWIGEIFPGGGHTNYKEKFKGKATLTADTSSSTAYMKLSSLTSEDSAVYFCAQIPLYYGHYRSAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 214 aa, synthetic construct
DIVMTQSPASLSASVGETVAITCGASENIYGALNWYQRKQGKSPQLLIYGATNLADGMSSRFSGSGSGRQYSLKISSLHPDDVATYYCQNVLSTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - protein, 451 aa, synthetic construct
EVQLQESGPGLVKPSQSLSLTCTVTGYSITSDYAWNWIRQFPGNRLEWMGFISYSGFTTYSPSLESRISITRDTSKNQFFLQLISVTTEDTATYYCARNHYGGSYWYFDVWGAGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 218 aa, synthetic construct
DIVMTQSPGSLAVSLGQRATISCRASESVEYYGTSLMQWYLQKPGQPPKLLIYAASNVESGVPDRFRGSGSGTDFSLNIHPVEEDDIAMYFCQQSRKVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 449 aa, synthetic construct
EVKVEESGGGLVQPGGSMKLSCAASGFTFSDAWMDWVHQSPEKGLEWVAEIRSKANNHATYYVESVEGRFTISRDDSKSSVFLQVNSLRPEDTGIYYCTRRDGYYFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 212 aa, synthetic construct
DIVLTQSPAIMSASLGEEITLTCSASSSVRDMHWYQQKSGTSPKVLIYNTFNLASGVPSRFSGSGSGTFYSLTISSVEAGDAAAYYCHQWSSYPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 453 aa, synthetic construct
QVTLKVSGPGILQPSQTLSLTCSFSGFSLSTSGMGVSWIRQPSGKGLEWLAHIYWDDDKRYNPSLKSRLTISKDSSSNQVFLKITSVDTADTATYYCARRLDGYNDPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 213 aa, synthetic construct
DVVMTQTPALMSASPGEKVTMTCSASSSVSYMYWYQQKPRSSPKPWIYLTSNLASGVPARFSGSGSGTSYSLTISSMEAEDAATYYCQQWNSNPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 448 aa, synthetic construct
QVQLQQSGAELARPGASVRMSCKASGYTFTSYTMNWVKQRPGQGLEWIGFINPDSDYTTYDQKFKDKATLTADSSSSTAYMQLSSLTYDDSAVYYCTRHSYGNYGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 214 aa, synthetic construct
DIVLTQSPKFLLVSAGDRVTITCKASQSVTNDVAWYQQKPGQSPKLLIYYASSRYTGVPDRFTGSGFGTDFTFTINTVQAEDLAVYFCQQDYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 234 - protein, 448 aa, synthetic construct
QVQLQQSGADLARPGASVKMSCKASGYTFIDYTVHWVKQRPGQGLEWIGFINPSNDYTSYNQKFKDKASLTADTSSTTAYMQLSSLTSDDSAVYYCARHSYGNYGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 214 aa, synthetic construct
DIVMTQAPKFLLVSAGDRVTITCKASQSVTNGVAWYQQKPGQSPKLLIYYASNRYTGVPDRFTGSGFGTDFTFTISTVQAEDLAVYFCQQDYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 449 aa, synthetic construct
QVQLQQSGAELVKPGASVRLSCKASGYTFTSYWMHWVRQRPGLGLEWIGEIDPSDSYTNCNQRFKGKATLTVDKSSSTAYTQLSSLTSEDSAVYYCARWAYGPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 219 aa, synthetic construct
DIVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 452 aa, synthetic construct
QVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCARGGYYYGSSWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 219 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 452 aa, synthetic construct
QVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCVRSGYYYGSSWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 219 aa, synthetic construct
DVLMTQTPLSLPVSLGDQASISCRSSQSIVHSSGNTYLEWYLQKPDQSPKLLIYKVSNRFSGVPDRFSGSGSGTDFTLKISRVEAEDLGVYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 452 aa, synthetic construct
QVQLQQSGAELVRPGTSVKISCKASGYTFTNYWLGWVKQRPGHGLEWIGDIYPGGGYTNYNEKFKGKATLTADISSSTAYMQLSSLTSEDSADYFCVRGGYYYGSSWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 219 aa, synthetic construct
DVLMTQTPLSLPVSLGDQASISCRSSQSTVHSIGNTYLEWYLQKPGQSPKLLIYKVSNRFSGVPDRFSGSESGTDFTLKISRVEAEDLGVYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 9 aa, synthetic construct
  VARIANT 3: note = X is S or T
GYXITSDYA

SEQ ID NO: 245 - skipped

SEQ ID NO: 246 - protein, 15 aa, synthetic construct
  VARIANT 1: note = X is V or A
  VARIANT 3: note = X is G or S
XRXGYYYGSSWYFDV

SEQ ID NO: 247 - protein, 11 aa, synthetic construct
  VARIANT 3: note = X is I or T
  VARIANT 7: note = X is N, S or I
QSXVHSXGNTY

SEQ ID NO: 248 - protein, 449 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 249 - protein, 449 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 250 - protein, 214 aa, synthetic construct
DIVITQTPAILSTSPGEKVTMTCRASSSVGYIHWYQQKPGSSPKPWIYATSNLASGVPARFSGSGSGTSNSLTITRVEAEDAATYYCQQWSSYPPITFGGGTKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 251 - protein, 97 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 252 - protein, 16 aa, synthetic construct
EPRGPTIKPCPPCKCP

SEQ ID NO: 253 - protein, 110 aa, synthetic construct
APNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPK

SEQ ID NO: 254 - protein, 110 aa, synthetic construct
APNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPK

SEQ ID NO: 255 - protein, 107 aa, synthetic construct
GSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 256 - protein, 330 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 257 - protein, 217 aa, synthetic construct
APNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 258 - protein, 449 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYQSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 259 - protein, 110 aa, synthetic construct
APNLLGGPSVFIFPPKIKDVLMISLSPIVTCVVVDVSEDDPDVQISWFVNNVEVHTAQTQTHREDYQSTLRVVSALPIQHQDWMSGKEFKCKVNNKDLPAPIERTISKPK

SEQ ID NO: 260 - protein, 217 aa, synthetic construct
APNLLGGPSVFIFPPKIKDVLMISLSPIVTCVVVDVSEDDPDVQISWFVNNVEVHTAQTQTHREDYQSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 261 - protein, 324 aa, synthetic construct
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 262 - protein, 97 aa, synthetic construct
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKI

SEQ ID NO: 263 - protein, 13 aa, synthetic construct
VPRDCGCKPCICT

SEQ ID NO: 264 - protein, 107 aa, synthetic construct
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTK

SEQ ID NO: 265 - protein, 107 aa, synthetic construct
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 266 - protein, 443 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 267 - protein, 449 aa, synthetic construct
QVQLQQSGAELMKPGASVKISCKATGYTFSTHWIEWVKQRPGHGLEWIGEILPGSGSTSYNEKFKGKATFTADTSSNTAYMQLSSLTSEDSAVYYCARWLLYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 268 - protein, 219 aa, synthetic construct
DVLMTQTPLSLPVSLGDQASISCRSSQSIVHSNGNTYLEWYLQKPGQSPKLLIYKLSNRFSGVPDRFSGSGSGTDFTLKISRVEAEDLGVYYCFQGSHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 450 aa, synthetic construct
QVQLVQSGAEVKKPGSSVKVSCKASGGTFSSYAISWVRQAPGQGLEWMGGLLPIFGTANYAQKFQGRVTITADESTSTAYMELSSLRSEDTAVYYCARSSYGWSYEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 214 aa, synthetic construct
DIQMTQSPSSLSASVGDRVTITCRASQSIDTRLNWYQQKPGKAPKLLIYSASSLQSGVPSRFSGSGSGTDFTLTISSLQPEDFATYYCQQSAYNPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 10 aa, synthetic construct
PVDKGHDVTF

SEQ ID NO: 272 - protein, 12 aa, synthetic construct
RRPIRNLTFQDL

SEQ ID NO: 273 - protein, 14 aa, synthetic construct
TWYRSSRGEVQTCS

SEQ ID NO: 274 - protein, 17 aa, synthetic construct
EIRHHHSEHRVHGAMEL

SEQ ID NO: 275 - protein, 32 aa, synthetic construct
FKVATPYSLYVCPEGQNVTLTCRLLGPVDKGH

SEQ ID NO: 276 - protein, 119 aa, synthetic construct
QVQLQESGPGLVKPSDTLSLTCTVSGYSITSDYAWNWIRQTPGKGLEWIGYITYSGYISYNPSLRSRVTISRDTSKNQFSLKLSSVTAADTAVYSCARALYYPWYFDVWGTGTTVTVSS

SEQ ID NO: 277 - protein, 7 aa, synthetic construct
ITYSGYI

SEQ ID NO: 278 - protein, 12 aa, synthetic construct
ARALYYPWYFDV

SEQ ID NO: 279 - protein, 17 aa, synthetic construct
WNWIRQTPGKGLEWIGY

SEQ ID NO: 280 - protein, 38 aa, synthetic construct
SYNPSLRSRVTISRDTSKNQFSLKLSSVTAADTAVYSC

SEQ ID NO: 281 - protein, 11 aa, synthetic construct
WGTGTTVTVSS

SEQ ID NO: 282 - protein, 107 aa, synthetic construct
EIVITQSPATLSLSPGERATLSCRASSSVGYIHWYQQKPGQAPRPLIYATSNRATGIPARFSGSGSGTDNTLTISSLEPEDSAVYYCQQWSSYPPITFGGGTKLEVK

SEQ ID NO: 283 - protein, 17 aa, synthetic construct
IHWYQQKPGQAPRPLIY

SEQ ID NO: 284 - protein, 36 aa, synthetic construct
NRATGIPARFSGSGSGTDNTLTISSLEPEDSAVYYC

SEQ ID NO: 285 - protein, 119 aa, synthetic construct
QVQLQESGPGLVKPSDTLSLTCTVSGYSITSDYAWNWIRQTPGKGLEWIGYITYSGYVSYNPSLRSRVTISRDTSKNQFSLKLSSVTAADTAVYSCARALYYPWYFDVWGTGTTVTVSS

SEQ ID NO: 286 - protein, 7 aa, synthetic construct
ITYSGYV

SEQ ID NO: 287 - protein, 107 aa, synthetic construct
EIVLTQSPATLSLSPGERATLSCRASSSVGYIHWYQQKPGQAPRPLIYATSNRATGIPARFSGSGSGTDNTLTISSLEPEDSAVYYCQQWSSYPPITFGGGTKLEVK

SEQ ID NO: 288 - protein, 26 aa, synthetic construct [sequence omitted]

SEQ ID NO: 289 - protein, 119 aa, synthetic construct
QVQLQESGPGLVKPSDTLSLTCTVSGYSITSDYTWNWIRQTPGKGLEWIGHITYSGSVSYNPSLRSRVTISRDTSKNQFSLKLSSVTAADTATYSCARALYYPWYFDVWGTGTTVTVSS

SEQ ID NO: 290 - protein, 9 aa, synthetic construct
GYSITSDYT

SEQ ID NO: 291 - protein, 7 aa, synthetic construct
ITYSGSV

SEQ ID NO: 292 - protein, 17 aa, synthetic construct
WNWIRQTPGKGLEWIGH

SEQ ID NO: 293 - protein, 38 aa, synthetic construct
SYNPSLRSRVTISRDTSKNQFSLKLSSVTAADTATYSC

SEQ ID NO: 294 - protein, 107 aa, synthetic construct
EIVLTQSPATLSLSPGERATLSCRASSSVGYIHWYQQKPGQAPRPLIYTTSYRATGIPARFSGSGSGTDNTLTISSLEPEDSAVYYCQQWSSYPPITFGGGTKLEVK

SEQ ID NO: 295 - skipped

SEQ ID NO: 296 - protein, 36 aa, synthetic construct
YRATGIPARFSGSGSGTDNTLTISSLEPEDSAVYYC

SEQ ID NO: 297 - protein, 107 aa, synthetic construct
EIVLTQSPATLSLSPGERATLSCRASSSVGYIHWYQQKPGQAPRPIIYTTSNRATGIPARFSGSGSGTDNTLTISSLEPEDSAVYYCQQWSSYPPITFGGGTKLEVK

SEQ ID NO: 298 - protein, 17 aa, synthetic construct
IHWYQQKPGQAPRPIIY

SEQ ID NO: 299 - protein, 107 aa, synthetic construct
EIVLTQSPATLSLSPGERATLSCRASSSVGYIHWYQQKPGQAPRPLIYATYNRATGIPARFSGSGSGTDNTLTISSLEPEDSAVYYCQQWSSYPPITFGGGTKLEVK

SEQ ID NO: 300 - skipped

SEQ ID NO: 301 - protein, 107 aa, synthetic construct
EIVLTQSPATLSLSPGERATLSCRASSSVGYIHWYQQKPGQAPRPLIYATSYRATGIPARFSGSGSGTDNTLTISSLEPEDSAVYYCQQWSSYPPITFGGGTKLEVK

SEQ ID NO: 302 - protein, 107 aa, synthetic construct
EIVLTQSPATLSLSPGERATLSCRASSSVGYIHWYQQKPGQAPRPLIYATYYRATGIPARFSGSGSGTDNTLTISSLEPEDSAVYYCQQWSSYPPITFGGGTKLEVK

SEQ ID NO: 303 - protein, 107 aa, synthetic construct
EIVLTQSPATLSLSPGERATLSCRASSSVGYIHWYQQKPGQAPRPLIYATYYRTTGIPARFSGSGSGTDNTLTISSLEPEDSAVYYCQQWSSYPPITFGGGTKLEVK

SEQ ID NO: 304 - protein, 36 aa, synthetic construct
YRTTGIPARFSGSGSGTDNTLTISSLEPEDSAVYYC

SEQ ID NO: 305 - protein, 9 aa, synthetic construct
  VARIANT 3: note = X is S or T
  VARIANT 9: note = X is T or A
GYXITSDYX

SEQ ID NO: 306 - protein, 7 aa, synthetic construct
  VARIANT 6: note = X is S, N or Y
  VARIANT 7: note = X is V or I
ITYSGXX

SEQ ID NO: 307 - protein, 12 aa, synthetic construct
  VARIANT 3: note = X is A or S
ARXLYYPWYFDV

SEQ ID NO: 308-309 - skipped (2 entries)

SEQ ID NO: 310 - protein, 109 aa, synthetic construct
  VARIANT 46: note = X is L or I
  VARIANT 49: note = X is T or A
  VARIANT 51: note = X is S or Y
  VARIANT 52: note = X is N or Y
EIVLTQSPATLSLSPGERATLSCRASSSVGYIHWYQQKPGQAPRPXIYXTXXYNRATGIPARFSGSGSGTDNTLTISSLEPEDSAVYYCQQWSSYPPITFGGGTKLEVK

SEQ ID NO: 311 - protein, 449 aa, synthetic construct
QVQLQESGPGLVKPSDTLSLTCTVSGYSITSDYAWNWIRQTPGKGLEWIGYITYSGYISYNPSLRSRVTISRDTSKNQFSLKLSSVTAADTAVYSCARALYYPWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 312 - protein, 214 aa, synthetic construct
EIVITQSPATLSLSPGERATLSCRASSSVGYIHWYQQKPGQAPRPLIYATSNRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 313 - protein, 449 aa, synthetic construct
QVQLQESGPGLVKPSDTLSLTCTVSGYSITSDYAWNWIRQTPGKGLEWIGYITYSGYVSYNPSLRSRVTISRDTSKNQFSLKLSSVTAADTAVYSCARALYYPWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 214 aa, synthetic construct
EIVLTQSPATLSLSPGERATLSCRASSSVGYIHWYQQKPGQAPRPLIYATSNRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 315 - protein, 449 aa, synthetic construct
QVQLQESGPGLVKPSDTLSLTCTVSGYSITSDYTWNWIRQTPGKGLEWIGHITYSGSVSYNPSLRSRVTISRDTSKNQFSLKLSSVTAADTATYSCARALYYPWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 214 aa, synthetic construct
EIVLTQSPATLSLSPGERATLSCRASSSVGYIHWYQQKPGQAPRPLIYTTSYRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 317 - protein, 214 aa, synthetic construct
EIVLTQSPATLSLSPGERATLSCRASSSVGYIHWYQQKPGQAPRPIIYTTSNRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 318 - protein, 214 aa, synthetic construct
EIVLTQSPATLSLSPGERATLSCRASSSVGYIHWYQQKPGQAPRPLIYATYNRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 319 - protein, 214 aa, synthetic construct
EIVLTQSPATLSLSPGERATLSCRASSSVGYIHWYQQKPGQAPRPLIYATSYRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 320 - protein, 214 aa, synthetic construct
EIVLTQSPATLSLSPGERATLSCRASSSVGYIHWYQQKPGQAPRPLIYATYYRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 321 - protein, 214 aa, synthetic construct
EIVLTQSPATLSLSPGERATLSCRASSSVGYIHWYQQKPGQAPRPLIYATYYRTTGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 322 - protein, 6 aa, synthetic construct
SRGEVQ

SEQ ID NO: 323 - protein, 412 aa, synthetic construct
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 324 - protein, 428 aa, synthetic construct
MAVGASGLEGDKMAGAMPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 325 - protein, 395 aa, synthetic construct
LQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 326 - protein, 303 aa, synthetic construct
LQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQC

SEQ ID NO: 327 - protein, 21 aa, synthetic construct
LLAILILALVATIFFVCTVVL

SEQ ID NO: 328 - protein, 71 aa, synthetic construct
AVRLSRKGHMYPVRNYSPTEMVCISSLLPDGGEGPSATANGGLSKAKSPGLTPEPREDREGDDLTLHSFLP

SEQ ID NO: 329 - protein, 140 aa, synthetic construct
QTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEA

SEQ ID NO: 330 - protein, 446 aa, synthetic construct
EVKLVESGPGLVKPSQSLSLTCTVTGYSITSDYAWNWIRQFPGNKLEWMGYITYSGNISYNPSLRSRISITRDTSKNQFFLQLNSVTPEDTATYSCARSLYYPWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 331 - protein, 446 aa, synthetic construct
QVQLQESGPGLVKPSDTLSLTCTVSGYSITSDYTWNWIRQTPGKGLEWIGHITYSGSVSYNPSLRSRVTISRDTSKNQFSLKLSSVTAADTATYSCARALYYPWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK